(19) United States
(12) Patent Application Publication
(10) Pub. No.: US 2005/0208095 A1
(43) Pub. Date: Sep. 22, 2005

(54) Polymer compositions and methods for their use

(75) Inventors: William L. Hunter, Vancouver (CA); Philip M. Toleikis, Vancouver (CA); David M. Gravett, Vancouver (CA); Arpita Maiti, Vancouver (CA); Richard T. Liggins, Coquitlam (CA); Aniko Takacs-Cox, North Vancouver (CA); Rui Avelar, Vancouver (CA); Troy A. E. Loss, North Vancouver (CA)

Correspondence Address: SEED INTELLECTUAL PROPERTY LAW GROUP PLLC, 701 FIFTH AVENYUE, SUITE 6300, SEATTLE, WA, 98104-7092, US

(73) Assignee: Angiotech International AG, Zug (CH)

(21) Appl. No.: 10/996,354

(22) Filed: Nov. 22, 2004

Related U.S. Application Data
(63) Continuation-in-part of application No. 10/986,231, filed on Nov. 10, 2004.
(60) Provisional application No. 60/586,861, filed on Jul. 9, 2004.
(60) Provisional application No. 60/566,569, filed on Apr. 28, 2004.
(60) Provisional application No. 60/526,541, filed on Dec. 3, 2003.
(60) Provisional application No. 60/525,226, filed on Nov. 24, 2003.
(60) Provisional application No. 60/523,908, filed on Nov. 20, 2003.

(51) Int. Cl. A61F 2/00

(52) U.S. Cl. 424/423

(57) ABSTRACT

Compositions comprising anti-fibrotic agent(s) and/or polymeric compositions can be used in various medical applications including the prevention of surgical adhesions, treatment of inflammatory arthritis, treatment of scars and keloids, the treatment of vascular disease, and the prevention of cartilage loss.

### CROSS-REFERENCES TO RELATED APPLICATIONS

This application is a Continuation-in-part of U.S. application Ser. Nos. 10/986,231, filed Nov. 10, 2004. This application also claims the benefit under 35 U.S.C. 119(e) of U.S. Provisional Application Ser. Nos. 60/586,861, filed Jul. 9, 2004; U.S. Provisional Application entitled “Compositions and Systems for Forming Crosslinked Biomaterials and Associated Methods of Preparation and Use,” (serial number not yet assigned), filed Sep. 17, 2004; U.S. Provisional Application No. 60/566,569, filed Apr. 28, 2004; 60/526,541, filed Dec. 3, 2003; 60/525,226, filed Nov. 24, 2003; and 60/523,908, filed Nov. 20, 2003; which applications are incorporated herein by reference in their entireties.

### BACKGROUND OF THE INVENTION

1. Field of the Invention

This invention relates generally to polymer compositions that include a therapeutic agent (e.g., a fibrosis-inhibiting agent or an anti-infective agent), and to methods of making and using such compositions.

2. Description of the Related Art

Polymeric compositions, particularly those that include synthetic polymers or a combination of synthetic and naturally occurring polymers, have been used in a variety of medical applications, such as the prevention of surgical adhesions, tissue engineering, and as bioadhesive materials. U.S. Pat. No. 5,162,430 describes the use of collagen-synthetic polymer conjugates prepared by covalently binding collagen to synthetic hydrophilic polymers such as various derivatives of polyethylene glycol. In a related patent, U.S. Pat. No. 5,328,955, various activated forms of polyethylene glycol and various linkages are described, which can be used to produce collagen-synthetic polymer conjugates having a range of physical and chemical properties. U.S. Pat. No. 5,324,775 also describes synthetic hydrophilic polyethylene glycol conjugates, but the conjugates involve naturally occurring polymers such as polysaccharides. EP 0 732 109 A1 discloses a crosslinked biomaterial composition that is prepared using a hydrophobic crosslinking agent, or a mixture of hydrophilic and hydrophobic crosslinking agents. U.S. Pat. No. 5,614,587 describes bioadhesives that comprise collagen that is crosslinked using a multifunctionally activated synthetic hydrophilic polymer. U.S. application Ser. No. 08/403,360, filed Mar. 14, 1995, discloses a composition useful in the prevention of surgical adhesions comprising a substrate material and an anti-adhesion binding agent, where the substrate material may comprise collagen and the binding agent may comprise at least one tissue-reactive functional group and at least one substrate-reactive functional group. U.S. application Ser. No. 08/476,825, filed Jun. 7, 1995, discloses bioadhesive compositions comprising collagen crosslinked using a multifunctionally activated synthetic hydrophilic polymer, as well as methods of using such compositions to effect adhesion between a first surface and a second surface, wherein at least one of the first and second surfaces may be a native tissue surface. U.S. Pat. No. 5,874,500 describes a crosslinked polymer composition that comprises one component having multiple nucleophilic groups and another component having multiple electrophilic groups. Covalently bonding of the nucleophilic and electrophilic groups forms a three dimensional matrix that has a variety of medical uses including tissue adhesion, surface coatings for synthetic implants, and drug delivery. More recent developments include the addition of a third component having either nucleophilic or electrophilic groups, as is described in U.S. Pat. No. 6,458,889 to Trollsas et al. U.S. Pat. No. 5,874,500, U.S. Pat. No. 6,051,648 and U.S. Pat. No. 6,312,725 disclose the in situ crosslinking or crosslinked polymers, in particular poly(ethylene glycol) based polymers, to produce a crosslinked composition. West and Hubbell, Biomaterials (1995) 16:1153-1156, disclose the prevention of post-operative adhesions using a photopolymerized polyethylene glycol-co-lactic acid diacrylate hydrogel and a physically crosslinked polyethylene glycol-co-polypropylene glycol hydrogel, POLOXAMER 407 (BASF Corporation, Mount Olive, N.J.). Polymerizable cyanoacrylates have also been described for use as tissue adhesives (Ellis, et al., J. Otolaryngol. 19:68-72 (1990)). Two-part synthetic polymer compositions have been described that, when mixed together, form covalent bonds with one another, as well as with exposed tissue surfaces (PCT WO 97/22371, which corresponds to U.S. application Ser. No. 08/769,806 U.S. Pat. No. 5,874,500).

### BRIEF SUMMARY OF THE INVENTION

Briefly, in one aspect, the present invention provides compositions that contain both an anti-fibrotic agent and either a polymer or a pre-polymer, i.e., a compound that forms a polymer. In one embodiment, these compositions are formed in-situ when precursors thereof are delivered to a site in the body, or a site on an implant. For example, the compositions of the invention include the crosslinked reaction product that forms when two compounds (a multifunctional polynucleophilic compound and a multi-functional polyelectrophilic compound) are delivered to a site in a host (in other words, a patient) in the presence of an anti-fibrotic agent. However, the compositions of the invention also include a mixture of anti-fibrotic agent and a polymer, where the composition can be delivered to a site in a patient's body to achieve beneficial affects, e.g., the beneficial affects described herein.

In some instances, the polymers themselves are useful in various methods, including the prevention of surgical adhesions.

In another aspect, the present invention provides methods for treating and/or preventing surgical adhesions. The surgical adhesions can be the result of, for example, spinal or neurosurgical procedures, of gynecological procedures, of abdominal procedures, of cardiac procedures, of orthopedic procedures, of reconstructive procedures, and cosmetic procedures.

In another aspect, the present invention provides methods for treating or preventing inflammatory arthritis, such as osteoarthritis and rheumatoid arthritis. The method includes delivering to patient in need thereof an anti-fibrotic agent, optionally with a polymer.

In another aspect, the present invention provides for the prevention of cartilage loss as can occur, for example after a joint injury. The method includes delivering to the joint of the patient in need therof an anti-fibrotic agent, optionally with a polymer.

In another aspect, the present invention provides for treating hypertrophic scars and keloids. The method includes delivering to the scar or keloid of the patient in need thereof an anti-fibrotic agent, optionally with a polymer.

In another aspect, the present invention provides a method for the treatment of vascular disease, e.g., stenosis, restenosis or atherosclerosis. The method includes the perivascular delivery of an anti-fibrotic agent.

In one aspect, the present invention provides a method for implanting a medical device comprising: (a) infiltrating a tissue of a host where the medical device is to be, or has been, implanted with i) an anti-fibrotic agent, ii) an anti-infective agent, iii) a polymer; iv) a composition comprising an anti-fibrotic agent and a polymer, v) a composition comprising an anti-infective agent and a polymer, or vi) a composition comprising an anti-fibrotic agent, an anti-infective agent and a polymer, and (b) implanting the medical device into the host.

Optionally, in separate aspects, the invention provides: a method for implanting a medical device comprising: (a) infiltrating a tissue of a host where the medical device is to be, or has been, implanted with an anti-fibrotic agent, and (b) implanting the medical device into the host; a method for implanting a medical device comprising: (a) infiltrating a tissue of a host where the medical device is to be, or has been, implanted with an anti-infective agent, and (b) implanting the medical device into the host; a method for implanting a medical device comprising: (a) infiltrating a tissue of a host where the medical device is to be, or has been, implanted with a polymer; and (b) implanting the medical device into the host; a method for implanting a medical device comprising: (a) infiltrating a tissue of a host where the medical device is to be, or has been, implanted with a composition comprising an anti-fibrotic agent and a polymer, and (b) implanting the medical device into the host; a method for implanting a medical device comprising: (a) infiltrating a tissue of a host where the medical device is to be, or has been, implanted with a composition comprising an anti-infective agent and a polymer, and (b) implanting the medical device into the host; and a method for implanting a medical device comprising: (a) infiltrating a tissue of a host where the medical device is to be, or has been, implanted with a composition comprising an anti-fibrotic agent, an anti-infective agent and a polymer, and (b) implanting the medical device into the host.

These and other aspects of the present invention will become evident upon reference to the following detailed description and attached drawings. In addition, various references are set forth herein which describe in more detail certain procedures and/or compositions, and are therefore incorporated by reference in the entirety.

### BRIEF DESCRIPTION OF THE DRAWINGS

FIG. 1 is a diagram showing how a cell cycle inhibitor acts at one or more of the steps in the biological pathway.

FIG. 2 is a graph showing the results for the screening assay for assessing the effect of mitoxantrone on nitric oxide production by THP-1 macrophages.

FIG. 3 is a graph showing the results for the screening assay for assessing the effect of Bay 11-7082 on TNF-alpha production by THP-1 macrophages.

FIG. 4 is a graph showing the results for the screening assay for assessing the effect of rapamycin concentration for TNFα production by THP-1 macrophages.

FIG. 5 is graph showing the results of a screening assay for assessing the effect of mitoxantrone on proliferation of human fibroblasts.

FIG. 6 is graph showing the results of a screening assay for assessing the effect of rapamycin on proliferation of human fibroblasts.

FIG. 7 is graph showing the results of a screening assay for assessing the effect of paclitaxel on proliferation of human fibroblasts.

FIG. 8 is a picture that shows an uninjured carotid artery from a rat balloon injury model.

FIG. 9 is a picture that shows an injured carotid artery from a rat balloon injury model.

FIG. 10 is a picture that shows a paclitaxel/mesh treated carotid artery in a rat balloon injury model.

FIG. 11A schematically depicts the transcriptional regulation of matrix metalloproteinases.

FIG. 11B is a blot which demonstrates that IL-1 stimulates AP-1 transcriptional activity.

FIG. 11C is a graph which shows that IL-1 induced binding activity decreased in lysates from chondrocytes which were pretreated with paclitaxel.

FIG. 11D is a blot which shows that IL-1 induction increases collagenase and stromelysin in RNA levels in chondrocytes, and that this induction can be inhibited by pretreatment with paclitaxel.

FIGS. 12A-H are blots that show the effect of various anti-microtubule agents in inhibiting collagenase expression.

FIG. 13 is a graph showing the results of a screening assay for assessing the effect of paclitaxel on smooth muscle cell migration.

FIG. 14 is a graph showing the results of a screening assay for assessing the effect of geldanamycin on IL-1β production by THP-1 macrophages.

FIG. 15 is a graph showing the results of a screening assay for assessing the effect of geldanamycin on IL-8 production by THP-1 macrophages.

FIG. 16 is a graph showing the results of a screening assay for assessing the effect of geldanamycin on MCP-1 production by THP-1 macrophages.

FIG. 17 is graph showing the results of a screening assay for assessing the effect of paclitaxel on proliferation of smooth muscle cells.

FIG. 18 is graph showing the results of a screening assay for assessing the effect of paclitaxel for proliferation of the murine RAW 264.7 macrophage cell line.

FIG. 19 is a graph showing the average rank of joint scores of Hartley guinea pig knees with ACL damage treated with paclitaxel. A reduction in score indicates an improvement in cartilage score. The dose response trend is statistically significant (p<0.02).

FIGS. 20A-C are examples of cross sections of Hartley guinea pig knees of control and paclitaxel treated animals. FIG. 20A. Control speciment showing erosion of cartilage to the bone. FIG. 20B. Paclitaxel dose 1 (low dose) showing fraying of cartilage. FIG. 20C. Paclitaxel dose 2 (medium dose) showing minor defects to cartilage.

FIGS. 21A-F are Safranin-O stained histological slides of representative synovial tissues from naive (healthy) knees (FIGS. 21A and 21D) and knees with arthritis induced by administration of albumin in Freund's complete adjuvant (FIGS. 21B and 21C) or carrageenan (FIGS. 21E and 21F). Arthritic knees received either control (FIGS. 21B and 21E) or 20% paclitaxel-loaded microspheres (FIGS. 21C and 21F). The data illustrate decreased proteoglycan red staining in arthritic knees treated with control microspheres and the proteoglycan protection properties of the paclitaxel-loaded formulation.

### DETAILED DESCRIPTION OF THE INVENTION

Definitions

Prior to setting forth the invention, it may be helpful to an understanding thereof to first set forth definitions of certain terms that are used herein.

“Fibrosis,” or “scarring,” or “fibrotic response” refers to the formation of fibrous (scar) tissue in response to injury or medical intervention. Therapeutic agents which inhibit fibrosis or scarring are referred to herein as “fibrosis-inhibiting agents”, “fibrosis-inhibitors”, “anti-scarring agents”, and the like, where these agents inhibit fibrosis through one or more mechanisms including: inhibiting inflammation or the acute inflammatory response, inhibiting migration or proliferation of connective tissue cells (such as fibroblasts, smooth muscle cells, vascular smooth muscle cells), inhibiting angiogenesis, reducing extracellular matrix (ECM) production or promoting ECM breakdown, and/or inhibiting tissue remodeling. When scarring occurs in a confined space (e.g., within a lumen) following surgery or instrumentation (including implantation of a medical device or implant), such that a body passageway (e.g., a blood vessel, the gastrointestinal tract, the respiratory tract, the urinary tract, the female or male reproductive tract, the eustacian tube etc.) is partially or completely obstructed by scar tissue, this is referred to as “stenosis” (narrowing). When scarring subsequently occurs to re-occlude a body passageway after it was initially successfully opened by a surgical intervention (such as placement of a medical device or implant), this is referred to as “restenosis.”

“Host”, “person”, “subject”, “patient” and the like are used synonymously to refer to the living being into which a device or implant of the present invention is implanted.

“Implanted” refers to having completely or partially placed a device or implant within a host. A device is partially implanted when some of the device reaches, or extends to the outside of, a host.

“Inhibit fibrosis”, “reduce fibrosis”, “inhibits scarring” and the like are used synonymously to refer to the action of agents or compositions which result in a statistically significant decrease in the formation of fibrous tissue that can be expected to occur in the absence of the agent or composition.

“Anti-infective agent” refers to an agent or composition which prevents microrganisms from growing and/or slows the growth rate of microorganisms and/or is directly toxic to microorganisms at or near the site of the agent. These processes would be expected to occur at a statistically significant level at or near the site of the agent or composition relative to the effect in the absence of the agent or composition.

“Inhibit infection” refers to the ability of an agent or composition to prevent microorganisms from accumulating and/or proliferating near or at the site of the agent. These processes would be expected to occur at a statistically significant level at or near the site of the agent or composition relative to the effect in the absence of the agent or composition.

“Inhibitor” refers to an agent which prevents a biological process from occurring or slows the rate or degree of occurrence of a biological process. The process may be a general one such as scarring or refer to a specific biological action such as, for example, a molecular process resulting in release of a cytokine.

“Antagonist” refers to an agent which prevents a biological process from occurring or slows the rate or degree of occurrence of a biological process. While the process may be a general one, typically this refers to a drug mechanism where the drug competes with a molecule for an active molecular site or prevents a molecule from interacting with the molecular site. In these situations, the effect is that the molecular process is inhibited.

“Agonist” refers to an agent which stimulates a biological process or rate or degree of occurrence of a biological process. The process may be a general one such as scarring or refer to a specific biological action such as, for example, a molecular process resulting in release of a cytokine.

“Anti-microtubule agents” should be understood to include any protein, peptide, chemical, or other molecule which impairs the function of microtubules, for example, through the prevention or stabilization of polymerization. Compounds that stabilize polymerization of microtubules are referred to herein as “microtubule stabilizing agents.” A wide variety of methods may be utilized to determine the anti-microtubule activity of a particular compound, including for example, assays described by Smith et al. (Cancer Lett 79(2):213-219, 1994) and Mooberry et al., (Cancer Lett. 96(2):261-266, 1995).

“Medical device”, “implant”, “device”, medical device”, “medical implant”, “implant/device” and the like are used synonymously to refer to any object that is designed to be placed partially or wholly within a patient's body for one or more therapeutic or prophylactic purposes such as for restoring physiological function, alleviating symptoms associated with disease, delivering therapeutic agents, and/or repairing, replacing, or augmenting etc. damaged or diseased organs and tissues. While normally composed of biologically compatible synthetic materials (e.g., medical-grade stainless steel, titanium and other metals; polymers such as polyurethane, silicon, PLA, PLGA and other materials) that are exogenous, some medical devices and implants include materials derived from animals (e.g., “xenografts” such as whole animal organs; animal tissues such as heart valves; naturally occurring or chemically-modified molecules such as collagen, hyaluronic acid, proteins, carbohydrates and others), human donors (e.g., “allografts” such as whole organs; tissues such as bone grafts, skin grafts and others), or from the patients themselves (e.g., “autografts” such as saphenous vein grafts, skin grafts, tendon/ligamentmuscle transplants). Representative examples of medical devices that are of particular utility in the present invention include, but are not restricted to, vascular stents, gastrointestinal stents, tracheal/bronchial stents, genital-urinary stents, ENT stents, intra-articular implants, intraocular lenses, implants for hypertrophic scars and keloids, vascular grafts, anastomotic connector devices, implantable sensors, implantable pumps, soft tissue implants (e.g., cosmetic implants and implants for reconstructive surgery), implantable electrical devices, such as implantable neurostimulators and implantable electrical leads, surgical adhesion barriers, glaucoma drainage devices, surgical films and meshes, prosthetic heart valves, tympanostomy tubes, penile implants, endotracheal and tracheostomy tubes, peritoneal dialysis catheters, intracranial pressure monitors, vena cava filters, central venous catheters (CVC's), ventricular assist devices (e.g., LVAD), spinal prostheses, urinary (Foley) catheters, prosthetic bladder sphincters, orthopedic implants, and gastrointestinal drainage tubes.

“Chondroprotection” refers to the prevention of cartilage loss. Cartilage is formed from chondrocytes, and chondroprotection is the protection of the chrondrocytes so that they do not die.

“Release of an agent” refers to a statistically significant presence of the agent, or a subcomponent thereof, which has disassociated from the implant/device and/or remains active on the surface of (or within) the device/implant.

“Biodegradable” refers to materials for which the degradation process is at least partially mediated by, and/or performed in, a biological system. “Degradation” refers to a chain scission process by which a polymer chain is cleaved into oligomers and monomers. Chain scission may occur through various mechanisms, including, for example, by chemical reaction (e.g., hydrolysis) or by a thermal or photolytic process. Polymer degradation may be characterized, for example, using gel permeation chromatography (GPC), which monitors the polymer molecular mass changes during erosion and drug release. Biodegradable also refers to materials may be degraded by an erosion process mediated by, and/or performed in, a biological system. “Erosion” refers to a process in which material is lost from the bulk. In the case of a polymeric system, the material may be a monomer, an oligomer, a part of a polymer backbone, or a part of the polymer bulk. Erosion includes (i) surface erosion, in which erosion affects only the surface and not the inner parts of a matrix; and (ii) bulk erosion, in which the entire system is rapidly hydrated and polymer chains are cleaved throughout the matrix. Depending on the type of polymer, erosion generally occurs by one of three basic mechanisms (see, e.g., Heller, J., CRC Critical Review in Therapeutic Drug Carrier Systems (1984), 1(1), 39-90); Siepmann, J. et al., Adv. Drug Del. Rev. (2001), 48, 229-247): (1) water-soluble polymers that have been insolubilized by covalent cross-links and that solubilize as the cross-links or the backbone undergo a hydrolytic cleavage; (2) polymers that are initially water insoluble are solubilized by hydrolysis, ionization, or pronation of a pendant group; and (3) hydrophobic polymers are converted to small water-soluble molecules by backbone cleavage. Techniques for characterizing erosion include thermal analysis (e.g., DSC), X-ray diffraction, scanning electron microscopy (SEM), electron paramagnetic resonance spectroscopy (EPR), NMR imaging, and recording mass loss during an erosion experiment. For microspheres, photon correlation spectroscopy (PCS) and other particles size measurement techniques may be applied to monitor the size evolution of erodible devices versus time.

As used herein, “analogue” refers to a chemical compound that is structurally similar to a parent compound, but differs slightly in composition (e.g., one atom or functional group is different, added, or removed). The analogue may or may not have different chemical or physical properties than the original compound and may or may not have improved biological and/or chemical activity. For example, the analogue-may be more hydrophilic or it may have altered reactivity as compared to the parent compound. The analogue may mimic the chemical and/or biologically activity of the parent compound (i.e., it may have similar or identical activity), or, in some cases, may have increased or decreased activity. The analogue may be a naturally or non-naturally occurring (e.g., recombinant) variant of the original compound. An example of an analogue is a mutein (i.e., a protein analogue in which at least one amino acid is deleted, added, or substituted with another amino acid). Other types of analogues include isomers (enantiomers, diasteromers, and the like) and other types of chiral variants of a compound, as well as structural isomers. The analogue may be a branched or cyclic variant of a linear compound. For example, a linear compound may have an analogue that is branched or otherwise substituted to impart certain desirable properties (e.g., improve hydrophilicity or bioavailability).

As used herein, “derivative” refers to a chemically or biologically modified version of a chemical compound that is structurally similar to a parent compound and (actually or theoretically) derivable from that parent compound. A “derivative” differs from an “analogue” in that a parent compound may be the starting material to generate a “derivative,” whereas the parent compound may not necessarily be used as the starting material to generate an “analogue.” A derivative may or may not have different chemical or physical properties of the parent compound. For example, the derivative may be more hydrophilic or it may have altered reactivity as compared to the parent compound. Derivatization (i.e., modification) may involve substitution of one or more moieties within the molecule (e.g., a change in functional group). For example, a hydrogen may be substituted with a halogen, such as fluorine or chlorine, or a hydroxyl group (—OH) may be replaced with a carboxylic acid moiety (—COOH). The term “derivative” also includes conjugates, and prodrugs of a parent compound (i.e., chemically modified derivatives which can be converted into the original compound under physiological conditions). For example, the prodrug may be an inactive form of an active agent. Under physiological conditions, the prodrug may be converted into the active form of the compound. Prodrugs may be formed, for example, by replacing one or two hydrogen atoms on nitrogen atoms by an acyl group (acyl prodrugs) or a carbamate group (carbamate prodrugs). More detailed information relating to prodrugs is found, for example, in Fleisher et al., Advanced Drug Delivery Reviews 19 (1996) 115; Design of Prodrugs, H. Bundgaard (ed.), Elsevier, 1985; or H. Bundgaard, Drugs of the Future 16 (1991) 443. The term “derivative” is also used to describe all solvates, for example hydrates or adducts (e.g., adducts with alcohols), active metabolites, and salts of the parent compound. The type of salt that may be prepared depends on the nature of the moieties within the compound. For example, acidic groups, for example carboxylic acid groups, can form, for example, alkali metal salts or alkaline earth metal salts (e.g., sodium salts, potassium salts, magnesium salts and calcium salts, and also salts with physiologically tolerable quaternary ammonium ions and acid addition salts with ammonia and physiologically tolerable organic amines such as, for example, triethylamine, ethanolamine or tris-(2-hydroxyethyl)amine). Basic groups can form acid addition salts, for example with inorganic acids such as hydrochloric acid, sulfuric acid or phosphoric acid, or with organic carboxylic acids and sulfonic acids such as acetic acid, citric acid, benzoic acid, maleic acid, fumaric acid, tartaric acid, methanesulfonic acid or p-toluenesulfonic acid. Compounds which simultaneously contain a basic group and an acidic group, for example a carboxyl group in addition to basic nitrogen atoms, can be present as zwifterions. Salts can be obtained by customary methods known to those skilled in the art, for example by combining a compound with an inorganic or organic acid or base in a solvent or diluent, or from other salts by cation exchange or anion exchange.

“Hyaluronic acid” or “HA” as used herein refers to all forms of hyaluronic acid that are described or referenced herein, including those that have been processed or chemically or physically modified, as well as hyaluronic acid that has been crosslinked (for example, covalently, ionically, thermally or physically). HA is a glycosaminoglycan composed of a linear chain of about 2500 repeating disaccharide units. Each disaccharide unit is composed of an N-acetylglucosamine residue linked to a glucuronic acid. Hyaluronic acid is a natural substance that is found in the extracellular matrix of many tissues including synovial joint fluid, the vitreous humor of the eye, cartilage, blood vessels, skin and the umbilical cord. Commercial forms of hyaluronic acid having a molecular weight of approximately 1.2 to 1.5 million Daltons (Da) are extracted from rooster combs and other animal sources. Other sources of HA include HA that is isolated from cell culture/fermentation processes. Lower molecular weight HA formulations are also available from a variety of commercial sources. The molecule can be of variable lengths (i.e., different numbers of repeating disaccharide units and different chain branching patterns) and can be modified at several sites (through the addition or subtraction of different functional groups) without deviating from the scope of the present invention.

The term “inter-react” refers to the formulation of covalent bonds, noncovalent bonds, or both. The term thus includes crosslinking, which involves both intermolecular crosslinks and optionally intramolecular crosslinks as well, arising from the formation of covalent bonds. Covalent bonding between two reactive groups may be direct, in which case an atom in reactive group is directly bound to an atom in the other reactive group, or it may be indirect, through a linking group. Noncovalent bonds include ionic (electrostatic) bonds, hydrogen bonds, or the association of hydrophobic molecular segments, which may be the same or different. A crosslinked matrix may, in addition to covalent bonds, also include such intermolecular and/or intramolecular noncovalent bonds.

When referring to polymers, the terms “hydrophilic” and “hydrophobic” are generally defined in terms of an HLB value, i.e., a hydrophilic lipophilic balance. A high HLB value indicates a hydrophilic compound, while a low HLB value characterizes a hydrophobic compound. HLB values are well known in the art, and generally range from 1 to 18. Preferred multifunctional compound cores are hydrophilic, although as long as the multifunctional compound as a whole contains at least one hydrophilic component, crosslinkable hydrophobic components may also be present.

The term “synthetic” is used to refer to polymers, compounds and other such materials that are “chemically synthesized.” For example, a synthetic material in the present compositions may have a molecular structure that is identical to a naturally occurring material, but the material per se, as incorporated in the compositions of the invention, has been chemically synthesized in the laboratory or industrially. “Synthetic” materials also include semi-synthetic materials, i.e., naturally occurring materials, obtained from a natural source, that have been chemically modified in some way. Generally, however, the synthetic materials herein are purely synthetic, i.e., they are neither semi-synthetic nor have a structure that is identical to that of a naturally occurring material.

The term “effective amount” refers to the amount of composition required in order to obtain the effect desired. For example, a “tissue growth-promoting amount” of a composition refers to the amount needed in order to stimulate tissue growth to a detectable degree. Tissue, in this context, includes connective tissue, bone, cartilage, epidermis and dermis, blood, and other tissues. The actual amount that is determined to be an effective amount will vary depending on factors such as the size, condition, sex and age of the patient and can be more readily determined by the caregiver.

The term “in situ” as used herein means at the site of administration. Thus, compositions of the invention can be injected or otherwise applied to a specific site within a patient's body, e.g., a site in need of augmentation, and allowed to crosslink at the site of injection. Suitable sites will generally be intradermal or subcutaneous regions for augmenting dermal support, at a bone fracture site for bone repair, within sphincter tissue for sphincter augmentation (e.g., for restoration of continence), within a wound or suture, to promote tissue regrowth; and within or adjacent to vessel anastomoses, to promote vessel regrowth.

The term “aqueous medium” includes solutions, suspensions, dispersions, colloids, and the like containing water. The term “aqueous environment” means an environment containing an aqueous medium. Similarly, the term “dry environment” means an environment that does not contain an aqueous medium.

With regard to nomenclature pertinent to molecular structures, the following definitions apply:

The term “alkyl” as used herein refers to a branched or unbranched saturated hydrocarbon group typically although not necessarily containing 1 to about 24 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl and the like. Generally, although again not necessarily, alkyl groups herein contain 1 to about 12 carbon atoms. The term “lower alkyl” intends an alkyl group of one to six carbon atoms, preferably one to four carbon atoms. “Substituted alkyl” refers to alkyl substituted with one or more substituent groups. “Alkylene,” “lower alkylene” and “substituted alkylene” refer to divalent alkyl, lower alkyl, and substituted alkyl groups, respectively.

The term “aryl” as used herein, and unless otherwise specified, refers to an aromatic substituent containing a single aromatic ring (monocyclic) or multiple aromatic rings that are fused together, linked covalently, or linked to a common group such as a methylene or ethylene moiety. The common linking group may also be a carbonyl as in benzophenone, an oxygen atom as in diphenylether, or a nitrogen atom as in diphenylamine. Preferred aryl groups contain one aromatic ring or two fused or linked aromatic rings, e.g., phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl in which at least one carbon atom is replaced with a heteroatom. The terms “arylene” and “substituted arylene” refer to divalent aryl and substituted aryl groups as just defined.

The term “heteroatom-containing” as in a “heteroatom-containing hydrocarbyl group” refers to a molecule or molecular fragment in which one or more carbon atoms is replaced with an atom other than carbon, e.g., nitrogen, oxygen, sulfur, phosphorus or silicon.

“Hydrocarbyl” refers to univalent hydrocarbyl radicals containing 1 to about 30 carbon atoms, preferably 1 to about 24 carbon atoms, most preferably 1 to about 12 carbon atoms, including branched or unbranched, saturated or unsaturated species, such as alkyl groups, alkenyl groups, aryl groups, and the like. The term “lower hydrocarbyl” intends a hydrocarbyl group of one to six carbon atoms, preferably one to four carbon atoms. The term “hydrocarbylene” intends a divalent hydrocarbyl moiety containing 1 to about 30 carbon atoms, preferably 1 to about 24 carbon atoms, most preferably 1 to about 12 carbon atoms, including branched or unbranched, saturated or unsaturated species, or the like. The term “lower hydrocarbylene” intends a hydrocarbylene group of one to six carbon atoms, preferably one to four carbon atoms. “Substituted hydrocarbyl” refers to hydrocarbyl substituted with one or more substituent groups, and the terms “heteroatom-containing hydrocarbyl” and “heterohydrocarbyl” refer to hydrocarbyl in which at least one carbon atom is replaced with a heteroatom. Similarly, “substituted hydrocarbylene” refers to hydrocarbylene substituted with one or more substituent groups, and the terms 37 heteroatom-containing hydrocarbylene” and “heterohydrocarbylene” refer to hydrocarbylene in which at least one carbon atom is replaced with a heteroatom. If not otherwise indicated, “hydrocarbyl” indicates both unsubstituted and substituted hydrocarbyls, “heteroatom-containing hydrocarbyl” indicates both unsubstituted and substituted heteroatom-containing hydrocarbyls and so forth.

By “substituted” as in “substituted hydrocarbyl,” “substituted alkyl,” and the like, as alluded to in some of the aforementioned definitions, is meant that in the hydrocarbyl, alkyl, or other moiety, at least one hydrogen atom bound to a carbon atom is replaced with one or more substituents that are functional groups such as alkoxy, hydroxy, halo, nitro, and the like. Unless otherwise indicated, it is to be understood that specified molecular segments can be substituted with one or more substituents that do not compromise a compound's utility. For example, “succinimidyl” is intended to include unsubstituted succinimidyl as well as sulfosuccinimidyl and other succinimidyl groups substituted on a ring carbon atom, e.g., with alkoxy substituents, polyether substituents, or the like.

Any concentration ranges, percentage range, or ratio range recited herein are to be understood to include concentrations, percentages or ratios of any integer within that range and fractions thereof, such as one tenth and one hundredth of an integer, unless otherwise indicated. Also, any number range recited herein relating to any physical feature, such as polymer subunits, size or thickness, are to be understood to include any integer within the recited range, unless otherwise indicated. As used herein, the term “about” refers to ±15% of any indicated structure, value, or range.

“A” and “an” refer to one or more of the indicated items. For example, “a” polymer refers to both one polymer or a mixture comprising two or more polymers; “a multifunctional compound” refers not only to a single multifunctional compound but also to a combination of two or more of the same or different multifunctional compounds; “a reactive group” refers to a combination of reactive groups as well as to a single reactive group, and the like.

As discussed above, the present invention provides polymeric compositions which greatly increase the ability to inhibit the formation of reactive scar tissue on, or around, the surface of a device or implant or at a treatment site. Numerous polymeric compositions and therapeutic agents are described herein.

The present invention provides for the combination of compositions (e.g., polymers) which include one or more therapeutic agents, described below. Also described in more detail below are methods for making and methods for utilizing such compositions.

A. Therapeutic Agents

In one aspect, the present invention discloses pharmaceutical agents which inhibit one or more aspects of the production of excessive fibrous (scar) tissue. Suitable fibrosis-inhibiting or stenosis-inhibiting agents may be readily determined based upon the in vitro and in vivo (animal) models such as those provided in Examples 20-33. Agents which inhibit fibrosis may be identified through in vivo models including inhibition of intimal hyperplasia development in the rat balloon carotid artery model (Examples 25 and 33). The assays set forth in Examples 24 and 32 may be used to determine whether an agent is able to inhibit cell proliferation in fibroblasts and/or smooth muscle cells. In one aspect of the invention, the agent has an IC₅₀ for inhibition of cell proliferation within a range of about 10⁻⁶ to about 10⁻¹⁰ M. The assay set forth in Example 28 may be used to determine whether an agent may inhibit migration of fibroblasts and/or smooth muscle cells. In one aspect of the invention, the agent has an IC₅₀ for inhibition of cell migration within a range of about 10⁻⁶ to about 10⁻⁹M. Assays set forth herein may be used to determine whether an agent is able to inhibit inflammatory processes, including nitric oxide production in macrophages (Example 20), and/or TNF-alpha production by macrophages (Example 21), and/or IL-1 beta production by macrophages (Example 29), and/or IL-8 production by macrophages (Example 30), and/or inhibition of MCP-1 by macrophages (Example 31). In one aspect of the invention, the agent has an IC₅₀ for inhibition of any one of these inflammatory processes within a range of about 10⁻⁶ to about 10⁻¹⁰M. The assay set forth in Example 26 may be used to determine whether an agent is able to inhibit MMP production. In one aspect of the invention, the agent has an IC₅₀ for inhibition of MMP production within a range of about 10⁻⁴ to about 10⁻⁸M. The assay set forth in Example 27 (also known as the CAM assay) may be used to determine whether an agent is able to inhibit angiogenesis. In one aspect of the invention, the agent has an IC₅₀ for inhibition of angiogenesis within a range of about 10⁻⁶ to about 10⁻¹M. Agents which reduce the formation of surgical adhesions may be identified through in vivo models including the rabbit surgical adhesions model (Examples 23, 42 and 43) and the rat caecal sidewall model (Example 22). These pharmacologically active agents (described below) can then be delivered at appropriate dosages into to the tissue either alone, or via carriers (described herein), to treat the clinical problems described herein.

Numerous therapeutic compounds capable of inhibiting fibrosis have been identified that are of utility in the invention including:

1) Angiogenesis Inhibitors

In one embodiment, the pharmacologically active fibrosis-inhibiting compound is an angiogenesis inhibitor (e.g., 2-ME (NSC-659853), PI-88 (D-mannose, O-6-O-phosphono-alpha-D-mannopyranosyl-(1-3)-O-alpha-D-mannopyranosyl-(1-3)-O-alpha-D-mannopyranosyl-(1-3)-O-alpha-D-mannopyranosyl-(1-2)-hydrogen sulfate), thalidomide (1H-isoindole-1,3(2H)-dione, 2-(2,6-dioxo-3-piperidinyl)-), CDC-394, CC-5079, ENMD-0995 (S-3-amino-phthalidoglutarimide), AVE-8062A, vatalanib, SH-268, halofuginone hydrobromide, atiprimod dimaleate (2-azaspivo(4.5)decane-2-propanamine, N,N-diethyl-8,8-dipropyl, dimaleate), ATN-224, CHIR-258, combretastatin A-4 (phenol, 2-methoxy-5-(2-(3,4,5-trimethoxyphenyl)ethenyl)-, (Z)-), GCS-100LE, or an analogue or derivative thereof).

2) 5-Lipoxygenase Inhibitors and Antagonists

In another embodiment, the pharmacologically active fibrosis-inhibiting compound is a 5-lipoxygenase inhibitor or antagonist (e.g., Wy-50295 (2-naphthaleneacetic acid, alpha-methyl-6-(2-quinolinylmethoxy)-, (S)-), ONO-LP-269 (2,11,14-eicosatrienamide, N-(4-hydroxy-2-(1H-tetrazol-5-yl)-8-quinolinyl)-, (E,Z,Z)-), licofelone (1H-pyrrolizine-5-acetic acid, 6-(4-chlorophenyl)-2,3-dihydro-2,2-dimethyl-7-phenyl-), CMI-568 (urea, N-butyl-N-hydroxy-N′-(4-(3-(methylsulfonyl)-2-propoxy-5-(tetrahydro-5-(3,4,5-trimethoxyphenyl)-2-furanyl)phenoxy)butyl)-,trans-), IP-751 ((3R,4R)-(delta 6)-THC-DMH-11-oic acid), PF-5901 (benzenemethanol, alpha-pentyl-3-(2-quinolinylmethoxy)-), LY-293111 (benzoic acid, 2-(3-(3-((5-ethyl-4′-fluoro-2-hydroxy(1,1′-biphenyl)-4-yl)oxy)propoxy)-2-propylphenoxy)-), RG-5901-A (benzenemethanol, alpha-pentyl-3-(2-quinolinylmethoxy)-, hydrochloride), rilopirox (2(1H )-pyridinone, 6-((4-(4-chlorophenoxy)phenoxy)methyl)-1-hydroxy-4-methyl-), L-674636 (acetic acid, ((4-(4-chlorophenyl)-1-(4-(2-quinolinylmethoxy)phenyl)butyl)thio)-AS)), 7-((3-(4-methoxy-tetrahydro-2H-pyran-4-yl)phenyl)methoxy)-4-phenylnaphtho(2,3-c)furan-1 (3H)-one, MK-886 (1H-indole-2-propanoic acid, 1-((4-chlorophenyl)methyl)-3-((1,1-dimethylethyl)thio)-alpha, alpha-dimethyl-5-(1-methylethyl)-), quiflapon (1H-indole-2-propanoic acid, 1-((4-chlorophenyl)methyl)-3-((1,1-dimethylethyl)thio)-alpha, alpha-dimethyl-5-(2-quinolinylmethoxy)-), quiflapon (1H-Indole-2-propanoic acid,1-((4-chlorophenyl)methyl)-3-((1,1-dimethylethyl)thio)-alpha, alpha-dimethyl-5-(2-quinolinylmethoxy)-), docebenone (2,5-cyclohexadiene-1,4-dione, 2-(12-hydroxy-5,10-dodecadiynyl)-3,5,6-trimethyl-), zileuton (urea, N-(1-benzo(b)thien-2-ylethyl)-N-hydroxy-), or an analogue or derivative thereof).

3) Chemokine Receptor Antagonists CCR (1, 3, and 5)

In another embodiment, the pharmacologically active fibrosis-inhibiting compound is a chemokine receptor antagonist which inhibits one or more subtypes of CCR (1, 3, and 5) (e.g., ONO-4128 (1,4,9-triazaspiro(5.5)undecane-2,5-dione, 1-butyl-3-(cyclohexylmethyl)-9-((2,3-dihydro-1,4-benzodioxin-6-yl)methyl-), L-381, CT-112 (L-arginine, L-threonyl-L-threonyl-L-seryl-L-glutaminyl-L-valyl-L-arginyl-L-prolyl-), AS-900004, SCH-C, ZK-811752, PD-172084, UK-427857, SB-380732, vMIP II, SB-265610, DPC-168, TAK-779 (N,N-dimethyl-N-(4-(2-(4-methylphenyl)-6,7-dihydro-5H-benzocyclohepten-8-ylcarboxamido)benyl)tetrahydro-2H-pyran-4-aminium chloride), TAK-220, KRH-1120), GSK766994, SSR-150106, or an analogue or derivative thereof). Other examples of chemokine receptor antagonists include a-lmmunokine-NNS03, BX-471, CCX-282, Sch-350634; Sch-351125; Sch-417690; SCH-C, and analogues and derivatives thereof.

4) Cell Cycle Inhibitors

In another embodiment, the pharmacologically active fibrosis-inhibiting compound is a cell cycle inhibitor. Representative examples of such agents include taxanes (e.g., paclitaxel (discussed in more detail below) and docetaxel) (Schiff et al., Nature 277:665-667, 1979; Long and Fairchild, Cancer Research 54:4355-4361, 1994; Ringel and Horwitz, J. Nat'l Cancer Inst. 83(4):288-291, 1991; Pazdur et al., Cancer Treat. Rev. 19(40):351-386, 1993), etanidazole, nimorazole (B. A. Chabner and D. L. Longo. Cancer Chemotherapy and Biotherapy—Principles and Practice. Lippincott-Raven Publishers, New York, 1996, p.554), perfluorochemicals with hyperbaric oxygen, transfusion, erythropoietin, BW12C, nicotinamide, hydralazine, BSO, WR-2721, ludR, DUdR, etanidazole, WR-2721, BSO, mono-substituted keto-aldehyde compounds (L. G. Egyud. Keto-aldehyde-amine addition products and method of making same. U.S. Pat. No. 4,066,650, Jan. 3, 1978), nitroimidazole (K. C. Agrawal and M. Sakaguchi. Nitroimidazole radiosensitizers for Hypoxic tumor cells and compositions thereof. U.S. Pat. No. 4,462,992, Jul. 31, 1984), 5-substituted-4-nitroimidazoles (Adams et al., Int. J. Radiat. Biol. Relat. Stud. Phys., Chem. Med. 40(2):153-61, 1981), SR-2508 (Brown et al., Int. J. Radiat. Oncol., Biol. Phys. 7(6):695-703, 1981), 2H-isoindolediones (J. A. Myers, 2H-lsoindolediones, the synthesis and use as radiosensitizers. U.S. Pat. No. 4,494,547, Jan. 22, 1985), chiral (((2-bromoethyl)-amino)methyl)-nitro-1H-imidazole-1-ethanol (V. G. Beylin, et al., Process for preparing chiral (((2-bromoethyl)-amino)methyl)-nitro-1H-imidazole-1-ethanol and related compounds. U.S. Pat. No. 5,543,527, Aug. 6, 1996; U.S. Pat. No. 4,797,397; Jan. 10, 1989; U.S. Pat. No. 5,342,959, Aug. 30, 1994), nitroaniline derivatives (W. A. Denny, et al. Nitroaniline derivatives and the use as anti-tumor agents. U.S. Pat. No. 5,571,845, Nov. 5, 1996), DNA-affinic hypoxia selective cytotoxins (M. V. Papadopoulou-Rosenzweig. DNA-affinic hypoxia selective cytotoxins. U.S. Pat. No. 5,602,142, Feb. 11, 1997), halogenated DNA ligand (R. F. Martin. Halogenated DNA ligand radiosensitizers for cancer therapy. U.S. Pat. No. 5,641,764, Jun. 24, 1997), 1,2,4 benzotriazine oxides (W. W. Lee et al. 1,2,4-benzotriazine oxides as radiosensitizers and selective cytotoxic agents. U.S. Pat. No. 5,616,584, Apr. 1, 1997; U.S. Pat. No. 5,624,925, Apr. 29, 1997; Process for Preparing 1,2,4 Benzotriazine oxides. U.S. Pat. No. 5,175,287, Dec. 29, 1992), nitric oxide (J. B. Mitchell et al., Use of Nitric oxide releasing compounds as hypoxic cell radiation sensitizers. U.S. Pat. No. 5,650,442, Jul. 22, 1997), 2-nitroimidazole derivatives (M. J. Suto et al. 2-Nitroimidazole derivatives useful as radiosensitizers for hypoxic tumor cells. U.S. Pat. No. 4,797,397, Jan. 10, 1989; T. Suzuki. 2-Nitroimidazole derivative, production thereof, and radiosensitizer containing the same as active ingredient. U.S. Pat. No. 5,270,330, Dec. 14, 1993; T. Suzuki et al. 2-Nitroimidazole derivative, production thereof, and radiosensitizer containing the same as active ingredient. U.S. Pat. No. 5,270,330, Dec. 14, 1993; T. Suzuki. 2-Nitroimidazole derivative, production thereof and radiosensitizer containing the same as active ingredient; Patent EP 0 513 351 B1, Jan. 24, 1991), fluorine-containing nitroazole derivatives (T. Kagiya. Fluorine-containing nitroazole derivatives and radiosensitizer comprising the same. U.S. Pat. No. 4,927,941, May 22, 1990), copper (M. J. Abrams. Copper Radiosensitizers. U.S. Pat. No. 5,100,885, Mar. 31, 1992), combination modality cancer therapy (D. H. Picker et al. Combination modality cancer therapy. U.S. Pat. No. 4,681,091, Jul. 21, 1987). 5-CldC or (d)H₄U or 5-halo-2′-halo-2′-deoxy-cytidine or -uridine derivatives (S. B. Greer. Method and Materials for sensitizing neoplastic tissue to radiation. U.S. Pat. No. 4,894,364 Jan. 16, 1990); platinum complexes (K. A. Skov. Platinum Complexes with one radiosensitizing ligand. U.S. Pat. No. 4,921,963. May 1, 1990; K. A. Skov. Platinum Complexes with one radiosensitizing ligand. Patent EP 0 287 317 A3), fluorine-containing nitroazole (T. Kagiya, et al. Fluorine-containing nitroazole derivatives and radiosensitizer comprising the same. U.S. Pat. No. 4,927,941. May 22, 1990), benzamide (W. W. Lee. Substituted Benzamide Radiosensitizers. U.S. Pat. No. 5,032,617, Jul. 16, 1991), autobiotics (L. G. Egyud. Autobiotics and the use in eliminating nonself cells in vivo. U.S. Pat. No. 5,147,652. Sep. 15, 1992), benzamide and nicotinamide (W. W. Lee et al. Benzamide and Nictoinamide Radiosensitizers. U.S. Pat. No. 5,215,738, Jun. 1, 1993), acridine-intercalator (M. Papadopoulou-Rosenzweig. Acridine Intercalator based hypoxia selective cytotoxins. U.S. Pat. No. 5,294,715, Mar. 15, 1994), fluorine-containing nitroimidazole (T. Kagiya et al. Fluorine containing nitroimidazole compounds. U.S. Pat. No. 5,304,654, Apr. 19, 1994), hydroxylated texaphyrins (J. L. Sessler et al. Hydroxylated texaphrins. U.S. Pat. No. 5,457,183, Oct. 10, 1995), hydroxylated compound derivative (T. Suzuki et al. Heterocyclic compound derivative, production thereof and radiosensitizer and antiviral agent containing said derivative as active ingredient. Publication Number 011106775 A (Japan), Oct. 22, 1987; T. Suzuki et al. Heterocyclic compound derivative, production thereof and radiosensitizer, antiviral agent and anti cancer agent containing said derivative as active ingredient. Publication Number 01139596 A (Japan), Nov. 25, 1987; S. Sakaguchi et al. Heterocyclic compound derivative, its production and radiosensitizer containing said derivative as active ingredient; Publication Number 63170375 A (Japan), Jan. 7, 1987), fluorine containing 3-nitro-1,2,4-triazole (T. Kagitani et al. Novel fluorine-containing 3-nitro-1,2,4-triazole and radiosensitizer containing same compound. Publication Number 02076861 A (Japan), Mar. 31, 1988), 5-thiotretrazole derivative or its salt (E. Kano et al. Radiosensitizer for Hypoxic cell. Publication Number 61010511 A (Japan), Jun. 26, 1984), Nitrothiazole (T. Kagitani et al. Radiation-sensitizing agent. Publication Number 61167616 A (Japan) Jan. 22, 1985), imidazole derivatives (S. Inayma et al. Imidazole derivative. Publication Number 6203767 A (Japan) Aug. 1, 1985; Publication Number 62030768 A (Japan) Aug. 1, 1985; Publication Number 62030777 A (Japan) Aug. 1, 1985), 4-nitro-1,2,3-triazole (T. Kagitani et al. Radiosensitizer. Publication Number 62039525 A (Japan), Aug. 15, 1985), 3-nitro-1,2,4-triazole (T. Kagitani et al. Radiosensitizer. Publication Number 62138427 A (Japan), Dec. 12, 1985), Carcinostatic action regulator (H. Amagase. Carcinostatic action regulator. Publication Number 63099017 A (Japan), Nov. 21, 1986), 4,5-dinitroimidazole derivative (S. Inayama. 4,5-Dinitroimidazole derivative. Publication Number 63310873 A (Japan) Jun. 9, 1987), nitrotriazole Compound (T. Kagitanil Nitrotriazole Compound. Publication Number 07149737 A (Japan) Jun. 22, 1993), cisplatin, doxorubin, misonidazole, mitomycin, tiripazamine, nitrosourea, mercaptopurine, methotrexate, flurouracil, bleomycin, vincristine, carboplatin, epirubicin, doxorubicin, cyclophosphamide, vindesine, etoposide (I. F. Tannock. Review Article: Treatment of Cancer with Radiation and Drugs. Journal of Clinical Oncology 14(12):3156-3174, 1996), camptothecin (Ewend M. G. et al. Local delivery of chemotherapy and concurrent external beam radiotherapy prolongs survival in metastatic brain tumor models. Cancer Research 56(22):5217-5223, 1996) and paclitaxel (Tishler R. B. et al. Taxol: a novel radiation sensitizer. International Journal of Radiation Oncology and Biological Physics 22(3):613-617, 1992).

A number of the above-mentioned cell cycle inhibitors also have a wide variety of analogues and derivatives, including, but not limited to, cisplatin, cyclophosphamide, misonidazole, tiripazamine, nitrosourea, mercaptopurine, methotrexate, flurouracil, epirubicin, doxorubicin, vindesine and etoposide. Analogues and derivatives include (CPA)₂Pt(DOLYM) and (DACH)Pt(DOLYM) cisplatin (Choi et al., Arch. Pharmacal Res. 22(2):151-156, 1999), Cis-(PtCl₂(4,7-H-5-methyl-7-oxo)1,2,4(triazolo(1,5-a)pyrimidine)₂) (Navarro et al., J. Med. Chem. 41(3):332-338, 1998), (Pt(cis-1,4-DACH)(trans-Cl₂)(CBDCA))·½MeOH cisplatin (Shamsuddin et al., Inorg. Chem. 36(25):5969-5971, 1997), 4-pyridoxate diammine hydroxy platinum (Tokunaga et al., Pharm. Sci. 3(7):353-356, 1997), Pt(II) . . . Pt(II) (Pt₂(NHCHN(C(CH₂)(CH₃)))₄) (Navarro et al., Inorg. Chem. 35(26):7829-7835, 1996), 254-S cisplatin analogue (Koga et al., Neurol. Res. 18(3):244-247, 1996), o-phenylenediamine ligand bearing cisplatin analogues (Koeckerbauer & Bednarski, J. Inorg. Biochem. 62(4):281-298, 1996), trans,cis-(Pt(OAc)₂I₂(en)) (Kratochwil et al., J. Med. Chem. 39(13):2499-2507, 1996), estrogenic 1,2-diarylethylenediamine ligand (with sulfur-containing amino acids and glutathione) bearing cisplatin analogues (Bednarski, J. Inorg. Biochem. 62(1):75, 1996), cis-1,4-diaminocyclohexane cisplatin analogues (Shamsuddin et al., J. Inorg. Biochem. 61(4):291-301, 1996), 5′ orientational isomer of cis-(Pt(NH₃)(4-aminoTEMP-O){d(GpG)}) (Dunham & Lippard, J. Am. Chem. Soc. 117(43):10702-12, 1995), chelating diamine-bearing cisplatin analogues (Koeckerbauer & Bednarski, J. Pharm. Sci. 84(7):819-23, 1995), 1,2-diarylethyleneamine ligand-bearing cisplatin analogues (Ofto et al., J. Cancer Res. Clin. Oncol. 121(1):31-8, 1995), (ethylenediamine)platinum(II) complexes (Pasini et al., J. Chem. Soc., Dalton Trans. 4:579-85, 1995), CI-973 cisplatin analogue (Yang et al., Int. J. Oncol. 5(3):597-602, 1994), cis-diamminedichloroplatinum(II) and its analogues cis-1,1-cyclobutanedicarbosylato(2R)-2-methyl-1,4-butanediam-mineplatinum(II) and cis-diammine(glycolato)platinum (Claycamp & Zimbrick, J. Inorg. Biochem., 26(4):257-67, 1986; Fan et al., Cancer Res. 48(11):3135-9, 1988; Heiger-Bernays et al., Biochemistry 29(36):8461-6, 1990; Kikkawa et al., J. Exp. Clin. Cancer Res. 12(4):233-40, 1993; Murray et al., Biochemistry 31(47):11812-17, 1992; Takahashi et al., Cancer Chemother. Pharmacol. 33(1):31-5, 1993), cis-amine-cyclohexylamine-dichloroplatinum(II) (Yoshida et al., Biochem. Pharmacol. 48(4):793-9, 1994), gem-diphosphonate cisplatin analogues (FR 2683529), (meso-1,2-bis(2,6-dichloro-4-hydroxyplenyl)ethylenediamine) dichloroplatinum(II) (Bednarski et al., J. Med. Chem. 35(23):4479-85, 1992), cisplatin analogues containing a tethered dansyl group (Hartwig et al., J. Am. Chem. Soc. 114(21):8292-3, 1992), platinum(II) polyamines (Siegmann et al., Inorg. Met.—Containing Polym. Mater., (Proc. Am. Chem. Soc. Int. Symp.), 335-61, 1990), cis-(3H)dichloro(ethylenediamine)platinum(II) (Eastman, Anal. Biochem. 197(2):311-15, 1991), trans-diamminedichloroplatinum(II) and cis-(Pt(NH₃)₂(N₃-cytosine)Cl) (Bellon & Lippard, Biophys. Chem. 35(2-3):179-88, 1990), 3H-cis-1,2-diaminocyclohexanedichloroplatinum(II) and 3H-cis-1,2-diaminocyclohexane-malonatoplatinum (II) (Oswald et al., Res. Commun. Chem. Pathol. Pharmacol. 64(1):41-58, 1989), diaminocarboxylatoplatinum (EPA 296321), trans-(D,1)-1,2-diaminocyclohexane carrier ligand-bearing platinum analogues (Wyrick & Chaney, J. Labelled Compd. Radiopharm. 25(4):349-57, 1988), aminoalkylaminoanthraquinone-derived cisplatin analogues (Kitov et al., Eur. J. Med. Chem. 23(4):381-3, 1988), spiroplatin, carboplatin, iproplatin and JM40 platinum analogues (Schroyen et al., Eur. J. Cancer Clin. Oncol. 24(8):1309-12, 1988), bidentate tertiary diamine-containing cisplatinum derivatives (Orbell et al., Inorg. Chim. Acta 152(2):125-34, 1988), platinum(II), platinum(IV) (Liu & Wang, Shandong Yike Daxue Xuebao 24(1):35-41, 1986), cis-diammine(1,1-cyclobutanedicarboxylato-)platinum(II) (carboplatin, JM8) and ethylenediammine-malonatoplatinum(II) (JM40) (Begg et al., Radiother. Oncol. 9(2):157-65, 1987), JM8 and JM9 cisplatin analogues (Harstrick et al., Int. J. Androl. 10(1); 139-45, 1987), (NPr4)2((PtCL4).cis-(PtCl2-(NH2Me)2)) (Brammer et al., J. Chem. Soc., Chem. Commun. 6:443-5, 1987), aliphatic tricarboxylic acid platinum complexes (EPA 185225), cis-dichloro(amino acid )(tert-butylamine)platinum(II) complexes (Pasini & Bersanetti, Inorg. Chim. Acta 107(4):259-67, 1985); 4-hydroperoxycylcophosphamide (Ballard et al., Cancer Chemother. Pharmacol. 26(6):397-402, 1990), acyclouridine cyclophosphamide derivatives (Zakerinia et al., Helv. Chim. Acta 73(4):912-15, 1990), 1,3,2-dioxa- and -oxazaphosphorinane cyclophosphamide analogues (Yang et al., Tetrahedron 44(20):6305-14, 1988), C5-substituted cyclophosphamide analogues (Spada, University of Rhode Island Dissertation, 1987), tetrahydrooxazine cyclophosphamide analogues (Valente, University of Rochester Dissertation, 1988), phenyl ketone cyclophosphamide analogues (Hales et al., Teratology 39(1):31-7, 1989), phenylketophosphamide cyclophosphamide analogues (Ludeman et al., J. Med. Chem. 29(5):716-27, 1986), ASTA Z-7557 cyclophosphamide analogues (Evans et al., Int. J. Cancer 34(6):883-90, 1984), 3-(1-oxy-2,2,6,6-tetramethyl-4-piperidinyl)cyclophosphamide (Tsui et al., J. Med. Chem. 25(9):1106-10, 1982), 2-oxobis(2-β-chloroethylamino)-4-,6-dimethyl-1,3,2-oxazaphosphorinane cyclophosphamide (Carpenter et al., Phosphorus Sulfur 12(3):287-93, 1982), 5-fluoro- and 5-chlorocyclophosphamide (Foster et al., J. Med. Chem. 24(12):1399-403, 1981), cis- and trans-4-phenylcyclophosphamide (Boyd et al., J. Med. Chem. 23(4):372-5, 1980), 5-bromocyclophosphamide, 3,5-dehydrocyclophosphamide (Ludeman et al., J. Med. Chem. 22(2):151-8, 1979), 4-ethoxycarbonyl cyclophosphamide analogues (Foster, J. Pharm. Sci. 67(5):709-10, 1978), arylaminotetrahydro-2H-1,3,2-oxazaphosphorine 2-oxide cyclophosphamide analogues (Hamacher, Arch. Pharm. (Weinheim, Ger.) 310(5):J,428-34, 1977), NSC-26271 cyclophosphamide analogues (Montgomery & Struck, Cancer Treat. Rep. 60(4):J381-93, 1976), benzo annulated cyclophosphamide analogues (Ludeman & Zon, J. Med. Chem. 18(12):J1251-3, 1975), 6-trifluoromethylcyclophosphamide (Farmer& Cox, J. Med. Chem. 18(11):J1106-10, 1975), 4-methylcyclophosphamide and 6-methycyclophosphamide analogues (Cox et al., Biochem. Pharmacol. 24(5):J599-606, 1975); FCE 23762 doxorubicin derivative (Quaglia et al., J. Liq. Chromatogr. 17(18):3911-3923, 1994), annamycin (Zou et al., J. Pharm. Sci. 82(11):1151-1154, 1993), ruboxyl (Rapoport et al., J. Controlled Release 58(2):153-162, 1999), anthracycline disaccharide doxorubicin analogue (Pratesi et al., Clin. Cancer Res. 4(11):2833-2839, 1998), N-(trifluoroacetyl)doxorubicin and 4′-O-acetyl-N-(trifluoroacetyl)doxorubicin (Berube & Lepage, Synth. Commun. 28(6):1109-1116, 1998), 2-pyrrolinodoxorubicin (Nagy et al., Proc. Nat'l Acad. Sci. U.S.A. 95(4):1794-1799, 1998), disaccharide doxorubicin analogues (Arcamone et al., J. Nat'l Cancer Inst. 89(16):1217-1223, 1997), 4-demethoxy-7-O-(2,6-dideoxy-4-O-(2,3,6-trideoxy-3-amino-α-L-lyxo-hexopyranosyl)-α-L-lyxo-hexopyranosyl)-adriamicinone doxorubicin disaccharide analogue (Monteagudo et al., Carbohydr. Res. 300(1):11-16, 1997), 2-pyrrolinodoxorubicin (Nagy et al., Proc. Nat'l Acad. Sci. U.S.A. 94(2):652-656, 1997), morpholinyl doxorubicin analogues (Duran et al., Cancer Chemother. Pharmacol. 38(3):210-216, 1996), enaminomalonyl-β-alanine doxorubicin derivatives (Seitz et al., Tetrahedron Lett. 36(9):1413-16, 1995), cephalosporin doxorubicin derivatives (Vrudhula et al., J. Med. Chem. 38(8):1380-5, 1995), hydroxyrubicin (Solary et al., Int. J. Cancer 58(1):85-94, 1994), methoxymorpholino doxorubicin derivative (Kuhl et al., Cancer Chemother. Pharmacol. 33(1):10-16, 1993), (6-maleimidocaproyl)hydrazone doxorubicin derivative (Wiliner et al., Bioconjugate Chem. 4(6):521-7, 1993), N-(5,5-diacetoxypent-1-yl) doxorubicin (Cherif & Farquhar, J. Med. Chem. 35(17):3208-14, 1992), FCE 23762 methoxymorpholinyl doxorubicin derivative (Ripamonti et al., Br. J. Cancer 65(5):703-7, 1992), N-hydroxysuccinimide ester doxorubicin derivatives (Demant et al., Biochim. Biophys. Acta 1118(1):83-90, 1991), polydeoxynucleotide doxorubicin derivatives (Ruggiero et al., Biochim. Biophys. Acta 1129(3):294-302, 1991), morpholinyl doxorubicin derivatives (EPA 434960), mitoxantrone doxorubicin analogue (Krapcho et al., J. Med. Chem. 34(8):2373-80. 1991), AD198 doxorubicin analogue (Traganos et al., Cancer Res. 51(14):3682-9, 1991), 4-demethoxy-3′-N-trifluoroacetyldoxorubicin (Horton et al., Drug Des. Delivery 6(2):123-9, 1990), 4′-epidoxorubicin (Drzewoski et al., Pol. J. Pharmacol. Pharm. 40(2):159-65, 1988; Weenen et al., Eur. J. Cancer Clin. Oncol. 20(7):919-26, 1984), alkylating cyanomorpholino doxorubicin derivative (Scudder et al., J. Nat'l Cancer Inst. 80(16):1294-8, 1988), deoxydihydroiodooxorubicin (EPA 275966), adriblastin (Kalishevskaya et al., Vestn. Mosk. Univ., 16(Biol. 1):21-7, 1988), 4′-deoxydoxorubicin (Schoelzel et al., Leuk. Res. 10(12):1455-9, 1986), 4-demethyoxy-4′-o-methyldoxorubicin (Giuliani et al., Proc. Int. Congr. Chemother. 16:285-70-285-77, 1983), 3′-deamino-3′-hydroxydoxorubicin (Horton et al., J. Antibiot. 37(8):853-8, 1984), 4-demethyoxy doxorubicin analogues (Barbieri et al., Drugs Exp. Clin. Res. 10(2):85-90, 1984), N-L-leucyl doxorubicin derivatives (Trouet et al., Anthracyclines (Proc. Int. Symp. Tumor Pharmacother.), 179-81, 1983), 3′-deamino-3′-(4-methoxy-1-piperidinyl) doxorubicin derivatives (U.S. Pat. No. 4,314,054), 3′-deamino-3′-(4-mortholinyl) doxorubicin derivatives (U.S. Pat. No. 4,301,277), 4′-deoxydoxorubicin and 4′-o-methyidoxorubicin (Giuliani et al., Int. J. Cancer 27(1):5-13, 1981), aglycone doxorubicin derivatives (Chan & Watson, J. Pharm. Sci. 67(12):1748-52, 1978), SM 5887 (Pharma Japan 1468:20, 1995), MX-2 (Pharma Japan 1420:19, 1994), 4′-deoxy-13(S)-dihydro-4′-iododoxorubicin (EP 275966), morpholinyl doxorubicin derivatives (EPA 434960), 3′-deamino-3′-(4-methoxy-1-piperidinyl)doxorubicin derivatives (U.S. Pat. No. 4,314,054), doxorubicin-14-valerate, morpholinodoxorubicin (U.S. Pat. No. 5,004,606), 3′-deamino-3′-(3″-cyano-4″-morpholinyl doxorubicin; 3′-deamino-3′-(3″-cyano-4″-morpholinyl)-13-dihydoxorubicin; (3′-deamino-3′-(3″-cyano-4″-morpholinyl) daunorubicin; 3′-deamino-3′-(3″-cyano-4″-morpholinyl)-3-dihydrodaunorubicin; and 3′-deamino-3′-(4″-morpholinyl-5-iminodoxorubicin and derivatives (U.S. Pat. No. 4,585,859), 3′-deamino-3′-(4-methoxy-1-piperidinyl)doxorubicin derivatives (U.S. Pat. No. 4,314,054) and 3-deamino-3-(4-morpholinyl)doxorubicin derivatives (U.S. Pat. No. 4,301,277); 4,5-dimethylmisonidazole (Born et al., Biochem. Pharmacol. 43(6):1337-44, 1992), azo and azoxy misonidazole derivatives (Gaftavecchia & Tonelli, Int. J. Radiat Biol. Relat Stud. Phys., Chem. Med. 45(5):469-77, 1984); RB90740 (Wardman et al., Br. J. Cancer, 74 Suppl. (27):S70-S74, 1996); 6-bromo and 6-chloro-2,3-dihydro-1,4-benzothiazines nitrosourea derivatives (Rai et al., Heterocycl. Commun. 2(6):587-592, 1996), diamino acid nitrosourea derivatives (Dulude et al., Bioorg. Med. Chem. Lett. 4(22):2697-700, 1994; Dulude et al., Bioorg. Med. Chem. 3(2):151-60, 1995), amino acid nitrosourea derivatives (Zheleva et al., Pharmazie 50(1):25-6, 1995), 3′,4′-didemethoxy-3′,4′-dioxo-4-deoxypodophyllotoxin nitrosourea derivatives (Miyahara et al., Heterocycles 39(1):361-9, 1994), ACNU (Matsunaga et al., Immunopharmacology 23(3):199-204, 1992), tertiary phosphine oxide nitrosourea derivatives (Guguva et al., Pharmazie 46(8):603, 1991), sulfamerizine and sulfamethizole nitrosourea derivatives (Chiang et al., Zhonghua Yaozue Zazhi 43(5):401-6, 1991), thymidine nitrosourea analogues (Zhang et al., Cancer Commun. 3(4):119-26, 1991), 1,3-bis(2-chloroethyl)-1-nitrosourea (August et al., Cancer Res. 51(6):1586-90, 1991), 2,2,6,6-tetramethyl-1-oxopiperidiunium nitrosourea derivatives (U.S.S.R. 1261253), 2- and 4-deoxy sugar nitrosourea derivatives (U.S. Pat. No. 4,902,791), nitroxyl nitrosourea derivatives (U.S.S.R. 1336489), fotemustine (Boutin et al., Eur. J. Cancer Clin. Oncol. 25(9):1311-16, 1989), pyrimidine (II) nitrosourea derivatives (Wei et al., Chung-hua Yao Hsueh Tsa Chih 41(1):19-26, 1989), CGP 6809 (Schieweck et al., Cancer Chemother. Pharmacol. 23(6):341-7, 1989), B-3839 (Prajda et al., In Vivo 2(2):151-4, 1988), 5-halogenocytosine nitrosourea derivatives (Chiang & Tseng, T'ai-wan Yao Hsueh Tsa Chih 38(1):37-43, 1986), 1-(2-chloroethyl)-3-isobutyl-3-(β-maltosyl)-1-nitrosourea (Fujimoto & Ogawa, J. Pharmacobio-Dyn. 10(7):341-5, 1987), sulfur-containing nitrosoureas (Tang et al., Yaoxue Xuebao 21(7):502-9, 1986), sucrose, 6-((((2-chloroethyl)nitrosoamino-)carbonyl)amino)-6-deoxysucrose (NS-1C) and 6′-((((2-chloroethyl)nitrosoamino)carbonyl)amino)-6′-deoxysucrose (NS-1D) nitrosourea derivatives (Tanoh et al., Chemotherapy (Tokyo) 33(11):969-77, 1985), CNCC, RFCNU and chlorozotocin (Mena et al., Chemotherapy (Basel) 32(2):131-7, 1986), CNUA (Edanami et al., Chemotherapy (Tokyo) 33(5):455-61, 1985), 1-(2-chloroethyl)-3-isobutyl-3-(β-maltosyl)-1-nitrosourea (Fujimoto & Ogawa, Jpn. J. Cancer Res. (Gann) 76(7):651-6, 1985), choline-like nitrosoalkylureas (Belyaev et al., Izv. Akad. NAUK SSSR, Ser. Khim. 3:553-7, 1985), sucrose nitrosourea derivatives (JP 84219300), sulfa drug nitrosourea analogues (Chiang et al., Proc. Nat'l Sci. Counc., Repub. China, Part A 8(1):18-22, 1984), DONU (Asanuma et al., J. Jpn. Soc. Cancer Ther. 17(8):2035-43, 1982), N,N′-bis (N-(2-chloroethyl)-N-nitrosocarbamoyl)cystamine (CNCC) (Blazsek et al., Toxicol. Appl. Pharmacol. 74(2):250-7, 1984), dimethyinitrosourea (Krutova et al., Izv. Akad. NAUK SSSR, Ser. Biol. 3:439-45, 1984), GANU (Sava & Giraldi, Cancer Chemother. Pharmacol. 10(3):167-9, 1983), CCNU (Capelli et al., Med., Biol., Environ. 11(1):111-16, 1983), 5-aminomethyl-2′-deoxyuridine nitrosourea analogues (Shiau, Shih Ta Hsueh Pao (Taipei) 27:681-9, 1982), TA-077 (Fujimoto & Ogawa, Cancer Chemother. Pharmacol. 9(3):134-9, 1982), gentianose nitrosourea derivatives (JP 82 80396), CNCC, RFCNU, RPCNU AND chlorozotocin (CZT) (Marzin et al., INSERM Symp., 19(Nitrosoureas Cancer Treat.): 165-74, 1981), thiocolchicine nitrosourea analogues (George, Shih Ta Hsueh Pao (Taipei) 25:355-62, 1980), 2-chloroethyl-nitrosourea (Zeller & Eisenbrand, Oncology 38(1):39-42, 1981), ACNU, (1-(4-amino-2-methyl-5-pyrimidinyl)methyl-3-(2-chloroethyl)-3-nitrosourea hydrochloride) (Shibuya et al., Gan To Kagaku Ryoho 7(8):1393-401, 1980), N-deacetylmethyl thiocoichicine nitrosourea analogues (Lin et al., J. Med. Chem. 23(12):1440-2, 1980), pyridine and piperidine nitrosourea derivatives (Crider et al., J. Med. Chem. 23(8):848-51, 1980), methyl-CCNU (Zimber & Perk, Refu. Vet. 35(1):28, 1978), phensuzimide nitrosourea derivatives (Crider et al., J. Med. Chem. 23(3):324-6, 1980), ergoline nitrosourea derivatives (Crider et al., J. Med. Chem. 22(1):32-5, 1979), glucopyranose nitrosourea derivatives (JP 78 95917), 1-(2-chloroethyl)-3-cyclohexyl-1-nitrosourea (Farmer et al., J. Med. Chem. 21(6):514-20, 1978), 4-(3-(2-chloroethyl)-3-nitrosoureid-o)-cis-cyclohexanecarboxylic acid (Drewinko et al., Cancer Treat. Rep. 61(8):J1513-18, 1977), RPCNU (ICIG 1163) (Larnicol et al., Biomedicine 26(3):J176-81, 1977), IOB-252 (Sorodoc et al., Rev. Roum. Med., Virol. 28(1):J 55-61, 1977), 1,3-bis(2-chloroethyl)-1-nitrosourea (BCNU) (Siebert & Eisenbrand, Mutat. Res. 42(1):J45-50, 1977), 1-tetrahydroxycyclopentyl-3-nitroso-3-(2-chloroethyl)-urea (U.S. Pat. No. 4,039,578), d-1-1-(β-chloroethyl)-3-(2-oxo-3-hexahydroazepinyl)-1-nitrosourea (U.S. Pat. No. 3,859,277) and gentianose nitrosourea derivatives (JP 57080396); 6-S-aminoacyloxymethyl mercaptopurine derivatives (Harada et al., Chem. Pharm. Bull. 43(10):793-6, 1995), 6-mercaptopurine (6-MP) (Kashida et al., Biol. Pharm. Bull. 18(11):1492-7, 1995), 7,8-polymethyleneimidazo-1,3,2-diazaphosphorines (Nilov et al., Mendeleev Commun. 2:67, 1995), azathioprine (Chifotides et al., J. Inorg. Biochem. 56(4):249-64, 1994), methyl-D-glucopyranoside mercaptopurine derivatives (Da Silva et al., Eur. J. Med. Chem. 29(2):149-52, 1994) and s-alkynyl mercaptopurine derivatives (Ratsino et al., Khim.-Farm. Zh. 15(8):65-7, 1981); indoline ring and a modified ornithine or glutamic acid-bearing methotrexate derivatives (Matsuoka et al., Chem. Pharm. Bull. 45(7):1146-1150, 1997), alkyl-substituted benzene ring C bearing methotrexate derivatives (Matsuoka et al., Chem. Pharm. Bull. 44(12):2287-2293, 1996), benzoxazine or benzothiazine moiety-bearing methotrexate derivatives (Matsuoka et al., J. Med. Chem. 40(1):105-111, 1997), 10-deazaaminopterin analogues (DeGraw et al., J. Med. Chem. 40(3):370-376, 1997), 5-deazaaminopterin and 5,10-dideazaaminopterin methotrexate analogues (Piper et al., J. Med. Chem. 40(3):377-384, 1997), indoline moiety-bearing methotrexate derivatives (Matsuoka et al., Chem. Pharm. Bull. 44(7):1332-1337, 1996), lipophilic amide methotrexate derivatives (Pignatello et al., World Meet. Pharm., Biopharm. Pharm. Technol., 563-4, 1995), L-threo-(2S, 4S)-4-fluoroglutamic acid and DL-3,3-difluoroglutamic acid-containing methotrexate analogues (Hart et al., J. Med. Chem. 39(1):56-65, 1996), methotrexate tetrahydroquinazoline analogue (Gangjee, et al., J. Heterocycl. Chem. 32(1):243-8, 1995), N-(α-aminoacyl)methotrexate derivatives (Cheung et al., Pteridines 3(1-2):101-2, 1992), biotin methotrexate derivatives (Fan et al., Pteridines 3(1-2):131-2, 1992), D-glutamic acid or D-erythrou, threo-4-fluoroglutamic acid methotrexate analogues (McGuire et al., Biochem. Pharmacol. 42(12):2400-3, 1991), β,γ-methano methotrexate analogues (Rosowsky et al., Pteridines 2(3):133-9, 1991), 10-deazaaminopterin (10-EDAM) analogue (Braakhuis et al., Chem. Biol. Pteridines, Proc. Int. Symp. Pteridines Folic Acid Deriv., 1027-30, 1989), γ-tetrazole methotrexate analogue (Kalman et al., Chem. Biol. Pteridines, Proc. Int. Symp. Pteridines Folic Acid Deriv., 1154-7, 1989), N-(L-α-aminoacyl)methotrexate derivatives (Cheung et al., Heterocycles 28(2):751-8, 1989), meta and ortho isomers of aminopterin (Rosowsky et al., J. Med. Chem. 32(12):2582, 1989), hydroxymethylmethotrexate (DE 267495), γ-fluoromethotrexate (McGuire et al., Cancer Res. 49(16):4517-25, 1989), polyglutamyl methotrexate derivatives (Kumar et al., Cancer Res. 46(10):5020-3, 1986), gem-diphosphonate methotrexate analogues (WO 88/06158), α- and γ-substituted methotrexate analogues (Tsushima et al., Tetrahedron 44(17):5375-87, 1988), 5-methyl-5-deaza methotrexate analogues (U.S. Pat. No. 4,725,687), N6-acyl-Nα-(4-amino-4-deoxypteroyl)-L-ornithine derivatives (Rosowsky et al., J. Med. Chem. 31(7):1332-7, 1988), 8-deaza methotrexate analogues (Kuehl et al., Cancer Res. 48(6):1481-8, 1988), acivicin methotrexate analogue (Rosowsky et al., J. Med. Chem. 30(8):1463-9, 1987), polymeric platinol methotrexate derivative (Carraher et al., Polym. Sci. Technol. (Plenum), 35(Adv. Biomed. Polym.):311-24, 1987), methotrexate-γ-dimyristoylphophatidylethanolamine (Kinsky et a l., Biochim. Biophys. Acta 917(2):211-18, 1987), methotrexate polyglutamate analogues (Rosowsky et al., Chem. Biol. Pteridines, Pteridines Folic Acid Deriv., Proc. Int. Symp. Pteridines Folic Acid Deriv.: Chem., Biol. Clin. Aspects: 985-8, 1986), poly-γ-glutamyl methotrexate derivatives (Kisliuk et al., Chem. Biol. Pteridines, Pteridines Folic Acid Deriv., Proc. Int. Symp. Pteridines Folic Acid Deriv.: Chem., Biol. Clin. Aspects: 989-92, 1986), deoxyuridylate methotrexate derivatives (Webber et al., Chem. Biol. Pteridines, Pteridines Folic Acid Deriv., Proc. Int. Symp. Pteridines Folic Acid Deriv.: Chem., Biol. Clin. Aspects: 659-62, 1986), iodoacetyl lysine methotrexate analogue (Delcamp et al., Chem. Biol. Pteridines, Pteridines Folic Acid Deriv., Proc. Int. Symp. Pteridines Folic Acid Deriv.: Chem., Biol. Clin. Aspects: 807-9, 1986), 2,.omega.-diaminoalkanoid acid-containing methotrexate analogues (McGuire et al., Biochem. Pharmacol. 35(15):2607-13, 1986), polyglutamate methotrexate derivatives (Kamen & Winick, Methods Enzymol. 122 (Vitam. Coenzymes, Pt. G):339-46, 1986), 5-methyl-5-deaza analogues (Piper et al., J. Med. Chem. 29(6):1080-7, 1986), quinazoline methotrexate analogue (Mastropaolo et al., J. Med. Chem. 29(1):155-8, 1986), pyrazine methotrexate analogue (Lever & Vestal, J. Heterocycl. Chem. 22(1):5-6, 1985), cysteic acid and homocysteic acid methotrexate analogues (4,490,529), γ-tert-butyl methotrexate esters (Rosowsky et al., J. Med. Chem. 28(5):660-7, 1985), fluorinated methotrexate analogues (Tsushima et al., Heterocycles 23(1):45-9, 1985), folate methotrexate analogue (Trombe, J. Bacteriol. 160(3):849-53, 1984), phosphonoglutamic acid analogues (Sturtz & Guillamot, Eur. J. Med. Chem.—Chim. Ther. 19(3):267-73, 1984), poly(L-lysine)methotrexate conjugates (Rosowsky et al., J. Med. Chem. 27(7):888-93, 1984), dilysine and trilysine methotrexate derivates (Forsch & Rosowsky, J. Org. Chem. 49(7):1305-9, 1984), 7-hydroxymethotrexate (Fabre et al., Cancer Res. 43(10):4648-52, 1983), poly-γ-glutamyl methotrexate analogues (Piper & Montgomery, Adv. Exp. Med. Biol., 163(Folyl Antifolyl Polyglutamates):95-100, 1983), 3′,5′-dichloromethotrexate (Rosowsky & Yu, J. Med. Chem. 26(10):1448-52, 1983), diazoketone and chloromethylketone methotrexate analogues (Gangjee et al., J. Pharm. Sci. 71(6):717-19, 1982), 10-propargylaminopterin and alkyl methotrexate homologs (Piper et al., J. Med. Chem. 25(7):877-80, 1982), lectin derivatives of methotrexate (Lin et al., JNCI 66(3):523-8, 1981), polyglutamate methotrexate derivatives (Galivan, Mol. Pharmacol. 17(1):105-10, 1980), halogentated methotrexate derivatives (Fox, JNCI 58(4):J955-8, 1977), 8-alkyl-7,8-dihydro analogues (Chaykovsky et al., J. Med. Chem. 20(10):J1323-7, 1977), 7-methyl methotrexate derivatives and dichloromethotrexate (Rosowsky & Chen, J. Med. Chem. 17(12):J1308-11, 1974), lipophilic methotrexate derivatives and 3′,5′-dichloromethotrexate (Rosowsky, J. Med. Chem. 16(10):J1190-3, 1973), deaza amethopterin analogues (Montgomery et al., Ann. N.Y. Acad. Sci. 186:J227-34, 1971), MX068 (Pharma Japan, 1658:18, 1999) and cysteic acid and homocysteic acid methotrexate analogues (EPA 0142220); N3-alkylated analogues of 5-fluorouracil (Kozai et al., J. Chem. Soc., Perkin Trans. 1(19):3145-3146, 1998), 5-fluorouracil derivatives with 1,4-oxaheteroepane moieties (Gomez et al., Tetrahedron 54(43):13295-13312, 1998), 5-fluorouracil and nucleoside analogues (Li, Anticancer Res. 17(1A):21-27, 1997), cis- and trans-5-fluoro-5,6-dihydro-6-alkoxyuracil (Van der Wilt et al., Br. J. Cancer 68(4):702-7, 1993), cyclopentane 5-fluorouracil analogues (Hronowski & Szarek, Can. J. Chem. 70(4):1162-9, 1992), A-OT-fluorouracil (Zhang et al., Zongguo Yiyao Gongye Zazhi 20(11):513-15, 1989), N4-trimethoxybenzoyl-5′-deoxy-5-fluorocytidine and 5′-deoxy-5-fluorouridine (Miwa et al., Chem. Pharm. Bull. 38(4):998-1003, 1990), 1-hexylcarbamoyl-5-fluorouracil (Hoshi et al., J. Pharmacobio-Dun. 3(9):478-81, 1980; Maehara et al., Chemotherapy (Basel) 34(6):484-9, 1988), B-3839 (Prajda et al., In Vivo 2(2):151-4, 1988), uracil-1-(2-tetrahydrofuryl)-5-fluorouracil (Anai et al., Oncology 45(3):144-7, 1988), 1-(2′-deoxy-2′-fluoro-β-D-arabinofuranosyl)-5-fluorouracil (Suzuko et al., Mol. Pharmacol. 31(3):301-6, 1987), doxifluridine (Matuura et al., Oyo Yakuri 29(5):803-31, 1985), 5′-deoxy-5-fluorouridine (Bollag & Hartmann, Eur. J. Cancer 16(4):427-32, 1980), 1-acetyl-3-O-toluyl-5-fluorouracil (Okada, Hiroshima J. Med. Sci. 28(1):49-66, 1979), 5-fluorouracil-m-formylbenzene-sulfonate (JP 55059173), N′-(2-furanidyl)-5-fluorouracil (JP 53149985) and 1-(2-tetrahydrofuryl)-5-fluorouracil (JP 52089680); 4′-epidoxorubicin (Lanius, Adv. Chemother. Gastrointest. Cancer, (Int. Symp.), 159-67, 1984); N-substituted deacetylvinblastine amide (vindesine) sulfates (Conrad et al., J. Med. Chem. 22(4):391-400, 1979); and Cu(II)-VP-16 (etoposide) complex (Tawa et al., Bioorg. Med. Chem. 6(7):1003-1008, 1998), pyrrolecarboxamidino-bearing etoposide analogues (Ji et al., Bioorg. Med. Chem. Lett. 7(5):607-612, 1997), 4βp-amino etoposide analogues (Hu, University of North Carolina Dissertation, 1992), γ-lactone ring-modified arylamino etoposide analogues (Zhou et al., J. Med. Chem. 37(2):287-92, 1994), N-glucosyletoposide analogue (Allevi et al., Tetrahedron Lett. 34(45):7313-16, 1993), etoposide A-ring analogues (Kadow et al., Bioorg. Med. Chem. Lett. 2(1):17-22, 1992), 4′-deshydroxy-4′-methyl etoposide (Saulnier et al., Bioorg. Med. Chem. Lett. 2(10):1213-18, 1992), pendulum ring etoposide analogues (Sinha et al., Eur. J. Cancer 26(5):590-3, 1990) and E-ring desoxy etoposide analogues (Saulnier et al., J. Med. Chem. 32(7):1418-20, 1989).

Within one embodiment of the invention, the cell cycle inhibitor is paclitaxel, a compound which disrupts mitosis (M-phase) by binding to tubulin to form abnormal mitotic spindles or an analogue or derivative thereof. Briefly, paclitaxel is a highly derivatized diterpenoid (Wani et al., J. Am. Chem. Soc. 93:2325, 1971) which has been obtained from the harvested and dried bark of Taxus brevifolia (Pacific Yew) and Taxomyces Andreanae and Endophytic Fungus of the Pacific Yew (Stierle et al., Science 60:214-216, 1993). “Paclitaxel” (which should be understood herein to include formulations, prodrugs, analogues and derivatives such as, for example, TAXOL (Bristol Myers Squibb, New York, N.Y., TAXOTERE (Aventis Pharmaceuticals, France), docetaxel, 10-desacetyl analogues of paclitaxel and 3′N-desbenzoyl-3′N-t-butoxy carbonyl analogues of paclitaxel) may be readily prepared utilizing techniques known to those skilled in the art (see, e.g., Schiff et al., Nature 277:665-667, 1979; Long and Fairchild, Cancer Research 54:4355-4361, 1994; Ringel and Horwitz, J. Nat'l Cancer Inst. 83(4):288-291, 1991; Pazdur et al., Cancer Treat Rev. 19(4):351-386, 1993; WO 94/07882; WO 94/07881; WO 94/07880; WO 94/07876; WO 93/23555; WO 93/10076; W094/00156; WO 93/24476; EP 590267; WO 94/20089; U.S. Pat. Nos. 5,294,637; 5,283,253; 5,279,949; 5,274,137; 5,202,448; 5,200,534; 5,229,529; 5,254,580; 5,412,092; 5,395,850; 5,380,751; 5,350,866; 4,857,653; 5,272,171; 5,411,984; 5,248,796; 5,248,796; 5,422,364; 5,300,638; 5,294,637; 5,362,831; 5,440,056; 4,814,470; 5,278,324; 5,352,805; 5,411,984; 5,059,699; 4,942,184; Tetrahedron Letters 35(52):9709-9712, 1994; J. Med. Chem. 35:4230-4237, 1992; J. Med. Chem. 34:992-998, 1991; J. Natural Prod. 57(10):1404-1410, 1994; J. Natural Prod. 57(11):1580-1583, 1994; J. Am. Chem. Soc. 110:6558-6560, 1988), or obtained from a variety of commercial sources, including for example, Sigma Chemical Co., St. Louis, Mo. (T7402—from Taxus brevifolia).

Representative examples of paclitaxel derivatives or analogues include 7-deoxy-docetaxol, 7,8-cyclopropataxanes, N-substituted 2-azetidones, 6,7-epoxy paclitaxels, 6,7-modified paclitaxels, 10-desacetoxytaxol, 10-deacetyltaxol (from 10-deacetylbaccatin III), phosphonooxy and carbonate derivatives of taxol, taxol 2′,7-di(sodium 1,2-benzenedicarboxylate, 10-desacetoxy-11,12-dihydrotaxol-10,12(18)-diene derivatives, 10-desacetoxytaxol, Protaxol (2′- and/or 7-O-ester derivatives), (2′-and/or 7-O-carbonate derivatives), asymmetric synthesis of taxol side chain, fluoro taxols, 9-deoxotaxane, (13-acetyl-9-deoxobaccatine III, 9-deoxotaxol, 7-deoxy-9-deoxotaxol, 10-desacetoxy-7-deoxy-9-deoxotaxol, Derivatives containing hydrogen or acetyl group and a hydroxy and tert-butoxycarbonylamino, sulfonated 2′-acryloyltaxol and sulfonated 2′-O-acyl acid taxol derivatives, succinyltaxol, 2′-γ-aminobutyryltaxol formate, 2′-acetyl taxol, 7-acetyl taxol, 7-glycine carbamate taxol, 2′-OH-7-PEG(5000) carbamate taxol, 2′-benzoyl and 2′,7-dibenzoyl taxol derivatives, other prodrugs (2′-acetyltaxol; 2′,7-diacetyltaxol; 2′succinyltaxol; 2′-(beta-alanyl)-taxol); 2′gamma-aminobutyryltaxol formate; ethylene glycol derivatives of 2′-succinyltaxol; 2′-glutaryltaxol; 2′-(N,N-dimethylglycyl)taxol; 2′-(2-(N,N-dimethylamino)propionyl)taxol; 2′orthocarboxybenzoyl taxol; 2′aliphatic carboxylic acid derivatives of taxol, Prodrugs {2′(N,N-diethylaminopropionyl)taxol, 2′(N,N-dimethylglycyl)taxol, 7(N,N-dimethylglycyl)taxol, 2′,7-di-(N,N-dimethylglycyl)taxol, 7(N,N-diethylaminopropionyl)taxol, 2′,7-di(N,N-diethylaminopropionyl)taxol, 2′-(L-glycyl)taxol, 7-(L-glycyl)taxol, 2′,7-di(L-glycyl)taxol, 2′-(L-alanyl)taxol, 7-(L-alanyl)taxol, 2′,7-di(L-alanyl)taxol, 2′-(L-leucyl)taxol, 7-(L-leucyl)taxol, 2′,7-di(L-leucyl)taxol, 2′-(L-isoleucyl)taxol, 7-(L-isoleucyl)taxol, 2′,7-di(L-isoleucyl)taxol, 2′-(L-valyl)taxol, 7-(L-valyl)taxol, 2′7-di(L-valyl)taxol, 2′-(L-phenylalanyl)taxol, 7-(L-phenylalanyl)taxol, 2′,7-di(L-phenylalanyl)taxol, 2′-(L-prolyl)taxol, 7-(L-prolyl)taxol, 2′,7-di(L-prolyl)taxol, 2′-(L-lysyl)taxol, 7-(L-lysyl)taxol, 2′,7-di(L-lysyl)taxol, 2′-(L-glutamyl)taxol, 7-(L-glutamyl)taxol, 2′,7-di(L-glutamyl)taxol, 2′-(L-arginyl)taxol, 7-(L-arginyl)taxol, 2′,7-di(L-arginyl)taxol}, taxol analogues with modified phenylisoserine side chains, TAXOTERE, (N-debenzoyl-N-tert-(butoxycaronyl)-10-deacetyltaxol, and taxanes (e.g., baccatin III, cephalomannine, 10-deacetylbaccatin III, brevifoliol, yunantaxusin and taxusin); and other taxane analogues and derivatives, including 14-beta-hydroxy-10 deacetybaccatin III, debenzoyl-2-acyl paclitaxel derivatives, benzoate paclitaxel derivatives, phosphonooxy and carbonate paclitaxel derivatives, sulfonated 2′-acryloyltaxol; sulfonated 2′-O-acyl acid paclitaxel derivatives, 18-site-substituted paclitaxel derivatives, chlorinated paclitaxel analogues, C4 methoxy ether paclitaxel derivatives, sulfenamide taxane derivatives, brominated paclitaxel analogues, Girard taxane derivatives, nitrophenyl paclitaxel, 10-deacetylated substituted paclitaxel derivatives, 14-beta-hydroxy-10 deacetylbaccatin III taxane derivatives, C7 taxane derivatives, C10 taxane derivatives, 2-debenzoyl-2-acyl taxane derivatives, 2-debenzoyl and -2-acyl paclitaxel derivatives, taxane and baccatin III analogues bearing new C2 and C4 functional groups, n-acyl paclitaxel analogues, 10-deacetylbaccatin III and 7-protected-10-deacetylbaccatin III derivatives from 10-deacetyl taxol A, 10-deacetyl taxol B, and 10-deacetyl taxol, benzoate derivatives of taxol, 2-aroyl-4-acyl paclitaxel analogues, orthro-ester paclitaxel analogues, 2-aroyl-4-acyl paclitaxel analogues and 1-deoxy paclitaxel and 1-deoxy paclitaxel analogues.

In one aspect, the cell cycle inhibitor is a taxane having the formula (C1): 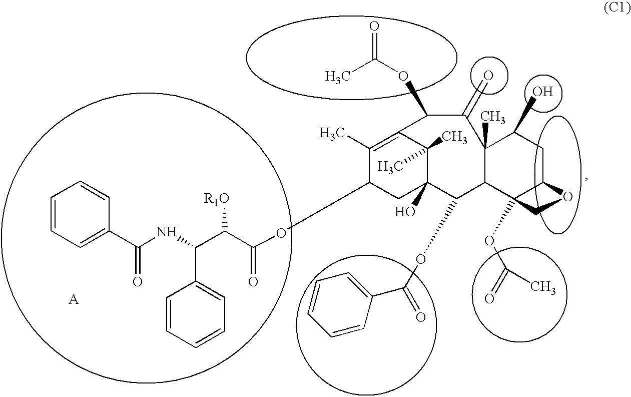
where the gray-highlighted portions may be substituted and the non-highlighted portion is the taxane core. A side-chain (labeled “A” in the diagram) is desirably present in order for the compound to have good activity as a cell cycle inhibitor. Examples of compounds having this structure include paclitaxel (Merck Index entry 7117), docetaxol (TAXOTERE, Merck Index entry 3458), and 3′-desphenyl-3′-(4-ntirophenyl)-N-debenzoyl-N-(t-butoxycarbonyl)-10-deacetyltaxol.

In one aspect, suitable taxanes such as paclitaxel and its analogues and derivatives are disclosed in U.S. Pat. No. 5,440,056 as having the structure (C2): 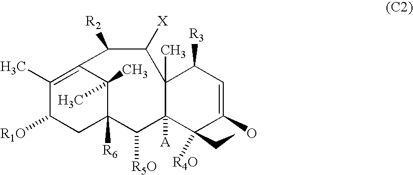
wherein X may be oxygen (paclitaxel), hydrogen (9-deoxy derivatives), thioacyl, or dihydroxyl precursors; R₁ is selected from paclitaxel or TAXOTERE side chains or alkanoyl of the formula (C3) 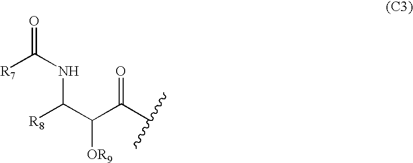
wherein R₇ is selected from hydrogen, alkyl, phenyl, alkoxy, amino, phenoxy (substituted or unsubstituted); R₈ is selected from hydrogen, alkyl, hydroxyalkyl, alkoxyalkyl, aminoalkyl, phenyl (substituted or unsubstituted), alpha or beta-naphthyl; and R₉ is selected from hydrogen, alkanoyl, substituted alkanoyl, and aminoalkanoyl; where substitutions refer to hydroxyl, sulfhydryl, allalkoxyl, carboxyl, halogen, thioalkoxyl, N,N-dimethylamino, alkylamino, dialkylamino, nitro, and —OSO₃H, and/or may refer to groups containing such substitutions; R₂ is selected from hydrogen or oxygen-containing groups, such as hydrogen, hydroxyl, alkoyl, alkanoyloxy, aminoalkanoyloxy, and peptidyalkanoyloxy; R₃ is selected from hydrogen or oxygen-containing groups, such as hydrogen, hydroxyl, alkoyl, alkanoyloxy, aminoalkanoyloxy, and peptidyalkanoyloxy, and may further be a silyl containing group or a sulphur containing group; R₄ is selected from acyl, alkyl, alkanoyl, aminoalkanoyl, peptidylalkanoyl and aroyl; R₅ is selected from acyl, alkyl, alkanoyl, aminoalkanoyl, peptidylalkanoyl and aroyl; R₆ is selected from hydrogen or oxygen-containing groups, such as hydrogen, hydroxyl alkoyl, alkanoyloxy, aminoalkanoyloxy, and peptidyalkanoyloxy.

In one aspect, the paclitaxel analogues and derivatives useful as cell cycle inhibitors are disclosed in PCT International Patent Application No. WO 93/10076. As disclosed in this publication, the analogue or derivative should have a side chain attached to the taxane nucleus at C₁₃, as shown in the structure below (formula C4), in order to confer antitumor activity to the taxane.

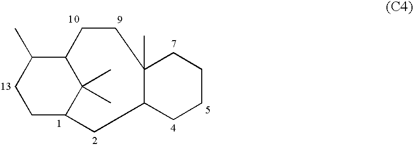

WO 93/10076 discloses that the taxane nucleus may be substituted at any position with the exception of the existing methyl groups. The substitutions may include, for example, hydrogen, alkanoyloxy, alkenoyloxy, aryloyloxy. In addition, oxo groups may be attached to carbons labeled 2, 4, 9, and/or 10. As well, an oxetane ring may be attached at carbons 4 and 5. As well, an oxirane ring may be attached to the carbon labeled 4.

In one aspect, the taxane-based cell cycle inhibitor useful in the present invention is disclosed in U.S. Pat. No. 5,440,056, which discloses 9-deoxo taxanes. These are compounds lacking an oxo group at the carbon labeled 9 in the taxane structure shown above (formula C4). The taxane ring may be substituted at the carbons labeled 1, 7 and 10 (independently) with H, OH, O—R, or O—CO—R where R is an alkyl or an aminoalkyl. As well, it may be substituted at carbons labeled 2 and 4 (independently) with aryol, alkanoyl, aminoalkanoyl or alkyl groups. The side chain of formula (C3) may be substituted at R₇ and R₈ (independently) with phenyl rings, substituted phenyl rings, linear alkanes/alkenes, and groups containing H, O or N. R₉ may be substituted with H, or a substituted or unsubstituted alkanoyl group.

Taxanes in general, and paclitaxel is particular, is considered to function as a cell cycle inhibitor by acting as an anti-microtubule agent, and more specifically as a stabilizer. These compounds have been shown useful in the treatment of proliferative disorders, including: non-small cell (NSC) lung; small cell lung; breast; prostate; cervical; endometrial; head and neck cancers.

In another aspect, the anti-microtuble agent (microtubule inhibitor) is albendazole (carbamic acid, (5-(propylthio)-1H-benzimidazol-2-yl)-, methyl ester), LY-355703 (1,4-dioxa-8,11-diazacyclohexadec-13-ene-2,5,9,12-tetrone, 10-((3-chloro-4-methoxyphenyl)methyl)-6,6-dimethyl-3-(2-methylpropyl)-16-((1S)-1-((2S,3R)-3-phenyloxiranyl)ethyl)-, (3S,10R,13E,16S)-), vindesine (vincaleukoblastine, 3-(aminocarbonyl)-O4-deacetyl-3-de(methoxycarbonyl)-), or WAY-174286

In another aspect, the cell cycle inhibitor is a vinca alkaloid. Vinca alkaloids have the following general structure. They are indole-dihydroindole dimers.

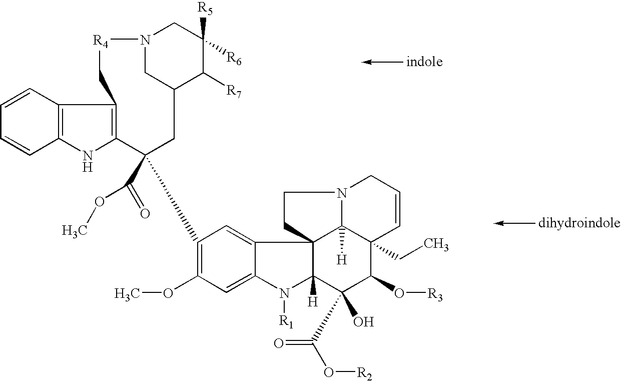

As disclosed in U.S. Pat. Nos. 4,841,045 and 5,030,620, R₁ can be a formyl or methyl group or alternately H. R₁ can also be an alkyl group or an aldehyde-substituted alkyl (e.g., CH₂CHO). R₂ is typically a CH₃ or NH₂ group. However it can be alternately substituted with a lower alkyl ester or the ester linking to the dihydroindole core may be substituted with C(O)—R where R is NH₂, an amino acid ester or a peptide ester. R₃ is typically C(O)CH₃, CH₃ or H. Alternately, a protein fragment may be linked by a bifunctional group, such as maleoyl amino acid. R₃ can also be substituted to form an alkyl ester which may be further substituted. R₄ may be —CH₂— or a single bond. R₅ and R₆ may be H, OH or a lower alkyl, typically —CH₂CH₃. Alternatively R₆ and R₇ may together form an oxetane ring. R₇ may alternately be H. Further substitutions include molecules wherein methyl groups are substituted with other alkyl groups, and whereby unsaturated rings may be derivatized by the addition of a side group such as an alkane, alkene, alkyne, halogen, ester, amide or amino group.

Exemplary vinca alkaloids are vinblastine, vincristine, vincristine sulfate, vindesine, and vinorelbine, having the structures:

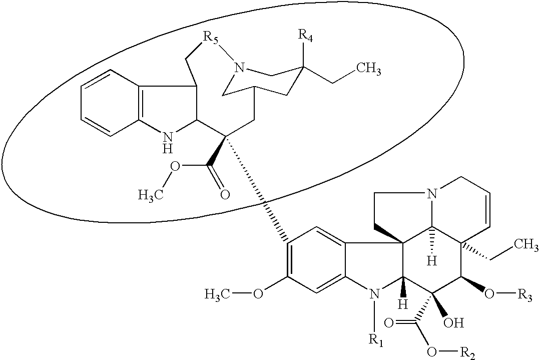

| | R₁ | R₂ | R₃ | R₄ | R₅ |
|---|---|---|---|---|---|
| Vinblastine: | CH₃ | CH₃ | C(O)CH₃ | OH | CH₂ |
| Vincristine: | CH₂O | CH₃ | C(O)CH₃ | OH | CH₂ |
| Vindesine: | CH₃ | NH₂ | H | OH | CH₂ |
| Vinorelbine: | CH₃ | CH₃ | CH₃ | H | single bond |

Analogues typically require the side group (shaded area) in order to have activity. These compounds are thought to act as cell cycle inhibitors by functioning as anti-microtubule agents, and more specifically to inhibit polymerization. These compounds have been shown useful in treating proliferative disorders, including NSC lung; small cell lung; breast; prostate; brain; head and neck; retinoblastoma; bladder; and penile cancers; and soft tissue sarcoma.

In another aspect, the cell cycle inhibitor is a camptothecin, or an analog or derivative thereof. Camptothecins have the following general structure.

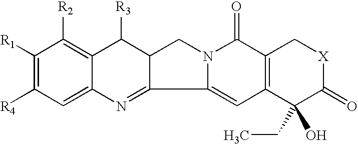

In this structure, X is typically O, but can be other groups, e.g., NH in the case of 21-lactam derivatives. R₁ is typically H or OH, but may be other groups, e.g., a terminally hydroxylated C₁₋₃ alkane. R₂ is typically H or an amino containing group such as (CH₃)₂NHCH₂, but may be other groups e.g., NO₂, NH₂, halogen (as disclosed in, e.g., U.S. Pat. No. 5,552,156) or a short alkane containing these groups. R₃ is typically H or a short alkyl such as C₂H₅. R₄ is typically H but may be other groups, e.g., a methylenedioxy group with R₁.

Exemplary camptothecin compounds include topotecan, irinotecan (CPT-11), 9-aminocamptothecin, 21-lactam-20(S)-camptothecin, 10,11-methylenedioxycamptothecin, SN-38, 9-nitrocamptothecin, 10-hydroxycamptothecin. Exemplary compounds have the structures:

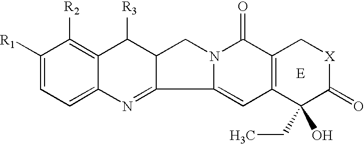

| | R₁ | R₂ | R₃ |
|---|---|---|---|
| Camptothecin: | H | H | H |
| Topotecan: | OH | (CH₃)₂NHCH₂ | H |
| SN-38: | OH | H | C₂H₅ |

X: O for most analogs, NH for 21-lactam analogs

Camptothecins have the five rings shown here. The ring labeled E must be intact (the lactone rather than carboxylate form) for maximum activity and minimum toxicity. These compounds are useful to as cell cycle inhibitors, where they can function as topoisomerase I inhibitors and/or DNA cleavage agents. They have been shown useful in the treatment of proliferative disorders, including, for example, NSC lung; small cell lung; and cervical cancers.

In another aspect, the cell cycle inhibitor is a podophyllotoxin, or a derivative or an analogue thereof. Exemplary compounds of this type are etoposide or teniposide, which have the following structures:

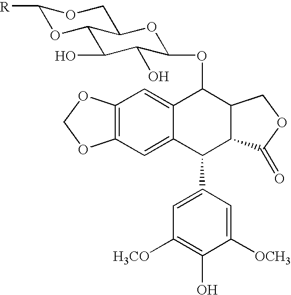

| | R |
|---|---|
| Etoposide | CH₃ |
| Teniposide | 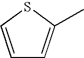 |

These compounds are thought to function as cell cycle inhibitors by being topoisomerase II inhibitors and/or by DNA cleaving agents. They have been shown useful as antiproliferative agents in, e.g., small cell lung, prostate, and brain cancers, and in retinoblastoma.

Another example of a DNA topoisomerase inhibitor is lurtotecan dihydrochloride (11H-1,4-dioxino(2,3-g)pyrano(3′,4′:6,7)indolizino(1,2-b)quinoline-9,12(8H,14H)-dione, 8-ethyl-2,3-dihydro-8-hydroxy-15-((4-methyl-1-piperazinyl)methyl)-, dihydrochloride, (S)-).

In another aspect, the cell cycle inhibitor is an anthracycline. Anthracyclines have the following general structure, where the R groups may be a variety of organic groups:

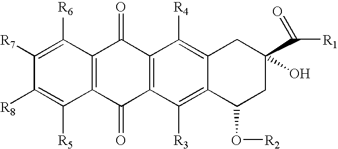

According to U.S. Pat. No. 5,594,158, suitable R groups are: R₁ is CH₃ or CH₂OH; R₂ is daunosamine or H; R₃ and R₄ are independently one of OH, NO₂, NH₂, F, Cl, Br, I, CN, H or groups derived from these; R₅₋₇ are all H or R₅ and R₆ are H and R₇ and R₈ are alkyl or halogen, or vice versa: R₇ and R₈ are H and R₅ and R₆ are alkyl or halogen.

According to U.S. Pat. No. 5,843,903, R₂ may be a conjugated peptide. According to U.S. Pat. Nos. 4,215,062 and 4,296,105, R₅ may be OH or an ether linked alkyl group. R₁ may also be linked to the anthracycline ring by a group other than C(O), such as an alkyl or branched alkyl group having the C(O) linking moiety at its end, such as —CH₂CH(CH₂—X)C(O)—R₁, wherein X is H or an alkyl group (see, e.g., U.S. Pat. No. 4,215,062). R₂ may alternately be a group linked by the functional group ═N—NHC(O)—Y, where Y is a group such as a phenyl or substituted phenyl ring. Alternately R₃ may have the following structure: 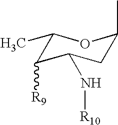
in which R₉ is OH either in or out of the plane of the ring, or is a second sugar moiety such as R₃. R₁₀ may be H or form a secondary amine with a group such as an aromatic group, saturated or partially saturated 5 or 6 membered heterocyclic having at least one ring nitrogen (see U.S. Pat. No. 5,843,903). Alternately, R₁₀ may be derived from an amino acid, having the structure —C(O)CH(NHR₁₁)(R₁₂), in which R₁₁ is H, or forms a C₃₋₄ membered alkylene with R₁₂. R₁₂ may be H, alkyl, aminoalkyl, amino, hydroxy, mercapto, phenyl, benzyl or methylthio (see U.S. Pat. No. 4,296,105).

Exemplary anthracyclines are doxorubicin, daunorubicin, idarubicin, epirubicin, pirarubicin, zorubicin, and carubicin. Suitable compounds have the structures:

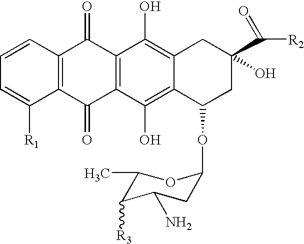

| | R₁ | R₂ | R₃ |
|---|---|---|---|
| Doxorubicin: | OCH₃ | CH₂OH | OH out of ring plane |
| Epirubicin: | OCH₃ | CH₂OH | OH in ring plane |
| (4′ epimer | | | |
| of doxorubicin) | | | |
| Daunorubicin: | OCH₃ | CH₃ | OH out of ring plane |
| Idarubicin: | H | CH₃ | OH out of ring plane |
| Pirarubicin | OCH₃ | OH | A |
| Zorubicin | OCH₃ | ═N—NHC(O)C₆H₅ | B |
| Carubicin | O | CH₃ | B |

A:

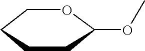

B:

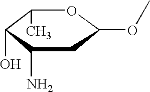

Other suitable anthracyclines are anthramycin, mitoxantrone, menogaril, nogalamycin, aclacinomycin A, olivomycin A, chromomycin A₃, and plicamycin having the structures:

Anthramycin

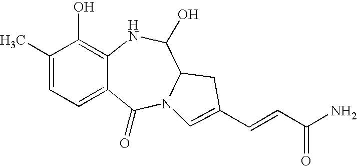

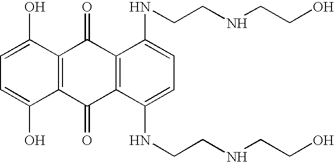

Mitoxantrone

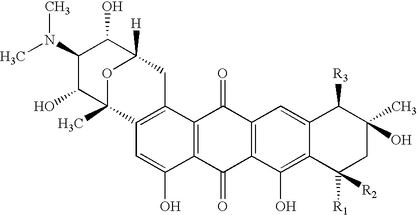

| | R₁ | R₂ | R₃ |
|---|---|---|---|
| Menogaril | H | OCH₃ | H |
| Nogalamycin | O-sugar | H | COOCH₃ |

sugar:

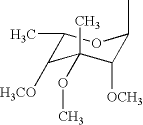

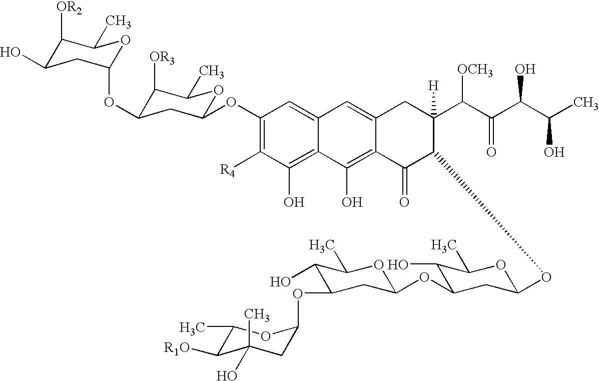

| | R₁ | R₂ | R₃ | R₄ |
|---|---|---|---|---|
| Olivomycin A | COCH(CH₃)₂ | CH₃ | COCH₃ | H |
| Chromomycin A₃ | COCH₃ | CH₃ | COCH₃ | CH₃ |
| Plicamycin | H | H | H | CH₃ |

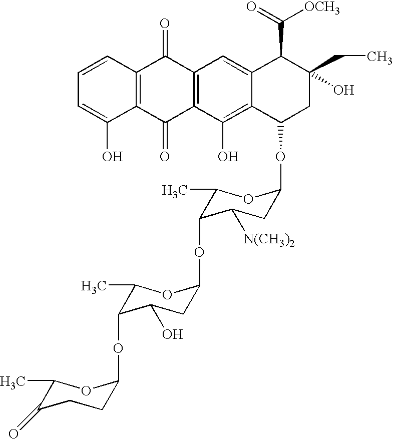

Aclacinomycin A

These compounds are thought to function as cell cycle inhibitors by being topoisomerase inhibitors and/or by DNA cleaving agents. They have been shown useful in the treatment of proliferative disorders, including small cell lung; breast; endometrial; head and neck; retinoblastoma; liver; bile duct; islet cell; and bladder cancers; and soft tissue sarcoma.

In another aspect, the cell cycle inhibitor is a platinum compound. In general, suitable platinum complexes may be of Pt(II) or Pt(IV) and have this basic structure: 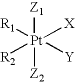
wherein X and Y are anionic leaving groups such as sulfate, phosphate, carboxylate, and halogen; R₁ and R₂ are alkyl, amine, amino alkyl any may be further substituted, and are basically inert or bridging groups. For Pt(II) complexes Z₁ and Z₂ are non-existent. For Pt(IV) Z₁ and Z₂ may be anionic groups such as halogen, hydroxy, carboxylate, ester, sulfate or phosphate. See, e.g., U.S. Pat. Nos. 4,588,831 and 4,250,189.

Suitable platinum complexes may contain multiple Pt atoms. See, e.g., U.S. Pat. Nos. 5,409,915 and 5,380,897. For example bisplatinum and triplatinum complexes of the type:

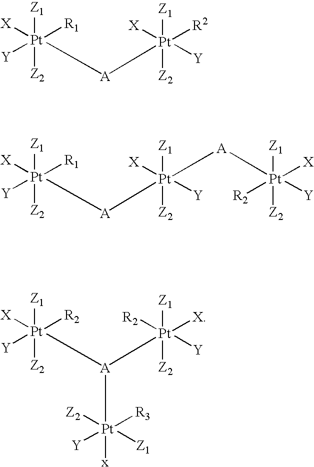

Exemplary platinum compounds are cisplatin, carboplatin, oxaliplatin, and miboplatin having the structures:

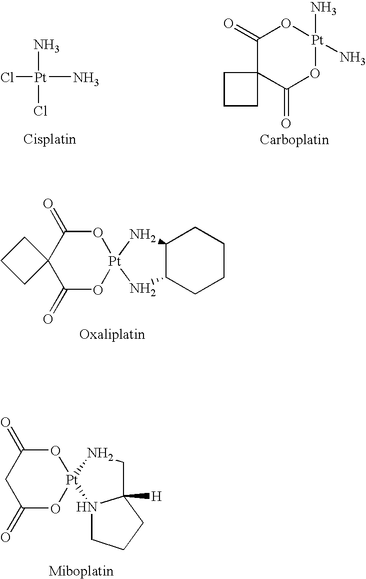

These compounds are thought to function as cell cycle inhibitors by binding to DNA, i.e., acting as alkylating agents of DNA. These compounds have been shown useful in the treatment of cell proliferative disorders, including, e.g., NSC lung; small cell lung; breast; cervical; brain; head and neck; esophageal; retinoblastom; liver; bile duct; bladder; penile; and vulvar cancers; and soft tissue sarcoma.

In another aspect, the cell cycle inhibitor is a nitrosourea. Nitrosourease have the following general structure (C5), where typical R groups are shown below.

| | |
|---|---|
| | (C5) |
| 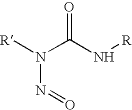 | |

R Group:

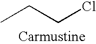

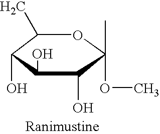

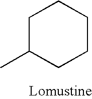

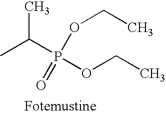

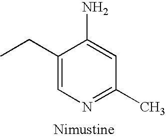

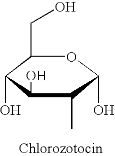

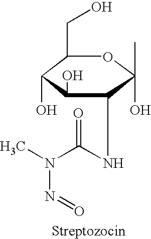

Other suitable R groups include cyclic alkanes, alkanes, halogen substituted groups, sugars, aryl and heteroaryl groups, phosphonyl and sulfonyl groups. As disclosed in U.S. Pat. No. 4,367,239, R may suitably be CH₂—C(X)(Y)(Z), wherein X and Y may be the same or different members of the following groups: phenyl, cyclyhexyl, or a phenyl or cyclohexyl group substituted with groups such as halogen, lower alkyl (C₁₋₄), trifluore methyl, cyano, phenyl, cyclohexyl, lower alkyloxy (C₁₋₄). Z has the following structure: -alkylene-N—R₁R₂, where R₁ and R₂ may be the same or different members of the following group: lower alkyl (C₁₋₄) and benzyl, or together R₁ and R₂ may form a saturated 5 or 6 membered heterocyclic such as pyrrolidine, piperidine, morfoline, thiomorfoline, N-lower alkyl piperazine, where the heterocyclic may be optionally substituted with lower alkyl groups.

As disclosed in U.S. Pat. No. 6,096,923, R and R′ of formula (C5) may be the same or different, where each may be a substituted or unsubstituted hydrocarbon having 1-10 carbons. Substitutions may include hydrocarbyl, halo, ester, amide, carboxylic acid, ether, thioether and alcohol groups. As disclosed in U.S. Pat. No. 4,472,379, R of formula (C5) may be an amide bond and a pyranose structure (e.g., methyl 2′-(N-(N-(2-chloroethyl)-N-nitroso-carbamoyl)-glycyl)amino-2′-deoxy-α-D-glucopyranoside). As disclosed in U.S. Pat. No. 4,150,146, R of formula (C5) may be an alkyl group of 2 to 6 carbons and may be substituted with an ester, sulfonyl, or hydroxyl group. It may also be substituted with a carboxylic acid or CONH₂ group.

Exemplary nitrosoureas are BCNU (carmustine), methyl-CCNU (semustine), CCNU (lomustine), ranimustine, nimustine, chlorozotocin, fotemustine, and streptozocin, having the structures:

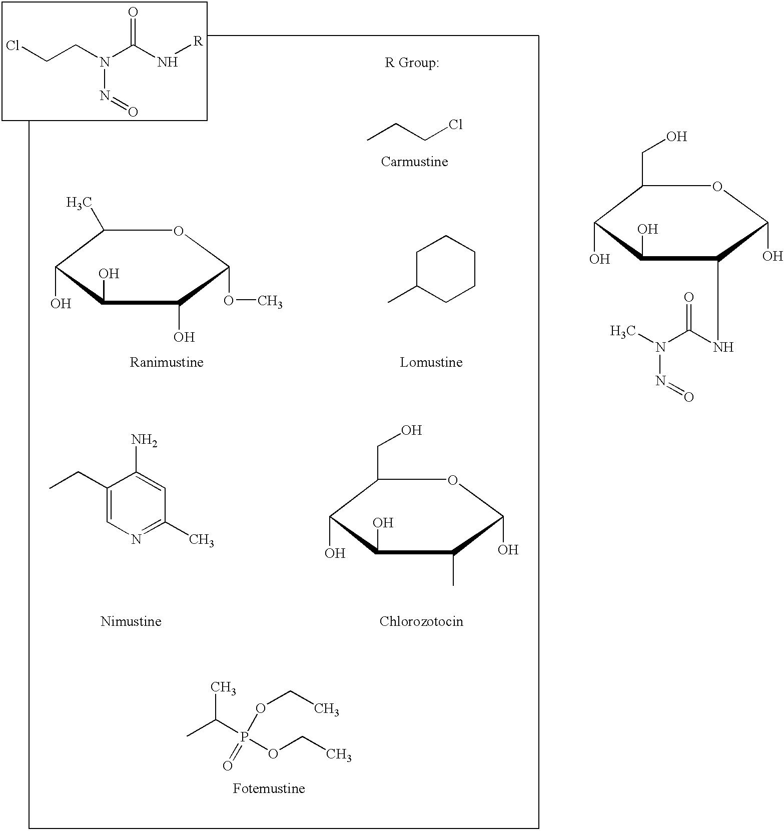

These nitrosourea compounds are thought to function as cell cycle inhibitors by binding to DNA, that is, by functioning as DNA alkylating agents. These cell cycle inhibitors have been shown useful in treating cell proliferative disorders such as, for example, islet cell; small cell lung; melanoma; and brain cancers.

In another aspect, the cell cycle inhibitor is a nitroimidazole, where exemplary nitroimidazoles are metronidazole, benznidazole, etanidazole, and misonidazole, having the structures:

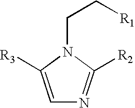

| | R₁ | R₂ | R₃ |
|---|---|---|---|
| Metronidazole | OH | CH₃ | NO₂ |
| Benznidazole | C(O)NHCH₂-benzyl | NO₂ | H |
| Etanidazole | CONHCH₂CH₂OH | NO₂ | H |

Suitable nitroimidazole compounds are disclosed in, e.g., U.S. Pat. Nos. 4,371,540 and 4,462,992.

In another aspect, the cell cycle inhibitor is a folic acid antagonist, such as methotrexate or derivatives or analogues thereof, including edatrexate, trimetrexate, raltitrexed, piritrexim, denopterin, tomudex, and pteropterin. Methotrexate analogues have the following general structure:

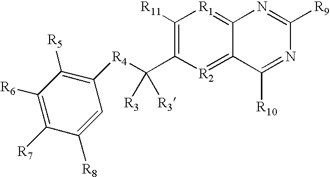

The identity of the R group may be selected from organic groups, particularly those groups set forth in U.S. Pat. Nos. 5,166,149 and 5,382,582. For example, R₁ may be N, R₂ may be N or C(CH₃), R₃ and R₃′ may H or alkyl, e.g., CH₃, R₄ may be a single bond or NR, where R is H or alkyl group. R_{5,6,8} may be H, OCH₃, or alternately they can be halogens or hydro groups. R₇ is a side chain of the general structure: 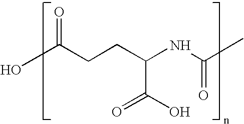
wherein n=1 for methotrexate, n=3 for pteropterin. The carboxyl groups in the side chain may be esterified or form a salt such as a Zn²⁺ salt. R₉ and R₁₀ can be NH₂ or may be alkyl substituted.

Exemplary folic acid antagonist compounds have the structures:

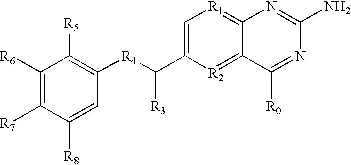

| | R₀ | R₁ | R₂ | R₃ | R₄ | R₅ | R₆ | R₇ | R₈ |
|---|---|---|---|---|---|---|---|---|---|
| Methotrexate | NH₂ | N | N | H | N(CH₃) | H | H | A(n = 1) | H |
| Edatrexate | NH₂ | N | N | H | N(CH₂CH₃) | H | H | A(n = 1) | H |
| Trimetrexate | NH₂ | N | C(CH₃) | H | NH | H | OCH₃ | OCH₃ | OCH₃ |
| Pteropterin | NH₂ | N | N | H | N(CH₃) | H | H | A(n = 3) | H |
| Denopterin | OH | N | N | CH₃ | N(CH₃) | H | H | A(n = 1) | H |
| Piritrexim | NH₂ | N | C(CH₃)H | single | OCH₃ | H | H | OCH₃ | H |
| | | | | bond | | | | | |

A:

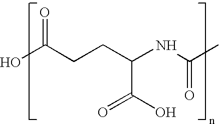

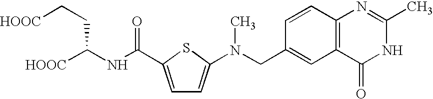

Tomudex

These compounds are thought to function as cell cycle inhibitors by serving as antimetabolites of folic acid. They have been shown useful in the treatment of cell proliferative disorders including, for example, soft tissue sarcoma, small cell lung, breast, brain, head and neck, bladder, and penile cancers.

In another aspect, the cell cycle inhibitor is a cytidine analogue, such as cytarabine or derivatives or analogues thereof, including enocitabine, FMdC ((E(-2′-deoxy-2′-(fluoromethylene)cytidine), gemcitabine, 5-azacitidine, ancitabine, and 6-azauridine. Exemplary compounds have the structures:

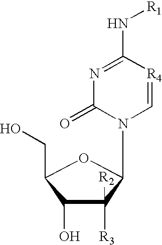

| | R₁ | R₂ | R₃ | R₄ |
|---|---|---|---|---|
| Cytarabine | H | OH | H | CH |
| Enocitabine | C(O)(CH₂)₂₀CH₃ | OH | H | CH |
| Gemcitabine | H | F | F | CH |
| Azacitidine | H | H | OH | N |
| FMdC | H | CH₂F | H | CH |

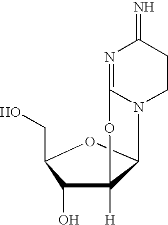

Ancitabine

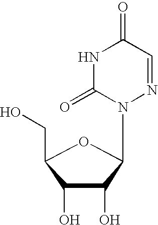

6-Azauridine

These compounds are thought to function as cell cycle inhibitors as acting as antimetabolites of pyrimidine. These compounds have been shown useful in the treatment of cell proliferative disorders including, for example, pancreatic, breast, cervical, NSC lung, and bile duct cancers.

In another aspect, the cell cycle inhibitor is a pyrimidine analogue. In one aspect, the pyrimidine analogues have the general structure: 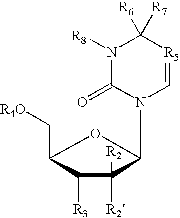
wherein positions 2′, 3′ and 5′ on the sugar ring (R₂, R₃ and R₄, respectively) can be H, hydroxyl, phosphoryl (see, e.g., U.S. Pat. No. 4,086,417) or ester (see, e.g., U.S. Pat. No. 3,894,000). Esters can be of alkyl, cycloalkyl, aryl or heterocyclo/aryl types. The 2′ carbon can be hydroxylated at either R₂ or R₂′, the other group is H. Alternately, the 2′ carbon can be substituted with halogens e.g., fluoro or difluoro cytidines such as Gemcytabine. Alternately, the sugar can be substituted for another heterocyclic group such as a furyl group or for an alkane, an alkyl ether or an amide linked alkane such as C(O)NH(CH₂)₅CH₃. The 2 amine can be substituted with an aliphatic acyl (R₁) linked with an amide (see, e.g., U.S. Pat. No. 3,991,045) or urethane (see, e.g., U.S. Pat. No. 3,894,000) bond. It can also be further substituted to form a quaternary ammonium salt. R₅ in the pyrimidine ring may be N or CR, where R is H, halogen containing groups, or alkyl (see, e.g., U.S. Pat. No. 4,086,417). R₆ and R₇ can together can form an oxo group or R₆═—NH—R, and R₇═H. R₈ is H or R₇ and R₈ together can form a double bond or R₈ can be X, where X is:

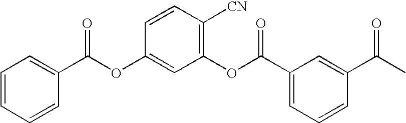

Specific pyrimidine analogues are disclosed in U.S. Pat. No. 3,894,000 (see, e.g., 2′-O-palmityl-ara-cytidine, 3′-O-benzoyl-ara-cytidine, and more than 10 other examples); U.S. Pat. No. 3,991,045 (see, e.g., N4-acyl-1-β-D-arabinofuranosylcytosine, and numerous acyl groups derivatives as listed therein, such as palmitoyl.

In another aspect, the cell cycle inhibitor is a fluoropyrimidine analogue, such as 5-fluorouracil, or an analogue or derivative thereof, including carmofur, doxifluridine, emitefur, tegafur, and floxuridine. Exemplary compounds have the structures:

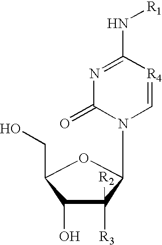

| | R₁ | R₂ |
|---|---|---|
| 5-Fluorouracil | H | H |
| Carmofur | C(O)NH(CH₂)₅CH₃ | H |
| Doxifluridine | A₁ | H |
| Floxuridine | A₂ | H |
| Emitefur | CH₂OCH₂CH₃ | B |
| Tegafur | | H |

A₁

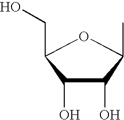

A₂

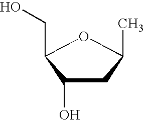

B

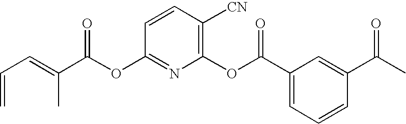

C

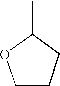

Other suitable fluoropyrimidine analogues include 5-FudR (5-fluoro-deoxyuridine), or an analogue or derivative thereof, including 5-iododeoxyuridine (5-ludR), 5-bromodeoxyuridine (5-BudR), fluorouridine triphosphate (5-FUTP), and fluorodeoxyuridine monophosphate (5-dFUMP). Exemplary compounds have the structures:

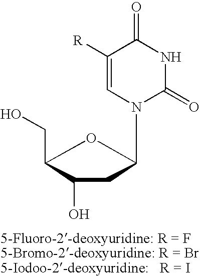

These compounds are thought to function as cell cycle inhibitors by serving as antimetabolites of pyrimidine. These compounds have been shown useful in the treatment of cell proliferative disorders such as breast, cervical, non-melanoma skin, head and neck, esophageal, bile duct, pancreatic, islet cell, penile, and vulvar cancers.

In another aspect, the cell cycle inhibitor is a purine analogue. Purine analogues have the following general structure. 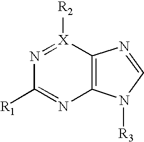
wherein X is typically carbon; R₁ is H, halogen, amine or a substituted phenyl; R₂ is H, a primary, secondary or tertiary amine, a sulfur containing group, typically —SH, an alkane, a cyclic alkane, a heterocyclic or a sugar; R₃ is H, a sugar (typically a furanose or pyranose structure), a substituted sugar or a cyclic or heterocyclic alkane or aryl group. See, e.g., U.S. Pat. No. 5,602,140 for compounds of this type.

In the case of pentostatin, X—R2 is —CH₂CH(OH)—. In this case a second carbon atom is inserted in the ring between X and the adjacent nitrogen atom. The X—N double bond becomes a single bond.

U.S. Pat. No. 5,446,139 describes suitable purine analogues of the type shown in the formula. 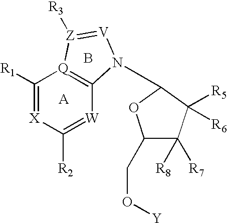
wherein N signifies nitrogen and V, W, X, Z can be either carbon or nitrogen with the following provisos. Ring A may have 0 to 3 nitrogen atoms in its structure. If two nitrogens are present in ring A, one must be in the W position. If only one is present, it must not be in the Q position. V and Q must not be simultaneously nitrogen. Z and Q must not be simultaneously nitrogen. If Z is nitrogen, R₃ is not present. Furthermore, R₁₋₃ are independently one of H, halogen, C₁₋₇ alkyl, C₁₋₇ alkenyl, hydroxyl, mercapto, C₁₋₇ alkylthio, C₁₋₇ alkoxy, C₂₋₇ alkenyloxy, aryloxy, nitro, primary, secondary or tertiary amine containing group. R₅₋₈ are H or up to two of the positions may contain independently one of OH, halogen, cyano, azido, substituted amino, R₅ and R₇ can together form a double bond. Y is H, a C₁₋₇ alkylcarbonyl, or a mono- di or tri phosphate.

Exemplary suitable purine analogues include 6-mercaptopurine, thiguanosine, thiamiprine, cladribine, fludaribine, tubercidin, puromycin, pentoxyfilline; where these compounds may optionally be phosphorylated. Exemplary compounds have the structures:

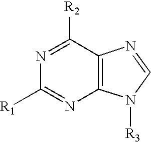

| | R₁ | R₂ | R₃ |
|---|---|---|---|
| 6-Mercaptopurine | H | SH | H |
| Thioguanosine | NH₂ | SH | B₁ |
| Thiamiprine | NH₂ | A | H |
| Cladribine | Cl | NH₂ | B₂ |
| Fludarabine | F | NH₂ | B₃ |
| Puromycin | H | N(CH₃)₂ | B₄ |
| Tubercidin | H | NH₂ | B₁ |

A:

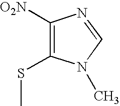

B₁:

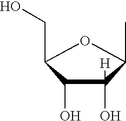

B₂:

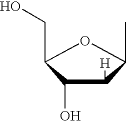

B₃:

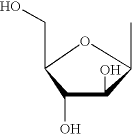

B₄:

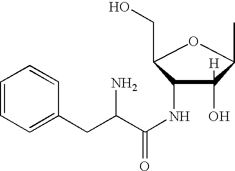

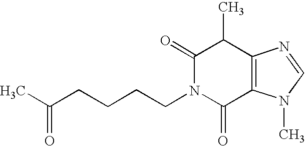

Pentoxyfilline

These compounds are thought to function as cell cycle inhibitors by serving as antimetabolites of purine.

In another aspect, the cell cycle inhibitor is a nitrogen mustard. Many suitable nitrogen mustards are known and are suitably used as a cell cycle inhibitor in the present invention. Suitable nitrogen mustards are also known as cyclophosphamides.

A preferred nitrogen mustard has the general structure: 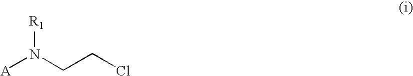
where A is: 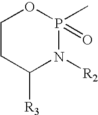
or —CH₃ or other alkane, or chloronated alkane, typically CH₂CH(CH₃)Cl, or a polycyclic group such as B, or a substituted phenyl such as C or a heterocyclic group such as D.

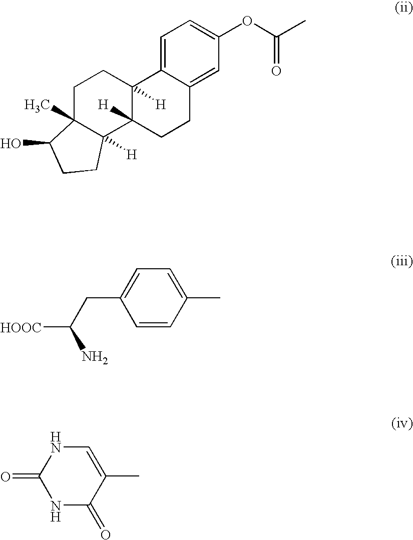

Examples of suitable nitrogen mustards are disclosed in U.S. Pat. No. 3,808,297, wherein A is: 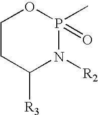
R₁₋₂ are H or CH₂CH₂Cl; R₃ is H or oxygen-containing groups such as hydroperoxy; and R₄ can be alkyl, aryl, heterocyclic.

The cyclic moiety need not be intact. See, e.g., U.S. Pat. Nos. 5 5,472,956, 4,908,356, 4,841,085 that describe the following type of structure: 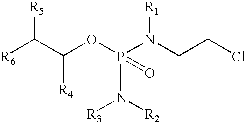
wherein R, is H or CH₂CH₂Cl, and R₂₋₆ are various substituent groups.

Exemplary nitrogen mustards include methylchloroethamine, and analogues or derivatives thereof, including methylchloroethamine oxide hydrohchloride, novembichin, and mannomustine (a halogenated sugar). Exemplary compounds have the structures:

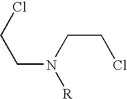

| | R |
|---|---|
| Mechlorethanime | CH₃ |
| Novembichin | CH₂CH(CH₃)Cl |

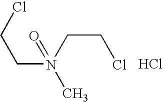

Mechlorethanime Oxide HCl

The nitrogen mustard may be cyclophosphamide, ifosfamide, perfosfamide, or torofosfamide, where these compounds have the structures:

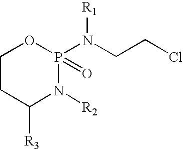

| | R₁ | R₂ | R₃ |
|---|---|---|---|
| Cyclophosphamide | H | CH₂CH₂Cl | H |
| Ifosfamide | CH₂CH₂Cl | H | H |
| Perfosfamide | CH₂CH₂Cl | H | OOH |
| Torofosfamide | CH₂CH₂Cl | CH₂CH₂Cl | H |

The nitrogen mustard may be estramustine, or an analogue or derivative thereof, including phenesterine, prednimustine, and estramustine PO₄. Thus, suitable nitrogen mustard type cell cycle inhibitors of the present invention have the structures:

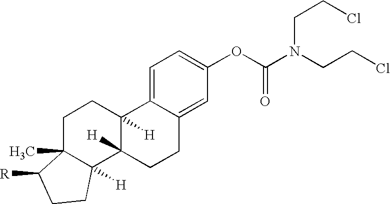

| | R |
|---|---|
| Estramustine | OH |
| Phenesterine | C(CH₃)(CH₂)₃CH(CH₃)₂ |

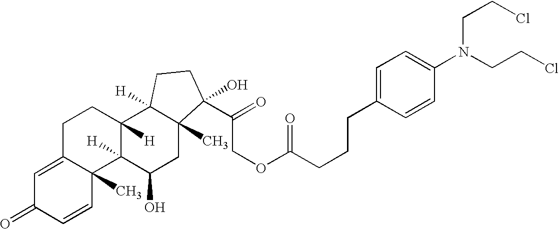

Prednimustine

The nitrogen mustard may be chlorambucil, or an analogue or derivative thereof, including melphalan and chlormaphazine. Thus, suitable nitrogen mustard type cell cycle inhibitors of the present invention have the structures:

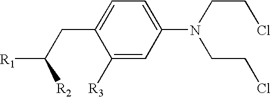

| | R₁ | R₂ | R₃ |
|---|---|---|---|
| Chlorambucil | CH₂COOH | H | H |
| Melphalan | COOH | NH₂ | H |

| | | |
|---|---|---|
| Chlornaphazine | H | together forms a |
| | | benzene ring |

The nitrogen mustard may be uracil mustard, which has the structure:

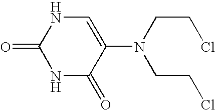

The nitrogen mustards are thought to function as cell cycle inhibitors by serving as alkylating agents for DNA. Nitrogen mustards have been shown useful in the treatment of cell proliferative disorders including, for example, small cell lung, breast, cervical, head and neck, prostate, retinoblastoma, and soft tissue sarcoma.

The cell cycle inhibitor of the present invention may be a hydroxyurea. Hydroxyureas have the following general structure:

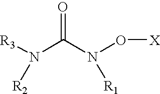

Suitable hydroxyureas are disclosed in, for example, U.S. Pat. No. 6,080,874, wherein R₁ is: 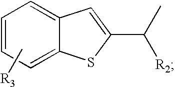
and R₂ is an alkyl group having 1-4 carbons and R₃ is one of H, acyl, methyl, ethyl, and mixtures thereof, such as a methylether.

Other suitable hydroxyureas are disclosed in, e.g., U.S. Pat. No. 5,665,768, wherein R₁ is a cycloalkenyl group, for example N-(3-(5-(4-fluorophenylthio)-furyl)-2-cyclopenten-1-yl)N-hydroxyurea; R₂ is H or an alkyl group having 1 to 4 carbons and R₃ is H; X is H or a cation.

Other suitable hydroxyureas are disclosed in, e.g., U.S. Pat. No. 4,299,778, wherein R₁ is a phenyl group substituted with on or more fluorine atoms; R₂ is a cyclopropyl group; and R₃ and X is H.

Other suitable hydroxyureas are disclosed in, e.g., U.S. Pat. No. 5,066,658, wherein R₂ and R₃ together with the adjacent nitrogen form: 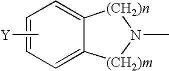
wherein m is 1 or 2, n is 0-2 and Y is an alkyl group.

In one aspect, the hydroxy urea has the structure:

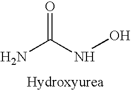

Hydroxyureas are thought to function as cell cycle inhibitors by serving to inhibit DNA synthesis.

In another aspect, the cell cycle inhibitor is a mytomicin, such as mitomycin C, or an analogue or derivative thereof, such as porphyromycin. Exemplary compounds have the structures:

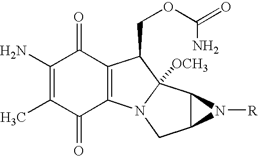

| | R |
|---|---|
| Mitomycin C | H |
| Porphyromycin | CH₃ |
| (N-methyl Mitomycin C) | |

These compounds are thought to function as cell cycle inhibitors by serving as DNA alkylating agents. Mitomycins have been shown useful in the treatment of cell proliferative disorders such as, for example, esophageal, liver, bladder, and breast cancers.

In another aspect, the cell cycle inhibitor is an alkyl sulfonate, such as busulfan, or an analogue or derivative thereof, such as treosulfan, improsulfan, piposulfan, and pipobroman. Exemplary compounds have the structures:

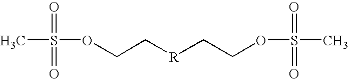

| | R |
|---|---|
| Busulfan | single bond |
| Improsulfan | —CH₂—NH—CH₂— |
| Piposulfan | 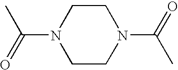 |

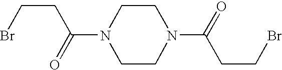

Pipobroman

These compounds are thought to function as cell cycle inhibitors by serving as DNA alkylating agents.

In another aspect, the cell cycle inhibitor is a benzamide. In yet another aspect, the cell cycle inhibitor is a nicotinamide. These compounds have the basic structure: 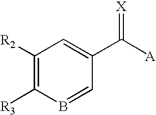
wherein X is either O or S; A is commonly NH₂ or it can be OH or an alkoxy group; B is N or C—R₄, where R₄ is H or an ether-linked hydroxylated alkane such as OCH₂CH₂OH, the alkane may be linear or branched and may contain one or more hydroxyl groups. Alternately, B may be N—R₅ in which case the double bond in the ring involving B is a single bond. R₅ may be H, and alkyl or an aryl group (see, e.g., U.S. Pat. No. 4,258,052); R₂ is H, OR₆, SR₆ or NHR₆, where R₆ is an alkyl group; and R₃ is H, a lower alkyl, an ether linked lower alkyl such as —O-Me or —O-ethyl (see, e.g., U.S. Pat. No. 5,215,738).

Suitable benzamide compounds have the structures: 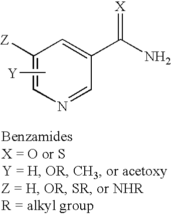
where additional compounds are disclosed in U.S. Pat. No. 5,215,738, (listing some 32 compounds).

Suitable nicotinamide compounds have the structures:

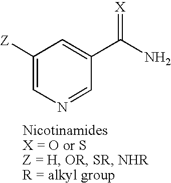

where additional compounds are disclosed in U.S. Pat. No. 5,215,738,

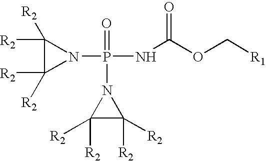

| | R₁ | R₂ |
|---|---|---|
| Benzodepa | phenyl | H |
| Meturedepa | CH₃ | CH₃ |
| Uredepa | CH₃ | H |

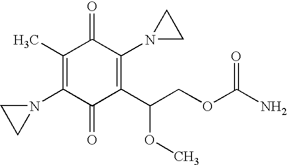

Carboquone

In another aspect, the cell cycle inhibitor is a halogenated sugar, such as mitolactol, or an analogue or derivative thereof, including mitobronitol and mannomustine. Examplary compounds have the structures:

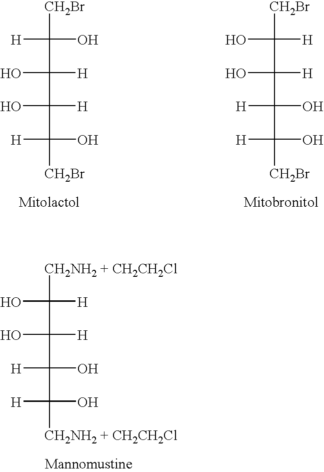

In another aspect, the cell cycle inhibitor is a diazo compound, such as azaserine, or an analogue or derivative thereof, including 6-diazo-5-oxo-L-norleucine and 5-diazouracil (also a pyrimidine analog). Examplary compounds have the structures:

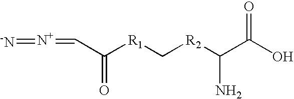

| | R₁ | R₂ |
|---|---|---|
| Azaserine | O | single bond |
| 6-diazo-5-oxo- | single bond | CH₂ |
| L-norleucine | | |

Other compounds that may serve as cell cycle inhibitors according to the present invention are pazelliptine; wortmannin; metoclopramide; RSU; buthionine sulfoxime; tumeric; curcumin; AG337, a thymidylate synthase inhibitor; levamisole; lentinan, a polysaccharide; razoxane, an EDTA analogue; indomethacin; chlorpromazine; α and β interferon; MnBOPP; gadolinium texaphyrin; 4-amino-1,8-naphthalimide; staurosporine derivative of CGP; and SR-2508.

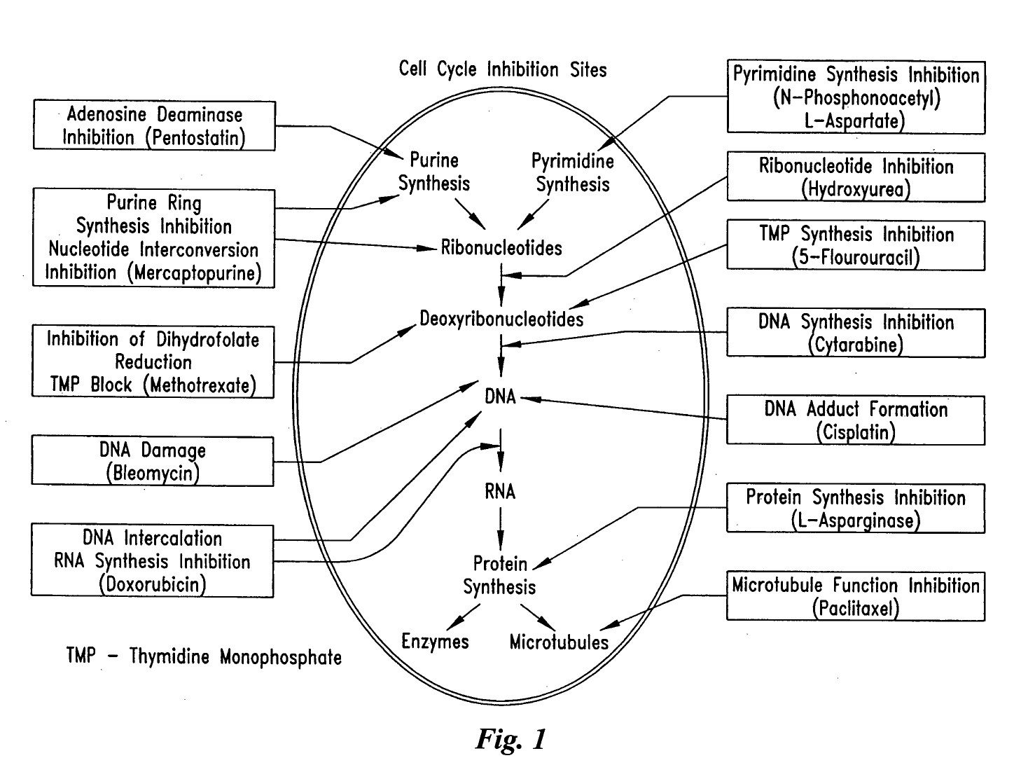

Thus, in one aspect, the cell cycle inhibitor is a DNA alylating agent. In another aspect, the cell cycle inhibitor is an anti-microtubule agent. In another aspect, the cell cycle inhibitor is a topoisomerase inhibitor. In another aspect, the cell cycle inhibitor is a DNA cleaving agent. In another aspect, the cell cycle inhibitor is an antimetabolite. In another aspect, the cell cycle inhibitor functions by inhibiting adenosine deaminase (e.g., as a purine analogue). In another aspect, the cell cycle inhibitor functions by inhibiting purine ring synthesis and/or as a nucleotide interconversion inhibitor (e.g., as a purine analogue such as mercaptopurine). In another aspect, the cell cycle inhibitor functions by inhibiting dihydrofolate reduction and/or as a thymidine monophosphate block (e.g., methotrexate). In another aspect, the cell cycle inhibitor functions by causing DNA damage (e.g., bleomycin). In another aspect, the cell cycle inhibitor functions as a DNA intercalation agent and/or RNA synthesis inhibition (e.g., doxorubicin, aclarubicin, or detorubicin (acetic acid, diethoxy-, 2-(4-((3-amino-2,3,6-trideoxy-alpha-L-lyxo-hexopyranosyl)oxy)-1,2,3,4,6,11-hexahydro-2,5,12-trihydroxy-7-methoxy-6,11-dioxo-2-naphthacenyl)-2-oxoethyl ester, (2S-cis)-)). In another aspect, the cell cycle inhibitor functions by inhibiting pyrimidine synthesis (e.g., N-phosphonoacetyl-L-aspartate). In another aspect, the cell cycle inhibitor functions by inhibiting ribonucleotides (e.g., hydroxyurea). In another aspect, the cell cycle inhibitor functions by inhibiting thymidine monophosphate (e.g., 5-fluorouracil). In another aspect, the cell cycle inhibitor functions by inhibiting DNA synthesis (e.g., cytarabine). In another aspect, the cell cycle inhibitor functions by causing DNA adduct formation (e.g., platinum compounds). In another aspect, the cell cycle inhibitor functions by inhibiting protein synthesis (e.g., L-asparginase). In another aspect, the cell cycle inhibitor functions by inhibiting microtubule function (e.g., taxanes). In another aspect, the cell cycle inhibitor acts at one or more of the steps in the biological pathway shown in FIG. 1.

Additional cell cycle inhibitor s useful in the present invention, as well as a discussion of the mechanisms of action, may be found in Hardman J. G., Limbird L. E. Molinoff R. B., Ruddon R W., Gilman A. G. editors, Chemotherapy of Neoplastic Diseases in Goodman and Gilman's The Pharmacological Basis of Therapeutics Ninth Edition, McGraw-Hill Health Professions Division, New York, 1996, pages 1225-1287. See also U.S. Pat. Nos. 3,387,001; 3,808,297; 3,894,000; 3,991,045; 4,012,390; 4,057,548; 4,086,417; 4,144,237; 4,150,146; 4,210,584; 4,215,062; 4,250,189; 4,258,052; 4,259,242; 4,296,105; 4,299,778; 4,367,239; 4,374,414; 4,375,432; 4,472,379; 4,588,831; 4,639,456; 4,767,855; 4,828,831; 4,841,045; 4,841,085; 4,908,356; 4,923,876; 5,030,620; 5,034,320; 5,047,528; 5,066,658; 5,166,149; 5,190,929; 5,215,738; 5,292,731; 5,380,897; 5,382,582; 5,409,915; 5,440,056; 5,446,139; 5,472,956; 5,527,905; 5,552,156; 5,594,158; 5,602,140; 5,665,768; 5,843,903; 6,080,874; 6,096,923; and RE030561.

In another embodiment, the cell-cycle inhibitor is camptothecin, mitoxantrone, etoposide, 5-fluorouracil, doxorubicin, methotrexate, peloruside A, mitomycin C, or a CDK-2 inhibitor or an analogue or derivative of any member of the class of listed compounds.

In another embodiment, the cell-cycle inhibitor is HTI-286, plicamycin; or mithramycin, or an analogue or derivative thereof.

Other examples of cell cycle inhibitors also include, e.g., 7-hexanoyltaxol (QP-2), cytochalasin A, lantrunculin D, actinomycin-D, Ro-31-7453 (3-(6-nitro-1-methyl-3-indolyl)-4-(1-methyl-3-indolyl)pyrrole-2,5-dione), PNU-151807, brostallicin, C2-ceramide, cytarabine ocfosfate(2(1H)-pyrimidinone, 4-amino-1-(5-O-(hydroxy(octadecyloxy)phosphinyl)-β-D-arabinofuranosyl)-, monosodium salt), paclitaxel (5β,20-epoxy-1,2 alpha,4,7β,10β,13 alpha-hexahydroxytax-11-en-9-one-4,10-diacetate-2-benzoate-13-(alpha-phenylhippurate)), doxorubicin(5,12-naphthacenedione, 10-((3-amino-2,3,6-trideoxy-alpha-L-lyxo-hexopyranosyl)oxy)-7,8,9,10-tetrahydro-6,8,11-trihydroxy-8-(hydroxyacetyl)-1-methoxy-, (8S)-cis-), daunorubicin (5,12-naphthacenedione, 8-acetyl-10-((3-amino-2,3,6-trideoxy-alpha-L-lyxo-hexopyranosyl)oxy)-7,8,9,10-tetrahydro-6,8,11-trihydroxy-1-methoxy-, (8S-cis)-), gemcitabine hydrochloride (cytidine, 2′-deoxy-2′,2′-difluoro-,monohydrochloride), nitacrine (1,3-propanediamine, N,N-dimethyl-N′-(1-nitro-9-acridinyl)-), carboplatin (platinum, diammine(1,1-cyclobutanedicarboxylato(2-))-, (SP-4-2)-), altretamine (1,3,5-triazine-2,4,6-triamine, N,N,N′,N′,N″,N″-hexamethyl-), teniposide (furo(3′,4′:6,7)naphtho(2,3-d)-1,3-dioxol-6(5aH)-one, 5,8,8a ,9-tetrahydro-5-(4-hydroxy-3,5-dimethoxyphenyl)-9-((4,6-O-(2-thienylmethylene)-β-D-glucopyranosyl)oxy)-, (5R-(5alpha,5aβ,8aAlpha,9β(R*)))-), eptaplatin (platinum, ((4R,5R)-2-(1-methylethyl)-1,3-dioxolane-4,5-dimethanamine-kappa N4,kappa N5)(propanedioato(2-)-kappa O1, kappa O3)-, (SP-4-2)-), amrubicin hydrochloride (5,12-naphthacenedione, 9-acetyl-9-amino-7-((2-deoxy-β-D-erythro-pentopyranosyl)oxy)-7,8,9,10-tetrahydro-6,11-dihydroxy-, hydrochloride, (7S-cis)-), ifosfamide (2H-1,3,2-oxazaphosphorin-2-amine, N,3-bis(2-chloroethyl)tetrahydro-,2-oxide), cladribine (adenosine, 2-chloro-2′-deoxy-), mitobronitol (D-mannitol, 1,6-dibromo-1,6-dideoxy-), fludaribine phosphate (9H-purin-6-amine, 2-fluoro-9-(5-O-phosphono-β-D-arabinofuranosyl)-), enocitabine (docosanamide, N-(1-β-D-arabinofuranosyl-1,2-dihydro-2-oxo-4-pyrimidinyl)-), vindesine (vincaleukoblastine, 3-(aminocarbonyl)-O4-deacetyl-3-de(methoxycarbonyl)-), idarubicin (5,12-naphthacenedione, 9-acetyl-7-((3-amino-2,3,6-trideoxy-alpha-L-lyxo-hexopyranosyl)oxy)-7,8,9,10-tetrahydro-6,9,11-trihydroxy-, (7S-cis)-), zinostatin (neocarzinostatin), vincristine (vincaleukoblastine, 22-oxo-), tegafur (2,4(1H,3H)-pyrimidinedione, 5-fluoro-1-(tetrahydro-2-furanyl)-), razoxane(2,6-piperazinedione, 4,4′-(1-methyl-1,2-ethanediyl)bis-), methotrexate (L-glutamic acid, N-(4-(((2,4-diamino-6-pteridinyl)methyl)methylamino)benzoyl)-), raltitrexed (L-glutamic acid, N-((5-(((1,4-dihydro-2-methyl-4-oxo-6-quinazolinyl)methyl)methylamino)-2-thienyl)carbonyl)-), oxaliplatin (platinum, (1,2-cyclohexanediamine-N,N′)(ethanedioato(2-)-O,O′)—, (SP-4-2-(1R-trans))-), doxifluridine (uridine, 5′-deoxy-5-fluoro-), mitolactol (galactitol, 1,6-dibromo-1,6-dideoxy-), piraubicin (5,12-naphthacenedione, 10-((3-amino-2,3,6-trideoxy-4-O-(tetrahydro-2H-pyran-2-yl)-alpha-L-lyxo-hexopyranosyl)oxy)-7,8,9,1 0-tetrahydro-6,8,11-trihydroxy-8-(hydroxyacetyl)-1-methoxy-, (8S-(8 alpha, 10 alpha(S*)))—), docetaxel ((2R,3S)—N-carboxy-3-phenylisoserine, N-tert-butyl ester, 13-ester with 5β,20-epoxy-1,2alpha,4,7β,10β,13 alpha-hexahydroxytax-11-en-9-one 4-acetate 2-benzoate-), capecitabine (cytidine, 5-deoxy-5-fluoro-N-((pentyloxy)carbonyl)-), cytarabine (2(1H)-pyrimidone, 4-amino-1-β-D-arabino furanosyl-), valrubicin (pentanoic acid, 2-(1,2,3,4,6,11-hexahydro-2,5,12-trihydroxy-7-methoxy-6,11-dioxo-4-((2,3,6-trideoxy-3-((trifluoroacetyl)amino)-alpha-L-lyxo-hexopyranosyl)oxy)-2-naphthacenyl)-2-oxoethyl ester (2S-cis)-), trofosfamide (3-2-(chloroethyl)-2-(bis(2-chloroethyl)amino)tetrahydro-2H-1,3,2-oxazaphosphorin 2-oxide), prednimustine (pregna-1,4-diene-3,20-dione, 21-(4-(4-(bis(2-chloroethyl)amino)phenyl)-1-oxobutoxy)-1,11 7-dihydroxy-, (11β)-), lomustine (Urea, N-(2-chloroethyl)-N′-cyclohexyl-N-nitroso-), epirubicin (5,12-naphthacenedione, 10-((3-amino-2,3,6-trideoxy-alpha-L-arabino-hexopyranosyl)oxy)-7,8,9,10-tetrahydro-6,8,11-trihydroxy-8-(hydroxyacetyl)-1-methoxy-, (8S-cis)-), or an analogue or derivative thereof).

5) Cyclin Dependent Protein Kinase Inhibitors

In another embodiment, the pharmacologically active fibrosis-inhibiting compound is a cyclin dependent protein kinase inhibitor (e.g., R-roscovitine, CYC-101, CYC-103, CYC-400, MX-7065, alvocidib (4H-1-Benzopyran-4-one, 2-(2-chlorophenyl)-5,7-dihydroxy-8-(3-hydroxy-1-methyl-4-piperidinyl)-, cis-(−)-), SU-9516, AG-12275, PD-0166285, CGP-79807, fascaplysin, GW-8510 (benzenesulfonamide, 4-(((Z)-(6,7-dihydro-7-oxo-8H-pyrrolo(2,3-g)benzothiazol-8-ylidene)methyl)amino)-N-(3-hydroxy-2,2-dimethylpropyl)-), GW-491619, Indirubin 3′ monoxime, GW8510, AZD-5438, ZK-CDK or an analogue or derivative thereof).

6) EGF (Epidermal Growth Factor) Receptor Kinase Inhibitors

In another embodiment, the pharmacologically active fibrosis-inhibiting compound is an EGF (epidermal growth factor) kinase inhibitor (e.g., erlotinib (4-quinazolinamine, N-(3-ethynylphenyl)-6,7-bis(2-methoxyethoxy)-,monohydrochloride), erbstatin, BIBX-1382, gefitinib (4-quinazolinamine, N-(3-chloro-4-fluorophenyl)-7-methoxy-6-(3-(4-morpholinyl)propoxy)), or an analogue or derivative thereof).

7) Elastase Inhibitors

In another embodiment, the pharmacologically active fibrosis-inhibiting compound is an elastase inhibitor (e.g., ONO-6818, sivelestat sodium hydrate (glycine, N-(2-(((4-(2,2-dimethyl-1-oxopropoxy)phenyl)sulfonyl)amino)benzoyl)-), erdosteine (acetic acid, ((2-oxo-2-((tetrahydro-2-oxo-3-thienyl)amino)ethyl)thio)-), MDL-100948A, MDL-104238 (N-(4-(4-morpholinylcarbonyl)benzoyl)-L-valyl-N′-(3,3,4,4,4-pentafluoro-1-(1-methylethyl)-2-oxobutyl)-L-2-azetamide), MDL-27324 (L-prolinamide, N-((5-(dimethylamino)-1-naphthalenyl)sulfonyl)-L-alanyl-L-alanyl-N-(3,3,3-trifluoro-1-(1-methylethyl)-2-oxopropyl)-, (S)—), SR-26831 (thieno(3,2-c)pyridinium, 5-((2-chlorophenyl)methyl )-2-(2,2-dimethyl-1-oxopropoxy)-4,5,6,7-tetrahydro-5-hydroxy-), Win-68794, Win-63110, SSR-69071 (2-(9(2-piperidinoethoxy)-4-oxo-4H-pyrido(1,2-a)pyrimidin-2-yloxymethyl)-4-(1-methylethyl)-6-methyoxy-1,2-benzisothiazol-3(2H)-one-1,1-dioxide), (N(Alpha)-(1-adamantylsulfonyl)N(epsilon)-succinyl-L-lysyl-L-prolyl-L-valinal), Ro-31-3537 (N alpha-(1-adamantanesulphonyl)-N-(4-carboxybenzoyl)-L-lysyl-alanyl-L-valinal), R-665, FCE-28204, ((6R,7R)-2-(benzoyloxy)-7-methoxy-3-methyl-4-pivaloyl-3-cephem 1,1-dioxide), 1,2-benzisothiazol-3(2H)-one, 2-(2,4-dinitrophenyl)-, 1,1-dioxide, L-658758 (L-proline, 1-((3-((acetyloxy)methyl)-7-methoxy-8-oxo-5-thia-1-azabicyclo(4.2.0)oct-2-en-2-yl)carbonyl)-, S,S-dioxide, (6R-cis)-), L-659286 (pyrrolidine, 1-((7-methoxy-8-oxo-3-(((1,2,5,6-tetrahydro-2-methyl-5,6-dioxo-1,2,4-triazin-3-yl)thio)methyl)-5-thia-1-azabicyclo(4.2.0)oct-2-en-2-yl)carbonyl)-, S,S-dioxide, (6R-cis)-), L-680833 (benzeneacetic acid, 4-((3,3-diethyl-1-(((1-(4-methylphenyl)butyl)amino)carbonyl)-4-oxo-2-azetidinyl)oxy)-, (S—(R*,S*))—), FK-706 (L-prolinamide, N-(4-(((carboxymethyl)amino)carbonyl)benzoyl)-L-valyl-N-(3,3,3-trifluoro-1-(1-methylethyl)-2-oxopropyl)-, monosodium salt), Roche R-665, or an analogue or derivative thereof).

8) Factor Xa Inhibitors

In another embodiment, the pharmacologically active fibrosis-inhibiting compound is a factor Xa inhibitor (e.g., CY-222, fondaparinux sodium (alpha-D-glucopyranoside, methyl O-2-deoxy-6-O-sulfo-2-(sulfoamino)-alpha-D-glucopyranosyl-(1-4)-O-β-D-glucopyranuronosyl-(1-4)-O-2-deoxy-3,6-di-O-sulfo-2-(sulfoamino)-alpha-D-glucopyranosyl-(1-4)-O-2-O-sulfo-alpha-L-idopyranuronosyl-(1-4)-2-deoxy-2-(sulfoamino)-, 6-(hydrogen sulfate)), danaparoid sodium, or an analogue or derivative thereof).

9) Farnesyltransferase Inhibitors

In another embodiment, the pharmacologically active fibrosis-inhibiting compound is a farnesyltransferase inhibitor (e.g., dichlorobenzoprim (2,4-diamino-5-(4-(3,4-dichlorobenzylamino)-3-nitrophenyl)-6-ethylpyrimidine), B-581, B-956 (N-(8(R)-amino-2(S)-benzyl-5(S)-isopropyl-9-sulfanyl-3(Z),6(E)-nonadienoyl)-L-methionine), OSI-754, perillyl alcohol (1-cyclohexene-1-methanol, 4-(1-methylethenyl)-, RPR-114334, lonafarnib (1-piperidinecarboxamide, 4-(2-(4-((11R)-3,10-dibromo-8-chloro-6,11-dihydro-5H-benzo(5,6)cyclohepta(1,2-b)pyridin-11-yl)-1-piperidinyl)-2-oxoethyl)-), Sch-48755, Sch-226374, (7,8-dichloro-5H-dibenzo(b,e)(1,4)diazepin-11-y1)-pyridin-3-ylmethylamine, J-104126, L-639749, L-731734 (pentanamide, 2-((2-((2-amino-3-mercaptopropyl)amino)-3-methylpentyl)amino)-3-methyl-N-(tetrahydro-2-oxo-3-furanyl)-, (3S-(3R*(2R*(2R*(S*),3S*),3R*)))—), L-744832 (butanoic acid, 2-((2-((2-((2-amino-3-mercaptopropyl)amino)-3-methylpentyl)oxy)-1-oxo-3-phenylpropyl)amino)-4-(methylsulfonyl)-, 1-methylethyl ester, (2S-(1(R*(R*)),2R*(S*),3R*))—), L-745631 (1-piperazinepropanethiol, β-amino-2-(2-methoxyethyl)-4-(1-naphthalenylcarbonyl)-, (βR,2S)—), N-acetyl-N-naphthylmethyl-2(S)-((1-(4-cyanobenzyl)-1H-imidazol-5-yl)acetyl)amino-3(S)-methylpentamine, (2alpha)-2-hydroxy-24,25-dihydroxylanost-8-en-3-one, BMS-316810, UCF-1-C (2,4-decadienamide, N-(5-hydroxy-5-(7-((2-hydroxy-5-oxo-1-cyclopenten-l-yl)amino-oxo-1,3,5-heptatrienyl)-2-oxo-7-oxabicyclo(4.1.0)hept-3-en-3-yl)-2,4,6-trimethyl-, (1S-(1alpha,3(2E,4E,6S*),5 alpha, 5(1E,3E,5E), 6 alpha))-), UCF-116-B, ARGLABIN (3H-oxireno(8,8a)azuleno(4,5-b)furan-8(4aH)-one, 5,6,6a,7,9a,9b-hexahydro-1,4a-dimethyl-7-methylene-, (3aR,4aS,6aS,9aS,9bR)-) from ARGLABIN—Paracure, Inc. (Virginia Beach, Va.), or an analogue or derivative thereof).

10) Fibrinogen Antagonists

In another embodiment, the pharmacologically active fibrosis-inhibiting compound is a fibrinogen antagonist (e.g., 2(S)-((p-toluenesulfonyl)amino)-3-(((5,6,7,8,-tetrahydro-4-oxo-5-(2-(piperidin-4-yl)ethyl)-4H-pyrazolo-(1,5-a)(1,4)diazepin-2-yl)carbonyl)-amino)propionic acid, streptokinase (kinase (enzyme-activating), strepto-), urokinase (kinase (enzyme-activating), uro-), plasminogen activator, pamiteplase, monteplase, heberkinase, anistreplase, alteplase, pro-urokinase, picotamide (1,3-benzenedicarboxamide, 4-methoxy-N,N′-bis(3-pyridinylmethyl)-), or an analogue or derivative thereof).

11) Guanylate Cyclase Stimulants

In another embodiment, the pharmacologically active fibrosis-inhibiting compound is a guanylate cyclase stimulant (e.g., isosorbide-5-mononitrate (D-glucitol, 1,4:3,6-dianhydro-, 5-nitrate), or an analogue or derivative thereof).

12) Heat Shock Protein 90 Antagonists

In another embodiment, the pharmacologically active fibrosis-inhibiting compound is a heat shock protein 90 antagonist (e.g., geldanamycin; NSC-33050 (17-allylaminogeldanamycin), rifabutin (rifamycin XIV, 1′,4-didehydro-1-deoxy-1,4-dihydro-5′-(2-methylpropyl)-1-oxo-), 17AAG, or an analogue or derivative thereof).

13) HMGCoA Reductase Inhibitors

In another embodiment, the pharmacologically active fibrosis-inhibiting compound is an HMGCoA reductase inhibitor (e.g., BCP-671, BB-476, fluvastatin (6-heptenoic acid, 7-(3-(4-fluorophenyl)-1-(1-methylethyl)-1H-indol-2-yl)-3,5-dihydroxy-, monosodium salt, (R*,S*-(E))-(±)-), dalvastatin (2H-pyran-2-one, 6-(2-(2-(2-(4-fluoro-3-methylphenyl)-4,4,6,6-tetramethyl-1-cyclohexen-1-yl)ethenyl)tetrahydro)-4-hydroxy-, (4alpha,6βE))-(±)-), glenvastatin (2H-pyran-2-one, 6-(2-(4-(4-fluorophenyl)-2-(1-methylethyl)-6-phenyl-3-pyridinyl)ethenyl)tetrahydro-4-hydroxy-, (4R-(4alpha,6β(E)))-), S-2468, N-(1-oxododecyl)-4Alpha,10-dimethyl-8-aza-trans-decal-3β-ol, atorvastatin calcium (1H-Pyrrole-1-heptanoic acid, 2-(4-fluorophenyl)-β,delta-dihydroxy-5-(1-methylethyl)-3-phenyl-4-((phenylamino)carbonyl)-, calcium salt (R—(R*,R*))—), CP-83101 (6,8-nonadienoic acid, 3,5-dihydroxy-9,9-diphenyl-, methyl ester, (R*,S*-(E))-(±)-), pravastatin (1-naphthaleneheptanoic acid, 1,2,6,7,8,8a-hexahydro-β,delta,6-trihydroxy-2-methyl-8-(2-methyl-1-oxobutoxy)-, monosodium salt, (1S-(1 alpha(βS*,deltaS*),2 alpha,6 alpha,8β(R*),8a alpha))-), U-20685, pitavastatin (6-heptenoic acid, 7-(2-cyclopropyl-4-(4-fluorophenyl)-3-quinolinyl)-3,5-dihydroxy-, calcium salt (2:1), (S—(R*,S*-(E)))-), N-((1-methylpropyl)carbonyl)-8-(2-(tetrahydro-4-hydroxy-6-oxo-2H-pyran-2-yl)ethyl)-perhydro-isoquinoline, dihydromevinolin (butanoic acid, 2-methyl-, 1,2,3,4,4a,7,8,8a-octahydro-3,7-dimethyl-8-(2-(tetrahydro-4-hydroxy-6-oxo-2H-pyran-2-yl)ethyl)-1-naphthalenyl ester(1 alpha(R*), 3 alpha, 4a alpha,7β,8β(2S*,4S*),8aβ))-), HBS-107, dihydromevinolin (butanoic acid, 2-methyl-, 1,2,3,4,4a ,7,8,8a-octahydro-3,7-dimethyl-8-(2-(tetrahydro-4-hydroxy-6-oxo-2H-pyran-2-yl)ethyl)-1-naphthalenyl ester(1 alpha(R*), 3 alpha,4a alpha,7β,8β(2S*,4S*),8aβ))-), L-669262 (butanoic acid, 2,2-dimethyl-, 1,2,6,7,8,8a-hexahydro-3,7-dimethyl-6-oxo-8-(2-(tetrahydro-4-hydroxy-6-oxo-2H-pyran-2-yl)ethyl)-1-naphthalenyl(1S-(1Alpha,7β,8β(2S*,4S*),8aβ))-), simvastatin (butanoic acid, 2,2-dimethyl-, 1,2,3,7,8,8a-hexahydro-3,7-dimethyl-8-(2-(tetrahydro-4-hydroxy-6-oxo-2H-pyran-2-yl)ethyl)-1-naphthalenyl ester, (1S-(1alpha, 3alpha,7β,8β(2S*,4S*),8aβ))-), rosuvastatin calcium (6-heptenoic acid, 7-(4-(4-fluorophenyl)-6-(1-methylethyl)-2-(methyl(methylsulfonyl)amino)-5-pyrimdinyl)-3,5-dihydroxy-calcium salt (2:1) (S—(R*, S*-(E)))), meglutol (2-hydroxy-2-methyl-1,3-propandicarboxylic acid), lovastatin (butanoic acid, 2-methyl-, 1,2,3,7,8,8a-hexahydro-3,7-dimethyl-8-(2-(tetrahydro-4-hydroxy-6-oxo-2H-pyran-2-yl)ethyl)-1-naphthalenyl ester, (1S-(1 alpha.(R*),3 alpha,7β,8β(2S*,4S*),8aβ))-), or an analogue or derivative thereof).

14) Hydroorotate Dehydrogenase Inhibitors

In another embodiment, the pharmacologically active fibrosis-inhibiting compound is a hydroorotate dehydrogenase inhibitor (e.g., leflunomide (4-isoxazolecarboxamide, 5-methyl-N-(4-(trifluoromethyl)phenyl)-), laflunimus (2-propenamide, 2-cyano-3-cyclopropyl-3-hydroxy-N-(3-methyl-4(trifluoromethyl)phenyl)-, (Z)-), or atovaquone (1,4-naphthalenedione, 2-(4-(4-chlorophenyl)cyclohexyl)-3-hydroxy-, trans-, or an analogue or derivative thereof).

15) IKK2 Inhibitors

In another embodiment, the pharmacologically active fibrosis-inhibiting compound is an IKK2 inhibitor (e.g., MLN-120B, SPC-839, or an analogue or derivative thereof).

16) IL-1, ICE and IRAK Antagonists

In another embodiment, the pharmacologically active fibrosis-inhibiting compound is an IL-1, ICE or an IRAK antagonist (e.g., E-5090 (2-propenoic acid, 3-(5-ethyl-4-hydroxy-3-methoxy-1-naphthalenyl)-2-methyl-, (Z)-), CH-164, CH-172, CH-490, AMG-719, iguratimod (N-(3-(formylamino)-4-oxo-6-phenoxy-4H-chromen-7-yl) methanesulfonamide), AV94-88, pralnacasan (6H-pyridazino(1,2-a)(1,2)diazepine-1-carboxamide, N-((2R,3S)-2-ethoxytetrahydro-5-oxo-3-furanyl)octahydro-9-((1-isoquinolinylcarbonyl)amino)-6,10-dioxo-, (1S,9S)—), (2S-cis)-5-(benzyloxycarbonylamino-1,2,4,5,6,7-hexahydro-4-(oxoazepino(3,2,1-hi)indole-2-carbonyl)-amino)-4-oxobutanoic acid, AVE-9488, esonarimod (benzenebutanoic acid, alpha-((acetylthio)methyl)-4-methyl-gamma-oxo-), pralnacasan (6H-pyridazino(1,2-a)(1,2)diazepine-1-carboxamide, N-((2R,3S)-2-ethoxytetrahydro-5-oxo-3-furanyl)octahydro-9-((1-isoquinolinylcarbonyl)amino)-6,10-dioxo-, (1S,9S)—), tranexamic acid (cyclohexanecarboxylic acid, 4-(aminomethyl)-, trans-), Win-72052, romazarit (Ro-31-3948) (propanoic acid, 2-((2-(4-chlorophenyl)-4-methyl-5-oxazolyl)methoxy)-2-methyl-), PD-163594, SDZ-224-015 (L-alaninamide N-((phenylmethoxy)carbonyl)-L-valyl-N-((1S)-3-((2,6-dichlorobenzoyl)oxy)-1-(2-ethoxy-2-oxoethyl)-2-oxopropyl)-), L-709049 (L-alaninamide, N-acetyl-L-tyrosyl-L-valyl-N-(2-carboxy-1-formylethyl)-, (S)—), TA-383 (1H-imidazole, 2-(4-chlorophenyl)-4,5-dihydro-4,5-diphenyl-, monohydrochloride, cis-), EI -1507-1 (6a,12a-epoxybenz(a)anthracen-1,12(2H,7H)-dione, 3,4-dihydro-3,7-dihydroxy-8-methoxy-3-methyl-), ethyl 4-(3,4-dimethoxyphenyl)-6,7-dimethoxy-2-(1,2,4-triazol-1-yl methyl)quinoline-3-carboxylate, EI-1941-1, TJ-114, anakinra (interleukin 1 receptor antagonist (human isoform x reduced), N2-L-methionyl-), IX-207-887 (acetic acid, (10-methoxy-4H-benzo(4,5)cyclohepta(1,2-b)thien-4-ylidene)-), K-832, or an analogue or derivative thereof).

17) IL-4 Agonists

In another embodiment, the pharmacologically active fibrosis-inhibiting compound is an IL-4 agonist (e.g., glatiramir acetate (L-glutamic acid, polymer with L-alanine, L-lysine and L-tyrosine, acetate (salt)), or an analogue or derivative thereof).

18) Immunomodulatory Agents

In another embodiment, the pharmacologically active fibrosis-inhibiting compound is an immunomodulatory agent (e.g., biolimus, ABT-578, methylsulfamic acid 3-(2-methoxyphenoxy)-2-(((methylamino)sulfonyl)oxy)propyl ester, sirolimus (also referred to as rapamycin or RAPAMUNE (American Home Products, Inc., Madison, N.J.)), CCI-779 (rapamycin 42-(3-hydroxy-2-(hydroxymethyl)-2-methylpropanoate)), LF-15-0195, NPC1 5669 (L-leucine, N-(((2,7-dimethyl-9H-fluoren-9-yl)methoxy)carbonyl)-), NPC-15670 (L-leucine, N-(((4,5-dimethyl-9H-fluoren-9-yl)methoxy)carbonyl)-), NPC-16570 (4-(2-(fluoren-9-yl)ethyloxy-carbonyl)aminobenzoic acid), sufosfamide (ethanol, 2-((3-(2-chloroethyl)tetrahydro-2 H-1,3,2-oxazaphosphorin-2-yl)amino)-, methanesulfonate (ester), P-oxide), tresperimus (2-(N-(4-(3-aminopropylamino)butyl)carbamoyloxy)-N-(6-guanidinohexyl)acetamide), 4-(2-(fluoren-9-yl)ethoxycarbonylamino)-benzo-hydroxamic acid, iaquinimod, PBI-1411, azathioprine (6-((1-Methyl-4-nitro-1H-imidazol-5-yl)thio)-1H-purine), PBI0032, beclometasone, MDL-28842 (9H-purin-6-amine, 9-(5-deoxy-5-fluoro-β-D-threo-pent-4-enofuranosyl)-, (Z)-), FK-788, AVE-1726, ZK-90695, ZK-90695, Ro-54864, didemnin-B, Illinois (didemnin A, N-(1-(2-hydroxy-1-oxopropyl)-L-prolyl)-, (S)—), SDZ-62-826 (ethanaminium, 2-((hydroxy((1-((octadecyloxy)carbonyl)-3-piperidinyl)methoxy)phosphinyl)oxy)-N,N,N-trimethyl-, inner salt), argyrin B ((4S,7S,13R,22R)-13-Ethyl-4-(1H-indol-3-ylmethyl)-7-(4-methoxy-1H-indol-3-ylmethyl)18,22-dimethyl-16-methyl-ene-24-thia-3,6,9,12,15,18,21,26-octaazabicyclo(21.2.1)-hexacosa-1(25),23(26)-diene-2,5,8,11,14,17,20-heptaone), everolimus (rapamycin, 42-O-(2-hydroxyethyl)-), SAR-943, L-687795, 6-((4-chlorophenyl)sulfinyl)-2,3-dihydro-2-(4-methoxy-phenyl)-5-methyl-3-oxo-4-pyridazinecarbonitrile, 91Y78 (1H-imidazo(4,5-c)pyridin-4-amine, 1-β-D-ribofuranosyl-), auranofin (gold, (1-thio-β-D-glucopyranose 2,3,4,6-tetraacetato-S)(triethylphosphine)-), 27-0-demethylrapamycin, tipredane (androsta-1,4-dien-3-one, 17-(ethylthio)-9-fluoro-11-hydroxy-17-(methylthio)-, (11 β,17 alpha)-), AI-402, LY-178002 (4-thiazolidinone, 5-((3,5-bis(1,1-dimethylethyl)-4-hydroxyphenyl)methylene)-), SM-8849 (2-thiazolamine, 4-(1-(2-fluoro(1,1′-biphenyl)-4-yl)ethyl)-N-methyl-), piceatannol, resveratrol, triamcinolone acetonide (pregna-1,4-diene-3,20-dione, 9-fluoro-11,21-dihydroxy-16,17-((1-methylethylidene)bis(oxy))-, (11 β,16 alpha)-), ciclosporin (cyclosporin A), tacrolimus (15,19-epoxy-3H-pyrido(2,1-c)(1,4)oxaazacyclotricosine-1,7,20,21 (4H,23H)-tetrone, 5,6,8,11,12,13,14,15,16,17,18,19,24,25,26,26a-hexadecahydro-5,19-dihydroxy-3-(2-(4-hydroxy-3-methoxycyclohexyl)-1-methylethenyl)-14,16-dimethoxy-4,10,12,18-tetramethyl-8-(2-propenyl)-, (3S-(3R*(E(1S*,3S*,4S*)),4S*,5R*,8S*,9E,12R*,14R*,15S*,16R*,18S*,19S*,26aR*))-), gusperimus (heptanamide, 7-((aminoiminomethyl)amino)-N-(2-((4-((3-aminopropyl)amino)butyl)amino)-1-hydroxy-2-oxoethyl)-, (±)-), tixocortol pivalate (pregn-4-ene-3,20-dione, 21-((2,2-dimethyl-1-oxopropyl)thio)-11,17-dihydroxy-, (11β)-), alefacept (1-92 LFA-3 (antigen) (human) fusion protein with immunoglobulin G1 (human hinge-CH2-CH3 gamma1-chain), dimer), halobetasol propionate (pregna-1,4-diene-3,20-dione, 21-chloro-6,9-difluoro-11-hydroxy-16-methyl-17-(1-oxopropoxy)-, (6Alpha,11β,16β)-), iloprost trometamol (pentanoic acid, 5-(hexahydro-5-hydroxy-4-(3-hydroxy-4-methyl-1-octen-6-ynyl)-2(1H)-pentalenylidene)-), beraprost (1H-cyclopenta(b)benzofuran-5-butanoic acid, 2,3,3a,8b-tetrahydro-2-hydroxy-1-(3-hydroxy-4-methyl-1-octen-6-ynyl)-), rimexolone (androsta-1,4-dien-3-one,11-hydroxy-16,17-dimethyl-17-(1-oxopropyl)-, (11β,16Alpha,17β)-), dexamethasone (pregna-1,4-diene-3,20-dione,9-fluoro-11,17,21-trihydroxy-16-methyl-, (11 β,16alpha)-), sulindac (cis-5-fluoro-2-methyl-1-((p-methylsulfinyl)benzylidene)indene-3-acetic acid), proglumetacin (1H-Indole-3-acetic acid, 1-(4-chlorobenzoyl)-5-methoxy-2-methyl-, 2-(4-(3-((4-(benzoylamino)-5-(dipropylamino)-1,5-dioxopentyl)oxy)propyl)-1-piperazinyl)ethylester, (±)-), alclometasone dipropionate (pregna-1,4-diene-3,20-dione, 7-chloro-11-hydroxy-16-methyl-17,21-bis(1-oxopropoxy)-, (7alpha, 11β,16alpha)-), pimecrolimus (15,19-epoxy-3H-pyrido(2,1-c)(1,4)oxaazacyclotricosine-1,7,20,21 (4H ,23H)-tetrone, 3-(2-(4-chloro-3-methoxycyclohexyl)-1-methyletheny)-8-ethyl-5,6,8,11,12,13,14,15,16,17,18,19,24,25,26,26a-hexadecahydro-5,19-dihydroxy-14,16-dimethoxy-4,10,12,18-tetramethyl-, (3S-(3R*(E(1S*,3S*,4R*)),4S*,5R*,8S*,9E,12R*,14R*, 15S*,16R*,18S*,19S*,26aR*))-), hydrocortisone-17-butyrate (pregn-4-ene-3,20-dione, 11,21-dihydroxy-17-(1-oxobutoxy)-, (11β)-), mitoxantrone (9,10-anthracenedione, 1,4-dihydroxy-5,8-bis((2-((2-hydroxyethyl)amino)ethyl)amino)-), mizoribine (1H-imidazole-4-carboxamide, 5-hydroxy-1-β-D-ribofuranosyl-), prednicarbate (pregna-1,4-diene-3,20-dione, 17-((ethoxycarbonyl)oxy)-11-hydroxy-21-(1-oxopropoxy)-, (11β)-), iobenzarit (benzoic acid, 2-((2-carboxyphenyl)amino)-4-chloro-), glucametacin (D-glucose, 2-(((1-(4-chlorobenzoyl)-5-methoxy-2-methyl-1H-indol-3-yl)acetyl)amino)-2-deoxy-), fluocortolone monohydrate ((6 alpha)-fluoro-16alpha-methylpregna-1,4-dien-11β,21-diol-3,20-dione), fluocortin butyl (pregna-1,4-dien-21-oic acid, 6-fluro-11-hydroxy-16-methyl-3,20-dioxo-, butyl ester, (6alpha,11β,16alpha)-), difluprednate (pregna-1,4-diene-3,20-dione, 21-(acetyloxy)-6,9-difluoro-11-hydroxy-17-(1-oxobutoxy)-, (6 alpha,11β)-), diflorasone diacetate (pregna-1,4-diene-3,20-dione, 17,21-bis(acetyloxy)-6,9-difluoro-11-hydroxy-16-methyl-, (6Alpha,11β,16β)-), dexamethasone valerate (pregna-1,4-diene-3,20-dione, 9-fluoro-11,21-dihydroxy-16-methyl-17-((1-oxopentyl)oxy)-, (11β,16Alpha)-), methylprednisolone, deprodone propionate (pregna-1,4-diene-3,20-dione, 11-hydroxy-17-(1-oxopropoxy)-, (11β)-), bucillamine (L-cysteine, N-(2-mercapto-2-methyl-1-oxopropyl)-), amcinonide (benzeneacetic acid, 2-amino-3-benzoyl-, monosodium salt, monohydrate), acemetacin (1H-indole-3-acetic acid, 1-(4-chlorobenzoyl)-5-methoxy-2-methyl-, carboxymethyl ester), or an analogue or derivative thereof).

Further, analogues of rapamycin include tacrolimus and derivatives thereof (e.g., EP0184162B1 and U.S. Pat. No. 6,258,823) everolimus and derivatives thereof (e.g., U.S. Pat. No. 5,665,772). Further representative examples of sirolimus analogues and derivatives can be found in PCT Publication Nos. WO 97/10502, WO 96/41807, WO 96/35423, WO 96/03430, WO 96/00282, WO 95/16691, WO 95/15328, WO 95/07468, WO 95/04738, WO 95/04060, WO 94/25022, WO 94/21644, WO 94/18207, WO 94/10843, WO 94/09010, WO 94/04540, WO 94/02485, WO 94/02137, WO 94/02136, WO 93/25533, WO 93/18043, WO 93/13663, WO 93/11130, WO 93/10122, WO 93/04680, WO 92/14737, and WO 92/05179. Representative U.S. patents include U.S. Pat. Nos. 6,342,507; 5,985,890; 5,604,234; 5,597,715; 5,583,139; 5,563,172; 5,561,228; 5,561,137; 5,541,193; 5,541,189; 5,534,632; 5,527,907; 5,484,799; 5,457,194; 5,457,182; 5,362,735; 5,324,644; 5,318,895; 5,310,903; 5,310,901; 5,258,389; 5,252,732; 5,247,076; 5,225,403; 5,221,625; 5,210,030; 5,208,241; 5,200,411; 5,198,421; 5,147,877; 5,140,018; 5,116,756; 5,109,112; 5,093,338; and 5,091,389.

The structures of sirolimus, everolimus, and tacrolimus are provided below:

| Name | Code Name | Company | Structure |
|---|---|---|---|
| Everolimus | SAR-943 | Novartis | See below |
| Sirolimus | AY-22989 | Wyeth | See below |
| RAPAMUNE | NSC-226080 | | |
| Rapamycin | | | |
| Tacrolimus | FK506 | Fujusawa | See below |

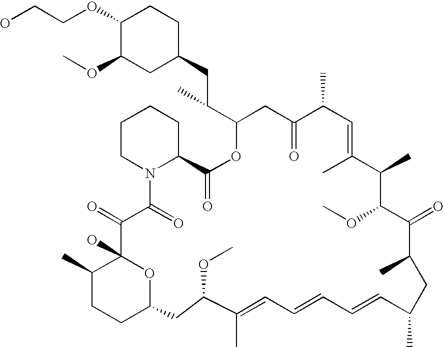

Everolimus

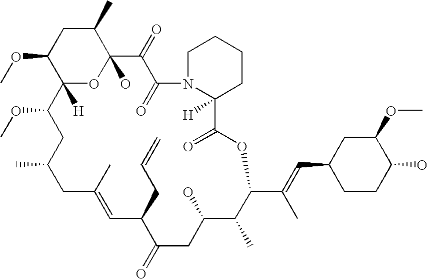

Tacrolimus

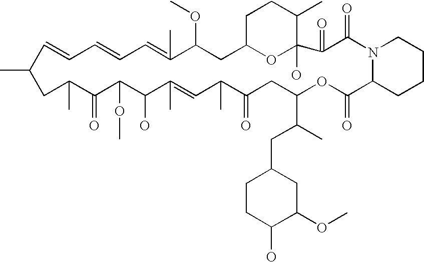

Sirolimus

Further sirolimus analogues and derivatives include tacrolimus and derivatives thereof (e.g., EP0184162B1 and U.S. Pat. No. 6,258,823) everolimus and derivatives thereof (e.g., U.S. Pat. No. 5,665,772). Further representative examples of sirolimus analogues and derivatives include ABT-578 and others may be found in PCT Publication Nos. WO 97/10502, WO 96/41807, WO 96/35423, WO 96/03430, WO 9600282, WO 95/16691, WO 9515328, WO 95/07468, WO 95/04738, WO 95/04060, WO 94/25022, WO 94/21644, WO 94/18207, WO 94/10843, WO 94/09010, WO 94/04540, WO 94/02485, WO 94/02137, WO 94/02136, WO 93/25533, WO 93/18043, WO 93/13663, WO 93/11130, WO 93/10122, WO 93/04680, WO 92/14737, and WO 92/05179. Representative U.S. patents include U.S. Pat. Nos. 6,342,507; 5,985,890; 5,604,234; 5,597,715; 5,583,139; 5,563,172; 5,561,228; 5,561,137; 5,541,193; 5,541,189; 5,534,632; 5,527,907; 5,484,799; 5,457,194; 5,457,182; 5,362,735; 5,324,644; 5,318,895; 5,310,903; 5,310,901; 5,258,389; 5,252,732; 5,247,076; 5,225,403; 5,221,625; 5,210,030; 5,208,241, 5,200,411; 5,198,421; 5,147,877; 5,140,018; 5,116,756; 5,109,112; 5,093,338; and 5,091,389.

In one aspect, the fibrosis-inhibiting agent may be, e.g., rapamycin (sirolimus), everolimus, biolimus, tresperimus, auranofin, 27-0-demethylrapamycin, tacrolimus, gusperimus, pimecrolimus, or ABT-578.

19) Inosine Monophosphate Dehydrogenase Inhibitors

In another embodiment, the pharmacologically active fibrosis-inhibiting compound is an inosine monophosphate dehydrogenase (IMPDH) inhibitor (e.g., mycophenolic acid, mycophenolate mofetil (4-hexenoic acid, 6-(1,3-dihydro-4-hydroxy-6-methoxy-7-methyl-3-oxo-5-isobenzofuranyl)-4-methyl-, 2-(4-morpholinyl)ethyl ester, (E)-), ribavirin (1H-1,2,4-triazole-3-carboxamide, 1-β-D-ribofuranosyl-), tiazofurin (4-thiazolecarboxamide, 2-β-D-ribofuranosyl-), viramidine, aminothiadiazole, thiophenfurin, tiazofurin) or an analogue or derivative thereof. Additional representative examples are included in U.S. Pat. Nos. 5,536,747, 5,807,876, 5,932,600, 6,054,472, 6,128,582, 6,344,465, 6,395,763, 6,399,773, 6,420,403, 6,479,628, 6,498,178, 6,514,979, 6,518,291, 6,541,496, 6,596,747, 6,617,323, 6,624,184, patent application Publication Nos. 2002/0040022A1, 2002/0052513A1, 2002/0055483A1, 2002/0068346A1, 2002/0111378A1, 2002/0111495A1, 2002/0123520A1, 2002/0143176A1, 2002/0147160A1, 2002/0161038A1, 2002/0173491A1, 2002/0183315A1, 2002/0193612A1, 2003/0027845A1, 2003/0068302A1, 2003/0105073A1, 2003/0130254A1, 2003/0143197A1, 2003/0144300A1, 2003/0166201A1, 2003/0181497A1, 2003/0186974A1, 2003/0186989A1, 2003/0195202A1, and PCT Publication Nos. WO 0024725A1, WO 00/25780A1, WO 00/26197A1, WO 00/51615A1, WO 00/56331A1, WO 00/73288A1, WO 01/00622A1, WO 01/66706A1, WO 01/79246A2, WO 01/81340A2, WO 01/85952A2, WO 02/16382A1, WO 02/18369A2, WO 2051814A1, WO 2057287A2, W02057425A2, WO 2060875A1, WO 2060896A1, WO 2060898A1, WO 2068058A2, WO 3020298A1, WO 3037349A1, WO 3039548A1, WO 3045901A2, WO 3047512A2, WO 3053958A1, WO 3055447A2, WO 3059269A2, WO 3063573A2, WO 3087071A1, WO 90/01545A1, WO 97/40028A1, WO 97/41211A1, WO 98/40381 A1, and WO 99/55663A1).

20) Leukotriene Inhibitors

In another embodiment, the pharmacologically active fibrosis-inhibiting compound is a leukotreine inhibitor (e.g., ONO-4057(benzenepropanoic acid, 2-(4-carboxybutoxy)-6-((6-(4-methoxyphenyl)-5-hexenyl)oxy)-, (E)-), ONO-LB-448, pirodomast 1,8-naphthyridin-2(1H)-one, 4-hydroxy-1-phenyl-3-(1-pyrrolidinyl)-, Sch-40120 (benzo(b)(1,8)naphthyridin-5(7H)-one, 10-(3-chlorophenyl)-6,8,9,10-tetrahydro-), L-656224 (4-benzofuranol, 7-chloro-2-((4-methoxyphenyl)methyl)-3-methyl-5-propyl-), MAFP (methyl arachidonyl fluorophosphonate), ontazolast (2-benzoxazolamine, N-(2-cyclohexyl-1-(2-pyridinyl)ethyl)-5-methyl-, (S)—), amelubant (carbamic acid, ((4-((3-((4-(1-(4-hydroxyphenyl)-1-methylethyl)phenoxy)methyl)phenyl)methoxy)phenyl)iminomethyl)-ethyl ester), SB-201993 (benzoic acid, 3-((((6-((1E)-2-carboxyethenyl)-5-((8-(4-methoxyphenyl)octyl)oxy)-2-pyridinyl)methyl)thio)methyl)-), LY-203647 (ethanone, 1-(2-hydroxy-3-propyl-4-(4-(2-(4-(1H-tetrazol-5-yl)butyl)-2H-tetrazol-5-yl)butoxy)phenyl)-), LY-210073, LY-223982 (benzenepropanoic acid, 5-(3-carboxybenzoyl)-2-((6-(4-methoxyphenyl)-5-hexenyl)oxy)-, (E)-), LY-293111 (benzoic acid, 2-(3-(3-((5-ethyl-4′-fluoro-2-hydroxy(1,1′-biphenyl)-4-yl)oxy)propoxy)-2-propylphenoxy)-), SM-9064 (pyrrolidine,1-(4,11-dihydroxy-13-(4-methoxyphenyl)-1-oxo-5,7,9-tridecatrienyl)-, (E,E,E)-), T-0757 (2,6-octadienamide, N-(4-hydroxy-3,5-dimethylphenyl)-3,7-dimethyl-, (2E)-), or an analogue or derivative thereof).

21) MCP-1 Antagonists

In another embodiment, the pharmacologically active fibrosis-inhibiting compound is a MCP-1 antagonist (e.g., nitronaproxen (2-napthaleneacetic acid, 6-methoxy-alpha-methyl 4-(nitrooxy)butyl ester (alpha S)—), bindarit (2-(1-benzylindazol-3-ylmethoxy)-2-methylpropanoic acid), 1-alpha-25 dihydroxy vitamin D₃, or an analogue or derivative thereof).

22) MMP Inhibitors

In another embodiment, the pharmacologically active fibrosis-inhibiting compound is a matrix metalloproteinase (MMP) inhibitor (e.g., D-9120, doxycycline (2-naphthacenecarboxamide, 4-(dimethylamino)-1,4,4a,5,5a,6,11,12a-octahydro-3,5,10,12,12a-pentahydroxy-6-methyl-1,11-dioxo-(4S-(4 alpha, 4a alpha, 5 lpha, 5a alpha, 6 alpha, 12a alpha))-), BB-2827, BB-1101 (2S-allyl-N1-hydroxy-3R-isobutyl-N4-(1S-methylcarbamoyl-2-phenylethyl)-succinamide), BB-2983, solimastat (N′-(2,2-dimethyl-1(S)-(N-(2-pyridyl)carbamoyl)propyl)-N4-hydroxy-2(R)-isobutyl-3(S)-methoxysuccinamide), batimastat (butanediamide, N4-hydroxy-N1-(2-(methylamino)-2-oxo-1-(phenylmethyl)ethyl)-2-(2-methylpropyl)-3-((2-thienylthio)methyl)-, (2R-(1(S*),2R*,3S*))-), CH-138, CH-5902, D-1927, D-5410, EF-13 (gamma-linolenic acid lithium salt),CMT-3 (2-naphthacenecarboxamide, 1,4,4a,5,5a,6,11,12a-octahydro-3,10,12,12a-tetrahydroxy-1,11-dioxo-, (4aS,5aR, 12aS)-), marimastat (N-(2,2-dimethyl-1(S)-(N-methylcarbamoyl)propyl)-N,3(S)-dihydroxy-2(R)-isobutylsuccinamide), TIMP'S,ONO-4817, rebimastat (L-Valinamide, N-((2S)-2-mercapto-1-oxo-4-(3,4,4-trimethyl-2,5-d ioxo-1-imidazolidinyl)butyl)-L-leucyl-N,3-dimethyl-), PS-508, CH-715, nimesulide (methanesulfonamide, N-(4-nitro-2-phenoxyphenyl)-), hexahydro-2-(2(R)-(1(RS)-(hydroxycarbamoyl)-4-phenylbutyl)nonanoyl)-N-(2,2,6,6-etramethyl-4-piperidinyl)-3(S)-pyridazine carboxamide, Rs-113-080, Ro-1130830, cipemastat (1-piperidinebutanamide, β-(cyclopentylmethyl)-N-hydroxy-gamma-oxo-alpha-((3,4,4-trimethyl-2,5-dioxo-1-imidazolidinyl)methyl)-,(alpha R,βR)-), 5-(4′-biphenyl)-5-(N-(4-nitrophenyl)piperazinyl)barbituric acid, 6-methoxy-1,2,3,4-tetrahydro-norharman-1-carboxylic acid, Ro-31-4724 (L-alanine, N-(2-(2-(hydroxyamino)-2-oxoethyl)-4-methyl-1-oxopentyl)-L-leucyl-, ethyl ester), prinomastat (3-thiomorpholinecarboxamide, N-hydroxy-2,2-dimethyl-4-((4-(4-pyridinyloxy) phenyl)sulfonyl)-, (3R)—), AG-3433 (1H-pyrrole-3-propanic acid, 1-(4′-cyano(1,1′-biphenyl)-4-yl)-b-((((3S)-tetrahydro-4,4-dimethyl-2-oxo-3-furanyl)amino)carbonyl)-, phenylmethyl ester, (bS)-), PNU-142769 (2H-Isoindole-2-butanamide, 1,3-dihydro-N-hydroxy-alpha-((3S)-3-(2-methylpropyl)-2-oxo-1-(2-phenylethyl)-3-pyrrolidinyl)-1,3-dioxo-, (alpha R)—), (S)-1-(2-((((4,5-dihydro-5-thioxo-1,3,4-thiadiazol-2-yl)amino)-carbonyl)amino)-1-oxo-3-(pentafluorophenyl)propyl)-4-(2-pyridinyl)piperazine, SU-5402 (1H-pyrrole-3-propanoic acid, 2-((1,2-dihydro-2-oxo-3H-indol-3-ylidene)methyl)-4-methyl-), SC-77964, PNU-171829, CGS-27023A, N-hydroxy-2(R)-((4-methoxybenzene-sulfonyl)(4-picolyl)amino)-2-(2-tetrahydrofuranyl)-acetamide, L-758354 ((1,1′-biphenyl)-4-hexanoic acid, alpha-butyl-gamma-(((2,2-dimethyl-1-((methylamino)carbonyl)propyl)amino)carbonyl)-4′-fluoro-, (alpha S-(alpha R*, gammaS*(R*)))-, GI-155704A, CPA-926, TMI-005, XL-784, or an analogue or derivative thereof). Additional representative examples are included in U.S. Pat. Nos. 5,665,777; 5,985,911; 6,288,261; 5,952,320; 6,441,189; 6,235,786; 6,294,573; 6,294,539; 6,563,002; 6,071,903; 6,358,980; 5,852,213; 6,124,502; 6,160,132; 6,197,791; 6,172,057; 6,288,086; 6,342,508; 6,228,869; 5,977,408; 5,929,097; 6,498,167; 6,534,491; 6,548,524; 5,962,481; 6,197,795; 6,162,814; 6,441,023; 6,444,704; 6,462,073; 6,162,821; 6,444,639; 6,262,080; 6,486,193; 6,329,550; 6,544,980; 6,352,976; 5,968,795; 5,789,434; 5,932,763; 6,500,847; 5,925,637; 6,225,314; 5,804,581; 5,863,915; 5,859,047; 5,861,428; 5,886,043; 6,288,063; 5,939,583; 6,166,082; 5,874,473; 5,886,022; 5,932,577; 5,854,277; 5,886,024; 6,495,565; 6,642,255; 6,495,548; 6,479,502; 5,696,082; 5,700,838; 6,444,639; 6,262,080; 6,486,193; 6,329,550; 6,544,980; 6,352,976; 5,968,795; 5,789,434; 5,932,763; 6,500,847; 5,925,637; 6,225,314; 5,804,581; 5,863,915; 5,859,047; 5,861,428; 5,886,043; 6,288,063; 5,939,583; 6,166,082; 5,874,473; 5,886,022; 5,932,577; 5,854,277; 5,886,024; 6,495,565; 6,642,255; 6,495,548; 6,479,502; 5,696,082; 5,700,838; 5,861,436; 5,691,382; 5,763,621; 5,866,717; 5,902,791; 5,962,529; 6,017,889; 6,022,873; 6,022,898; 6,103,739; 6,127,427; 6,258,851; 6,310,084; 6,358,987; 5,872,152; 5,917,090; 6,124,329; 6,329,373; 6,344,457; 5,698,706; 5,872,146; 5,853,623; 6,624,144; 6,462,042; 5,981,491; 5,955,435; 6,090,840; 6,114,372; 6,566,384; 5,994,293; 6,063,786; 6,469,020; 6,118,001; 6,187,924; 6,310,088; 5,994,312; 6,180,611; 6,110,896; 6,380,253; 5,455,262; 5,470,834; 6,147,114; 6,333,324; 6,489,324; 6,362,183; 6,372,758; 6,448,250; 6,492,367; 6,380,258; 6,583,299; 5,239,078; 5,892,112; 5,773,438; 5,696,147; 6,066,662; 6,600,057; 5,990,158; 5,731,293; 6,277,876; 6,521,606; 6,168,807; 6,506,414; 6,620,813; 5,684,152; 6,451,791; 6,476,027; 6,013,649; 6,503,892; 6,420,427; 6,300,514; 6,403,644; 6,177,466; 6,569,899; 5,594,006; 6,417,229; 5,861,510; 6,156,798; 6,387,931; 6,350,907; 6,090,852; 6,458,822; 6,509,337; 6,147,061; 6,114,568; 6,118,016; 5,804,593; 5,847,153; 5,859,061; 6,194,451; 6,482,827; 6,638,952; 5,677,282; 6,365,630; 6,130,254; 6,455,569; 6,057,369; 6,576,628; 6,110,924; 6,472,396; 6,548,667; 5,618,844; 6,495,578; 6,627,411; 5,514,716; 5,256,657; 5,773,428; 6,037,472; 6,579,890; 5,932,595; 6,013,792; 6,420,415; 5,532,265; 5,691,381; 5,639,746; 5,672,598; 5,830,915; 6,630,516; 5,324,634; 6,277,061; 6,140,099; 6,455,570; 5,595,885; 6,093,398; 6,379,667; 5,641,636; 5,698,404; 6,448,058; 6,008,220; 6,265,432; 6,169,103; 6,133,304; 6,541,521; 6,624,196; 6,307,089; 6,239,288; 5,756,545; 6,020,366; 6,117,869; 6,294,674; 6,037,361; 6,399,612; 6,495,568; 6,624,177; 5,948,780; 6,620,835; 6,284,513; 5,977,141; 6,153,612; 6,297,247; 6,559,142; 6,555,535; 6,350,885; 5,627,206; 5,665,764; 5,958,972; 6,420,408; 6,492,422; 6,340,709; 6,022,948; 6,274,703; 6,294,694; 6,531,499; 6,465,508; 6,437,177; 6,376,665; 5,268,384; 5,183,900; 5,189,178; 6,511,993; 6,617,354; 6,331,563; 5,962,466; 5,861,427; 5,830,869; and 6,087,359.

23) NF kappa B Inhibitors

In another embodiment, the pharmacologically active fibrosis-inhibiting compound is a NF kappa B (NFKB) inhibitor (e.g., AVE-0545, Oxi-104 (benzamide, 4-amino-3-chloro-N-(2-(diethylamino)ethyl)-), dexlipotam, R-flurbiprofen ((1,1′-biphenyl)-4-acetic acid, 2-fluoro-alpha-methyl), SP100030 (2-chloro-N-(3,5-di(trifluoromethyl)phenyl)-4-(trifluoromethyl)pyrimidine-5-carboxamide), AVE-0545, Viatris, AVE-0547, Bay 11-7082, Bay 11-7085, 15 deoxy-prostaylandin J2, bortezomib (boronic acid, ((1R)-3-methyl-1-(((2S)-1-oxo-3-phenyl-2-((pyrazinylcarbonyl)amino)propyl)amino)butyl)-, benzamide an d nicotinamide derivatives that inhibit NF-kappaB, such as those described in U.S. Pat. Nos. 5,561,161 and 5,340,565 (OxiGene), PG490-88Na, or an analogue or derivative thereof).

24) NO Agonists

In another embodiment, the pharmacologically active fibrosis-inhibiting compound is a NO antagonist (e.g., NCX-4016 (benzoic acid, 2-(acetyloxy)-, 3-((nitrooxy)methyl)phenyl ester, NCX-2216, L-arginine or an analogue or derivative thereof).

25) P38 MAP Kinase Inhibitors

In another embodiment, the pharmacologically active fibrosis-inhibiting compound is a p38 MAP kinase inhibitor (e.g., GW-2286, CGP-52411, BIRB-798, SB220025, RO-320-1195, RWJ-67657, RWJ-68354, SCIO-469, SCIO-323, AMG-548, CMC-146, SD-31145, CC-8866, Ro-320-1195, PD-98059 (4H-1-benzopyran-4-one, 2-(2-amino-3-methoxyphenyl)-), CGH-2466, doramapimod, SB-203580 (pyridine, 4-(5-(4-fluorophenyl)-2-(4-(methylsulfinyl)phenyl)-1H-imidazol-4-yl)-), SB-220025 ((5-(2-amino-4-pyrimidinyl)-4-(4-fluorophenyl)-1-(4-piperidinyl)imidazole), SB-281832, PD169316, SB202190, GSK-681323, EO-1606, GSK-681323, or an analogue or derivative thereof). Additional representative examples are included in U.S. Pat. Nos. 6,300,347; 6,316,464; 6,316,466; 6,376,527; 6,444,696; 6,479,507; 6,509,361; 6,579,874; 6,630,485, U.S. patent application Publication Nos. 2001/0044538A1; 2002/0013354A1; 2002/0049220A1; 2002/0103245A1; 2002/0151491A1; 2002/0156114A1; 2003/0018051A1; 2003/0073832A1; 2003/0130257A1; 2003/0130273A1; 2003/0130319A1; 2003/0139388A1; 20030139462A1; 2003/0149031 A1; 2003/0166647A1; 2003/0181411A1; and PCT Publication Nos. WO 00/63204A2; WO 01/21591A1; WO 01/35959A1; WO 01/74811A2; WO 02/18379A2; WO 2064594A2; WO 2083622A2; WO 2094842A2; WO 2096426A1; WO 2101015A2; WO 2103000A2; WO 3008413A1; WO 3016248A2; WO 3020715A1; WO 3024899A2; WO 3031431A1; W03040103A1; WO 3053940A1; WO 3053941A2; WO 3063799A2; WO 3079986A2; WO 3080024A2; WO 3082287A1; WO 97/44467A1; WO 99/01449A1; and WO 99/58523A1.

26) Phosphodiesterase Inhibitors

In another embodiment, the pharmacologically active fibrosis-inhibiting compound is a phosphodiesterase inhibitor (e.g., CDP-840 (pyridine, 4-((2R)-2-(3-(cyclopentyloxy)-4-methoxyphenyl)-2-phenylethyl)-), CH-3697, CT-2820, D-22888 (imidazo(1,5-a)pyrido(3,2-e)pyrazin-6(5H)-one, 9-ethyl-2-methoxy-7-methyl-5-propyl-), D-4418 (8-methoxyquinoline-5-(N-(2,5-dichloropyridin-3-yl))carboxamide), 1-(3-cyclopentyloxy-4-methoxyphenyl)-2-(2,6-dichloro-4-pyridyl) ethanone oxime, D-4396, ONO-6126, CDC-998, CDC-801, V-11294A (3-(3-(cyclopentyloxy)-4-methoxybenzyl)-6-(ethylamino)-8-isopropyl-3H-purine hydrochloride), S,S′-methylene-bis(2-(8-cyclopropyl-3-propyl-6-(4-pyridylmethylamino)-2-thio-3H-purine)) tetrahyrochloride, rolipram (2-pyrrolidinone, 4-(3-(cyclopentyloxy)-4-methoxyphenyl)-), CP-293121, CP-353164 (5-(3-cyclopentyloxy-4-methoxyphenyl)pyridine-2-carboxamide), oxagrelate (6-phthalazinecarboxylic acid, 3,4-dihydro-1-(hydroxymethyl)-5,7-dimethyl-4-oxo-, ethyl ester), PD-168787, ibudilast (1-propanone, 2-methyl-1-(2-(1-methylethyl)pyrazolo(1,5-a)pyridin-3-yl)-), oxagrelate (6-phthalazinecarboxylic acid, 3,4-dihydro-1-(hydroxymethyl)-5,7-dimethyl-4-oxo-, ethyl ester), griseolic acid (alpha-L-talo-oct-4-enofuranuronic acid, 1-(6-amino-9H-purin-9-yl)-3,6-anhydro-6-C-carboxy-1,5-dideoxy-), KW-4490, KS-506, T-440, roflumilast (benzamide, 3-(cyclopropylmethoxy)-N-(3,5-dichloro-4-pyridinyl)-4-(difluoromethoxy)-), rolipram, milrinone, triflusinal (benzoic acid, 2-(acetyloxy)-4-(trifluoromethyl)-), anagrelide hydrochloride (imidazo(2,1-b)quinazolin-2(3H)-one, 6,7-dichloro-1,5-dihydro-, monohydrochloride), cilostazol (2(1H)-quinolinone, 6-(4-(1-cyclohexyl-1H-tetrazol-5-yl)butoxy)-3,4-dihydro-), propentofylline (1H-purine-2,6-dione, 3,7-dihydro-3-methyl-1-(5-oxohexyl)-7-propyl-), sildenafil citrate (piperazine, 1-((3-(4,7-dihydro-1-methyl-7-oxo-3-propyl-1H-pyrazolo(4,3-d)pyrimidin-5-yl)-4-ethoxyphenyl)sulfonyl)-4-methyl, 2-hydroxy-1,2,3-propanetricarboxylate- (1:1)), tadalafil (pyrazino(1′,2′:1,6)pyrido(3,4-b)indole 1,4-dione, 6-(1,3-benzodioxol-5-yl)-2,3,6,7,12,12a-hexahydro-2-methyl-, (6R-trans)), vardenafil (piperazine, 1-(3-(1,4-dihydro-5-methyl(-4-oxo-7-propylimidazo(5,1-f)(1,2,4)-triazin-2-yl)-4-ethoxyphenyl)sulfonyl)-4-ethyl-), milrinone ((3,4′-bipyridine)-5-carbonitrile, 1,6-dihydro-2-methyl-6-oxo-), enoximone (2H-imidazol-2-one, 1,3-dihydro-4-methyl-5-(4-(methylthio)benzoyl)-), theophylline (1H-purine-2,6-dione, 3,7-dihydro-1,3-dimethyl-), ibudilast (1-propanone, 2-methyl-1-(2-(1-methylethyl)pyrazolo(1,5-a)pyridin-3-yl)-), aminophylline (1H-purine-2,6-dione, 3,7-dihydro-1,3-dimethyl-, compound with 1,2-ethanediamine (2:1)-), acebrophylline (7H-purine-7-acetic acid, 1,2,3,6-tetrahydro-1,3-dimethyl-2,6-dioxo-,compd. with trans-4-(((2-amino-3,5-dibromophenyl)methyl)amino)cyclohexanol (1:1)), plafibride (propanamide, 2-(4-chlorophenoxy)-2-methyl-N-(((4-morpholinylmethyl)amino)carbonyl)-), ioprinone hydrochloride (3-pyridinecarbonitrile, 1,2-dihydro-5-imidazo(1,2-a)pyridin-6-yl-6-methyl-2-oxo-, monohydrochloride-), fosfosal (benzoic acid, 2-(phosphonooxy)-), amrinone ((3,4′-bipyridin)-6(1H)-one, 5-amino-, or an analogue or derivative thereof).

Other examples of phosphodiesterase inhibitors include denbufylline (1H-purine-2,6-dione, 1,3-dibutyl-3,7-dihydro-7-(2-oxopropyl)-), propentofylline (1H-purine-2,6-dione, 3,7-dihydro-3-methyl-1-(5-oxohexyl)-7-propyl-) and pelrinone (5-pyrimidinecarbonitrile, 1,4-dihydro-2-methyl-4-oxo-6-((3-pyridinylmethyl)amino)-).

Other examples of phosphodiesterase III inhibitors include enoximone (2H-imidazol-2-one, 1,3-dihydro-4-methyl-5-(4-(methylthio)benzoyl)-), and saterinone (3-pyridinecarbonitrile, 1,2-dihydro-5-(4-(2-hydroxy-3-(4-(2-methoxyphenyl)-1-piperazinyl)propoxy)phenyl)-6-methyl-2-oxo-).

Other examples of phosphodiesterase IV inhibitors include AWD-12-281, 3-auinolinecarboxylic acid, 1-ethyl-6-fluoro-1,4-dihydro-7-(4-methyl-1-piperazinyl)-4-oxo-), tadalafil (pyrazino(1′,2′:1,6)pyrido(3,4-b)indolel,4-dione, 6-(1,3-benzodioxol-5-yl)-2,3,6,7,12,12a-hexahydro-2-methyl-, (6R-trans)), and filaminast (ethanone, 1-(3-(cyclopentyloxy)-4-methoxyphenyl)-, O-(aminocarbonyl)oxime, (1E)-)

Another example of a phosphodiesterase V inhibitor is vardenafil (piperazine,1-(3-(1,4-dihydro-5-methyl(-4-oxo-7-propylimidazo(5,1-f)(1,2,4)-triazin-2-yl)-4-ethoxyphenyl)sulfonyl)-4-ethyl-).

27) TGF beta Inhibitors

In another embodiment, the pharmacologically active fibrosis-inhibiting compound is a TGF beta Inhibitor (e.g., mannose-6-phosphate, LF-984, tamoxifen (ethanamine, 2-(4-(1,2-diphenyl-1-butenyl)phenoxy)-N,N-dimethyl-, (Z)-), tranilast, or an analogue or derivative thereof).

28) Thromboxane A2 Antagonists

In another embodiment, the pharmacologically active fibrosis-inhibiting compound is a thromboxane A2 antagonist (e.g., CGS-22652 (3-pyridineheptanoic acid, γ-(4-(((4-chlorophenyl)sulfonyl)amino)butyl)-, (.+−.)-), ozagrel (2-propenoic acid, 3-(4-(1H-imidazol-1-ylmethyl)phenyl)-, (E)-), argatroban (2-piperidinecarboxylic acid,1-(5-((aminoiminomethyl)amino)-1-oxo-2-(((1,2,3,4-tetrahydro-3-methyl-8-quinolinyl)sulfonyl)amino)pentyl)-4-methyl-), ramatroban (9H-carbazole-9-propanoic acid, 3-(((4-fluorophenyl)sulfonyl)amino)-1,2,3,4-tetrahydro-, (R)—), torasemide (3-pyridinesulfonamide, N-(((1-methylethyl)amino)carbonyl)-4-((3-methylphenyl)amino)-), gamma linoleic acid ((Z,Z,Z)-6,9,12-octadecatrienoic acid), seratrodast (benzeneheptanoic acid, zeta-(2,4,5-trimethyl-3,6-dioxo-1,4-cyclohexadien-1-yl)-, (±)-, or an analogue or derivative thereof).

29) TNF alpha Antagonists and TACE Inhibitors

In another embodiment, the pharmacologically active fibrosis-inhibiting compound is a TNF alpha antagonist or TACE inhibitor (e.g., E-5531 (2-deoxy-6-0-(2-deoxy-3-0-(3(R)-(5(Z)-dodecenoyloxy)-decyl)-6-0-methyl-2-(3-oxotetradecanamido)-4-O-phosphono-β-D-glucopyranosyl)-3-0-(3(R)-hydroxydecyl)-2-(3-oxotetradecanamido)-alpha-D-glucopyranose-1-O-phosphate), AZD-4717, glycophosphopeptical, UR-12715 (B=benzoic acid, 2-hydroxy-5-((4-(3-(4-(2-methyl-1H-imidazol(4,5-c)pyridin-1-yl)methyl)-1-piperidinyl)-3-oxo-1-phenyl-1-propenyl)phenyl)azo) (Z)), PMS-601, AM-87, xyloadenosine (9H-purin-6-amine, 9-β-D-xylofuranosyl-), RDP-58, RDP-59, BB2275, benzydamine, E-3330 (undecanoic acid, 2-((4,5-dimethoxy-2-methyl-3,6-dioxo-1,4-cyclohexadien-1-yl)methylene)-, (E)-), N-(D, L-2-(hydroxyaminocarbonyl)methyl-4-methylpentanoyl)-L-3-(2′-naphthyl)alanyl-L-alanine, 2-aminoethyl amide, CP-564959, MLN-608, SPC-839, ENMD-0997, Sch-23863 ((2-(10,11-dihydro-5-ethoxy-5H-dibenzo (a,d) cyclohepten-S-yl)-N,N-dimethyl-ethanamine), SH-636, PKF-241-466, PKF-242-484, TNF-484A, cilomilast (cis-4-cyano-4-(3-(cyclopentyloxy)-4-methoxyphenyl)cyclohexane-1-carboxylic acid), GW-3333, GW-4459, BMS-561392, AM-87, cloricromene (acetic acid, ((8-chloro-3-(2-(diethylamino)ethyl)-4-methyl-2-oxo-2H-1-benzopyran-7-yl)oxy)-, ethyl ester), thalidomide (1H-Isoindole-1,3(2H)-dione, 2-(2,6-dioxo-3-piperidinyl)-), vesnarinone (piperazine, 1-(3,4-dimethoxybenzoyl)-4-(1,2,3,4-tetrahydro-2-oxo-6-quinolinyl)-), infliximab, lentinan, etanercept (1-235-tumor necrosis factor receptor (human) fusion protein with 236-467-immunoglobulin G1 (human gamma1-chain Fc fragment)), diacerein (2-anthracenecarboxylic acid, 4,5-bis(acetyloxy)-9,10-dihydro-9,10-dioxo-, or an analogue or derivative thereof).

30) Tyrosine Kinase Inhibitors

In another embodiment, the pharmacologically active fibrosis-inhibiting compound is a tyrosine kinase inhibitor (e.g., SKI-606, ER-068224, SD-208, N-(6-benzothiazolyl)-4-(2-(1-piperazinyl)pyrid-5-yl)-2-pyrimidineamine, celastrol (24,25,26-trinoroleana-1(10),3,5,7-tetraen-29-oic acid, 3-hydroxy-9,13-dimethyl-2-oxo-, (9 beta., 13alpha, 14β,20 alpha)-), CP-127374 (geldanamycin, 17-demethoxy-17-(2-propenylamino)-), CP-564959, PD-1 71026, CGP-52411 (1H-Isoindole-1,3(2H)-dione, 4,5-bis(phenylamino)-), CGP-53716 (benzamide, N-(4-methyl-3-((4-(3-pyridinyl)-2-pyrimidinyl)amino)phenyl)-), imatinib (4-((methyl-1-piperazinyl)methyl)-N-(4-methyl-3-((4-(3-pyridinyl)-2-pyrimidinyl)amino)-phenyl)benzamide methanesulfonate), NVP-AAK980-NX, KF-250706 (13-chloro,5(R),6(S)-epoxy-14,16-dihydroxy-11-(hydroyimino)-3(R)-methyl-3,4,5,6,11,12-hexahydro-1H-2-benzoxacyclotetradecin-1-one), 5-(3-(3-methoxy-4-(2-((E)-2-phenylethenyl)-4-oxazolylmethoxy)phenyl)propyl)-3-(2-((E)-2-phenylethenyl)-4-oxazolylmethyl)-2,4-oxazolidinedione, genistein, NV-06, or an analogue or derivative thereof).

31) Vitronectin Inhibitors

In another embodiment, the pharmacologically active fibrosis-inhibiting compound is a vitronectin inhibitor (e.g., O-(9,10-dimethoxy-1,2,3,4,5,6-hexahydro-4-((1,4,5,6-tetrahydro-2-pyrimidinyl)hydrazono)-8-benz(e)azulenyl)-N-((phenylmethoxy)carbonyl)-DL-homoserine 2,3-dihydroxypropyl ester, (2S)-benzoylcarbonylamino-3-(2-((4S)-(3-(4,5-dihydro-1H-imidazol-2-ylamino)-propyl)-2,5-dioxo-imidazolidin-1-yl)-acetylamino)-propionate, Sch-221153, S-836, SC-68448 (β-((2-2-(((3-((aminoiminomethyl)amino)-phenyl)carbonyl)amino)acetyl)amino)-3,5-dichlorobenzenepropanoic acid), SD-7784, S-247, or an analogue or derivative thereof).

32) Fibroblast Growth Factor Inhibitors

In another embodiment, the pharmacologically active fibrosis-inhibiting compound is a fibroblast growth factor inhibitor (e.g., CT-052923 (((2H-benzo(d)1,3-dioxalan-5-methyl)amino)(4-(6,7-dimethoxyquinazolin-4-yl)piperazinyl)methane-1-thione), or an analogue or derivative thereof).

33) Protein Kinase Inhibitors

In another embodiment, the pharmacologically active fibrosis-inhibiting compound is a protein kinase inhibitor (e.g., KP-0201448, NPC15437 (hexanamide, 2,6-diamino-N-((1-(1-oxotridecyl)2-piperidinyl)methyl)-), fasudil (1H-1,4-diazepine, hexahydro-1-(5-isoquinolinylsulfonyl)-), midostaurin (benzamide, N-(2,3,10,11,12,13-hexahydro-10-methoxy-9-methyl-1-oxo-9,13-epoxy-1H,9H-diindolo(1,2,3-gh:3′,2′,1′-lm)pyrrolo(3,4-j)(1,7)benzodiazonin-11-yl)-N-methyl-, (9Alpha,10β,11β,13Alpha)-),fasudil (1H-1,4-diazepine, hexahydro-1-(5-isoquinolinylsulfonyl)-, dexniguldipine (3,5-pyridinedicarboxylic acid, 1,4-dihydro-2,6-dimethyl-4-(3-nitrophenyl)-, 3-(4,4-diphenyl-1-piperidinyl)propyl methyl ester, monohydrochloride, (R)—), LY-317615 (1H-pyrole-2,5-dione, 3-(1-methyl-1H-indol-3-yl)-4-(1-(1-(2-pyridinylmethyl)-4-piperidinyl)-1H-indol-3-yl)-, monohydrochloride), perifosine (piperidinium, 4-((hydroxy(octadecyloxy)phosphinyl)oxy)-1,1-dimethyl-, inner salt), LY-333531 (9H,18H-5,21:12,17-dimethenodibenzo(e,k)pyrrolo(3,4-h)(1,4,13)oxadiazacyclohexadecine-18,20(19H)-dione,9-((dimethylamino)methyl)-6,7,10,11-tetrahydro-, (S)—), Kynac; SPC-100270 (1,3-octadecanediol, 2-amino-, (S—(R*,R*))—), Kynacyte, or an analogue or derivative thereof).

34) PDGF Receptor Kinase Inhibitors

In another embodiment, the pharmacologically active fibrosis-inhibiting compound is a PDGF receptor kinase inhibitor (e.g., RPR-127963E, or an analogue or derivative thereof).

35) Endothelial Growth Factor Receptor Kinase Inhibitors

In another embodiment, the pharmacologically active fibrosis-inhibiting compound is an endothelial growth factor receptor kinase inhibitor (e.g., CEP-7055, SU-0879 ((E)-3-(3,5-di-tert-butyl-4-hydroxyphenyl)-2-(aminothiocarbonyl)acrylonitrile), BIBF-1000, AG-013736 (CP-868596), AMG-706, AVE-0005, NM-3 (3-(2-methylcarboxymethyl)-6-methoxy-8-hydroxy-isocoumarin), Bay-43-9006, SU-011248, or an analogue or derivative thereof).

36) Retinoic Acid Receptor Antagonists

In another embodiment, the pharmacologically active fibrosis-inhibiting compound is a retinoic acid receptor antagonist (e.g., etarotene (Ro-15-1570) (naphthalene, 6-(2-(4-(ethylsulfonyl)phenyl)-1-methylethenyl)-1,2,3,4-tetrahydro-1,1,4,4-tetramethyl-, (E)-), (2E,4E)-3-methyl-5-(2-((E)-2-(2,6,6-trimethyl-1-cyclohexen-1-yl)ethenyl)-1-cyclohexen-1-yl)-2,4-pentadienoic acid, tocoretinate (retinoic acid, 3,4-dihydro-2,5,7,8-tetramethyl-2-(4,8,12-trimethyltridecyl)-2H-1-benzopyran-6-yl ester, (2R*(4R*,8R*))-(±)-), aliretinoin (retinoic acid, cis-9, trans-13-), bexarotene (benzoic acid, 4-(1-(5,6,7,8-tetrahydro-3,5,5,8,8-pentamethyl-2-naphthalenyl)ethenyl)-), tocoretinate (retinoic acid, 3,4-dihydro-2,5,7,8-tetramethyl-2-(4,8,12-trimethyltridecyl)-2H-1-benzopyran-6-yl ester, (2R*(4R*,8R*))-(±)-, or an analogue or derivative thereof).

37) Platelet Derived Growth Factor Receptor Kinase Inhibitors

In another embodiment, the pharmacologically active fibrosis-inhibiting compound is a platelet derived growth factor receptor kinase inhibitor (e.g., leflunomide (4-isoxazolecarboxamide, 5-methyl-N-(4-(trifluoromethyl)phenyl)-, or an analogue or derivative thereof).

38) Fibrinogen Antagonists

In another embodiment, the pharmacologically active fibrosis-inhibiting compound is a fibrinogin antagonist (e.g., picotamide (1,3-benzenedicarboxamide, 4-methoxy-N,N′-bis(3-pyridinylmethyl)-, or an analogue or derivative thereof). 39) Antimycotic Agents

In another embodiment, the pharmacologically active fibrosis-inhibiting compound is an antimycotic agent (e.g., miconazole, sulconizole, parthenolide, rosconitine, nystatin, isoconazole, fluconazole, ketoconasole, imidazole, itraconazole, terpinafine, elonazole, bifonazole, clotrimazole, conazole, terconazole (piperazine, 1-(4-((2-(2,4-dichlorophenyl)-2-(1H-1,2,4-triazol-1-ylmethyl)-1,3-dioxolan-4-yl)methoxy)phenyl)-4-(1-methylethyl)-, cis-), isoconazole (1-(2-(2-6-dichlorobenzyloxy)-2-(2-,4-dichlorophenyl)ethyl)), griseofulvin (spiro(benzofuran-2(3H),1′-(2)cyclohexane)-3,4′-dione, 7-chloro-2′,4,6-trimeth-oxy-6′methyl-, (1′S-trans)-), bifonazole (1H-imidazole, 1-((1,1′-biphenyl)-4-ylphenylmethyl)-), econazole nitrate (1-(2-((4-chlorophenyl)methoxy)-2-(2,4-dichlorophenyl)ethyl)-1H-imidazole nitrate), croconazole (1H-imidazole, 1-(1-(2-((3-chlorophenyl)methoxy)phenyl)ethenyl)-), sertaconazole (1H-Imidazole, 1-(2-((7-chlorobenzo(b)thien-3-yl)methoxy)-2-(2,4-dichlorophenyl)ethyl)-), omoconazole (1H-imidazole, 1-(2-(2-(4-chlorophenoxy)ethoxy)-2-(2,4-dichlorophenyl)-1-methylethenyl)-, (Z)-), flutrimazole (1H-imidazole, 1-((2-fluorophenyl)(4-fluorophenyl)phenylmethyl)-), fluconazole (1H-1,2,4-triazole-1-ethanol, alpha-(2,4-difluorophenyl)-alpha-(1H-1,2,4-triazol-1-ylmethyl)-), neticonazole (1H-Imidazole, 1-(2-(methylthio)-1-(2-(pentyloxy)phenyl)ethenyl)-, monohydrochloride, (E)-), butoconazole (1H-imidazole, 1-(4-(4-chlorophenyl)-2-((2,6-dichlorophenyl)thio)butyl)-, (±)-), clotrimazole (1-((2-chlorophenyl)diphenylmethyl)-1H-imidazole, or an analogue or derivative thereof).

40) Bisphosphonates

In another embodiment, the pharmacologically active fibrosis-inhibiting compound is a bisphosphonate (e.g., clodronate, alendronate, pamidronate, zoledronate, or an analogue or derivative thereof). 41) Phospholipase A1 Inhibitors

In another embodiment, the pharmacologically active fibrosis-inhibiting compound is a phospholipase A1 inhibitor (e.g., ioteprednol etabonate (androsta-1,4-diene-17-carboxylic acid, 17-((ethoxycarbonyl)oxy)-11-hydroxy-3-oxo-, chloromethyl ester, (11β,17 alpha)-, or an analogue or derivative thereof).

42) Histamine H1/H2/H3 Receptor Antagonists

In another embodiment, the pharmacologically active fibrosis-inhibiting compound is a histamine H1, H2, or H3 receptor antagonist (e.g., ranitidine (1,1-ethenediamine, N-(2-(((5-((dimethylamino)methyl)-2-furanyl)methyl)thio)ethyl)-N′-methyl-2-nitro-), niperotidine (N-(2-((5-((dimethylamino)methyl)furfuryl)thio)ethyl)-2-nitro-N′-piperonyl-1,1-ethenediamine), famotidine (propanimidamide, 3-(((2-((aminoiminomethyl)amino)-4-thiazolyl)methyl)thio)-N-(aminosulfonyl)-), roxitadine acetate HCl (acetamide, 2-(acetyloxy)-N-(3-(3-(1-piperidinylmethyl)phenoxy)propyl)-, monohydrochloride), lafutidine (acetamide, 2-((2-furanylmethyl)sulfinyl)-N-(4-((4-(1-piperidinylmethyl)-2-pyridinyl)oxy)-2-butenyl)-, (Z)-), nizatadine (1,1-ethenediamine, N-(2-(((2-((dimethylamino)methyl)-4-thiazolyl)methyl)thio)ethyl)-N′-methyl-2-nitro-), ebrotidine (benzenesulfonamide, N-(((2-(((2-((aminoiminomethyl)amino)-4-thiazoly)methyl)thio)ethyl)amino)methylene)-4-bromo-), rupatadine (5H-benzo(5,6)cyclohepta(1,2-b)pyridine, 8-chloro-6,11-dihydro-11-(1-((5-methyl-3-pyridinyl)methyl)-4-piperidinylidene)-, trihydrochloride-), fexofenadine HCl (benzeneacetic acid, 4-(1-hydroxy-4-(4(hydroxydiphenylmethyl)-1-piperidinyl)butyl)-alpha, alpha-dimethyl-, hydrochloride, or an analogue or derivative thereof).

43) Macrolide Antibiotics

In another embodiment, the pharmacologically active fibrosis-inhibiting compound is a macrolide antibiotic (e.g., dirithromycin (erythromycin, 9-deoxo-11-deoxy-9,11-(imino(2-(2-methoxyethoxy)ethylidene)oxy)-, (9S(R))—), flurithromycin ethylsuccinate (erythromycin, 8-fluoro-mono(ethyl butanedioate) (ester)-), erythromycin stinoprate (erythromycin, 2′-propanoate, compound with N-acetyl-L-cysteine (1:1)), clarithromycin (erythromycin, 6-O-methyl-), azithromycin (9-deoxo-9a-aza-9a-methyl-9a-homoerythromycin-A), telithromycin (3-de((2,6-dideoxy-3-C-methyl-3-O-methyl-alpha-L-ribo-hexopyranosyl)oxy)-11,12-dideoxy-6-O-methyl-3-oxo-12,11-(oxycarbonyl((4-(4-(3-pyridinyl)-1H-imidazol-1-yl)butyl)imino))-), roxithromycin (erythromycin, 9-(O-((2-methoxyethoxy)methyl)oxime)), rokitamycin (leucomycin V, 4B-butanoate 3B-propanoate), RV-11 (erythromycin monopropionate mercaptosuccinate), midecamycin acetate (leucomycin V, 3B,9-diacetate 3,4B-dipropanoate), midecamycin (leucomycin V, 3,4B-dipropanoate), josamycin (leucomycin V, 3-acetate 4B-(3-methylbutanoate), or an analogue or derivative thereof).

44) GPIIb IIIa Receptor Antagonists

In another embodiment, the pharmacologically active fibrosis-inhibiting compound is a GPIIb IIIa receptor antagonist (e.g., tirofiban hydrochloride (L-tyrosine, N-(butylsulfonyl)-O-(4-(4-piperidinyl)butyl)-, monohydrochloride-), eptifibatide (L-cysteinamide, N6-(aminoiminomethyl)-N2-(3-mercapto-1-oxopropyl)-L-lysylglycyl-L-alpha-aspartyl-L-tryptophyl-L-prolyl-, cyclic(1→6)-disulfide), xemilofiban hydrochloride, or an analogue or derivative thereof).

45) Endothelin Receptor Antagonists

In another embodiment, the pharmacologically active fibrosis-inhibiting compound is an endothelin receptor antagonist (e.g., bosentan (benzenesulfonamide, 4-(1,1-dimethylethyl)-N-(6-(2-hydroxyethoxy)-5-(2-methoxyphenoxy)(2,2′-bipyrimidin)-4-yl)-, or an analogue or derivative thereof).

46) Peroxisome Proliferator-Activated Receptor Agonists

In another embodiment, the pharmacologically active fibrosis-inhibiting compound is a peroxisome proliferator-activated receptor agonist (e.g., gemfibrozil (pentanoic acid, 5-(2,5-dimethylphenoxy)-2,2-dimethyl-), fenofibrate (propanoic acid, 2-(4-(4-chlorobenzoyl)phenoxy)-2-methyl-, 1-methylethyl ester), ciprofibrate (propanoic acid, 2-(4-(2,2-dichlorocyclopropyl)phenoxy)-2-methyl-), rosiglitazone maleate (2,4-thiazolidinedione, 5-((4-(2-(methyl-2-pyridinylamino)ethoxy)phenyl)methyl)-, (Z)-2-butenedioate (1:1)), pioglitazone hydrochloride (2,4-thiazolidinedione, 5-((4-(2-(5-ethyl-2-pyridinyl)ethoxy)phenyl)methyl)-, monohydrochloride (±)-), etofylline clofibrate (propanoic acid, 2-(4-chlorophenoxy)-2-methyl-, 2-(1,2,3,6-tetrahydro-1,3-dimethyl-2,6-dioxo-7H-purin-7-yl)ethyl ester), etofibrate (3-pyridinecarboxylic acid, 2-(2-(4-chlorophenoxy)-2-methyl-1-oxopropoxy)ethyl ester), clinofibrate (butanoic acid, 2,2′-(cyclohexylidenebis(4,1-phenyleneoxy))bis(2-methyl-)), bezafibrate (propanoic acid, 2-(4-(2-((4-chlorobenzoyl)amino)ethyl)phenoxy)-2-methyl-), binifibrate (3-pyridinecarboxylic acid, 2-(2-(4-chlorophenoxy)-2-methyl-1-oxopropoxy)-1,3-propanediyl ester), or an analogue or derivative thereof).

In one aspect, the pharmacologically active compound is a peroxisome proliferator-activated receptor alpha agonist, such as GW-590735, GSK-677954, GSK501516, pioglitazone hydrochloride (2,4-thiazolidinedione, 5-((4-(2-(5-ethyl-2-pyridinyl)ethoxy)phenyl)methyl)-, monohydrochloride (±)-, or an analogue or derivative thereof).

47) Estrogen Receptor Agents

In another embodiment, the pharmacologically active fibrosis-inhibiting compound is an estrogen receptor agent (e.g., estradiol, 17-β-estradiol, or an analogue or derivative thereof).

48) Somatostatin Analogues

In another embodiment, the pharmacologically active fibrosis-inhibiting compound is a somatostatin analogue (e.g., angiopeptin, or an analogue or derivative thereof).

49) Neurokinin 1 Antagonists

In another embodiment, the pharmacologically active fibrosis-inhibiting compound is a neurokinin 1 antagonist (e.g., GW-597599, lanepitant ((1,4′-bipiperidine)-1′-acetamide, N-(2-(acetyl((2-methoxyphenyl)methyl)amino)-1-(1H-indol-3-ylmethyl)ethyl)-(R)—), nolpitantium chloride (1-azoniabicyclo(2.2.2)octane,1-(2-(3-(3,4-dichlorophenyl)-1-((3-(1-methylethoxy)phenyl)acetyl)-3-piperidinyl)ethyl)-4-phenyl-, chloride, (S)—), or saredutant (benzamide, N-(4-(4-(acetylamino)-4-phenyl-1-piperidinyl)-2-(3,4-dichlorophenyl)butyl)-N-methyl-, (S)—), or vofopitant (3-piperidinamine, N-((2-methoxy-5-(5-(trifluoromethyl)-1H-tetrazol-1-yl)phenyl)methyl)-2-phenyl-, (2S,3S)-, or an analogue or derivative thereof).

50) Neurokinin 3 Antagonist

In another embodiment, the pharmacologically active fibrosis-inhibiting compound is a neurokinin 3 antagonist (e.g., talnetant (4-quinolinecarboxamide, 3-hydroxy-2-phenyl-N-((1S)-1-phenylpropyl)-, or an analogue or derivative thereof).

51) Neurokinin Antagonist

In another embodiment, the pharmacologically active fibrosis-inhibiting compound is a neurokinin antagonist (e.g:, GSK-679769, GSK-823296, SR-489686 (benzamide, N-(4-(4-(acetylamino)-4-phenyl-1-piperidinyl)-2-(3,4-dichlorophenyl)butyl)-N-methyl-, (S)—), SB-223412; SB-235375 (4-quinolinecarboxamide, 3-hydroxy-2-phenyl-N-((1S)-1-phenylpropyl)-), UK-226471, or an analogue or derivative thereof).

52) VLA-4 Antagonist

In another embodiment, the pharmacologically active fibrosis-inhibiting compound is a VLA-4 antagonist (e.g., GSK683699, or an analogue or derivative thereof).

53) Osteoclast Inhibitor

In another embodiment, the pharmacologically active fibrosis-inhibiting compound is a osteoclast inhibitor (e.g., ibandronic acid (phosphonic acid, (1-hydroxy-3-(methylpentylamino)propylidene) bis-), alendronate sodium, or an analogue or derivative thereof).

54) DNA topoisomerase ATP Hydrolyzing Inhibitor

In another embodiment, the pharmacologically active fibrosis-inhibiting compound is a DNA topoisomerase ATP hydrolyzing inhibitor (e.g., enoxacin (1,8-naphthyridine-3-carboxylic acid,1-ethyl-6-fluoro-1,4-dihydro-4-oxo-7-(1-piperazinyl)-), levofloxacin (7H-Pyrido(1,2,3-de)-1,4-benzoxazine-6-carboxylic acid, 9-fluoro-2,3-dihydro-3-methyl-10-(4-methyl-1-piperazinyl)-7-oxo-, (S)—), ofloxacin (7H-pyrido(1,2,3-de)-1,4-benzoxazine-6-carboxylic acid, 9-fluoro-2,3-dihydro-3-methyl-10-(4-methyl-1-piperazinyl)-7-oxo-, (±)-), pefloxacin (3-quinolinecarboxylic acid, 1-ethyl-6-fluoro-1,4-dihydro-7-(4-methyl-1-piperazinyl)-4-oxo-), pipemidic acid (pyrido(2,3-d)pyrimidine-6-carboxylic acid, 8-ethyl-5,8-dihydro-5-oxo-2-(1-piperazinyl)-), pirarubicin (5,12-naphthacenedione, 10-((3-amino-2,3,6-trideoxy-4-O-(tetrahydro-2H-pyran-2-yl)-alpha-L-lyxo-hexopyranosyl)oxy)-7,8,9,10-tetrahydro-6,8,11-trihydroxy-8-(hydroxyacetyl )-1-methoxy-, (8S-(8 alpha,10 alpha(S*)))—), sparfloxacin (3-quinolinecarboxylic acid, 5-amino-1-cyclopropyl-7-(3,5-dimethyl-1-piperazinyl)-6,8-difluoro-1,4-dihydro-4-oxo-, cis-), AVE-6971, cinoxacin ((1,3)dioxolo(4,5-g)cinnoline-3-carboxylic acid, 1-ethyl-1,4-dihydro-4-oxo-), or an analogue or derivative thereof).

55) Angiotensin I Converting Enzyme Inhibitor

In another embodiment, the pharmacologically active fibrosis-inhibiting compound is an angiotensin I converting enzyme inhibitor (e.g., ramipril (cyclopenta(b)pyrrole-2-carboxylic acid, 1-(2-((1-(ethoxycarbonyl)-3-phenylpropyl)amino)-1-oxopropyl)octahydro-, (2S-(1(R*(R*)),2 alpha, 3aβ,6aβ))-), trandolapril (1H-indole-2-carboxylic acid, 1-(2-((1-carboxy-3-phenylpropyl)amino)-1-oxopropyl)octahydro-, (2S-(1(R*(R*)),2 alpha,3a alpha,7aβ))-), fasidotril (L-alanine, N-((2S)-3-(acetylthio)-2-(1,3-benzodioxol-5-ylmethyl)-1-oxopropyl)-, phenylmethyl ester), cilazapril (6H-pyridazino(1,2-a)(1,2)diazepine-1-carboxylic acid, 9-((1-(ethoxycarbonyl)-3-phenylpropyl)amino)octahydro-10-oxo-, (1S-(1 alpha, 9 alpha(R*)))—), ramipril (cyclopenta(b)pyrrole-2-carboxylic acid, 1-(2-((1-(ethoxycarbonyl)-3-phenylpropyl)amino)-1-oxopropyl)octahydro-, (2S-(1(R*(R*)), 2 alpha,3aβ,6aβ))-, or an analogue or derivative thereof).

56) Angiotensin II Antagonist

In another embodiment, the pharmacologically active fibrosis-inhibiting compound is an angiotensin 11 antagonist (e.g., HR-720 (1H-imidazole-5-carboxylic acid, 2-butyl-4-(methylthio)-1-((2′-((((propylamino)carbonyl)amino)sulfonyl)(1,1′-biphenyl)-4-yl)methyl)-, dipotassium salt, or an analogue or derivative thereof).

57) Enkephalinase Inhibitor

In another embodiment, the pharmacologically active fibrosis-inhibiting compound is an enkephalinase inhibitor (e.g., Aventis 100240 (pyrido(2,1-a)(2)benzazepine-4-carboxylic acid, 7-((2-(acetylthio)-1-oxo-3-phenylpropyl)amino)-1,2,3,4,6,7,8,12b-octahydro-6-oxo-, (4S-(4 alpha, 7 alpha(R*),12bβ))-), AVE-7688, or an analogue or derivative thereof).

58) Peroxisome Proliferator-Activated Receptor Gamma Agonist Insulin Sensitizer

In another embodiment, the pharmacologically active fibrosis-inhibiting compound is peroxisome proliferator-activated receptor gamma agonist insulin sensitizer (e.g., rosiglitazone maleate (2,4-thiazolidinedione, 5-((4-(2-(methyl-2-pyridinylamino)ethoxy)phenyl)methyl)-, (Z)-2-butenedioate (1:1), farglitazar (GI-262570, GW-2570, GW-3995, GW-5393, GW-9765), LY-929, LY-519818, LY-674, or LSN-862), or an analogue or derivative thereof).

59) Protein Kinase C Inhibitor

In another embodiment, the pharmacologically active fibrosis-inhibiting compound is a protein kinase C inhibitor, such as ruboxistaurin mesylate (9H,18H-5,21:12,17-dimethenodibenzo(e,k)pyrrolo(3,4-h)(1,4,13)oxadiazacyclohexadecine-18,20(19H)-dione,9-((dimethylamino)methyl)-6,7,10,11-tetrahydro-, (S)—), safingol (1,3-octadecanediol, 2-amino-, (S—(R*,R*))—), or enzastaurin hydrochloride (1H-pyrole-2,5-dione, 3-(1-methyl-1H-indol-3-yl)-4-(1-(1-(2-pyridinylmethyl)-4-piperidinyl)-1H-indol-3-yl)-, monohydrochloride), or an analogue or derivative thereof.

60) ROCK (rho-associated kinase) Inhibitors

In another embodiment, the pharmacologically active fibrosis-inhibiting compound is a ROCK (rho-associated kinase) inhibitor, such as Y-27632, HA-1077, H-1152 and 4-1-(aminoalkyl)-N-(4-pyridyl) cyclohexanecarboxamide or an analogue or derivative thereof.

61) CXCR3 Inhibitors

In another embodiment, the pharmacologically active fibrosis-inhibiting compound is a CXCR3 inhibitor such as T-487, T0906487 or analogue or derivative thereof.

62) Itk Inhibitors

In another embodiment, the pharmacologically active fibrosis-inhibiting compound is an Itk inhibitor such as BMS-509744 or an analogue or derivative thereof.

63) Cvtosolic phospholipase A₂-alpha Inhibitors

In another embodiment, the pharmacologically active fibrosis-inhibiting compound is a cytosolic phospholipase A₂-alpha inhibitor such as efipladib (PLA-902) or analogue or derivative thereof.

64) PPAR Agonist

In another embodiment, the pharmacologically active fibrosis-inhibiting compound is a PPAR Agonist (e.g., Metabolex ((-)-benzeneacetic acid, 4-chloro-alpha-(3-(trifluoromethyl)-phenoxy)-, 2-(acetylamino)ethyl ester), balaglitazone (5-(4-(3-methyl-4-oxo-3,4-dihydro-quinazolin-2-yl-methoxy)-benzyl)-thiazolidine-2,4-dione), ciglitazone (2,4-thiazolidinedione, 5-((4-((1-methylcyclohexyl)methoxy)phenyl)methyl)-), DRF-10945, farglitazar, GSK-677954, GW-409544, GW-501516, GW-590735, GW-590735, K-111, KRP-101, LSN-862, LY-519818, LY-674, LY-929, muraglitazar; BMS-298585 (Glycine, N-((4-methoxyphenoxy)carbonyl)-N-((4-(2-(5-methyl-2-phenyl-4-oxazolyl)ethoxy)phenyl)methyl)-), netoglitazone; isaglitazone (2,4-thiazolidinedione, 5-((6-((2-fluorophenyl)methoxy)-2-naphthalenyl)methyl)-), Actos AD-4833; U-72107A (2,4-thiazolidinedione, 5-((4-(2-(5-ethyl-2-pyridinyl)ethoxy)phenyl)methyl)-, monohydrochloride (±)-), JTT-501; PNU-182716 (3,5-Isoxazolid inedione, 4-((4-(2-(5-methyl-2-phenyl-4-oxazolyl)ethoxy)phenyl)methyl)-), AVANDIA (from SB Pharmco Puerto Rico, Inc. (Puerto Rico); BRL-48482;BRL-49653;BRL-49653c; NYRACTA and Venvia (both from (SmithKline Beecham (United Kingdom)); tesaglitazar ((2S)-2-ethoxy-3-(4-(2-(4-((methylsulfonyl)oxy)phenyl)ethoxy)phenyl) propanoic acid), troglitazone (2,4-Thiazolidinedione, 5-((4-((3,4-dihydro-6-hydroxy-2,5,7,8-tetramethyl-2H-1-benzopyran-2-yl)methoxy)phenyl)methyl)-), and analogues and derivatives thereof).

65) Immunosuppressants

In another embodiment, the pharmacologically active fibrosis-inhibiting compound is an immunosuppressant (e.g., batebulast (cyclohexanecarboxylic acid, 4-(((aminoiminomethyl)amino)methyl)-, 4-(1,1-dimethylethyl)phenyl ester, trans-), cyclomunine, exalamide (benzamide, 2-(hexyloxy)-), LYN-001, CCI-779 (rapamycin 42-(3-hydroxy-2-(hydroxymethyl)-2-methylpropanoate)), 1726; 1726-D; AVE-1726, or an analogue or derivative thereof).

66) Erb Inhibitor

In another embodiment, the pharmacologically active fibrosis-inhibiting compound is an Erb inhibitor (e.g., canertinib dihydrochloride (N-(4-(3-(chloro-4-fluoro-phenylamino)-7-(3-morpholin-4-yl-propoxy)-quinazolin-6-yl)-acrylamide dihydrochloride), CP-724714, or an analogue or derivative thereof).

67) Apoptosis Agonist

In another embodiment, the pharmacologically active fibrosis-inhibiting compound is an apoptosis agonist (e.g., CEFLATONIN (CGX-635) (from Chemgenex Therapeutics, Inc., Menlo Park, Calif.), CHML, LBH-589, metoclopramide (benzamide, 4-amino-5-chloro-N-(2-(diethylamino)ethyl)-2-methoxy-), patupilone (4,17-dioxabicyclo(14.1.0)heptadecane-5,9-dione, 7,11-dihydroxy-8,8,10,12,16-pentamethyl-3-(1-methyl-2-(2-methyl-4-thiazolyl)ethenyl, (1R,3S,7S,10R,11S,12S,16R)), AN-9; pivanex (butanoic acid, (2,2-dimethyl-1-oxopropoxy)methyl ester), SL-100; SL-102; SL-11093; SL-11098; SL-11099; SL-93; SL-98; SL-99, or an analogue or derivative thereof).

68) Lipocortin Agonist

In another embodiment, the pharmacologically active fibrosis-inhibiting compound is an lipocortin agonist (e.g., CGP-13774 (9Alpha-chloro-6Alpha-fluoro-11β,17alpha-dihydroxy-16Alpha-methyl-3-oxo-1,4-androstadiene-17β-carboxylic acid-methylester-17-propionate), or analogue or derivative thereof).

69) VCAM-1 Antagonist

In another embodiment, the pharmacologically active fibrosis-inhibiting compound is a VCAM-1 antagonist (e.g., DW-908e, or an analogue or derivative thereof).

70) Collagen Antagonist

In another embodiment, the pharmacologically active fibrosis-inhibiting compound is a collagen antagonist (e.g., E-5050 (Benzenepropanamide, 4-(2,6-dimethylheptyl)-N-(2-hydroxyethyl)-β-methyl-), lufironil (2,4-Pyridinedicarboxamide, N,N′-bis(2-methoxyethyl)-), or an analogue or derivative thereof).

71) Alpha 2 Integrin Antagonist

In another embodiment, the pharmacologically active fibrosis-inhibiting compound is an alpha 2 integrin antagonist (e.g., E-7820, or an analogue or derivative thereof).

72) TNF Alpha Inhibitor

In another embodiment, the pharmacologically active fibrosis-inhibiting compound is a TNF alpha inhibitor (e.g., ethyl pyruvate, Genz-29155, lentinan (Ajinomoto Co., Inc. (Japan)), linomide (3-quinolinecarboxamide, 1,2-dihydro-4-hydroxy-N,1-dimethyl-2-oxo-N-phenyl-), UR-1505, oran analogue or derivative thereof).

73) Nitric Oxide Inhibitor

In another embodiment, the pharmacologically active fibrosis-inhibiting compound is a nitric oxide inhibitor (e.g., guanidioethyldisulfide, or an analogue or derivative thereof).

74) Cathepsin Inhibitor

In another embodiment, the pharmacologically active fibrosis-inhibiting compound is a cathepsin inhibitor (e.g., SB-462795 or an analogue or derivative thereof).

Anti-Infective Agents

The present invention also provides for the combination of a polymeric composition and an agent which reduces the likelihood of infection upon implantation of the composition or a medical implant.

Infection is a common complication of the implantation of foreign bodies such as, for example, medical devices and implants. Foreign materials provide an ideal site for micro- organisms to attach and colonize. It is also hypothesized that there is an impairment of host defenses to infection in the microenvironment surrounding a foreign material. These factors make medical implants particularly susceptible to infection and make eradication of such an infection difficult, if not impossible, in most cases. In many cases, an infected implant or device must be surgically removed from the body in order to irradicate the infection.

The present invention provides agents (e.g., chemotherapeutic agents) that can be released from a composition, and which have potent antimicrobial activity at extremely low doses. A wide variety of anti-infective agents can be utilized in combination with the present compositions. Suitable anti-infective agents may be readily determined based upon the assays provided in Example 34). Discussed in more detail below are several representative examples of agents that can be used as anti-infective agents, such as: (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin).

A. Anthracyclines

In one aspect, the therapeutic anti-infective agent is an anthracycline. Anthracyclines have the following general structure, where the R groups may be a variety of organic groups:

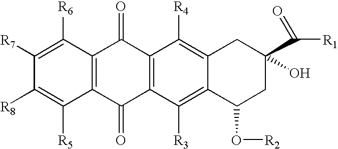

According to U.S. Pat. No. 5,594,158, suitable R groups are as follows: R₁ is CH₃ or CH₂OH; R₂ is daunosamine or H; R₃ and R₄ are independently one of OH, NO₂, NH₂, F, Cl, Br, I, CN, H or groups derived from these; R₅ is hydrogen, ydroxyl, or methoxy; and R₆₋₈ are all hydrogen. Alternatively, R₅ and R₆ are hydrogen and R₇ and R₈ are alkyl or halogen, or vice versa.

According to U.S. Pat. No. 5,843,903, R₁ may be a conjugated peptide. According to U.S. Pat. No. 4,296,105, R₅ may be an ether linked alkyl group. According to U.S. Pat. No. 4,215,062, R₅ may be OH or an ether linked alkyl group. R₁ may also be linked to the anthracycline ring by a group other than C(O), such as an alkyl or branched alkyl group having the C(O) linking moiety at its end, such as —CH₂CH(CH₂—X)C(O)-—R₁, wherein X is H or an alkyl group (see, e.g., U.S. Pat. No. 4,215,062). R₂ may alternately be a group linked by the functional group ═N—NHC(O)—Y, where Y is a group such as a phenyl or substituted phenyl ring. Alternately R₃ may have the following structure: 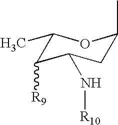
in which R₉ is OH either in or out of the plane of the ring, or is a second sugar moiety such as R₃. R₁₀ may be H or form a secondary amine with a group such as an aromatic group, saturated or partially saturated 5 or 6 membered heterocyclic having at least one ring nitrogen (see U.S. Pat. No. 5,843,903). Alternately, R₁₀ may be derived from an amino acid, having the structure —C(O)CH(NHR₁₁)(R₁₂), in which R,₁₁ is H, or forms a C₃₋₄ membered alkylene with R₁₂. R₁₂ may be H, alkyl, aminoalkyl, amino, hydroxyl, mercapto, phenyl, benzyl or methylthio (see U.S. Pat. No. 4,296,105).

Exemplary anthracyclines are doxorubicin, daunorubicin, idarubicin, epirubicin, pirarubicin, zorubicin, and carubicin. Suitable compounds have the structures:

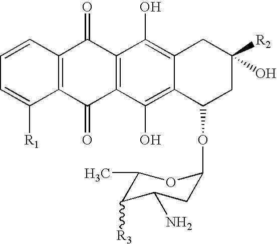

| | R₁ | R₂ | R₃ |
|---|---|---|---|
| Doxorubicin: | OCH₃ | C(O)CH₂OH | OH out of ring |
| | | | plane |
| Epirubicin: | OCH₃ | C(O)CH₂OH | OH in ring |
| (4′ epimer of | | | plane |
| doxorubicin) | | | |
| Daunorubicin: | OCH₃ | C(O)CH₃ | OH out of ring |
| | | | plane |
| Idarubicin: | H | C(O)CH₃ | OH out of ring |
| | | | plane |
| Pirarubicin: | OCH₃ | C(O)CH₂OH | 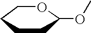 |
| Zorubicin: | OCH₃ | C(CH₃)(═N)NHC(O)C₆H₅ | OH |
| Carubicin: | OH | C(O)CH₃ | OH out of ring |
| | | | plane |

Other suitable anthracyclines are anthramycin, mitoxantrone, menogaril, nogalamycin, aclacinomycin A, olivomycin A, chromomycin A₃, and plicamycin having the structures:

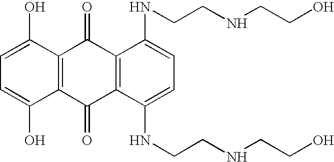

Mitoxantrone

| | R₁ | R₂ | R₃ |
|---|---|---|---|
| Menogaril | H | OCH₃ | H |
| Mogalamycin | O-sugar | H | COOCH₃ |

sugar:

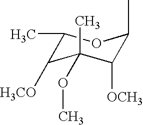

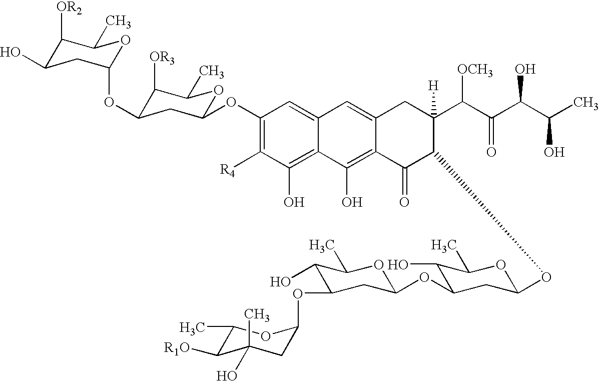

| | R₁ | R₂ | R₃ | R₄ |
|---|---|---|---|---|
| Olivomycin A | COCH(CH₃)₂ | CH₃ | COCH₃ | H |
| Chromomycin A₃ | COCH₃ | CH₃ | COCH₃ | CH₃ |
| Picamycin | H | H | H | CH₃ |

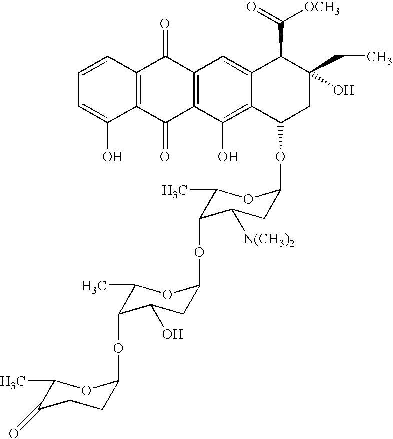

Aclacinomycin A

Other representative anthracyclines include, FCE 23762 doxorubicin derivative (Quaglia et al., J. Liq. Chromatogr. 17(18):3911-3923, 1994), annamycin (Zou et al., J. Pharm. Sci. 82(11):1151-1154, 1993), ruboxyl (Rapoport et al., J. Controlled Release 58(2):153-162, 1999), anthracycline disaccharide doxorubicin analogue (Pratesi et al., Clin. Cancer Res. 4(11):2833-2839, 1998), N-(trifluoroacetyl)doxorubicin and 4′-O-acetyl-N-(trifluoroacetyl)doxorubicin (Berube & Lepage, Synth. Commun. 28(6):1109-1116, 1998), 2-pyrrolinodoxorubicin (Nagy et al., Proc. Nat'l Acad. Sci. U.S.A. 95(4):1794-1799, 1998), disaccharide doxorubicin analogues (Arcamone et al., J. Nat'l Cancer Inst. 89(16):1217-1223, 1997), 4-demethoxy-7-O-(2,6-dideoxy-4-O-(2,3,6-trideoxy-3-amino-α-L-lyxo-hexopyranosyl)-α-L-lyxo-hexopyranosyl)adriamicinone doxorubicin disaccharide analogue (Monteagudo et al., Carbohydr. Res. 300(1):11-16, 1997), 2-pyrrolinodoxorubicin (Nagy et al., Proc. Nat'l Acad. Sci. U.S.A. 94(2):652-656, 1997), morpholinyl doxorubicin analogues (Duran et al., Cancer Chemother. Pharmacol. 38(3):210-216, 1996), enaminomalonyl-β-alanine doxorubicin derivatives (Seitz et al., Tetrahedron Lett. 36(9):1413-16, 1995), cephalosporin doxorubicin derivatives (Vrudhula et al., J. Med. Chem. 38(8):1380-5, 1995), hydroxyrubicin (Solary et al., Int. J. Cancer 58(1):85-94, 1994), methoxymorpholino doxorubicin derivative (Kuhl et al., Cancer Chemother. Pharmacol. 33(1):10-16, 1993), (6-maleimidocaproyl)hydrazone doxorubicin derivative (Willner et al., Bioconjugate Chem. 4(6):521-7, 1993), N-(5,5-diacetoxypent-1-yl) doxorubicin (Cherif & Farquhar, J. Med. Chem. 35(17):3208-14, 1992), FCE 23762 methoxymorpholinyl doxorubicin derivative (Ripamonti et al., Br. J. Cancer 65(5):703-7, 1992), N-hydroxysuccinimide ester doxorubicin derivatives (Demant et al., Biochim. Biophys. Acta 1118(1):83-90, 1991), polydeoxynucleotide doxorubicin derivatives (Ruggiero et al., Biochim. Biophys. Acta 1129(3):294-302, 1991), morpholinyl doxorubicin derivatives (EPA 434960), mitoxantrone doxorubicin analogue (Krapcho et al., J. Med. Chem. 34(8):2373-80. 1991), AD198 doxorubicin analogue (Traganos et al., Cancer Res. 51(14):3682-9, 1991), 4-demethoxy-3′-N-trifluoroacetyldoxorubicin (Horton et al., Drug Des. Delivery 6(2):123-9, 1990), 4′-epidoxorubicin (Drzewoski et al., Pol. J. Pharmacol. Pharm. 40(2):159-65, 1988; Weenen et al., Eur. J. Cancer Clin. Oncol. 20(7):919-26, 1984), alkylating cyanomorpholino doxorubicin derivative (Scudder et al., J. Nat'l Cancer Inst. 80(16):1294-8, 1988), deoxydihydroiodooxorubicin (EPA 275966), adriblastin (Kalishevskaya et al., Vestn. Mosk. Univ., 16(Biol. 1):21-7, 1988), 4′-deoxydoxorubicin (Schoeizel et al., Leuk. Res. 10(12):1455-9, 1986), 4-demethyoxy-4′-o-methyldoxorubicin (Giuliani et al., Proc. Int. Congr. Chemother. 16:285-70-285-77, 1983), 3′-deamino-3′-hydroxydoxorubicin (Horton et al., J. Antibiot. 37(8):853-8, 1984), 4-demethyoxy doxorubicin analogues (Barbieri et al., Drugs Exp. Clin. Res. 10(2):85-90, 1984), N-L-leucyl doxorubicin derivatives (Trouet et al., Anthracyclines (Proc. Int. Symp. Tumor Pharmacother.), 179-81, 1983), 3′-deamino-3′-(4-methoxy-1-piperidinyl) doxorubicin derivatives (U.S. Pat. No. 4,314,054), 3′-deamino-3′-(4-mortholinyl) doxorubicin derivatives (U.S. Pat. No. 4,301,277), 4′-deoxydoxorubicin and 4′-o-methyldoxorubicin (Giuliani et al., Int. J. Cancer 27(1):5-13, 1981), aglycone doxorubicin derivatives (Chan & Watson, J. Pharm. Sci. 67(12):1748-52, 1978), SM 5887 (Pharma Japan 1468:20, 1995), MX-2 (Pharma Japan 1420:19, 1994), 4′-deoxy-13(S)-dihydro-4′-iododoxorubicin (EP 275966), morpholinyl doxorubicin derivatives (EPA 434960), 3′-deamino-3′-(4-methoxy-1-piperidinyl) doxorubicin derivatives (U.S. Pat. No. 4,314,054), doxorubicin-14-valerate, morpholinodoxorubicin (U.S. Pat. No. 5,004,606), 3′-deamino-3′-(3″-cyano-4″-morpholinyl doxorubicin; 3′-deamino-3′-(3″-cyano-4″-morpholinyl)-13-dihydoxorubicin; (3′-deamino-3′-(3″-cyano-4″-morpholinyl) daunorubicin; 3′-deamino-3′-(3″-cyano-4″-morpholinyl)-3-dihydrodaunorubicin; and 3′-deamino-3′-(4″-morpholinyl-5-iminodoxorubicin and derivatives (U.S. Pat. No. 4,585,859), 3′-deamino-3′-(4-methoxy-1-piperidinyl) doxorubicin derivatives (U.S. Pat. No. 4,314,054) and 3-deamino-3-(4-morpholinyl) doxorubicin derivatives (U.S. Pat. No. 4,301,277).

B. Fluoropyrimidine Analogues

In another aspect, the ant-infective therapeutic agent is a fluoropyrimidine analog, such as 5-fluorouracil, or an analogue or derivative thereof, including carmofur, doxifluridine, emitefur, tegafur, and floxuridine. Exemplary compounds have the structures:

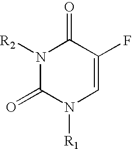

| | R₁ | R₂ |
|---|---|---|
| 5-Fluorouracil | H | H |
| Carmofur | C(O)NH(CH₂)₅CH₃ | H |
| Doxifluridine | A₁ | H |
| Floxuridine | A₂ | H |
| Emitefur | CH₂OCH₂CH₃ | B |
| Tegafur | C | H |

B

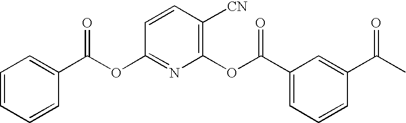

C

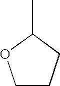

Other suitable fluoropyrimidine analogues include 5-FudR (5-fluoro-deoxyuridine), or an analogue or derivative thereof, including 5-iododeoxyuridine (5-IudR), 5-bromodeoxyuridine (5-BudR), fluorouridine triphosphate (5-FUTP), and fluorodeoxyuridine monophosphate (5-dFUMP). Exemplary compounds have the structures:

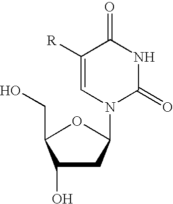

| | |
|---|---|
| 5-Fluoro-2′-deoxyuridine: | R = F |
| 5-Bromo-2′-deoxyuridine: | R = Br |
| 5-Iodo-2′-deoxyuridine: | R = I |

Other representative examples of fluoropyrimidine analogues include N3-alkylated analogues of 5-fluorouracil (Kozai et al., J. Chem. Soc., Perkin Trans. 1(19):3145-3146, 1998), 5-fluorouracil derivatives with 1,4-oxaheteroepane moieties (Gomez et al., Tetrahedron 54(43): 13295-13312, 1998), 5-fluorouracil and nucleoside analogues (Li, Anticancer Res. 17(1A):21-27, 1997), cis- and trans-5-fluoro-5,6-dihydro-6-alkoxyuracil (Van der Wilt et al., Br. J. Cancer 68(4):702-7, 1993), cyclopentane 5-fluorouracil analogues (Hronowski & Szarek, Can. J. Chem. 70(4):1162-9, 1992), A-OT-fluorouracil (Zhang et al., Zongguo Yiyao Gongye Zazhi 20(11):513-15, 1989), N4-trimethoxybenzoyl-5′-deoxy-5-fluorocytidine and 5′-deoxy-5-fluorouridine (Miwa et al., Chem. Pharm. Bull. 38(4):998-1003, 1990), 1-hexylcarbamoyl-5-fluorouracil (Hoshi et al., J. Pharmacobio-Dun. 3(9):478-81, 1980; Maehara et al., Chemotherapy (Basel) 34(6):484-9, 1988), B-3839 (Prajda et al., In Vivo 2(2):151-4, 1988), uracil-1-(2-tetrahydrofuryl)-5-fluorouracil (Anai et al., Oncology 45(3):144-7, 1988), 1-(2′-deoxy-2′-fluoro-β-D-arabinofuranosyl)-5-fluorouracil (Suzuko et al., Mol. Pharmacol. 31(3):301-6, 1987), doxifluridine (Matuura et al., Oyo Yakuri 29(5):803-31, 1985), 5′-deoxy-5-fluorouridine (Bollag & Hartmann, Eur. J. Cancer 16(4):427-32, 1980), 1-acetyl-3-O-toluyl-5-fluorouracil (Okada, Hiroshima J. Med. Sci. 28(1):49-66, 1979), 5-fluorouracil-m-formylbenzene-sulfonate (JP 55059173), N′-(2-furanidyl)-5-fluorouracil (JP 53149985) and 1-(2-tetrahydrofuryl)-5-fluorouracil (JP 52089680).

These compounds are believed to function as therapeutic agents by serving as antimetabolites of pyrimidine.

C. Folic Acid Antagonists

In another aspect, the anti-infective therapeutic agent is a folic acid antagonist, such as methotrexate or derivatives or analogues thereof, including edatrexate, trimetrexate, raltitrexed, piritrexim, denopterin, tomudex, and pteropterin. Methotrexate analogues have the following general structure: 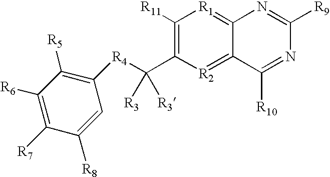
The identity of the R group may be selected from organic groups, particularly those groups set forth in U.S. Pat. Nos. 5,166,149 and 5,382,582. For example, R₁ may be N, R₂ may be N or C(CH₃), R₃′ and R₃′ may H or alkyl, e.g., CH₃, R₄ may be a single bond or NR, where R is H or alkyl group. R_{5,6,8} may be H, OCH₃, or alternately they can be halogens or hydro groups. R₇ is a side chain of the general structure: 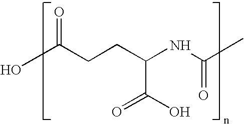
wherein n=1 for methotrexate, n=3 for pteropterin. The carboxyl groups in the side chain may be esterified or form a salt such as a Zn²⁺ salt. R₉ and R₁₀ can be NH₂ or may be alkyl substituted.

Exemplary folic acid antagonist compounds have the structures:

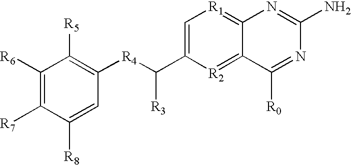

| | R₀ | R₁ | R₂ | R₃ | R₄ | R₅ | R₆ | R₇ | R₈ |
|---|---|---|---|---|---|---|---|---|---|
| Methotrexate | NH₂ | N | N | H | N(CH₃) | H | H | A (n = 1) | H |
| Edatrexate | NH₂ | N | N | H | CH(CH₂CH₃) | H | H | A (n = 1) | H |
| Trimetrexate | NH₂ | CH | C(CH₃) | H | NH | H | OCH₃ | OCH₃ | OCH₃ |
| Pteropterin | OH | N | N | H | NH | H | H | A (n = 3) | H |
| Denopterin | OH | N | N | CH₃ | N(CH₃) | H | H | A (n = 1) | H |
| Peritrexim | NH₂ | N | C(CH₃) | H | single bond | OCH₃ | H | H | OCH₃ |

A:

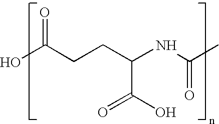

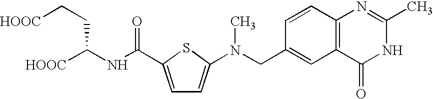

Tomudex

Other representative examples include 6-S-aminoacyloxymethyl mercaptopurine derivatives (Harada et al., Chem. Pharm. Bull. 43(10):793-6, 1995), 6-mercaptopurine (6-MP) (Kashida et al., Biol. Pharm. Bull. 18(11):1492-7, 1995), 7,8-polymethyleneimidazo-1,3,2-diazaphosphorines (Nilov et al., Mendeleev Commun. 2:67, 1995), azathioprine (Chifotides et al., J. Inorg. Biochem. 56(4):249-64, 1994), methyl-D-glucopyranoside mercaptopurine derivatives (Da Silva et al., Eur. J. Med. Chem. 29(2):149-52, 1994) and s-alkynyl mercaptopurine derivatives (Ratsino et al., Khim.-Farm. Zh. 15(8):65-7, 1981); indoline ring and a modified ornithine or glutamic acid-bearing methotrexate derivatives (Matsuoka et al., Chem. Pharm. Bull. 45(7):1146-1150, 1997), alkyl-substituted benzene ring C bearing methotrexate derivatives (Matsuoka et al., Chem. Pharm. Bull. 44(12):2287-2293, 1996), benzoxazine or benzothiazine moiety-bearing methotrexate derivatives (Matsuoka et al., J. Med. Chem. 40(1):105-111, 1997), 10-deazaaminopterin analogues (DeGraw et al., J. Med. Chem. 40(3):370-376, 1997), 5-deazaaminopterin and 5,10-dideazaaminopterin methotrexate analogues (Piper et al., J. Med. Chem. 40(3):377-384, 1997), indoline moiety-bearing methotrexate derivatives (Matsuoka et al., Chem. Pharm. Bull. 44(7):1332-1337, 1996), lipophilic amide methotrexate derivatives (Pignatello et al., World Meet. Pharm., Biopharm. Pharm. Technol., 563-4, 1995), L-threo-(2S,4S)-4-fluoroglutamic acid and DL-3,3-difluoroglutamic acid-containing methotrexate analogues (Hart et al., J. Med. Chem. 39(1):56-65, 1996), methotrexate tetrahydroquinazoline analogue (Gangjee, et al., J. Heterocycl. Chem. 32(1):243-8, 1995), N-(α-aminoacyl) methotrexate derivatives (Cheung et al., Pteridines 3(1-2):101-2, 1992), biotin methotrexate derivatives (Fan et al., Pteridines 3(1-2):131-2, 1992), D-glutamic acid or D-erythrou, threo-4-fluoroglutamic acid methotrexate analogues (McGuire et al., Biochem. Pharmacol. 42(1 2):2400-3, 1991), β,γ-methano methotrexate analogues (Rosowsky et al., Pteridines 2(3):133-9, 1991), 10-deazaaminopterin (10-EDAM) analogue (Braakhuis et al., Chem. Biol. Pteridines, Proc. Int. Symp. Pteridines Folic Acid Deriv., 1027-30, 1989), γ-tetrazole methotrexate analogue (Kalman et al., Chem. Biol. Pteridines, Proc. Int. Symp. Pteridines Folic Acid Deriv., 1154-7, 1989), N-(L-α-aminoacyl) methotrexate derivatives (Cheung et al., Heterocycles 28(2):751-8, 1989), meta and ortho isomers of aminopterin (Rosowsky et al., J. Med. Chem. 32(12):2582, 1989), hydroxymethylmethotrexate (DE 267495), γ-fluoromethotrexate (McGuire et al., Cancer Res. 49(16):4517-25, 1989), polyglutamyl methotrexate derivatives (Kumar et al., Cancer Res. 46(10):5020-3, 1986), gem-diphosphonate methotrexate analogues (WO 88/06158), α- and γ-substituted methotrexate analogues (Tsushima et al., Tetrahedron 44(17):5375-87, 1988), 5-methyl-5-deaza methotrexate analogues (U.S. Pat. No.4,725,687), Nδ-acyl-Nα-(4-amino-4-deoxypteroyl)-L-ornithine derivatives (Rosowsky et al., J. Med. Chem. 31(7):1332-7, 1988), 8-deaza methotrexate analogues (Kuehl et al., Cancer Res. 48(6):1481-8, 1988), acivicin methotrexate analogue (Rosowsky et al., J. Med. Chem. 30(8):1463-9, 1987), polymeric platinol methotrexate derivative (Carraher et al., Polym. Sci. Technol. (Plenum), 35(Adv. Biomed. Polym.):311-24, 1987), methotrexate-γ-dimyristoylphophatidylethanolamine (Kinsky et al., Biochim. Biophys. Acta 917(2):211-18, 1987), methotrexate polyglutamate analogues (Rosowsky et al., Chem. Biol. Pteridines, Pteridines Folic Acid Deriv., Proc. Int. Symp. Pteridines Folic Acid Deriv.: Chem., Biol. Clin. Aspects: 985-8, 1986), poly-γ-glutamyl methotrexate derivatives (Kisliuk et al., Chem. Biol. Pteridines, Pteridines Folic Acid Deriv., Proc. Int. Symp. Pteridines Folic Acid Deriv.: Chem., Biol. Clin. Aspects: 989-92, 1986), deoxyuridylate methotrexate derivatives (Webber et al., Chem. Biol. Pteridines, Pteridines Folic Acid Deriv., Proc. Int. Symp. Pteridines Folic Acid Deriv.: Chem., Biol. Clin. Aspects: 659-62, 1986), iodoacetyl lysine methotrexate analogue (Delcamp et al., Chem. Biol. Pteridines, Pteridines Folic Acid Deriv., Proc. Int. Symp. Pteridines Folic Acid Deriv.: Chem., Biol. Clin. Aspects: 807-9, 1986), 2,.omega.-diaminoalkanoid acid-containing methotrexate analogues (McGuire et al., Biochem. Pharmacol. 35(15):2607-13, 1986), polyglutamate methotrexate derivatives (Kamen & Winick, Methods Enzymol. 122(Vitam. Coenzymes, Pt. G):339-46, 1986), 5-methyl-5-deaza analogues (Piper et al., J. Med. Chem. 29(6):1080-7, 1986), quinazoline methotrexate analogue (Mastropaolo et al., J. Med. Chem. 29(1):155-8, 1986), pyrazine methotrexate analogue (Lever & Vestal, J. Heterocycl. Chem. 22(1):5-6, 1985), cysteic acid and homocysteic acid methotrexate analogues (U.S. Pat. No. 4,490,529), γ-tert-butyl methotrexate esters (Rosowsky et al., J. Med. Chem. 28(5):660-7, 1985), fluorinated methotrexate analogues (Tsushima et al., Heterocycles 23(1):45-9, 1985), folate methotrexate analogue (Trombe, J. Bacteriol. 160(3):849-53, 1984), phosphonoglutamic acid analogues (Sturtz & Guillamot, Eur. J. Med. Chem.—Chim. Ther. 19(3):267-73, 1984), poly (L-lysine) methotrexate conjugates (Rosowsky et al., J. Med. Chem. 27(7):888-93, 1984), dilysine and trilysine methotrexate derivates (Forsch & Rosowsky, J. Org. Chem. 49(7):1305-9, 1984), 7-hydroxymethotrexate (Fabre et al., Cancer Res. 43(10):4648-52, 1983), poly-γ-glutamyl methotrexate analogues (Piper & Montgomery, Adv. Exp. Med. Biol., 163(Folyl Antifolyl Polyglutamates):95-100, 1983), 3′,5′-dichloromethotrexate (Rosowsky & Yu, J. Med. Chem. 26(10):1448-52, 1983), diazoketone and chloromethylketone methotrexate analogues (Gangjee et al., J. Pharm. Sci. 71(6):717-19, 1982), 10-propargylaminopterin and alkyl methotrexate homologs (Piper et al., J. Med. Chem. 25(7):877-80, 1982), lectin derivatives of methotrexate (Lin et al., JNCI 66(3):523-8, 1981), polyglutamate methotrexate derivatives (Galivan, Mol. Pharmacol. 17(1):105-10, 1980), halogentated methotrexate derivatives (Fox, JNCI 58(4):J955-8, 1977), 8-alkyl-7,8-dihydro analogues (Chaykovsky et al., J. Med. Chem. 20(10):J1323-7, 1977), 7-methyl methotrexate derivatives and dichloromethotrexate (Rosowsky & Chen, J. Med. Chem. 17(12):J1308-11, 1974), lipophilic methotrexate derivatives and 3′,5′-dichloromethotrexate (Rosowsky, J. Med. Chem. 16(10):J1190-3, 1973), deaza amethopterin analogues (Montgomery et al., Ann. N.Y. Acad. Sci. 186:J227-34, 1971), MX068 (Pharma Japan, 1658:18, 1999) and cysteic acid and homocysteic acid methotrexate analogues (EPA 0142220).

These compounds are believed to act as antimetabolites of folic acid.

D. Podophyllotoxins

In another aspect, the anti-infective therapeutic agent is a Podophyllotoxin, or a derivative or an analogue thereof. Exemplary compounds of this type are etoposide or teniposide, which have the following structures:

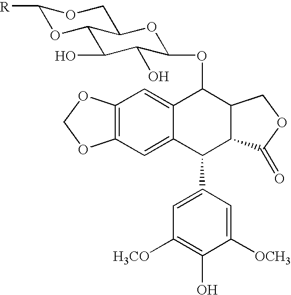

| | R |
|---|---|
| Etoposide | CH₃ |
| Teniposide | 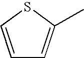 |

Other representative examples of podophyllotoxins include Cu(II)-VP-16 (etoposide) complex (Tawa et al., Bioorg. Med. Chem. 6(7):1003-1008, 1998), pyrrolecarboxamidino-bearing etoposide analogues (Ji et al., Bioorg. Med. Chem. Lett. 7(5):607-612, 1997), 4β-amino etoposide analogues (Hu, University of North Carolina Dissertation, 1992), γ-lactone ring-modified arylamino etoposide analogues (Zhou et al., J. Med. Chem. 37(2):287-92, 1994), N-glucosyl etoposide analogue (Allevi et al., Tetrahedron Lett. 34(45):7313-16, 1993), etoposide A-ring analogues (Kadow et al., Bioorg. Med. Chem. Lett. 2(1):17-22, 1992), 4′-deshydroxy-4′-methyl etoposide (Saulnier et al., Bioorg. Med. Chem. Lett. 2(10):1213-18, 1992), pendulum ring etoposide analogues (Sinha et al., Eur. J. Cancer 26(5):590-3, 1990) and E-ring desoxy etoposide analogues (Saulnier et al., J. Med. Chem. 32(7):1418-20, 1989).

These compounds are believed to act as topoisomerase II inhibitors and/or DNA cleaving agents.

E. Camptothecins

In another aspect, the anti-infective therapeutic agent is camptothecin, or an analogue or derivative thereof. Camptothecins have the following general structure.

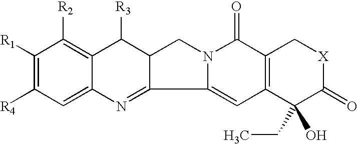

In this structure, X is typically O, but can be other groups, e.g., NH in the case of 21-lactam derivatives. R₁ is typically H or OH, but may be other groups, e.g., a terminally hydroxylated C₁₋₃ alkane. R₂ is typically H or an amino containing group such as (CH₃)₂NHCH₂, but may be other groups e.g., NO₂, NH₂, halogen (as disclosed in, e.g., U.S. Pat. No. 5,552,156) or a short alkane containing these groups. R₃ is typically H or a short alkyl such as C₂H₅. R₄ is typically H but may be other groups, e.g., a methylenedioxy group with R₁.

Exemplary camptothecin compounds include topotecan, irinotecan (CPT-11), 9-aminocamptothecin, 21-lactam-20(S)-camptothecin, 10,11-methylenedioxycamptothecin, SN-38, 9-nitrocamptothecin, 10-hydroxycamptothecin. Exemplary compounds have the structures:

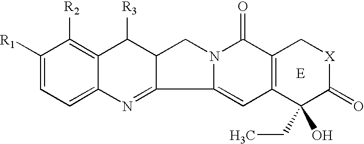

| | R₁ | R₂ | R₃ |
|---|---|---|---|
| Camptothecin: | H | H | H |
| Topotecan: | OH | (CH₃)₂NHCH₂ | H |
| SN-38: | OH | H | C₂H₅ |

X: O for most analogs, NH for 21-lactam analogs

Camptothecins have the five rings shown here. The ring labeled E must be intact (the lactone rather than carboxylate form) for maximum activity and minimum toxicity.

Camptothecins are believed to function as topoisomerase I inhibitors and/or DNA cleavage agents.

F. Hydroxyureas

The anti-infective therapeutic agent of the present invention may be a hydroxyurea. Hydroxyureas have the following general structure:

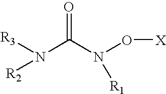

Suitable hydroxyureas are disclosed in, for example, U.S. Pat. No. 6,080,874, wherein R₁ is: 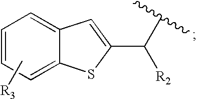
and R₂ is an alkyl group having 1-4 carbons and R₃ is one of H, acyl, methyl, ethyl, and mixtures thereof, such as a methylether.

Other suitable hydroxyureas are disclosed in, e.g., U.S. Pat. No. 5,665,768, wherein R₁ is a cycloalkenyl group, for example N-(3-(5-(4-fluorophenylthio)-furyl)-2-cyclopenten-1-yl)N-hydroxyurea; R₂ is H or an alkyl group having 1 to 4 carbons and R₃ is H; X is H or a cation.

Other suitable hydroxyureas are disclosed in, e.g., U.S. Pat. No. 4,299,778, wherein R₁ is a phenyl group substituted with one or more fluorine atoms; R₂ is a cyclopropyl group; and R₃ and X is H.

Other suitable hydroxyureas are disclosed in, e.g., U.S. Pat. No. 5,066,658, wherein R₂ and R₃ together with the adjacent nitrogen form: 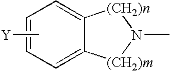
where in m is 1 or 2, n is 0-2 and Y is an alkyl group.

In one aspect, the hydroxyurea has the structure:

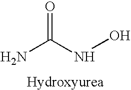

These compounds are thought to function by inhibiting DNA synthesis.

G. Platinum Complexes

In another aspect, the anti-infective therapeutic agent is a platinum compound. In general, suitable platinum complexes may be of Pt(II) or Pt(IV) and have this basic structure: 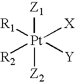
wherein X and Y are anionic leaving groups such as sulfate, phosphate, carboxylate, and halogen; R₁ and R₂ are alkyl, amine, amino alkyl any may be further substituted, and are basically inert or bridging groups. For Pt(II) complexes Z₁ and Z₂ are non-existent. For Pt(IV) Z₁ and Z₂ may be anionic groups such as halogen, hydroxy, carboxylate, ester, sulfate or phosphate. See, e.g., U.S. Pat. Nos. 4,588,831 and 4,250,189.

Suitable platinum complexes may contain multiple Pt atoms. See, e.g., U.S. Pat. Nos. 5,409,915 and 5,380,897. For example bisplatinum and triplatinum complexes of the type:

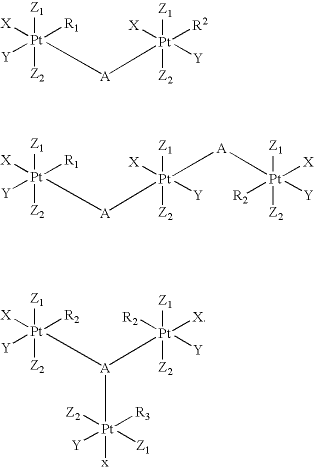

Exemplary platinum compounds are cisplatin, carboplatin, oxaliplatin, and miboplatin having the structures:

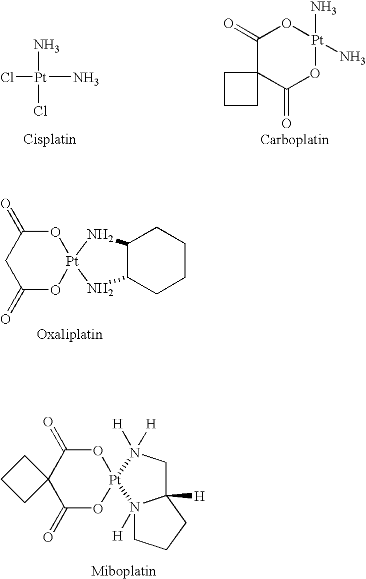

Other representative platinum compounds include (CPA)₂Pt(DOLYM) and (DACH)Pt(DOLYM) cisplatin (Choi et al., Arch. Pharmacal Res. 22(2):151-156, 1999), Cis-(PtCl₂(4,7-H-5-methyl-7-oxo)1,2,4(triazolo(1,5-a)pyrimidine)₂) (Navarro et al., J. Med. Chem. 41(3):332-338, 1998), (Pt(cis-1,4-DACH)(trans-Cl₂)(CBDCA)).½MeOH cisplatin (Shamsuddin et al., Inorg. Chem. 36(25):5969-5971, 1997), 4-pyridoxate diammine hydroxy platinum (Tokunaga et al., Pharm. Sci. 3(7):353-356, 1997), Pt(II) . . . Pt(II) (Pt₂(NHCHN(C(CH₂)(CH₃)))₄) (Navarro et al., Inorg. Chem. 35(26):7829-7835, 1996), 254-S cisplatin analogue (Koga et al., Neurol. Res. 18(3):244-247, 1996), o-phenylenediamine ligand bearing cisplatin analogues (Koeckerbauer & Bednarski, J. Inorg. Biochem. 62(4):281-298, 1996), trans, cis-(Pt(OAc)₂I₂(en)) (Kratochwil et al., J. Med. Chem. 39(13):2499-2507, 1996), estrogenic 1,2-diarylethylenediamine ligand (with sulfur-containing amino acids and glutathione) bearing cisplatin analogues (Bednarski, J. Inorg. Biochem. 62(1):75, 1996), cis-1,4-diaminocyclohexane cisplatin analogues (Shamsuddin et al., J. Inorg. Biochem. 61(4):291-301, 1996), 5′ orientational isomer of cis-(Pt(NH₃)(4-aminoTEMP-O){d(GpG)})(Dunham & Lippard, J. Am. Chem. Soc. 117(43):10702-12, 1995), chelating diamine-bearing cisplatin analogues (Koeckerbauer & Bednarski, J. Pharm. Sci. 84(7):819-23, 1995), 1,2-diarylethyleneamine ligand-bearing cisplatin analogues (Otto et al., J. Cancer Res. Clin. Oncol. 121(1):31-8, 1995), (ethylenediamine)platinum(II) complexes (Pasini et al., J. Chem. Soc., Dalton Trans. 4:579-85, 1995), CI-973 cisplatin analogue (Yang et al., Int. J. Oncol. 5(3):597-602, 1994), cis-diaminedichloroplatinum(II) and its analogues cis-1,1-cyclobutanedicarbosylato(2R)-2-methyl-1,4-butanediamineplatinum(II) and cis-diammine(glycolato)platinum (Claycamp & Zimbrick, J. Inorg. Biochem. 26(4):257-67, 1986; Fan et al., Cancer Res. 48(11):3135-9, 1988; Heiger-Bernays et al., Biochemistry 29(36):8461-6, 1990; Kikkawa et al., J. Exp. Clin. Cancer Res. 12(4):233-40, 1993; Murray et al., Biochemistry 31(47):11812-17, 1992; Takahashi et al., Cancer Chemother. Pharmacol. 33(1 ):31-5, 1993), cis-amine-cyclohexylamine-dichloroplatinum(II) (Yoshida et al., Biochem. Pharmacol. 48(4):793-9, 1994), gem-diphosphonate cisplatin analogues (FR 2683529), (meso-1,2-bis(2,6-dichloro-4-hydroxyplenyl)ethylenediamine) dichloroplatinum(II) (Bednarski et al., J. Med. Chem. 35(23):4479-85, 1992), cisplatin analogues containing a tethered dansyl group (Hartwig et al., J. Am. Chem. Soc. 114(21):8292-3, 1992), platinum(II) polyamines (Siegmann et al., Inorg. Met.-Containing Polym. Mater., (Proc. Am. Chem. Soc. Int. Symp.), 335-61, 1990), cis-(3H)dichloro(ethylenediamine)platinum(II) (Eastman, Anal. Biochem. 197(2):311-15, 1991), trans-diamminedichloroplatinum(II) and cis-(Pt(NH₃)₂(N₃-cytosine)Cl) (Bellon & Lippard, Biophys. Chem. 35(2-3):179-88, 1990), 3H-cis-1,2-diaminocyclohexanedichloroplatinum(II) and 3H-cis-1,2-diaminocyclohexane-malonatoplatinum (II) (Oswald et al., Res. Commun. Chem. Pathol. Pharmacol. 64(1):41-58, 1989), diaminocarboxylatoplatinum (EPA 296321), trans-(D,1)-1,2-diaminocyclohexane carrier ligand-bearing platinum analogues (Wyrick & Chaney, J. Labelled Compd. Radiopharm. 25(4):349-57, 1988), aminoalkylaminoanthraquinone-derived cisplatin analogues (Kitov et al., Eur. J. Med. Chem. 23(4):381-3, 1988), spiroplatin, carboplatin, iproplatin and JM40 platinum analogues (Schroyen et al., Eur. J. Cancer Clin. Oncol. 24(8):1309-12, 1988), bidentate tertiary diamine-containing cisplatinum derivatives (Orbell et al., Inorg. Chim. Acta 152(2):125-34, 1988), platinum(II), platinum(IV) (Liu & Wang, Shandong Yike Daxue Xuebao 24(1):35-41, 1986), cis-diammine(1,1-cyclobutanedicarboxylato-)platinum(II) (carboplatin, JM8) and ethylenediammine-malonatoplatinum(II) (JM40) (Begg et al., Radiother. Oncol. 9(2):157-65, 1987), JM8 and JM9 cisplatin analogues (Harstrick et al., Int. J. Androl. 10(1); 139-45, 1987), (NPr4)2((PtCL4).cis-(PtCl2-(NH2Me)2)) (Brammer et al., J. Chem. Soc., Chem. Commun. 6:443-5, 1987), aliphatic tricarboxylic acid platinum complexes (EPA 185225), and cis-dichloro(amino acid)(tert-butylamine)platinum(II) complexes (Pasini & Bersanetti, Inorg. Chim. Acta 107(4):259-67, 1985). These compounds are thought to function by binding to DNA, i.e., acting as alkylating agents of DNA.

Dosages of Anti-Infective Agents

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ M to 10⁻⁷ M, or about 10⁻⁷ M to 10⁻⁶ M about 10⁻⁶ M to 10⁻⁵ M or about 10⁻⁴ M of the agent is maintained on the tissue surface.

(a) Anthracyclines. Utilizing the anthracycline doxorubicin as an example, whether applied as a polymer coating, incorporated into the polymers which make up the implant components, or applied without a carrier polymer, the total dose of doxorubicin applied to the device or implant should not exceed 25 mg (range of 0.1 μg to 25 mg). In a particularly preferred embodiment, the total amount of drug applied should be in the range of 1 μg to 5 mg. The dose per unit area (i.e., the amount of drug as a function of the surface area of the portion of the implant to which drug is applied and/or incorporated) should fall within the range of 0.01 μg-100 μg per mm² of surface area. In a particularly preferred embodiment, doxorubicin should be applied to the implant surface at a dose of 0.1 μg/mm²-10 μg/mm². As different polymer and non-polymer coatings will release doxorubicin at differing rates, the above dosing parameters should be utilized in combination with the release rate of the drug from the implant surface such that a minimum concentration of 10⁻⁷-10⁻⁴ M of doxorubicin is maintained on the surface. It is necessary to insure that surface drug concentrations exceed concentrations of doxorubicin known to be lethal to multiple species of bacteria and fungi (i.e., are in excess of 10⁻⁴ M; although for some embodiments lower concentrations are sufficient). In a preferred embodiment, doxorubicin is released from the surface of the implant such that anti-infective activity is maintained for a period ranging from several hours to several months. In a particularly preferred embodiment the drug is released in effective concentrations for a period ranging from 1 week-6 months. It should be readily evident based upon the discussions provided herein that analogues and derivatives of doxorubicin (as described previously) with similar functional activity can be utilized for the purposes of this invention; the above dosing parameters are then adjusted according to the relative potency of the analogue or derivative as compared to the parent compound (e.g., a compound twice as potent as doxorubicin is administered at half the above parameters, a compound half as potent as doxorubicin is administered at twice the above parameters, etc.).

Utilizing mitoxantrone as another example of an anthracycline, whether applied as a polymer coating, incorporated into the polymers which make up the device or implant, or applied without a carrier polymer, the total dose of mitoxantrone applied should not exceed 5 mg (range of 0.01 μg to 5 mg). In a particularly preferred embodiment, the total amount of drug applied should be in the range of 0.1 μg to 1 mg. The dose per unit area (i.e., the amount of drug as a function of the surface area of the portion of the implant to which drug is applied and/or incorporated) should fall within the range of 0.01 μg-20 μg per mm² of surface area. In a particularly preferred embodiment, mitoxantrone should be applied to the implant surface at a dose of 0.05 μg/mm²-3 μg/mm². As different polymer and non-polymer coatings will release mitoxantrone at differing rates, the above dosing parameters should be utilized in combination with the release rate of the drug from the implant surface such that a minimum concentration of 10⁻⁵-10⁻⁶ M of mitoxantrone is maintained. It is necessary to insure that drug concentrations on the implant surface exceed concentrations of mitoxantrone known to be lethal to multiple species of bacteria and fungi (i.e., are in excess of 10⁻⁵ M; although for some embodiments lower drug levels will be sufficient). In a preferred embodiment, mitoxantrone is released from the surface of the implant such that anti-infective activity is maintained for a period ranging from several hours to several months. In a particularly preferred embodiment the drug is released in effective concentrations for a period ranging from 1 week-6 months. It should be readily evident based upon the discussions provided herein that analogues and derivatives of mitoxantrone (as described previously) with similar functional activity can be utilized for the purposes of this invention; the above dosing parameters are then adjusted according to the relative potency of the analogue or derivative as compared to the parent compound (e.g., a compound twice as potent as mitoxantrone is administered at half the above parameters, a compound half as potent as mitoxantrone is administered at twice the above parameters, etc.).

(b) Fluoropyrimidines Utilizing the fluoropyrimidine 5-fluorouracil as an example, whether applied as a polymer coating, incorporated into the polymers which make up the device or implant, or applied without a carrier polymer, the total dose of 5-fluorouracil applied should not exceed 250 mg (range of 1.0 μg to 250 mg). In a particularly preferred embodiment, the total amount of drug applied should be in the range of 10 μg to 25 mg. The dose per unit area (i.e., the amount of drug as a function of the surface area of the portion of the implant to which drug is applied and/or incorporated) should fall within the range of 0.1 μg-1 mg per mm² of surface area. In a particularly preferred embodiment, 5-fluorouracil should be applied to the implant surface at a dose of 1.0 μg/mm²-50 μ/mm². As different polymer and non-polymer coatings will release 5-fluorouracil at differing rates, the above dosing parameters should be utilized in combination with the release rate of the drug from the implant surface such that a minimum concentration of 10⁻⁴-10⁻⁷ M of 5-fluorouracil is maintained. It is necessary to insure that surface drug concentrations exceed concentrations of 5-fluorouracil known to be lethal to numerous species of bacteria and fungi (i.e., are in excess of 10⁻⁴ M; although for some embodiments lower drug levels will be sufficient). In a preferred embodiment, 5-fluorouracil is released from the implant surface such that anti-infective activity is maintained for a period ranging from several hours to several months. In a particularly preferred embodiment the drug is released in effective concentrations for a period ranging from 1 week-6 months. It should be readily evident based upon the discussions provided herein that analogues and derivatives of 5-fluorouracil (as described previously) with similar functional activity can be utilized for the purposes of this invention; the above dosing parameters are then adjusted according to the relative potency of the analogue or derivative as compared to the parent compound (e.g., a compound twice as potent as 5-fluorouracil is administered at half the above parameters, a compound half as potent as 5-fluorouracil is administered at twice the above parameters, etc.).

(c) Podophylotoxins Utilizing the podophylotoxin etoposide as an example, whether applied as a polymer coating, incorporated into the polymers which make up the device or implant, or applied without a carrier polymer, the total dose of etoposide applied should not exceed 25 mg (range of 0.1 μg to 25 mg). In a particularly preferred embodiment, the total amount of drug applied should be in the range of 1 μg to 5 mg. The dose per unit area (i.e., the amount of drug as a function of the surface area of the portion of the implant to which drug is applied and/or incorporated) should fall within the range of 0.01 μg-100 μg per mm² of surface area. In a particularly preferred embodiment, etoposide should be applied to the implant surface at a dose of 0.1 μg/mm²-10 μg/mm². As different polymer and non-polymer coatings will release etoposide at differing rates, the above dosing parameters should be utilized in combination with the release rate of the drug from the implant surface such that a concentration of 10⁻⁵-10⁻⁶ M of etoposide is maintained. It is necessary to insure that surface drug concentrations exceed concentrations of etoposide known to be lethal to a variety of bacteria and fungi (i.e., are in excess of 10⁻⁵ M; although for some embodiments lower drug levels will be sufficient). In a preferred embodiment, etoposide is released from the surface of the implant such that anti-infective activity is maintained for a period ranging from several hours to several months. In a particularly preferred embodiment the drug is released in effective concentrations for a period ranging from 1 week-6 months. It should be readily evident based upon the discussions provided herein that analogues and derivatives of etoposide (as described previously) with similar functional activity can be utilized for the purposes of this invention; the above dosing parameters are then adjusted according to the relative potency of the analogue or derivative as compared to the parent compound (e.g., a compound twice as potent as etoposide is administered at half the above parameters, a compound half as potent as etoposide is administered at twice the above parameters, etc.).

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) can be utilized to enhance the antibacterial activity of the composition.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

Combination Therapies

In addition to incorporation of the above-mentioned therapeutic agents (i.e., anti-infective agents or fibrosis-inhibiting agents), one or more other pharmaceutically active agents can be incorporated into the present compositions to improve or enhance efficacy. In one aspect, the composition may further include a compound which acts to have an inhibitory effect on pathological processes in or around the treatment site. Representative examples of additional therapeutically active agents include, by way of example and not limitation, anti-thrombotic agents, anti-proliferative agents, anti-inflammatory agents, neoplastic agents, enzymes, receptor antagonists or agonists, hormones, antibiotics, antimicrobial agents, antibodies, cytokine inhibitors, IMPDH (inosine monophosplate dehydrogenase) inhibitors tyrosine kinase inhibitors, MMP inhibitors, p38 MAP kinase inhibitors, immunosuppressants, apoptosis antagonists, caspase inhibitors, and JNK inhibitor

The polymeric composition may further include an anti-thrombotic agent and/or antiplatelet agent and/or a thrombolytic agent, which reduces the likelihood of thrombotic events upon implantation of a medical implant. Representative examples of anti-thrombotic and/or antiplatelet and/or thrombolytic agents include heparin, heparin fragments, organic salts of heparin, heparin complexes (e.g., benzalkonium heparinate, tridodecylammonium heparinate), dextran, sulfonated carbohydrates such as dextran sulfate, coumadin, coumarin, heparinoid, danaparoid, argatroban chitosan sulfate, chondroitin sulfate, danaparoid, lepirudin, hirudin, AMP, adenosine, 2-chloroadenosine, acetylsalicylic acid, phenylbutazone, indomethacin, meclofenamate, hydrochloroquine, dipyridamole, iloprost, streptokinase, factor Xa inhibitors, such as DX9065a, magnesium, and tissue plasminogen activator. Further examples include plasminogen, lys-plasminogen, alpha-2-antiplasmin, urokinase, aminocaproic acid, ticlopidine, clopidogrel, trapidil (triazolopyrimidine), naftidrofuryl, auriritricarboxylic acid and glycoprotein IIb/IIIa inhibitors such as abcixamab, eptifibatide, and tirogiban. Other agents capable of affecting the rate of clotting include glycosaminoglycans, danaparoid, 4-hydroxycourmarin, warfarin sodium, dicumarol, phenprocoumon, indan-1,3-dione, acenocoumarol, anisindione, and rodenticides including bromadiolone, brodifacoum, diphenadione, chlorophacinone, and pidnone.

The polymeric formulation may be or include a hydrophilic polymer gel that itself has anti-thrombogenic properties. For example, the composition can be in the form of a coating that can comprise a hydrophilic, biodegradable polymer that is physically removed from the surface of the device over time, thus reducing adhesion of platelets to the device surface. The gel composition can include a polymer or a blend of polymers. Representative examples include alginates, chitosan and chitosan sulfate, hyaluronic acid, dextran sulfate, PLURONIC polymers (e.g., F-127 or F87), chain extended PLURONIC polymers, various polyester-polyether block copolymers of various configurations (e.g., AB, ABA, or BAB, where A is a polyester such as PLA, PGA, PLGA, PCL or the like), examples of which include MePEG-PLA, PLA-PEG-PLA, and the like). In one embodiment, the anti-thrombotic composition can include a crosslinked gel formed from a combination of molecules (e.g., PEG) having two or more terminal electrophilic groups and two or more nucleophilic groups.

The polymeric formulation may further include an agent from one of the following classes of compounds: anti-inflammatory agents (e.g., dexamethasone, cortisone, fludrocortisone, prednisone, prednisolone, 6α-methylprednisolone, triamcinolone, betamethasone, and aspirin); MMP inhibitors (e.g., batimistat, marimistat, TIMP's representative examples of which are included in U.S. Pat. Nos. 5,665,777; 5,985,911; 6,288,261; 5,952,320; 6,441,189; 6,235,786; 6,294,573; 6,294,539; 6,563,002; 6,071,903; 6,358,980; 5,852,213; 6,124,502; 6,160,132; 6,197,791; 6,172,057; 6,288,086; 6,342,508; 6,228,869; 5,977,408; 5,929,097; 6,498,167; 6,534,491; 6,548,524; 5,962,481; 6,197,795; 6,162,814; 6,441,023; 6,444,704; 6,462,073; 6,162,821; 6,444,639; 6,262,080; 6,486,193; 6,329,550; 6,544,980; 6,352,976; 5,968,795; 5,789,434; 5,932,763; 6,500,847; 5,925,637; 6,225,314; 5,804,581; 5,863,915; 5,859,047; 5,861,428; 5,886,043; 6,288,063; 5,939,583; 6,166,082; 5,874,473; 5,886,022; 5,932,577; 5,854,277; 5,886,024; 6,495,565; 6,642,255; 6,495,548; 6,479,502; 5,696,082; 5,700,838; 6,444,639; 6,262,080; 6,486,193; 6,329,550; 6,544,980; 6,352,976; 5,968,795; 5,789,434; 5,932,763; 6,500,847; 5,925,637; 6,225,314; 5,804,581; 5,863,915; 5,859,047; 5,861,428; 5,886,043; 6,288,063; 5,939,583; 6,166,082; 5,874,473; 5,886,022; 5,932,577; 5,854,277; 5,886,024; 6,495,565; 6,642,255; 6,495,548; 6,479,502; 5,696,082; 5,700,838; 5,861,436; 5,691,382; 5,763,621; 5,866,717; 5,902,791; 5,962,529; 6,017,889; 6,022,873; 6,022,898; 6,103,739; 6,127,427; 6,258,851; 6,310,084; 6,358,987; 5,872,152; 5,917,090; 6,124,329; 6,329,373; 6,344,457; 5,698,706; 5,872,146; 5,853,623; 6,624,144; 6,462,042; 5,981,491; 5,955,435; 6,090,840; 6,114,372; 6,566,384; 5,994,293; 6,063,786; 6,469,020; 6,118,001; 6,187,924; 6,310,088; 5,994,312; 6,180,611; 6,110,896; 6,380,253; 5,455,262; 5,470,834; 6,147,114; 6,333,324; 6,489,324; 6,362,183; 6,372,758; 6,448,250; 6,492,367; 6,380,258; 6,583,299; 5,239,078; 5,892,112; 5,773,438; 5,696,147; 6,066,662; 6,600,057; 5,990,158; 5,731,293; 6,277,876; 6,521,606; 6,168,807; 6,506,414; 6,620,813; 5,684,152; 6,451,791; 6,476,027; 6,013,649; 6,503,892; 6,420,427; 6,300,514; 6,403,644; 6,177,466; 6,569,899; 5,594,006; 6,417,229; 5,861,510; 6,156,798; 6,387,931; 6,350,907; 6,090,852; 6,458,822; 6,509,337; 6,147,061; 6,114,568; 6,118,016; 5,804,593; 5,847,153; 5,859,061; 6,194,451; 6,482,827; 6,638,952; 5,677,282; 6,365,630; 6,130,254; 6,455,569; 6,057,369; 6,576,628; 6,110,924; 6,472,396; 6,548,667; 5,618,844; 6,495,578; 6,627,411; 5,514,716; 5,256,657; 5,773,428; 6,037,472; 6,579,890; 5,932,595; 6,013,792; 6,420,415; 5,532,265; 5,639,746; 5,672,598; 5,830,915; 6,630,516; 5,324,634; 6,277,061; 6,140,099; 6,455,570; 5,595,885; 6,093,398; 6,379,667; 5,641,636; 5,698,404; 6,448,058; 6,008,220; 6,265,432; 6,169,103; 6,133,304; 6,541,521; 6,624,196; 6,307,089; 6,239,288; 5,756,545; 6,020,366; 6,117,869; 6,294,674; 6,037,361; 6,399,612; 6,495,568; 6,624,177; 5,948,780; 6,620,835; 6,284,513; 5,977,141; 6,153,612; 6,297,247; 6,559,142; 6,555,535; 6,350,885; 5,627,206; 5,665,764; 5,958,972; 6,420,408; 6,492,422; 6,340,709; 6,022,948; 6,274,703; 6,294,694; 6,531,499; 6,465,508; 6,437,177; 6,376,665; 5,268,384; 5,183,900; 5,189,178; 6,511,993; 6,617,354; 6,331,563; 5,962,466; 5,861,427; 5,830,869; and 6,087,359), cytokine inhibitors (chlorpromazine, mycophenolic acid, rapamycin, 1α-hydroxy vitamin D₃), IMPDH (inosine monophosplate dehydrogenase) inhibitors (e.g., mycophenolic acid, ribaviran, aminothiadiazole, thiophenfurin, tiazofurin, viramidine) (Representative examples are included in U.S. Pat., Nos. 5,536,747; 5,807,876; 5,932,600; 6,054,472; 6,128,582; 6,344,465; 6,395,763; 6,399,773; 6,420,403; 6,479,628; 6,498,178; 6,514,979; 6,518,291; 6,541,496; 6,596,747; 6,617,323; and 6,624,184, U.S. patent application Nos. 2002/0040022A1, 2002/0052513A1, 2002/0055483A1, 2002/0068346A1; 2002/0111378A1, 2002/0111495A1, 2002/0123520A1, 2002/0143176A1, 2002/0147160A1, 2002/0161038A1, 2002/0173491A1, 2002/0183315A1, 2002/0193612A1, 2003/0027845A1, 2003/0068302A1, 2003/0105073A1, 2003/0130254A1, 2003/0143197A1, 2003/0144300A1, 2003/0166201A1, 2003/0181497A1, 2003/0186974A1, 2003/0186989A1, and 2003/0195202A1, and PCT Publication Nos. WO 00/24725A1, WO 00/25780A1, WO 00/26197A1, WO 00/51615A1, WO 00/56331A1, WO 00/73288A1, WO 01/00622A1, WO 01/66706A1, WO 01/79246A2, WO 01/81340A2, WO 01/85952A2, WO 02/16382A1, WO 02/18369A2, WO 02/051814A1, WO 02/057287A2, WO 02/057425A2, WO 02/060875A1, WO 02/060896A1, WO 02/060898A1, WO 02/068058A2, WO 03/020298A1, WO 03/037349A1, WO 03/039548A1, WO 03/045901A2, WO 03/047512A2, WO 03/053958A1, WO 03/055447A2, WO 03/059269A2, WO 03/063573A2, WO 03/087071A1, WO 99/001545A1, WO 97/40028A1, WO 97/41211A1, WO 98/40381A1, and WO 99/55663A1), p38 MAP kinase inhibitors (MAPK) (e.g., GW-2286, CGP-52411, BIRB-798, SB220025, RO-320-1195, RWJ-67657, RWJ-68354, SCIO-469) (Representative examples are included in U.S. Pat. Nos. 6,300,347; 6,316,464; 6,316,466; 6,376,527; 6,444,696; 6,479,507; 6,509,361; 6,579,874, and 6,630,485, and U.S. patent application Publication Nos. 2001/0044538A1, 2002/0013354A1, 2002/0049220A1, 2002/0103245A1, 2002/0151491A1, 2002/0156114A1, 2003/0018051A1, 2003/0073832A1, 2003/0130257A1, 2003/0130273A1, 2003/0130319A1, 2003/0139388A1, 2003/0139462A1, 2003/0149031A1, 2003/0166647A1, and 2003/0181411A1, and PCT Publication Nos. WO 00/63204A2, WO 01/21591A1, WO 01/35959A1, WO 01/74811A2, WO 02/18379A2, WO 02/064594A2, WO 02/083622A2, WO 02/094842A2,WO 02/096426A1, WO 02/101015A2, WO 02/103000A2, WO 03/008413A1, WO 03/016248A2, WO 03/020715A1, WO 03/024899A2, WO 03/031431A1, WO 03/040103A1, WO 03/053940A1, WO 03/053941A2, WO 03/063799A2, WO 03/079986A2, WO 03/080024A2, WO 03/082287A1, WO 97/44467A1, WO 99/01449A1, and WO 99/58523A1), and immunomodulatory agents (rapamycin, everolimus, ABT-578, azathioprine azithromycin, analogues of rapamycin, including tacrolimus and derivatives thereof (e.g., EP 0184162B1 and those described in U.S. Pat. No. 6,258,823) and everolimus and derivatives thereof (e.g., U.S. Pat. No. 5,665,772). Further representative examples of sirolimus analogues and derivatives include ABT-578 and those found in PCT Publication Nos. WO 97/10502, WO 96/41807, WO 96/35423, WO 96/03430, WO 96/00282, WO 95/16691, WO 95/15328, WO 95/07468, WO 95/04738, WO 95/04060, WO 94/25022, WO 94/21644, WO 94/18207, WO 94/10843, WO 94/09010, WO 94/04540, WO 94/02485, WO 94/02137, WO 94/02136, WO 93/25533, WO 93/18043, WO 93/13663, WO 93/11130, WO 93/10122, WO 93/04680, WO 92/14737, and WO 92/05179 and in U.S. Pat. Nos. 6,342,507; 5,985,890; 5,604,234; 5,597,715; 5,583,139; 5,563,172; 5,561,228; 5,561,137; 5,541,193; 5,541,189; 5,534,632; 5,527,907; 5,484,799; 5,457,194; 5,457,182; 5,362,735; 5,324,644; 5,318,895; 5,310,903; 5,310,901; 5,258,389; 5,252,732; 5,247,076; 5,225,403; 5,221,625; 5,210,030; 5,208,241; 5,200,411; 5,198,421; 5,147,877; 5,140,018; 5,116,756; 5,109,112; 5,093,338; and 5,091,389.

Other examples of biologically active agents which may be included in the compositions of the invention include tyrosine kinase inhibitors, such as imantinib, ZK-222584, CGP-52411, CGP-53716, NVP-AAK980-NX, CP-1 27374, CP-564959, PD-171026, PD-173956, PD-180970, SU-0879, and SKI-606; MMP inhibitors such as nimesulide, PKF-241-466, PKF-242-484, CGS-27023A, SAR-943, primomastat, SC-77964, PNU-171829, AG-3433, PNU-142769, SU-5402, and Dexlipotam; p38 MAP kinase inhibitors such as include CGH-2466 and PD-98-59; immunosuppressants such as argyrin B, macrocyclic lactone, ADZ-62-826, CCI-779, tilomisole, amcinonide, FK-778, AVE-1726, and MDL-28842; cytokine inhibitors such as TNF-484A, PD-172084, CP-293121, CP-353164, and PD-168787; NFKB inhibitors, such as, AVE-0547, AVE-0545, and IPL-576092; HMGCoA reductase inhibitors, such as, pravestatin, atorvastatin, fluvastatin, dalvastatin, glenvastatin, pitavastatin, CP-83101, U-20685; apoptosis antagonist (e.g., troloxamine, TCH-346 (N-methyl-N-propargyl-10-aminomethyl-dibenzo(b,f)oxepin); and caspase inhibitors (e.g., PF-5901 (benzenemethanol, alpha-pentyl-3-(2-quinolinylmethoxy)-), and JNK inhibitor (e.g., AS-602801).

In another aspect, the composition may further include an antibiotic (e.g., amoxicillin, trimethoprim-sulfamethoxazole, azithromycin, clarithromycin, amoxicillin-clavulanate, cefprozil, cefuroxime, cefpodoxime, or cefdinir).

In certain aspects, a polymeric composition comprising a fibrosis-inhibiting agent is combined with an agent that can modify metabolism of the agent in vivo to enhance efficacy of the fibrosis-inhibiting agent. One class of therapeutic agents that can be used to alter drug metabolism includes agents capable of inhibiting oxidation of the anti-scarring agent by cytochrome P450 (CYP). In one embodiment, compositions are provided that include a fibrosis-inhibiting agent (e.g., paclitaxel, rapamycin, everolimus) and a CYP inhibitor, which may be combined (e.g., coated) with any of the devices described herein, including, without limitation, stents, grafts, patches, valves, wraps, and films. Representative examples of CYP inhibitors include flavones, azole antifungals, macrolide antibiotics, HIV protease inhibitors, and anti-sense oligomers. Devices comprising a combination of a fibrosis-inhibiting agent and a CYP inhibitor may be used to treat a variety of proliferative conditions that can lead to undesired scarring of tissue, including intimal hyperplasia, surgical adhesions, and tumor growth.

In another aspect, a polymeric composition comprising an anti-infective agent (e.g., anthracyclines (e.g., doxorubicin or mitoxantrone), fluoropyrimidines (e.g., 5-fluorouracil), folic acid antagonists (e.g., methotrexate and/or podophylotoxins (e.g., etoposide)) can be combined with traditional antibiotic and/or antifungal agents to enhance efficacy. The anti-infective agent may be further combined with anti-thrombotic and/or antiplatelet agents (for example, heparin, dextran sulfate, danaparoid, lepirudin, hirudin, AMP, adenosine, 2-chloroadenosine, aspirin, phenylbutazone, indomethacin, meclofenamate, hydrochloroquine, dipyridamole, iloprost, ticlopidine, clopidogrel, abcixamab, eptifibatide, tirofiban, streptokinase, and/or tissue plasminogen activator) to enhance efficacy.

Although the above therapeutic agents have been provided for the purposes of illustration, it should be understood that the present invention is not so limited. For example, although agents are specifically referred to above, the present invention should be understood to include analogues, derivatives and conjugates of such agents. As an illustration, paclitaxel should be understood to refer to not only the common chemically available form of paclitaxel, but analogues (e.g., TAXOTERE, as noted above) and paclitaxel conjugates (e.g., paclitaxel-PEG, paclitaxel-dextran, or paclitaxel-xylos). In addition, as will be evident to one of skill in the art, although the agents set forth above may be noted within the context of one class, many of the agents listed in fact have multiple biological activities. Further, more than one therapeutic agent may be utilized at a time (i.e., in combination), or delivered sequentially.

H. Compositions and Methods for Generating Compositions which Comprise a Therapeutic Agent

The present invention provides various compositions which can be used to inhibit fibrosis and/or infection of tissue in the vicinity of a treatment site (e.g., a surgical site). Within various embodiments, fibrosis and/or infection is inhibited by local or systemic release of specific pharmacological agents that become localized at the site or intervention. Within other embodiments, fibrosis and/or infection can be inhibited by local or systemic release of specific pharmacological agents that become localized adjacent to a device or implant that has been introduced into a host. In certain embodiments, compositions are provided which inhibit fibrosis in and around an implanted device, or prevent “stenosis” of a device/implant in situ, thus enhancing the efficacy. In other embodiments, anti-infective compositions are provided which inhibit or prevent infection in and around an implanted device.

There are numerous methods available for optimizing delivery of the therapeutic agent to the site of the intervention. Several of these are described below.

1) Systemic, Regional and Local Delivery of Therapeutic Agents

A variety of drug-delivery technologies are available for systemic, regional and local delivery of anti-infective and/or anti-fibrosis therapeutic agents.

For systemic delivery of therapeutic agents, several routes of administration would be suitable to provide systemic exposure of the therapeutic agent, including: (a) intravenous, (b) oral, (c) subcutaneous, (d) intraperitoneal, (e) intrathecal, (f) inhaled and intranasal, (g) sublingual or transbuccal, (h) rectal, (i) intravaginal, (j) intra-arterial, (k) intracardiac, (l) transdermal, (m) intra-ocular and (n) intramuscular. The therapeutic agent may be administered as a sustained low dose therapy to prevent disease progression, prolong disease remission, or decrease symptoms in active disease. Alternatively, the therapeutic agent may be administered in higher doses as a “pulse” therapy to induce remission in acutely active disease. The minimum dose capable of achieving these endpoints can be used and can vary according to patient, severity of disease, formulation of the administered agent, potency and tolerability of the therapeutic agent, and route of administration.

For regional and local delivery of therapeutic agents, several techniques would be suitable to achieve preferentially elevated levels of therapeutic agents in the vicinity of the area to be treated. These include: (a) using drug-delivery catheters and/or a syringe and needle for local, regional or systemic delivery of fibrosis-inhibiting agents to the tissue surrounding the device or implant (typically, drug delivery catheters are advanced through the circulation or inserted directly into tissues under radiological guidance until they reach the desired anatomical location; the fibrosis-inhibiting agent can then be released from the catheter lumen in high local concentrations in order to deliver therapeutic doses of the drug to the tissue surrounding the device or implant); (b) drug localization techniques such as magnetic, ultrasonic or MRI-guided drug delivery; (c) chemical modification of the therapeutic drug or formulation designed to increase uptake of the agent into damaged tissues (e.g., antibodies directed against damaged or healing tissue components such as macrophages, neutrophils, smooth muscle cells, fibroblasts, extracellular matrix components, neovascular tissue); (d) chemical modification of the therapeutic drug or formulation designed to localize the drug to areas of bleeding or disrupted vasculature; and/or (e) direct injection, for example subcutaneous, intramuscular, intra-articular, etc, of the therapeutic agent, for example, under normal or endoscopic vision.

2) Infiltration of Therapeutic Agents into the Tissue Surrounding a Device or Implant

Alternatively, the tissue cavity or surgical pocket into which a device or implant is placed can be treated with an anti-infective and/or fibrosis-inhibiting therapeutic agent prior to, during, or after the procedure. This can be accomplished in several ways including: (a) topical application of the agent into the anatomical space or surface where the device will be placed (particularly useful for this embodiment is the use of polymeric carriers which release the agent over a period ranging from several hours to several weeks. Compositions that can be used for this application include, e.g., fluids, microspheres, pastes, gels, microparticulates, sprays, aerosols, solid implants and other formulations which release a therapeutic agent into the region where the device or implant will be implanted); (b) microparticulate forms of the therapeutic agent are also useful for directed delivery into the implantation site; (c) sprayable collagen-containing formulations such as COSTASIS and crosslinked derivatized poly(ethylene glycol)-collagen compositions (described, e.g., in U.S. Pat. Nos. 5,874,500 and 5,565,519 and referred to herein as “CT3” (both from Angiotech Pharmaceuticals, Inc., Canada), either alone, or loaded with a therapeutic agent, applied to the implantation site (or the implant/device surface); (d) sprayable PEG-containing formulations such as COSEAL or ADHIBIT (Angiotech Pharmaceuticals, Inc.), SPRAYGEL or DURASEAL (both from Confluent Surgical, Inc., Boston, Mass.), either alone, or loaded with a therapeutic agent, applied to the implantation site (or the implant/device surface); (e) fibrin-containing formulations such as FLOSEAL or TISSEEL (both from Baxter Healthcare Corporation, Fremont, Calif.), applied to the implantation site (or the implant/device surface); (f) hyaluronic acid-containing formulations such as RESTYLANE or PERLANE (both from Q-Med AB, Sweden), HYLAFORM (Inamed Corporation (Santa Barbara, Calif.)), SYNVISC (Biomatrix, Inc., Ridgefield, N.J.), SEPRAFILM or SEPRACOAT (both from Genzyme Corporation, Cambridge, Mass.) loaded with a therapeutic agent applied to the implantation site (or the implant/device surface); (g) polymeric gels for surgical implantation such as REPEL (Life Medical Sciences, Inc., Princeton, N.J.) or FLOGEL (Baxter Healthcare Corporation) loaded with a therapeutic agent applied to the implantation site (or the implant/device surface); (h) orthopedic “cements” used to hold prostheses and tissues in place with a therapeutic agent applied to the implantation site (or the implant/device surface); (i) surgical adhesives containing cyanoacrylates such as DERMABOND (Johnson & Johnson, Inc., New Brunswick, N.J.), INDERMIL (U.S. Surgical Company, Norwalk, Conn.), GLUSTITCH (Blacklock Medical Products Inc., Canada), TISSUMEND II (Veterniary Products Laboratories, Phoenix, Ariz.), VETBOND (3M Company, St. Paul, Minn.), HISTOACRYL BLUE (Davis & Geck, St. Louis, Mo.) and ORABASE SMOOTHE-N-SEAL Liquid Protectant (Colgate-Palmolive Company, New York, N.Y.) loaded with a therapeutic agent, applied to the implantation site (or the implant/device surface); and/or (j) protein-based sealants or adhesives such as BIOGLUE (Cryolife, Inc.) and TISSUEBOND (TissueMed, Ltd.) loaded with a therapeutic agent, applied to the implantation site (or the implant/device surface).

A preferred polymeric matrix which can be used to help prevent the formation of fibrous tissue, either alone or in combination with a fibrosis inhibiting agent/composition, is formed from reactants comprising either one or both of pentaerythritol poly(ethylene glycol)ether tetra-sulfhydryl](4-armed thiol PEG, which includes structures having a linking group(s) between a sulfhydryl group(s) and the terminus of the polyethylene glycol backbone) and pentaerythritol poly(ethylene glycol)ether tetra-succinimidyl glutarate](4-armed NHS PEG, which again includes structures having a linking group(s) between a NHS group(s) and the terminus of the polyethylene glycol backbone) as reactive reagents. Another preferred composition comprises either one or both of pentaerythritol poly(ethylene glycol)ether tetra-amino](4-armed amino PEG, which includes structures having a linking group(s) between an amino group(s) and the terminus of the polyethylene glycol backbone) and pentaerythritol poly(ethylene glycol)ether tetra-succinimidyl glutarate](4-armed NHS PEG, which again includes structures having a linking group(s) between a NHS group(s) and the terminus of the polyethylene glycol backbone) as reactive reagents. Chemical structures for these reactants are shown in, e.g., U.S. Pat. No. 5,874,500. Optionally, collagen or a collagen derivative (e.g., methylated collagen) is added to the poly(ethylene glycol)-containing reactant(s) to form a preferred crosslinked matrix that can serve as a polymeric carrier for a therapeutic agent or a stand-alone composition to help prevent the formation of fibrous tissue.

3) Sustained-Release Preparations of Therapeutic Agents

As described previously, desired therapeutic agents may be admixed with, blended with, conjugated to, or, otherwise modified to contain a polymer composition (which may be either biodegradable or non-biodegradable) or a non-polymeric composition in order to release the therapeutic agent over a prolonged period of time. For many of the aforementioned embodiments, localized delivery as well as localized sustained delivery of the fibrosis-inhibiting and/or anti-infective agent may be required. For example, a desired therapeutic agent may be admixed with, blended with, conjugated to, or, otherwise modified to contain a polymeric composition (which may be either biodegradable or non-biodegradable) or non-polymeric composition in order to release the therapeutic agent over a period of time.

Representative examples of biodegradable polymers suitable for the delivery of the aforementioned therapeutic agents include albumin, collagen, gelatin, hyaluronic acid, starch, cellulose and cellulose derivatives (e.g., regenerated cellulose, methylcellulose, hydroxypropylcellulose, hydroxypropylmethylcellulose, carboxymethylcellulose, cellulose acetate phthalate, cellulose acetate succinate, hydroxypropylmethylcellulose phthalate), casein, dextrans, polysaccharides, fibrinogen, poly(ether ester) multiblock copolymers, based on poly(ethylene glycol) and poly(butylene terephthalate), tyrosine-derived polycarbonates (e.g., U.S. Pat. No. 6,120,491), poly(hydroxyl acids), poly(D,L-lactide), poly(D,L-lactide-co-glycolide), poly(glycolide), poly(hydroxybutyrate), polydioxanone, poly(alkylcarbonate) and poly(orthoesters), polyesters, poly(hydroxyvaleric acid), polydioxanone, polyesters, poly(malic acid), poly(tartronic acid), poly(acrylamides), polyanhydrides, polyphosphazenes, poly(amino acids), poly(alkylene oxide)-poly(ester) block copolymers (e.g., X—Y, X—Y—X, Y—X—Y, R—(Y—X)ₙ, or R—(X—Y)ₙ, where X is a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLGA, PLA, PCL, polydioxanone and copolymers thereof) and R is a multifunctional initiator), and the copolymers as well as blends thereof (see generally, Illum, L., Davids, S. S. (eds.) “Polymers in Controlled Drug Delivery” Wright, Bristol, 1987; Arshady, J. Controlled Release 17:1-22, 1991; Pitt, Int. J. Phar. 59:173-196, 1990; Holland et al., J. Controlled Release 4:155-0180, 1986).

Representative examples of non-degradable polymers suitable for the delivery of the aforementioned therapeutic agents include poly(ethylene-co-vinyl acetate) (“EVA”) copolymers, aromatic polyesters, such as poly(ethylene terephthalate), silicone rubber, acrylic polymers (polyacrylate, polyacrylic acid, polymethylacrylic acid, polymethylmethacrylate, poly(butyl methacrylate)), poly(alkylcynoacrylate) (e.g., poly(ethylcyanoacrylate), poly(butylcyanoacrylate) poly(hexylcyanoacrylate) poly(octylcyanoacrylate)), acrylic resin, polyethylene, polypropylene, polyamides (nylon 6,6), polyurethanes (e.g., CHRONOFLEX AL and CHRONOFLEX AR (both from CardioTech International, Inc., Wobum, Mass.), TECOFLEX, and BIONATE (Polymer Technology Group, Inc., Emeryville, Calif.)), poly(ester urethanes), poly(ether urethanes), poly(ester-urea), polyethers (poly(ethylene oxide), poly(propylene oxide), polyoxyalkylene ether block copolymers based on ethylene oxide and propylene oxide such as the PLURONIC polymers (e.g., F-127 or F87) from BASF Corporation (Mount Olive, N.J.), and poly(tetramethylene glycol), styrene-based polymers (polystyrene, poly(styrene sulfonic acid), poly(styrene)-block-poly(isobutylene)-block-poly(styrene), poly(styrene)-poly(isoprene) block copolymers), and vinyl polymers (polyvinylpyrrolidone, poly(vinyl alcohol), poly(vinyl acetate phthalate) as well as copolymers and blends thereof. Polymers may also be developed which are either anionic (e.g., alginate, carrageenan, carboxymethyl cellulose, poly(acrylamido-2-methyl propane sulfonic acid) and copolymers thereof, poly(methacrylic acid and copolymers thereof and poly(acrylic acid) and copolymers thereof, as well as blends thereof, or cationic (e.g., chitosan, poly-L-lysine, polyethylenimine, and poly(allyl amine)) and blends thereof (see generally, Dunn et al., J. Applied Polymer Sci. 50:353-365, 1993; Cascone et al., J. Materials Sci.: Materials in Medicine 5:770-774, 1994; Shiraishi et al., Biol. Pharm. Bull. 16(11):1164-1168, 1993; Thacharodi and Rao, Int'l J. Pharm. 120:115-118, 1995; Miyazaki et al., Int'l J. Pharm. 118:257-263, 1995).

Some examples of preferred polymeric carriers for the practice of this invention include poly(ethylene-co-vinyl acetate), polyurethanes, poly (D, L-lactic acid) oligomers and polymers, poly (L-lactic acid) oligomers and polymers, poly (glycolic acid), copolymers of lactic acid and glycolic acid, copolymers of lactide and glycolide, poly (caprolactone), poly (valerolactone), polyanhydrides, copolymers of poly (caprolactone) or poly (lactic acid) with a polyethylene glycol (e.g., MePEG), block copolymers of the form X—Y, X—Y—X, Y—X—Y, R—(Y'X)ₙ, or R—(X—Y)ₙ, where X is a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one and R is a multifunctional initiator), silicone rubbers, poly(styrene)block-poly(isobutylene)-block-poly(styrene), poly(acrylate) polymers and blends, admixtures, or co-polymers of any of the above. Other preferred polymers include collagen, poly(alkylene oxide)-based polymers, polysaccharides such as hyaluronic acid, chitosan and fucans, and copolymers of polysaccharides with degradable polymers.

Other representative polymers capable of sustained localized delivery of anti-infective and/or fibrosis-inhibiting therapeutic agents include carboxylic polymers, polyacetates, polycarbonates, polyethers, polyethylenes, polyvinylbutyrals, polysilanes, polyureas, polyoxides, polystyrenes, polysulfides, polysulfones, polysulfonides, polyvinylhalides, pyrrolidones, rubbers, thermal-setting polymers, cross-linkable acrylic and methacrylic polymers, ethylene acrylic acid copolymers, styrene acrylic copolymers, vinyl acetate polymers and copolymers, vinyl acetal polymers and copolymers, epoxies, melamines, other amino resins, phenolic polymers, and copolymers thereof, water-insoluble cellulose ester polymers (including cellulose acetate propionate, cellulose acetate, cellulose acetate butyrate, cellulose nitrate, cellulose acetate phthalate, and mixtures thereof), polyvinylpyrrolidone, polyethylene glycols, polyethylene oxide, polyvinyl alcohol, polyethers, polysaccharides, hydrophilic polyurethane, polyhydroxyacrylate, dextran, xanthan, hydroxypropyl cellulose, and homopolymers and copolymers of N-vinylpyrrolidone, N-vinyllactam, N-vinyl butyrolactam, N-vinyl caprolactam, other vinyl compounds having polar pendant groups, acrylate and methacrylate having hydrophilic esterifying groups, hydroxyacrylate, and acrylic acid, and combinations thereof; cellulose esters and ethers, ethyl cellulose, hydroxyethyl cellulose, cellulose nitrate, cellulose acetate, cellulose acetate butyrate, cellulose acetate propionate, natural and synthetic elastomers, rubber, acetal, styrene polybutadiene, acrylic resin, polyvinylidene chloride, polycarbonate, homopolymers and copolymers of vinyl compounds, polyvinylchloride, and polyvinylchloride acetate.

Representative examples of patents relating to drug-delivery polymers and their preparation include PCT Publication Nos. WO 98/19713, WO 01/17575, WO 01/41821, WO 01/41822, and WO 01/15526 (as well as the corresponding U.S. applications), U.S. Pat. Nos. 4,500,676, 4,582,865, 4,629,623, 4,636,524, 4,713,448, 4,795,741, 4,913,743, 5,069,899, 5,099,013, 5,128,326, 5,143,724, 5,153,174, 5,246,698, 5,266,563, 5,399,351, 5,525,348, 5,800,412, 5,837,226, 5,942,555, 5,997,517, 6,007,833, 6,071,447, 6,090,995, 6,106,473, 6,110,483, 6,121,027, 6,156,345, 6,214,901, 6,368,611 6,630,155, 6,528,080, RE37,950, 6,46,1631, 6,143,314, 5,990,194, 5,792,469, 5,780,044, 5,759,563, 5,744,153, 5,739,176, 5,733,950, 5,681,873, 5,599,552, 5,340,849, 5,278,202, 5,278,201, 6,589,549, 6,287,588, 6,201,072, 6,117,949, 6,004,573, 5,702,717, 6,413,539, 5,714,159, 5,612,052, and U.S. patent application Publication Nos. 2003/0068377, 2002/0192286, 2002/0076441, and 2002/0090398.

It should be obvious to one of skill in the art that the polymers as described herein can also be blended or copolymerized in various compositions as required to deliver therapeutic doses of biologically active agents.

Polymeric carriers for anti-infective and/or fibrosis-inhibiting therapeutic agents can be fashioned in a variety of forms, with desired release characteristics and/or with specific properties depending upon the composition being utilized. For example, polymeric carriers may be fashioned to release a therapeutic agent upon exposure to a specific triggering event such as pH (see, e.g., Heller et al., “Chemically Self-Regulated Drug Delivery Systems,” in Polymers in Medicine III, Elsevier Science Publishers B. V., Amsterdam, 1988, pp.175-188; Kang et al., J. Applied Polymer Sci. 48:343-354, 1993; Dong et al., J. Controlled Release 19:171-178, 1992; Dong and Hoffman, J. Controlled Release 15:141-152, 1991; Kim et al., J. Controlled Release 28:143-152, 1994; Cornejo-Bravo et al., J. Controlled Release 33:223-229, 1995; Wu and Lee, Pharm. Res. 10(10):1544-1547, 1993; Serres et al., Pharm. Res. 13(2):196-201, 1996; Peppas, “Fundamentals of pH- and Temperature-Sensitive Delivery Systems,” in Gurny et al. (eds.), Pulsatile Drug Delivery, Wissenschaftliche Verlagsgesellschaft mbH, Stuttgart, 1993, pp. 41-55; Doelker, “Cellulose Derivatives,” 1993, in Peppas and Langer (eds.), Biopolymers I, Springer-Verlag, Berlin). Representative examples of pH-sensitive polymers include poly (acrylic acid) and its derivatives (including for example, homopolymers such as poly(aminocarboxylic acid); poly(acrylic acid); poly(methyl acrylic acid), copolymers of such homopolymers, and copolymers of poly(acrylic acid) and/or acrylate or acrylamide Imonomers such as those discussed above. Other pH sensitive polymers include polysaccharides such as cellulose acetate phthalate; hydroxypropylmethylcellulose phthalate; hydroxypropylmethylcellulose acetate succinate; cellulose acetate trimellilate; and chitosan. Yet other pH sensitive polymers include any mixture of a pH sensitive polymer and a water-soluble polymer.

Likewise, ant-infective and/or fibrosis-inhibiting therapeutic agents can be delivered via polymeric carriers which are temperature sensitive (see, e.g., Chen et al., “Novel Hydrogels of a Temperature-Sensitive PLURONIC Grafted to a Bioadhesive Polyacrylic Acid Backbone for Vaginal Drug Delivery,” in Proceed. Intern. Symp. Control. Rel. Bioact. Mater. 22:167-168, Controlled Release Society, Inc., 1995; Okano, “Molecular Design of Stimuli-Responsive Hydrogels for Temporal Controlled Drug Delivery,” in Proceed. Intern. Symp. Control. Rel. Bioact. Mater. 22:111-112, Controlled Release Society, Inc., 1995; Johnston et al., Pharm. Res. 9(3):425-433, 1992; Tung, Int'l J. Pharm. 107:85-90, 1994; Harsh and Gehrke, J. Controlled Release 17:175-186, 1991; Bae et al., Pharm. Res. 8(4):531-537, 1991; Dinarvand and D'Emanuele, J. Controlled Release 36:221-227, 1995; Yu and Grainger, “Novel Thermo-sensitive Amphiphilic Gels: Poly N-isopropylacrylamide-co-sodium acrylate-co-n-N-alkylacrylamide Network Synthesis and Physicochemical Characterization,” Dept. of Chemical & Biological Sci., Oregon Graduate Institute of Science & Technology, Beaverton, Oreg., pp. 820-821; Zhou and Smid, “Physical Hydrogels of Associative Star Polymers,” Polymer Research Institute, Dept. of Chemistry, College of Environmental Science and Forestry, State Univ. of New York, Syracuse, N.Y., pp. 822-823; Hoffman et al., “Characterizing Pore Sizes and Water ‘Structure’ in Stimuli-Responsive Hydrogels,” Center for Bioengineering, Univ. of Washington, Seattle, Wash., p. 828; Yu and Grainger, “Thermo-sensitive Swelling Behavior in Crosslinked N-isopropylacrylamide Networks: Cationic, Anionic and Ampholytic Hydrogels,” Dept. of Chemical & Biological Sci., Oregon Graduate Institute of Science & Technology, Beaverton, Oreg., pp. 829-830; Kim et al., Pharm. Res. 9(3):283-290, 1992; Bae et al., Pharm. Res. 8(5):624-628, 1991; Kono et al., J. Controlled Release 30:69-75, 1994; Yoshida et al., J. Controlled Release 32:97-102, 1994; Okano et al., J. Controlled Release 36:125-133, 1995; Chun and Kim, J. Controlled Release 38:39-47, 1996; D'Emanuele and Dinarvand, Int'l J. Pharm. 118:237-242, 1995; Katono et al., J. Controlled Release 16:215-228, 1991; Hoffman, “Thermally Reversible Hydrogels Containing Biologically Active Species,” in Migliaresi et al. (eds.), Polymers in Medicine III, Elsevier Science Publishers B. V., Amsterdam, 1988, pp.161-167; Hoffman, “Applications of Thermally Reversible Polymers and Hydrogels in Therapeutics and Diagnostics,” in Third International Symposium on Recent Advances in Drug Delivery Systems, Salt Lake City, Utah, Feb. 24-27, 1987, pp. 297-305; Gutowska et al., J. Controlled Release 22:95-104, 1992; Palasis and Gehrke, J. Controlled Release 18:1-12, 1992; Paavola et al., Pharm. Res. 12(12):1997-2002, 1995).

Representative examples of thermogelling polymers, and the gelatin temperature (LCST (° C.)) include homopolymers such as poly(N-methyl-N-n-propylacrylamide), 19.8; poly(N-n-propylacrylamide), 21.5; poly(N-methyl-N-isopropylacrylamide), 22.3; poly(N-n-propylmethacrylamide), 28.0; poly(N-isopropylacrylamide), 30.9; poly(N, n-diethylacrylamide), 32.0; poly(N-isopropylmethacrylamide), 44.0; poly(N-cyclopropylacrylamide), 45.5; poly(N-ethylmethyacrylamide), 50.0; poly(N-methyl-N-ethylacrylamide), 56.0; poly(N-cyclopropylmethacrylamide), 59.0; poly(N-ethylacrylamide), 72.0. Moreover thermogelling polymers may be made by preparing copolymers between (among) monomers of the above, or by combining such homopolymers with other water-soluble polymers such as acrylmonomers (e.g., acrylic acid and derivatives thereof, such as methylacrylic acid, acrylate monomers and derivatives thereof, such as butyl methacrylate, butyl acrylate, lauryl acrylate, and acrylamide monomers and derivatives thereof, such as N-butyl acrylamide and acrylamide).

Other representative examples of thermogelling polymers include cellulose ether derivatives such as hydroxypropyl cellulose, 41° C.; methyl cellulose, 55° C.; hydroxypropylmethyl cellulose, 66° C.; and ethylhydroxyethyl cellulose, polyalkylene oxide-polyester block copolymers of the structure X—Y, Y—X—Y and X—Y—X where X in a polyalkylene oxide and Y is a biodegradable polyester (e.g., PLG-PEG-PLG) and PLURONICs such as F-127, 10-15° C.; L-122, 19° C.; L-92, 26° C.; L-81, 20° C.; and L-61, 24° C.

Representative examples of patents relating to thermally gelling polymers and the preparation include U.S. Pat. Nos. 6,451,346; 6,201,072; 6,117,949; 6,004,573; 5,702,717; and 5,484,610; and PCT Publication Nos. WO 99/07343; WO 99/18142; WO 03/17972; WO 01/82970; WO 00/18821; WO 97/15287; WO 01/41735; WO 00/00222 and WO 00/38651.

Anti-infective and/or fibrosis-inhibiting therapeutic agents may be linked by occlusion in the polymer, dissolution in the polymer, bound by covalent linkages, bound by ionic interactions, or encapsulated in microcapsules. Within certain embodiments of the invention, therapeutic compositions are provided in non-capsular formulations such as microspheres (ranging from nanometers to micrometers in size), pastes, threads of various size, films, or sprays. In one aspect, the anti-scarring agent may be incorporated into biodegradable magnetic nanospheres. The nanospheres may be used, for example, to replenish an anti-scarring agent into an implanted intravascular device, such as a stent containing a weak magnetic alloy (see, e.g., Z. Forbes, B. B. Yellen, G. Friedman, K. Barbee. “An approach to targeted drug delivery based on uniform magnetic fields,” IEEE Trans. Magn. 39(5): 3372-3377 (2003)).

Within certain aspects of the present invention, therapeutic compositions of anti-infective and/or fibrosis-inhibiting agents may be fashioned in the form of microspheres, microparticles and/or nanoparticles having any size ranging from 50 nm to 500 μm, depending upon the particular use. These compositions can be. These compositions can be formed by spray-drying methods, milling methods, coacervation methods, W/O emulsion methods, W/O/W emulsion methods, and solvent evaporation methods. In other aspects, these compositions can include microemulsions, emulsions, liposomes and micelles. Alternatively, such compositions may also be readily applied as a “spray”, which solidifies into a film or coating for use as a device/implant surface coating or to line the tissues of the implantation site. Such sprays may be prepared from microspheres of a wide array of sizes, including for example, from 0.1 μm to 3 μm, from 10 μm to 30 μm, and from 30 μm to 100 μm.

Therapeutic compositions that include anti-infective and/or anti-fibrosis agents may also be prepared in a variety of “paste” or gel forms. For example, within one embodiment of the invention, therapeutic compositions are provided which are liquid at one temperature (e.g., temperature greater than 37° C., such as 40° C., 45° C., 50° C., 55° C. or 60° C.), and solid or semi-solid at another temperature (e.g., ambient body temperature, or any temperature lower than 37° C.). Such “thermopastes” may be readily made utilizing a variety of techniques (see, e.g., PCT Publication WO 98/24427). Other pastes may be applied as a liquid, which solidify in vivo due to dissolution of a water-soluble component of the paste and precipitation of encapsulated drug into the aqueous body environment. These “pastes” and “gels” containing therapeutic agents are particularly useful for application to the surface of tissues that will be in contact with the implant or device.

Within further aspects of the present invention, polymeric carriers are provided which are adapted to contain and release a hydrophobic ant-infective and/or fibrosis-inhibiting compound, and/or the carrier containing the hydrophobic compound in combination with a carbohydrate, protein or polypeptide. Within certain embodiments, the polymeric carrier contains or comprises regions, pockets, or granules of one or more hydrophobic compounds. For example, within one embodiment of the invention, hydrophobic compounds may be incorporated within a matrix which contains the hydrophobic therapeutic compound, followed by incorporation of the matrix within the polymeric carrier. A variety of matrices can be utilized in this regard, including for example, carbohydrates and polysaccharides such as starch, cellulose, dextran, methylcellulose, sodium alginate, heparin, chitosan and hyaluronic acid, proteins or polypeptides such as albumin, collagen and gelatin. Within alternative embodiments, hydrophobic compounds may be contained within a hydrophobic core, and this core contained within a hydrophilic shell.

The anti-infective and/or fibrosis-inhibiting therapeutic agent may be delivered as a solution. The therapeutic agent can be incorporated directly into the solution to provide a homogeneous solution or dispersion. In certain embodiments, the solution is an aqueous solution. The aqueous solution may further include buffer salts, as well as viscosity modifying agents (e.g., hyaluronic acid, alginates, carboxymethylcellulose (CMC), and the like). In another aspect of the invention, the solution can include a biocompatible solvent or liquid oligomers and/or polymers, such as ethanol, DMSO, glycerol, PEG-200, PEG-300 or NMP. These compositions may further comprise a polymer such a degradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, or block copolymers of the form X—Y, Y—X—Y, R—(Y—X)ₙ, R—(X—Y)ₙ and X—Y—X (where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator).

Within another aspect of the invention, the therapeutic anti-infective and/or fibrosis-inhibiting agent can further comprise a secondary carrier. The secondary carrier can be in the form of microspheres (e.g., PLGA, PLLA, PDLLA, PCL, gelatin, polydioxanone, poly(alkylcyanoacrylate)), nanospheres (PLGA, PLLA, PDLLA, PCL, gelatin, polydioxanone, poly(alkylcyanoacrylate)), liposomes, emulsions, microemulsions, micelles (SDS, block copolymers of the form X—Y, Y—X—Y, R—(Y—X)ₙ, R—(X—Y)ₙ and X—Y—X (where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator), zeolites or cyclodextrins.

Other carriers that may likewise be utilized to contain and deliver anti-infective and/or fibrosis-inhibiting therapeutic agents described herein include: hydroxypropyl cyclodextrin (Cserhati and Hollo, Int. J. Pharm. 108:69-75, 1994), liposomes (see, e.g., Sharma et al., Cancer Res. 53:5877-5881, 1993; Sharma and Straubinger, Pharm. Res. 11(60):889-896, 1994; WO 93/18751; U.S. Pat. No. 5,242,073), liposome/gel (WO 94/26254), nanocapsules (Bartoli et al., J. Microencapsulation 7(2):191-197, 1990), micelles (Alkan-Onyuksel et al., Pharm. Res. 11(2):206-212, 1994), implants (Jampel et al., Invest. Ophthalm. Vis. Science 34(11):3076-3083, 1993; Walter et al., Cancer Res. 54:22017-2212, 1994), nanoparticles (Violante and Lanzafame PMCR), nanoparticles—modified (U.S. Pat. No. 5,145,684), nanoparticles (surface modified) (U.S. Pat. No. 5,399,363), micelle (surfactant) (U.S. Pat. No. 5,403,858), synthetic phospholipid compounds (U.S. Pat. No. 4,534,899), gas borne dispersion (U.S. Pat. No. 5,301,664), liquid emulsions, foam, spray, gel, lotion, cream, ointment, dispersed vesicles, particles or droplets solid- or liquid-aerosols, microemulsions (U.S. Pat. No. 5,330,756), polymeric shell (nano- and micro-capsule) (U.S. Pat. No. 5,439,686), emulsion (Tarr et al., Pharm Res. 4: 62-165, 1987), nanospheres (Hagan et al., Proc. Intern. Symp. Control Rel. Bioact. Mater. 22, 1995; Kwon et al., Pharm Res. 12(2):192-195; Kwon et al., Pharm Res. 10(7):970-974; Yokoyama et al., J Contr. Rel. 32:269-277, 1994; Gref et al., Science 263:1600-1603, 1994; Bazile et al., J. Pharm. Sci. 84:493-498, 1994) and implants (U.S. Pat. No. 4,882,168).

Within another aspect of the present invention, polymeric carriers can be materials that are formed in situ. In one embodiment, the precursors can be monomers or macromers that contain unsaturated groups that can be polymerized and/or cross-linked. The monomers or macromers can then, for example, be injected into the treatment area or onto the surface of the treatment area and polymerized in situ using a radiation source (e.g., visible or UV light) or a free radical system (e.g., potassium persulfate and ascorbic acid or iron and hydrogen peroxide). The polymerization step can be performed immediately prior to, simultaneously to or post injection of the reagents into the treatment site. Representative examples of compositions that undergo free radical polymerization reactions are described in WO 01/44307, WO 01/68720, WO 02/072166, WO 03/043552, WO 93/17669, WO 00/64977; U.S. Pat. Nos. 5,900,245, 6,051,248, 6,083,524,6,177,095, 6,201,065, 6,217,894, 6,639,014, 6,352,710, 6,410,645, 6,531,147, 5,567,435, 5,986,043, 6,602,975; U.S. patent application Publication Nos. 2002/012796A1, 2002/0127266A1, 2002/0151650A1, 2003/0104032A1, 2002/0091229A1, and 2003/0059906A1.

In certain aspects, it is desirable to use compositions that can be administered as liquids, but subsequently form hydrogels at the site of administration. Such in situ hydrogel forming compositions can be administered as liquids from a variety of different devices, and are more adaptable for administration to any site, since they are not preformed. Examples of in situ forming hydrogels include photoactivatable mixtures of water-soluble co-polyester prepolymers and polyethylene glycol to create hydrogel barriers. Block copolymers of polyalkylene oxide polymers (e.g., PLURONIC compounds from BASF Corporation, Mount Olive, N.J.) and poloxamers have been designed that are soluble in cold water, but form insoluble hydrogels that adhere to tissues at body temperature (Leach, et al., Am. J. Obstet. Gynecol. 162:1317-1319 (1990)).

As mentioned elsewhere herein, the present invention provides for polymeric crosslinked matrices, and polymeric carriers, that may be used to assist in the prevention of the formation or growth of fibrous connective tissue. The composition may contain and deliver fibrosis-inhibiting agents in the vicinity of the implanted device. The following compositions are particularly useful when it is desired to infiltrate around the device, with or without a fibrosis-inhibiting agent. Such polymeric materials may be prepared from, e.g., (a) synthetic materials, (b) naturally-occurring materials, or (c) mixtures of synthetic and naturally occurring materials. The matrix may be prepared from, e.g., (a) a one-component, i.e., self-reactive, compound, or (b) two or more compounds that are reactive with one another. Typically, these materials are fluid prior to delivery, and thus can be sprayed or otherwise extruded from a delivery device (e.g., a syringe) in order to deliver the composition. After delivery, the component materials react with each other, and/or with the body, to provide the desired affect. In some instances, materials that are reactive with one another must be kept separated prior to delivery to the patient, and are mixed together just prior to being delivered to the patient, in order that they maintain a fluid form prior to delivery. In a preferred aspect of the invention, the components of the matrix are delivered in a liquid state to the desired site in the body, whereupon in situ polymerization occurs.

First and Second Synthetic Polymers

In one embodiment, crosslinked polymer compositions (in other words, crosslinked matrices) are prepared by reacting a first synthetic polymer containing two or more nucleophilic groups with a second synthetic polymer containing two or more electrophilic groups, where the electrophilic groups are capable of covalently binding with the nucleophilic groups. In one embodiment, the first and second polymers are each non-immunogenic. In another embodiment, the matrices are not susceptible to enzymatic cleavage by, e.g., a matrix metalloproteinase (e.g., collagenase) and are therefore expected to have greater long-term persistence in vivo than collagen-based compositions.

As used herein, the term “polymer” refers inter alia to polyalkyls, polyamino acids, polyalkyleneoxides and polysaccharides. Additionally, for external or oral use, the polymer may be polyacrylic acid or carbopol. As used herein, the term “synthetic polymer” refers to polymers that are not naturally occurring and that are produced via chemical synthesis. As such, naturally occurring proteins such as collagen and naturally occurring polysaccharides such as hyaluronic acid are specifically excluded. Synthetic collagen, and synthetic hyaluronic acid, and their derivatives, are included. Synthetic polymers containing either nucleophilic or electrophilic groups are also referred to herein as “multifunctionally activated synthetic polymers.” The term “multifunctionally activated” (or, simply, “activated”) refers to synthetic polymers which have, or have been chemically modified to have, two or more nucleophilic or electrophilic groups which are capable of reacting with one another (i.e., the nucleophilic groups react with the electrophilic groups) to form covalent bonds. Types of multifunctionally activated synthetic polymers include difunctionally activated, tetrafunctionally activated, and star-branched polymers.

Multifunctionally activated synthetic polymers for use in the present invention must contain at least two, more preferably, at least three, functional groups in order to form a three-dimensional crosslinked network with synthetic polymers containing multiple nucleophilic groups (i.e., “multi-nucleophilic polymers”). In other words, they must be at least difunctionally activated, and are more preferably trifunctionally or tetrafunctionally activated. If the first synthetic polymer is a difunctionally activated synthetic polymer, the second synthetic polymer must contain three or more functional groups in order to obtain a three-dimensional crosslinked network. Most preferably, both the first and the second synthetic polymer contain at least three functional groups.

Synthetic polymers containing multiple nucleophilic groups are also referred to generically herein as “multi-nucleophilic polymers.” For use in the present invention, multi-nucleophilic polymers must contain at least two, more preferably, at least three, nucleophilic groups. If a synthetic polymer containing only two nucleophilic groups is used, a synthetic polymer containing three or more electrophilic groups must be used in order to obtain a three-dimensional crosslinked network.

Preferred multi-nucleophilic polymers for use in the compositions and methods of the present invention include synthetic polymers that contain, or have been modified to contain, multiple nucleophilic groups such as primary amino groups and thiol groups. Preferred multi-nucleophilic polymers include: (i) synthetic polypeptides that have been synthesized to contain two or more primary amino groups or thiol groups; and (ii) polyethylene glycols that have been modified to contain two or more primary amino groups or thiol groups. In general, reaction of a thiol group with an electrophilic group tends to proceed more slowly than reaction of a primary amino group with an electrophilic group.

In one embodiment, the multi-nucleophilic polypeptide is a synthetic polypeptide that has been synthesized to incorporate amino acid residues containing primary amino groups (such as lysine) and/or amino acids containing thiol groups (such as cysteine). Poly(lysine), a synthetically produced polymer of the amino acid lysine (145 MW), is particularly preferred. Poly(lysine)s have been prepared having anywhere from 6 to about 4,000 primary amino groups, corresponding to molecular weights of about 870 to about 580,000.

Poly(lysine)s for use in the present invention preferably have a molecular weight within the range of about 1,000 to about 300,000; more preferably, within the range of about 5,000 to about 100,000; most preferably, within the range of about 8,000 to about 15,000. Poly(lysine)s of varying molecular weights are commercially available from Peninsula Laboratories, Inc. (Belmont, Calif.) and Aldrich Chemical (Milwaukee, Wis.).

Polyethylene glycol can be chemically modified to contain multiple primary amino or thiol groups according to methods set forth, for example, in Chapter 22 of Poly(ethylene Glycol) Chemistry: Biotechnical and Biomedical Applications, J. Milton Harris, ed., Plenum Press, N.Y. (1992). Polyethylene glycols which have been modified to contain two or more primary amino groups are referred to herein as “multi-amino PEGs.” Polyethylene glycols which have been modified to contain two or more thiol groups are referred to herein as “multi-thiol PEGs.” As used herein, the term “polyethylene glycol(s)” includes modified and or derivatized polyethylene glycol(s).

Various forms of multi-amino PEG are commercially available from Shearwater Polymers (Huntsville, Ala.) and from Huntsman Chemical Company (Utah) under the name “Jeffamine.” Multi-amino PEGs useful in the present invention include Huntsman's Jeffamine diamines (“D” series) and triamines (“T” series), which contain two and three primary amino groups per molecule, respectively.

Polyamines such as ethylenediamine (H₂N—CH₂—CH₂—NH₂), tetramethylenediamine (H₂N—(CH₂)₄—NH₂), pentamethylenediamine (cadaverine) (H₂N—(CH₂)₅—NH₂), hexamethylenediamine (H₂N—(CH₂)₆—NH₂), di(2-aminoethyl)amine (HN—(CH₂—CH₂—NH₂)₂), and tris(2-aminoethyl)amine (N—(CH₂—CH₂—NH₂)₃) may also be used as the synthetic polymer containing multiple nucleophilic groups.

Synthetic polymers containing multiple electrophilic groups are also referred to herein as “multi-electrophilic polymers.” For use in the present invention, the multifunctionally activated synthetic polymers must contain at least two, more preferably, at least three, electrophilic groups in order to form a three-dimensional crosslinked network with multi-nucleophilic polymers. Preferred multi-electrophilic polymers for use in the compositions of the invention are polymers which contain two or more succinimidyl groups capable of forming covalent bonds with nucleophilic groups on other molecules. Succinimidyl groups are highly reactive with materials containing primary amino (NH₂) groups, such as multi-amino PEG, poly(lysine), or collagen. Succinimidyl groups are slightly less reactive with materials containing thiol (SH) groups, such as multi-thiol PEG or synthetic polypeptides containing multiple cysteine residues.

As used herein, the term “containing two or more succinimidyl groups” is meant to encompass polymers which are preferably commercially available containing two or more succinimidyl groups, as well as those that must be chemically derivatized to contain two or more succinimidyl groups. As used herein, the term “succinimidyl group” is intended to encompass sulfosuccinimidyl groups and other such variations of the “generic” succinimidyl group. The presence of the sodium sulfite moiety on the sulfosuccinimidyl group serves to increase the solubility of the polymer.

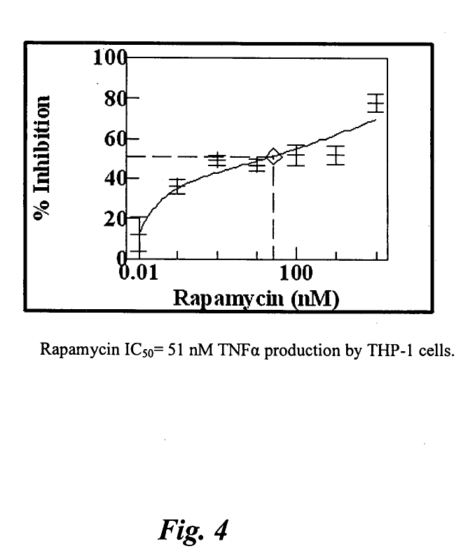

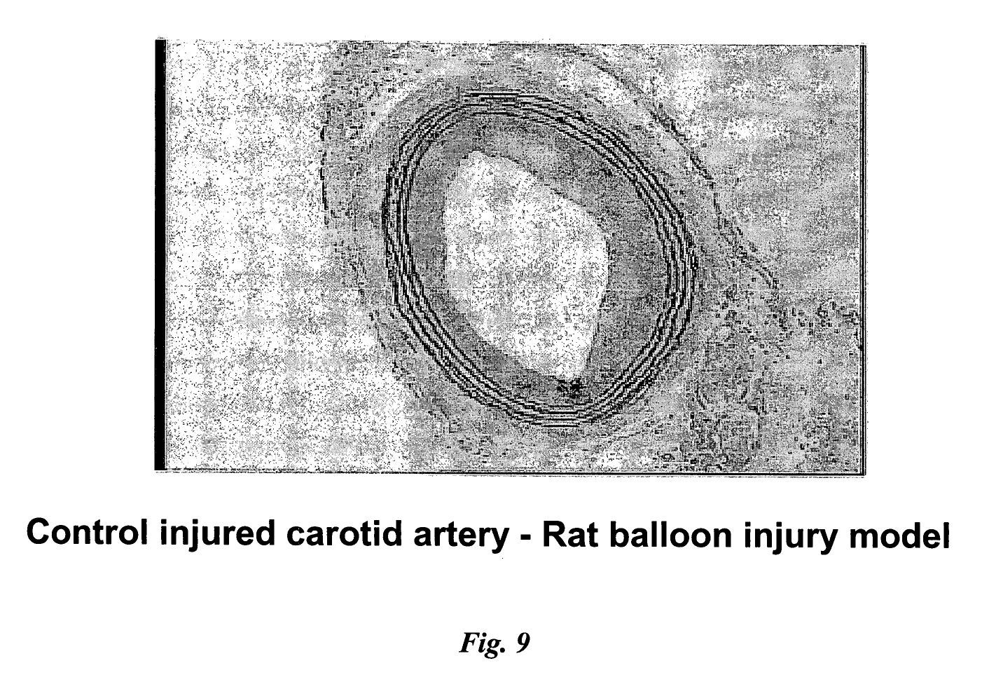

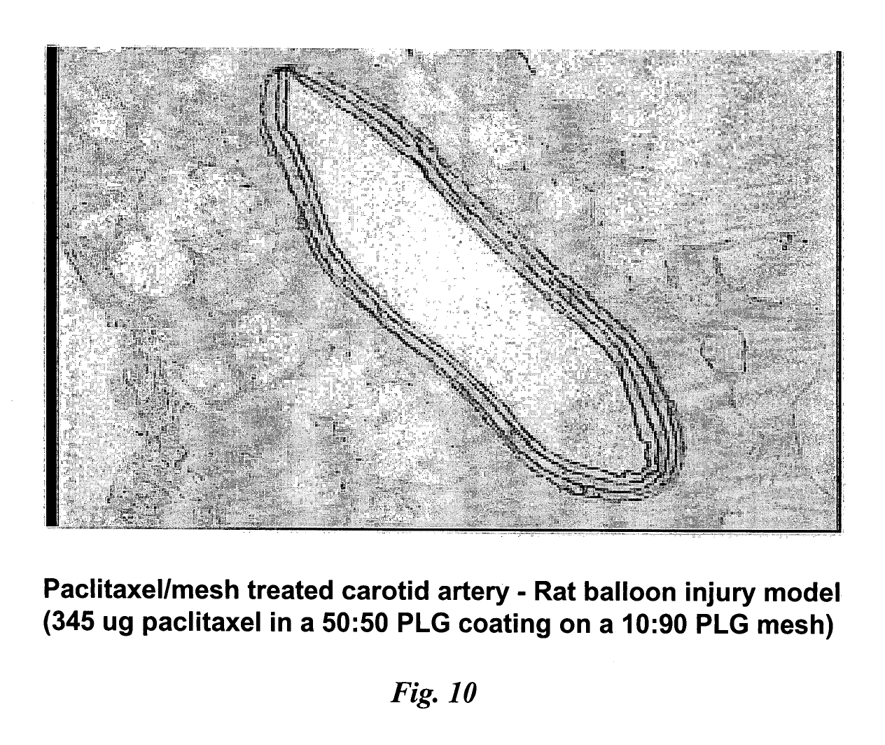

Hydrophilic polymers and, in particular, various derivatized polyethylene glycols, are preferred for use in the compositions of the present invention. As used herein, the term “PEG” refers to polymers having the repeating structure (OCH₂—CH₂)ₙ. Structures for some specific, tetrafunctionally activated forms of PEG are shown in FIGS. 4 to 13 of U.S. Pat. 5,874,500, incorporated herein by reference. Examples of suitable PEGS include PEG succinimidyl propionate (SE-PEG), PEG succinimidyl succinamide (SSA-PEG), and PEG succinimidyl carbonate (SC-PEG). In one aspect of the invention, the crosslinked matrix is formed in situ by reacting pentaerythritol poly(ethylene glycol)ether tetra-sulfhydryl](4-armed thiol PEG) and pentaerythritol poly(ethylene glycol)ether tetra-succinimidyl glutarate](4-armed NHS PEG) as reactive reagents. Structures for these reactants are shown in U.S. Pat. No. 5,874,500. Each of these materials has a core with a structure that may be seen by adding ethylene oxide-derived residues to each of the hydroxyl groups in pentaerythritol, and then derivatizing the terminal hydroxyl groups (derived from the ethylene oxide) to contain either thiol groups (so as to form 4-armed thiol PEG) or N-hydroxysuccinimydyl groups (so as to form 4-armed NHS PEG), optionally with a linker group present between the ethylene oxide derived backbone and the reactive functional group, where this product is commercially available as COSEAL from Angiotech Pharmaceuticals Inc. Optionally, a group “D” may be present in one or both of these molecules, as discussed in more detail below.

As discussed above, preferred activated polyethylene glycol derivatives for use in the invention contain succinimidyl groups as the reactive group. However, different activating groups can be attached at sites along the length of the PEG molecule. For example, PEG can be derivatized to form functionally activated PEG propionaldehyde (A-PEG), or functionally activated PEG glycidyl ether (E-PEG), or functionally activated PEG-isocyanate (I-PEG), or functionally activated PEG-vinylsulfone (V-PEG).

Hydrophobic polymers can also be used to prepare the compositions of the present invention. Hydrophobic polymers for use in the present invention preferably contain, or can be derivatized to contain, two or more electrophilic groups, such as succinimidyl groups, most preferably, two, three, or four electrophilic groups. As used herein, the term “hydrophobic polymer” refers to polymers which contain a relatively small proportion of oxygen or nitrogen atoms.

Hydrophobic polymers which already contain two or more succinimidyl groups include, without limitation, disuccinimidyl suberate (DSS), bis(sulfosuccinimidyl) suberate (BS3), dithiobis(succinimidylpropionate) (DSP), bis(2-succinimidooxycarbonyloxy) ethyl sulfone (BSOCOES), and 3,3′-dithiobis(sulfosuccinimidylpropionate (DTSPP), and their analogs and derivatives. The above-referenced polymers are commercially available from Pierce (Rockford, III.), under catalog Nos. 21555, 21579, 22585, 21554, and 21577, respectively.

Preferred hydrophobic polymers for use in the invention generally have a carbon chain that is no longer than about 14 carbons. Polymers having carbon chains substantially longer than 14 carbons generally have very poor solubility in aqueous solutions and, as such, have very long reaction times when mixed with aqueous solutions of synthetic polymers containing multiple nucleophilic groups.

Certain polymers, such as polyacids, can be derivatized to contain two or more functional groups, such as succinimidyl groups. Polyacids for use in the present invention include, without limitation, trimethylolpropane-based tricarboxylic acid, di(trimethylol propane)-based tetracarboxylic acid, heptanedioic acid, octanedioic acid (suberic acid), and hexadecanedioic acid (thapsic acid). Many of these polyacids are commercially available from DuPont Chemical Company (Wilmington, Del.). According to a general method, polyacids can be chemically derivatized to contain two or more succinimidyl groups by reaction with an appropriate molar amount of N-hydroxysuccinimide (NHS) in the presence of N,N′-dicyclohexylcarbodiimide (DCC).

Polyalcohols such as trimethylolpropane and di(trimethylol propane) can be converted to carboxylic acid form using various methods, then further derivatized by reaction with NHS in the presence of DCC to produce trifunctionally and tetrafunctionally activated polymers, respectively, as described in U.S. application Ser. No. 08/403,358. Polyacids such as heptanedioic acid (HOOC—(CH₂)₅—COOH), octanedioic acid (HOOC—(CH₂)₆—COOH), and hexadecanedioic acid (HOOC—(CH₂)₁₄—COOH) are derivatized by the addition of succinimidyl groups to produce difunctionally activated polymers.

Polyamines such as ethylenediamine, tetramethylenediamine, pentamethylenediamine (cadaverine), hexamethylenediamine, bis (2-aminoethyl)amine, and tris(2-aminoethyl)amine can be chemically derivatized to polyacids, which can then be derivatized to contain two or more succinimidyl groups by reacting with the appropriate molar amounts of N-hydroxysuccinimide in the presence of DCC, as described in U.S. application Ser. No. 08/403,358. Many of these polyamines are commercially available from DuPont Chemical Company.

In a preferred embodiment, the first synthetic polymer will contain multiple nucleophilic groups (represented below as “X”) and it will react with the second synthetic polymer containing multiple electrophilic groups (represented below as “Y”), resulting in a covalently bound polymer network, as follows:
Polymer-Xₘ+Polymer-Yₙ→Polymer-Z-Polymer
wherein m≦2, n≦2, and m+n≦5;
where exemplary X groups include —NH₂, —SH, —OH, —PH₂, CO—NH—NH₂, etc., where the X groups may be the same or different in polymer-Xₘ;
where exemplary Y groups include —CO₂—N(COCH₂)₂, —CO₂H, —CHO, —CHOCH₂ (epoxide), —N═C═O, —SO₂—CH═CH₂, —N(COCH)₂ (i.e., a five-membered heterocyclic ring with a double bond present between the two CH groups), —S—S—(C₅H₄N), etc., where the Y groups may be the same or different in polymer-Yₙ; and
where Z is the functional group resulting from the union of a nucleophilic group (X) and an electrophilic group (Y).

As noted above, it is also contemplated by the present invention that X and Y may be the same or different, i.e., a synthetic polymer may have two different electrophilic groups, or two different nucleophilic groups, such as with glutathione.

In one embodiment, the backbone of at least one of the synthetic polymers comprises alkylene oxide residues, e.g., residues from ethylene oxide, propylene oxide, and mixtures thereof. The term ‘backbone’ refers to a significant portion of the polymer.

For example, the synthetic polymer containing alkylene oxide residues may be described by the formula X-polymer-X or Y-polymer-Y, wherein X and Y are as defined above, and the term “polymer” represents —(CH₂CH₂O)ₙ— or —(CH(CH₃)CH₂O)ₙ— or —(CH₂—CH₂—O)ₙ—(CH(CH₃)CH₂—O)ₙ—. In these cases the synthetic polymer would be difunctional.

The required functional group X or Y is commonly coupled to the polymer backbone by a linking group (represented below as “Q”), many of which are known or possible. There are many ways to prepare the various functionalized polymers, some of which are listed below:
Polymer-Q₁-X+Polymer-Q₂-Y→Polymer-Q₁-Z-Q₂-Polymer

Exemplary Q groups include —O—(CH₂)ₙ—; —S—(CH₂)ₙ—; —NH—(CH₂)ₙ—; —O₂C—NH—(CH₂)ₙ—; —O₂C—(CH₂)ₙ—; —O₂C—(CR¹H)ₙ—; and —O—R₂—CO—NH—, which provide synthetic polymers of the partial structures: polymer-O—(CH₂)ₙ—(X or Y); polymer-S—(CH₂)ₙ—(X or Y); polymer-NH—(CH₂)ₙ—(X or Y); polymer-O₂C—NH—(CH₂)ₙ—(X or Y); polymer-O₂C—(CH₂)ₙ—(X or Y); polymer-O₂C—(CR¹H)ₙ—(X or Y); and polymer-O—R₂—CO—NH—(X or Y), respectively. In these structures, n=1-10, R¹═H or alkyl (i.e., CH₃, C₂H₅, etc.); R²═CH₂, or CO—NH—CH₂CH₂; and Q₁ and Q₂ may be the same or different.

For example, when Q₂=OCH₂CH₂ (there is no Q₁ in this case); Y═—CO₂—N(COCH₂)₂; and X═—NH₂, —SH, or —OH, the resulting reactions and Z groups would be as follows:
Polymer-NH₂+Polymer-O—-CH₂—CH₂—CO₂—N(COCH₂)₂→Polymer-NH—CO—CH₂—CH₂—O-Polymer;
Polymer-SH+Polymer-O—CH₂—CH₂—CO₂—N(COCH₂)₂→Polymer-S—COCH₂CH₂—O-Polymer; and
Polymer-OH+Polymer-O—CH₂—CH₂—CO₂—N(COCH₂)₂→Polymer-O—COCH₂CH₂—O-Polymer.

An additional group, represented below as “D”, can be inserted between the polymer and the linking group, if present. One purpose of such a D group is to affect the degradation rate of the crosslinked polymer composition in vivo, for example, to increase the degradation rate, or to decrease the degradation rate. This may be useful in many instances, for example, when drug has been incorporated into the matrix, and it is desired to increase or decrease polymer degradation rate so as to influence a drug delivery profile in the desired direction. An illustration of a crosslinking reaction involving first and second synthetic polymers each having D and Q groups is shown below.
Polymer-D-Q-X+Polymer-D-Q-Y→Polymer-D-Q-Z-Q-D-Polymer

Some useful biodegradable groups “D” include polymers formed from one or more α-hydroxy acids, e.g., lactic acid, glycolic acid, and the cyclization products thereof (e.g., lactide, glycolide), ε-caprolactone, and amino acids. The polymers may be referred to as polylactide, polyglycolide, poly(co-lactide-glycolide); poly-ε-caprolactone, polypeptide (also known as poly amino acid, for example, various di- or tri-peptides) and poly(anhydride)s.

In a general method for preparing the crosslinked polymer compositions used in the context of the present invention, a first synthetic polymer containing multiple nucleophilic groups is mixed with a second synthetic polymer containing multiple electrophilic groups. Formation of a three-dimensional crosslinked network occurs as a result of the reaction between the nucleophilic groups on the first synthetic polymer and the electrophilic groups on the second synthetic polymer.

The concentrations of the first synthetic polymer and the second synthetic polymer used to prepare the compositions of the present invention will vary depending upon a number of factors, including the types and molecular weights of the particular synthetic polymers used and the desired end use application. In general, when using multi-amino PEG as the first synthetic polymer, it is preferably used at a concentration in the range of about 0.5 to about 20 percent by weight of the final composition, while the second synthetic polymer is used at a concentration in the range of about 0.5 to about 20 percent by weight of the final composition. For example, a final composition having a total weight of 1 gram (1000 milligrams) would contain between about 5 to about 200 milligrams of multi-amino PEG, and between about 5 to about 200 milligrams of the second synthetic polymer.

Use of higher concentrations of both first and second synthetic polymers will result in the formation of a more tightly crosslinked network, producing a stiffer, more robust gel. Compositions intended for use in tissue augmentation will generally employ concentrations of first and second synthetic polymer that fall toward the higher end of the preferred concentration range. Compositions intended for use as bioadhesives or in adhesion prevention do not need to be as firm and may therefore contain lower polymer concentrations.

Because polymers containing multiple electrophilic groups will also react with water, the second synthetic polymer is generally stored and used in sterile, dry form to prevent the loss of crosslinking ability due to hydrolysis which typically occurs upon exposure of such electrophilic groups to aqueous media. Processes for preparing synthetic hydrophilic polymers containing multiple electrophylic groups in sterile, dry form are set forth in U.S. Pat. No. 5,643,464. For example, the dry synthetic polymer may be compression molded into a thin sheet or membrane, which can then be sterilized using gamma or, preferably, e-beam irradiation. The resulting dry membrane or sheet can be cut to the desired size or chopped into smaller size particulates. In contrast, polymers containing multiple nucleophilic groups are generally not water-reactive and can therefore be stored in aqueous solution.

In certain embodiments, one or both of the electrophilic- or nucleophilic-terminated polymers described above can be combined with a synthetic or naturally occurring polymer. The presence of the synthetic or naturally occurring polymer may enhance the mechanical and/or adhesive properties of the in situ forming compositions. Naturally occurring polymers, and polymers derived from naturally occurring polymer that may be included in in situ forming materials include naturally occurring proteins, such as collagen, collagen derivatives (such as methylated collagen), fibrinogen, thrombin, albumin, fibrin, and derivatives of and naturally occurring polysaccharides, such as glycosaminoglycans, including deacetylated and desulfated glycosaminoglycan derivatives.

In one aspect, a composition comprising naturally-occurring protein and both of the first and second synthetic polymer as described above is used to form the crosslinked matrix according to the present invention. In one aspect, a composition comprising collagen and both of the first and second synthetic polymer as described above is used to form the crosslinked matrix according to the present invention. In one aspect, a composition comprising methylated collagen and both of the first and second synthetic polymer as described above is used to form the crosslinked matrix according to the present invention. In one aspect, a composition comprising fibrinogen and both of the first and second synthetic polymer as described above is used to form the crosslinked matrix according to the present invention. In one aspect, a composition comprising thrombin and both of the first and second synthetic polymer as described above is used to form the crosslinked matrix according to the present invention. In one aspect, a composition comprising albumin and both of the first and second synthetic polymer as described above is used to form the crosslinked matrix according to the present invention. In one aspect, a composition comprising fibrin and both of the first and second synthetic polymer as described above is used to form the crosslinked matrix according to the present invention. In one aspect, a composition comprising naturally occurring polysaccharide and both of the first and second synthetic polymer as described above is used to form the crosslinked matrix according to the present invention. In one aspect, a composition comprising glycosaminoglycan and both of the first and second synthetic polymer as described above is used to form the crosslinked matrix according to the present invention. In one aspect, a composition comprising deacetylated glycosaminoglycan and both of the first and second synthetic polymer as described above is used to form the crosslinked matrix according to the present invention. In one aspect, a composition comprising desulfated glycosaminoglycan and both of the first and second synthetic polymer as described above is used to form the crosslinked matrix according to the present invention.

In one aspect, a composition comprising naturally-occurring protein and the first synthetic polymer as described above is used to form the crosslinked matrix according to the present invention. In one aspect, a composition comprising collagen and the first synthetic polymer as described above is used to form the crosslinked matrix according to the present invention. In one aspect, a composition comprising methylated collagen and the first synthetic polymer as described above is used to form the crosslinked matrix according to the present invention. In one aspect, a composition comprising fibrinogen and the first synthetic polymer as described above is used to form the crosslinked matrix according to the present invention. In one aspect, a composition comprising thrombin and the first synthetic polymer as described above is used to form the crosslinked matrix according to the present invention. In one aspect, a composition comprising albumin and the first synthetic polymer as described above is used to form the crosslinked matrix according to the present invention. In one aspect, a composition comprising fibrin and the first synthetic polymer as described above is used to form the crosslinked matrix according to the present invention. In one aspect, a composition comprising naturally occurring polysaccharide and the first synthetic polymer as described above is used to form the crosslinked matrix according to the present invention. In one aspect, a composition comprising glycosaminoglycan and the first synthetic polymer as described above is used to form the crosslinked matrix according to the present invention. In one aspect, a composition comprising deacetylated glycosaminoglycan and the first synthetic polymer as described above is used to form the crosslinked matrix according to the present invention. In one aspect, a composition comprising desulfated glycosaminoglycan and the first synthetic polymer as described above is used to form the crosslinked matrix according to the present invention.

In one aspect, a composition comprising naturally-occurring protein and the second synthetic polymer as described above is used to form the crosslinked matrix according to the present invention. In one aspect, a composition comprising collagen and the second synthetic polymer as described above is used to form the crosslinked matrix according to the present invention. In one aspect, a composition comprising methylated collagen and the second synthetic polymer as described above is used to form the crosslinked matrix according to the present invention. In one aspect, a composition comprising fibrinogen and the second synthetic polymer as described above is used to form the crosslinked matrix according to the present invention. In one aspect, a composition comprising thrombin and the second synthetic polymer as described above is used to form the crosslinked matrix according to the present invention. In one aspect, a composition comprising albumin and the second synthetic polymer as described above is used to form the crosslinked matrix according to the present invention. In one aspect, a composition comprising fibrin and the second synthetic polymer as described above is used to form the crosslinked matrix according to the present invention. In one aspect, a composition comprising naturally occurring polysaccharide and the second synthetic polymer as described above is used to form the crosslinked matrix according to the present invention. In one aspect, a composition comprising glycosaminoglycan and the second synthetic polymer as described above is used to form the crosslinked matrix according to the present invention. In one aspect, a composition comprising deacetylated glycosaminoglycan and the second synthetic polymer as described above is used to form the crosslinked matrix according to the present invention. In one aspect, a composition comprising desulfated glycosaminoglycan and the second synthetic polymer as described above is used to form the crosslinked matrix according to the present invention.

The presence of protein or polysaccharide components which contain functional groups that can react with the functional groups on multiple activated synthetic polymers can result in formation of a crosslinked synthetic polymer-naturally occurring polymer matrix upon mixing and/or crosslinking of the synthetic polymer(s). In particular, when the naturally occurring polymer (protein or polysaccharide) also contains nucleophilic groups such as primary amino groups, the electrophilic groups on the second synthetic polymer will react with the primary amino groups on these components, as well as the nucleophilic groups on the first synthetic polymer, to cause these other components to become part of the polymer matrix. For example, lysine-rich proteins such as collagen may be especially reactive with electrophilic groups on synthetic polymers.

In one aspect, the naturally occurring protein is polymer may be collagen. As used herein, the term “collagen” or “collagen material” refers to all forms of collagen, including those which have been processed or otherwise modified and is intended to encompass collagen of any type, from any source, including, but not limited to, collagen extracted from tissue or produced recombinantly, collagen analogues, collagen derivatives, modified collagens, and denatured collagens, such as gelatin.

In general, collagen from any source may be included in the compositions of the invention; for example, collagen may be extracted and purified from human or other mammalian source, such as bovine or porcine corium and human placenta, or may be recombinantly or otherwise produced. The preparation of purified, substantially non-antigenic collagen in solution from bovine skin is well known in the art. U.S. Pat. No. 5,428,022 discloses methods of extracting and purifying collagen from the human placenta. U.S. Pat. No. 5,667,839, discloses methods of producing recombinant human collagen in the milk of transgenic animals, including transgenic cows. Collagen of any type, including, but not limited to, types I, II, III, IV, or any combination thereof, may be used in the compositions of the invention, although type I is generally preferred. Either atelopeptide or telopeptide-containing collagen may be used; however, when collagen from a xenogeneic source, such as bovine collagen, is used, atelopeptide collagen is generally preferred, because of its reduced immunogenicity compared to telopeptide-containing collagen.

Collagen that has not been previously crosslinked by methods such as heat, irradiation, or chemical crosslinking agents is preferred for use in the compositions of the invention, although previously crosslinked collagen may be used. Non-crosslinked atelopeptide fibrillar collagen is commercially available from Inamed Aesthetics (Santa Barbara, Calif.) at collagen concentrations of 35 mg/ml and 65 mg/ml under the trademarks ZYDERM I Collagen and ZYDERM II Collagen, respectively. Glutaraldehyde crosslinked atelopeptide fibrillar collagen is commercially available from Inamed Corporation (Santa Barbara, Calif.) at a collagen concentration of 35 mg/ml under the trademark ZYPLAST Collagen.

Collagens for use in the present invention are generally in aqueous suspension at a concentration between about 20 mg/ml to about 120 mg/ml; preferably, between about 30 mg/ml to about 90 mg/ml.

Because of its tacky consistency, nonfibrillar collagen may be preferred for use in compositions that are intended for use as bioadhesives. The term “nonfibrillar collagen” refers to any modified or unmodified collagen material that is in substantially nonfibrillar form at pH 7, as indicated by optical clarity of an aqueous suspension of the collagen.

Collagen that is already in nonfibrillar form may be used in the compositions of the invention. As used herein, the term “nonfibrillar collagen” is intended to encompass collagen types that are nonfibrillar in native form, as well as collagens that have been chemically modified such that they are in nonfibrillar form at or around neutral pH. Collagen types that are nonfibrillar (or microfibrillar) in native form include types IV, VI, and VII.

Chemically modified collagens that are in nonfibrillar form at neutral pH include succinylated collagen and methylated collagen, both of which can be prepared according to the methods described in U.S. Pat. No. 4,164,559, issued Aug. 14, 1979, to Miyata et al., which is hereby incorporated by reference in its entirety. Due to its inherent tackiness, methylated collagen is particularly preferred for use in bioadhesive compositions, as disclosed in U.S. application Ser. No. 08/476,825.

Collagens for use in the crosslinked polymer compositions of the present invention may start out in fibrillar form, then be rendered nonfibrillar by the addition of one or more fiber disassembly agent. The fiber disassembly agent must be present in an amount sufficient to render the collagen substantially nonfibrillar at pH 7, as described above. Fiber disassembly agents for use in the present invention include, without limitation, various biocompatible alcohols, amino acids (e.g., arginine), inorganic salts (e.g., sodium chloride and potassium chloride), and carbohydrates (e.g., various sugars including sucrose).

In one aspect, the polymer may be collagen or a collagen derivative, for example methylated collagen. An example of an in situ forming composition uses pentaerythritol poly(ethylene glycol)ether tetra-sulfhydryl](4-armed thiol PEG), pentaerythritol poly(ethylene glycol)ether tetra-succinimidyl glutarate](4-armed NHS PEG) and methylated collagen as the reactive reagents. This composition, when mixed with the appropriate buffers can produce a crosslinked hydrogel. (See, e.g., U.S. Pat. Nos. 5,874,500; 6,051,648; 6,166,130; 5,565,519 and 6,312,725).

In another aspect, the naturally occurring polymer may be a glycosaminoglycan. Glycosaminoglycans, e.g., hyaluronic acid, contain both anionic and cationic functional groups along each polymeric chain, which can form intramolecular and/or intermolecular ionic crosslinks, and are responsible for the thixotropic (or shear thinning) nature of hyaluronic acid.

In certain aspects, the glycosaminoglycan may be derivatized. For example, glycosaminoglycans can be chemically derivatized by, e.g., deacetylation, desulfation, or both in order to contain primary amino groups available for reaction with electrophilic groups on synthetic polymer molecules. Glycosaminoglycans that can be derivatized according to either or both of the aforementioned methods include the following: hyaluronic acid, chondroitin sulfate A, chondroitin sulfate B (dermatan sulfate), chondroitin sulfate C, chitin (can be derivatized to chitosan), keratan sulfate, keratosulfate, and heparin. Derivatization of glycosaminoglycans by deacetylation and/or desulfation and covalent binding of the resulting glycosaminoglycan derivatives with synthetic hydrophilic polymers is described in further detail in commonly assigned, allowed U.S. patent application Ser. No. 08/146,843, filed Nov. 3, 1993.

In general, the collagen is added to the first synthetic polymer, then the collagen and first synthetic polymer are mixed thoroughly to achieve a homogeneous composition. The second synthetic polymer is then added and mixed into the collagen/first synthetic polymer mixture, where it will covalently bind to primary amino groups or thiol groups on the first synthetic polymer and primary amino groups on the collagen, resulting in the formation of a homogeneous crosslinked network. Various deacetylated and/or desulfated glycosaminoglycan derivatives can be incorporated into the composition in a similar manner as that described above for collagen. In addition, the introduction of hydrocolloids such as carboxymethylcellulose may promote tissue adhesion and/or swellability.

Administration of the Crosslinked Synthetic Polymer Compositions

The compositions of the present invention having two synthetic polymers may be administered before, during or after crosslinking of the first and second synthetic polymer. Certain uses, which are discussed in greater detail below, such as tissue augmentation, may require the compositions to be crosslinked before administration, whereas other applications, such as tissue adhesion, require the compositions to be administered before crosslinking has reached “equilibrium.” The point at which crosslinking has reached equilibrium is defined herein as the point at which the composition no longer feels tacky or sticky to the touch.

In order to administer the composition prior to crosslinking, the first synthetic polymer and second synthetic polymer may be contained within separate barrels of a dual-compartment syringe. In this case, the two synthetic polymers do not actually mix until the point at which the two polymers are extruded from the tip of the syringe needle into the patient's tissue. This allows the vast majority of the crosslinking reaction to occur in situ, avoiding the problem of needle blockage which commonly occurs if the two synthetic polymers are mixed too early and crosslinking between the two components is already too advanced prior to delivery from the syringe needle. The use of a dual-compartment syringe, as described above, allows for the use of smaller diameter needles, which is advantageous when performing soft tissue augmentation in delicate facial tissue, such as that surrounding the eyes.

Alternatively, the first synthetic polymer and second synthetic polymer may be mixed according to the methods described above prior to delivery to the tissue site, then injected to the desired tissue site immediately (preferably, within about 60 seconds) following mixing.

In another embodiment of the invention, the first synthetic polymer and second synthetic polymer are mixed, then extruded and allowed to crosslink into a sheet or other solid form. The crosslinked solid is then dehydrated to remove substantially all unbound water. The resulting dried solid may be ground or comminuted into particulates, then suspended in a nonaqueous fluid carrier, including, without limitation, hyaluronic acid, dextran sulfate, dextran, succinylated noncrosslinked collagen, methylated noncrosslinked collagen, glycogen, glycerol, dextrose, maltose, triglycerides of fatty acids (such as corn oil, soybean oil, and sesame oil), and egg yolk phospholipid. The suspension of particulates can be injected through a small-gauge needle to a tissue site. Once inside the tissue, the crosslinked polymer particulates will rehydrate and swell in size at least five-fold.

Hydrophilic Polymer+Plurality of Crosslinkable Components

As mentioned above, the first and/or second synthetic polymers may be combined with a hydrophilic polymer, e.g., collagen or methylated collagen, to form a composition useful in the present invention. In one general embodiment, the compositions useful in the present invention include a hydrophilic polymer in combination with two or more crosslinkable components. This embodiment is described in further detail in this section.

The Hydrophilic Polymer Component:

The hydrophilic polymer component may be a synthetic or naturally occurring hydrophilic polymer. Naturally occurring hydrophilic polymers include, but are not limited to: proteins such as collagen and derivatives thereof, fibronectin, albumins, globulins, fibrinogen, and fibrin, with collagen particularly preferred; carboxylated polysaccharides such as polymannuronic acid and polygalacturonic acid; aminated polysaccharides, particularly the glycosaminoglycans, e.g., hyaluronic acid, chitin, chondroitin sulfate A, B, or C, keratin sulfate, keratosulfate and heparin; and activated polysaccharides such as dextran and starch derivatives. Collagen (e.g., methylated collagen) and glycosaminoglycans are preferred naturally occurring hydrophilic polymers for use herein.

In general, collagen from any source may be used in the composition of the method; for example, collagen may be extracted and purified from human or other mammalian source, such as bovine or porcine corium and human placenta, or may be recombinantly or otherwise produced. The preparation of purified, substantially non-antigenic collagen in solution from bovine skin is well known in the art. See, e.g., U.S. Pat. No. 5,428,022, to Palefsky et al., which discloses methods of extracting and purifying collagen from the human placenta. See also U.S. Pat. No. 5,667,839, to Berg, which discloses methods of producing recombinant human collagen in the milk of transgenic animals, including transgenic cows. Unless otherwise specified, the term “collagen” or “collagen material” as used herein refers to all forms of collagen, including those that have been processed or otherwise modified.

Collagen of any type, including, but not limited to, types I, II, III, IV, or any combination thereof, may be used in the compositions of the invention, although type I is generally preferred. Either atelopeptide or telopeptide-containing collagen may be used; however, when collagen from a source, such as bovine collagen, is used, atelopeptide collagen is generally preferred, because of its reduced immunogenicity compared to telopeptide-containing collagen.

Collagen that has not been previously crosslinked by methods such as heat, irradiation, or chemical crosslinking agents is preferred for use in the compositions of the invention, although previously crosslinked collagen may be used. Non-crosslinked atelopeptide fibrillar collagen is commercially available from McGhan Medical Corporation (Santa Barbara, Calif.) at collagen concentrations of 35 mg/ml and 65 mg/ml under the trademarks ZYDERM® I Collagen and ZYDERM® II Collagen, respectively. Glutaraldehyde-crosslinked atelopeptide fibrillar collagen is commercially available from McGhan Medical Corporation at a collagen concentration of 35 mg/ml under the trademark ZYPLAST®.

Collagens for use in the present invention are generally, although not necessarily, in aqueous suspension at a concentration between about 20 mg/ml to about 120 mg/ml, preferably between about 30 mg/ml to about 90 mg/ml.

Although intact collagen is preferred, denatured collagen, commonly known as gelatin, can also be used in the compositions of the invention. Gelatin may have the added benefit of being degradable faster than collagen.

Because of its greater surface area and greater concentration of reactive groups, nonfibrillar collagen is generally preferred. The term “nonfibrillar collagen” refers to any modified or unmodified collagen material that is in substantially nonfibrillar form at pH 7, as indicated by optical clarity of an aqueous suspension of the collagen.

Collagen that is already in nonfibrillar form may be used in the compositions of the invention. As used herein, the term “nonfibrillar collagen” is intended to encompass collagen types that are nonfibrillar in native form, as well as collagens that have been chemically modified such that they are in nonfibrillar form at or around neutral pH. Collagen types that are nonfibrillar (or microfibrillar) in native form include types IV, VI, and VII.

Chemically modified collagens that are in nonfibrillar form at neutral pH include succinylated collagen, propylated collagen, ethylated collagen, methylated collagen, and the like, both of which can be prepared according to the methods described in U.S. Pat. No. 4,164,559, to Miyata et al., which is hereby incorporated by reference in its entirety. Due to its inherent tackiness, methylated collagen is particularly preferred, as disclosed in U.S. Pat. No. 5,614,587 to Rhee et al.

Collagens for use in the crosslinkable compositions of the present invention may start out in fibrillar form, then be rendered nonfibrillar by the addition of one or more fiber disassembly agents. The fiber disassembly agent must be present in an amount sufficient to render the collagen substantially nonfibrillar at pH 7, as described above. Fiber disassembly agents for use in the present invention include, without limitation, various biocompatible alcohols, amino acids, inorganic salts, and carbohydrates, with biocompatible alcohols being particularly preferred. Preferred biocompatible alcohols include glycerol and propylene glycol. Non-biocompatible alcohols, such as ethanol, methanol, and isopropanol, are not preferred for use in the present invention, due to their potentially deleterious effects on the body of the patient receiving them. Preferred amino acids include arginine. Preferred inorganic salts include sodium chloride and potassium chloride. Although carbohydrates, such as various sugars including sucrose, may be used in the practice of the present invention, they are not as preferred as other types of fiber disassembly agents because they can have cytotoxic effects in vivo.

As fibrillar collagen has less surface area and a lower concentration of reactive groups than nonfibrillar, fibrillar collagen is less preferred. However, as disclosed in U.S. Pat. No. 5,614,587, fibrillar collagen, or mixtures of nonfibrillar and fibrillar collagen, may be preferred for use in compositions intended for long-term persistence in vivo, if optical clarity is not a requirement.

Synthetic hydrophilic polymers may also be used in the present invention. Useful synthetic hydrophilic polymers include, but are not limited to: polyalkylene oxides, particularly polyethylene glycol and poly(ethylene oxide)-poly(propylene oxide) copolymers, including block and random copolymers; polyols such as glycerol, polyglycerol (particularly highly branched polyglycerol), propylene glycol and trimethylene glycol substituted with one or more polyalkylene oxides, e.g., mono-, di- and tri-polyoxyethylated glycerol, mono- and di-polyoxyethylated propylene glycol, and mono- and di-polyoxyethylated trimethylene glycol; polyoxyethylated sorbitol, polyoxyethylated glucose; acrylic acid polymers and analogs and copolymers thereof, such as polyacrylic acid per se, polymethacrylic acid, poly(hydroxyethyl-methacrylate), poly(hydroxyethylacrylate), poly(methylalkylsulfoxide methacrylate), poly(methylalkylsulfoxide acrylate) and copolymers of any of the foregoing, and/or with additional acrylate species such as aminoethyl acrylate and mono-2-(acryloxy)-ethyl succinate; polymaleic acid; poly(acrylamides) such as polyacrylamide per se, poly(methacrylamide), poly(dimethylacrylamide), and poly(N-isopropyl-acrylamide); poly(olefinic alcohol)s such as poly(vinyl alcohol); poly(N-vinyl lactams) such as poly(vinyl pyrrolidone), poly(N-vinyl caprolactam), and copolymers thereof; polyoxazolines, including poly(methyloxazoline) and poly(ethyloxazoline); and polyvinylamines. It must be emphasized that the aforementioned list of polymers is not exhaustive, and a variety of other synthetic hydrophilic polymers may be used, as will be appreciated by those skilled in the art.

The Crosslinkable Components:

The compositions of the invention also comprise a plurality of crosslinkable components. Each of the crosslinkable components participates in a reaction that results in a crosslinked matrix. Prior to completion of the crosslinking reaction, the crosslinkable components provide the necessary adhesive qualities that enable the methods of the invention.

The crosslinkable components are selected so that crosslinking gives rise to a biocompatible, nonimmunogenic matrix useful in a variety of contexts including adhesion prevention, biologically active agent delivery, tissue augmentation, and other applications. The crosslinkable components of the invention comprise: a component A, which has m nucleophilic groups, wherein m≧2 and a component B, which has n electrophilic groups capable of reaction with the m nucleophilic groups, wherein n≧2 and m+n≧4. An optional third component, optional component C, which has at least one functional group that is either electrophilic and capable of reaction with the nucleophilic groups of component A, or nucleophilic and capable of reaction with the electrophilic groups of component B may also be present. Thus, the total number of functional groups present on components A, B and C, when present, in combination is ≧5; that is, the total functional groups given by m+n+p must be ≧5, where p is the number of functional groups on component C and, as indicated, is ≧1. Each of the components is biocompatible and nonimmunogenic, and at least one component is comprised of a hydrophilic polymer. Also, as will be appreciated, the composition may contain additional crosslinkable components D, E, F, etc., having one or more reactive nucleophilic or electrophilic groups and thereby participate in formation of the crosslinked biomaterial via covalent bonding to other components.

The m nucleophilic groups on component A may all be the same, or, alternatively, A may contain two or more different nucleophilic groups. Similarly, the n electrophilic groups on component B may all be the same, or two or more different electrophilic groups may be present. The functional group(s) on optional component C, if nucleophilic, may or may not be the same as the nucleophilic groups on component A, and, conversely, if electrophilic, the functional group(s) on optional component C may or may not be the same as the electrophilic groups on component B.

Accordingly, the components may be represented by the structural formulae
R¹(-[Q¹]_{q}-X)ₘ (component A), (I)
R²(-[Q²]ᵣ-Y)ₙ (component B), and (II)
R³(-[Q³]ₛ-Fn)ₚ (optional component C), (III)
wherein:
R¹, R² and R³ are independently selected from the group consisting of C₂ to C₁₄ hydrocarbyl, heteroatom-containing C₂ to C₁₄ hydrocarbyl, hydrophilic polymers, and hydrophobic polymers, providing that at least one of R¹, R² and R³ is a hydrophilic polymer, preferably a synthetic hydrophilic polymer;
X represents one of the m nucleophilic groups of component A, and the various X moieties on A may be the same or different;
Y represents one of the n electrophilic groups of component B, and the various Y moieties on A may be the same or different;
Fn represents a functional group on optional component C;
Q¹, Q² and Q³ are linking groups;
m≧2, n≧2, m+n is ≧4, q, and r are independently zero or 1, and when optional component C is present, p≧1, and s is independently zero or 1.

Reactive Groups:

X may be virtually any nucleophilic group, so long as reaction can occur with the electrophilic group Y. Analogously, Y may be virtually any electrophilic group, so long as reaction can take place with X. The only limitation is a practical one, in that reaction between X and Y should be fairly rapid and take place automatically upon admixture with an aqueous medium, without need for heat or potentially toxic or non-biodegradable reaction catalysts or other chemical reagents. It is also preferred although not essential that reaction occur without need for ultraviolet or other radiation. Ideally, the reactions between X and Y should be complete in under 60 minutes, preferably under 30 minutes. Most preferably, the reaction occurs in about 5 to 15 minutes or less.

Examples of nucleophilic groups suitable as X include, but are not limited to, —NH₂, —NHR⁴, —N(R⁴)₂, —SH, —OH, —COOH, —C₆H₄—OH, —PH₂, —PHR⁵, —P(R⁵)₂, —NH—NH₂, —CO—NH—NH₂, —C₅H₄N, etc. wherein R⁴ and R⁵ are hydrocarbyl, typically alkyl or monocyclic aryl, preferably alkyl, and most preferably lower alkyl. Organometallic moieties are also useful nucleophilic groups for the purposes of the invention, particularly those that act as carbanion donors. Organometallic nucleophiles are not, however, preferred. Examples of organometallic moieties include: Grignard functionalities —R⁶MgHal wherein R⁶ is a carbon atom (substituted or unsubstituted), and Hal is halo, typically bromo, iodo or chloro, preferably bromo; and lithium-containing functionalities, typically alkyllithium groups; sodium-containing functionalities.

It will be appreciated by those of ordinary skill in the art that certain nucleophilic groups must be activated with a base so as to be capable of reaction with an electrophile. For example, when there are nucleophilic sulfhydryl and hydroxyl groups in the crosslinkable composition, the composition must be admixed with an aqueous base in order to remove a proton and provide an —S⁻ or —O⁻ species to enable reaction with an electrophile. Unless it is desirable for the base to participate in the crosslinking reaction, a nonnucleophilic base is preferred. In some embodiments, the base may be present as a component of a buffer solution. Suitable bases and corresponding crosslinking reactions are described infra in Section E.

The selection of electrophilic groups provided within the crosslinkable composition, i.e., on component B, must be made so that reaction is possible with the specific nucleophilic groups. Thus, when the X moieties are amino groups, the Y groups are selected so as to react with amino groups. Analogously, when the X moieties are sulfhydryl moieties, the corresponding electrophilic groups are sulfhydryl-reactive groups, and the like.

By way of example, when X is amino (generally although not necessarily primary amino), the electrophilic groups present on Y are amino reactive groups such as, but not limited to: (1) carboxylic acid esters, including cyclic esters and “activated” esters; (2) acid chloride groups (—CO—Cl); (3) anhydrides (—(CO)—O—(CO)—R); (4) ketones and aldehydes, including α,β-unsaturated aldehydes and ketones such as —CH═CH—CH═O and —CH═CH—C(CH₃)═O; (5) halides; (6) isocyanate (—N═C═O); (7) isothiocyanate (—N═C═S); (8) epoxides; (9) activated hydroxyl groups (e.g., activated with conventional activating agents such as carbonyidiimidazole or sulfonyl chloride); and (10) olefins, including conjugated olefins, such as ethenesulfonyl (—SO₂CH═CH₂) and analogous functional groups, including acrylate (—CO₂—C═CH₂), methacrylate (—CO₂—C(CH₃)═CH₂)), ethyl acrylate (—CO₂—C(CH₂CH₃)═CH₂), and ethyleneimino (—CH═CH—C═NH). Since a carboxylic acid group per se is not susceptible to reaction with a nucleophilic amine, components containing carboxylic acid groups must be activated so as to be amine-reactive. Activation may be accomplished in a variety of ways, but often involves reaction with a suitable hydroxyl-containing compound in the presence of a dehydrating agent such as dicyclohexylcarbodiimide (DCC) or dicyclohexylurea (DHU). For example, a carboxylic acid can be reacted with an alkoxy-substituted N-hydroxy-succinimide or N-hydroxysulfosuccinimide in the presence of DCC to form reactive electrophilic groups, the N-hydroxysuccinimide ester and the N-hydroxysulfosuccinimide ester, respectively. Carboxylic acids may also be activated by reaction with an acyl halide such as an acyl chloride (e.g., acetyl chloride), to provide a reactive anhydride group. In a further example, a carboxylic acid may be converted to an acid chloride group using, e.g., thionyl chloride or an acyl chloride capable of an exchange reaction. Specific reagents and procedures used to carry out such activation reactions will be known to those of ordinary skill in the art and are described in the pertinent texts and literature.

Analogously, when X is sulfhydryl, the electrophilic groups present on Y are groups that react with a sulfhydryl moiety. Such reactive groups include those that form thioester linkages upon reaction with a sulfhydryl group, such as those described in PCT Publication No. WO 00/62827 to Wallace et al. As explained in detail therein, such “sulfhydryl reactive” groups include, but are not limited to: mixed anhydrides; ester derivatives of phosphorus; ester derivatives of p-nitrophenol, p-nitrothiophenol and pentafluorophenol; esters of substituted hydroxyla mines, including N-hydroxyphthalimide esters, N-hydroxysuccinimide esters, N-hydroxysulfosuccinimide esters, and N-hydroxyglutarimide esters; esters of 1-hydroxybenzotriazole; 3-hydroxy-3,4-dihydro-benzotriazin-4-one; 3-hydroxy-3,4-dihydro-quinazoline-4-one; carbonylimidazole derivatives; acid chlorides; ketenes; and isocyanates. With these sulfhydryl reactive groups, auxiliary reagents can also be used to facilitate bond formation, e.g., 1-ethyl-3-[3-dimethylaminopropyl]carbodiimide can be used to facilitate coupling of sulfhydryl groups to carboxyl-containing groups.

In addition to the sulfhydryl reactive groups that form thioester linkages, various other sulfhydryl reactive functionalities can be utilized that form other types of linkages. For example, compounds that contain methyl imidate derivatives form imido-thioester linkages with sulfhydryl groups. Alternatively, sulfhydryl reactive groups can be employed that form disulfide bonds with sulfhydryl groups; such groups generally have the structure —S—S—Ar where Ar is a substituted or unsubstituted nitrogen-containing heteroaromatic moiety or a non-heterocyclic aromatic group substituted with an electron-withdrawing moiety, such that Ar may be, for example, 4-pyridinyl, o-nitrophenyl, m-nitrophenyl, p-nitrophenyl, 2,4-dinitrophenyl, 2-nitro-4-benzoic acid, 2-nitro-4-pyridinyl, etc. In such instances, auxiliary reagents, i.e., mild oxidizing agents such as hydrogen peroxide, can be used to facilitate disulfide bond formation.

Yet another class of sulfhydryl reactive groups forms thioether bonds with sulfhydryl groups. Such groups include, inter alia, maleimido, substituted maleimido, haloalkyl, epoxy, imino, and aziridino, as well as olefins (including conjugated olefins) such as ethenesulfonyl, etheneimino, acrylate, methacrylate, and α,β-unsaturated aldehydes and ketones. This class of sulfhydryl reactive groups are particularly preferred as the thioether bonds may provide faster crosslinking and longer in vivo stability.

When X is —OH, the electrophilic functional groups on the remaining component(s) must react with hydroxyl groups. The hydroxyl group may be activated as described above with respect to carboxylic acid groups, or it may react directly in the presence of base with a sufficiently reactive electrophile such as an epoxide group, an aziridine group, an acyl halide, or an anhydride.

When X is an organometallic nucleophile such as a Grignard functionality or an alkyllithium group, suitable electrophilic functional groups for reaction therewith are those containing carbonyl groups, including, by way of example, ketones and aldehydes.

It will also be appreciated that certain functional groups can react as nucleophiles or as electrophiles, depending on the selected reaction partner and/or the reaction conditions. For example, a carboxylic acid group can act as a nucleophile in the presence of a fairly strong base, but generally acts as an electrophile allowing nucleophilic attack at the carbonyl carbon and concomitant replacement of the hydroxyl group with the incoming nucleophile.

The covalent linkages in the crosslinked structure that result upon covalent binding of specific nucleophilic components to specific electrophilic components in the crosslinkable composition include, solely by way of example, the following (the optional linking groups Q¹ and Q² are omitted for clarity):

TABLE

| REPRESENTATIVE | | |
|---|---|---|
| NUCLEOPHILIC | | |
| COMPONENT | REPRESENTATIVE | |
| (A, optional | ELECTROPHILIC | |
| component C | COMPONENT | |
| element FN_{NU}) | (B, FN_{EL}) | RESULTING LINKAGE |
| R¹—NH₂ | R²—O—(CO)—O—N(COCH₂) | R¹—NH—(CO)—O—R² |
| | (succinimidyt carbonate | |
| | terminus) | |
| R¹—SH | R²—O—(CO)—O—N(COCH₂) | R¹—S—(CO)—O—R² |
| R¹—OH | R²—O—(CO)—O—N(COCH₂) | R¹—O—(CO)—R² |
| R¹—NH₂ | R²—O(CO)—CH═CH₂ | R¹—NH—CH₂CH₂—(CO)—O—R² |
| | (acrylate terminus) | |
| R¹—SH | R²—O—(CO)—CH═CH₂ | R¹—S—CH₂CH₂—(CO)—O—R² |
| R¹—OH | R²—O—(CO)—CH═CH₂ | R¹—O—CH₂CH₂—(CO)—O—R² |
| R¹—NH₂ | R²—O(CO)—(CH₂)₃—CO₂— | R¹—NH—(CO)—(CH₂)₃—(CO)— |
| | N(COCH₂) | OR² |
| | (succinimidyl glutarate | |
| | terminus) | |
| R¹—SH | R²—O(CO)—(CH₂)₃—CO₂— | R¹—S—(CO)—(CH₂)₃—(CO)— |
| | N(COCH₂) | OR² |
| R¹—OH | R²—O(CO)—(CH₂)₃—CO₂— | R¹—O—(CO)—(CH₂)₃—(CO)— |
| | N(COCH₂) | OR² |
| R¹—NH₂ | R²—O—CH₂—CO₂—N(COCH₂) | R¹—NH—(CO)—CH₂—OR² |
| | (succinimidyl acetate | |
| | terminus) | |
| R¹—SH | R²—O—CH₂—CO₂—N(COCH₂) | R¹—S—(CO)—CH₂—0R² |
| R¹—OH | R²—O—CH₂—CO₂—N(COCH₂) | R¹—O—(CO)—CH₂—0R² |
| R¹—NH₂ | R²—O—NH(CO)—(CH₂)₂—CO₂— | R¹—NH—(CO)—(CH₂)₂—(CO)— |
| | N(COCH₂) | NH—OR |
| | (succinimidyl succinamide | |
| | terminus) | |
| R¹—SH | R²—O—NH(CO)—(CH₂)₂—CO₂— | R¹—S—(CO)—(CH₂)₂—(CO)— |
| | N(COCH₂) | NH—OR² |
| R¹—OH | R²—O—NH(CO)—(CH₂)₂—CO₂— | R¹—O—(CO)—(CH₂)₂—(CO)— |
| | N(COCH₂) | NH—OR² |
| R¹—NH₂ | R²—O—(CH₂)₂—CHO | R¹—NH—(CO)—(CH₂)₂—OR² |
| | (propionaldehyde terminus) | |
| R¹—NH₂ | 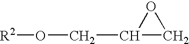 | R¹—NH—CH₂—CH(OH)—CH₂—OR² and R¹—N[CH₂—CH(OH)—CH₂—OR²]₂ |
| | (glycidyl ether terminus) | |
| R¹—NH₂ | R²—O—(CH₂)₂—N═C═O | R¹—NH—(CO)—NH—CH₂—OR² |
| | (isocyanate terminus) | |
| R¹—NH₂ | R²—SO₂—CH═CH₂ | R¹—NH—CH₂CH₂—SO₂—R² |
| | (vinyl sulfone terminus) | |
| R¹—SH | R²—SO₂—CH═CH₂ | R¹—S—CH₂CH₂—SO₂—R² |

Linking Groups:

The functional groups X and Y and FN on optional component C may be directly attached to the compound core (R¹, R² or R³on optional component C, respectively), or they may be indirectly attached through a linking group, with longer linking groups also termed “chain extenders.” In structural formulae (I), (II) and (III), the optional linking groups are represented by Q¹, Q² and Q³, wherein the linking groups are present when q, r and s are equal to 1 (with R, X, Y, Fn, m n and p as defined previously).

Suitable linking groups are well known in the art. See, for example, International Patent Publication No. WO 97/22371. Linking groups are useful to avoid steric hindrance problems that are sometimes associated with the formation of direct linkages between molecules. Linking groups may additionally be used to link several multifunctionally activated compounds together to make larger molecules. In a preferred embodiment, a linking group can be used to alter the degradative properties of the compositions after administration and resultant gel formation. For example, linking groups can be incorporated into components A, B, or optional component C to promote hydrolysis, to discourage hydrolysis, or to provide a site for enzymatic degradation.

Examples of linking groups that provide hydrolyzable sites, include, inter alia: ester linkages; anhydride linkages, such as obtained by incorporation of glutarate and succinate; ortho ester linkages; ortho carbonate linkages such as trimethylene carbonate; amide linkages; phosphoester linkages; α-hydroxy acid linkages, such as may be obtained by incorporation of lactic acid and glycolic acid; lactone-based linkages, such as may be obtained by incorporation of caprolactone, valerolactone, γ-butyrolactone and p-dioxanone; and amide linkages such as in a dimeric, oligomeric, or poly(amino acid) segment. Examples of non-degradable linking groups include succinimide, propionic acid and carboxymethylate linkages. See, for example, PCT WO 99/07417. Examples of enzymatically degradable linkages include Leu-Gly-Pro-Ala, which is degraded by collagenase; and Gly-Pro-Lys, which is degraded by plasmin.

Linking groups can also enhance or suppress the reactivity of the various nucleophilic and electrophilic groups. For example, electron-withdrawing groups within one or two carbons of a sulfhydryl group would be expected to diminish its effectiveness in coupling, due to a lowering of nucleophilicity. Carbon-carbon double bonds and carbonyl groups will also have such an effect. Conversely, electron-withdrawing groups adjacent to a carbonyl group (e.g., the reactive carbonyl of glutaryl-N-hydroxysuccinimidyl) would increase the reactivity of the carbonyl carbon with respect to an incoming nucleophile. By contrast, sterically bulky groups in the vicinity of a functional group can be used to diminish reactivity and thus coupling rate as a result of steric hindrance.

By way of example, particular linking groups and corresponding component structure are indicated in the following Table:

TABLE

| LINKING GROUP | COMPONENT STRUCTURE |
|---|---|
| —O—(CH₂)ₙ— | Component A: R¹—O—(CH₂)ₙ—X |
| | Component B: R²—O—(CH₂)ₙ—Y |
| | Optional Component C: R³—O—(CH₂)ₙ—Z |
| —S—(CH₂)ₙ— | Component A: R¹—S—(CH₂)ₙ—X |
| | Component B: R²—S—(CH₂)ₙ—Y |
| | Optional Component C: R³—S—(CH₂)ₙ—Z |
| —NH—(CH₂)ₙ— | Component A: R¹—NH—(CH₂)ₙ—X |
| | Component B: R²—NH—(CH₂)ₙ—Y |
| | Optional Component C: R³—NH—(CH₂)ₙ—Z |
| —O—(CO)—NH—(CH₂)ₙ— | Component A: R¹—O—(CO)—NH—(CH₂)ₙ—X |
| | Component B: R²—O—(CO)—NH—(CH₂)ₙ—Y |
| | Optional Component C: R³—O—(CO)—NH—(CH₂)ₙ—Z |
| —NH—(CO)—O—(CH₂)ₙ— | Component A: R¹—NH—(CO)—O—(CH₂)ₙ—X |
| | Component B: R²—NH—(CO)—O—(CH₂)ₙ—Y |
| | Optional Component C: R³—NH—(CO)—O—(CH₂)ₙ—Z |
| —O—(CO)—(CH₂)ₙ— | Component A: R¹—O—(CO)—(CH₂)ₙ—X |
| | Component B: R²—O—(CO)—(CH₂)ₙ—Y |
| | Optional Component C: R³—O—(CO)—(CH₂)ₙ—Z |
| —(CO)—O—CH₂)ₙ— | Component A: R¹—(CO)—O—(CH₂)ₙ—X |
| | Component B: R²—(CO)—O—(CH₂)ₙ—Y |
| | Optional Component C: R³—(CO)—O—(CH₂)ₙ—Z |
| —O—(CO)—O—(CH₂)ₙ— | Component A: R¹—O—(CO)—O—(CH₂)ₙ—X |
| | Component B: R²—O—(CO)—O—(CH₂)ₙ—Y |
| | Optional Component C: R³—O—(CO)—O—(CH₂)ₙ—Z |
| —O—(CO)—CHR⁷— | Component A: R¹—O—(CO)—CHR⁷—X |
| | Component B: R²—O—(CO)—CHR⁷—Y |
| | Optional Component C: R³—O—(CO)—CHR⁷—Z |
| —O—R⁸—(CO)—NH— | Component A: R¹—O—R⁸—(CO)—NH—X |
| | Component B: R²—O—R⁸—(CO)—NH—Y |
| | Optional Component C: R³—O—R⁸—(CO)—NH—Z |

In the above Table, n is generally in the range of 1 to about 10, R⁷ is generally hydrocarbyl, typically alkyl or aryl, preferably alkyl, and most preferably lower alkyl, and R⁸ is hydrocarbylene, heteroatom-containing hydrocarbylene, substituted hydrocarbylene, or substituted heteroatom-containing hydrocarbylene) typically alkylene or arylene (again, optionally substituted and/or containing a heteroatom), preferably lower alkylene (e.g., methylene, ethylene, n-propylene, n-butylene, etc.), phenylene, or amidoalkylene (e.g., —(CO)—NH—CH₂).

Other general principles that should be considered with respect to linking groups are as follows: If higher molecular weight components are to be used, they preferably have biodegradable linkages as described above, so that fragments larger than 20,000 mol. wt. are not generated during resorption in the body. In addition, to promote water miscibility and/or solubility, it may be desired to add sufficient electric charge or hydrophilicity. Hydrophilic groups can be easily introduced using known chemical synthesis, so long as they do not give rise to unwanted swelling or an undesirable decrease in compressive strength. In particular, polyalkoxy segments may weaken gel strength.

The Component Core:

The “core” of each crosslinkable component is comprised of the molecular structure to which the nucleophilic or electrophilic groups are bound. Using the formulae (I) R¹-[Q¹]_{q}-X)ₘ, for component A, (II) R²(-[Q²]ᵣ-Y)ₙ for component B, and (III)

R³(-[Q³]-Fn)ₚ for optional component C, the “core” groups are R¹, R² and R³. Each molecular core of the reactive components of the crosslinkable composition is generally selected from synthetic and naturally occurring hydrophilic polymers, hydrophobic polymers, and C₂-C₁₄ hydrocarbyl groups zero to 2 heteroatoms selected from N, O and S, with the proviso that at least one of the crosslinkable components A, B, and optionally C, comprises a molecular core of a synthetic hydrophilic polymer. In a preferred embodiment, at least one of A and B comprises a molecular core of a synthetic hydrophilic polymer.

Hydrophilic Crosslinkable Components

In one aspect, the crosslinkable component(s) is (are) hydrophilic polymers. The term “hydrophilic polymer” as used herein refers to a synthetic polymer having an average molecular weight and composition effective to render the polymer “hydrophilic” as defined above. As discussed above, synthetic crosslinkable hydrophilic polymers useful herein include, but are not limited to: polyalkylene oxides, particularly polyethylene glycol and poly(ethylene oxide)-poly(propylene oxide) copolymers, including block and random copolymers; polyols such as glycerol, polyglycerol (particularly highly branched polyglycerol), propylene glycol and trimethylene glycol substituted with one or more polyalkylene oxides, e.g., mono-, di- and tri-polyoxyethylated glycerol, mono- and di-polyoxyethylated propylene glycol, and mono- and di-polyoxyethylated trimethylene glycol; polyoxyethylated sorbitol, polyoxyethylated glucose; acrylic acid polymers and analogs and copolymers thereof, such as polyacrylic acid per se, polymethacrylic acid, poly(hydroxyethyl-methacrylate), poly(hydroxyethylacrylate), poly(methylalkylsulfoxide methacrylate), poly(methylalkylsulfoxide acrylate) and copolymers of any of the foregoing, and/or with additional acrylate species such as aminoethyl acrylate and mono-2-(acryloxy)-ethyl succinate; polymaleic acid; poly(acrylamides) such as polyacrylamide per se, poly(methacrylamide), poly(dimethylacrylamide), and poly(N-isopropyl-acrylamide); poly(olefinic alcohol)s such as poly(vinyl alcohol); poly(N-vinyl lactams) such as poly(vinyl pyrrolidone), poly(N-vinyl caprolactam), and copolymers thereof; polyoxazolines, including poly(methyloxazoline) and poly(ethyloxazoline); and polyvinylamines. It must be emphasized that the aforementioned list of polymers is not exhaustive, and a variety of other synthetic hydrophilic polymers may be used, as will be appreciated by those skilled in the art.

The synthetic crosslinkable hydrophilic polymer may be a homopolymer, a block copolymer, a random copolymer, or a graft copolymer. In addition, the polymer may be linear or branched, and if branched, may be minimally to highly branched, dendrimeric, hyperbranched, or a star polymer. The polymer may include biodegradable segments and blocks, either distributed throughout the polymer's molecular structure or present as a single block, as in a block copolymer. Biodegradable segments are those that degrade so as to break covalent bonds. Typically, biodegradable segments are segments that are hydrolyzed in the presence of water and/or enzymatically cleaved in situ. Biodegradable segments may be composed of small molecular segments such as ester linkages, anhydride linkages, ortho ester linkages, ortho carbonate linkages, generally be composed of oligomeric or polymeric segments incorporated within the hydrophilic polymer. Illustrative oligomeric and polymeric segments that are biodegradable include, by way of example, poly(amino acid) segments, poly(orthoester) segments, poly(orthocarbonate) segments, and the like.

Other suitable synthetic crosslinkable hydrophilic polymers include chemically synthesized polypeptides, particularly polynucleophilic polypeptides that have been synthesized to incorporate amino acids containing primary amino groups (such as lysine) and/or amino acids containing thiol groups (such as cysteine). Poly(lysine), a synthetically produced polymer of the amino acid lysine (145 MW), is particularly preferred. Poly(lysine)s have been prepared having anywhere from 6 to about 4,000 primary amino groups, corresponding to molecular weights of about 870 to about 580,000. Poly(lysine)s for use in the present invention preferably have a molecular weight within the range of about 1,000 to about 300,000, more preferably within the range of about 5,000 to about 100,000, and most preferably, within the range of about 8,000 to about 15,000. Poly(lysine)s of varying molecular weights are commercially available from Peninsula Laboratories, Inc. (Belmont, Calif.).

The synthetic crosslinkable hydrophilic polymer may be a homopolymer, a block copolymer, a random copolymer, or a graft copolymer. In addition, the polymer may be linear or branched, and if branched, may be minimally to highly branched, dendrimeric, hyperbranched, or a star polymer. The polymer may include biodegradable segments and blocks, either distributed throughout the polymer's molecular structure or present as a single block, as in a block copolymer. Biodegradable segments are those that degrade so as to break covalent bonds. Typically, biodegradable segments are segments that are hydrolyzed in the presence of water and/or enzymatically cleaved in situ. Biodegradable segments may be composed of small molecular segments such as ester linkages, anhydride linkages, ortho ester linkages, ortho carbonate linkages, amide linkages, phosphonate linkages, etc. Larger biodegradable “blocks” will generally be composed of oligomeric or polymeric segments incorporated within the hydrophilic polymer. Illustrative oligomeric and polymeric segments that are biodegradable include, by way of example, poly(amino acid) segments, poly(orthoester) segments, poly(orthocarbonate) segments, and the like.

Although a variety of different synthetic crosslinkable hydrophilic polymers can be used in the present compositions, as indicated above, preferred synthetic crosslinkable hydrophilic polymers are polyethylene glycol (PEG) and polyglycerol (PG), particularly highly branched polyglycerol. Various forms of PEG are extensively used in the modification of biologically active molecules because PEG lacks toxicity, antigenicity, and immunogenicity (i.e., is biocompatible), can be formulated so as to have a wide range of solubilities, and do not typically interfere with the enzymatic activities and/or conformations of peptides. A particularly preferred synthetic crosslinkable hydrophilic polymer for certain applications is a polyethylene glycol (PEG) having a molecular weight within the range of about 100 to about 100,000 mol. wt., although for highly branched PEG, far higher molecular weight polymers can be employed—up to 1,000,000 or more—providing that biodegradable sites are incorporated ensuring that all degradation products will have a molecular weight of less than about 30,000. For most PEGs, however, the preferred molecular weight is about 1,000 to about 20,000 mol. wt., more preferably within the range of about 7,500 to about 20,000 mol. wt. Most preferably, the polyethylene glycol has a molecular weight of approximately 10,000 mol. wt.

Naturally occurring crosslinkable hydrophilic polymers include, but are not limited to: proteins such as collagen, fibronectin, albumins, globulins, fibrinogen, and fibrin, with collagen particularly preferred; carboxylated polysaccharides such as polymannuronic acid and polygalacturonic acid; aminated polysaccharides, particularly the glycosaminoglycans, e.g., hyaluronic acid, chitin, chondroitin sulfate A, B, or C, keratin sulfate, keratosulfate and heparin; and activated polysaccharides such as dextran and starch derivatives. Collagen and glycosaminoglycans are examples of naturally occurring hydrophilic polymers for use herein, with methylated collagen being a preferred hydrophilic polymer.

Any of the hydrophilic polymers herein must contain, or be activated to contain, functional groups, i.e., nucleophilic or electrophilic groups, which enable crosslinking. Activation of PEG is discussed below; it is to be understood, however, that the following discussion is for purposes of illustration and analogous techniques may be employed with other polymers.

With respect to PEG, first of all, various functionalized polyethylene glycols have been used effectively in fields such as protein modification (see Abuchowski et al., Enzymes as Drugs, John Wiley & Sons: New York, N.Y. (1981) pp. 367-383; and Dreborg et al., Crit. Rev. Therap. Drug Carrier Syst. (1990) 6:315), peptide chemistry (see Mutter et al., The Peptides, Academic: New York, N.Y. 2:285-332; and Zalipsky et al., Int. J. Peptide Protein Res. (1987) 30:740), and the synthesis of polymeric drugs (see Zalipsky et al., Eur. Polym. J. (1983) 19:1177; and Ouchi et al., J. Macromol. Sci. Chem. (1987) A24:1011).

Activated forms of PEG, including multifunctionally activated PEG, are commercially available, and are also easily prepared using known methods. For example, see Chapter 22 of Poly(ethylene Glycol) Chemistry: Biotechnical and Biomedical Applications, J. Milton Harris, ed., Plenum Press, NY (1992); and Shearwater Polymers, Inc. Catalog, Polyethylene Glycol Derivatives, Huntsville, Ala. (1997-1998).

Structures for some specific, tetrafunctionally activated forms of PEG are shown in FIGS. 1 to 10 of U.S. Pat. No. 5,874,500, as are generalized reaction products obtained by reacting the activated PEGs with multi-amino PEGs, i.e., a PEG with two or more primary amino groups. The activated PEGs illustrated have a pentaerythritol (2,2-bis(hydroxymethyl)-1,3-propanediol) core. Such activated PEGs, as will be appreciated by those in the art, are readily prepared by conversion of the exposed hydroxyl groups in the PEGylated polyol (i.e., the terminal hydroxyl groups on the PEG chains) to carboxylic acid groups (typically by reaction with an anhydride in the presence of a nitrogenous base), followed by esterification with N-hydroxysuccinimide, N-hydroxysulfosuccinimide, or the like, to give the polyfunctionally activated PEG.

Hydrophobic Polymers:

The crosslinkable compositions of the invention can also include hydrophobic polymers, although for most uses hydrophilic polymers are preferred. Polylactic acid and polyglycolic acid are examples of two hydrophobic polymers that can be used. With other hydrophobic polymers, only short-chain oligomers should be used, containing at most about 14 carbon atoms, to avoid solubility-related problems during reaction.

Low Molecular Weight Components:

As indicated above, the molecular core of one or more of the crosslinkable components can also be a low molecular weight compound, i.e., a C₂-C₁₄ hydrocarbyl group containing zero to 2 heteroatoms selected from N, O, S and combinations thereof. Such a molecular core can be substituted with nucleophilic groups or with electrophilic groups.

When the low molecular weight molecular core is substituted with primary amino groups, the component may be, for example, ethylenediamine (H₂N—CH₂CH₂—NH₂), tetramethylenediamine (H₂N—(CH₄)—NH₂), pentamethylenediamine (cadaverine) (H₂N—(CH₅)—NH₂), hexamethylenediamine (H₂N—(CH₆)—NH₂), bis(2-aminoethyl)amine (HN—[CH₂CH₂—NH₂]₂), or tris(2-aminoethyl)amine (N—[CH₂CH₂—NH₂]₃).

Low molecular weight diols and polyols include trimethylolpropane, di(trimethylol propane), pentaerythritol, and diglycerol, all of which require activation with a base in order to facilitate their reaction as nucleophiles. Such diols and polyols may also be functionalized to provide di- and poly-carboxylic acids, functional groups that are, as noted earlier herein, also useful as nucleophiles under certain conditions. Polyacids for use in the present compositions include, without limitation, trimethylolpropane-based tricarboxylic acid, di(trimethylol propane)-based tetracarboxylic acid, heptanedioic acid, octanedioic acid (suberic acid), and hexadecanedioic acid (thapsic acid), all of which are commercially available and/or readily synthesized using known techniques.

Low molecular weight di- and poly-electrophiles include, for example, disuccinimidyl suberate (DSS), bis(sulfosuccinimidyl) suberate (BS₃), dithiobis(succinimidylpropionate) (DSP), bis(2-succinimidooxycarbonyloxy) ethyl sulfone (BSOCOES), and 3,3′-dithiobis(sulfosuccinimidylpropionate (DTSPP), and their analogs and derivatives. The aforementioned compounds are commercially available from Pierce (Rockford, III.). Such di- and poly-electrophiles can also be synthesized from di- and polyacids, for example by reaction with an appropriate molar amount of N-hydroxysuccinimide in the presence of DCC. Polyols such as trimethylolpropane and di(trimethylol propane) can be converted to carboxylic acid form using various known techniques, then further derivatized by reaction with NHS in the presence of DCC to produce trifunctionally and tetrafunctionally activated polymers.

Delivery Systems:

Suitable delivery systems for the homogeneous dry powder composition (containing at least two crosslinkable polymers) and the two buffer solutions may involve a multi-compartment spray device, where one or more compartments contains the powder and one or more compartments contain the buffer solutions needed to provide for the aqueous environment, so that the composition is exposed to the aqueous environment as it leaves the compartment. Many devices that are adapted for delivery of multi-component tissue sealants/hemostatic agents are well known in the art and can also be used in the practice of the present invention. Alternatively, the composition can be delivered using any type of controllable extrusion system, or it can be delivered manually in the form of a dry powder, and exposed to the aqueous environment at the site of administration.

The homogeneous dry powder composition and the two buffer solutions may be conveniently formed under aseptic conditions by placing each of the three ingredients (dry powder, acidic buffer solution and basic buffer solution) into separate syringe barrels. For example, the composition, first buffer solution and second buffer solution can be housed separately in a multiple-compartment syringe system having a multiple barrels, a mixing head, and an exit orifice. The first buffer solution can be added to the barrel housing the composition to dissolve the composition and form a homogeneous solution, which is then extruded into the mixing head. The second buffer solution can be simultaneously extruded into the mixing head. Finally, the resulting composition can then be extruded through the orifice onto a surface.

For example, the syringe barrels holding the dry powder and the basic buffer may be part of a dual-syringe system, e.g., a double barrel syringe as described in U.S. Pat. No. 4,359,049 to Redl et al. In this embodiment, the acid buffer can be added to the syringe barrel that also holds the dry powder, so as to produce the homogeneous solution. In other words, the acid buffer may be added (e.g., injected) into the syringe barrel holding the dry powder to thereby produce a homogeneous solution of the first and second components. This homogeneous solution can then be extruded into a mixing head, while the basic buffer is simultaneously extruded into the mixing head. Within the mixing head, the homogeneous solution and the basic buffer are mixed together to thereby form a reactive mixture. Thereafter, the reactive mixture is extruded through an orifice and onto a surface (e.g., tissue), where a film is formed, which can function as a sealant or a barrier, or the like. The reactive mixture begins forming a three-dimensional matrix immediately upon being formed by the mixing of the homogeneous solution and the basic buffer in the mixing head. Accordingly, the reactive mixture is preferably extruded from the mixing head onto the tissue very quickly after it is formed so that the three-dimensional matrix forms on, and is able to adhere to, the tissue.

Other systems for combining two reactive liquids are well known in the art, and include the systems described in U.S. Pat. No. 6,454,786 to Holm et al.; U.S. Pat. No. 6,461,325 to Delmotte et al.; U.S. Pat. No. 5,585,007 to Antanavich et al.; U.S. Pat. No. 5,116,315 to Capozzi et al.; and U.S. Pat. No. 4,631,055 to Redl et al.

Storage and Handling:

Because crosslinkable components containing electrophilic groups react with water, the electrophilic component or components are generally stored and used in sterile, dry form to prevent hydrolysis. Processes for preparing synthetic hydrophilic polymers containing multiple electrophilic groups in sterile, dry form are set forth in commonly assigned U.S. Pat. No. 5,643,464 to Rhee et al. For example, the dry synthetic polymer may be compression molded into a thin sheet or membrane, which can then be sterilized using gamma or, preferably, e-beam irradiation. The resulting dry membrane or sheet can be cut to the desired size or chopped into smaller size particulates.

Components containing multiple nucleophilic groups are generally not water-reactive and can therefore be stored either dry or in aqueous solution. If stored as a dry, particulate, solid, the various components of the crosslinkable composition may be blended and stored in a single container. Admixture of all components with water, saline, or other aqueous media should not occur until immediately prior to use.

In an alternative embodiment, the crosslinking components can be mixed together in a single aqueous medium in which they are both unreactive, i.e., such as in a low pH buffer. Thereafter, they can be sprayed onto the targeted tissue site along with a high pH buffer, after which they will rapidly react and form a gel.

Suitable liquid media for storage of crosslinkable compositions include aqueous buffer solutions such as monobasic sodium phosphate/dibasic sodium phosphate, sodium carbonate/sodium bicarbonate, glutamate or acetate, at a concentration of 0.5 to 300 mM. In general, a sulfhydryl-reactive component such as PEG substituted with maleimido groups or succinimidyl esters is prepared in water or a dilute buffer, with a pH of between around 5 to 6. Buffers with pKs between about 8 and 10.5 for preparing a polysulfhydryl component such as sulfhydryl-PEG are useful to achieve fast gelation time of compositions containing mixtures of sulfhydryl-PEG and SG-PEG. These include carbonate, borate and AMPSO (3-[(1,1 -dimethyl-2-hydroxyethyl)amino]2-hydroxy-propane-sulfonic acid). In contrast, using a combination of maleimidyl PEG and sulfhydryl-PEG, a pH of around 5 to 9 is preferred for the liquid medium used to prepare the sulfhydryl PEG.

Collagen+Fibrinogen and/or Thrombin (e.g., Costasis)

In yet another aspect, the polymer composition may include collagen in combination with fibrinogen and/or thrombin. (See, e.g., U.S. Pat. Nos. 5,290,552; 6,096,309; and 5,997,811). For example, an aqueous composition may include a fibrinogen and FXIII, particularly plasma, collagen in an amount sufficient to thicken the composition, thrombin in an amount sufficient to catalyze polymerization of fibrinogen present in the composition, and Ca²⁺ and, optionally, an antifibrinolytic agent in amount sufficient to retard degradation of the resulting adhesive clot. The composition may be formulated as a two-part composition that may be mixed together just prior to use, in which fibrinogen/FXIII and collagen constitute the first component, and thrombin together with an antifibrinolytic agent, and Ca²⁺ constitute the second component.

Plasma, which provides a source of fibrinogen, may be obtained from the patient for which the composition is to be delivered. The plasma can be used “as is” after standard preparation which includes centrifuging out cellular components of blood. Alternatively, the plasma can be further processed to concentrate the fibrinogen to prepare a plasma cryoprecipitate. The plasma cryoprecipitate can be prepared by freezing the plasma for at least about an hour at about −20° C., and then storing the frozen plasma overnight at about 4° C. to slowly thaw. The thawed plasma is centrifuged and the plasma cryoprecipitate is harvested by removing approximately four-fifths of the plasma to provide a cryoprecipitate comprising the remaining one-fifth of the plasma. Other fibrinogen/FXIII preparations may be used, such as cryoprecipitate, patient autologous fibrin sealant, fibrinogen analogs or other single donor or commercial fibrin sealant materials. Approximately 0.5 ml to about 1.0 ml of either the plasma or the plasma-cryoprecipitate provides about 1 to 2 ml of adhesive composition which is sufficient for use in middle ear surgery. Other plasma proteins (e.g., albumin, plasminogen, von Willebrands factor, Factor VIII, etc.) may or may not be present in the fibrinogen/FXII separation due to wide variations in the formulations and methods to derive them.

Collagen, preferably hypoallergenic collagen, is present in the composition in an amount sufficient to thicken the composition and augment the cohesive properties of the preparation. The collagen may be atelopeptide collagen or telopeptide collagen, e.g., native collagen. In addition to thickening the composition, the collagen augments the fibrin by acting as a macromolecular lattice work or scaffold to which the fibrin network adsorbs. This gives more strength and durability to the resulting glue clot with a relatively low concentration of fibrinogen in comparison to the various concentrated autogenous fibrinogen glue formulations (i.e., AFGs).

The form of collagen which is employed may be described as at least “near native” in its structural characteristics. It may be further characterized as resulting in insoluble fibers at a pH above 5; unless crosslinked or as part of a complex composition, e.g., bone, it will generally consist of a minor amount by weight of fibers with diameters greater than 50 nm, usually from about 1 to 25 volume % and there will be substantially little, if any, change in the helical structure of the fibrils. In addition, the collagen composition must be able to enhance gelation in the surgical adhesion composition.

A number of commercially available collagen preparations may be used. ZYDERM Collagen Implant (ZCI) has a fibrillar diameter distribution consisting of 5 to 10 nm diameter fibers at 90% volume content and the remaining 10% with greater than about 50 nm diameter fibers. ZCI is available as a fibrillar slurry and solution in phosphate buffered isotonic saline, pH 7.2, and is injectable with fine gauge needles. As distinct from ZCI, cross-linked collagen available as ZYPLAST may be employed. ZYPLAST is essentially an exogenously crosslinked (glutaraldehyde) version of ZCI. The material has a somewhat higher content of greater than about 50 nm diameter fibrils and remains insoluble over a wide pH range. Crosslinking has the effect of mimicking in vivo endogenous crosslinking found in many tissues.

Thrombin acts as a catalyst for fibrinogen to provide fibrin, an insoluble polymer and is present in the composition in an amount sufficient to catalyze polymerization of fibrinogen present in the patient plasma. Thrombin also activates FXIII, a plasma protein that catalyzes covalent crosslinks in fibrin, rendering the resultant clot insoluble. Usually the thrombin is present in the adhesive composition in concentration of from about 0.01 to about 1000 or greater NIH units (NIHu) of activity, usually about i to about 500 NIHu, most usually about 200 to about 500 NIHu. The thrombin can be from a variety of host animal sources, conveniently bovine. Thrombin is commercially available from a variety of sources including Parke-Davis, usually lyophilized with buffer salts and stabilizers in vials which provide thrombin activity ranging from about 1000 NlHu to 10,000 NlHu. The thrombin is usually prepared by reconstituting the powder by the addition of either sterile distilled water or isotonic saline. Alternately, thrombin analogs or reptile-sourced coagulants may be used.

The composition may additionally comprise an effective amount of an antifibrinolytic agent to enhance the integrity of the glue clot as the healing processes occur. A number of antifibrinolytic agents are well known and include aprotinin, C1-esterase inhibitor and ε-amino-n-caproic acid (EACA). ε-amino-n-caproic acid, the only antifibrinolytic agent approved by the FDA, is effective at a concentration of from about 5 mg/ml to about 40 mg/ml of the final adhesive composition, more usually from about 20 to about 30 mg/ml. EACA is commercially available as a solution having a concentration of about 250 mg/ml. Conveniently, the commercial solution is diluted with distilled water to provide a solution of the desired concentration. That solution is desirably used to reconstitute lyophilized thrombin to the desired thrombin concentration.

Other examples of in situ forming materials based on the crosslinking of proteins are described, e.g., in U.S. Pat. Nos. RE38158; 4,839,345; 5,514,379, 5,583,114; 6,458,147; 6,371,975; 5,290,552; 6,096,309; U.S. patent application Publication Nos. 2002/0161399; 2001/0018598 and PCT Publication Nos. WO 03/090683; WO 01/45761; WO 99/66964 and WO 96/03159).

Self-Reactive Compounds

In one aspect, the therapeutic agent is released from a crosslinked matrix formed, at least in part, from a self-reactive compound. As used herein, a self-reactive compound comprises a core substituted with a minimum of three reactive groups. The reactive groups may be directed attached to the core of the compound, or the reactive groups may be indirectly attached to the compound's core, e.g., the reactive groups are joined to the core through one or more linking groups.

Each of the three reactive groups that are necessarily present in a self-reactive compound can undergo a bond-forming reaction with at least one of the remaining two reactive groups. For clarity it is mentioned that when these compounds react to form a crosslinked matrix, it will most often happen that reactive groups on one compound will reactive with reactive groups on another compound. That is, the term “self-reactive” is not intended to mean that each self-reactive compound necessarily reacts with itself, but rather that when a plurality of identical self-reactive compounds are in combination and undergo a crosslinking reaction, then these compounds will react with one another to form the matrix. The compounds are “self-reactive” in the sense that they can react with other compounds having the identical chemical structure as themselves.

The self-reactive compound comprises at least four components: a core and three reactive groups. In one embodiment, the self-reactive compound can be characterized by the formula (I), where R is the core, the reactive groups are represented by X¹, X² and X³, and a linker (L) is optionally present between the core and a functional group.

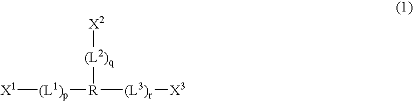

The core R is a polyvalent moiety having attachment to at least three groups (i.e., it is at least trivalent) and may be, or may contain, for example, a hydrophilic polymer, a hydrophobic polymer, an amphiphilic polymer, a C₂₋₁₄ hydrocarbyl, or a C₂₋₁₄ hydrocarbyl which is heteroatom-containing. The linking groups L¹, L², and L³ may be the same or different. The designators p, q and r are either 0 (when no linker is present) or 1 (when a linker is present). The reactive groups X¹, X² and X³ may be the same or different. Each of these reactive groups reacts with at least one other reactive group to form a three-dimensional matrix. Therefore X¹ can react with X² and/or X³, X² can react with X¹ and/or X³, X³ can react with X¹ and/or X² and so forth. A trivalent core will be directly or indirectly bonded to three functional groups, a tetravalent core will be directly or indirectly bonded to four functional groups, etc.

Each side chain typically has one reactive group. However, the invention also encompasses self-reactive compounds where the side chains contain more than one reactive group. Thus, in another embodiment of the invention, the self-reactive compound has the formula (II):
[X′-(L⁴)ₐ-Y′-(L⁵)_{b}]_{c}-R′
where: a and b are integers from 0-1; c is an integer from 3-12; R′ is selected from hydrophilic polymers, hydrophobic polymers, amphiphilic polymers, C₂₋₁₄ hydrocarbyls, and heteroatom-containing C₂₋₁₄ hydrocarbyls; X′ and Y′ are reactive groups and can be the same or different; and L⁴ and L⁵ are linking groups. Each reactive group inter-reacts with the other reactive group to form a three-dimensional matrix. The compound is essentially non-reactive in an initial environment but is rendered reactive upon exposure to a modification in the initial environment that provides a modified environment such that a plurality of the self-reactive compounds inter-react in the modified environment to form a three-dimensional matrix. In one preferred embodiment, R is a hydrophilic polymer. In another preferred embodiment, X′ is a nucleophilic group and Y′ is an electrophilic group.

The following self-reactive compound is one example of a compound of formula (II): 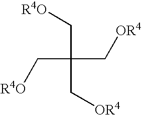
where R⁴ has the formula:

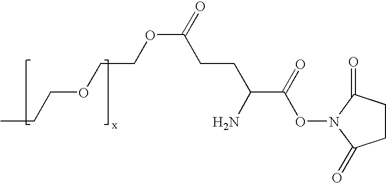

Thus, in formula (II), a and b are 1; c is 4; the core R′ is the hydrophilic polymer, tetrafunctionally activated polyethylene glycol, (C(CH₂—O—)₄; X′ is the electrophilic reactive group, succinimidyl; Y′ is the nucleophilic reactive group —CH—NH₂; L⁴ is —C(O)—O—; and L⁵ is —(CH₂—CH₂—O—CH₂)ₓ—CH₂—O—C(O)—(CH₂)₂—.

The self-reactive compounds of the invention are readily synthesized by techniques that are well known in the art. An exemplary synthesis is set forth below:

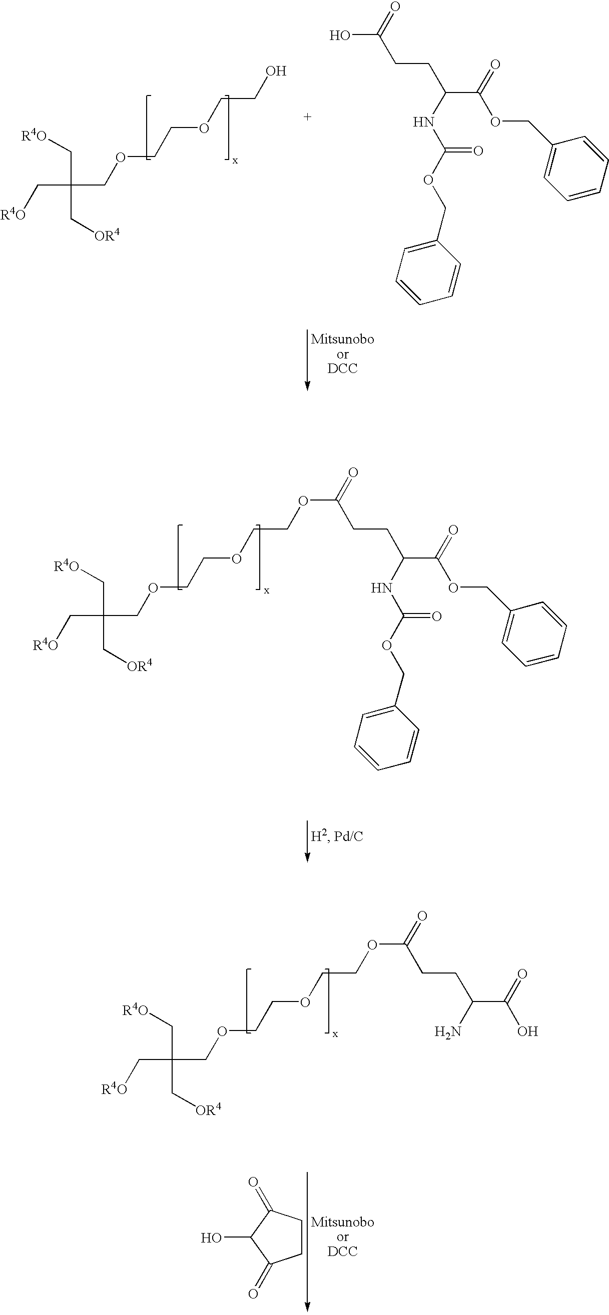

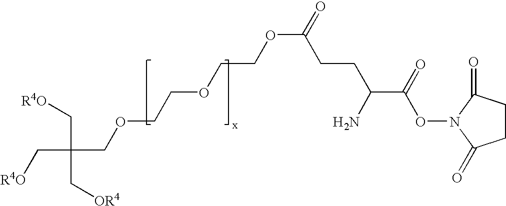

The reactive groups are selected so that the compound is essentially non-reactive in an initial environment. Upon exposure to a specific modification in the initial environment, providing a modified environment, the compound is rendered reactive and a plurality of self-reactive compounds are then able to inter-react in the modified environment to form a three-dimensional matrix. Examples of modification in the initial environment are detailed below, but include the addition of an aqueous medium, a change in pH, exposure to ultraviolet radiation, a change in temperature, or contact with a redox initiator.

The core and reactive groups can also be selected so as to provide a compound that has one of more of the following features: are biocompatible, are non-immunogenic, and do not leave any toxic, inflammatory or immunogenic reaction products at the site of administration. Similarly, the core and reactive groups can also be selected so as to provide a resulting matrix that has one or more of these features.

In one embodiment of the invention, substantially immediately or immediately upon exposure to the modified environment, the self-reactive compounds inter-react form a three-dimensional matrix. The term “substantially immediately” is intended to mean within less than five minutes, preferably within less than two minutes, and the term “immediately” is intended to mean within less than one minute, preferably within less than 30 seconds.

In one embodiment, the self-reactive compound and resulting matrix are not subject to enzymatic cleavage by matrix metalloproteinases such as collagenase, and are therefore not readily degradable in vivo. Further, the self-reactive compound may be readily tailored, in terms of the selection and quantity of each component, to enhance certain properties, e.g., compression strength, swellability, tack, hydrophilicity, optical clarity, and the like.

In one preferred embodiment, R is a hydrophilic polymer. In another preferred embodiment, X is a nucleophilic group, Y is an electrophilic group and Z is either an electrophilic or a nucleophilic group. Additional embodiments are detailed below.

A higher degree of inter-reaction, e.g., crosslinking, may be useful when a less swellable matrix is desired or increased compressive strength is desired. In those embodiments, it may be desirable to have n be an integer from 2-12. In addition, when a plurality of self-reactive compounds are utilized, the compounds may be the same or different.

A. Reactive Groups

Prior to use, the self-reactive compound is stored in an initial environment that insures that the compound remain essentially non-reactive until use. Upon modification of this environment, the compound is rendered reactive and a plurality of compounds will then inter-react to form the desired matrix. The initial environment, as well as the modified environment, is thus determined by the nature of the reactive groups involved.

The number of reactive groups can be the same or different. However, in one embodiment of the invention, the number of reactive groups are approximately equal. As used in this context, the term “approximately” refers to a 2:1 to 1:2 ratio of moles of one reactive group to moles of a different reactive groups. A 1:1:1 molar ratio of reactive groups is generally preferred.

In general, the concentration of the self-reactive compounds in the modified environment, when liquid in nature, will be in the range of about 1 to 50 wt %, generally about 2 to 40 wt %. The preferred concentration of the compound in the liquid will depend on a number of factors, including the type of compound (i.e., type of molecular core and reactive groups), its molecular weight, and the end use of the resulting three-dimensional matrix. For example, use of higher concentrations of the compounds, or using highly functionalized compounds, will result in the formation of a more tightly crosslinked network, producing a stiffer, more robust gel. As such, compositions intended for use in tissue augmentation will generally employ concentrations of self-reactive compounds that fall toward the higher end of the preferred concentration range. Compositions intended for use as bioadhesives or in adhesion prevention do not need to be as firm and may therefore contain lower concentrations of the self-reactive compounds.

1) Electrophilic and Nucleophilic Reactive Groups

In one embodiment of the invention, the reactive groups are electrophilic and nucleophilic groups, which undergo a nucleophilic substitution reaction, a nucleophilic addition reaction, or both. The term “electrophilic” refers to a reactive group that is susceptible to nucleophilic attack, i.e., susceptible to reaction with an incoming nucleophilic group. Electrophilic groups herein are positively charged or electron-deficient, typically electron-deficient. The term “nucleophilic” refers to a reactive group that is electron rich, has an unshared pair of electrons acting as a reactive site, and reacts with a positively charged or electron-deficient site. For such reactive groups, the modification in the initial environment comprises the addition of an aqueous medium and/or a change in pH.

In one embodiment of the invention, X1 (also referred to herein as X) can be a nucleophilic group and X2 (also referred to herein as Y) can be an electrophilic group or vice versa, and X3 (also referred to herein as Z) can be either an electrophilic or a nucleophilic group.

X may be virtually any nucleophilic group, so long as reaction can occur with the electrophilic group Y and also with Z, when Z is electrophilic (ZEL). Analogously, Y may be virtually any electrophilic group, so long as reaction can take place with X and also with Z when Z is nucleophilic (Z_{NU}). The only limitation is a practical one, in that reaction between X and Y, and X and Z_{EL}, or Y and Z_{NU} should be fairly rapid and take place automatically upon admixture with an aqueous medium, without need for heat or potentially toxic or non-biodegradable reaction catalysts or other chemical reagents. It is also preferred although not essential that reaction occur without need for ultraviolet or other radiation. In one embodiment, the reactions between X and Y, and between either X and Z_{EL} or Y and Z_{NU}, are complete in under 60 minutes, preferably under 30 minutes. Most preferably, the reaction occurs in about 5 to 15 minutes or less.

Examples of nucleophilic groups suitable as X or Fn_{NU} include, but are not limited to: —NH₂, —NHR¹, —N(R¹)₂, —SH, —OH, —COOH, —C₆H₄—OH, —H, —PH₂, —PHR¹, —P(R¹)₂, —NH—NH₂, —CO—NH—NH₂, —C₅H₄N, etc. wherein R¹is a hydrocarbyl group and each R1 may be the same or different. R¹is typically alkyl or monocyclic aryl, preferably alkyl, and most preferably lower alkyl. Organometallic moieties are also useful nucleophilic groups for the purposes of the invention, particularly those that act as carbanion donors. Examples of organometallic moieties include: Grignard functionalities —R²MgHal wherein R² is a carbon atom (substituted or unsubstituted), and Hal is halo, typically bromo, iodo or chloro, preferably bromo; and lithium-containing functionalities, typically alkyllithium groups; sodium-containing functionalities.

It will be appreciated by those of ordinary skill in the art that certain nucleophilic groups must be activated with a base so as to be capable of reaction with an electrophilic group. For example, when there are nucleophilic sulfhydryl and hydroxyl groups in the self-reactive compound, the compound must be admixed with an aqueous base in order to remove a proton and provide an —S⁻ or —O⁻ species to enable reaction with the electrophilic group. Unless it is desirable for the base to participate in the reaction, a non-nucleophilic base is preferred. In some embodiments, the base may be present as a component of a buffer solution. Suitable bases and corresponding crosslinking reactions are described herein.

The selection of electrophilic groups provided on the self-reactive compound, must be made so that reaction is possible with the specific nucleophilic groups. Thus, when the X reactive groups are amino groups, the Y and any Z_{EL} groups are selected so as to react with amino groups. Analogously, when the X reactive groups are sulfhydryl moieties, the corresponding electrophilic groups are sulfhydryl-reactive groups, and the like. In general, examples of electrophilic groups suitable as Y or Z_{EL} include, but are not limited to, —CO—Cl, —(CO)—O—(CO)—R (where R is an alkyl group), —CH═CH—CH═O and —CH═CH—C(CH₃)═O, halo, —N═C═O, —N═C═S, —SO₂CH═CH₂, —O(CO)—C═CH₂, —O(CO)—C(CH₃)═CH₂, —S—S—(C₅H₄N), —O(CO)—C(CH₂CH₃)═CH₂, —CH═CH—C═NH, —COOH, —(CO)O—N(COCH₂)₂, —CHO, —(CO)O—N(COCH₂)₂—S(O)₂OH, and —N(COCH)₂.

When X is amino (generally although not necessarily primary amino), the electrophilic groups present on Y and Z_{EL} are amine-reactive groups. Exemplary amine-reactive groups include, by way of example and not limitation, the following groups, or radicals thereof: (1) carboxylic acid esters, including cyclic esters and “activated” esters; (2) acid chloride groups (—CO—Cl); (3) anhydrides (—(CO)—O—(CO)—R, where R is an alkyl group); (4) ketones and aldehydes, including α,β-unsaturated aldehydes and ketones such as —CH═CH—CH═O and —CH═CH—C(CH₃)═O; (5) halo groups; (6) isocyanate group (—N═C═O); (7) thioisocyanato group (—N═C═S); (8) epoxides; (9) activated hydroxyl groups (e.g., activated with conventional activating agents such as carbonyldiimidazole or sulfonyl chloride); and (10) olefins, including conjugated olefins, such as ethenesulfonyl (—SO₂CH═CH₂) and analogous functional groups, including acrylate (—O(CO)—C═CH₂), methacrylate (—O(CO)—C(CH₃)═CH₂), ethyl acrylate (—O(CO)—C(CH₂CH₃)═CH₂), and ethyleneimino (—CH═CH—C═NH).

In one embodiment the amine-reactive groups contain an electrophilically reactive carbonyl group susceptible to nucleophilic attack by a primary or secondary amine, for example the carboxylic acid esters and aldehydes noted above, as well as carboxyl groups (—COOH).

Since a carboxylic acid group per se is not susceptible to reaction with a nucleophilic amine, components containing carboxylic acid groups must be activated so as to be amine-reactive. Activation may be accomplished in a variety of ways, but often involves reaction with a suitable hydroxyl-containing compound in the presence of a dehydrating agent such as dicyclohexylcarbodiimide (DCC) or dicyclohexylurea (DHU). For example, a carboxylic acid can be reacted with an alkoxy-substituted N-hydroxy-succinimide or N-hydroxysulfosuccinimide in the presence of DCC to form reactive electrophilic groups, the N-hydroxysuccinimide ester and the N-hydroxysulfosuccinimide ester, respectively. Carboxylic acids may also be activated by reaction with an acyl halide such as an acyl chloride (e.g., acetyl chloride), to provide a reactive anhydride group. In a further example, a carboxylic acid may be converted to an acid chloride group using, e.g., thionyl chloride or an acyl chloride capable of an exchange reaction. Specific reagents and procedures used to carry out such activation reactions will be known to those of ordinary skill in the art and are described in the pertinent texts and literature.

Accordingly, in one embodiment, the amine-reactive groups are selected from succinimidyl ester (—O(CO)—N(COCH₂)₂), sulfosuccinimidyl ester (—O(CO)—N(COCH₂)₂—S(O)₂OH), maleimido (—N(COCH)₂), epoxy, isocyanato, thioisocyanato, and ethenesulfonyl.

Analogously, when X is sulfhydryl, the electrophilic groups present on Y and Z_{EL} are groups that react with a sulfhydryl moiety. Such reactive groups include those that form thioester linkages upon reaction with a sulfhydryl group, such as those described in WO 00/62827 to Wallace et al. As explained in detail therein, sulfhydryl reactive groups include, but are not limited to: mixed anhydrides; ester derivatives of phosphorus; ester derivatives of p-nitrophenol, p-nitrothiophenol and pentafluorophenol; esters of substituted hydroxylamines, including N-hydroxyphthalimide esters, N-hydroxysuccinimide esters, N-hydroxysulfosuccinimide esters, and N-hydroxyglutarimide esters; esters of 1-hydroxybenzotriazole; 3-hydroxy-3,4-dhydydro-benzotriazin-4-one; 3-hydroxy-3,4-dihydro-quinazoline-4-one; carbonylimidazole derivatives; acid chlorides; ketenes; and isocyanates. With these sulfhydryl reactive groups, auxiliary reagents can also be used to facilitate bond formation, e.g., 1-ethyl-3-[3-dimethylaminopropyl]carbodiimide can be used to facilitate coupling of sulfhydryl groups to carboxyl-containing groups.

In addition to the sulfhydryl reactive groups that form thioester linkages, various other sulfhydryl reactive functionalities can be utilized that form other types of linkages. For example, compounds that contain methyl imidate derivatives form imido-thioester linkages with sulfhydryl groups. Alternatively, sulfhydryl reactive groups can be employed that form disulfide bonds with sulfhydryl groups; such groups generally have the structure —S—S—Ar where Ar is a substituted or unsubstituted nitrogen-containing heteroaromatic moiety or a non-heterocyclic aromatic group substituted with an electron-withdrawing moiety, such that Ar may be, for example, 4-pyridinyl, o-nitrophenyl, m-nitrophenyl, p-nitrophenyl, 2,4-dinitrophenyl, 2-nitro-4-benzoic acid, 2-nitro-4-pyridinyl, etc. In such instances, auxiliary reagents, i.e., mild oxidizing agents such as hydrogen peroxide, can be used to facilitate disulfide bond formation.

Yet another class of sulfhydryl reactive groups forms thioether bonds with sulfhydryl groups. Such groups include, inter alia, maleimido, substituted maleimido, haloalkyl, epoxy, imino, and aziridino, as well as olefins (including conjugated olefins) such as ethenesulfonyl, etheneimino, acrylate, methacrylate, and α,β-unsaturated aldehydes and ketones.

When X is —OH, the electrophilic functional groups on the remaining component(s) must react with hydroxyl groups. The hydroxyl group may be activated as described above with respect to carboxylic acid groups, or it may react directly in the presence of base with a sufficiently reactive electrophilic group such as an epoxide group, an aziridine group, an acyl halide, an anhydride, and so forth.

When X is an organometallic nucleophilic group such as a Grignard functionality or an alkyllithium group, suitable electrophilic functional groups for reaction therewith are those containing carbonyl groups, including, by way of example, ketones and aldehydes.

It will also be appreciated that certain functional groups can react as nucleophilic or as electrophilic groups, depending on the selected reaction partner and/or the reaction conditions. For example, a carboxylic acid group can act as a nucleophilic group in the presence of a fairly strong base, but generally acts as an electrophilic group allowing nucleophilic attack at the carbonyl carbon and concomitant replacement of the hydroxyl group with the incoming nucleophilic group.

These, as well as other embodiments are illustrated below, where the covalent linkages in the matrix that result upon covalent binding of specific nucleophilic reactive groups to specific electrophilic reactive groups on the self-reactive compound include, solely by way of example, the following Table:

TABLE

| Representative | | |
|---|---|---|
| Nucleophilic | Representative Electrophilic | |
| Group (X, Z_{NU}) | Group (Y, Z_{EL}) | Resulting Linkage |
| —NH₂ | —O—(CO)—O—N(COCH₂)₂ | —NH—(CO)—O— |
| | succinimidyl carbonate terminus | |
| —SH | —O—(CO)—O—N(COCH₂)₂ | —S—(CO)—O— |
| —OH | —O—(CO)—O—N(COCH₂)₂ | —O—(CO)— |
| —NH₂ | —O(CO)—CH═CH₂ | —NH—CH₂CH₂—(CO)—O— |
| | acrylate terminus | |
| —SH | —O—(CO)—CH═CH₂ | —S—CH₂CH₂—(CO)—O— |
| —OH | —O—(CO)—CH═CH₂ | —O—CH₂CH₂—(CO)—O— |
| —NH₂ | —O(CO)—(CH₂)₃—CO₂—N(COCH₂)₂ | —NH—(CO)—(CH₂)₃—(CO)—O— |
| | succinimidyl glutarate terminus | |
| —SH | —O(CO)—(CH₂)₃—CO₂—N(COCH₂)₂ | —S—(CO)—(CH₂)₃—(CO)—O— |
| —OH | —O(CO)—(CH₂)₃—CO₂—N(COCH₂)₂ | —O—(CO)—(CH₂)₃—(CO)—O— |
| —NH₂ | —O—CH₂—CO₂—N(COCH₂)₂ | —NH—(CO)—CH₂—O— |
| | succinimidyl acetate terminus | |
| —SH | —O—CH₂—CO₂—N(COCH₂)₂ | —S—(CO)—CH₂—O— |
| —OH | —O—CH₂—CO₂—N(COCH₂)₂ | —O—(CO)—CH₂—O— |
| —NH₂ | —O—NH(CO)—(CH₂)₂—CO₂— | —NH—(CO)—(CH₂)₂—(CO)— |
| | N(COCH₂)₂ | NH—O— |
| | succinimidyl succinamide | |
| | terminus | |
| —SH | —O—NH(CO)—(CH₂)₂—CO₂— | —S—(CO)—(CH₂)₂—(CO)—NH— |
| | N(COCH₂)₂ | O— |
| —OH | —O—NH(CO)—(CH₂)₂—CO₂— | —O—(CO)—(CH₂)₂—(CO)—NH— |
| | N(COCH₂)₂ | O— |
| —NH₂ | —O—(CH₂)₂—CHO | —NH—(CO)—(CH₂)₂—O— |
| | propionaldehyde terminus | |
| —NH₂ | 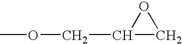 | —NH—CH₂—CH(OH)—CH₂—O—and —N[CH₂—CH(OH)—CH₂—O—]₂ |
| | glycidyl ether terminus | |
| —NH₂ | —O—(CH₂)₂—N═C═O | —NH—(CO)—NH—CH₂—O— |
| | (isocyanate terminus) | |
| —NH₂ | —SO₂—CH═CH₂ | —NH—CH₂CH₂—SO₂— |
| | vinyl sulfone terminus | |
| —SH | —SO₂—CH═CH₂ | —S—CH₂CH₂—SO₂— |

For self-reactive compounds containing electrophilic and nucleophilic reactive groups, the initial environment typically can be dry and sterile. Since electrophilic groups react with water, storage in sterile, dry form will prevent hydrolysis. The dry synthetic polymer may be compression molded into a thin sheet or membrane, which can then be sterilized using gamma or e-beam irradiation. The resulting dry membrane or sheet can be cut to the desired size or chopped into smaller size particulates. The modification of a dry initial environment will typically comprise the addition of an aqueous medium.

In one embodiment, the initial environment can be an aqueous medium such as in a low pH buffer, i.e., having a pH less than about 6.0, in which both electrophilic and nucleophilic groups are non-reactive. Suitable liquid media for storage of such compounds include aqueous buffer solutions such as monobasic sodium phosphate/dibasic sodium phosphate, sodium carbonate/sodium bicarbonate, glutamate or acetate, at a concentration of 0.5 to 300 mM. Modification of an initial low pH aqueous environment will typically comprise increasing the pH to at least pH 7.0, more preferably increasing the pH to at least pH 9.5.

In another embodiment the modification of a dry initial environment comprises dissolving the self-reactive compound in a first buffer solution having a pH within the range of about 1.0 to 5.5 to form a homogeneous solution, and (ii) adding a second buffer solution having a pH within the range of about 6.0 to 11.0 to the homogeneous solution. The buffer solutions are aqueous and can be any pharmaceutically acceptable basic or acid composition. The term “buffer” is used in a general sense to refer to an acidic or basic aqueous solution, where the solution may or may not be functioning to provide a buffering effect (i.e., resistance to change in pH upon addition of acid or base) in the compositions of the present invention. For example, the self-reactive compound can be in the form of a homogeneous dry powder. This powder is then combined with a buffer solution having a pH within the range of about 1.0 to 5.5 to form a homogeneous acidic aqueous solution, and this solution is then combined with a buffer solution having a pH within the range of about 6.0 to 11.0 to form a reactive solution. For example, 0.375 grams of the dry powder can be combined with 0.75 grams of the acid buffer to provide, after mixing, a homogeneous solution, where this solution is combined with 1.1 grams of the basic buffer to provide a reactive mixture that substantially immediately forms a three-dimensional matrix.

Acidic buffer solutions having a pH within the range of about 1.0 to 5.5, include by way of illustration and not limitation, solutions of: citric acid, hydrochloric acid, phosphoric acid, sulfuric acid, AMPSO (3-[(1,1-dimethyl-2-hydroxyethyl)amino]2-hydroxy-propane-sulfonic acid), acetic acid, lactic acid, and combinations thereof. In a preferred embodiment, the acidic buffer solution, is a solution of citric acid, hydrochloric acid, phosphoric acid, sulfuric acid, and combinations thereof. Regardless of the precise acidifying agent, the acidic buffer preferably has a pH such that it retards the reactivity of the nucleophilic groups on the core. For example, a pH of 2.1 is generally sufficient to retard the nucleophilicity of thiol groups. A lower pH is typically preferred when the core contains amine groups as the nucleophilic groups. In general, the acidic buffer is an acidic solution that, when contacted with nucleophilic groups, renders those nucleophilic groups relatively non-nucleophilic.

An exemplary acidic buffer is a solution of hydrochloric acid, having a concentration of about 6.3 mM and a pH in the range of 2.1 to 2.3. This buffer may be prepared by combining concentrated hydrochloric acid with water, i.e., by diluting concentrated hydrochloric acid with water. Similarly, this buffer A may also be conveniently prepared by diluting 1.23 grams of concentrated hydrochloric acid to a volume of 2 liters, or diluting 1.84 grams of concentrated hydrochloric acid to a volume to 3 liters, or diluting 2.45 grams of concentrated hydrochloric acid to a volume of 4 liters, or diluting 3.07 grams concentrated hydrochloric acid to a volume of 5 liters, or diluting 3.68 grams of concentrated hydrochloric acid to a volume to 6 liters. For safety reasons, the concentrated acid is preferably added to water.

Basic buffer solutions having a pH within the range of about 6.0 to 11.0, include by way of illustration and not limitation, solutions of: glutamate, acetate, carbonate and carbonate salts (e.g., sodium carbonate, sodium carbonate monohydrate and sodium bicarbonate), borate, phosphate and phosphate salts (e.g., monobasic sodium phosphate monohydrate and dibasic sodium phosphate), and combinations thereof. In a preferred embodiment, the basic buffer solution is a solution of carbonate salts, phosphate salts, and combinations thereof.

In general, the basic buffer is an aqueous solution that neutralizes the effect of the acidic buffer, when it is added to the homogeneous solution of the compound and first buffer, so that the nucleophilic groups on the core regain their nucleophilic character (that has been masked by the action of the acidic buffer), thus allowing the nucleophilic groups to inter-react with the electrophilic groups on the core.

An exemplary basic buffer is an aqueous solution of carbonate and phosphate salts. This buffer may be prepared by combining a base solution with a salt solution. The salt solution may be prepared by combining 34.7 g of monobasic sodium phosphate monohydrate, 49.3 g of sodium carbonate monohydrate, and sufficient water to provide a solution volume of 2 liter. Similarly, a 6 liter solution may be prepared by combining 104.0 g of monobasic sodium phosphate monohydrate, 147.94 g of sodium carbonate monohydrate, and sufficient water to provide 6 liter of the salt solution. The basic buffer may be prepared by combining 7.2 g of sodium hydroxide with 180.0 g of water. The basic buffer is typically prepared by adding the base solution as needed to the salt solution, ultimately to provide a mixture having the desired pH, e.g., a pH of 9.65 to 9.75.

In general, the basic species present in the basic buffer should be sufficiently basic to neutralize the acidity provided by the acidic buffer, but should not be so nucleophilic itself that it will react substantially with the electrophilic groups on the core. For this reason, relatively “soft” bases such as carbonate and phosphate are preferred in this embodiment of the invention.

To illustrate the preparation of a three-dimensional matrix of the present invention, one may combine an admixture of the self-reactive compound with a first, acidic, buffer (e.g., an acid solution, e.g., a dilute hydrochloric acid solution) to form a homogeneous solution. This homogeneous solution is mixed with a second, basic, buffer (e.g., a basic solution, e.g., an aqueous solution containing phosphate and carbonate salts) whereupon the reactive groups on the core of the self-reactive compound substantially immediately inter-react with one another to form a three-dimensional matrix.

2) Redox Reactive Groups

In one embodiment of the invention, the reactive groups are vinyl groups such as styrene derivatives, which undergo a radical polymerization upon initiation with a redox initiator. The term “redox” refers to a reactive group that is susceptible to oxidation-reduction activation. The term “vinyl” refers to a reactive group that is activated by a redox initiator, and forms a radical upon reaction. X, Y and Z can be the same or different vinyl groups, for example, methacrylic groups.

For self-reactive compounds containing vinyl reactive groups, the initial environment typically will be an aqueous environment. The modification of the initial environment involves the addition of a redox initiator.

3) Oxidative Coupling Reactive Groups

In one embodiment of the invention, the reactive groups undergo an oxidative coupling reaction. For example, X, Y and Z can be a halo group such as chloro, with an adjacent electron-withdrawing group on the halogen-bearing carbon (e.g., on the “L” linking group). Exemplary electron-withdrawing groups include nitro, aryl, and so forth.

For such reactive groups, the modification in the initial environment comprises a change in pH. For example, in the presence of a base such as KOH, the self-reactive compounds then undergo a de-hydro, chloro coupling reaction, forming a double bond between the carbon atoms, as illustrated below:

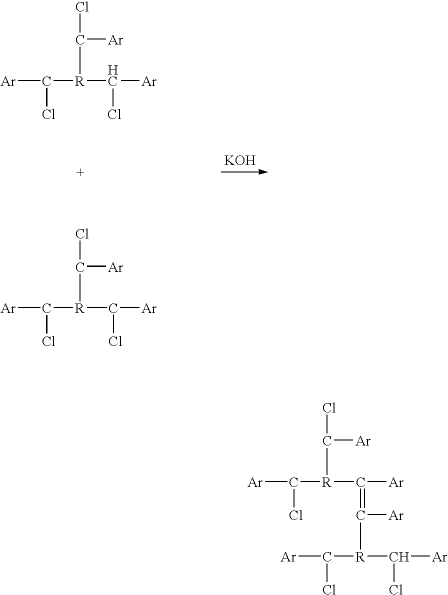

For self-reactive compounds containing oxidative coupling reactive groups, the initial environment typically can be can be dry and sterile, or a non-basic medium. The modification of the initial environment will typically comprise the addition of a base.

4) Photoinitiated Reactive Groups

In one embodiment of the invention, the reactive groups are photoinitiated groups. For such reactive groups, the modification in the initial environment comprises exposure to ultraviolet radiation.

In one embodiment of the invention, X can be an azide (—N₃) group and Y can be an alkyl group such as —CH(CH₃)₂ or vice versa. Exposure to ultraviolet radiation will then form a bond between the groups to provide for the following linkage: —NH—C(CH₃)₂—CH₂—. In another embodiment of the invention, X can be a benzophenone (—(C₆H₄)—C(O)—(C₆H₅)) group and Y can be an alkyl group such as —CH(CH₃)₂ or vice versa. Exposure to ultraviolet radiation will then form a bond between the groups to provide for the following linkage:

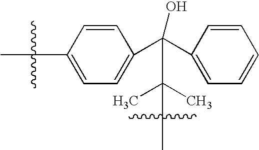

For self-reactive compounds containing photoinitiated reactive groups, the initial environment typically will be in an ultraviolet radiation-shielded environment. This can be for example, storage within a container that is impermeable to ultraviolet radiation.

The modification of the initial environment will typically comprise exposure to ultraviolet radiation.

5) Temperature-Sensitive Reactive Groups

In one embodiment of the invention, the reactive groups are temperature-sensitive groups, which undergo a thermochemical reaction. For such reactive groups, the modification in the initial environment thus comprises a change in temperature. The term “temperature-sensitive” refers to a reactive group that is chemically inert at one temperature or temperature range and reactive at a different temperature or temperature range.

In one embodiment of the invention, X, Y, and Z are the same or different vinyl groups.

For self-reactive compounds containing reactive groups that are temperature-sensitive, the initial environment typically will be within the range of about 10 to 30° C.

The modification of the initial environment will typically comprise changing the temperature to within the range of about 20 to 40° C.

B. Linking Groups

The reactive groups may be directly attached to the core, or they may be indirectly attached through a linking group, with longer linking groups also termed “chain extenders.” In the formula (I) shown above, the optional linker groups are represented by L¹, L², and L³, wherein the linking groups are present when p, q and r are equal to 1.

Suitable linking groups are well known in the art. See, for example, WO 97/22371 to Rhee et al. Linking groups are useful to avoid steric hindrance problems that can sometimes associated with the formation of direct linkages between molecules. Linking groups may additionally be used to link several self-reactive compounds together to make larger molecules. In one embodiment, a linking group can be used to alter the degradative properties of the compositions after administration and resultant gel formation. For example, linking groups can be used to promote hydrolysis, to discourage hydrolysis, or to provide a site for enzymatic degradation.

Examples of linking groups that provide hydrolyzable sites, include, inter alia: ester linkages; anhydride linkages, such as those obtained by incorporation of glutarate and succinate; ortho ester linkages; ortho carbonate linkages such as trimethylene carbonate; amide linkages; phosphoester linkages; α-hydroxy acid linkages, such as those obtained by incorporation of lactic acid and glycolic acid; lactone-based linkages, such as those obtained by incorporation of caprolactone, valerolactone, γ-butyrolactone and p-dioxanone; and amide linkages such as in a dimeric, oligomeric, or poly(amino acid) segment. Examples of non-degradable linking groups include succinimide, propionic acid and carboxymethylate linkages. See, for example, WO 99/07417 to Coury et al. Examples of enzymatically degradable linkages include Leu-Gly-Pro-Ala, which is degraded by collagenase; and Gly-Pro-Lys, which is degraded by plasmin.

Linking groups can also be included to enhance or suppress the reactivity of the various reactive groups. For example, electron-withdrawing groups within one or two carbons of a sulfhydryl group would be expected to diminish its effectiveness in coupling, due to a lowering of nucleophilicity. Carbon-carbon double bonds and carbonyl groups will also have such an effect. Conversely, electron-withdrawing groups adjacent to a carbonyl group (e.g., the reactive carbonyl of glutaryl-N-hydroxysuccinimidyl) would increase the reactivity of the carbonyl carbon with respect to an incoming nucleophilic group. By contrast, sterically bulky groups in the vicinity of a reactive group can be used to diminish reactivity and thus reduce the coupling rate as a result of steric hindrance.

By way of example, particular linking groups and corresponding formulas are indicated in the following Table:

TABLE

| Linking group | Component structure |
|---|---|
| —O—(CH₂)ₓ— | —O—(CH₂)ₓ—X |
| | —O—(CH₂)ₓ—Y |
| | —O—(CH₂)ₓ—Z |
| —S—(CH₂)ₓ— | —S—(CH₂)ₓ—X |
| | —S—(CH₂)ₓ—Y |
| | —S—(CH₂)ₓ—Z |
| —NH—(CH₂)ₓ— | —NH—(CH₂)ₓ—X |
| | —NH—(CH₂)ₓ—Y |
| | —NH—(CH₂)ₓ—Z |
| —O—(CO)—NH—(CH₂)ₓ— | —O—(CO)—NH—(CH₂)ₓ—X |
| | —O—(CO)—NH—(CH₂)ₓ—Y |
| | —O—(CO)—NH—(CH₂)ₓ—Z |
| —NH—(CO)—O—CH₂)ₓ— | —NH—(CO)—O—CH₂)ₓ—X |
| | —NH—(CO)—O—CH₂)ₓ—Y |
| | —NH—(CO)—O—CH₂)ₓ—Z |
| —O—(CO)—(CH₂)ₓ— | —O—(CO)—(CH₂)ₓ—X |
| | —O—(CO)—(CH₂)ₓ—Y |
| | —O—(CO)—(CH₂)ₓ—Z |
| —(CO)—O—(CH₂)ₓ— | —(CO)—O—(CH₂)ₙ—X |
| | —(CO)—O—(CH₂)ₙ—Y |
| | —(CO)—O—(CH₂)ₙ—Z |
| —O—(CO)—O—(CH₂)ₓ— | —O—(CO)—O—(CH₂)ₓ—X |
| | —O—(CO)—O—(CH₂)ₓ—Y |
| | —O—(CO)—O—(CH₂)ₓ—Z |
| —O—(CO)—CHR²— | —O—(CO)—CHR²—X |
| | —O—(CO)—CHR²—Y |
| | —O—(CO)—CHR²—Z |
| —O—R³—(CO)—NH— | —O—R³—(CO)—NH—X |
| | —O—R³—(CO)—NH—Y |
| | —O—R³—(CO)—NH—Z |

In the above Table, x is generally in the range of 1 to about 10; R² is generally hydrocarbyl, typically alkyl or aryl, preferably alkyl, and most preferably lower alkyl; and R³ is hydrocarbylene, heteroatom-containing hydrocarbylene, substituted hydrocarbylene, or substituted heteroatom-containing hydrocarbylene) typically alkylene or arylene (again, optionally substituted and/or containing a heteroatom), preferably lower alkylene (e.g., methylene, ethylene, n-propylene, n-butylene, etc.), phenylene, or amidoalkylene (e.g., —(CO)—NH—CH₂).

Other general principles that should be considered with respect to linking groups are as follows. If a higher molecular weight self-reactive compound is to be used, it will preferably have biodegradable linkages as described above, so that fragments larger than 20,000 mol. wt. are not generated during resorption in the body. In addition, to promote water miscibility and/or solubility, it may be desired to add sufficient electric charge or hydrophilicity. Hydrophilic groups can be easily introduced using known chemical synthesis, so long as they do not give rise to unwanted swelling or an undesirable decrease in compressive strength. In particular, polyalkoxy segments may weaken gel strength.

C. The Core

The “core” of each self-reactive compound is comprised of the molecular structure to which the reactive groups are bound. The molecular core can a polymer, which includes synthetic polymers and naturally occurring polymers. In one embodiment, the core is a polymer containing repeating monomer units. The polymers can be hydrophilic, hydrophobic, or amphiphilic. The molecular core can also be a low molecular weight components such as a C₂₋₁₄ hydrocarbyl or a heteroatom-containing C₂₋₁₄ hydrocarbyl. The heteroatom-containing C₂₋₁₄ hydrocarbyl can have 1 or 2 heteroatoms selected from N, O and S. In a preferred embodiment, the self-reactive compound comprises a molecular core of a synthetic hydrophilic polymer.

1) Hydrophilic Polymers

As mentioned above, the term “hydrophilic polymer” as used herein refers to a polymer having an average molecular weight and composition that naturally renders, or is selected to render the polymer as a whole “hydrophilic.” Preferred polymers are highly pure or are purified to a highly pure state such that the polymer is or is treated to become pharmaceutically pure. Most hydrophilic polymers can be rendered water soluble by incorporating a sufficient number of oxygen (or less frequently nitrogen) atoms available for forming hydrogen bonds in aqueous solutions.

Synthetic hydrophilic polymers may be homopolymers, block copolymers including di-block and tri-block copolymers, random copolymers, or graft copolymers. In addition, the polymer may be linear or branched, and if branched, may be minimally to highly branched, dendrimeric, hyperbranched, or a star polymer. The polymer may include biodegradable segments and blocks, either distributed throughout the polymer's molecular structure or present as a single block, as in a block copolymer. Biodegradable segments preferably degrade so as to break covalent bonds. Typically, biodegradable segments are segments that are hydrolyzed in the presence of water and/or enzymatically cleaved in situ. Biodegradable segments may be composed of small molecular segments such as ester linkages, anhydride linkages, ortho ester linkages, ortho carbonate linkages, amide linkages, phosphonate linkages, etc. Larger biodegradable “blocks” will generally be composed of oligomeric or polymeric segments incorporated within the hydrophilic polymer. Illustrative oligomeric and polymeric segments that are biodegradable include, by way of example, poly(amino acid) segments, poly(orthoester) segments, poly(orthocarbonate) segments, and the like. Other biodegradable segments that may form part of the hydrophilic polymer core include polyesters such as polylactide, polyethers such as polyalkylene oxide, polyamides such as a protein, and polyurethanes. For example, the core of the self-reactive compound can be a diblock copolymer of tetrafunctionally activated polyethylene glycol and polylactide.

Synthetic hydrophilic polymers that are useful herein include, but are not limited to: polyalkylene oxides, particularly polyethylene glycol (PEG) and poly(ethylene oxide)-poly(propylene oxide) copolymers, including block and random copolymers; polyols such as glycerol, polyglycerol (PG) and particularly highly branched polyglycerol, propylene glycol; poly(oxyalkylene)-substituted diols, and poly(oxyalkylene)-substituted polyols such as mono-, di- and tri-polyoxyethylated glycerol, mono- and di-polyoxyethylated propylene glycol, and mono- and di-polyoxyethylated trimethylene glycol; polyoxyethylated sorbitol, polyoxyethylated glucose; poly(acrylic acids) and analogs and copolymers thereof, such as polyacrylic acid per se, polymethacrylic acid, poly(hydroxyethylmethacrylate), poly(hydroxyethylacrylate), poly(methylalkylsulfoxide methacrylates), poly(methylalkylsulfoxide acrylates) and copolymers of any of the foregoing, and/or with additional acrylate species such as aminoethyl acrylate and mono-2-(acryloxy)-ethyl succinate; polymaleic acid; poly(acrylamides) such as polyacrylamide per se, poly(methacrylamide), poly(dimethylacrylamide), poly(N-isopropyl-acrylamide), and copolymers thereof; poly(olefinic alcohols) such as poly(vinyl alcohols) and copolymers thereof; poly(N-vinyl lactams) such as poly(vinyl pyrrolidones), poly(N-vinyl caprolactams), and copolymers thereof; polyoxazolines, including poly(methyloxazoline) and poly(ethyloxazoline); and polyvinylamines; as well as copolymers of any of the foregoing. It must be emphasized that the aforementioned list of polymers is not exhaustive, and a variety of other synthetic hydrophilic polymers may be used, as will be appreciated by those skilled in the art.

Those of ordinary skill in the art will appreciate that synthetic polymers such as polyethylene glycol cannot be prepared practically to have exact molecular weights, and that the term “molecular weight” as used herein refers to the weight average molecular weight of a number of molecules in any given sample, as commonly used in the art. Thus, a sample of PEG 2,000 might contain a statistical mixture of polymer molecules ranging in weight from, for example, 1,500 to 2,500 daltons with one molecule differing slightly from the next over a range. Specification of a range of molecular weights indicates that the average molecular weight may be any value between the limits specified, and may include molecules outside those limits. Thus, a molecular weight range of about 800 to about 20,000 indicates an average molecular weight of at least about 800, ranging up to about 20 kDa.

Other suitable synthetic hydrophilic polymers include chemically synthesized polypeptides, particularly polynucleophilic polypeptides that have been synthesized to incorporate amino acids containing primary amino groups (such as lysine) and/or amino acids containing thiol groups (such as cysteine). Poly(lysine), a synthetically produced polymer of the amino acid lysine (145 MW), is particularly preferred. Poly(lysine)s have been prepared having anywhere from 6 to about 4,000 primary amino groups, corresponding to molecular weights of about 870 to about 580,000. Poly(lysine)s for use in the present invention preferably have a molecular weight within the range of about 1,000 to about 300,000, more preferably within the range of about 5,000 to about 100,000, and most preferably, within the range of about 8,000 to about 15,000. Poly(lysine)s of varying molecular weights are commercially available from Peninsula Laboratories, Inc. (Belmont, Calif.).

Although a variety of different synthetic hydrophilic polymers can be used in the present compounds, preferred synthetic hydrophilic polymers are PEG and PG, particularly highly branched PG. Various forms of PEG are extensively used in the modification of biologically active molecules because PEG lacks toxicity, antigenicity, and immunogenicity (i.e., is biocompatible), can be formulated so as to have a wide range of solubilities, and does not typically interfere with the enzymatic activities and/or conformations of peptides. A particularly preferred synthetic hydrophilic polymer for certain applications is a PEG having a molecular weight within the range of about 100 to about 100,000, although for highly branched PEG, far higher molecular weight polymers can be employed, up to 1,000,000 or more, providing that biodegradable sites are incorporated ensuring that all degradation products will have a molecular weight of less than about 30,000. For most PEGs, however, the preferred molecular weight is about 1,000 to about 20,000, more preferably within the range of about 7,500 to about 20,000. Most preferably, the polyethylene glycol has a molecular weight of approximately 10,000.

Naturally occurring hydrophilic polymers include, but are not limited to: proteins such as collagen, fibronectin, albumins, globulins, fibrinogen, fibrin and thrombin, with collagen particularly preferred; carboxylated polysaccharides such as polymannuronic acid and polygalacturonic acid; aminated polysaccharides, particularly the glycosaminoglycans, e.g., hyaluronic acid, chitin, chondroitin sulfate A, B, or C, keratin sulfate, keratosulfate and heparin; and activated polysaccharides such as dextran and starch derivatives. Collagen and glycosaminoglycans are preferred naturally occurring hydrophilic polymers for use herein.

Unless otherwise specified, the term “collagen” as used herein refers to all forms of collagen, including those, which have been processed or otherwise modified. Thus, collagen from any source may be used in the compounds of the invention; for example, collagen may be extracted and purified from human or other mammalian source, such as bovine or porcine corium and human placenta, or may be recombinantly or otherwise produced. The preparation of purified, substantially non-antigenic collagen in solution from bovine skin is well known in the art. For example, U.S. Pat. No. 5,428,022 to Palefsky et al. discloses methods of extracting and purifying collagen from the human placenta, and U.S. Pat. No. 5,667,839 to Berg discloses methods of producing recombinant human collagen in the milk of transgenic animals, including transgenic cows. Non-transgenic, recombinant collagen expression in yeast and other cell lines) is described in U.S. Pat. No. 6,413,742 to Olsen et al., U.S Pat. No. 6,428,978 to Olsen et al., and U.S. Pat. No. 6,653,450 to Berg et al.

Collagen of any type, including, but not limited to, types I, II, III, IV, or any combination thereof, may be used in the compounds of the invention, although type I is generally preferred. Either atelopeptide or telopeptide-containing collagen may be used; however, when collagen from a natural source, such as bovine collagen, is used, atelopeptide collagen is generally preferred, because of its reduced immunogenicity compared to telopeptide-containing collagen.

Collagen that has not been previously crosslinked by methods such as heat, irradiation, or chemical crosslinking agents is preferred for use in the invention, although previously crosslinked collagen may be used.

Collagens for use in the present invention are generally, although not necessarily, in aqueous suspension at a concentration between about 20 mg/ml to about 120 mg/ml, preferably between about 30 mg/ml to about 90 mg/ml. Although intact collagen is preferred, denatured collagen, commonly known as gelatin, can also be used. Gelatin may have the added benefit of being degradable faster than collagen.

Nonfibrillar collagen is generally preferred for use in compounds of the invention, although fibrillar collagens may also be used. The term “nonfibrillar collagen” refers to any modified or unmodified collagen material that is in substantially nonfibrillar form, i.e., molecular collagen that is not tightly associated with other collagen molecules so as to form fibers. Typically, a solution of nonfibrillar collagen is more transparent than is a solution of fibrillar collagen. Collagen types that are nonfibrillar (or microfibrillar) in native form include types IV, VI, and VII.

Chemically modified collagens that are in nonfibrillar form at neutral pH include succinylated collagen and methylated collagen, both of which can be prepared according to the methods described in U.S. Pat. No. 4,164,559 to Miyata et al. Methylated collagen, which contains reactive amine groups, is a preferred nucleophile-containing component in the compositions of the present invention. In another aspect, methylated collagen is a component that is present in addition to first and second components in the matrix-forming reaction of the present invention. Methylated collagen is described in, for example, in U.S. Pat. No. 5,614,587 to Rhee et al.

Collagens for use in the compositions of the present invention may start out in fibrillar form, then can be rendered nonfibrillar by the addition of one or more fiber disassembly agent. The fiber disassembly agent must be present in an amount sufficient to render the collagen substantially nonfibrillar at pH 7, as described above. Fiber disassembly agents for use in the present invention include, without limitation, various biocompatible alcohols, amino acids, inorganic salts, and carbohydrates, with biocompatible alcohols being particularly preferred. Preferred biocompatible alcohols include glycerol and propylene glycol. Non-biocompatible alcohols, such as ethanol, methanol, and isopropanol, are not preferred for use in the present invention, due to their potentially deleterious effects on the body of the patient receiving them. Preferred amino acids include arginine. Preferred inorganic salts include sodium chloride and potassium chloride. Although carbohydrates, such as various sugars including sucrose, may be used in the practice of the present invention, they are not as preferred as other types of fiber disassembly agents because they can have cytotoxic effects in vivo.

Fibrillar collagen is less preferred for use in the compounds of the invention. However, as disclosed in U.S. Pat. No. 5,614,587 to Rhee et al., fibrillar collagen, or mixtures of nonfibrillar and fibrillar collagen, may be preferred for use in compounds intended for long-term persistence in vivo.

2) Hydrophobic Polymers

The core of the self-reactive compound may also comprise a hydrophobic polymer, including low molecular weight polyfunctional species, although for most uses hydrophilic polymers are preferred. Generally, “hydrophobic polymers” herein contain a relatively small proportion of oxygen and/or nitrogen atoms. Preferred hydrophobic polymers for use in the invention generally have a carbon chain that is no longer than about 14 carbons. Polymers having carbon chains substantially longer than 14 carbons generally have very poor solubility in aqueous solutions and, as such, have very long reaction times when mixed with aqueous solutions of synthetic polymers containing, for example, multiple nucleophilic groups. Thus, use of short-chain oligomers can avoid solubility-related problems during reaction. Polylactic acid and polyglycolic acid are examples of two particularly suitable hydrophobic polymers.

3) Amphiphilic Polymers

Generally, amphiphilic polymers have a hydrophilic portion and a hydrophobic (or lipophilic) portion. The hydrophilic portion can be at one end of the core and the hydrophobic portion at the opposite end, or the hydrophilic and hydrophobic portions may be distributed randomly (random copolymer) or in the form of sequences or grafts (block copolymer) to form the amphiphilic polymer core of the self-reactive compound. The hydrophilic and hydrophobic portions may include any of the aforementioned hydrophilic and hydrophobic polymers.

Alternately, the amphiphilic polymer core can be a hydrophilic polymer that has been modified with hydrophobic moieties (e.g., alkylated PEG or a hydrophilic polymer modified with one or more fatty chains ), or a hydrophobic polymer that has been modified with hydrophilic moieties (e.g., “PEGylated” phospholipids such as polyethylene glycolated phospholipids).

4) Low Molecular Weight Components

As indicated above, the molecular core of the self-reactive compound can also be a low molecular weight compound, defined herein as being a C₂₋₁₄ hydrocarbyl or a heteroatom-containing C₂₋₁₄ hydrocarbyl, which contains 1 to 2 heteroatoms selected from N, O, S and combinations thereof. Such a molecular core can be substituted with any of the reactive groups described herein.

Alkanes are suitable C₂₋₁₄ hydrocarbyl molecular cores. Exemplary alkanes, for substituted with a nucleophilic primary amino group and a Y electrophilic group, include, ethyleneamine (H₂N—CH₂CH₂—Y), tetramethyleneamine (H₂N—(CH₄)—Y), pentamethyleneamine (H₂N—(CH₅)—Y), and hexamethyleneamine (H₂N—(CH₆)—Y).

Low molecular weight diols and polyols are also suitable C₂₋₁₄ hydrocarbyls and include trimethylolpropane, di(trimethylol propane), pentaerythritol, and diglycerol. Polyacids are also suitable C₂₋₁₄ hydrocarbyls, and include trimethylolpropane-based tricarboxylic acid, di(trimethylol propane)-based tetracarboxylic acid, heptanedioic acid, octanedioic acid (suberic acid), and hexadecanedioic acid (thapsic acid).

Low molecular weight di- and poly-electrophiles are suitable heteroatom-containing C₂₋₁₄ hydrocarbyl molecular cores. These include, for example, disuccinimidyl suberate (DSS), bis(sulfosuccinimidyl) suberate (BS₃), dithiobis(succinimidylpropionate) (DSP), bis(2-succinimidooxycarbonyloxy) ethyl sulfone (BSOCOES), and 3,3′-dithiobis(sulfosuccinimidylpropionate (DTSPP), and their analogs and derivatives.

In one embodiment of the invention, the self-reactive compound of the invention comprises a low-molecular weight material core, with a plurality of acrylate moieties and a plurality of thiol groups.

D. Preparation

The self-reactive compounds are readily synthesized to contain a hydrophilic, hydrophobic or amphiphilic polymer core or a low molecular weight core, functionalized with the desired functional groups, i.e., nucleophilic and electrophilic groups, which enable crosslinking. For example, preparation of a self-reactive compound having a polyethylene glycol (PEG) core is discussed below. However, it is to be understood that the following discussion is for purposes of illustration and analogous techniques may be employed with other polymers.

With respect to PEG, first of all, various functionalized PEGs have been used effectively in fields such as protein modification (see Abuchowski et al., Enzymes as Drugs, John Wiley & Sons: New York, N.Y. (1981) pp. 367-383; and Dreborg et al. (1990) Crit. Rev. Therap. Drug Carrier Syst. 6:315), peptide chemistry (see Mutter et al., The Peptides, Academic: New York, N.Y. 2:285-332; and Zalipsky et al. (1987) Int. J. Peptide Protein Res. 30:740), and the synthesis of polymeric drugs (see Zalipsky et al. (1983) Eur. Polym. J. 19:1177; and Ouchi et al. (1987) J. Macromol. Sci. Chem. A24:1011).

Functionalized forms of PEG, including multi-functionalized PEG, are commercially available, and are also easily prepared using known methods. For example, see Chapter 22 of Poly(ethylene Glycol) Chemistry: Biotechnical and Biomedical Applications, J. Milton Harris, ed., Plenum Press, NY (1992).

Multi-functionalized forms of PEG are of particular interest and include, PEG succinimidyl glutarate, PEG succinimidyl propionate, succinimidyl butylate, PEG succinimidyl acetate, PEG succinimidyl succinamide, PEG succinimidyl carbonate, PEG propionaldehyde, PEG glycidyl ether, PEG-isocyanate, and PEG-vinylsulfone. Many such forms of PEG are described in U.S. Pat. Nos. 5,328,955 and 6,534,591, both to Rhee et al. Similarly, various forms of multi-amino PEG are commercially available from sources such as PEG Shop, a division of SunBio of South Korea (www.sunbio.com), Nippon Oil and Fats (Yebisu Garden Place Tower, 20-3 Ebisu 4-chome, Shibuya-ku, Tokyo), Nektar Therapeutics (San Carlos, Calif., formerly Shearwater Polymers, Huntsville, Ala.) and from Huntsman's Performance Chemicals Group (Houston, Tex.) under the name Jeffamine® polyoxyalkyleneamines. Multi-amino PEGs useful in the present invention include the Jeffamine diamines (“D” series) and triamines (“T” series), which contain two and three primary amino groups per molecule. Analogous poly(sulfhydryl) PEGs are also available from Nektar Therapeutics, e.g., in the form of pentaerythritol poly(ethylene glycol) ether tetra-sulfhydryl (molecular weight 10,000). These multi-functionalized forms of PEG can then be modified to include the other desired reactive groups.

Reaction with succinimidyl groups to convert terminal hydroxyl groups to reactive esters is one technique for preparing a core with electrophilic groups. This core can then be modified include nucleophilic groups such as primary amines, thiols, and hydroxyl groups. Other agents to convert hydroxyl groups include carbonyldiimidazole and sulfonyl chloride. However, as discussed herein, a wide variety of electrophilic groups may be advantageously employed for reaction with corresponding nucleophilic groups. Examples of such electrophilic groups include acid chloride groups; anhydrides, ketones, aldehydes, isocyanate, isothiocyanate, epoxides, and olefins, including conjugated olefins such as ethenesulfonyl (—SO₂CH═CH₂) and analogous functional groups.

Other in situ Crosslinkinq Materials

Numerous other types of in situ forming materials have been described which may be used in combination with an anti-scarring agent in accordance with the invention. The in situ forming material may be a biocompatible crosslinked polymer that is formed from water soluble precursors having electrophilic and nucleophilic groups capable of reacting and crosslinking in situ (see, e.g., U.S. Pat. No. 6,566,406). The in situ forming material may be hydrogel that may be formed through a combination of physical and chemical crosslinking processes, where physical crosslinking is mediated by one or more natural or synthetic components that stabilize the hydrogel-forming precursor solution at a deposition site for a period of time sufficient for more resilient chemical crosslinks to form (see, e.g., U.S. Pat. No. 6,818,018). The in situ forming material may be formed upon exposure to an aqueous fluid from a physiological environment from dry hydrogel precursors (see, e.g., U.S. Pat. No. 6,703,047). The in situ forming material may be a hydrogel matrix that provides controlled release of relatively low molecular weight therapeutic species by first dispersing or dissolving the therapeutic species within relatively hydrophobic rate modifying agents to form a mixture; the mixture is formed into microparticles that are dispersed within bioabsorbable hydrogels, so as to release the water soluble therapeutic agents in a controlled fashion (see, e.g., U.S. Pat. No. 6,632,457). The in situ forming material may be a multi-component hydrogel system (see, e.g., U.S. Pat. No. 6,379, 373). The in situ forming material may be a multi-arm block copolymer that includes a central core molecule, such as a residue of a polyol, and at least three copolymer arms covalently attached to the central core molecule, each copolymer arm comprising an inner hydrophobic polymer segment covalently attached to the central core molecule and an outer hydrophilic polymer segment covalently attached to the hydrophobic polymer segment, wherein the central core molecule and the hydrophobic polymer segment define a hydrophobic core region (see, e.g., U.S. Pat. No. 6,730,334). The in situ forming material may include a gel-forming macromer that includes at least four polymeric blocks, at least two of which are hydrophobic and at least one of which is hydrophilic, and including a crosslinkable group (see, e.g., U.S. Pat. No. 6,639,014). The in situ forming material may be a water-soluble macromer that includes at least one hydrolysable linkage formed from carbonate or dioxanone groups, at least one water-soluble polymeric block, and at least one polymerizable group (see, e.g., U.S. Pat. No. 6,177,095). The in situ forming material may comprise polyoxyalkylene block copolymers that form weak physical crosslinks to provide gels having a paste-like consistency at physiological temperatures. (see, e.g., U.S. Pat. No. 4,911,926). The in situ forming material may be a thermo-irreversible gel made from polyoxyalkylene polymers and ionic polysaccharides (see, e.g., U.S. Pat. No. 5,126,141). The in situ forming material may be a gel forming composition that includes chitin derivatives (see, e.g., U.S. Pat. No. 5,093,319), chitosan-coagulum (see, e.g., U.S. Pat. No. 4,532,134), or hyaluronic acid (see, e.g., U.S. Pat. No. 4,141,973). The in situ forming material may be an in situ modification of alginate (see, e.g., U.S. Pat. No. 5,266,326 ). The in situ forming material may be formed from ethylenically unsaturated water soluble macromers that can be crosslinked in contact with tissues, cells, and bioactive molecules to form gels (see, e.g., U.S. Pat. No. 5,573,934). The in situ forming material may include urethane prepolymers used in combination with an unsaturated cyano compound containing a cyano group attached to a carbon atom, such as cyano(meth)acrylic acids and esters thereof (see, e.g., U.S. Pat. No. 4,740,534). The in situ forming material may be a biodegradable hydrogel that polymerizes by a photoinitiated free radical polymerization from water soluble macromers (see, e.g., U.S. Pat. No. 5,410,016). The in situ forming material may be formed from a two component mixture including a first part comprising a serum albumin protein in an aqueous buffer having a pH in a range of about 8.0-11.0, and a second part comprising a water-compatible or water-soluble bifunctional crosslinking agent. (see, e.g., U.S. Pat. No. 5,583,114).

In another aspect, in situ forming materials that can be used include those based on the crosslinking of proteins. For example, the in situ forming material may be a biodegradable hydrogel composed of a recombinant or natural human serum albumin and poly(ethylene) glycol polymer solution whereby upon mixing the solution cross-links to form a mechanical non-liquid covering structure which acts as a sealant. See e.g., U.S. Pat. Nos. 6,458,147 and 6,371,975. The in situ forming material may be composed of two separate mixtures based on fibrinogen and thrombin which are dispensed together to form a biological adhesive when intermixed either prior to or on the application site to form a fibrin sealant. See e.g., U.S. Pat. No. 6,764,467. The in situ forming material may be composed of ultrasonically treated collagen and albumin which form a viscous material that develops adhesive properties when crosslinked chemically with glutaraldehyde and amino acids or peptides. See e.g., U.S. Pat. No. 6,310,036. The in situ forming material may be a hydrated adhesive gel composed of an aqueous solution consisting essentially of a protein having amino groups at the side chains (e.g., gelatin, albumin) which is crosslinked with an N-hydroxyimidoester compound. See e.g., U.S. Pat. No. 4,839,345. The in situ forming material may be a hydrogel prepared from a protein or polysaccharide backbone (e.g., albumin or polymannuronic acid) bonded to a cross-linking agent (e.g., polyvalent derivatives of polyethylene or polyalkylene glycol). See e.g., U.S. Pat. No. 5,514,379. The in situ forming material may be composed of a polymerizable collagen composition that is applied to the tissue and then exposed to an initiator to polymerize the collagen to form a seal over a wound opening in the tissue. See e.g., U.S. Pat. No. 5,874,537. The in situ forming material may be a two component mixture composed of a protein (e.g., serum albumin) in an aqueous buffer having a pH in the range of about 8.0-11.0 and a water-soluble bifunctional polyethylene oxide type crosslinking agent, which transforms from a liquid to a strong, flexible bonding composition to seal tissue in situ. See e.g., U.S. Pat. Nos. 5,583,114 and RE38158 and PCT Publication No. WO 96/03159. The in situ forming material may be composed of a protein, a surfactant, and a lipid in a liquid carrier, which is crosslinked by adding a crosslinker and used as a sealant or bonding agent in situ. See e.g., U.S. patent application No. 2004/0063613A1 and PCT Publication Nos. WO 01/45761 and WO 03/090683. The in situ forming material may be composed of two enzyme-free liquid components that are mixed by dispensing the components into a catheter tube deployed at the vascular puncture site, wherein, upon mixing, the two liquid components chemically cross-link to form a mechanical non-liquid matrix that seals a vascular puncture site. See e.g., U.S. patent application Nos. 2002/0161399A1 and 2001/0018598A1. The in situ forming material may be a cross-linked albumin composition composed of an albumin preparation and a carbodiimide preparation which are mixed under conditions that permit crosslinking of the albumin for use as a bioadhesive or sealant. See e.g., PCT Publication No. WO 99/66964. The in situ forming material may be composed of collagen and a peroxidase and hydrogen peroxide, such that the collagen is crosslinked to from a semi-solid gel that seals a wound. See e.g., PCT Publication No. WO 01/35882.

In another aspect, in situ forming materials that can be used include those based on isocyanate or isothiocyanate capped polymers. For example, the in situ forming material may be composed of isocyanate-capped polymers that are liquid compositions which form into a solid adhesive coating by in situ polymerization and crosslinking upon contact with body fluid or tissue. See e.g., PCT Publication No. WO 04/021983. The in situ forming material may be a moisture-curing sealant composition composed of an active isocyanato-terminated isocyanate prepolymer containing a polyol component with a molecular weight of 2,000 to 20,000 and an isocyanurating catalyst agent. See e.g., U.S. Pat. No. 5,206,331.

In another embodiment, the reagents can undergo an electrophilic-nucleophilic reaction to produce a crosslinked matrix. Polymers containing and/or terminated with nucleophilic groups such as amine, sulfhydryl, hydroxyl, —PH₂ or CO—NH—NH₂ can be used as the nucleophilic reagents and polymers containing and/or terminated with electrophilic groups such as succinimidyl, carboxylic acid, aldehyde, epoxide, isocyanate, vinyl, vinyl sulfone, maleimide, —S—S—(C₅H₄N) or activated esters, such as are used in peptide synthesis can be used as the electrophilic reagents. For example, a 4-armed thiol derivatized poly(ethylene glycol) (e.g., pentaerythritol poly(ethylene glycol)ether tetra-sulfhydryl) can be reacted with a 4 armed NHS-derivatized polyethylene glycol (e.g., pentaerythritol poly(ethylene glycol)ether tetra-succinimidyl glutarate) under basic conditions (pH>about 8). Representative examples of compositions that undergo such electrophilic-nucleophilic crosslinking reactions are described, for example, in U.S. Pat. Nos. 5,752,974; 5,807,581; 5,874,500; 5,936,035; 6,051,648; 6,165,489; 6,312,725; 6,458,889; 6,495,127; 6,534,591; 6,624,245; 6,566,406; 6,610,033; 6,632,457; and PCT Application Publication Nos. WO 04/060405 and WO 04/060346.

In another embodiment, the electrophilic- or nucleophilic-terminated polymers can further comprise a polymer that can enhance the mechanical and/or adhesive properties of the in situ forming compositions. This polymer can be a degradable or non-degradable polymer. For example, the polymer may be collagen or a collagen derivative, for example methylated collagen. An example of an in situ forming composition uses pentaerythritol poly(ethylene glycol)ether tetra-sulfhydryl) (4-armed thiol PEG), pentaerythritol poly(ethylene glycol)ether tetra-succinimidyl glutarate) (4-armed NHS PEG) and methylated collagen as the reactive reagents. This composition, when mixed with the appropriate buffers can produce a crosslinked hydrogel. (See, e.g., U.S. Pat. Nos. 5,874,500; 6,051,648; 6,166,130; 5,565,519 and 6,312,725).

In another embodiment, the reagents that can form a covalent bond with the tissue to which it is applied may be used. Polymers containing and/or terminated with electrophilic groups such as succinimidyl, aldehyde, epoxide, isocyanate, vinyl, vinyl sulfone, maleimide, —S—S—(C₅H₄N) or activated esters, such as are used in peptide synthesis may be used as the reagents. For example, a 4 armed NHS-derivatized polyethylene glycol (e.g., pentaerythritol poly(ethylene glycol)ether tetra-succinimidyl glutarate) may be applied to the tissue in the solid form or in a solution form. In the preferred embodiment, the 4 armed NHS-derivatized polyethylene glycol is applied to the tissue under basic conditions (pH>about 8). Other representative examples of compositions of this nature that may be used are disclosed in PCT Application Publication No. WO 04/060405 and WO 04/060346, and U.S. patent application No.10/749,123.

In another embodiment, the in situ forming material polymer can be a polyester. Polyesters that can be used in in situ forming compositions include poly(hydroxyesters). In another embodiment, the polyester can comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one. Representative examples of these types of compositions are described in U.S. Pat. Nos. 5,874,500; 5,936,035; 6,312,725; 6,495,127 and PCT Publication Nos. WO 2004/028547.

In another embodiment, the electrophilic-terminated polymer can be partially or completely replaced by a small molecule or oligomer that comprises an electrophilic group (e.g., disuccinimidyl glutarate).

In another embodiment, the nucleophilic-terminated polymer can be partially or completely replaced by a small molecule or oligomer that comprises a nucleophilic group (e.g., dicysteine, dilysine, trilysine, etc.).

Other examples of in situ forming materials that can be used include those based on the crosslinking of proteins (described in, for example, U.S. Pat. Nos. RE38158; 4,839,345; 5,514,379, 5,583,114; 6,310,036; 6,458,147; 6,371,975; U.S. patent application Publication Nos. 2004/0063613A1, 2002/0161399A1, and 2001/0018598A1, and PCT Publication Nos. WO 03/090683, WO 01/45761, WO 99/66964, and WO 96/03159) and those based on isocyanate or isothiocyanate capped polymers (see, e.g., PCT Publication No. WO 04/021983).

Other examples of in situ forming materials can include reagents that comprise one or more cyanoacrylate groups. These reagents can be used to prepare a poly(alkylcyanoacrylate) or poly(carboxyalkylcyanoacrylate) (e.g., poly(ethylcyanoacrylate), poly(butylcyanoacrylate), poly(isobutylcyanoacrylate), poly(hexylcyanoacrylate), poly(methoxypropylcyanoacrylate), and poly(octylcyanoacrylate)).

Examples of commercially available cyanoacrylates that can be used in the present invention include DERMABOND, INDERMIL, GLUSTITCH, VETBOND, HISTOACRYL, TISSUMEND, HISTOACRYL BLUE and ORABASE SOOTHE-N-SEAL LIQUID PROTECTANT.

In another embodiment, the cyanoacrylate compositions may further comprise additives to stabilize the reagents and/or alter the rate of reaction of the cyanoacrylate, and/or plasticize the poly(cyanoacrylate), and/or alter the rate of degradation of the poly(cyanoacrylate). For example, a trimethylene carbonate based polymer or an oxalate polymer of poly(ethylene glycol) or a ε-caprolactone based copolymer may be mixed with a 2-alkoxyalkylcyanoacrylate (e.g., 2-methoxypropylcyanoacrylate). Representative examples of these compositions are described in U.S. Pat. Nos. 5,350,798 and 6,299,631.

In another embodiment, the cyanoacrylate composition can be prepared by capping heterochain polymers with a cyanoacrylate group. The cyanoacrylate-capped heterochain polymer preferably has at least two cyanoacrylate ester groups per chain. The heterochain polymer can comprise an absorbable poly(ester), poly(ester-carbonate), poly(ether-carbonate) and poly(ether-ester). The poly(ether-ester)s described in U.S. Pat. Nos. 5,653,992 and 5,714,159 can also be used as the heterochain polymers. A triaxial poly(ε-caprolactone-co-trimethylene carbonate) is an example of a poly(ester-carbonate) that can be used. The heterochain polymer may be a polyether. Examples of polyethers that can be used include poly(ethylene glycol), poly(propylene glycol) and block copolymers of poly(ethylene glycol) and poly(propylene glycol) (e.g., PLURONICS group of polymers including but not limited to PLURONIC F127 or F68). Representative examples of these compositions are described in U.S. Pat. No. 6,699,940.

Within another aspect of the invention, the biologically active ant-infective and/or fibrosis-inhibiting agent can be delivered with a non-polymeric compound (e.g., a carrier). These non-polymeric carriers can include sucrose derivatives (e.g., sucrose acetate isobutyrate, sucrose oleate), sterols such as cholesterol, stigmasterol, β-sitosterol, and estradiol; cholesteryl esters such as cholesteryl stearate; C₁₂-C₂₄ fatty acids such as lauric acid, myristic acid, palmitic acid, stearic acid, arachidic acid, behenic acid, and lignoceric acid; C₁₈-C₃₆ mono-, di- and triacylglycerides such as glyceryl monooleate, glyceryl monolinoleate, glyceryl monolaurate, glyceryl monodocosanoate, glyceryl monomyristate, glyceryl monodicenoate, glyceryl dipalmitate, glyceryl didocosanoate, glyceryl dimyristate, glyceryl didecenoate, glyceryl tridocosanoate, glyceryl trimyristate, glyceryl tridecenoate, glycerol tristearate and mixtures thereof; sucrose fatty acid esters such as sucrose distearate and sucrose palmitate; sorbitan fatty acid esters such as sorbitan monostearate, sorbitan monopalmitate and sorbitan tristearate; C₁₆-C₁₈ fatty alcohols such as cetyl alcohol, myristyl alcohol, stearyl alcohol, and cetostearyl alcohol; esters of fatty alcohols and fatty acids such as cetyl palmitate and cetearyl palmitate; anhydrides of fatty acids such as stearic anhydride; phospholipids including phosphatidylcholine (lecithin), phosphatidylserine, phosphatidylethanolamine, phosphatidylinositol, and lysoderivatives thereof; sphingosine and derivatives thereof; spingomyelins such as stearyl, palmitoyl, and tricosanyl spingomyelins; ceramides such as stearyl and palmitoyl ceramides; glycosphingolipids; lanolin and lanolin alcohols, calcium phosphate, sintered and unscintered hydoxyapatite, zeolites; and combinations and mixtures thereof.

Representative examples of patents relating to non-polymeric delivery systems and the preparation include U.S. Pat. Nos. 5,736,152; 5,888,533; 6,120,789; 5,968,542; and 5,747,058.

Within certain embodiments of the invention, the therapeutic compositions are provided that include (i) a fibrosis-inhibiting agent and/or (ii) an anti-infective agent. The therapeutic compositions may include one or more additional therapeutic agents (such as described above), for example, anti-inflammatory agents, anti-thrombotic agents, and/ or anti-platelet agents. Other agents that may be combined with the therapeutic compositions include, e.g., additional ingredients such as surfactants (e.g., PLURONICS, such as F-127, L-122, L-101, L-92, L-81, and L-61), preservatives, anti-oxidants.

In one aspect, the present invention provides compositions comprising i) an anti-fibrotic agent and ii) a polymer or a compound that forms a polymer in situ. The following are some, but by no means all, of the preferred anti-fibrotic agents and classes of anti-fibrotic agents that may be included in the inventive compositions:
1a. An anti-fibrotic agent that inhibits cell regeneration.
2a. An anti-fibrotic agent that inhibits angiogenesis.
3a. An anti-fibrotic agent that inhibits fibroblast migration.
4a. An anti-fibrotic agent that inhibits fibroblast proliferation.
5a. An anti-fibrotic agent that inhibits deposition of extracellular matrix.
6a. An anti-fibrotic agent inhibits tissue remodeling.
7a. An anti-fibrotic agent that is an angiogenesis inhibitor.
8a. An anti-fibrotic agent that is a 5-lipoxygenase inhibitor or antagonist.
9a. An anti-fibrotic agent that is a chemokine receptor antagonist.
10a. An anti-fibrotic agent that is a cell cycle inhibitor.
11a. An anti-fibrotic agent that is a taxane.
12a. An anti-fibrotic agent that is an anti-microtubule agent.
13a. An anti-fibrotic agent that is paclitaxel.
14a. An anti-fibrotic agent that is a cathepsin inhibitor.
15a. An anti-fibrotic agent that is an analogue or derivative of paclitaxel.
16a. An anti-fibrotic agent that is a vinca alkaloid.
17a. An anti-fibrotic agent that is camptothecin or an analogue or derivative thereof.
18a. An anti-fibrotic agent that is a podophyllotoxin.
19a. An anti-fibrotic agent that is etoposide or an analogue or derivative thereof.
20a. An anti-fibrotic agent that is an anthracycline.
21 a. An anti-fibrotic agent that is doxorubicin or an analogue or derivative thereof.
22a. An anti-fibrotic agent that mitoxantrone or an analogue or derivative thereof.
23a. An anti-fibrotic agent that is a platinum compound.
24a. An anti-fibrotic agent that is a nitrosourea.
25a. An anti-fibrotic agent that is a nitroimidazole.
26a. An anti-fibrotic agent that is a folic acid antagonist.
27a. An anti-fibrotic agent that is a cytidine analogue.
28a. An anti-fibrotic agent that is a pyrimidine analogue.
29a. An anti-fibrotic agent that is a fluoropyrimidine analogue.
30a. An anti-fibrotic agent that is a purine analogue.
31a. An anti-fibrotic agent that is a nitrogen mustard or an analogue or derivative thereof.
32a. An anti-fibrotic agent that is a hydroxyurea.
33a. An anti-fibrotic agent that is a mytomicin or an analogue or derivative thereof.
34a. An anti-fibrotic agent that is an alkyl sulfonate.
35a. An anti-fibrotic agent that is a benzamide or an analogue or derivative thereof.
36a. An anti-fibrotic agent that is a nicotinamide or an analogue or derivative thereof.
37a. An anti-fibrotic agent that is a halogenated sugar or an analogue or derivative thereof.
38a. An anti-fibrotic agent that is a DNA alkylating agent.
39a. An anti-fibrotic agent that is an anti-microtubule agent.
40a. An anti-fibrotic agent that is a topoisomerase inhibitor.
41a. An anti-fibrotic agent that is a DNA cleaving agent.
42a. An anti-fibrotic agent that is an antimetabolite.
43a. An anti-fibrotic agent inhibits adenosine deaminase.
44a. An anti-fibrotic agent inhibits purine ring synthesis.
45a. An anti-fibrotic agent that is a nucleotide interconversion inhibitor.
46a. An anti-fibrotic agent inhibits dihydrofolate reduction.
47a. An anti-fibrotic agent blocks thymidine monophosphate.
48a. An anti-fibrotic agent causes DNA damage.
49a. An anti-fibrotic agent that is a DNA intercalation agent.
50a. An anti-fibrotic agent that is a RNA synthesis inhibitor.
51a. An anti-fibrotic agent that is a pyrimidine synthesis inhibitor.
52a. An anti-fibrotic agent that inhibits ribonucleotide synthesis or function.
53a. An anti-fibrotic agent that inhibits thymidine monophosphate synthesis or function.
54a. An anti-fibrotic agent that inhibits DNA synthesis.
55a. An anti-fibrotic agent that causes DNA adduct formation.
56a. An anti-fibrotic agent that inhibits protein synthesis.
57a. An anti-fibrotic agent that inhibits microtubule function.
58a. An anti-fibrotic agent that is a cyclin dependent protein kinase inhibitor.
59a. An anti-fibrotic agent that is an epidermal growth factor kinase inhibitor.
60a. An anti-fibrotic agent that is an elastase inhibitor.
61 a. An anti-fibrotic agent that is a factor Xa inhibitor.
62a. An anti-fibrotic agent that is a farnesyltransferase inhibitor.
63a. An anti-fibrotic agent that is a fibrinogen antagonist.
64a. An anti-fibrotic agent that is a guanylate cyclase stimulant.
65a. An anti-fibrotic agent that is a heat shock protein 90 antagonist.
66a. An anti-fibrotic agent that is geldanamycin or an analogue or derivative thereof.
67a. An anti-fibrotic agent that is a guanylate cyclase stimulant.
68a. An anti-fibrotic agent that is a HMGCOA reductase inhibitor.
69a. An anti-fibrotic agent that is simvastatin or an analogue or derivative thereof.
70a. An anti-fibrotic agent that is a hydroorotate dehydrogenase inhibitor.
71a. An anti-fibrotic agent that is an IKK2 inhibitor.
72a. An anti-fibrotic agent that is an IL-1 antagonist.
73a. An anti-fibrotic agent that is an ICE antagonist.
74a. An anti-fibrotic agent that is an IRAK antagonist.
75a. An anti-fibrotic agent that is an IL-4 agonist.
76a. An anti-fibrotic agent that is an immunomodulatory agent.
77a. An anti-fibrotic agent that is sirolimus or an analogue or derivative thereof.
78a. An anti-fibrotic agent that is a nitric oxide inhibitor.
79a. An anti-fibrotic agent that is everolimus or an analogue or derivative thereof.
80a. An anti-fibrotic agent that is tacrolimus or an analogue or derivative thereof.
81a. An anti-fibrotic agent that is a TNF alpha inhibitor.
82a. An anti-fibrotic agent that is biolmus or an analogue or derivative thereof.
83a. An anti-fibrotic agent that is tresperimus or an analogue or derivative thereof.
84a. An anti-fibrotic agent that is auranofin or an analogue or derivative thereof.
85a. An anti-fibrotic agent that is 27-0-demethylrapamycin or an analogue or derivative thereof.
86a. An anti-fibrotic agent that is gusperimus or an analogue or derivative thereof.
87a. An anti-fibrotic agent that is pimecrolimus or an analogue or derivative thereof.
88a. An anti-fibrotic agent that is ABT-578 or an analogue or derivative thereof.
89a. An anti-fibrotic agent that is an inosine monophosphate dehydrogenase (IMPDH) inhibitor.
90a. An anti-fibrotic agent that is mycophenolic acid or an analogue or derivative thereof.
91a. An anti-fibrotic agent that is 1-alpha-25 dihydroxy vitamin D₃ or an analogue or derivative thereof.
92a. An anti-fibrotic agent that is a leukotriene inhibitor.
93a. An anti-fibrotic agent that is a MCP-1 antagonist.
94a. An anti-fibrotic agent that is a MMP inhibitor.
95a. An anti-fibrotic agent that is an NF kappa B inhibitor.
96a. An anti-fibrotic agent that is an NF kappa B inhibitor, wherein the NF kappa B inhibitor is Bay 11-7082.
97a. An anti-fibrotic agent that is an NO antagonist.
98a. An anti-fibrotic agent that is a p38 MAP kinase inhibitor.
99a. An anti-fibrotic agent that is a p38 MAP kinase inhibitor, wherein the p38 MAP kinase inhibitor is SB 202190.
100a. An anti-fibrotic agent that is a phosphodiesterase inhibitor.
101a. An anti-fibrotic agent that is a TGF beta inhibitor.
102a. An anti-fibrotic agent that is a thromboxane A2 antagonist.
103a. An anti-fibrotic agent that is a TNF alpha antagonist.
104a. An anti-fibrotic agent that is a TACE inhibitor.
105a. An anti-fibrotic agent that is a tyrosine kinase inhibitor.
106a. An anti-fibrotic agent that is a vitronectin inhibitor.
107a. An anti-fibrotic agent that is a fibroblast growth factor inhibitor.
108a. An anti-fibrotic agent that is a protein kinase inhibitor.
109a. An anti-fibrotic agent that is a PDGF receptor kinase inhibitor.
110a. An anti-fibrotic agent that is an endothelial growth factor receptor kinase inhibitor.
111a. An anti-fibrotic agent that is a retinoic acid receptor antagonist.
112a. An anti-fibrotic agent that is a platelet derived growth factor receptor kinase inhibitor.
113a. An anti-fibrotic agent that is a fibrinogen antagonist.
114a. An anti-fibrotic agent that is an antimycotic agent.
115a. An anti-fibrotic agent that is an antimycotic agent, wherein the antimycotic agent that is sulconizole.
116a. An anti-fibrotic agent that is a bisphosphonate.
117a. An anti-fibrotic agent that is a phospholipase Al inhibitor.
118a. An anti-fibrotic agent that is a histamine H1/H2/H3 receptor antagonist.
119a. An anti-fibrotic agent that is a macrolide antibiotic.
120a. An anti-fibrotic agent that is a GPIIb/IIIa receptor antagonist.
121a. An anti-fibrotic agent that is an endothelin receptor antagonist.
122a. An anti-fibrotic agent that is a peroxisome proliferator-activated receptor agonist.
123a. An anti-fibrotic agent that is an estrogen receptor agent.
124a. An anti-fibrotic agent that is a somastostatin analogue.
125a. An anti-fibrotic agent that is a neurokinin 1 antagonist.
126a. An anti-fibrotic agent that is a neurokinin 3 antagonist.
127a. An anti-fibrotic agent that is a VLA-4 antagonist.
128a. An anti-fibrotic agent that is an osteoclast inhibitor.
129a. An anti-fibrotic agent that is a DNA topoisomerase ATP hydrolyzing inhibitor.
130a. An anti-fibrotic agent that is an angiotensin I converting enzyme inhibitor.
131a. An anti-fibrotic agent that is an angiotensin II antagonist.
132a. An anti-fibrotic agent that is an enkephalinase inhibitor.
133a. An anti-fibrotic agent that is a peroxisome proliferator-activated receptor gamma agonist insulin sensitizer.
134a. An anti-fibrotic agent that is a protein kinase C inhibitor.
135a. An anti-fibrotic agent that is a ROCK (rho-associated kinase) inhibitor.
136a. An anti-fibrotic agent that is a CXCR3 inhibitor.
137a. An anti-fibrotic agent that is an Itk inhibitor.
138a. An anti-fibrotic agent that is a cytosolic phospholipase A₂-alpha inhibitor.
139a. An anti-fibrotic agent that is a PPAR agonist.
140a. An anti-fibrotic agent that is an immunosuppressant.
141a. An anti-fibrotic agent that is an Erb inhibitor.
142a. An anti-fibrotic agent that is an apoptosis agonist.
143a. An anti-fibrotic agent that is a lipocortin agonist.
144a. An anti-fibrotic agent that is a VCAM-1 antagonist.
145a. An anti-fibrotic agent that is a collagen antagonist.

As mentioned above, the present invention provides compositions comprising each of the foregoing 146 (i.e., 1a through 145a) listed anti-fibrotic agents or classes of anti-fibrotic agents, with each of the following 98 (i.e., 1b through 97b) polymers and compounds:
1b. A crosslinked polymer.
2b. A polymer that reacts with mammalian tissue.
3b. A polymer that is a naturally occurring polymer.
4b. A polymer that is a protein.
5b. A polymer that is a carbohydrate.
6b. A polymer that is biodegradable.
7b. A polymer that is crosslinked and biodegradable.
8b. A polymer that nonbiodegradable.
9b. Collagen.
10b. Methylated collagen.
11b. Fibrinogen.
12b. Thrombin.
13b. Albumin.
14b. Plasminogen.
15b. von Willebrands factor.
16b. Factor VIII.
17b. Hypoallergenic collagen.
18b. Atelopeptidic collagen.
19b. Telopeptide collagen.
20b. Crosslinked collagen.
21b. Aprotinin.
22b. Gelatin.
23b. A protein conjugate.
24b. A gelatin conjugate.
25b. Hyaluronic acid.
26b. A hyaluronic acid derivative.
27b. A synthetic polymer.
28b. A polymer formed from reactants comprising a synthetic isocyanate-containing compound.
29b. A synthetic isocyanate-containing compound.
30b. A polymer formed from reactants comprising a synthetic thiol-containing compound.
31b. A synthetic thiol-containing compound.
32b. A polymer formed from reactants comprising a synthetic compound containing at least two thiol groups.
33b. A synthetic compound containing at least two thiol groups.
34b. A polymer formed from reactants comprising a synthetic compound containing at least three thiol groups.
35b. A synthetic compound containing at least three thiol groups.
36b. A polymer formed from reactants comprising a synthetic compound containing at least four thiol groups.
37b. A synthetic compound containing at least four thiol groups.
38b. A polymer formed from reactants comprising a synthetic amino-containing compound.
39b. A synthetic amino-containing compound.
40b. A polymer formed from reactants comprising a synthetic compound containing at least two amino groups.
41b. A synthetic compound containing at least two amino groups.
42b. A polymer formed from reactants comprising a synthetic compound containing at least three amino groups.
43b. A synthetic compound containing at least three amino groups.
44b. A polymer formed from reactants comprising a synthetic compound containing at least four amino groups.
45b. A synthetic compound containing at least four amino groups.
46b. A polymer formed from reactants comprising a synthetic compound comprising a carbonyl-oxygen-succinimidyl group.
47b. A synthetic compound comprising a carbonyl-oxygen-succinimidyl group.
48b. A polymer formed from reactants comprising a synthetic compound comprising at least two carbonyl-oxygen-succinimidyl groups.
49b. A synthetic compound comprising at least two carbonyl-oxygen-succinimidyl groups.
50b. A polymer formed from reactants comprising a synthetic compound comprising at least three carbonyl-oxygen-succinimidyl groups.
51b. A synthetic compound comprising at least three carbonyl-oxygen-succinimidyl groups.
52b. A polymer formed from reactants comprising a synthetic compound comprising at least four carbonyl-oxygen-succinimidyl groups.
53b. A synthetic compound comprising at least four carbonyl-oxygen-succinimidyl groups.
54b. A polymer formed from from reactants comprising a synthetic polyalkylene oxide-containing compound.
55b. A synthetic polyalkylene oxide-containing compound.
56b. A polymer formed from reactants comprising a synthetic compound comprising both polyalkylene oxide and biodegradable polyester blocks.
57b. A synthetic compound comprising both polyalkylene oxide and biodegradable polyester blocks.
58b. A polymer formed from reactants comprising a synthetic polyalkylene oxide-containing compound having reactive amino groups.
59b. A synthetic polyalkylene oxide-containing compound having reactive amino groups.
60b. A polymer formed from reactants comprising a synthetic polyalkylene oxide-containing compound having reactive thiol groups.
61b. A synthetic polyalkylene oxide-containing compound having reactive thiol groups.
62b. A polymer formed from reactants comprising a synthetic polyalkylene oxide-containing compound having reactive carbonyl-oxygen-succinimidyl groups.
63b. A synthetic polyalkylene oxide-containing compound having reactive carbonyl-oxygen-succinimidyl groups.
64b. A polymer formed from reactants comprising a synthetic compound comprising a biodegradable polyester block.
65b. A synthetic compound comprising a biodegradable polyester block.
66b. A polymer formed from reactants comprising a synthetic polymer formed in whole or part from lactic acid or lactide.
67b. A synthetic polymer formed in whole or part from lactic acid or lactide.
68b. A polymer formed from reactants comprising a synthetic polymer formed in whole or part from glycolic acid or glycolide.
69b. A synthetic polymer formed in whole or part from glycolic acid or glycolide.
70b. A polymer formed from reactants comprising polylysine.
71b. Polylysine.
72b. A polymer formed from reactants comprising (a) protein and (b) a compound comprising a polyalkylene oxide portion.
73b. A polymer formed from reactants comprising (a) protein and (b) polylysine.
74b. A polymer formed from reactants comprising (a) protein and (b) a compound having at least four thiol groups.
75b. A polymer formed from reactants comprising (a) protein and (b) a compound having at least four amino groups.
76b. A polymer formed from reactants comprising (a) protein and (b) a compound having at least four carbonyl-oxygen-succinimide groups.
77b. A polymer formed from reactants comprising (a) protein and (b) a compound having a biodegradable region formed from reactants selected from lactic acid, lactide, glycolic acid, glycolide, and epsilon-caprolactone.
78b. A polymer formed from reactants comprising (a) collagen and (b) a compound comprising a polyalkylene oxide portion.
79b. A polymer formed from reactants comprising (a) collagen and (b) polylysine.
80b. A polymer formed from reactants comprising (a) collagen and (b) a compound having at least four thiol groups.
81b. A polymer formed from reactants comprising (a) collagen and (b) a compound having at least four amino groups.
82b. A polymer formed from reactants comprising (a) collagen and (b) a compound having at least four carbonyl-oxygen-succinimide groups.
83b. A polymer formed from reactants comprising (a) collagen and (b) a compound having a biodegradable region formed from reactants selected from lactic acid, lactide, glycolic acid, glycolide, and epsilon-caprolactone.
84b. A polymer formed from reactants comprising (a) methylated collagen and (b) a compound comprising a polyalkylene oxide portion.
85b. A polymer formed from reactants comprising (a) methylated collagen and (b) polylysine.
86b. A polymer formed from reactants comprising (a) methylated collagen and (b) a compound having at least four thiol groups.
87b. A polymer formed from reactants comprising (a) methylated collagen and (b) a compound having at least four amino groups.
88b. A polymer formed from reactants comprising (a) methylated collagen and (b) a compound having at least four carbonyl-oxygen-succinimide groups.
89b. A polymer formed from reactants comprising (a) methylated collagen and (b) a compound having a biodegradable region formed from reactants selected from lactic acid, lactide, glycolic acid, glycolide, and epsilon-caprolactone.
90b. A polymer formed from reactants comprising hyaluronic acid.
91b. A polymer formed from reactants comprising a hyaluronic acid derivative.
92b. A polymer formed from reactants comprising pentaerythritol poly(ethylene glycol)ether tetra-sulfhydryl of number average molecular weight between 3,000 and 30,000.
93b. Pentaerythritol poly(ethylene glycol)ether tetra-sulfhydryl of number average molecular weight between 3,000 and 30,000.
94b. A polymer formed from reactants comprising pentaerythritol poly(ethylene glycol)ether tetra-amino of number average molecular weight between 3,000 and 30,000.
95b. Pentaerythritol poly(ethylene glycol)ether tetra-amino of number average molecular weight between 3,000 and 30,000.
96b. A polymer formed from reactants comprising (a) a synthetic compound having a number average molecular weight between 3,000 and 30,000 and comprising a polyalkylene oxide region and multiple nucleophilic groups, and (b) a synthetic compound having a number average molecular weight between 3,000 and 30,000 and comprising a polyalkylene oxide region and multiple electrophilic groups.
97b. A mixture of (a) a synthetic compound having a number average molecular weight between 3,000 and 30,000 and comprising a polyalkylene oxide region and multiple nucleophilic groups, and (b) a synthetic compound having a number average molecular weight between 3,000 and 30,000 and comprising a polyalkylene oxide region and multiple electrophilic groups.

As mentioned above, the present invention provides compositions comprising each of the foregoing 146 (1a through 145a) listed anti-fibrotic agents or classes of anti-fibrotic agents, with each of the foregoing 98 (1b through 97b) polymers and compounds: Thus, in separate aspects, the invention provides 146 times 98=14,308 described compositions. In other words, each of the following is a distinct aspect of the present invention: 1a+1b; 1a+2b; 1a+3b; 1a+4b; 1a+5b; 1a+6b; 1a+7b; 1a+8b; 1a+9b; 1a+10b; 1a+11b; 1a+12b; 1a+13b; 1a+14b; 1a+15b; 1a+16b; 1a+17b; 1a+18b; 1a+19b; 1a+20b; 1a+21b; 1a+22b; 1a+23b; 1a+24b; 1a+25b; 1a+26b; 1a+27b; 1a+28b; 1a+29b; 1a+30b; 1a+31b; 1a+32b; 1a+33b; 1a+34b; 1a+35b; 1a+36b; 1a+37b; 1a+38b; 1a+39b; 1a+40b; 1a+41b; 1a+42b; 1a+43b; 1a+44b; 1a+45b; 1a+46b; 1a+47b; 1a+48b; 1a+49b; 1a+50b; 1a+51b; 1a+52b; 1a+53b; 1a+54b; 1a+55b; 1a+55b; 1a+57b; 1a+58b; 1a+59b; 1a+60b; 1a+61b; 1a+62b; 1a+63b; 1a+64b; 1a+65b; 1a+66b; 1a+67b; 1a+68b; 1a+69b; 1a+70b; 1a+71b; 1a+72b; 1a+73b; 1a+74b; 1a+75b; 1a+76b; 1a+77b; 1a+78b; 1a+79b; 1a+80b; 1a+81b; 1a+82b; 1a+83b; 1a+84b; 1a+85b; 1a+86b; 1a+87b; 1a+88b; 1a+89b; 1a+90b; 1a+91b; 1a+92b; 1a+93b; 1a+94b; 1a+95b; 1a+96b; 1a+97b; 2a+1b; 2a +2b; 2a +3b; 2a+4b; 2a+5b; 2a+6b; 2a+7b; 2a+8b; 2a+9b; 2a+10b; 2a+11b; 2a+12b; 2a+13b; 2a+14b; 2a+15b; 2a+16b; 2a+17b; 2a+18b; 2a+19b; 2a+20b; 2a+21b; 2a+22b; 2a+23b; 2a+24b; 2a+25b; 2a+26b; 2a+27b; 2a+28b; 2a+29b; 2a+30b; 2a+31b; 2a+32b; 2a+33b; 2a+34b; 2a+35b; 2a+36b; 2a+37b; 2a+38b; 2a+39b; 2a+40b; 2a+41b; 2a+42b; 2a+43b; 2a+44b; 2a+45b; 2a+46b; 2a+47b; 2a+48b; 2a+49b; 2a+50b; 2a+51b; 2a+52b; 2a+53b; 2a+54b; 2a+55b; 2a+55b; 2a+57b; 2a+58b; 2a+59b; 2a+60b; 2a+61b; 2a+62b; 2a+63b; 2a+64b; 2a+65b; 2a+66b; 2a+67b; 2a+68b; 2a+69b; 2a+70b; 2a+71b; 2a+72b; 2a+73b; 2a+74b; 2a+75b; 2a+76b; 2a+77b; 2a+78b; 2a+79b; 2a+80b; 2a+81b; 2a+82b; 2a+83b; 2a+84b; 2a+85b; 2a+86b; 2a+87b; 2a+88b; 2a+89b; 2a+90b; 2a+91b; 2a+92b; 2a+93b; 2a+94b; 2a+95b; 2a+96b; 2a+97b; 3a+22b; 3a+23b; 3a+24b; 3a+25b; 3a+26b; 3a+27b; 3a+28b; 3a+29b; 3a+30b; 3a+31b; 3a+32b; 3a+33b; 3a+34b; 3a+35b; 3a+36b; 3a+37b; 3a+38b; 3a+39b; 3a+40b; 3a+41b; 3a+42b; 3a+43b; 3a+44b; 3a+45b; 3a+46b; 3a+47b; 3a+48b; 3a+49b; 3a+50b; 3a+51b; 3a+52b; 3a+53b; 3a+54b; 3a+55b; 3a+55b; 3a+56b; 3a+57b; 3a+58b; 3a+60b; 3a+61b; 3a+62b; 3a+63b; 3a+64b; 3a+65b; 3a+66b; 3a+67b; 3a+68b; 3a+69b; 3a+70b; 3a+71b; 3a+72b; 3a+73b; 3a+74b; 3a+75b; 3a+76b; 3a+77b; 3a+78b; 3a+79b; 3a+80b; 3a+81b; 3a+82b; 3a+83b; 3a+84b; 3a+85b; 3a+86b; 3a+87b; 3a+88b; 3a+89b; 3a+90b; 3a+91b; 3a+92b; 3a+93b; 3a+94b; 3a+95b; 3a+96b; 3a+97b; 4a+12b; 4a+13b; 4a+14b; 4a+15b; 4a+16b; 4a+17b; 4a+18b; 4a+19b; 4a+20b; 4a+21b; 4a+22b; 4a+23b; 4a+24b; 4a+25b; 4a+26b; 4a+27b; 4a+28b; 4a+29b; 4a+30b; 4a+31b; 4a+32b; 4a+33b; 4a+34b; 4a+35b; 4a+36b; 4a+37b; 4a+38b; 4a+39b; 4a+40b; 4a+41b; 4a+42b; 4a+43b; 4a+44b; 4a+45b; 4a+46b; 4a+47b; 4a+48b; 4a+49b; 4a+50b; 4a+51b; 4a+52b; 4a+53b; 4a+54b; 4a+55b; 4a+55b; 4a+57b; 4a+58b; 4a+59b; 4a+60b; 4a+61b; 4a+62b; 4a+63b; 4a+64b; 4a+65b; 4a+66b; 4a+67b; 4a+68b; 4a+69b; 4a+70b; 4a+71b; 4a+72b; 4a+73b; 4a+74b; 4a+75b; 4a+76b; 4a+77b; 4a+78b; 4a+79b; 4a+80b; 4a+81b; 4a+82b; 4a+83b; 4a+84b; 4a+85b; 4a+86b; 4a+87b; 4a+88b; 4a+89b; 4a+90b; 4a+91b; 4a+92b; 4a+93b; 4a+94b; 4a+95b; 4a+96b; 4a+97b; 5a+12b; 5a+13b; 5a+14b; 5a+15b; 5a+16b; 5a+17b; 5a+18b; 5a+19b; 5a+20b; 5a+21b; 5a+22b; 5a+23b; 5a+24b; 5a+25b; 5a+26b; 5a+27b; 5a+28b; 5a+29b; 5a+30b; 5a+31b; 5a+32b; 5a+33b; 5a+34b; 5a+35b; 5a+36b; 5a+37b; 5a+38b; 5a+39b; 5a+40b; 5a+41b; 5a+42b; 5a+43b; 5a+44b; 5a+45b; 5a+46b; 5a+47b; 5a+48b; 5a+49b; 5a+50b; 5a+51b; 5a+52b; 5a+53b; 5a+54b; 5a+55b; 5a+56b; 5a+57b; 5a+58b; 5a+59b; 5a+60b; 5a+61b; 5a+62b; 5a+63b; 5a+64b; 5a+65b; 5a+66b; 5a+67b; 5a+68b; 5a+69b; 5a+70b; 5a+71b; 5a+72b; 5a+73b; 5a+74b; 5a+75b; 5a+76b; 5a+77b; 5a+78b; 5a+79b; 5a+80b; 5a+81b; 5a+82b; 5a+83b; 5a+84b; 5a+85b; 5a+86b; 5a+87b; 5a+88b; 5a+89b; 5a+90b; 5a+91b; 5a+92b; 5a+93b; 5a+94b; 5a+95b; 5a+96b; 5a+97b; 6a+1b; 6a+2b; 6a+3b; 6a+4b; 6a+5b; 6a+6b; 6a+7b; 6a+8b; 6a+9b; 6a+10b; 6a+11b; 6a+12b; 6a+13b; 6a+14b; 6a+15b; 6a+16b; 6a+17b; 6a+18b; 6a+19b; 6a+20b; 6a+21b; 6a+22b; 6a+23b; 6a+24b; 6a+25b; 6a+26b; 6a+27b; 6a+28b; 6a+29b; 6a+30b; 6a+31b; 6a+32b; 6a+33b; 6a+34b; 6a+35b; 6a+36b; 6a+37b; 6a+38b; 6a+39b; 6a+40b; 6a+41b; 6a+42b; 6a+43b; 6a+44b; 6a+45b; 6a+46b; 6a+47b; 6a+48b; 6a+49b; 6a+50b; 6a+51b; 6a+51b; 6a+53b; 6a+54b; 6a+55b; 6a+55b; 6a+57b; 6a+58b; 6a+59b; 6a+60b; 6a+61b; 6a+62b; 6a+63b; 6a+64b; 6a+65b; 6a+66b; 6a+67b; 6a+68b; 6a+69b; 6a+70b; 6a+71b; 6a+72b; 6a+73b; 6a+74b; 6a+75b; 6a+76b; 6a+77b; 6a+78b; 6a+79b; 6a+80b; 6a+81b; 6a+82b; 6a+83b; 6a+84b; 6a+85b; 6a+86b; 6a+87b; 6a+88b; 6a+89b; 6a+90b; 6a+91b; 6a+92b; 6a+93b; 6a+94b; 6a+95b; 6a+96b; 6a+97b; 7a+1b; 7a+2b; 7a+3b; 7a+4b; 7a+5b; 7a+6b; 7a+7b; 7a+8b; 7a+9b; 7a+10b; 7a+11b; 7a+12b; 7a+13b; 7a+14b; 7a+15b; 7a+16b; 7a+17b; 7a+18b; 7a+19b; 7a+20b; 7a+21b; 7a+22b; 7a+23b; 7a+24b; 7a+25b; 7a+26b; 7a+27b; 7a+28b; 7a+29b; 7a+30b; 7a+31b; 7a+32b; 7a+33b; 7a+34b; 7a+35b; 7a+36b; 7a+37b; 7a+38b; 7a+39b; 7a+40b; 7a+41b; 7a+42b; 7a+43b; 7a+44b; 7a+45b; 7a+46b; 7a+47b; 7a+48b; 7a+49b; 7a+50b; 7a+51b; 7a+52b; 7a+53b; 7a+54b; 7a+55b; 7a+55b; 7a+57b; 7a+58b; 7a+59b; 7a+60b; 7a+61b; 7a+62b; 7a+62b; 7a+64b; 7a+65b; 7a+66b; 7a+67b; 7a+68b; 7a+69b; 7a+70b; 7a+71b; 7a+72b; 7a+73b; 7a+74b; 7a+75b; 7a+76b; 7a+77b; 7a+78b; 7a+79b; 7a+80b; 7a+81b; 7a+82b; 7a+83b; 7a+84b; 7a+85b; 7a+86b; 7a+87b; 7a+88b; 7a+89b; 7a+90b; 7a+91b; 7a+92b; 7a+93b; 7a+94b; 7a+95b; 7a+96b; 7a+97b; 8a+12b; 8a+13b; 8a+14b; 8a+15b; 8a+16b; 8a+17b; 8a+18b; 8a+19b; 8a+20b; 8a+21b; 8a+22b; 8a+23b; 8a+24b; 8a+25b; 8a+26b; 8a+27b; 8a+28b; 8a+29b; 8a+30b; 8a+31b; 8a+32b; 8a+33b; 8a+34b; 8a+35b; 8a+36b; 8a+37b; 8a+38b; 8a+39b; 8a+40b; 8a+41b; 8a+42b; 8a+43b; 8a+44b; 8a+45b; 8a+46b; 8a+47b; 8a+48b; 8a+49b; 8a+50b; 8a+51b; 8a+52b; 8a+53b; 8a+54b; 8a+55b; 8a+55b; 8a+57b; 8a+58b; 8a+59b; 8a+60b; 8a+61b; 8a+62b; 8a+63b; 8a+64b; 8a+65b; 8a+66b; 8a+67b; 8a+68b; 8a+69b; 8a+70b; 8a+71b; 8a+72b; 8a+73b; 8a+74b; 8a+75b; 8a+76b; 8a+77b; 8a+78b; 8a+79b; 8a+80b; 8a+81b; 8a+82b; 8a+83b; 8a+84b; 8a+85b; 8a+86b; 8a+87b; 8a+88b; 8a+89b; 8a+90b; 8a+91b; 8a+92b; 8a+93b; 8a+94b; 8a+95b; 8a+96b; 8a+97b; 9a+1b; 9a+2b; 9a+3b; 9a+4b; 9a+5b; 9a+6b; 9a+7b; 9a+8b; 9a+9b; 9a+10b; 9a+11b; 9a+12b; 9a+13b; 9a+14b; 9a+15b; 9a+16b; 9a+17b; 9a+18b; 9a+19b; 9a+20b; 9a+21b; 9a+22b; 9a+23b; 9a+24b; 9a+25b; 9a+26b; 9a+27b; 9a+28b; 9a+29b; 9a+30b; 9a+31b; 9a+32b; 9a+33b; 9a+34b; 9a+35b; 9a+36b; 9a+37b; 9a+38b; 9a+39b; 9a+40b; 9a+41b; 9a+42b; 9a+43b; 9a+44b; 9a+45b; 9a+46b; 9a+47b; 9a+48b; 9a+49b; 9a+50b; 9a+51b; 9a+52b; 9a+53b; 9a+54b; 9a+55b; 9a+55b; 9a+57b; 9a+58b; 9a+59b; 9a+60b; 9a+61b; 9a+62b; 9a+63b; 9a+64b; 9a+65b; 9a+66b; 9a+67b; 9a+68b; 9a+69b; 9a+70b; 9a+71b; 9a+72b; 9a+73b; 9a+74b; 9a+75b; 9a+76b; 9a+77b; 9a+78b; 9a+79b; 9a+80b; 9a+81b; 9a+82b; 9a+83b; 9a+84b; 9a+85b; 9a+86b; 9a+87b; 9a+88b; 9a+89b; 9a+90b; 9a+91b; 9a+92b; 9a+93b; 9a+94b; 9a+95b; 9a+96b; 9a+97b; 10a+1b; 10a+2b; 10a+3b; 10a+4b; 10a+5b; 10a+6b; 10a+7b; 10a+8b; 10a+9b; 10a+10b; 10a+11b; 10a+12b; 10a+13b; 10a+14b; 10a+15b; 10a+16b; 10a+17b; 10a+18b; 10a+19b; 10a+20b; 10a+21b; 10a+22b; 10a+23b; 10a+24b; 10a+25b; 10a+26b; 10a+27b; 10a+28b; 10a+29b; 10a+30b; 10a+31b; 10a+32b; 10a+33b; 10a+34b; 10a+35b; 10a+36b; 10a+37b; 10a+38b; 10a+39b; 10a+40b; 10a+41b; 10a+42b; 10a+43b; 10a+44b; 10a+45b; 10a+46b; 10a+47b; 10a+48b; 10a+49b; 10a+50b; 10a+51b; 10a+52b; 10a+53b; 10a+54b; 10a+55b; 10a+55b; 10a+57b; 10a+58b; 10a+59b; 10a+60b; 10a+61b; 10a+62b; 10a+63b; 10a+64b; 10a+65b; 10a+66b; 10a+67b; 10a+68b; 10a+69b; 10a+70b; 10a+71b; 10a+72b; 10a+73b; 10a+74b; 10a+75b; 10a+76b; 10a+77b; 10a+78b; 10a+79b; 10a+80b; 10a+81b; 10a+82b; 10a+83b; 10a+84b; 10a+85b; 10a+86b; 10a+87b; 10a+88b; 10a+89b; 10a+90b; 10a+91b; 10a+92b; 10a+93b; 10a+94b; 10a+95b; 10a+96b; 10a+97b; 11a+1b; 11a+2b; 11a+3b; 11a+4b; 11a+5b; 11a+6b; 11a+7b; 11a+8b; 11a+9b; 11a+10b; 11a+11b; 11a+12b; 11a+13b; 11a+14b; 11a+15b; 11a+16b; 11a+17b; 11a+18b; 11a+19b; 11a+20b; 11a+21b; 11a+22b; 11a+23b; 11a+24b; 11a+25b; 11a+26b; 11a+27b; 11a+28b; 11a+29b; 11a+30b; 11a+31b; 11a+32b; 11a+33b; 11a+34b; 11a+35b; 11a+36b; 11a+37b; 11a+38b; 11a+39b; 11a+40b; 11a+41b; 11a+42b; 11a+43b; 11a+44b; 11a+45b; 11a+46b; 11a+47b; 11a+48b; 11a+49b; 11a+50b; 11a+51b; 11a+52b; 11a+53b; 11a+54b; 11a+55b; 11a+55b; 11a+57b; 11a+58b; 11a+59b; 11a+60b; 11a+61b; 11a+62b; 11a+63b; 11a+64b; 11a+65b; 11a+66b; 11a+67b; 11a+68b; 11a+69b; 11a+70b; 11a+71b; 11a+72b; 11a+73b; 11a+74b; 11a+75b; 11a+76b; 11a+77b; 11a+78b; 11a+79b; 11a+80b; 11a+81b; 11a+82b; 11a+83b; 11a+84b; 11a+85b; 11a+86b; 11a+87b; 11a+88b; 11a+89b; 11a+90b; 11a+91b; 11a+92b; 11a+93b; 11a+94b; 11a+95b; 11a+96b; 11a+97b; 12a+1b; 12a+2b; 12a+3b; 12a+4b; 12a+5b; 12a+6b; 12a+7b; 12a+8b; 12a+9b; 12a+10b; 12a+11b; 12a+12b; 12a+13b; 12a+14b; 12a+15b; 12a+16b; 12a+17b; 12a+18b; 12a+19b; 12a+20b; 12a+21b; 12a+22b; 12a+23b; 12a+24b; 12a+25b; 12a+26b; 12a+27b; 12a+28b; 12a+29b; 12a+30b; 12a+31b; 12a+32b; 12a+33b; 12a+34b; 12a+35b; 12a+36b; 12a+37b; 12a+38b; 12a+39b; 12a+40b; 12a+41b; 12a+42b; 12a+43b; 12a+44b; 12a+45b; 12a+46b; 12a+47b; 12a+48b; 12a+49b; 12a+50b; 12a+51b; 12a+52b; 12a+53b; 12a+54b; 12a+55b; 12a+55b; 12a+57b; 12a+58b; 12a+59b; 12a+60b; 12a+61b; 12a+62b; 12a+63b; 12a+64b; 12a+65b; 12a+66b; 12a+67b; 12a+68b; 12a+69b; 12a+70b; 12a+71b; 12a+72b; 12a+73b; 12a+74b; 12a+75b; 12a+76b; 12a+77b; 12a+78b; 12a+79b; 12a+80b; 12a+81b; 12a+82b; 12a+83b; 12a+84b; 12a+85b; 12a+86b; 12a+87b; 12a+88b; 12a+89b; 12a+90b; 12a+91b; 12a+92b; 12a+93b; 12a+94b; 12a+95b; 12a+96b; 12a+97b; 13a+1b; 13a+2b; 13a+3b; 13a+4b; 13a+5b; 13a+6b; 13a+7b; 13a+8b; 13a+9b; 13a+10b; 13a+11b; 13a+12b; 13a+13b; 13a+14b; 13a+15b; 13a+16b; 13a+17b; 13a+18b; 13a+19b; 13a+20b; 13a+21b; 13a+22b; 13a+23b; 13a+24b; 13a+25b; 13a+26b; 13a+27b; 13a+28b; 13a+29b; 13a+30b; 13a+31b; 13a+32b; 13a+33b; 13a+34b; 13a+35b; 13a+36b; 13a+37b; 13a+38b; 13a+39b; 13a+40b; 13a+41b; 13a+42b; 13a+43b; 13a+44b; 13a+45b; 13a+46b; 13a+47b; 13a+48b; 13a+49b; 13a+50b; 13a+51b; 13a+52b; 13a+53b; 13a+54b; 13a+55b; 13a+55b; 13a+57b; 13a+58b; 13a+59b; 13a+60b; 13a+61b; 13a+62b; 13a+63b; 13a+64b; 13a+65b; 13a+66b; 13a+67b; 13a+68b; 13a+69b; 13a+70b; 13a+71b; 13a+72b; 13a+73b; 13a+74b; 13a+75b; 13a+76b; 13a+77b; 13a+78b; 13a+79b; 13a+80b; 13a+81b; 13a+82b; 13a+83b; 13a+84b; 13a+85b; 13a+86b; 13a+87b; 13a+88b; 13a+89b; 13a+90b; 13a+91b; 13a+92b; 13a+93b; 13a+94b; 13a+95b; 13a+96b; 13a+97b; 14a+1b; 14a+2b; 14a+3b; 14a+4b; 14a+5b; 14a+6b; 14a+7b; 14a+8b; 14a+9b; 14a+10b; 14a+11b; 14a+12b; 14a+13b; 14a+14b; 14a+15b; 14a+16b; 14a+17b; 14a+18b; 14a+19b; 14a+20b; 14a+21b; 14a+22b; 14a+23b; 14a+24b; 14a+25b; 14a+26b; 14a+27b; 14a+28b; 14a+29b; 14a+30b; 14a+31b; 14a+32b; 14a+33b; 14a+34b; 14a+35b; 14a+36b; 14a+37b; 14a+38b; 14a+39b; 14a+40b; 14a+41b; 14a+42b; 14a+43b; 14a+44b; 14a+45b; 14a+46b; 14a+47b; 14a+48b; 14a+49b; 14a+50b; 14a+51b; 14a+52b; 14a+53b; 14a+54b; 14a+55b; 14a+55b; 14a+57b; 14a+58b; 14a+59b; 14a+60b; 14a+61b; 14a+62b; 14a+63b; 14a+64b; 14a+65b; 14a+66b; 14a+67b; 14a+68b; 14a+69b; 14a+70b; 14a+71b; 14a+72b; 14a+73b; 14a+74b; 14a+75b; 14a+76b; 14a+77b; 14a+78b; 14a+79b; 14a+80b; 14a+81b; 14a+82b; 14a+83b; 14a+84b; 14a+85b; 14a+86b; 14a+87b; 14a+88b; 14a+89b; 14a+90b; 14a+91b; 14a+92b; 14a+93b; 14a+94b; 14a+95b; 14a+96b; 14a+97b; 15a+1b; 15a+2b; 15a+3b; 15a+4b; 15a+5b; 15a+6b; 15a+7b; 15a+8b; 15a+9b; 15a+10b; 15a+11b; 15a+12b; 15a+13b; 15a+14b; 15a+15b; 15a+16b; 15a+17b; 15a+18b; 15a+19b; 15a+20b; 15a+21b; 15a+22b; 15a+23b; 15a+24b; 15a+25b; 15a+26b; 15a+27b; 15a+28b; 15a+29b; 15a+30b; 15a+31b; 15a+32b; 15a+33b; 15a+34b; 15a+35b; 15a+36b; 15a+37b; 15a+38b; 15a+39b; 15a+40b; 15a+41b; 15a+42b; 15a+43b; 15a+44b; 15a+45b; 15a+46b; 15a+47b; 15a+48b; 15a+49b; 15a+50b; 15a+51b; 15a+52b; 15a+53b; 15a+54b; 15a+55b; 15a+55b; 15a+57b; 15a+58b; 15a+59b; 15a+60b; 15a+61b; 15a+62b; 15a+63b; 15a+64b; 15a+65b; 15a+66b; 15a+67b; 15a+68b; 15a+69b; 15a+70b; 15a+71b; 15a+72b; 15a+73b; 15a+74b; 15a+75b; 15a+76b; 15a+77b; 15a+78b; 15a+79b; 15a+80b; 15a+81b; 15a+82b; 15a+83b; 15a+84b; 15a+85b; 15a+86b; 15a+87b; 15a+88b; 15a+89b; 15a+90b; 15a+91b; 15a+92b; 15a+93b; 15a+94b; 15a+95b; 15a+96b; 15a+97b; 16a+1b; 16a+2b; 16a+3b; 16a+4b; 16a+5b; 16a+6b; 16a+7b; 16a+8b; 16a+9b; 16a+10b; 16a+11b; 16a+12b; 16a+13b; 16a+14b; 16a+15b; 16a+16b; 16a+17b; 16a+18b; 16a+19b; 16a+20b; 16a+21b; 16a+22b; 16a+23b; 16a+24b; 16a+25b; 16a+26b; 16a+27b; 16a+28b; 16a+29b; 16a+30b; 16a+31b; 16a+32b; 16a+33b; 16a+34b; 16a+35b; 16a+36b; 16a+37b; 16a+38b; 16a+39b; 16a+40b; 16a+41b; 16a+42b; 16a+43b; 16a+44b; 16a+45b; 16a+46b; 16a+47b; 16a+48b; 16a+49b; 16a+50b; 16a+51b; 16a+52b; 16a+53b; 16a+54b; 16a+55b; 16a+55b; 16a+57b; 16a+58b; 16a+59b; 16a+60b; 16a+61b; 16a+62b; 16a+63b; 16a+64b; 16a+65b; 16a+66b; 16a+67b; 16a+68b; 16a+69b; 16a+70b; 16a+71b; 16a+72b; 16a+73b; 16a+74b; 16a+75b; 16a+76b; 16a+77b; 16a+78b; 16a+79b; 16a+80b; 16a+81b; 16a+82b; 16a+83b; 16a+84b; 16a+85b; 16a+86b; 16a+87b; 16a+88b; 16a+89b; 16a+90b; 16a+91b; 16a+92b; 16a+93b; 16a+94b; 16a+95b; 16a+96b; 16a+97b; 17a+1b; 17a+2b; 17a+3b; 17a+4b; 17a+5b; 17a+6b; 17a+7b; 17a+8b; 17a+9b; 17a+10b; 17a+11b; 17a+12b; 17a+13b; 17a+14b; 17a+15b; 17a+16b; 17a+17b; 17a+18b; 17a+19b; 17a+20b; 17a+21b; 17a+22b; 17a+23b; 17a+24b; 17a+25b; 17a+26b; 17a+27b; 17a+28b; 17a+29b; 17a+30b; 17a+31b; 17a+32b; 17a+33b; 17a+34b; 17a+35b; 17a+36b; 17a+37b; 17a+38b; 17a+39b; 17a+40b; 17a+41b; 17a+42b; 17a+43b; 17a+44b; 17a+45b; 17a+46b; 17a+47b; 17a+48b; 17a+49b; 17a+50b; 17a+51b; 17a+52b; 17a+53b; 17a+54b; 17a+55b; 17a+55b; 17a+57b; 17a+58b; 17a+59b; 17a+60b; 17a+61b; 17a+62b; 17a+63b; 17a+64b; 17a+65b; 17a+66b; 17a+67b; 17a+68b; 17a+69b; 17a+70b; 17a+71b; 17a+72b; 17a+73b; 17a+74b; 17a+75b; 17a+76b; 17a+77b; 17a+78b; 17a+79b; 17a+80b; 17a+81b; 17a+82b; 17a+83b; 17a+84b; 17a+85b; 17a+86b; 17a+87b; 17a+88b; 17a+89b; 17a+90b; 17a+91b; 17a+92b; 17a+93b; 17a+94b; 17a+95b; 17a+96b; 17a+97b; 18a+1b; 18a+2b; 18a+3b; 18a+4b; 18a+5b; 18a+6b; 18a+7b; 18a+8b; 18a+9b; 18a+10b; 18a+11b; 18a+12b; 18a+13b; 18a+14b; 18a+15b; 18a+16b; 18a+17b; 18a+18b; 18a+19b; 18a+20b; 18a+21b; 18a+22b; 18a+23b; 18a+24b; 18a+25b; 18a+26b; 18a+27b; 18a+28b; 18a+29b; 18a+30b; 18a+31b; 18a+32b; 18a+33b; 18a+34b; 18a+35b; 18a+36b; 18a+37b; 18a+38b; 18a+39b; 18a+40b; 18a+41b; 18a+42b; 18a+43b; 18a+44b; 18a+45b; 18a+46b; 18a+47b; 18a+48b; 18a+49b; 18a+50b; 18a+51b; 18a+52b; 18a+53b; 18a+54b; 18a+55b; 18a+55b; 18a+57b; 18a+58b; 18a+59b; 18a+60b; 18a+61b; 18a+62b; 18a+63b; 18a+64b; 18a+65b; 18a+66b; 18a+67b; 18a+68b; 18a+69b; 18a+70b; 18a+71b; 18a+72b; 18a+73b; 18a+74b; 18a+75b; 18a+76b; 18a+77b; 18a+78b; 18a+79b; 18a+80b; 18a+81b; 18a+82b; 18a+83b; 18a+84b; 18a+85b; 18a+86b; 18a+87b; 18a+88b; 18a+89b; 18a+90b; 18a+91b; 18a+92b; 18a+93b; 18a+94b; 18a+95b; 18a+96b; 18a+97b; 19a+1b; 19a+2b; 19a+3b; 19a+4b; 19a+5b; 19a+6b; 19a+7b; 19a+8b; 19a+9b; 19a+10b; 19a+11b; 19a+12b; 19a+13b; 19a+14b; 19a+15b; 19a+16b; 19a+17b; 19a+18b; 19a+19b; 19a+20b; 19a+21b; 19a+22b; 19a+23b; 19a+24b; 19a+25b; 19a+26b; 19a+27b; 19a+28b; 19a+29b; 19a+30b; 19a+31b; 19a+32b; 19a+33b; 19a+34b; 19a+35b; 19a+36b; 19a+37b; 19a+38b; 19a+39b; 19a+40b; 19a+41b; 19a+42b; 19a+43b; 19a+44b; 19a+45b; 19a+46b; 19a+47b; 19a+48b; 19a+49b; 19a+50b; 19a+51b; 19a+52b; 19a+53b; 19a+54b; 19a+55b; 19a+55b; 19a+57b; 19a+58b; 19a+59b; 19a+60b; 19a+61b; 19a+62b; 19a+63b; 19a+64b; 19a+65b; 19a+66b; 19a+67b; 19a+68b; 19a+69b; 19a+70b; 19a+71b; 19a+72b; 19a+73b; 19a+74b; 19a+75b; 19a+76b; 19a+77b; 19a+78b; 19a+79b; 19a+80b; 19a+81b; 19a+82b; 19a+83b; 19a+84b; 19a+85b; 19a+86b; 19a+87b; 19a+88b; 19a+89b; 19a+90b; 19a+91b; 19a+92b; 19a+93b; 19a+94b; 19a+95b; 19a+96b; 19a+97b; 20a+1b; 20a+2b; 20a+3b; 20a+4b; 20a+5b; 20a+6b; 20a+7b; 20a+8b; 20a+9b; 20a+10b; 20a+11b; 20a+12b; 20a+13b; 20a+14b; 20a+15b; 20a+16b; 20a+17b; 20a+18b; 20a+19b; 20a+20b; 20a+21b; 20a+22b; 20a+23b; 20a+24b; 20a+25b; 20a+26b; 20a+27b; 20a+28b; 20a+29b; 20a+30b; 20a+31b; 20a+32b; 20a+33b; 20a+34b; 20a+35b; 20a+36b; 20a+37b; 20a+38b; 20a+39b; 20a+40b; 20a+41b; 20a+42b; 20a+43b; 20a+44b; 20a+45b; 20a+46b; 20a+47b; 20a+48b; 20a+49b; 20a+50b; 20a+51b; 20a+52b; 20a+53b; 20a+54b; 20a+55b; 20a+55b; 20a+57b; 20a+58b; 20a+59b; 20a+60b; 20a+61b; 20a+62b; 20a+63b; 20a+64b; 20a+65b; 20a+66b; 20a+67b; 20a+68b; 20a+69b; 20a+70b; 20a+71b; 20a+72b; 20a+73b; 20a+74b; 20a+75b; 20a+76b; 20a+77b; 20a+78b; 20a+79b; 20a+80b; 20a+81b; 20a+82b; 20a+83b; 20a+84b; 20a+85b; 20a+86b; 20a+87b; 20a+88b; 20a+89b; 20a+90b; 20a+91b; 20a+92b; 20a+93b; 20a+94b; 20a+95b; 20a+96b; 20a+97b; 21a+1b; 21a+2b; 21a+3b; 21a+4b; 21a+5b; 21a+6b; 21a+7b; 21a+8b; 21a+9b; 21a+10b; 21a+11b; 21a+12b; 21a+13b; 21a+14b; 21a+15b; 21a+16b; 21a+17b; 21a+18b; 21a+19b; 21a+20b; 21a+21b; 21a+22b; 21a+23b; 21a+24b; 21a+25b; 21a+26b; 21a+27b; 21a+28b; 21a+29b; 21a+30b; 21a+31b; 21a+32b; 21a+33b; 21a+34b; 21a+35b; 21a+36b; 21a+37b; 21a+38b; 21a+39b; 21a+40b; 21a+41b; 21a+42b; 21a+43b; 21a+44b; 21a+45b; 21a+46b; 21a+47b; 21a+48b; 21a+49b; 21a+50b; 21a+51b; 21a+52b; 21a+53b; 21a+54b; 21a+55b; 21a+55b; 21a+57b; 21a+58b; 21a+59b; 21a+60b; 21a+61b; 21a+62b; 21a+63b; 21a+64b; 21a+65b; 21a+66b; 21a+67b; 21a+68b; 21a+69b; 21a+70b; 21a+71b; 21a+72b; 21a+73b; 21a+74b; 21a+75b; 21a+76b; 21a+77b; 21a+78b; 21a+79b; 21a+80b; 21a+81b; 21a+82b; 21a+83b; 21a+84b; 21a+85b; 21a+86b; 21a+87b; 21a+88b; 21a+89b; 21a+90b; 21a+91b; 21a+92b; 21a+93b; 21a+94b; 21a+95b; 21a+96b; 21a+97b; 22a+1b; 22a+2b; 22a+3b; 22a+4b; 22a+5b; 22a+6b; 22a+7b; 22a+8b; 22a+9b; 22a+10b; 22a+11b; 22a+12b; 22a+13b; 22a+14b; 22a+15b; 22a+16b; 22a+17b; 22a+18b; 22a+19b; 22a+20b; 22a+21b; 22a+22b; 22a+23b; 22a+24b; 22a+25b; 22a+26b; 22a+27b; 22a+28b; 22a+29b; 22a+30b; 22a+31b; 22a+32b; 22a+33b; 22a+34b; 22a+35b; 22a+36b; 22a+37b; 22a+38b; 22a+39b; 22a+40b; 22a+41b; 22a+42b; 22a+43b; 22a+44b; 22a+45b; 22a+46b; 22a+47b; 22a+48b; 22a+49b; 22a+50b; 22a+51b; 22a+52b; 22a+53b; 22a+54b; 22a+55b; 22a+55b; 22a+57b; 22a+58b; 22a+59b; 22a+60b; 22a+61b; 22a+62b; 22a+63b; 22a+64b; 22a+65b; 22a+66b; 22a+67b; 22a+68b; 22a+69b; 22a+70b; 22a+71b; 22a+72b; 22a+73b; 22a+74b; 22a+75b; 22a+76b; 22a+77b; 22a+78b; 22a+79b; 22a+80b; 22a+81b; 22a+82b; 22a+83b; 22a+84b; 22a+85b; 22a+86b; 22a+87b; 22a+88b; 22a+89b; 22a+90b; 22a+91b; 22a+92b; 22a+93b; 22a+94b; 22a+95b; 22a+96b; 22a+97b; 23a+1b; 23a+2b; 23a+3b; 23a+4b; 23a+5b; 23a+6b; 23a+7b; 23a+8b; 23a+9b; 23a+10b; 23a+11b; 23a+12b; 23a+13b; 23a+14b; 23a+15b; 23a+16b; 23a+17b; 23a+18b; 23a+19b; 23a+20b; 23a+21b; 23a+22b; 23a+23b; 23a+24b; 23a+25b; 23a+26b; 23a+27b; 23a+28b; 23a+29b; 23a+30b; 23a+31b; 23a+32b; 23a+33b; 23a+34b; 23a+35b; 23a+36b; 23a+37b; 23a+38b; 23a+39b; 23a+40b; 23a+41b; 23a+42b; 23a+43b; 23a+44b; 23a+45b; 23a+46b; 23a+47b; 23a+48b; 23a+49b; 23a+50b; 23a+51b; 23a+52b; 23a+53b; 23a+54b; 23a+55b; 23a+55b; 23a+57b; 23a+58b; 23a+59b; 23a+60b; 23a+61b; 23a+62b; 23a+63b; 23a+64b; 23a+65b; 23a+66b; 23a+67b; 23a+68b; 23a+69b; 23a+70b; 23a+71b; 23a+72b; 23a+73b; 23a+74b; 23a+75b; 23a+76b; 23a+77b; 23a+78b; 23a+79b; 23a+80b; 23a+81b; 23a+82b; 23a+83b; 23a+84b; 23a+85b; 23a+86b; 23a+87b; 23a+88b; 23a+89b; 23a+90b; 23a+91b; 23a+92b; 23a+93b; 23a+94b; 23a+95b; 23a+96b; 23a+97b; 24a+1b; 24a+2b; 24a+3b; 24a+4b; 24a+5b; 24a+6b; 24a+7b; 24a+8b; 24a+9b; 24a+10b; 24a+11b; 24a+12b; 24a+13b; 24a+14b; 24a+15b; 24a+16b; 24a+17b; 24a+18b; 24a+19b; 24a+20b; 24a+21b; 24a+22b; 24a+23b; 24a+24b; 24a+25b; 24a+26b; 24a+27b; 24a+28b; 24a+29b; 24a+30b; 24a+31b; 24a+32b; 24a+33b; 24a+34b; 24a+35b; 24a+36b; 24a+37b; 24a+38b; 24a+39b; 24a+40b; 24a+41b; 24a+42b; 24a+43b; 24a+44b; 24a+45b; 24a+46b; 24a+47b; 24a+48b; 24a+49b; 24a+50b; 24a+51b; 24a+52b; 24a+53b; 24a+54b; 24a+55b; 24a+55b; 24a+57b; 24a+58b; 24a+59b; 24a+60b; 24a+61b; 24a+62b; 24a+63b; 24a+64b; 24a+65b; 24a+66b; 24a+67b; 24a+68b; 24a+69b; 24a+70b; 24a+71b; 24a+72b; 24a+73b; 24a+74b; 24a+75b; 24a+76b; 24a+77b; 24a+78b; 24a+79b; 24a+80b; 24a+81b; 24a+82b; 24a+83b; 24a+84b; 24a+85b; 24a+86b; 24a+87b; 24a+88b; 24a+89b; 24a+90b; 24a+91b; 24a+92b; 24a+93b; 24a+94b; 24a+95b; 24a+96b; 24a+97b; 25a+1b; 25a+2b; 25a+3b; 25a+4b; 25a+5b; 25a+6b; 25a+7b; 25a+8b; 25a+9b; 25a+10b; 25a+11b; 25a+12b; 25a+13b; 25a+14b; 25a+15b; 25a+16b; 25a+17b; 25a+18b; 25a+19b; 25a+20b; 25a+21b; 25a+22b; 25a+23b; 25a+24b; 25a+25b; 25a+26b; 25a+27b; 25a+28b; 25a+29b; 25a+30b; 25a+31b; 25a+32b; 25a+33b; 25a+34b; 25a+35b; 25a+36b; 25a+37b; 25a+38b; 25a+39b; 25a+40b; 25a+41b; 25a+42b; 25a+43b; 25a+44b; 25a+45b; 25a+46b; 25a+47b; 25a+48b; 25a+49b; 25a+50b; 25a+51b; 25a+52b; 25a+53b; 25a+54b; 25a+55b; 25a+55b; 25a+57b; 25a+58b; 25a+59b; 25a+60b; 25a+61b; 25a+62b; 25a+63b; 25a+64b; 25a+65b; 25a+66b; 25a+67b; 25a+68b; 25a+69b; 25a+70b; 25a+71b; 25a+72b; 25a+73b; 25a+74b; 25a+75b; 25a+76b; 25a+77b; 25a+78b; 25a+79b; 25a+80b; 25a+81b; 25a+82b; 25a+83b; 25a+84b; 25a+85b; 25a+86b; 25a+87b; 25a+88b; 25a+89b; 25a+90b; 25a+91b; 25a+92b; 25a+93b; 25a+94b; 25a+95b; 25a+96b; 25a+97b; 26a+1b; 26a+2b; 26a+3b; 26a+4b; 26a+5b; 26a+6b; 26a+7b; 26a+8b; 26a+9b; 26a+10b; 26a+11b; 26a+12b; 26a+13b; 26a+14b; 26a+15b; 26a+16b; 26a+17b; 26a+18b; 26a+19b; 26a+20b; 26a+21b; 26a+22b; 26a+23b; 26a+24b; 26a+25b; 26a+26b; 26a+27b; 26a+28b; 26a+29b; 26a+30b; 26a+31b; 26a+32b; 26a+33b; 26a+34b; 26a+35b; 26a+36b; 26a+37b; 26a+38b; 26a+39b; 26a+40b; 26a+41b; 26a+42b; 26a+43b; 26a+44b; 26a+45b; 26a+46b; 26a+47b; 26a+48b; 26a+49b; 26a+50b; 26a+51b; 26a+52b; 26a+53b; 26a+54b; 26a+55b; 26a+55b; 26a+57b; 26a+58b; 26a+59b; 26a+60b; 26a+61b; 26a+62b; 26a+63b; 26a+64b; 26a+65b; 26a+66b; 26a+67b; 26a+68b; 26a+69b; 26a+70b; 26a+71b; 26a+72b; 26a+73b; 26a+74b; 26a+75b; 26a+76b; 26a+77b; 26a+78b; 26a+79b; 26a+80b; 26a+81b; 26a+82b; 26a+83b; 26a+84b; 26a+85b; 26a+86b; 26a+87b; 26a+88b; 26a+89b; 26a+90b; 26a+91b; 26a+92b; 26a+93b; 26a+94b; 26a+95b; 26a+96b; 26a+97b; 26a+1b; 27a+2b; 27a+3b; 27a+4b; 27a+5b; 27a+6b; 27a+7b; 27a+8b; 27a+9b; 27a+10b; 27a+11b; 27a+12b; 27a+13b; 27a+14b; 27a+15b; 27a+16b; 27a+17b; 27a+18b; 27a+19b; 27a+20b; 27a+21b; 27a+22b; 27a+23b; 27a+24b; 27a+25b; 27a+26b; 27a+27b; 27a+28b; 27a+29b; 27a+30b; 27a+31b; 27a+32b; 27a+33b; 27a+34b; 27a+35b; 27a+36b; 27a+37b; 27a+38b; 27a+39b; 27a+40b; 27a+41b; 27a+42b; 27a+43b; 27a+44b; 27a+45b; 27a+46b; 27a+47b; 27a+48b; 27a+49b; 27a+50b; 27a+51b; 27a+52b; 27a+53b; 27a+54b; 27a+55b; 27a+55b; 27a+57b; 27a+58b; 27a+59b; 27a+60b; 27a+61b; 27a+62b; 27a+63b; 27a+64b; 27a+65b; 27a+66b; 27a+67b; 27a+68b; 27a+69b; 27a+70b; 27a+71b; 27a+72b; 27a+73b; 27a+74b; 27a+75b; 27a+76b; 27a+77b; 27a+78b; 27a+79b; 27a+80b; 27a+81b; 27a+82b; 27a+83b; 27a+84b; 27a+85b; 27a+86b; 27a+87b; 27a+88b; 27a+89b; 27a+90b; 27a+91b; 27a+92b; 27a+93b; 27a+94b; 27a+95b; 27a+96b; 27a+97b; 28a+1b; 28a+2b; 28a+3b; 28a+4b; 28a+5b; 28a+6b; 28a+7b; 28a+8b; 28a+9b; 28a+10b; 28a+11b; 28a+12b; 28a+13b; 28a+14b; 28a+15b; 28a+16b; 28a+17b; 28a+18b; 28a+19b; 28a+20b; 28a+21b; 28a+22b; 28a+23b; 28a+24b; 28a+25b; 28a+26b; 28a+27b; 28a+28b; 28a+29b; 28a+30b; 28a+31b; 28a+32b; 28a+33b; 28a+34b; 28a+35b; 28a+36b; 28a+37b; 28a+38b; 28a+39b; 28a+40b; 28a+41b; 28a+42b; 28a+43b; 28a+44b; 28a+45b; 28a+46b; 28a+47b; 28a+48b; 28a+49b; 28a+50b; 28a+51b; 28a+52b; 28a+53b; 28a+54b; 28a+55b; 28a+55b; 28a+57b; 28a+58b; 28a+59b; 28a+60b; 28a+61b; 28a+62b; 28a+63b; 28a+64b; 28a+65b; 28a+66b; 28a+67b; 28a+68b; 28a+69b; 28a+70b; 28a+71b; 28a+72b; 28a+73b; 28a+74b; 28a+75b; 28a+76b; 28a+77b; 28a+78b; 28a+79b; 28a+80b; 28a+81b; 28a+82b; 28a+83b; 28a+84b; 28a+85b; 28a+86b; 28a+87b; 28a+88b; 28a+89b; 28a+90b; 28a+91b; 28a+92b; 28a+93b; 28a+94b; 28a+95b; 28a+96b; 28a+97b; 29a+1b; 29a+2b; 29a+3b; 29a+4b; 29a+5b; 29a+6b; 29a+7b; 29a+8b; 29a+9b; 29a+10b; 29a+11b; 29a+12b; 29a+13b; 29a+14b; 29a+15b; 29a+16b; 29a+17b; 29a+18b; 29a+19b; 29a+20b; 29a+21b; 29a+22b; 29a+23b; 29a+24b; 29a+25b; 29a+26b; 29a+27b; 29a+28b; 29a+29b; 29a+30b; 29a+31b; 29a+32b; 29a+33b; 29a+34b; 29a+35b; 29a+36b; 29a+37b; 29a+38b; 29a+39b; 29a+40b; 29a+41b; 29a+42b; 29a+43b; 29a+44b; 29a+45b; 29a+46b; 29a+47b; 29a+48b; 29a+49b; 29a+50b; 29a+51b; 29a+52b; 29a+53b; 29a+54b; 29a+55b; 29a+55b; 29a+57b; 29a+58b; 29a+59b; 29a+60b; 29a+61b; 29a+62b; 29a+63b; 29a+64b; 29a+65b; 29a+66b; 29a+67b; 29a+68b; 29a+69b; 29a+70b; 29a+71b; 29a+72b; 29a+73b; 29a+74b; 29a+75b; 29a+76b; 29a+77b; 29a+78b; 29a+79b; 29a+80b; 29a+81b; 29a+82b; 29a+83b; 29a+84b; 29a+85b; 29a+86b; 29a+87b; 29a+88b; 29a+89b; 29a+90b; 29a+91b; 29a+92b; 29a+93b; 29a+94b; 29a+95b; 29a+96b; 29a+97b; 30a+1b; 30a+2b; 30a+3b; 30a+4b; 30a+5b; 30a+6b; 30a+7b; 30a+8b; 30a+9b; 30a+10b; 30a+11b; 30a+12b; 30a+13b; 30a+14b; 30a+15b; 30a+16b; 30a+17b; 30a+18b; 30a+19b; 30a+20b; 30a+21b; 30a+22b; 30a+23b; 30a+24b; 30a+25b; 30a+26b; 30a+27b; 30a+28b; 30a+29b; 30a+30b; 30a+31b; 30a+32b; 30a+33b; 30a+34b; 30a+35b; 30a+36b; 30a+37b; 30a+38b; 30a+39b; 30a+40b; 30a+41b; 30a+42b; 30a+43b; 30a+44b; 30a+45b; 30a+46b; 30a+47b; 30a+48b; 30a+49b; 30a+50b; 30a+51b; 30a+52b; 30a+53b; 30a+54b; 30a+55b; 30a+55b; 30a+57b; 30a+58b; 30a+59b; 30a+60b; 30a+61b; 30a+62b; 30a+63b; 30a+64b; 30a+65b; 30a+66b; 30a+67b; 30a+68b; 30a+69b; 30a+70b; 30a+71b; 30a+72b; 30a+73b; 30a+74b; 30a+75b; 30a+76b; 30a+77b; 30a+78b; 30a+79b; 30a+80b; 30a+81b; 30a+82b; 30a+83b; 30a+84b; 30a+85b; 30a+86b; 30a+87b; 30a+88b; 30a+89b; 30a+90b; 30a+91b; 30a+92b; 30a+93b; 30a+94b; 30a+95b; 30a+96b; 30a+97b; 31a+1b; 31a+2b; 31a+3b; 31a+4b; 31a+5b; 31a+6b; 31a+7b; 31a+8b; 31a+9b; 31a+10b; 31a+11b; 31a+12b; 31a+13b; 31a+14b; 31a+15b; 31a+16b; 31a+17b; 31a+18b; 31a+19b; 31a+20b; 31a+21b; 31a+22b; 31a+23b; 31a+24b; 31a+25b; 31a+26b; 31a+27b; 31a+28b; 31a+29b; 31a+30b; 31a+31b; 31a+32b; 31a+33b; 31a+34b; 31a+35b; 31a+36b; 31a+37b; 31a+38b; 31a+39b; 31a+40b; 31a+41b; 31a+42b; 31a+43b; 31a+44b; 31a+45b; 31a+46b; 31a+47b; 31a+48b; 31a+49b; 31a+50b; 31a+51b; 31a+52b; 31a+53b; 31a+54b; 31a+55b; 31a+55b; 31a+57b; 31a+58b; 31a+59b; 31a+60b; 31a+61b; 31a+62b; 31a+63b; 31a+64b; 31a+65b; 31a+66b; 31a+67b; 31a+68b; 31a+69b; 31a+70b; 31a+71b; 31a+72b; 31a+73b; 31a+74b; 31a+75b; 31a+76b; 31a+77b; 31a+78b; 31a+79b; 31a+80b; 31a+81b; 31a+82b; 31a+83b; 31a+84b; 31a+85b; 31a+86b; 31a+87b; 31a+88b; 31a+89b; 31a+90b; 31a+91b; 31a+92b; 31a+93b; 31a+94b; 31a+95b; 31a+96b; 31a+97b; 32a+1b; 32a+2b; 32a+3b; 32a+4b; 32a+5b; 32a+6b; 32a+7b; 32a+8b; 32a+9b; 32a+10b; 32a+11b; 32a+12b; 32a+13b; 32a+14b; 32a+15b; 32a+16b; 32a+17b; 32a+18b; 32a+19b; 32a+20b; 32a+21b; 32a+22b; 32a+23b; 32a+24b; 32a+25b; 32a+26b; 32a+27b; 32a+28b; 32a+29b; 32a+30b; 32a+31b; 32a+32b; 32a+33b; 32a+34b; 32a+35b; 32a+36b; 32a+37b; 32a+38b; 32a+39b; 32a+40b; 32a+41b; 32a+42b; 32a+43b; 32a+44b; 32a+45b; 32a+46b; 32a+47b; 32a+48b; 32a+49b; 32a+50b; 32a+51b; 32a+52b; 32a+53b; 32a+54b; 32a+55b; 32a+55b; 32a+57b; 32a+58b; 32a+59b; 32a+60b; 32a+61b; 32a+62b; 32a+63b; 32a+64b; 32a+65b; 32a+66b; 32a+67b; 32a+68b; 32a+69b; 32a+70b; 32a+71b; 32a+72b; 32a+73b; 32a+74b; 32a+75b; 32a+76b; 32a+77b; 32a+78b; 32a+79b; 32a+80b; 32a+81b; 32a+82b; 32a+83b; 32a+84b; 32a+85b; 32a+86b; 32a+87b; 32a+88b; 32a+89b; 32a+90b; 32a+91b; 32a+92b; 32a+93b; 32a+94b; 32a+95b; 32a+96b; 32a+97b; 33a+1b; 33a+2b; 33a+3b; 33a+4b; 33a+5b; 33a+6b; 33a+7b; 33a+8b; 33a+9b; 33a+10b; 33a+11b; 33a+12b; 33a+13b; 33a+14b; 33a+15b; 33a+16b; 33a+17b; 33a+18b; 33a+19b; 33a+20b; 33a+21b; 33a+22b; 33a+23b; 33a+24b; 33a+25b; 33a+26b; 33a+27b; 33a+28b; 33a+29b; 33a+30b; 33a+31b; 33a+32b; 33a+33b; 33a+34b; 33a+35b; 33a+36b; 33a+37b; 33a+38b; 33a+39b; 33a+40b; 33a+41b; 33a+42b; 33a+43b; 33a+44b; 33a+45b; 33a+46b; 33a+47b; 33a+48b; 33a+49b; 33a+50b; 33a+51b; 33a+52b; 33a+53b; 33a+54b; 33a+55b; 33a+55b; 33a+57b; 33a+58b; 33a+59b; 33a+60b; 33a+61b; 33a+62b; 33a+63b; 33a+64b; 33a+65b; 33a+66b; 33a+67b; 33a+68b; 33a+69b; 33a+70b; 33a+71b; 33a+72b; 33a+73b; 33a+74b; 33a+75b; 33a+76b; 33a+77b; 33a+78b; 33a+79b; 33a+80b; 33a+81b; 33a+82b; 33a+83b; 33a+84b; 33a+85b; 33a+86b; 33a+87b; 33a+88b; 33a+89b; 33a+90b; 33a+91b; 33a+92b; 33a+93b; 33a+94b; 33a+95b; 33a+96b; 33a+97b; 34a+1b; 34a+2b; 34a+3b; 34a+4b; 34a+5b; 34a+6b; 34a+7b; 34a+8b; 34a+9b; 34a+10b; 34a+11b; 34a+12b; 34a+13b; 34a+14b; 34a+15b; 34a+16b; 34a+17b; 34a+18b; 34a+19b; 34a+20b; 34a+21b; 34a+22b; 34a+23b; 34a+24b; 34a+25b; 34a+26b; 34a+27b; 34a+28b; 34a+29b; 34a+30b; 34a+31b; 34a+32b; 34a+33b; 34a+34b; 34a+35b; 34a+36b; 34a+37b; 34a+38b; 34a+39b; 34a+40b; 34a+41b; 34a+42b; 34a+43b; 34a+44b; 34a+45b; 34a+46b; 34a+47b; 34a+48b; 34a+49b; 34a+50b; 34a+51b; 34a+52b; 34a+53b; 34a+54b; 34a+55b; 34a+55b; 34a+57b; 34a+58b; 34a+59b; 34a+60b; 34a+61b; 34a+62b; 34a+63b; 34a+64b; 34a+65b; 34a+66b; 34a+67b; 34a+68b; 34a+69b; 34a+70b; 34a+71b; 34a+72b; 34a+73b; 34a+74b; 34a+75b; 34a+76b; 34a+77b; 34a+78b; 34a+79b; 34a+80b; 34a+81b; 34a+82b; 34a+83b; 34a+84b; 34a+85b; 34a+86b; 34a+87b; 34a+88b; 34a+89b; 34a+90b; 34a+91b; 34a+92b; 34a+93b; 34a+94b; 34a+95b; 34a+96b; 34a+97b; 35a+1b; 35a+2b; 35a+3b; 35a+4b; 35a+5b; 35a+6b; 35a+7b; 35a+8b; 35a+9b; 35a+10b; 35a+11b; 35a+12b; 35a+13b; 35a+14b; 35a+15b; 35a+16b; 35a+17b; 35a+18b; 35a+19b; 35a+20b; 35a+21b; 35a+22b; 35a+23b; 35a+24b; 35a+25b; 35a+26b; 35a+27b; 35a+28b; 35a+29b; 35a+30b; 35a+31b; 35a+32b; 35a+33b; 35a+34b; 35a+35b; 35a+36b; 35a+37b; 35a+38b; 35a+39b; 35a+40b; 35a+41b; 35a+42b; 35a+43b; 35a+44b; 35a+45b; 35a+46b; 35a+47b; 35a+48b; 35a+49b; 35a+50b; 35a+51b; 35a+52b; 35a+53b; 35a+54b; 35a+55b; 35a+55b; 35a+57b; 35a+58b; 35a+59b; 35a+60b; 35a+61b; 35a+62b; 35a+63b; 35a+64b; 35a+65b; 35a+66b; 35a+67b; 35a+68b; 35a+69b; 35a+70b; 35a+71b; 35a+72b; 35a+73b; 35a+74b; 35a+75b; 35a+76b; 35a+77b; 35a+78b; 35a+79b; 35a+80b; 35a+81b; 35a+82b; 35a+83b; 35a+84b; 35a+85b; 35a+86b; 35a+87b; 35a+88b; 35a+89b; 35a+90b; 35a+91b; 35a+92b; 35a+93b; 35a+94b; 35a+95b; 35a+96b; 35a+97b; 36a+1b; 36a+2b; 36a+3b; 36a+4b; 36a+5b; 36a+6b; 36a+7b; 36a+8b; 36a+9b; 36a+10b; 36a+11b; 36a+12b; 36a+13b; 36a+14b; 36a+15b; 36a+16b; 36a+17b; 36a+18b; 36a+19b; 36a+20b; 36a+21b; 36a+22b; 36a+23b; 36a+24b; 36a+25b; 36a+26b; 36a+27b; 36a+28b; 36a+29b; 36a+30b; 36a+31b; 36a+32b; 36a+33b; 36a+34b; 36a+35b; 36a+36b; 36a+37b; 36a+38b; 36a+39b; 36a+40b; 36a+41b; 36a+42b; 36a+43b; 36a+44b; 36a+45b; 36a+46b; 36a+47b; 36a+48b; 36a+49b; 36a+50b; 36a+51b; 36a+52b; 36a+53b; 36a+54b; 36a+55b; 36a+55b; 36a+57b; 36a+58b; 36a+59b; 36a+60b; 36a+61b; 36a+62b; 36a+63b; 36a+64b; 36a+65b; 36a+66b; 36a+67b; 36a+68b; 36a+69b; 36a+70b; 36a+71b; 36a+72b; 36a+73b; 36a+74b; 36a+75b; 36a+76b; 36a+77b; 36a+78b; 36a+79b; 36a+80b; 36a+81b; 36a+82b; 36a+83b; 36a+84b; 36a+85b; 36a+86b; 36a+87b; 36a+88b; 36a+89b; 36a+90b; 36a+91b; 36a+92b; 36a+93b; 36a+94b; 36a+95b; 36a+96b; 36a+97b; 36a+1b; 37a+2b; 37a+3b; 37a+4b; 37a+5b; 37a+6b; 37a+7b; 37a+8b; 37a+9b; 37a+10b; 37a+11b; 37a+12b; 37a+13b; 37a+14b; 37a+15b; 37a+16b; 37a+17b; 37a+18b; 37a+19b; 37a+20b; 37a+21b; 37a+22b; 37a+23b; 37a+24b; 37a+25b; 37a+26b; 37a+27b; 37a+28b; 37a+29b; 37a+30b; 37a+31b; 37a+32b; 37a+33b; 37a+34b; 37a+35b; 37a+36b; 37a+37b; 37a+38b; 37a+39b; 37a+40b; 37a+41b; 37a+42b; 37a+43b; 37a+44b; 37a+45b; 37a+46b; 37a+47b; 37a+48b; 37a+49b; 37a+50b; 37a+51b; 37a+52b; 37a+53b; 37a+54b; 37a+55b; 37a+55b; 37a+57b; 37a+58b; 37a+59b; 37a+60b; 37a+61b; 37a+62b; 37a+63b; 37a+64b; 37a+65b; 37a+66b; 37a+67b; 37a+68b; 37a+69b; 37a+70b; 37a+71b; 37a+72b; 37a+73b; 37a+74b; 37a+75b; 37a+76b; 37a+77b; 37a+78b; 37a+79b; 37a+80b; 37a+81b; 37a+82b; 37a+83b; 37a+84b; 37a+85b; 37a+86b; 37a+87b; 37a+88b; 37a+89b; 37a+90b; 37a+91b; 37a+92b; 37a+93b; 37a+94b; 37a+95b; 37a+96b; 37a+97b; 38a+1b; 38a+2b; 38a+3b; 38a+4b; 38a+5b; 38a+6b; 38a+7b; 38a+8b; 38a+9b; 38a+10b; 38a+11b; 38a+12b; 38a+13b; 38a+14b; 38a+15b; 38a+16b; 38a+17b; 38a+18b; 38a+19b; 38a+20b; 38a+21b; 38a+22b; 38a+23b; 38a+24b; 38a+25b; 38a+26b; 38a+27b; 38a+28b; 38a+29b; 38a+30b; 38a+31b; 38a+32b; 38a+33b; 38a+34b; 38a+35b; 38a+36b; 38a+37b; 38a+38b; 38a+39b; 38a+40b; 38a+41b; 38a+42b; 38a+43b; 38a+44b; 38a+45b; 38a+46b; 38a+47b; 38a+48b; 38a+49b; 38a+50b; 38a+51b; 38a+52b; 38a+53b; 38a+54b; 38a+55b; 38a+55b; 38a+57b; 38a+58b; 38a+59b; 38a+60b; 38a+61b; 38a+62b; 38a+63b; 38a+64b; 38a+65b; 38a+66b; 38a+67b; 38a+68b; 38a+69b; 38a+70b; 38a+71b; 38a+72b; 38a+73b; 38a+74b; 38a+75b; 38a+76b; 38a+77b; 38a+78b; 38a+79b; 38a+80b; 38a+81b; 38a+82b; 38a+83b; 38a+84b; 38a+85b; 38a+86b; 38a+87b; 38a+88b; 38a+89b; 38a+90b; 38a+91b; 38a+92b; 38a+93b; 38a+94b; 38a+95b; 38a+96b; 38a+97b; 39a+1b; 39a+2b; 39a+3b; 39a+4b; 39a+5b; 39a+6b; 39a+7b; 39a+8b; 39a+9b; 39a+10b; 39a+11b; 39a+12b; 39a+13b; 39a+14b; 39a+15b; 39a+16b; 39a+17b; 39a+18b; 39a+19b; 39a+20b; 39a+21b; 39a+22b; 39a+23b; 39a+24b; 39a+25b; 39a+26b; 39a+27b; 39a+28b; 39a+29b; 39a+30b; 39a+31b; 39a+32b; 39a+33b; 39a+34b; 39a+35b; 39a+36b; 39a+37b; 39a+38b; 39a+39b; 39a+40b; 39a+41b; 39a+42b; 39a+43b; 39a+44b; 39a+45b; 39a+46b; 39a+47b; 39a+48b; 39a+49b; 39a+50b; 39a+51b; 39a+52b; 39a+53b; 39a+54b; 39a+55b; 39a+55b; 39a+57b; 39a+58b; 39a+59b; 39a+60b; 39a+61b; 39a+62b; 39a+63b; 39a+64b; 39a+65b; 39a+66b; 39a+67b; 39a+68b; 39a+69b; 39a+70b; 39a+71b; 39a+72b; 39a+73b; 39a+74b; 39a+75b; 39a+76b; 39a+77b; 39a+78b; 39a+79b; 39a+80b; 39a+81b; 39a+82b; 39a+83b; 39a+84b; 39a+85b; 39a+86b; 39a+87b; 39a+88b; 39a+89b; 39a+90b; 39a+91b; 39a+92b; 39a+93b; 39a+94b; 39a+95b; 39a+96b; 39a+97b; 40a+1b; 40a+2b; 40a+3b; 40a+4b; 40a+5b; 40a+6b; 40a+7b; 40a+8b; 40a+9b; 40a+10b; 40a+11b; 40a+12b; 40a+13b; 40a+14b; 40a+15b; 40a+16b; 40a+17b; 40a+18b; 40a+19b; 40a+20b; 40a+21b; 40a+22b; 40a+23b; 40a+24b; 40a+25b; 40a+26b; 40a+27b; 40a+28b; 40a+29b; 40a+30b; 40a+31b; 40a+32b; 40a+33b; 40a+34b; 40a+35b; 40a+36b; 40a+37b; 40a+38b; 40a+39b; 40a+40b; 40a+41b; 40a+42b; 40a+43b; 40a+44b; 40a+45b; 40a+46b; 40a+47b; 40a+48b; 40a+49b; 40a+50b; 40a+51b; 40a+52b; 40a+53b; 40a+54b; 40a+55b; 40a+55b; 40a+57b; 40a+58b; 40a+59b; 40a+60b; 40a+61b; 40a+62b; 40a+63b; 40a+64b; 40a+65b; 40a+66b; 40a+67b; 40a+68b; 40a+69b; 40a+70b; 40a+71b; 40a+72b; 40a+73b; 40a+74b; 40a+75b; 40a+76b; 40a+77b; 40a+78b; 40a+79b; 40a+80b; 40a+81b; 40a+82b; 40a+83b; 40a+84b; 40a+85b; 40a+86b; 40a+87b; 40a+88b; 40a+89b; 40a+90b; 40a+91b; 40a+92b; 40a+93b; 40a+94b; 40a+95b; 40a+96b; 40a+97b; 41a+1b; 41a+2b; 41a+3b; 41a+4b; 41a+5b; 41a+6b; 41a+7b; 41a+8b; 41a+9b; 41a+10b; 41a+11b; 41a+12b; 41a+13b; 41a+14b; 41a+15b; 41a+16b; 41a+17b; 41a+18b; 41a+19b; 41a+20b; 41a+21b; 41a+22b; 41a+23b; 41a+24b; 41a+25b; 41a+26b; 41a+27b; 41a+28b; 41a+29b; 41a+30b; 41a+31b; 41a+32b; 41a+33b; 41a+34b; 41a+35b; 41a+36b; 41a+37b; 41a+38b; 41a+39b; 41a+40b; 41a+41b; 41a+42b; 41a+43b; 41a+44b; 41a+45b; 41a+46b; 41a+47b; 41a+48b; 41a+49b; 41a+50b; 41a+51b; 41a+52b; 41a+53b; 41a+54b; 41a+55b; 41a+55b; 41a+57b; 41a+58b; 41a+59b; 41a+60b; 41a+61b; 41a+62b; 41a+63b; 41a+64b; 41a+65b; 41a+66b; 41a+67b; 41a+68b; 41a+69b; 41a+70b; 41a+71b; 41a+72b; 41a+73b; 41a+74b; 41a+75b; 41a+76b; 41a+77b; 41a+78b; 41a+79b; 41a+80b; 41a+81b; 41a+82b; 41a+83b; 41a+84b; 41a+85b; 41a+86b; 41a+87b; 41a+88b; 41a+89b; 41a+90b; 41a+91b; 41a+92b; 41a+93b; 41a+94b; 41a+95b; 41a+96b; 41a+97b; 42a+1b; 42a+2b; 42a+3b; 42a+4b; 42a+5b; 42a+6b; 42a+7b; 42a+8b; 42a+9b; 42a+10b; 42a+11b; 42a+12b; 42a+13b; 42a+14b; 42a+15b; 42a+16b; 42a+17b; 42a+18b; 42a+19b; 42a+20b; 42a+21b; 42a+22b; 42a+23b; 42a+24b; 42a+25b; 42a+26b; 42a+27b; 42a+28b; 42a+29b; 42a+30b; 42a+31b; 42a+32b; 42a+33b; 42a+34b; 42a+35b; 42a+36b; 42a+37b; 42a+38b; 42a+39b; 42a+40b; 42a+41b; 42a+42b; 42a+43b; 42a+44b; 42a+45b; 42a+46b; 42a+47b; 42a+48b; 42a+49b; 42a+50b; 42a+51b; 42a+52b; 42a+53b; 42a+54b; 42a+55b; 42a+55b; 42a+57b; 42a+58b; 42a+59b; 42a+60b; 42a+61b; 42a+62b; 42a+63b; 42a+64b; 42a+65b; 42a+66b; 42a+67b; 42a+68b; 42a+69b; 42a+70b; 42a+71b; 42a+72b; 42a+73b; 42a+74b; 42a+75b; 42a+76b; 42a+77b; 42a+78b; 42a+79b; 42a+80b; 42a+81b; 42a+82b; 42a+83b; 42a+84b; 42a+85b; 42a+86b; 42a+87b; 42a+88b; 42a+89b; 42a+90b; 42a+91b; 42a+92b; 42a+93b; 42a+94b; 42a+95b; 42a+96b; 42a+97b; 43a+1b; 43a+2b; 43a+3b; 43a+4b; 43a+5b; 43a+6b; 43a+7b; 43a+8b; 43a+9b; 43a+10b; 43a+11b; 43a+12b; 43a+13b; 43a+14b; 43a+15b; 43a+16b; 43a+17b; 43a+18b; 43a+19b; 43a+20b; 43a+21b; 43a+22b; 43a+23b; 43a+24b; 43a+25b; 43a+26b; 43a+27b; 43a+28b; 43a+29b; 43a+30b; 43a+31b; 43a+32b; 43a+33b; 43a+34b; 43a+35b; 43a+36b; 43a+37b; 43a+38b; 43a+39b; 43a+40b; 43a+41b; 43a+42b; 43a+43b; 43a+44b; 43a+45b; 43a+46b; 43a+47b; 43a+48b; 43a+49b; 43a+50b; 43a+51b; 43a+52b; 43a+53b; 43a+54b; 43a+55b; 43a+55b; 43a+57b; 43a+58b; 43a+59b; 43a+60b; 43a+61b; 43a+62b; 43a+63b; 43a+64b; 43a+65b; 43a+66b; 43a+67b; 43a+68b; 43a+69b; 43a+70b; 43a+71b; 43a+72b; 43a+73b; 43a+74b; 43a+75b; 43a+76b; 43a+77b; 43a+78b; 43a+79b; 43a+80b; 43a+81b; 43a+82b; 43a+83b; 43a+84b; 43a+85b; 43a+86b; 43a+87b; 43a+88b; 43a+89b; 43a+90b; 43a+91b; 43a+92b; 43a+93b; 43a+94b; 43a+95b; 43a+96b; 43a+97b; 44a+1b; 44a+2b; 44a+3b; 44a+4b; 44a+5b; 44a+6b; 44a+7b; 44a+8b; 44a+9b; 44a+10b; 44a+11b; 44a+12b; 44a+13b; 44a+14b; 44a+15b; 44a+16b; 44a+17b; 44a+18b; 44a+19b; 44a+20b; 44a+21b; 44a+22b; 44a+23b; 44a+24b; 44a+25b; 44a+26b; 44a+27b; 44a+28b; 44a+29b; 44a+30b; 44a+31b; 44a+32b; 44a+33b; 44a+34b; 44a+35b; 44a+36b; 44a+37b; 44a+38b; 44a+39b; 44a+40b; 44a+41b; 44a+42b; 44a+43b; 44a+44b; 44a+45b; 44a+46b; 44a+47b; 44a+48b; 44a+49b; 44a+50b; 44a+51b; 44a+52b; 44a+53b; 44a+54b; 44a+55b; 44a+55b; 44a+57b; 44a+58b; 44a+59b; 44a+60b; 44a+61b; 44a+62b; 44a+63b; 44a+64b; 44a+65b; 44a+66b; 44a+67b; 44a+68b; 44a+69b; 44a+70b; 44a+71b; 44a+72b; 44a+73b; 44a+74b; 44a+75b; 44a+76b; 44a+77b; 44a+78b; 44a+79b; 44a+80b; 44a+81b; 44a+82b; 44a+83b; 44a+84b; 44a+85b; 44a+86b; 44a+87b; 44a+88b; 44a+89b; 44a+90b; 44a+91b; 44a+92b; 44a+93b; 44a+94b; 44a+95b; 44a+96b; 44a+97b; 45a+1b; 45a+2b; 45a+3b; 45a+4b; 45a+5b; 45a+6b; 45a+7b; 45a+8b; 45a+9b; 45a+10b; 45a+11b; 45a+12b; 45a+13b; 45a+14b; 45a+15b; 45a+16b; 45a+17b; 45a+18b; 45a+19b; 45a+20b; 45a+21b; 45a+22b; 45a+23b; 45a+24b; 45a+25b; 45a+26b; 45a+27b; 45a+28b; 45a+29b; 45a+30b; 45a+31b; 45a+32b; 45a+33b; 45a+34b; 45a+35b; 45a+36b; 45a+37b; 45a+38b; 45a+39b; 45a+40b; 45a+41b; 45a+42b; 45a+43b; 45a+44b; 45a+45b; 45a+46b; 45a+47b; 45a+48b; 45a+49b; 45a+50b; 45a+51b; 45a+52b; 45a+53b; 45a+54b; 45a+55b; 45a+55b; 45a+57b; 45a+58b; 45a+59b; 45a+60b; 45a+61b; 45a+62b; 45a+63b; 45a+64b; 45a+65b; 45a+66b; 45a+67b; 45a+68b; 45a+69b; 45a+70b; 45a+71b; 45a+72b; 45a+73b; 45a+74b; 45a+75b; 45a+76b; 45a+77b; 45a+78b; 45a+79b; 45a+80b; 45a+81b; 45a+82b; 45a+83b; 45a+84b; 45a+85b; 45a+86b; 45a+87b; 45a+88b; 45a+89b; 45a+90b; 45a+91b; 45a+92b; 45a+93b; 45a+94b; 45a+95b; 45a+96b; 45a+97b; 46a+1b; 46a+2b; 46a+3b; 46a+4b; 46a+5b; 46a+6b; 46a+7b; 46a+8b; 46a+9b; 46a+10b; 46a+11b; 46a+12b; 46a+13b; 46a+14b; 46a+15b; 46a+16b; 46a+17b; 46a+18b; 46a+19b; 46a+20b; 46a+21b; 46a+22b; 46a+23b; 46a+24b; 46a+25b; 46a+26b; 46a+27b; 46a+28b; 46a+29b; 46a+30b; 46a+31b; 46a+32b; 46a+33b; 46a+34b; 46a+35b; 46a+36b; 46a+37b; 46a+38b; 46a+39b; 46a+40b; 46a+41b; 46a+42b; 46a+43b; 46a+44b; 46a+45b; 46a+46b; 46a+47b; 46a+48b; 46a+49b; 46a+50b; 46a+51b; 46a+52b; 46a+53b; 46a+54b; 46a+55b; 46a+55b; 46a+57b; 46a+58b; 46a+59b; 46a+60b; 46a+61b; 46a+62b; 46a+63b; 46a+64b; 46a+65b; 46a+66b; 46a+67b; 46a+68b; 46a+69b; 46a+70b; 46a+71b; 46a+72b; 46a+73b; 46a+74b; 46a+75b; 46a+76b; 46a+77b; 46a+78b; 46a+79b; 46a+80b; 46a+81b; 46a+82b; 46a+83b; 46a+84b; 46a+85b; 46a+86b; 46a+87b; 46a+88b; 46a+89b; 46a+90b; 46a+91b; 46a+92b; 46a+93b; 46a+94b; 46a+95b; 46a+96b; 46a+97b; 47a+1b; 47a+2b; 47a+3b; 47a+4b; 47a+5b; 47a+6b; 47a+7b; 47a+8b; 47a+9b; 47a+10b; 47a+11b; 47a+12b; 47a+13b; 47a+14b; 47a+15b; 47a+16b; 47a+17b; 47a+18b; 47a+19b; 47a+20b; 47a+21b; 47a+22b; 47a+23b; 47a+24b; 47a+25b; 47a+26b; 47a+27b; 47a+28b; 47a+29b; 47a+30b; 47a+31b; 47a+32b; 47a+33b; 47a+34b; 47a+35b; 47a+36b; 47a+37b; 47a+38b; 47a+39b; 47a+40b; 47a+41b; 47a+42b; 47a+43b; 47a+44b; 47a+45b; 47a+46b; 47a+47b; 47a+48b; 47a+49b; 47a+50b; 47a+51b; 47a+52b; 47a+53b; 47a+54b; 47a+55b; 47a+55b; 47a+57b; 47a+58b; 47a+59b; 47a+60b; 47a+61b; 47a+62b; 47a+63b; 47a+64b; 47a+65b; 47a+66b; 47a+67b; 47a+68b; 47a+69b; 47a+70b; 47a+71b; 47a+72b; 47a+73b; 47a+74b; 47a+75b; 47a+76b; 47a+77b; 47a+78b; 47a+79b; 47a+80b; 47a+81b; 47a+82b; 47a+83b; 47a+84b; 47a+85b; 47a+86b; 47a+87b; 47a+88b; 47a+89b; 47a+90b; 47a+91b; 47a+92b; 47a+93b; 47a+94b; 47a+95b; 47a+96b; 47a+97b; 48a+1b; 48a+2b; 48a+3b; 48a+4b; 48a+5b; 48a+6b; 48a+7b; 48a+8b; 48a+9b; 48a+10b; 48a+11b; 48a+12b; 48a+13b; 48a+14b; 48a+15b; 48a+16b; 48a+17b; 48a+18b; 48a+19b; 48a+20b; 48a+21b; 48a+22b; 48a+23b; 48a+24b; 48a+25b; 48a+26b; 48a+27b; 48a+28b; 48a+29b; 48a+30b; 48a+31b; 48a+32b; 48a+33b; 48a+34b; 48a+35b; 48a+36b; 48a+37b; 48a+38b; 48a+39b; 48a+40b; 48a+41b; 48a+42b; 48a+43b; 48a+44b; 48a+45b; 48a+46b; 48a+47b; 48a+48b; 48a+49b; 48a+50b; 48a+51b; 48a+52b; 48a+53b; 48a+54b; 48a+55b; 48a+55b; 48a+57b; 48a+58b; 48a+59b; 48a+60b; 48a+61b; 48a+62b; 48a+63b; 48a+64b; 48a+65b; 48a+66b; 48a+67b; 48a+68b; 48a+69b; 48a+70b; 48a+71b; 48a+72b; 48a+73b; 48a+74b; 48a+75b; 48a+76b; 48a+77b; 48a+78b; 48a+79b; 48a+80b; 48a+81b; 48a+82b; 48a+83b; 48a+84b; 48a+85b; 48a+86b; 48a+87b; 48a+88b; 48a+89b; 48a+90b; 48a+91b; 48a+92b; 48a+93b; 48a+94b; 48a+95b; 48a+96b; 48a+97b; 49a+1b; 49a+2b; 49a+3b; 49a+4b; 49a+5b; 49a+6b; 49a+7b; 49a+8b; 49a+9b; 49a+10b; 49a+11b; 49a+12b; 49a+13b; 49a+14b; 49a+15b; 49a+16b; 49a+17b; 49a+18b; 49a+19b; 49a+20b; 49a+21b; 49a+22b; 49a+23b; 49a+24b; 49a+25b; 49a+26b; 49a+27b; 49a+28b; 49a+29b; 49a+30b; 49a+31b; 49a+32b; 49a+33b; 49a+34b; 49a+35b; 49a+36b; 49a+37b; 49a+38b; 49a+39b; 49a+40b; 49a+41b; 49a+42b; 49a+43b; 49a+44b; 49a+45b; 49a+46b; 49a+47b; 49a+48b; 49a+49b; 49a+50b; 49a+51b; 49a+52b; 49a+53b; 49a+54b; 49a+55b; 49a+55b; 49a+57b; 49a+58b; 49a+59b; 49a+60b; 49a+61b; 49a+62b; 49a+63b; 49a+64b; 49a+65b; 49a+66b; 49a+67b; 49a+68b; 49a+69b; 49a+70b; 49a+71b; 49a+72b; 49a+73b; 49a+74b; 49a+75b; 49a+76b; 49a+77b; 49a+78b; 49a+79b; 49a+80b; 49a+81b; 49a+82b; 49a+83b; 49a+84b; 49a+85b; 49a+86b; 49a+87b; 49a+88b; 49a+89b; 49a+90b; 49a+91b; 49a+92b; 49a+93b; 49a+94b; 49a+95b; 49a+96b; 49a+97b; 50a+1b; 50a+2b; 50a+3b; 50a+4b; 50a+5b; 50a+6b; 50a+7b; 50a+8b; 50a+9b; 50a+10b; 50a+11b; 50a+12b; 50a+13b; 50a+14b; 50a+15b; 50a+16b; 50a+17b; 50a+18b; 50a+19b; 50a+20b; 50a+21b; 50a+22b; 50a+23b; 50a+24b; 50a+25b; 50a+26b; 50a+27b; 50a+28b; 50a+29b; 50a+30b; 50a+31b; 50a+32b; 50a+33b; 50a+34b; 50a+35b; 50a+36b; 50a+37b; 50a+38b; 50a+39b; 50a+40b; 50a+41b; 50a+42b; 50a+43b; 50a+44b; 50a+45b; 50a+46b; 50a+47b; 50a+48b; 50a+49b; 50a+50b; 50a+51b; 50a+52b; 50a+53b; 50a+54b; 50a+55b; 50a+55b; 50a+57b; 50a+58b; 50a+59b; 50a+60b; 50a+61b; 50a+62b; 50a+63b; 50a+64b; 50a+65b; 50a+66b; 50a+67b; 50a+68b; 50a+69b; 50a+70b; 50a+71b; 50a+72b; 50a+73b; 50a+74b; 50a+75b; 50a+76b; 50a+77b; 50a+78b; 50a+79b; 50a+80b; 50a+81b; 50a+82b; 50a+83b; 50a+84b; 50a+85b; 50a+86b; 50a+87b; 50a+88b; 50a+89b; 50a+90b; 50a+91b; 50a+92b; 50a+93b; 50a+94b; 50a+95b; 50a+96b; 50a+97b; 51a+1b; 51a+2b; 51a+3b; 51a+4b; 51a+5b; 51a+6b; 51a+7b; 51a+8b; 51a+9b; 51a+10b; 51a+11b; 51a+12b; 51a+13b; 51a+14b; 51a+15b; 51a+16b; 51a+17b; 51a+18b; 51a+19b; 51a+20b; 51a+21b; 51a+22b; 51a+23b; 51a+24b; 51a+25b; 51a+26b; 51a+27b; 51a+28b; 51a+29b; 51a+30b; 51a+31b; 51a+32b; 51a+33b; 51a+34b; 51a+35b; 51a+36b; 51a+37b; 51a+38b; 51a+39b; 51a+40b; 51a+41b; 51a+42b; 51a+43b; 51a+44b; 51a+45b; 51a+46b; 51a+47b; 51a+48b; 51a+49b; 51a+50b; 51a+51b; 51a+52b; 51a+53b; 51a+54b; 51a+55b; 51a+55b; 51a+57b; 51a+58b; 51a+59b; 51a+60b; 51a+61b; 51a+62b; 51a+63b; 51a+64b; 51a+65b; 51a+66b; 51a+67b; 51a+68b; 51a+69b; 51a+70b; 51a+71b; 51a+72b; 51a+73b; 51a+74b; 51a+75b; 51a+76b; 51a+77b; 51a+78b; 51a+79b; 51a+80b; 51a+81b; 51a+82b; 51a+83b; 51a+84b; 51a+85b; 51a+86b; 51a+87b; 51a+88b; 51a+89b; 51a+90b; 51a+91b; 51a+92b; 51a+93b; 51a+94b; 51a+95b; 51a+96b; 51a+97b; 52a+1b; 52a+2b; 52a+3b; 52a+4b; 52a+5b; 52a+6b; 52a+7b; 52a+8b; 52a+9b; 52a+10b; 52a+11b; 52a+12b; 52a+13b; 52a+14b; 52a+15b; 52a+16b; 52a+17b; 52a+18b; 52a+19b; 52a+20b; 52a+21b; 52a+22b; 52a+23b; 52a+24b; 52a+25b; 52a+26b; 52a+27b; 52a+28b; 52a+29b; 52a+30b; 52a+31b; 52a+32b; 52a+33b; 52a+34b; 52a+35b; 52a+36b; 52a+37b; 52a+38b; 52a+39b; 52a+40b; 52a+41b; 52a+42b; 52a+43b; 52a+44b; 52a+45b; 52a+46b; 52a+47b; 52a+48b; 52a+49b; 52a+50b; 52a+51b; 52a+52b; 52a+53b; 52a+54b; 52a+55b; 52a+55b; 52a+57b; 52a+58b; 52a+59b; 52a+60b; 52a+61b; 52a+62b; 52a+63b; 52a+64b; 52a+65b; 52a+66b; 52a+67b; 52a+68b; 52a+69b; 52a+70b; 52a+71b; 52a+72b; 52a+73b; 52a+74b; 52a+75b; 52a+76b; 52a+77b; 52a+78b; 52a+79b; 52a+80b; 52a+81b; 52a+82b; 52a+83b; 52a+84b; 52a+85b; 52a+86b; 52a+87b; 52a+88b; 52a+89b; 52a+90b; 52a+91b; 52a+92b; 52a+93b; 52a+94b; 52a+95b; 52a+96b; 52a+97b; 53a+1b; 53a+2b; 53a+3b; 53a+4b; 53a+5b; 53a+6b; 53a+7b; 53a+8b; 53a+9b; 53a+10b; 53a+11b; 53a+12b; 53a+13b; 53a+14b; 53a+15b; 53a+16b; 53a+17b; 53a+18b; 53a+19b; 53a+20b; 53a+21b; 53a+22b; 53a+23b; 53a+24b; 53a+25b; 53a+26b; 53a+27b; 53a+28b; 53a+29b; 53a+30b; 53a+31b; 53a+32b; 53a+33b; 53a+34b; 53a+35b; 53a+36b; 53a+37b; 53a+38b; 53a+39b; 53a+40b; 53a+41b; 53a+42b; 53a+43b; 53a+44b; 53a+45b; 53a+46b; 53a+47b; 53a+48b; 53a+49b; 53a+50b; 53a+51b; 53a+52b; 53a+53b; 53a+54b; 53a+55b; 53a+55b; 53a+57b; 53a+58b; 53a+59b; 53a+60b; 53a+61b; 53a+62b; 53a+63b; 53a+64b; 53a+65b; 53a+66b; 53a+67b; 53a+68b; 53a+69b; 53a+70b; 53a+71b; 53a+72b; 53a+73b; 53a+74b; 53a+75b; 53a+76b; 53a+77b; 53a+78b; 53a+79b; 53a+80b; 53a+81b; 53a+82b; 53a+83b; 53a+84b; 53a+85b; 53a+86b; 53a+87b; 53a+88b; 53a+89b; 53a+90b; 53a+91b; 53a+92b; 53a+93b; 53a+94b; 53a+95b; 53a+96b; 53a+97b; 54a+1b; 54a+2b; 54a+3b; 54a+4b; 54a+5b; 54a+6b; 54a+7b; 54a+8b; 54a+9b; 54a+10b; 54a+11b; 54a+12b; 54a+13b; 54a+14b; 54a+15b; 54a+16b; 54a+17b; 54a+18b; 54a+19b; 54a+20b; 54a+21b; 54a+22b; 54a+23b; 54a+24b; 54a+25b; 54a+26b; 54a+27b; 54a+28b; 54a+29b; 54a+30b; 54a+31b; 54a+32b; 54a+33b; 54a+34b; 54a+35b; 54a+36b; 54a+37b; 54a+38b; 54a+39b; 54a+40b; 54a+41b; 54a+42b; 54a+43b; 54a+44b; 54a+45b; 54a+46b; 54a+47b; 54a+48b; 54a+49b; 54a+50b; 54a+51b; 54a+52b; 54a+53b; 54a+54b; 54a+55b; 54a+55b; 54a+57b; 54a+58b; 54a+59b; 54a+60b; 54a+61b; 54a+62b; 54a+63b; 54a+64b; 54a+65b; 54a+66b; 54a+67b; 54a+68b; 54a+69b; 54a+70b; 54a+71b; 54a+72b; 54a+73b; 54a+74b; 54a+75b; 54a+76b; 54a+77b; 54a+78b; 54a+79b; 54a+80b; 54a+81b; 54a+82b; 54a+83b; 54a+84b; 54a+85b; 54a+86b; 54a+87b; 54a+88b; 54a+89b; 54a+90b; 54a+91b; 54a+92b; 54a+93b; 54a+94b; 54a+95b; 54a+96b; 54a+97b; 55a+1b; 55a+2b; 55a+3b; 55a+4b; 55a+5b; 55a+6b; 55a+7b; 55a+8b; 55a+9b; 55a+10b; 55a+11b; 55a+12b; 55a+13b; 55a+14b; 55a+15b; 55a+16b; 55a+17b; 55a+18b; 55a+19b; 55a+20b; 55a+21b; 55a+22b; 55a+23b; 55a+24b; 55a+25b; 55a+26b; 55a+27b; 55a+28b; 55a+29b; 55a+30b; 55a+31b; 55a+32b; 55a+33b; 55a+34b; 55a+35b; 55a+36b; 55a+37b; 55a+38b; 55a+39b; 55a+40b; 55a+41b; 55a+42b; 55a+43b; 55a+44b; 55a+45b; 55a+46b; 55a+47b; 55a+48b; 55a+49b; 55a+50b; 55a+51b; 55a+52b; 55a+53b; 55a+54b; 55a+55b; 55a+55b; 55a+57b; 55a+58b; 55a+59b; 55a+60b; 55a+61b; 55a+62b; 55a+63b; 55a+64b; 55a+65b; 55a+66b; 55a+67b; 55a+68b; 55a+69b; 55a+70b; 55a+71b; 55a+72b; 55a+73b; 55a+74b; 55a+75b; 55a+76b; 55a+77b; 55a+78b; 55a+79b; 55a+80b; 55a+81b; 55a+82b; 55a+83b; 55a+84b; 55a+85b; 55a+86b; 55a+87b; 55a+88b; 55a+89b; 55a+90b; 55a+91b; 55a+92b; 55a+93b; 55a+94b; 55a+95b; 55a+96b; 55a+97b; 56a+1b; 56a+2b; 56a+3b; 56a+4b; 56a+5b; 56a+6b; 56a+7b; 56a+8b; 56a+9b; 56a+10b; 56a+11b; 56a+12b; 56a+13b; 56a+14b; 56a+15b; 56a+16b; 56a+17b; 56a+18b; 56a+19b; 56a+20b; 56a+21b; 56a+22b; 56a+23b; 56a+24b; 56a+25b; 56a+26b; 56a+27b; 56a+28b; 56a+29b; 56a+30b; 56a+31b; 56a+32b; 56a+33b; 56a+34b; 56a+35b; 56a+36b; 56a+37b; 56a+38b; 56a+39b; 56a+40b; 56a+41b; 56a+42b; 56a+43b; 56a+44b; 56a+45b; 56a+46b; 56a+47b; 56a+48b; 56a+49b; 56a+50b; 56a+51b; 56a+52b; 56a+53b; 56a+54b; 56a+55b; 56a+55b; 56a+57b; 56a+58b; 56a+59b; 56a+60b; 56a+61b; 56a+62b; 56a+63b; 56a+64b; 56a+65b; 56a+66b; 56a+67b; 56a+68b; 56a+69b; 56a+70b; 56a+71b; 56a+72b; 56a+73b; 56a+74b; 56a+75b; 56a+76b; 56a+77b; 56a+78b; 56a+79b; 56a+80b; 56a+81b; 56a+82b; 56a+83b; 56a+84b; 56a+85b; 56a+86b; 56a+87b; 56a+88b; 56a+89b; 56a+90b; 56a+91b; 56a+92b; 56a+93b; 56a+94b; 56a+95b; 56a+96b; 56a+97b; 57a+1b; 57a+2b; 57a+3b; 57a+4b; 57a+5b; 57a+6b; 57a+7b; 57a+8b; 57a+9b; 57a+10b; 57a+11b; 57a+12b; 57a+13b; 57a+14b; 57a+15b; 57a+16b; 57a+17b; 57a+18b; 57a+19b; 57a+20b; 57a+21b; 57a+22b; 57a+23b; 57a+24b; 57a+25b; 57a+26b; 57a+27b; 57a+28b; 57a+29b; 57a+30b; 57a+31b; 57a+32b; 57a+33b; 57a+34b; 57a+35b; 57a+36b; 57a+37b; 57a+38b; 57a+39b; 57a+40b; 57a+41b; 57a+42b; 57a+43b; 57a+44b; 57a+45b; 57a+46b; 57a+47b; 57a+48b; 57a+49b; 57a+50b; 57a+51b; 57a+52b; 57a+53b; 57a+54b; 57a+55b; 57a+55b; 57a+57b; 57a+58b; 57a+59b; 57a+60b; 57a+61b; 57a+62b; 57a+63b; 57a+64b; 57a+65b; 57a+66b; 57a+67b; 57a+68b; 57a+69b; 57a+70b; 57a+71b; 57a+72b; 57a+73b; 57a+74b; 57a+75b; 57a+76b; 57a+77b; 57a+78b; 57a+79b; 57a+80b; 57a+81b; 57a+82b; 57a+83b; 57a+84b; 57a+85b; 57a+86b; 57a+87b; 57a+88b; 57a+89b; 57a+90b; 57a+91b; 57a+92b; 57a+93b; 57a+94b; 57a+95b; 57a+96b; 57a+97b; 58a+1b; 58a+2b; 58a+3b; 58a+4b; 58a+5b; 58a+6b; 58a+7b; 58a+8b; 58a+9b; 58a+10b; 58a+11b; 58a+12b; 58a+13b; 58a+14b; 58a+15b; 58a+16b; 58a+17b; 58a+18b; 58a+19b; 58a+20b; 58a+21b; 58a+22b; 58a+23b; 58a+24b; 58a+25b; 58a+26b; 58a+27b; 58a+28b; 58a+29b; 58a+30b; 58a+31b; 58a+32b; 58a+33b; 58a+34b; 58a+35b; 58a+36b; 58a+37b; 58a+38b; 58a+39b; 58a+40b; 58a+41b; 58a+42b; 58a+43b; 58a+44b; 58a+45b; 58a+46b; 58a+47b; 58a+48b; 58a+49b; 58a+50b; 58a+51b; 58a+52b; 58a+53b; 58a+54b; 58a+55b; 58a+55b; 58a+57b; 58a+58b; 58a+59b; 58a+60b; 58a+61b; 58a+62b; 58a+63b; 58a+64b; 58a+65b; 58a+66b; 58a+67b; 58a+68b; 58a+69b; 58a+70b; 58a+71b; 58a+72b; 58a+73b; 58a+74b; 58a+75b; 58a+76b; 58a+77b; 58a+78b; 58a+79b; 58a+80b; 58a+81b; 58a+82b; 58a+83b; 58a+84b; 58a+85b; 58a+86b; 58a+87b; 58a+88b; 58a+89b; 58a+90b; 58a+91b; 58a+92b; 58a+93b; 58a+94b; 58a+95b; 58a+96b; 58a+97b; 59a+1b; 59a+2b; 59a+3b; 59a+4b; 59a+5b; 59a+6b; 59a+7b; 59a+8b; 59a+9b; 59a+10b; 59a+11b; 59a+12b; 59a+13b; 59a+14b; 59a+15b; 59a+16b; 59a+17b; 59a+18b; 59a+19b; 59a+20b; 59a+21b; 59a+22b; 59a+23b; 59a+24b; 59a+25b; 59a+26b; 59a+27b; 59a+28b; 59a+29b; 59a+30b; 59a+31b; 59a+32b; 59a+33b; 59a+34b; 59a+35b; 59a+36b; 59a+37b; 59a+38b; 59a+39b; 59a+40b; 59a+41b; 59a+42b; 59a+43b; 59a+44b; 59a+45b; 59a+46b; 59a+47b; 59a+48b; 59a+49b; 59a+50b; 59a+51b; 59a+52b; 59a+53b; 59a+54b; 59a+55b; 59a+55b; 59a+57b; 59a+58b; 59a+59b; 59a+60b; 59a+61b; 59a+62b; 59a+63b; 59a+64b; 59a+65b; 59a+66b; 59a+67b; 59a+68b; 59a+69b; 59a+70b; 59a+71b; 59a+72b; 59a+73b; 59a+74b; 59a+75b; 59a+76b; 59a+77b; 59a+78b; 59a+79b; 59a+80b; 59a+81b; 59a+82b; 59a+83b; 59a+84b; 59a+85b; 59a+86b; 59a+87b; 59a+88b; 59a+89b; 59a+90b; 59a+91b; 59a+92b; 59a+93b; 59a+94b; 59a+95b; 59a+96b; 59a+97b; 60a+1b; 60a+2b; 60a+3b; 60a+4b; 60a+5b; 60a+6b; 60a+7b; 60a+8b; 60a+9b; 60a+10b; 60a+11b; 60a+12b; 60a+13b; 60a+14b; 60a+15b; 60a+16b; 60a+17b; 60a+18b; 60a+19b; 60a+20b; 60a+21b; 60a+22b; 60a+23b; 60a+24b; 60a+25b; 60a+26b; 60a+27b; 60a+28b; 60a+29b; 60a+30b; 60a+31b; 60a+32b; 60a+33b; 60a+34b; 60a+35b; 60a+36b; 60a+37b; 60a+38b; 60a+39b; 60a+40b; 60a+41b; 60a+42b; 60a+43b; 60a+44b; 60a+45b; 60a+46b; 60a+47b; 60a+48b; 60a+49b; 60a+50b; 60a+51b; 60a+52b; 60a+53b; 60a+54b; 60a+55b; 60a+55b; 60a+57b; 60a+58b; 60a+59b; 60a+60b; 60a+61b; 60a+62b; 60a+63b; 60a+64b; 60a+65b; 60a+66b; 60a+67b; 60a+68b; 60a+69b; 60a+70b; 60a+71b; 60a+72b; 60a+73b; 60a+74b; 60a+75b; 60a+76b; 60a+77b; 60a+78b; 60a+79b; 60a+80b; 60a+81b; 60a+82b; 60a+83b; 60a+84b; 60a+85b; 60a+86b; 60a+87b; 60a+88b; 60a+89b; 60a+90b; 60a+91b; 60a+92b; 60a+93b; 60a+94b; 60a+95b; 60a+96b; 60a+97b; etc.

Within certain embodiments of the invention, the therapeutic composition can also comprise radio-opaque, echogenic materials and magnetic resonance imaging (MRI) responsive materials (i.e., MRI contrast agents) to aid in visualization of the composition under ultrasound, fluoroscopy and/or MRI. For example, a composition may be echogenic or radiopaque (e.g., made with echogenic or radiopaque with materials such as powdered tantalum, tungsten, barium carbonate, bismuth oxide, barium sulfate, metrazimide, iopamidol, iohexol, iopromide, iobitridol, iomeprol, iopentol, ioversol, ioxilan, iodixanol, iotrolan, acetrizoic acid derivatives, diatrizoic acid derivatives, iothalamic acid derivatives, ioxithalamic acid derivatives, metrizoic acid derivatives, iodamide, lypophylic agents, iodipamide and ioglycamic acid or, by the addition of microspheres or bubbles which present an acoustic interface). For visualization under MRI, contrast agents (e.g., gadolinium (III) chelates or iron oxide compounds) may be incorporated into the composition.

The compositions may, alternatively, or in addition, be visualized under visible light, using fluorescence, or by other spectroscopic means. Visualization agents that can be included for this purpose include dyes, pigments, and other colored agents. In one aspect, the composition may further include a colorant to improve visualization of the composition in vivo and/or ex vivo. Frequently, compositions can be difficult to visualize upon delivery into a host, especially at the margins of an implant or tissue. A coloring agent can be incorporated into a composition to reduce or eliminate the incidence or severity of this problem. The coloring agent provides a unique color, increased contrast, or unique fluorescence characteristics to the composition. In one aspect, a composition is provided that includes a colorant such that it is readily visible (under visible light or using a fluorescence technique) and easily differentiated from its implant site. In another aspect, a colorant can be included in a liquid or semi-solid composition. For example, a single component of a two component mixture may be colored, such that when combined ex-vivo or in-vivo, the mixture is sufficiently colored.

The coloring agent may be, for example, an endogenous compound (e.g., an amino acid or vitamin) or a nutrient or food material and may be a hydrophobic or a hydrophilic compound. Preferably, the colorant has a very low or no toxicity at the concentration used. Also preferred are colorants that are safe and normally enter the body through absorption such as β-carotene. Representative examples of colored nutrients (under visible light) include fat soluble vitamins such as Vitamin A (yellow); water soluble vitamins such as Vitamin B12 (pink-red) and folic acid (yellow-orange); carotenoids such as β-carotene (yellow-purple) and lycopene (red). Other examples of coloring agents include natural product (berry and fruit) extracts such as anthrocyanin (purple) and saffron extract (dark red). The coloring agent may be a fluorescent or phosphorescent compound such as α-tocopherolquinol (a Vitamin E derivative) or L-tryptophan.

In one aspect, the compositions of the present invention include one or more coloring agents, also referred to as dyestuffs, which will be present in an effective amount to impart observable coloration to the composition, e.g., the gel. Examples of coloring agents include dyes suitable for food such as those known as F. D. & C. dyes and natural coloring agents such as grape skin extract, beet red powder, beta carotene, annato, carmine, turmeric, paprika, and so forth. Derivatives, analogues, and isomers of any of the above colored compound also may be used. The method for incorporating a colorant into an implant or therapeutic composition may be varied depending on the properties of and the desired location for the colorant. For example, a hydrophobic colorant may be selected for hydrophobic matrices. The colorant may be incorporated into a carrier matrix, such as micelles. Further, the pH of the environment may be controlled to further control the color and intensity.

In one aspect, the compositions of the present invention include one or more preservatives or bacteriostatic agents present in an effective amount to preserve the composition and/or inhibit bacterial growth in the composition, for example, bismuth tribromophenate, methyl hydroxybenzoate, bacitracin, ethyl hydroxybenzoate, propyl hydroxybenzoate, erythromycin, chlorocresol, benzalkonium chlorides, and the like. Examples of the preservative include paraoxybenzoic acid esters, chlorobutanol, benzylalcohol, phenethyl alcohol, dehydroacetic acid, sorbic acid, etc. In one aspect, the compositions of the present invention include one or more bactericidal (also known as bacteriacidal) agents.

In one aspect, the compositions of the present invention include one or more antioxidants, present in an effective amount. Examples of the antioxidant include sulfites, alpha-tocopherol, beta-carotene and ascorbic acid.

Further, therapeutic compositions of the present invention should preferably be have a stable shelf-life of at least several months and capable of being produced and maintained under sterile conditions. The composition may be sterile either by preparing them under aseptic environment and/or they may be terminally sterilized using methods known in the art. A combination of both of these methods may also be used to prepare the composition in the sterile form. Sterilization may also occur by terminally using gamma radiation or electron beam sterilization methods.

In one aspect, the compounds and compositions of the present invention are sterile. Many pharmaceuticals are manufactured to be sterile and this criterion is defined by the USP XXII <1211>. The term “USP” refers to U.S. Pharmacopeia (see www.usp.org, Rockville, Md.). Sterilization in this embodiment may be accomplished by a number of means accepted in the industry and listed in the USP XXII <1211>, including gas sterilization, ionizing radiation or, when appropriate, filtration. Sterilization may be maintained by what is termed asceptic processing, defined also in USP XXII <1211>. Acceptable gases used for gas sterilization include ethylene oxide. Acceptable radiation types used for ionizing radiation methods include gamma, for instance from a cobalt 60 source and electron beam. A typical dose of gamma radiation is 2.5 MRad. Filtration may be accomplished using a filter with suitable pore size, for example 0.22 μm and of a suitable material, for instance polytetrafluoroethylene (e.g., TEFLON from E. I. DuPont De Nemours and Company, Wilmington, Del.).

In another aspect, the compositions of the present invention are contained in a container that allows them to be used for their intended purpose, i.e., as a pharmaceutical composition. Properties of the container that are important are a volume of empty space to allow for the addition of a constitution medium, such as water or other aqueous medium, e.g., saline, acceptable light transmission characteristics in order to prevent light energy from damaging the composition in the container (refer to USP XXII <661>), an acceptable limit of extractables within the container material (refer to USP XXII), an acceptable barrier capacity for moisture (refer to USP XXII <671>) or oxygen. In the case of oxygen penetration, this may be controlled by including in the container, a positive pressure of an inert gas, such as high purity nitrogen, or a noble gas, such as argon.

Typical materials used to make containers for pharmaceuticals include USP Type I through III and Type NP glass (refer to USP XXII <661>), polyethylene, TEFLON, silicone, and gray-butyl rubber. For parenterals, USP Types I to III glass and polyethylene are preferred.

E. Methods for Utilizing Compositions

The compositions of the present invention can be used in a variety of different applications. For example, the compositions may be used for (a) preventing tissue adhesions; (b) treating or preventing inflammatory arthritis; (c) prevention of cartilage loss; (d) treating or preventing hypertrophic scars/keloids; (e) treating or preventing vascular disease; and (f) coating medical implants and devices. A more detailed description of several specific applications is given below.

Adhesion Prevention

The present invention provides compositions for use in the prevention of adhesions (e.g., surgical adhesions). The polymeric compositions may include one or more therapeutically active agents (e.g., anti-scarring agents), which provide pharmacological alteration of cellular and/or non-cellular processes involved in the development and/or progression of surgical adhesions. Therapeutically active agents are described that can reduce surgical adhesions by inhibiting the formation of fibrous or scar tissue. In another aspect, the present invention provides surgical adhesion barriers that include an anti-scarring agent or a composition that includes an anti-scarring agent.

Surgical adhesions are abnormal, fibrous bands of scar tissue that can form inside the body as a result of the healing process that follows any open or minimally invasive surgical procedure including abdominal, gynecologic, cardiothoracic, spinal, plastic, vascular, ENT, ophthalmologic, urologic, neuro, or orthopedic surgery. Surgical adhesions are typically connective tissue structures that form between adjacent injured areas within the body. Briefly, localized areas of injury trigger an inflammatory and healing response that culminates in healing and scar tissue formation. If scarring results in the formation of fibrous tissue bands or adherence of adjacent anatomical structures (that should be separate), surgical adhesion formation is said to have occurred. Adhesions can range from flimsy, easily separable structures to dense, tenacious fibrous structures that can only be separated by surgical dissection. While many adhesions are benign, some can cause significant clinical problems and are a leading cause of repeat surgical intervention. Surgery to breakdown adhesions (adhesiolysis) often results in failure and recurrence because the surgical trauma involved in breaking down the adhesion triggers the entire process to repeat itself. Surgical breakdown of adhesions is a significant clinical problem and it is estimated that there were 473,000 adhesiolysis procedures in the US in 2002. According to the Diagnosis-Related Groups (DRGs), the total hospital charges for these procedures is likely to be at least US $10 billion annually.

Since all interventions involve a certain degree of trauma to the operative tissues, virtually any procedure (no matter how well executed) has the potential to result in the formation of clinically significant adhesion formation. Adhesions can be triggered by surgical trauma such as cutting, manipulation, retraction or suturing, as well as from inflammation, infection (e.g., fungal or mycobacterium), bleeding or the presence of a foreign body. Surgical trauma may also result from tissue drying, ischemia, or thermal injury. Due to the diverse etiology of surgical adhesions, the potential for formation exists regardless of whether the surgery is done in a so-called minimally invasive fashion (e.g., catheter-based therapies, laparoscopy) or in a standard open technique involving one or more relatively large incisions. Although a potential complication of any surgical intervention, surgical adhesions are particularly problematic in GI surgery (causing bowel obstruction), gynecological surgery (causing pain and/or infertility), tendon repairs (causing shortening and flexion deformities), joint capsule procedures (causing capsular contractures), and nerve and muscle repair procedures (causing diminished or lost function).

Surgical adhesions may cause various, often serious and unpredictable clinical complications; some of which manifest themselves only years after the original procedure was completed. Complications from surgical adhesions are a major cause of failed surgical therapy and are the leading cause of bowel obstruction and infertility. Other adhesion-related complications include chronic back or pelvic pain, intestinal obstruction, urethral obstruction and voiding dysfunction. Relieving the post-surgical complications caused by adhesions generally requires another surgery. However, the subsequent surgery is further complicated by adhesions formed as a result of the previous surgery. In addition, the second surgery is likely to result in further adhesions and a continuing cycle of additional surgical complications.

The placement of medical devices and implants also increases the risk that surgical adhesions will occur. In addition to the above mechanisms, an implanted device can trigger a “foreign body” response where the immune system recognizes the implant as foreign and triggers an inflammatory reaction that ultimately leads to scar tissue formation. A specific form of foreign body reaction in response to medical device placement is complete enclosure (“walling off”) of the implant in a capsule of scar tissue (encapsulation). Fibrous encapsulation of implanted devices and implants can complicate any procedure, but breast augmentation and reconstruction surgery, joint replacement surgery, hernia repair surgery, artificial vascular graft surgery, stent placement, and neurosurgery are particularly prone to this complication. In each case, the implant becomes encapsulated by a fibrous connective tissue capsule which compromises or impairs the function of the surgical implant (e.g., breast implant, artificial joint, surgical mesh, vascular graft, stent or dural patch).

Adhesions generally begin to form within the first several days after surgery. Generally, adhesion formation is an inflammatory reaction in which factors are released, increasing vascular permeability and resulting in fibrinogen influx and fibrin deposition. This deposition forms a matrix that bridges the abutting tissues. Fibroblasts accumulate, attach to the matrix, deposit collagen and induce angiogenesis. If this cascade of events can be prevented within 4 to 5 days following surgery, then adhesion formation may be inhibited.

Various modes of adhesion prevention have been examined, including (1) prevention of fibrin deposition, (2) reduction of local tissue inflammation and (3) removal of fibrin deposits. Fibrin deposition is prevented through the use of physical barriers that are either mechanical or comprised of viscous solutions. Barriers have the added advantage of physically preventing adjacent tissues from contacting each other and thereby reducing the probability that they will scar together. Although many investigators and commercial products utilize adhesion prevention barriers, a number of technical difficulties exist and significant failure rates have been reported. Inflammation is reduced by the administration of drugs such as corticosteroids and non-steroidal anti-inflammatory drugs. However, the results from the use of these drugs in animal models have not been encouraging due to the extent of the inflammatory response and dose restriction due to systemic side effects. Finally, the removal of fibrin deposits has been investigated using proteolytic and fibrinolytic enzymes. A potential complication to the clinical use of these enzymes is the possibility for post-surgical excessive bleeding (surgical hemostasis is critical for procedural success).

Numerous polymeric compositions for use in the prevention of surgical adhesions (e.g., surgical adhesion barriers) may be used in the practice of the invention, either alone, or in combination with one or more anti-scarring agents. It should be noted that certain polymeric compositions can themselves help prevent the formation of fibrous tissue at a surgical site. In certain embodiments, the polymer composition can form a barrier between the tissue surfaces or organs.

For example, the surgical adhesion barrier may be coated onto tissue surfaces and may be composed of an aqueous solution of a hydrophilic, polymeric material (e.g., polypeptides or polysaccharide) having greater than 50,000 molecular weight and a concentration range of 0.01% to 15% by weight. See e.g., U.S. Pat. No. 6,464,970. The surgical adhesion barrier may be a crosslinkable system with at least three reactive compounds each having a polymeric molecular core with at least one functional group. See e.g., U.S. Pat. No. 6,458,889. The surgical adhesions barrier may be composed of a non-gelling polyoxyalkylene composition with or without a therapeutic agent. See e.g., U.S. Pat. No. 6,436,425. The surgical adhesions barrier may be composed of an anionic polymer having an acid sulfate and sulfur content greater than 5% which acts to inhibit monocyte or macrophage invasion. See e.g., U.S. Pat. No. 6,417,173. The surgical adhesions barrier may be an aqueous composition including a surfactant, pentoxifylline and a polyoxyalkylene polyether. See e.g., U.S. Pat. No. 6,399,624. The surgical adhesions barrier may be composed by crosslinking two synthetic polymers, one having nucleophilic groups and the other having electrophilic groups, such that they form a matrix that may be used to incorporate a biologically active compound. See e.g., U.S. Pat. Nos. 6,323,278; 6,166,130; 6,051,648 and 5,874,500. The surgical adhesion barrier may be composed of hyaluronic acid compositions such as those described in U.S. Pat. Nos. 6,723,709; 6,531,147; and 6,464,970. The surgical adhesions barrier may be a polymeric tissue coating which is formed by applying a polymerization initiator to the tissue and then covering it with a water-soluble macromer that is polymerizable using free radical initiators under the influence of UV light. See e.g., U.S. Pat. Nos. 6,177,095 and 6,083,524. The surgical adhesions barrier may be composed of fluent prepolymeric material that is emitted to the tissue surface and then exposed to activating energy in situ to initiate conversion of the applied material to non-fluent polymeric form. See e.g., U.S. Pat. Nos. 6,004,547 and 5,612,050. The surgical adhesions barrier may be a hydrogel-forming, self-solvating, absorbable polyester copolymers capable of selective, segmental association into compliant hydrogels mass upon contact with an aqueous environment. See e.g., U.S. Pat. No. 5,612,052. The surgical adhesions barrier may be an anionic polymer effective to inhibit cell invasion or fibrosis (e.g., dermatan sulfate, dextran sulfate, pentosan polysulfate, or alginate), and a pharmaceutically effective carrier, in which the carrier may be semi-solid. See e.g., U.S. Pat. Nos. 6,756,362; 6,127,348 and 5,994,325. The surgical adhesions barrier may be an acidified hydrogel comprising a carboxypolysaccharide and a polyether having a pH in the range of about 2.0 to about 6.0. See e.g., U.S. Pat. No. 6,017,301. The surgical adhesions barrier may be composed of dextran sulfate having a molecular weight about 40,000 to 500,000 Daltons which is used to inhibit neurite outgrowth. See e.g., U.S. Pat. No. 5,705,178. The surgical adhesions barrier may be a fragmented biocompatible hydrogel which is at least partially hydrated and is substantially free from an aqueous phase, wherein said hydrogel comprises gelatin and will absorb water when delivered to a moist tissue target site. See e.g., U.S. Pat. No. 6,066,325. The surgical adhesions barrier may be a water-soluble, degradable macromer that is composed of at least two-crosslinkable substituents that may crosslink to other macromers at a localized site when under the influence of a polymerization initiator. See e.g., U.S. Pat. No. 6,465,001. The surgical adhesions barrier may be a biocompatible adhesive composition comprising at least one alkyl ester cyanoacrylate monomer and a polymerization initiator or accelerator. See e.g., U.S. Pat. No. 6,620,846.

In one embodiment, the polymers that can form a covalent bond with the tissue to which it is applied may be used. Polymers containing and/or terminated with electrophilic groups such as succinimidyl, aldehyde, epoxide, isocyanate, vinyl, vinyl sulfone, maleimide, —S—S—(C₅H₄N) or activated esters, such as are used in peptide synthesis may be used as the reagents. For example, a 4 armed NHS-derivatized polyethylene glycol (e.g., pentaerythritol poly(ethylene glycol)ether tetra-succinimidyl glutarate) may be applied to the tissue in the solid form or in a solution form. In this embodiment, the 4 armed NHS-derivatized polyethylene glycol is dissolved in an acidic solution (pH about 2-3) and is then co-applied to the tissue using a basic buffer (pH>about 8). The fibrosis-inhibiting agent(s) may be incorporated directly into either the 4 armed NHS-derivatized polyethylene glycol, the acidic solution or the basic buffer. In another embodiment, the fibrosis-inhibiting agent may be incorporated into a secondary carrier that may then be incorporated into the 4 armed NHS-derivatized polyethylene glycol, the acidic solution and/or the basic buffer. Secondary carriers may include microparticles and/or microspheres which are made from degradable polymers. Degradable polymers may include polyesters, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X—Y, Y—X—Y, R—(Y—X)ₙ, R—(X—Y), and X—Y—X (where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator).

In another embodiment, the tissue reactive polymer may be applied initially and then the fibrosis-inhibiting agent may then be applied to the coated tissue. The fibrosis-inhibiting agent may be applied directly to the tissue or it may be incorporated into a secondary carrier. Secondary carriers may include microspheres (as described above), microparticles (as described above), gels (e.g., hyaluronic acid, carboxymethyl cellulose, dextran, poly(ethylene oxide)—poly(propylene oxide) block copolymers as well as blends, association complexes and crosslinked compositions thereof) and films (degradable polyesters, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X—Y, Y—X—Y, R—(Y—X)ₙ, R—(X—Y), and X—Y—X (where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator, hyaluronic acid, carboxymethyl cellulose, dextran, poly(ethylene oxide)—poly(propylene oxide) block copolymers as well as blends, association complexes and crosslinked compositions thereof.

A preferred polymeric matrix which can be used to help prevent the formation of fibrous tissue, either alone or in combination with a fibrosis inhibiting agent/composition, is formed from reactants comprising either one or both of pentaerythritol poly(ethylene glycol)ether tetra-sulfhydryl] (4-armed thiol PEG, which includes structures having a linking group(s) between a sulfhydryl group(s) and the terminus of the polyethylene glycol backbone) and pentaerythritol poly(ethylene glycol)ether tetra-succinimidyl glutarate] (4-armed NHS PEG, which again includes structures having a linking group(s) between a NHS group(s) and the terminus of the polyethylene glycol backbone) as reactive reagents. Another preferred composition comprises either one or both of pentaerythritol poly(ethylene glycol)ether tetra-amino] (4-armed amino PEG, which includes structures having a linking group(s) between an amino group(s) and the terminus of the polyethylene glycol backbone) and pentaerythritol poly(ethylene glycol)ether tetra-succinimidyl glutarate] (4-armed NHS PEG, which again includes structures having a linking group(s) between a NHS group(s) and the terminus of the polyethylene glycol backbone) as reactive reagents. Chemical structures for these reactants are shown in, e.g., U.S. Pat. No. 5,874,500. Optionally, collagen or a collagen derivative (e.g., methylated collagen) is added to the poly(ethylene glycol)-containing reactant(s) to form a preferred crosslinked matrix that can serve as a polymeric carrier for a therapeutic agent or a stand-alone composition to help prevent the formation of fibrous tissue.

Surgical adhesion barriers, which may be combined with one or more anti-scarring agents according to the present invention, also include commercially available products. Examples of surgical adhesion barrier compositions into which a fibrosis agent can be incorporated include: (a) sprayable collagen-containing formulations such as COSTASIS or CT3 (Angiotech Pharmaceuticals, Inc., Canada); (b) sprayable PEG-containing formulations such as COSEAL or ADHIBIT (Angiotech Pharmaceuticals, Inc.), SPRAYGEL or DURASEAL (both from Confluent Surgical, Inc., Boston, Mass.) or FOCALSEAL (Genzyme Corporation, Cambridge, Mass.); (c) hyaluronic acid-containing formulations such as RESTYLANE or PERLANE (both from Q-Med AB, Sweden), HYLAFORM (Inamed Corporation, Santa Barbara, Calif.), SYNVISC (Biomatrix, Inc., Ridgefield, N.J.), SEPRAFILM or SEPRACOAT (both from Genzyme Corporation), (d) fibrinogen-containing formulations such as FLOSEAL or TISSEAL (both from Baxter Healthcare Corporation, Fremont, Calif.); (e) polymeric gels such as REPEL (Life Medical Sciences, Inc., Princeton, N.J.) or FLOWGEL (Baxter Healthcare Corporation, Deerfield, Ill.), (f) surgical adhesives containing cyanoacrylates such as DERMABOND (Johnson & Johnson, Inc., New Brunswick, N.J.), INDERMIL (U.S. Surgical Company, Norwalk, Conn.), GLUSTITCH (Blacklock Medical Products Inc., Canada), TISSUMEND (Veterinary Products Laboratories, Phoenix, Ariz.), VETBOND (3M Company, St. Paul, Minn.), HISTOACRYL BLUE (Davis & Geck, St. Louis, Mo.) and ORABASE SOOTHE-N-SEAL LIQUID PROTECTANT (Colgate-Palmolive Company, New York, N.Y.); (g) dextran sulfate gels such as the ADCON range of products (available from Wright Medical Technology, Inc. Arlington, Tenn.), (h) lipid based compositions such as ADSURF (Britannia Pharmaceuticals Ltd., United Kingdom) and (j) film compositions such as INTERCEED (Ethicon, Inc., Somerville, N.J.) and HYDROSORB (MacroPore Biosurgery, Inc., San Diego, Calif./Medtronic Sofamor Danek, Memphis, Tenn.).

For greater clarity, several specific applications and treatments will be described in greater detail including:

i) Adhesion Prevention in Spinal and Neurosurgical Procedures

Back pain is the number one cause of healthcare expenditures in the United States and accounts for over $50 billion in costs annually ($100 billion worldwide). Over 12 million people in the U.S. have some form of degenerative disc disease (DDD) and 10% of them (1.2 million) will require surgery to correct their problem.

In healthy individuals, the vertebral column is composed of vertebral bone plates separated by intervertebral discs that form strong joints and absorb spinal compression during movement. The intervertebral disc is comprised of an inner gel-like substance called the nucleus pulposus which is surrounded by a tough fibrocartilagenous capsule called the annulus fibrosis. The nucleus pulposus is composed of a loose framework of collagen fibrils and connective tissue cells (resembling fibroblasts and chondrocytes) embedded in a gelatinous matrix of glycosaminoglycans and water. The annulus fibrosus is composed of numerous concentric rings of fibrocartilage that anchor into the vertebral bodies. The most common cause of DDD occurs when tears in the annulus fibrosis create an area of localized weakness that allow bulging, herniation or sequestration of the nucleus pulposis and annulus fibrosis into the spinal canal and/or spinal foramena. The bulging or herniated disc often compresses nerve tissue such as spinal cord fibers or spinal cord nerve root fibers. Pressure on the spinal cord or nerve roots from the damaged intervertebral disc results in neuronal dysfunction (numbness, weakness, tingling), crippling pain, bowel or bladder disturbances and can frequently cause long-term disability. Although many cases of DDD will spontaneously resolve, a significant number of patients will require surgical intervention in the form of minimally invasive procedures, microdiscectomy, major surgical resection of the disc, spinal fusion (fusion of adjacent vertebral bone plates using various techniques and devices), and/or implantation of an artificial disc. The present invention provides for the application of an anti-adhesion or anti-fibrosis agent in the surgical management of DDD.

Spinal disc removal is mandatory and urgent in cauda equine syndrome when there is a significant neurological deficit; particularly bowel or bladder dysfunction. It is also performed electively to relieve pain and eliminate lesser neurological symptoms. The spinal nerve roots exit the spinal canal through bony spinal foramena (a bony opening between the vertebra above and the vertebra below) that is a common site of nerve entrapment. To gain access to the spinal foramen during back surgeries, vertebral bone tissue is often resected; a process known as laminectomy.

In open surgical resection of a ruptured lumbar disc or entrapped spinal nerve root (laminectomy) the patient is placed in a modified kneeling position under general anesthesia. An incision is made in the posterior midline and the tissue is dissected away to expose the appropriate interspace; the ligamentum flavum is dissected and in some cases portions of the bony lamina are removed to allow adequate visualization. The nerve root is carefully retracted away to expose the herniated fragment and the defect in the annulus. Typically, the cavity of the disc is entered from the tear in the annulus and the loose fragments of the nucleus pulposus are removed with pituitary forceps. Any additional fragments of disc sequestered inside or outside of the disc space are also carefully removed and the disc space is forcefully irrigated to remove to remove any residual fragments. If tears are present in the dura, the dura is closed with sutures that are often augmented with fibrin glue. The tissue is then closed with absorbable sutures.

Microlumbar disc excision (microdiscectomy) can be performed as an outpatient procedure and has largely replaced laminectomy as the intervention of choice for herniated discs or root entrapment. A one inch incision is made from the spinous process above the disc affected to the spinous process below. Using an operating microscope, the tissue is dissected down to the ligamentum flavum and bone is removed from the lamina until the nerve root can be clearly identified. The nerve root is carefully retracted and the tears in the annulus are visualized under magnification. Microdisc forceps are used to remove disc fragments through the annular tear and any sequestered disc fragments are also removed. As with laminectomy, the disc space is irrigated to remove any disc fragments, any dural tears are repaired and the tissue is closed with absorbable sutures. It should be noted that anterior (abdominal) approaches can also be used for both open and endoscopic lumbar disc excision. Cervical and thoracic disc excisions are similar to lumbar procedures and can also be performed from a posterior approach (with laminectomy) or as an anterior discectomy with fusion.

Back surgeries, such as laminectomies, discectomies and microdiscectomies, often leave the spinal dura exposed and unprotected. As a result, scar tissue frequently forms between the dura and the surrounding tissue. This scar is formed from the damaged erector spinae muscles that overlay the laminectomy site. The result is adhesion development between the muscle tissue and the fragile dura, thereby, reducing mobility of the spine and the nerve roots that exit from it, leading to pain, persistent neurological symptoms and slow post-operative recovery. Similarly, adhesions that occur in the epidural and dural tissue cause complications in spinal injury (e.g., compression and crush injuries) cases. In addition, scar and adhesion formation within the dura and around nerve roots has been implicated in rendering subsequent (revision and repeat) spine operations technically more difficult to perform.

To circumvent adhesion development, a scar-reducing barrier may be inserted between the dural sleeve and the paravertebral musculature post-laminectomy. Alternatively (or in addition to this), the adhesion barrier, either alone or containing a fibrosis-inhibiting agent, can be coated on (or infiltrated into the tissues around) the spinal nerve as it exits the spinal canal and traverses the space between the bony vertebra (i.e., the laminectomy site). This reduces cellular and vascular invasion into the epidural space from the overlying muscle and exposed cancellous bone and thus, reduces the complications associated with scarring of the canal housing, spinal chord and/or nerve roots. In microdiscectomy procedures it is important that the barrier be deliverable as a spray, gel or fluid material that can be administered via the delivery port of an endoscope. Once again, the adhesion barrier, either alone or containing a fibrosis-inhibiting agent, can be sprayed onto the spinal nerve (or infiltrated into the tissues around it) as it exits the spinal canal and traverses the space between the bony vertebra (i.e., the laminectomy site). The present invention discloses barrier compositions, used either alone or combined with a fibrosis-inhibiting agent, that can be delivered during surgical disc resection and microdiscectomy either directly, using specialized delivery catheters, via an endoscope, or through a needle or other applicator. When dural defects are present, the fibrosis-inhibiting agent will assist in the healing of the dura and prevent complications such as blockage of CSF flow.

In another aspect, adhesion formation may be associated with a neurosurgical (brain) procedure. Neurosurgical procedures are fraught with potentially severe post-operative complications that are often attributed to surgical trauma and unwanted fibrosis or gliosis (gliosis is scar tissue formation in the brain as a result of glial cell activity). Increased intracranial bleeding, infection, cerebrospinal fluid leakage and pain are but some complications resulting from adhesions following neurosurgery. For example, if scar tissue interrupts the normal circulation of cerebrospinal fluid (CSF) following brain or spinal surgery, the fluid can accumulate and exert pressure on surrounding tissues (causing increased intracranial pressure) leading to severe complications (such as uncal herneation, brain damage and/or death). Here the adhesion barrier alone, or combined with a fibrosis-inhibiting agent, can be used to prevent excessive dural scarring and adhesion formation in a variety of neurosurgical procedures.

There are numerous compositions that may be used alone or loaded with a therapeutic agent (e.g., a fibrosis-inhibiting agent or an anti-infective agent), applied to a spinal or neurosurgical site (or to an implant surface placed in the spine—such as an artificial disc, rods, screws, spinal cages, drug-delivery pumps, neurostimulation devices; or to an implant placed in the brain—such as drains, shunts, drug-delivery pumps, neurostimulation devices) for the prevention of surgical adhesions in neurosurgical procedures. It should be noted that certain polymeric compositions can themselves help prevent the formation of fibrous tissue at a spinal or neurosurgical site. These compositions are particularly useful for the practice of this embodiment, either alone, or in combination with a fibrosis-inhibiting composition.

Various polymeric compositions can be infiltrated into the spinal or neurosurgical site (e.g., onto tissue at the surgical site or in the vicinity of the implant-tissue interface) with or without an additional therapeutic agent for the prevention of surgical adhesions.

In one embodiment, the polymers that can form a covalent bond with the tissue to which it is applied may be used. Polymers containing and/or terminated with electrophilic groups such as succinimidyl, aldehyde, epoxide, isocyanate, vinyl, vinyl sulfone, maleimide, —S—S—(C₅H₄N) or activated esters, such as are used in peptide synthesis may be used as the reagents. For example, a 4 armed NHS-derivatized polyethylene glycol (e.g., pentaerythritol poly(ethylene glycol)ether tetra-succinimidyl glutarate) may be applied to the tissue in the solid form or in a solution form. In this embodiment, the 4 armed NHS-derivatized polyethylene glycol is dissolved in an acidic solution (pH about 2-3) and is then co-applied to the tissue using a basic buffer (pH>about 8). The antifibrosisfibrosis-inhibiting agent(s) may be incorporated directly into either the 4 armed NHS-derivatized polyethylene glycol, the acidic solution or the basic buffer.

In another embodiment, the fibrosis-inhibiting agent may be incorporated into a secondary carrier that may then be incorporated into the 4 armed NHS-derivatized polyethylene glycol, the acidic solution and/or the basic buffer. The secondary carriers may include microparticles and/or microspheres which are made from degradable polymers. The degradable polymers may include polyesters, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X—Y, Y—X—Y, R—(Y—X)ₙ, R—(X—Y)ₙ and X—Y—X (where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator).

In another embodiment, the tissue reactive polymer may be applied initially and then the fibrosis-inhibiting agent may then be applied to the coated tissue. The fibrosis-inhibiting agent may be applied directly to the tissue or it may be incorporated into a secondary carrier. The secondary carriers may include microspheres (as described above), microparticles (as described above), gels (e.g., hyaluronic acid, carboxymethyl cellulose, dextran, poly(ethylene oxide)—poly(propylene oxide) block copolymers as well as blends, association complexes and crosslinked compositions thereof) and films (degradable polyesters, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X—Y, Y—X—Y, R—(Y—X)ₙ, R—(X—Y)ₙ and X—Y—X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator, hyaluronic acid, carboxymethyl cellulose, dextran, poly(ethylene oxide)—poly(propylene oxide) block copolymers as well as blends, association complexes and crosslinked compositions thereof.

A preferred polymeric matrix which can be used to help prevent the formation of fibrous tissue that leads to surgical adhesions, either alone or in combination with a fibrosis inhibiting agent/composition, is formed from reactants comprising either one or both of pentaerythritol poly(ethylene glycol)ether tetra-sulfhydryl] (4-armed thiol PEG, which includes structures having a linking group(s) between a sulfhydryl group(s) and the terminus of the polyethylene glycol backbone) and pentaerythritol poly(ethylene glycol)ether tetra-succinimidyl glutarate] (4-armed NHS PEG, which again includes structures having a linking group(s) between a NHS group(s) and the terminus of the polyethylene glycol backbone) as reactive reagents. Another preferred composition comprises either one or both of pentaerythritol poly(ethylene glycol)ether tetra-amino] (4-armed amino PEG, which includes structures having a linking group(s) between an amino group(s) and the terminus of the polyethylene glycol backbone) and pentaerythritol poly(ethylene glycol)ether tetra-succinimidyl glutarate] (4-armed NHS PEG, which again includes structures having a linking group(s) between a NHS group(s) and the terminus of the polyethylene glycol backbone) as reactive reagents. Chemical structures for these reactants are shown in, e.g., U.S. Pat. No. 5,874,500. Optionally, collagen or a collagen derivative (e.g., methylated collagen) is added to the poly(ethylene glycol)-containing reactant(s) to form a preferred crosslinked matrix that can serve as a polymeric carrier for a therapeutic agent or a stand-alone composition to help prevent the formation of fibrous tissue.

Other examples of polymeric compositions that can be infiltrated into the spinal or neurosurgical site (e.g., onto tissue at the surgical site or in the vicinity of the implant-tissue interface) with or without an additional fibrosis-inhibiting (and/or an anti-infective) therapeutic agent for the prevention of surgical adhesions, include a variety of commercial products. For example, Confluent Surgical, Inc. makes their DURASEAL which is a synthetic hydrogel designed to augment sutured dura closures following cranial surgical procedures. Products that are being developed by Confluent Surgical, Inc. are described in, for example, U.S. Pat. No. 6,379,373. FzioMed, Inc. (San Luis Obispo, Calif.,) makes OXIPLEX/SP Gel which is being sold as an adhesion barrier for spine surgery. OXIPLEX/SP Gel is being used for the reduction of pain and radiculopathy in laminectomy, laminotomy and discectomy surgeries. Products being developed by FzioMed, Inc. are described in, for example, U.S. Pat. Nos. 6,566,345 and 6,017,301. Anika Therapeutics, Inc. (Woburn, Mass.) is developing INCERT-S for the prevention of internal adhesions or scarring following spinal surgery. INCERT-S is part of a potential family of bioabsorbable, chemically modified hyaluronic acid therapies. Products being developed by Anika Therapeutics, Inc. are described in, for example, U.S. Pat. Nos. 6,548,081; 6,537,979; 6,096,727; 6,013,679; 5,502,081 and 5,356,883. Life Medical Sciences, Inc. (Little Silver, N.J.) is developing RELIEVE as a bio-resorbable polymer designed to prevent or reduce the formation of adhesions that can follow spinal surgery. Products being developed by Life Medical Sciences, Inc. are described in, for example, U.S. Pat. Nos. 6,696,499; 6,399,624; 6,211,249; 6,136,333 and 5,711,958. Wright Medical Technology, Inc. is selling the ADCON range of products which are dextran sulfate gels originally developed by Gliatech, Inc. (Beachwood, Ohio) to inhibit postsurgical peridural fibrosis that occurs in posterior lumbar laminectomy or laminotomy procedures where nerve routes are exposed. ADCON provides a barrier between the spinal cord and nerve roots and the surrounding muscle and bone following lumbar spine surgeries. The ADCON range of products may be described in, for example, U.S. Pat. Nos. 6,417,173; 6,127,348; 6,083,930; 5,994,325 and 5,705,178.

Other commercially available materials that may be used alone or loaded with a therapeutic agent (e.g., a fibrosis-inhibiting agent and/or an anti-infective agent), applied to or infiltrated into a spinal or neurosurgical site (or to an implant surface) for the prevention of adhesions include: (a) sprayable collagen-containing formulations such as COSTASIS or CT3; (b) sprayable PEG-containing formulations such as COSEAL, ADHIBIT, FOCALSEAL, or SPRAYGEL; (c) fibrinogen-containing formulations such as FLOSEAL or TISSEAL (both from Baxter Healthcare Corporation, Fremont, Calif.); (d) hyaluronic acid-containing formulations such as RESTYLANE, PERLANE, HYLAFORM, SYNVISC, SEPRAFILM or SEPRACOAT; (e) polymeric gels for surgical implantation such as REPEL or FLOWGEL; (f) surgical adhesives containing cyanoacrylates such as DERMABOND, INDERMIL, GLUSTITCH, TISSUMEND, VETBOND, HISTOACRYL BLUE and ORABASE SOOTHE-N-SEAL LIQUID PROTECTANT; (h) lipid based compositions such as ADSURF, and (j) film compositions such as INTERCEED (Ethicon, Inc., Somerville, N.J.) and HYDROSORB (MacroPore Biosurgery, Inc., San Diego, Calif./Medtronic Sofamor Danek, Memphis, Tenn.). It should be obvious to one of skill in the art that commercial compositions not specifically cited above as well as next-generation and/or subsequently-developed commercial products are to be anticipated and are suitable for use under the present invention.

As described above, the compositions for the prevention of surgical adhesions can be applied directly or indirectly to the tissue in a spinal or neurosurgical site. The polymeric compositions (either with or without a therapeutic agent) can be administered in any manner described herein. Exemplary methods include either direct application at the time of surgery, with endoscopic, ultrasound, Conn., MRI, or fluoroscopic guidance, and/or in conjunction with the placement of a device or implant at the surgical site. Representative examples of devices or implants for use in spinal and neurosurgical procedures includes, without limitation, dural patches, spinal prostheses (e.g., artificial discs, injectable filling or bulking agents for discs, spinal grafts, spinal nucleus implants, intervertebral disc spacers), fusion cages, neurostimulation devices, implantable drug-delivery pumps, shunts, drains, electrodes, and bone fixation devices (e.g., anchoring plates and bone screws).

The polymeric composition, with or without a fibrosis-inhibiting agent, may be applied during open or endoscopic procedures: (a) to the surface of the operative site (e.g., as an injectable, solution, paste, gel, in situ forming gel or mesh) before, during, or after the surgical procedure; (b) to the surface of the tissue surrounding the operative site (e.g., as an injectable, solution, paste, gel, in situ forming gel or mesh) before, during or after the surgical procedure; (c) by topical application of the composition into an anatomical space (such as the subdural space or intrathecally) at the surgical site (particularly useful for this embodiment is the use of polymeric carriers which release the fibrosis-inhibiting agent over a period ranging from several hours to several weeks—fluids, suspensions, emulsions, microemulsions, microspheres, pastes, gels, microparticulates, sprays, aerosols, solid implants and other formulations which release the agent and can be delivered into the region where the device will be inserted); (d) via percutaneous injection into the tissue in and around the operative site as a solution, as an infusate, or as a sustained release preparation; and/or (e) by any combination of the aforementioned methods. Combination therapies (i.e., combinations of therapeutic agents and combinations with antithrombotic, anti-infective, and/or antiplatelet agents) can also be used.

In certain applications involving the placement of a medical device or implant, it may be desirable to apply the anti-fibrosis (and/or anti-infective) composition at a site that is adjacent to an implant (preferably near the implant-tissue interface). This can be accomplished during open or endoscopic procedures by applying the polymeric composition, with or without a fibrosis-inhibiting agent: (a) to the implant surface (e.g., as an injectable, solution, paste, gel, in situ forming gel, or mesh) before, during, or after the implantation procedure; (b) to the surface of the adjacent tissue (e.g., as an injectable, solution, paste, gel, in situ forming gel, or mesh) immediately prior to, during, or after implantation of the implant; (c) to the surface of the implant and the tissue surrounding the implant (e.g., as an injectable, solution, paste, gel, in situ forming gel or mesh) before, during, or after implantation of the implant; (d) by topical application of the composition into the anatomical space (such as the sudural space or intrathecally) where the implant will be placed (particularly useful for this embodiment is the use of polymeric carriers which release the fibrosis-inhibiting agent over a period ranging from several hours to several weeks—fluids, suspensions, emulsions, microemulsions, microspheres, pastes, gels, microparticulates, sprays, aerosols, solid implants and other formulations which release the agent and can be delivered into the region where the device will be inserted); (e) via percutaneous injection into the tissue surrounding the implant as a solution, as an infusate, or as a sustained release preparation; and/or (f) by any combination of the aforementioned methods. Combination therapies (i.e., combinations of therapeutic agents and combinations with antithrombotic, anti-infective, and/or antiplatelet agents) can also be used.

In one aspect, the polymeric composition may be delivered to the tissue (or device/tissue interface) in the form of a spray or gel during open, endoscopic or catheter-based procedures. The fibrosis-inhibiting agent can be incorporated directly into the surgical adhesion barrier or it can be incorporated into a secondary carrier (polymeric or non-polymeric), as described above, that is then incorporated into the adhesion barrier. Examples of polymer compositions that may be in the form of a spray or gel include poly(ethylene glycol)-based systems, hyaluronic acid and crosslinked hyaluronic acid compositions. These compositions can be applied as the final composition or they can be applied as materials that form a crosslinked gel in situ.

In another aspect, an activated polymer is dissolved in a biologically acceptable buffer that has a pH lower that 6.8. The resultant solution is then applied to the desired tissue surface in the presence of a second biologically acceptable buffer that has a pH greater than 7.5. Application of the reaction mixture to the tissue site may be by extrusion, brushing, spraying or by any other convenient means. Following application of the composition to the surgical site, any excess solution may be removed from the surgical site if deemed necessary. At this point in time, the surgical site can be closed using conventional means (e.g., sutures, staples, or a bioadhesive). In one embodiment, the activated polymer can form a covalent bond with the tissue to which it is applied may be used. Polymers containing and/or terminated with electrophilic groups such as succinimidyl, aldehyde, epoxide, isocyanate, vinyl, vinyl sulfone, maleimide, —S—S—(C₅H₄N) or activated esters, such as are used in peptide synthesis may be used as the reagents. For example, a 4 armed NHS-derivatized polyethylene glycol (e.g., pentaerythritol poly(ethylene glycol)ether tetra-succinimidyl glutarate) may be applied to the tissue in the solid form or in a solution form. In this embodiment, the 4 armed NHS-derivatized polyethylene glycol is dissolved in an acidic solution (pH about 2-3) and is then co-applied to the tissue using a basic buffer (pH>about 8). The antifibrosisfibrosis-inhibiting agent(s) may be incorporated directly into either the 4 armed NHS-derivatized polyethylene glycol, the acidic solution or the basic buffer. In another embodiment, the fibrosis-inhibiting agent may be incorporated into a secondary carrier that may then be incorporated into the 4 armed NHS-derivatized polyethylene glycol, the acidic solution and/or the basic buffer. The secondary carriers may include microparticles and/or microspheres which are made from degradable polymers. The degradable polymers may include polyesters, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X—Y, Y—X—Y, R—(Y—X)ₙ, R—(X—Y)ₙ and X—Y—X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator. In another embodiment, the tissue reactive polymer may be applied initially and then the fibrosis-inhibiting agent may then be applied to the coated tissue. The fibrosis-inhibiting agent may be applied directly to the tissue or it may be incorporated into a secondary carrier. The secondary carriers may include microspheres (as described above), microparticles (as described above), gels (e.g., hyaluronic acid, carboxymethyl cellulose, dextran, poly(ethylene oxide)—poly(propylene oxide) block copolymers as well as blends, association complexes and crosslinked compositions thereof) and films (degradable polyesters, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X—Y, Y—X—Y, R—(Y—X)ₙ, R—(X—Y)ₙ and X—Y—X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator, hyaluronic acid, carboxymethyl cellulose, dextran, poly(ethylene oxide)—poly(propylene oxide) block copolymers as well as blends, association complexes and crosslinked compositions thereof.

In yet another aspect, an activated polymer can be applied to the surgical site in the solid state. The activated polymer can react with the tissue surface to which it was applied as the polymer hydrates. A biologically acceptable buffer, with a pH greater than 7.5 can be applied to the tissue before and/or after the solid activated polymer has been applied. In one embodiment, the activated polymer can form a covalent bond with the tissue to which it is applied may be used. Polymers containing and/or terminated with electrophilic groups such as succinimidyl, aldehyde, epoxide, isocyanate, vinyl, vinyl sulfone, maleimide, —S—S—(C₅H₄N) or activated esters, such as are used in peptide synthesis may be used as the reagents. For example, a 4 armed NHS-derivatized polyethylene glycol (e.g., pentaerythritol poly(ethylene glycol)ether tetra-succinimidyl glutarate) may be applied to the tissue in the solid form. The antifibrosisfibrosis-inhibiting agent(s) may be incorporated directly into either the 4 armed NHS-derivatized polyethylene glycol, or the basic buffer. In another embodiment, the fibrosis-inhibiting agent may be incorporated into a secondary carrier that may then be incorporated into the 4 armed NHS-derivatized polyethylene glycol, and/or the basic buffer. The secondary carriers may include microparticles and/or microspheres which are made from degradable polymers. The degradable polymers may include polyesters, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X—Y, Y—X—Y, R—(Y—X)ₙ, R—(X—Y)ₙ and X—Y—X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator. In another embodiment, the tissue reactive polymer may be applied initially and then the fibrosis-inhibiting agent may then be applied to the coated tissue. The fibrosis-inhibiting agent may be applied directly to the tissue or it may be incorporated into a secondary carrier. The secondary carriers may include microspheres (as described above), microparticles (as described above), gels (e.g., hyaluronic acid, carboxymethyl cellulose, dextran, poly(ethylene oxide)—poly(propylene oxide) block copolymers as well as blends, association complexes and crosslinked compositions thereof) and films (degradable polyesters, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X—Y, Y—X—Y, R—(Y—X)ₙ, R—(X—Y)ₙ and X—Y—X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator, hyaluronic acid, carboxymethyl cellulose, dextran, poly(ethylene oxide)—poly(propylene oxide) block copolymers as well as blends, association complexes and crosslinked compositions thereof

ii) Adhesion Prevention in Gynecological Procedures

In one aspect, adhesion formation may be associated with a gynecological surgical procedure. The post-operative adhesions occur in 60 to 90% of patients undergoing major gynecologic surgery and represent one of the most common causes of infertility in the industrialized world. Adhesions can form between the ovaries, the fallopian tubes, the bowel or the walls of the pelvis. Fibrous bands can connect to the normally mobile adnexal structures (ovaries and fallopian tubes) to other tissues, causing them to lose mobility, kink or twist. If the adhesions tighten around, constrict or twist the fallopian tubes themselves, they can block the passage of an ovum from the ovaries into and through the fallopian tube leading to infertility. Adhesions around the fallopian tubes can also interfere with sperm transport to the ovum and also cause infertility. Other adhesion-related complications include chronic pelvic pain, dysparunia, urethral obstruction and voiding dysfunction.

Several products are available commercially or under development for the management of gynecological adhesions. Life Medical Sciences, Inc. is producing the products, REPEL, REPEL-CV, RESOLVE and RELIEVE that are in various stages of development and may be used to prevent surgical adhesions in gynecological and other surgeries. Products being developed by Life Medical Sciences, Inc. are described in, for example, U.S. Pat. Nos. 6,696,499; 6,399,624; 6,211,249; 6,136,333 and 5,711,958. Confluent Surgical, Inc. makes their SPRAYGEL which is a unique sprayable adhesion barrier that is being developed for use in pelvic and intrauterine surgical procedures. Products that are being developed by Confluent Surgical, Inc. are described in, for example, U.S. Pat. No. 6,379,373. Closure Medical Corp. (Raleigh, N.C.) is developing a cyanoacrylate-based internal adhesives that may be used to seal internal surgical incisions or grafts which may be compatible in gynecology and general surgical specialties. Products that are being developed by Closure Medical, Corp. are described in, for example, U.S. Pat. Nos. 6,620,846; 6,579,469; 6,565,840; 6,547,467 and 5,981,621.

Other commercially available materials that may be used alone, or loaded with a therapeutic agent (e.g., a fibrosis-inhibiting agent and/or an anti-infective agent), applied to or infiltrated into a gynecological surgical site (or to the surface of a device or implant) for the prevention of adhesions in open or endoscopic gynecologic surgery include: (a) sprayable collagen-containing formulations such as COSTASIS or CT3; (b) sprayable PEG-containing formulations such as COSEAL, ADHIBIT, FOCALSEAL or DURASEAL; (c) fibrinogen-containing formulations such as FLOSEAL or TISSEAL; (d) hyaluronic acid-containing formulations such as RESTYLANE or PERLANE, HYLAFORM, SYNVISC, SEPRAFILM or SEPRACOAT; (e) polymeric gels for surgical implantation such as FLOWGEL; (f) surgical adhesives containing cyanoacrylates such as DERMABOND, INDERMIL, GLUSTITCH, TISSUMEND, VETBOND, HISTOACRYL BLUE and ORABASE SOOTHE-N-SEAL LIQUID PROTECTANT; (g) dextran sulfate gels such as the ADCON series of gels; and (h) lipid based compositions such as ADSURF. It should be obvious to one of skill in the art that commercial compositions not specifically cited above as well as next-generation and/or subsequently-developed commercial products are to be anticipated and are suitable for use under the present invention.

Gynecological procedures are performed for a variety of medical conditions including hysterectomy (removal of the uterus), myomectomy (removal of uterine fibroids), endometriosis (ablation procedures), infertility (in vitro fertilization, adhesiolysis), birth control (tubal ligation), reversal of sterilization, pain, dysmennorrhea, dysfunctional uterine bleeding, ectopic pregnancy, ovarian cysts, gynecologic malignancies and numerous other conditions. Although many procedures are still performed through open surgical techniques, increasingly, gynecologic surgery is performed via an endoscope inserted through the umbilicus (belly button). Virtually any manipulation of the pelvic organs or pelvic sidewall can trigger a cascade that ultimately results in the formation of pelvic adhesions. In many instances, the adhesions must be broken down during a repeat surgical intervention for the treatment of pain or infertility. An adhesion barrier, either alone or containing a fibrosis-inhibiting agent (and/or an anti-infective agent), is best applied directly to the affected areas (as a solid, a film, a paste, a gel, a liquid or another such formulation) during the open or endoscopic procedure. In a preferred embodiment, the barrier (alone or containing an anti-fibrotic and/or anti-infective agent) is sprayed under direct endoscopic vision during the procedure onto the pelvic organs (and bowel, pelvic and abdominal sidewall) that are operated on, or manipulated, during the intervention. Since adhesions often occur in areas at a distance from the tissues actually instrumented during a surgical intervention, it is recommended that the barrier (with or without a therapeutic agent) be applied to a wide area in the pelvis (potentially even the entire adnexa, pelvic sidewall and pelvic surface of the uterus). Preferred barriers include liquids, gels, pastes, sprays or other formulations that can be delivered through an endoscope, adhere to the tissues treated, and remain in place long enough to deliver the therapeutic agent and/or prevent adhesion formation. As an alternative, the therapeutic agent can be delivered directly into the peritoneal cavity as an injectable (either before, during or after the procedure) such that the drug is delivered in doses high enough and long enough (multiple dosing and/or sustained release preparations are preferred) to prevent adhesions and the complications arising from them. An ideal adhesion therapy will reduce the incidence, number and tenacity of adhesions and improve patient outcome by reducing pain, improving fertility and limiting the need for repeat interventions.

As described above, the compositions for the prevention of surgical adhesions can be applied directly or indirectly to the tissue in a gynecological site. The polymeric compositions (either with or without an anti-fibrotic or anti-infective therapeutic agent) can be administered in any manner described herein. Exemplary methods include either direct application at the time of surgery or with endoscopic, ultrasound, CT, MRI, or fluoroscopic guidance. If an implanted device is being placed, the composition for the prevention of adhesions can be applied to the surface of the implant, or to the surrounding tissues, in conjunction with placement of a medical device or implant at the surgical site. Representative examples of implants for use in gynecological procedures includes, without limitation, genital-urinary stents, bulking agents, sterilization devices (e.g., valves, clips and clamps), and tubal occlusion implants and plugs.

The polymeric composition, with or without a fibrosis-inhibiting agent, may be applied during open or endoscopic gynecological surgery: (a) to the tissue surface of the pelvic side wall, adnexa, uterus and any adjacent affected tissues (e.g., as an injectable, solution, paste, gel, in situ forming gel or mesh) during the surgical procedure; (b) to the surface of an implanted device or implant and/or the tissue surrounding the implant (e.g., as an injectable, solution, paste, gel, in situ forming gel or mesh) before, during, or after the surgical procedure; (c) by intraperitoneal or endoscopic injection of the composition into the anatomical space (i.e., the peritoneal or pelvic cavity) at the surgical site (particularly useful for this embodiment is the use of injectable compositions containing polymeric carriers which release the fibrosis-inhibiting agent over a period ranging from several hours to several weeks—fluids, suspensions, emulsions, microemulsions, microspheres, pastes, gels, microparticulates, sprays, aerosols, solid implants and other formulations which release the agent and can be delivered into the region where there is a risk of adhesion formation); (d) via percutaneous injection into the tissue as a solution as an infusate or as a sustained release preparation; (e) by guided catheter or hysteroscopic injection of the composition into the lumen of the fallopian tubes (i.e., inserting a catheter or an endoscope via the vagina, cervix and uterus until it can be advanced into the lumen of the fallopian tube) at the desired tubal location (particularly useful for this embodiment is the use of injectable compositions containing polymeric carriers which release the fibrosis-inhibiting agent over a period ranging from several hours to several weeks—fluids, suspensions, emulsions, microemulsions, microspheres, pastes, gels, microparticulates, sprays, aerosols, solid implants and other formulations which release the agent can be delivered into the areas of the fallopian tube where there is a risk of adhesion formation); and/or (f) by any combination of the aforementioned methods. Combination therapies (i.e., combinations of therapeutic agents and combinations with antithrombotic, anti-infective, and/or antiplatelet agents) can also be used in the manner described above.

In certain applications involving the placement of a gynecological medical device or implant, it may be desirable to apply the anti-fibrosis (and/or anti-infective) composition at a site that is adjacent to an implant (preferably near the implant-tissue interface). This can be accomplished during open or endoscopic procedures by applying the polymeric composition, with or without a fibrosis-inhibiting agent: (a) to the implant surface (e.g., as an injectable, solution, paste, gel, in situ forming gel, or mesh) before, during, or after the implantation procedure; (b) to the surface of the adjacent tissue (e.g., as an injectable, solution, paste, gel, in situ forming gel, or mesh) immediately prior to, during, or after implantation of the implant; (c) to the surface of the implant and the tissue surrounding the implant (e.g., as an injectable, solution, paste, gel, in situ forming gel or mesh) before, during, or after implantation of the implant; (d) by topical application of the composition into the anatomical space (such as the lumen of the fallopian tube, the uterine cavity, the peritoneal cavity, or the pelvic cavity) where the implant will be placed (particularly useful for this embodiment is the use of polymeric carriers which release the fibrosis-inhibiting agent over a period ranging from several hours to several weeks—fluids, suspensions, emulsions, microemulsions, microspheres, pastes, gels, microparticulates, sprays, aerosols, solid implants and other formulations which release the agent and can be delivered into the region where the device will be inserted); (e) via percutaneous injection into the tissue surrounding the implant as a solution, as an infusate, or as a sustained release preparation; and/or (f) by any combination of the aforementioned methods. Combination therapies (i.e., combinations of therapeutic agents and combinations with antithrombotic, anti-infective, and/or antiplatelet agents) can also be used.

In one aspect, the polymeric composition may be delivered to the female pelvic tissue (or device/tissue interface) in the form of a spray or gel during open, endoscopic or catheter-based procedures. The fibrosis-inhibiting agent can be incorporated directly into the surgical adhesion barrier or it can be incorporated into a secondary carrier (polymeric or non-polymeric), as described above, that is then incorporated into the adhesion barrier. Examples of polymer compositions that may be in the form of a spray or gel include poly(ethylene glycol)-based systems, hyaluronic acid and crosslinked hyaluronic acid compositions. These compositions can be applied as the final composition or they can be applied as materials that form a crosslinked gel in situ.

In another aspect, an activated polymer is dissolved in a biologically acceptable buffer that has a pH lower that 6.8. The resultant solution is then applied to the desired tissue surface in the presence of a second biologically acceptable buffer that has a pH greater than 7.5. Application of the reaction mixture to the tissue site may be by extrusion, brushing, spraying or by any other convenient means. Following application of the composition to the surgical site, any excess solution may be removed from the surgical site if deemed necessary. At this point in time, the surgical site can be closed using conventional means (e.g., sutures, staples, or a bioadhesive). In one embodiment, the activated polymer can form a covalent bond with the tissue to which it is applied may be used. Polymers containing and/or terminated with electrophilic groups such as succinimidyl, aldehyde, epoxide, isocyanate, vinyl, vinyl sulfone, maleimide, —S—S—(C₅H₄N) or activated esters, such as are used in peptide synthesis may be used as the reagents. For example, a 4 armed NHS-derivatized polyethylene glycol (e.g., pentaerythritol poly(ethylene glycol)ether tetra-succinimidyl glutarate) may be applied to the tissue in the solid form or in a solution form. In this embodiment, the 4 armed NHS-derivatized polyethylene glycol is dissolved in an acidic solution (pH about 2-3) and is then co-applied to the tissue using a basic buffer (pH>about 8). The antifibrosisfibrosis-inhibiting agent(s) may be incorporated directly into either the 4 armed NHS-derivatized polyethylene glycol, the acidic solution or the basic buffer. In another embodiment, the fibrosis-inhibiting agent may be incorporated into a secondary carrier that may then be incorporated into the 4 armed NHS-derivatized polyethylene glycol, the acidic solution and/or the basic buffer. The secondary carriers may include microparticles and/or microspheres which are made from degradable polymers. The degradable polymers may include polyesters, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X—Y, Y—X—Y, R—(Y—X)ₙ, R—(X—Y)ₙ and X—Y—X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator. In another embodiment, the tissue reactive polymer may be applied initially and then the fibrosis-inhibiting agent may then be applied to the coated tissue. The fibrosis-inhibiting agent may be applied directly to the tissue or it may be incorporated into a secondary carrier. The secondary carriers may include microspheres (as described above), microparticles (as described above), gels (e.g., hyaluronic acid, carboxymethyl cellulose, dextran, poly(ethylene oxide)—poly(propylene oxide) block copolymers as well as blends, association complexes and crosslinked compositions thereof) and films (degradable polyesters, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X—Y, Y—X—Y, R—(Y—X)ₙ, R—(X—Y)ₙ and X—Y—X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator, hyaluronic acid, carboxymethyl cellulose, dextran, poly(ethylene oxide)—poly(propylene oxide) block copolymers as well as blends, association complexes and crosslinked compositions thereof.

In yet another aspect, an activated polymer can be applied to the surgical site in the solid state. The activated polymer can react with the tissue surface to which it was applied as the polymer hydrates. A biologically acceptable buffer, with a pH greater than 7.5 can be applied to the tissue before and/or after the solid activated polymer has been applied. In one embodiment, the activated polymer can form a covalent bond with the tissue to which it is applied may be used. Polymers containing and/or terminated with electrophilic groups such as succinimidyl, aldehyde, epoxide, isocyanate, vinyl, vinyl sulfone, maleimide, —S—S—(C₅H₄N) or activated esters, such as are used in peptide synthesis may be used as the reagents. For example, a 4 armed NHS-derivatized polyethylene glycol (e.g., pentaerythritol poly(ethylene glycol)ether tetra-succinimidyl glutarate) may be applied to the tissue in the solid form. The antifibrosisfibrosis-inhibiting agent(s) may be incorporated directly into either the 4 armed NHS-derivatized polyethylene glycol, or the basic buffer. In another embodiment, the fibrosis-inhibiting agent may be incorporated into a secondary carrier that may then be incorporated into the 4 armed NHS-derivatized polyethylene glycol, and/or the basic buffer. The secondary carriers may include microparticles and/or microspheres which are made from degradable polymers. The degradable polymers may include polyesters, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X—Y, Y—X—Y, R—(Y—X)ₙ, R—(X—Y)ₙ and X—Y—X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator. In another embodiment, the tissue reactive polymer may be applied initially and then the fibrosis-inhibiting agent may then be applied to the coated tissue. The fibrosis-inhibiting agent may be applied directly to the tissue or it may be incorporated into a secondary carrier. The secondary carriers may include microspheres (as described above), microparticles (as described above), gels (e.g., hyaluronic acid, carboxymethyl cellulose, dextran, poly(ethylene oxide)—poly(propylene oxide) block copolymers as well as blends, association complexes and crosslinked compositions thereof) and films (degradable polyesters, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X—Y, Y—X—Y, R—(Y—X)ₙ, R—(X—Y)ₙ and X—Y—X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator, hyaluronic acid, carboxymethyl cellulose, dextran, poly(ethylene oxide)—poly(propylene oxide) block copolymers as well as blends, association complexes and crosslinked compositions thereof.

iii) Adhesion Prevention in Abdominal Procedures

In one aspect, adhesions may be associated with an abdominal surgical procedure. Following abdominal surgery, the formation of adhesions may cause loops of intestines become entangled or twisted about fibrous bands of tissue that impair the normal fluid movement of the bowel. The entanglements can cause partial or total flow obstruction through the bowel, scar can constrict around the bowel, volvulus (twisting) can occur, or blood flow to and from the bowel can be compromised. With entanglement, volvulus or fibrous banding the result is typically partial or complete bowel obstruction; a condition that requires immediate decompression, may require surgery and can cause death. Infarction (interruption of blood flow to the bowel) from adhesions or volvulus is a medical emergency that usually requires surgical removal of the affected bowel and can also lead to death if not treated aggressively. Peritoneal adhesions (adhesions between the abdominal wall and the underlying organs) represent another major health care problem causing pain, bowel obstruction and other potentially serious post-operative complications and they are associated with all types of abdominal surgery (incidence of 50-90% for laparotomies).

As described previously, adhesion barriers are frequently used in the management of abdominal adhesions following open or endoscopic procedures. A variety of commercially available adhesion barriers are suitable for combining with a fibrosis-inhibitor (and/or an anti-infective agent) in the management of abdominal adhesions. Confluent Surgical, Inc. makes their SPRAYGEL which is a unique sprayable adhesion barrier that is being developed for use in abdominal and pelvic surgical procedures. Products that are being developed by Confluent Surgical, Inc. are described in, for example, U.S. Pat. No. 6,379,373. Closure Medical Corp. (Raleigh, N.C.) is developing a cyanoacrylate-based internal adhesives that may be used to seal internal surgical incisions or grafts which may be compatible in gastrointestinal, oncology and general surgical specialties. Products that are being developed by Closure Medical, Corp. are described in, for example, U.S. Pat. Nos. 6,620,846; 6,579,469; 6,565,840; 6,547,467 and 5,981,621. Genzyme Corporation has developed hyaluronic acid-containing biomaterials, such as SEPRAFILM and SEPRACOAT, to reduce the incidence of adhesions following abdominal and pelvic surgeries (see, e.g., U.S. Pat. Nos. 6,780,427; 6,531,147; 6,521,223 and 6,010,692.

Other commercially available materials that may be used alone, or loaded with a therapeutic agent (e.g., a fibrosis-inhibiting agent or an anti-infective agent), applied to or infiltrated into an abdominal site (or to the surface of an implanted device or implant) for the prevention of adhesions during open or endoscopic abdominal procedures include: (a) sprayable collagen-containing formulations such as COSTASIS or CT3; (b) sprayable PEG-containing formulations such as COSEAL, ADHIBIT, FOCALSEAL or DURASEAL; (c) fibrinogen-containing formulations such as FLOSEAL or TISSEAL; (d) hyaluronic acid-containing formulations such as RESTYLANE or PERLANE, HYLAFORM, or SYNVISC; (e) polymeric gels for surgical implantation such as REPEL or FLOWGEL; (f) surgical adhesives containing cyanoacrylates such as DERMABOND, INDERMIL, GLUSTITCH, TISSUMEND, VETBOND, HISTOACRYL BLUE and ORABASE SOOTHE-N-SEAL LIQUID PROTECTANT; (g) dextran sulfate gels such as the ADCON series of gels; and (h) lipid based compositions such as ADSURF. It should be obvious to one of skill in the art that commercial compositions not specifically cited above as well as next-generation and/or subsequently-developed commercial products are to be anticipated and are suitable for use under the present invention.

Abdominal surgical procedures are performed for a variety of medical conditions including hernia repair (abdominal, ventral, inguinal, incisional), bowel obstruction, inflammatory bowel disease (ulcerative colitis, Crohn's disease), appendectomy, trauma (penetrating wounds, blunt tauma), tumor resection, infections (abscesses, peritonitis), cholecystectomy, gastroplasty (bariatric surgery), esophageal and pyloric strictures, colostomy, diversion iliostomy, anal-rectal fistulas, hemorrhoidectomies, splenectomy, hepatic tumor resection, pancreatitis, bowel perforation, upper and lower GI bleeding, and ischemic bowel. Although many procedures are still performed through open surgical techniques, increasingly, abdominal surgery is performed via an endoscope inserted through the umbilicus (belly button). Virtually any manipulation of the abdominal viscera or peritoneum can trigger a cascade that ultimately results in the formation of abdominal adhesions. In many instances, the adhesions must be broken down during a repeat surgical intervention for the treatment of pain or bowel obstruction. An adhesion barrier, either alone or containing a fibrosis-inhibiting agent (and/or an anti-infective agent), is best applied directly to the affected areas (as a solid, a film, a paste, a gel, a liquid or another such formulation) during the open or endoscopic procedure. In a preferred embodiment, the barrier (alone or containing an anti-fibrotic and/or anti-infective agent) is sprayed under direct or endoscopic vision during the procedure onto the abdominal organs (such as the large and small bowel, stomach, liver, spleen, gall bladder etc.), visceral peritoneum and abdominal (wall) peritoneum that are operated on, or manipulated, during the intervention. Since adhesions often occur in areas at a distance from the tissues actually instrumented during a surgical intervention, it is recommended that the barrier (with or without a therapeutic agent) be applied to a wide area in the abdomen (potentially even the entire viscera and abdominal wall). Preferred barriers include films, liquids, gels, pastes, sprays or other formulations that can be delivered during open procedures or through an endoscope, adhere to the tissues treated, and remain in place long enough to deliver the therapeutic agent and/or prevent adhesion formation. As an alternative, the therapeutic agent can be delivered directly into the peritoneal cavity as an injectable (either before, during or after the procedure) such that the drug is delivered in doses high enough and long enough (multiple dosing and/or sustained release preparations are preferred) to prevent adhesions and the complications arising from them. An ideal adhesion therapy will reduce the incidence, number and tenacity of adhesions and improve patient outcome by reducing pain, preventing bowel obstruction and limiting the need for repeat interventions.

As described above, the compositions for the prevention of surgical adhesions can be applied directly or indirectly to the tissue in an abdominal procedure. The polymeric compositions (either with or without an anti-fibrotic or anti-infective therapeutic agent) can be administered in any manner described herein. Exemplary methods include either direct application at the time of surgery or with endoscopic, ultrasound, CT, MRI, or fluoroscopic guidance. If an implanted device is being placed, the composition for the prevention of adhesions can be applied to the surface of the implant, or to the surrounding tissues, in conjunction with placement of a medical device or implant at the surgical site. Representative examples of implants for use in abdominal procedures includes, without limitation, hernia meshes, restriction devices for obesity, implantable sensors, implantable pumps, peritoneal dialysis catheters, peritoneal drug-delivery catheters, GI tubes for drainage or feeding, portosystemic shunts, shunts for ascites, gastrostomy or percutaneous feeding tubes, jejunostomy endoscopic tubes, colostomy devices, drainage tubes, biliary T-tubes, hemostatic implants, enteral feeding devices, colonic and biliary stents, low profile devices, gastric banding implants, capsule endoscopes, anti-reflux devices, and esophageal stents.

The polymeric composition, with or without a fibrosis-inhibiting agent, may be applied during open or endoscopic abdominal surgery: (a) to the tissue surface of the peritoneal cavity, visceral peritneum, abdominal organs, abdominal wall and any adjacent affected tissues (e.g., as an injectable, solution, paste, gel, in situ forming gel or mesh) during the surgical procedure; (b) to the surface of an implanted device or implant and/or the tissue surrounding the implant (e.g., as an injectable, solution, paste, gel, in situ forming gel or mesh) before, during, or after the surgical procedure; (c) by intraperitoneal or endoscopic injection of the composition into the anatomical space (i.e., the peritoneal cavity) at the surgical site (particularly useful for this embodiment is the use of injectable compositions containing polymeric carriers which release the fibrosis-inhibiting agent over a period ranging from several hours to several weeks—fluids, suspensions, emulsions, microemulsions, microspheres, pastes, gels, microparticulates, sprays, aerosols, solid implants and other formulations which release the agent and can be delivered into the region where there is a risk of adhesion formation); (d) via percutaneous injection into the tissue as a solution as an infusate or as a sustained release preparation; (e) by guided catheter or endoscopic (gastroscope, ERCP, colonoscope) injection of the composition into the lumen of the GI tract at the desired location (particularly useful for this embodiment is the use of injectable compositions containing polymeric carriers which release the fibrosis-inhibiting agent over a period ranging from several hours to several weeks—fluids, suspensions, emulsions, microemulsions, microspheres, pastes, gels, microparticulates, sprays, aerosols, solid implants and other formulations which release the agent can be delivered into the areas of the GI tract where there is a risk of adhesion formation); and/or (f) by any combination of the aforementioned methods. Combination therapies (i.e., combinations of therapeutic agents and combinations with antithrombotic, anti-infective, and/or antiplatelet agents) can also be used in the manner described above.

In certain applications involving the placement of an abdominal or gastrointestinal medical device or implant, it may be desirable to apply the anti-fibrosis (and/or anti-infective) composition at a site that is adjacent to an implant (preferably near the implant-tissue interface). This can be accomplished during open or endoscopic procedures by applying the polymeric composition, with or without a fibrosis-inhibiting agent: (a) to the implant surface (e.g., as an injectable, solution, paste, gel, in situ forming gel, or mesh) before, during, or after the implantation procedure; (b) to the surface of the adjacent tissue (e.g., as an injectable, solution, paste, gel, in situ forming gel, or mesh) immediately prior to, during, or after implantation of the implant; (c) to the surface of the implant and the tissue surrounding the implant (e.g., as an injectable, solution, paste, gel, in situ forming gel or mesh) before, during, or after implantation of the implant; (d) by topical application of the composition into the anatomical space (such as the lumen of the GI tract or the peritoneal cavity) where the implant will be placed (particularly useful for this embodiment is the use of polymeric carriers which release the fibrosis-inhibiting agent over a period ranging from several hours to several weeks—fluids, suspensions, emulsions, microemulsions, microspheres, pastes, gels, microparticulates, sprays, aerosols, solid implants and other formulations which release the agent and can be delivered into the region where the device will be inserted); (e) via percutaneous injection into the tissue surrounding the implant as a solution, as an infusate, or as a sustained release preparation; and/or (f) by any combination of the aforementioned methods. Combination therapies (i.e., combinations of therapeutic agents and combinations with antithrombotic, anti-infective, and/or antiplatelet agents) can also be used.

In one aspect, the polymeric composition may be delivered to the abdomen (or device/tissue interface) in the form of a spray or gel during open, endoscopic or catheter-based procedures. The fibrosis-inhibiting agent can be incorporated directly into the surgical adhesion barrier or it can be incorporated into a secondary carrier (polymeric or non-polymeric), as described above, that is then incorporated into the adhesion barrier. Examples of polymer compositions that may be in the form of a spray or gel include poly(ethylene glycol)-based systems, hyaluronic acid and crosslinked hyaluronic acid compositions. These compositions can be applied as the final composition or they can be applied as materials that form a crosslinked gel in situ.

In another aspect, an activated polymer is dissolved in a biologically acceptable buffer that has a pH lower that 6.8. The resultant solution is then applied to the desired tissue surface in the presence of a second biologically acceptable buffer that has a pH greater than 7.5. Application of the reaction mixture to the tissue site may be by extrusion, brushing, spraying or by any other convenient means. Following application of the composition to the surgical site, any excess solution may be removed from the surgical site if deemed necessary. At this point in time, the surgical site can be closed using conventional means (e.g., sutures, staples, or a bioadhesive). In one embodiment, the activated polymer can form a covalent bond with the tissue to which it is applied may be used. Polymers containing and/or terminated with electrophilic groups such as succinimidyl, aldehyde, epoxide, isocyanate, vinyl, vinyl sulfone, maleimide, —S—S—(C₅H₄N) or activated esters, such as are used in peptide synthesis may be used as the reagents. For example, a 4 armed NHS-derivatized polyethylene glycol (e.g., pentaerythritol poly(ethylene glycol)ether tetra-succinimidyl glutarate) may be applied to the tissue in the solid form or in a solution form. In this embodiment, the 4 armed NHS-derivatized polyethylene glycol is dissolved in an acidic solution (pH about 2-3) and is then co-applied to the tissue using a basic buffer (pH>about 8). The antifibrosisfibrosis-inhibiting agent(s) may be incorporated directly into either the 4 armed NHS-derivatized polyethylene glycol, the acidic solution or the basic buffer. In another embodiment, the fibrosis-inhibiting agent may be incorporated into a secondary carrier that may then be incorporated into the 4 armed NHS-derivatized polyethylene glycol, the acidic solution and/or the basic buffer. The secondary carriers may include microparticles and/or microspheres which are made from degradable polymers. The degradable polymers may include polyesters, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X—Y, Y—X—Y, R—(Y—X)ₙ, R—(X—Y)ₙ and X—Y—X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator. In another embodiment, the tissue reactive polymer may be applied initially and then the fibrosis-inhibiting agent may then be applied to the coated tissue. The fibrosis-inhibiting agent may be applied directly to the tissue or it may be incorporated into a secondary carrier. The secondary carriers may include microspheres (as described above), microparticles (as described above), gels (e.g., hyaluronic acid, carboxymethyl cellulose, dextran, poly(ethylene oxide)—poly(propylene oxide) block copolymers as well as blends, association complexes and crosslinked compositions thereof) and films (degradable polyesters, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X—Y, Y—X—Y, R—(Y—X)ₙ, R—(X—Y)ₙ and X—Y—X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator, hyaluronic acid, carboxymethyl cellulose, dextran, poly(ethylene oxide)—poly(propylene oxide) block copolymers as well as blends, association complexes and crosslinked compositions thereof.

In yet another aspect, an activated polymer can be applied to the surgical site in the solid state. The activated polymer can react with the tissue surface to which it was applied as the polymer hydrates. A biologically acceptable buffer, with a pH greater than 7.5 can be applied to the tissue before and/or after the solid activated polymer has been applied. In one embodiment, the activated polymer can form a covalent bond with the tissue to which it is applied may be used. Polymers containing and/or terminated with electrophilic groups such as succinimidyl, aldehyde, epoxide, isocyanate, vinyl, vinyl sulfone, maleimide, —S—S—(C₅H₄N) or activated esters, such as are used in peptide synthesis may be used as the reagents. For example, a 4 armed NHS-derivatized polyethylene glycol (e.g., pentaerythritol poly(ethylene glycol)ether tetra-succinimidyl glutarate) may be applied to the tissue in the solid form. The antifibrosisfibrosis-inhibiting agent(s) may be incorporated directly into either the 4 armed NHS-derivatized polyethylene glycol, or the basic buffer. In another embodiment, the fibrosis-inhibiting agent may be incorporated into a secondary carrier that may then be incorporated into the 4 armed NHS-derivatized polyethylene glycol, and/or the basic buffer. The secondary carriers may include microparticles and/or microspheres which are made from degradable polymers. The degradable polymers may include polyesters, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X—Y, Y—X—Y, R—(Y—X)ₙ, R—(X—Y)ₙ and X—Y—X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator. In another embodiment, the tissue reactive polymer may be applied initially and then the fibrosis-inhibiting agent may then be applied to the coated tissue. The fibrosis-inhibiting agent may be applied directly to the tissue or it may be incorporated into a secondary carrier. The secondary carriers may include microspheres (as described above), microparticles (as described above), gels (e.g., hyaluronic acid, carboxymethyl cellulose, dextran, poly(ethylene oxide)—poly(propylene oxide) block copolymers as well as blends, association complexes and crosslinked compositions thereof) and films (degradable polyesters, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X—Y, Y—X—Y, R—(Y—X)ₙ, R—(X—Y)ₙ and X—Y—X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator, hyaluronic acid, carboxymethyl cellulose, dextran, poly(ethylene oxide)—poly(propylene oxide) block copolymers as well as blends, association complexes and crosslinked compositions thereof.

iv) Adhesion Prevention in Cardiac Procedures

In one aspect, adhesions may be associated with a cardiac surgical procedure. In the case of cardiac surgery involving transplants, vascular repair, coronary artery bypass grafting (CABG), congenital heart defects, and valve replacements, staged procedures and reoperations (particularly repeat CABG surgery) are very common. As such, cardiac surgeons frequently must operate on tissues that have been surgically traumatized previously and have thick fibrous adhesions present which make dissection difficult. Post-operative pericardial adhesions (adhesions between the two surfaces of the pericardial sac) from initial surgery are common. Pericardial adhesions can cause symptoms by restricting the normal movement and filling of the heart during the cardiac cycle and can subject patients undergoing repeat cardiac surgery to elevated procedural risks. Resternotomy (re-opening the chest wall incision and surgical exposure of the heart) and dissection of the adhesions that accompany it, increases the risk of potential injury to the heart, great vessels and extracardiac grafts, increases operative time (including increasing the time the patient is on heart-lung bypass), and can increase procedural morbidity and mortality. Resternotomy is associated with as much as a 6% incidence of major vascular injury and a greater than 35% mortality has been reported for patients experiencing major hemorrhage during resternotomy. A 50% mortality has been reported for associated injuries to aortocoronary grafts. Staged pediatric open-heart surgery (repeat procedures required as the heart grows) is also associated with a very high incidence of complications due to reoperations.

As described previously, adhesion barriers are frequently used in the management of adhesions following open-heart procedures. A variety of commercially available adhesion barriers are suitable for combining with a fibrosis-inhibitor (and/or an anti-infective agent) in the management of cardiac surgery adhesions. Life Medical Sciences, Inc. is developing the products, REPEL, REPEL-CV, RESOLVE and RELIEVE that are in various stages of development and may be used to prevent surgical adhesions of open heart and other surgeries. Products being developed by Life Medical Sciences, Inc. are described in, for example, U.S. Pat. Nos. 6,696,499; 6,399,624; 6,211,249; 6,136,333 and 5,711,958. Closure Medical Corp. (Raleigh, N.C.) is developing a cyanoacrylate-based internal adhesives that may be used to seal internal surgical incisions or grafts which may be compatible in pulmonary and general surgical specialties. Products that are being developed by Closure Medical, Corp. are described in, for example, U.S. Pat. Nos. 6,620,846; 6,579,469; 6,565,840; 6,547,467 and 5,981,621. Genzyme Corporation has developed hyaluronic acid-containing biomaterials, such as SEPRAFILM and SEPRACOAT, to reduce the incidence of adhesions following cardiothoracic surgeries (see, e.g., U.S. Pat. Nos. 6,780,427; 6,531,147; 6,521,223 and 6,010,692.

Other commercially available materials that may be used alone, or loaded with a therapeutic agent (e.g., a fibrosis-inhibiting agent or an anti-infective agent), applied to or infiltrated into cardiac surgery site (or to the surface of an implanted device or implant) for the prevention of adhesions during open or endoscopic heart surgery include: (a) sprayable collagen-containing formulations such as COSTASIS or CT3; (b) sprayable PEG-containing formulations such as COSEAL, ADHIBIT, FOCALSEAL or DURASEAL; (c) fibrinogen-containing formulations such as FLOSEAL or TISSEAL; (d) hyaluronic acid-containing formulations such as RESTYLANE or PERLANE, HYLAFORM, or SYNVISC; (e) polymeric gels for surgical implantation such as REPEL or FLOWGEL; (f) surgical adhesives containing cyanoacrylates such as DERMABOND, INDERMIL, GLUSTITCH, TISSUMEND, VETBOND, HISTOACRYL BLUE and ORABASE SOOTHE-N-SEAL LIQUID PROTECTANT; (g) dextran sulfate gels such as the ADCON series of gels; and (h) lipid based compositions such as ADSURF. It should be obvious to one of skill in the art that commercial compositions not specifically cited above as well as next-generation and/or subsequently-developed commercial products are to be anticipated and are suitable for use under the present invention.

Virtually any manipulation of the chest wall, pericardium and heart can trigger a cascade that ultimately results in the formation of adhesions. In many instances, the adhesions must be broken down during repeat open-heart interventions. An adhesion barrier, either alone or containing a fibrosis-inhibiting agent (and/or an anti-infective agent), is best applied directly to the affected areas (as a solid, a film, a paste, a gel, a liquid or another such formulation) during open or endoscopic cardiac procedures. In a preferred embodiment, the barrier (alone or containing an anti-fibrotic and/or anti-infective agent) is sprayed under direct or endoscopic vision during the procedure onto the heart, pericardium, pleura and chest wall that are operated on, or manipulated, during the intervention. Since adhesions often occur in areas at a distance from the tissues actually instrumented during a surgical intervention, it is recommended that the barrier (with or without a therapeutic agent) be applied to a wide area in the chest (potentially even the entire cardiopulmonary viscera and infiltrated throughout the pericardial sac). Preferred barriers include films, liquids, gels, pastes, sprays or other formulations that can be delivered during open procedures or through an endoscope, adhere to the tissues treated, and remain in place long enough to deliver the therapeutic agent and/or prevent adhesion formation. As an alternative, the therapeutic agent can be delivered directly into the pericardial sac as an injectable (either before, during or after the procedure) such that the drug is delivered in doses high enough and long enough (multiple dosing and/or sustained release preparations are preferred) to prevent adhesions and the complications arising from them. An ideal adhesion therapy will reduce the incidence, number and tenacity of adhesions and improve patient outcome by reducing the complications of repeat interventions.

As described above, the compositions for the prevention of surgical adhesions can be applied directly or indirectly to the tissue in a cardiac surgery procedure. The polymeric compositions (either with or without an anti-fibrotic or anti-infective therapeutic agent) can be administered in any manner described herein. Exemplary methods include either direct application at the time of surgery or with endoscopic, ultrasound, CT, MRI, or fluoroscopic guidance. If an implanted device is being placed, the composition for the prevention of adhesions can be applied to the surface of the implant, or to the surrounding tissues, in conjunction with placement of a medical device or implant at the surgical site. Representative examples of implants for use in cardiac procedures includes, without limitation, heart valves (porcine, artificial), ventricular assist devices, cardiac pumps, artificial hearts, stents, bypass grafts (artificial and endogenous), patches, cardiac electrical leads, defibrillators and pacemakers.

The polymeric composition, with or without a fibrosis-inhibiting agent, may be applied during open or endoscopic heart surgery: (a) to the tissue surface of the pericardium (or infiltrated into the pericardial sac), heart, great vessels, pleura, lungs, chest wall and any adjacent affected tissues (e.g., as an injectable, solution, paste, gel, in situ forming gel or mesh) during the surgical procedure; (b) to the surface of an implanted device or implant and/or the tissue surrounding the implant (e.g., as an injectable, solution, paste, gel, in situ forming gel or mesh) before, during, or after the surgical procedure; (c) by intraperitoneal or endoscopic injection of the composition into the anatomical space (i.e., the pericardial sac) at the surgical site (particularly useful for this embodiment is the use of injectable compositions containing polymeric carriers which release the fibrosis-inhibiting agent over a period ranging from several hours to several weeks—fluids, suspensions, emulsions, microemulsions, microspheres, pastes, gels, microparticulates, sprays, aerosols, solid implants and other formulations which release the agent and can be delivered into the region where there is a risk of adhesion formation); (d) via percutaneous injection into the tissue as a solution as an infusate or as a sustained release preparation (intrapericardial injection); (e) by guided catheter or endoscopic injection of the composition into the lumen or the walls of the atria, ventricles, great vessels, coronary arteries or the pericardial sac (particularly useful for this embodiment is the use of injectable compositions containing polymeric carriers which release the fibrosis-inhibiting agent over a period ranging from several hours to several weeks—fluids, suspensions, emulsions, microemulsions, microspheres, pastes, gels, microparticulates, sprays, aerosols, solid implants and other formulations which release the agent can be delivered into the areas of the heart where there is a risk of adhesion formation); and/or (f) by any combination of the aforementioned methods. Combination therapies (i.e., combinations of therapeutic agents and combinations with antithrombotic, anti-infective, and/or antiplatelet agents) can also be used in the manner described above.

In certain applications involving the placement of a cardiac medical—device or implant, it may be desirable to apply the anti-fibrosis (and/or anti-infective) composition at a site that is adjacent to an implant (preferably near the implant-tissue interface). This can be accomplished during open, endoscopic or catheter-based procedures by applying the polymeric composition, with or without a fibrosis-inhibiting agent: (a) to the implant surface (e.g., as an injectable, solution, paste, gel, in situ forming gel, or mesh) before, during, or after the implantation procedure; (b) to the surface of the adjacent tissue (e.g., as an injectable, solution, paste, gel, in situ forming gel, or mesh) immediately prior to, during, or after implantation of the implant; (c) to the surface of the implant and the tissue surrounding the implant (e.g., as an injectable, solution, paste, gel, in situ forming gel or mesh) before, during, or after implantation of the implant; (d) by topical application of the composition into the anatomical space (pericardial sac, intracardiac, intra-arterial) where the implant will be placed (particularly useful for this embodiment is the use of polymeric carriers which release the fibrosis-inhibiting agent over a period ranging from several hours to several weeks—fluids, suspensions, emulsions, microemulsions, microspheres, pastes, gels, microparticulates, sprays, aerosols, solid implants and other formulations which release the agent can be delivered into the region where the device will be inserted); (e) via percutaneous injection into the tissue surrounding the implant as a solution, as an infusate, or as a sustained release preparation; and/or (f) by any combination of the aforementioned methods. Combination therapies (i.e., combinations of therapeutic agents and combinations with antithrombotic, anti-infective, and/or antiplatelet agents) can also be used.

In one aspect, the polymeric composition may be delivered to the heart (or device/tissue interface) in the form of a spray or gel during open, endoscopic or catheter-based procedures. The fibrosis-inhibiting agent can be incorporated directly into the surgical adhesion barrier or it can be incorporated into a secondary carrier (polymeric or non-polymeric), as described above, that is then incorporated into the adhesion barrier. Examples of polymer compositions that may be in the form of a spray or gel include poly(ethylene glycol)-based systems, hyaluronic acid and crosslinked hyaluronic acid compositions. These compositions can be applied as the final composition or they can be applied as materials that form a crosslinked gel in situ.

In another aspect, an activated polymer is dissolved in a biologically acceptable buffer that has a pH lower that 6.8. The resultant solution is then applied to the desired tissue surface in the presence of a second biologically acceptable buffer that has a pH greater than 7.5. Application of the reaction mixture to the tissue site may be by extrusion, brushing, spraying or by any other convenient means. Following application of the composition to the surgical site, any excess solution may be removed from the surgical site if deemed necessary. At this point in time, the surgical site can be closed using conventional means (e.g., sutures, staples, or a bioadhesive). In one embodiment, the activated polymer can form a covalent bond with the tissue to which it is applied may be used. Polymers containing and/or terminated with electrophilic groups such as succinimidyl, aldehyde, epoxide, isocyanate, vinyl, vinyl sulfone, maleimide, —S—S—(C₅H₄N) or activated esters, such as are used in peptide synthesis may be used as the reagents. For example, a 4 armed NHS-derivatized polyethylene glycol (e.g., pentaerythritol poly(ethylene glycol)ether tetra-succinimidyl glutarate) may be applied to the tissue in the solid form or in a solution form. In this embodiment, the 4 armed NHS-derivatized polyethylene glycol is dissolved in an acidic solution (pH about 2-3) and is then co-applied to the tissue using a basic buffer (pH>about 8). The antifibrosisfibrosis-inhibiting agent(s) may be incorporated directly into either the 4 armed NHS-derivatized polyethylene glycol, the acidic solution or the basic buffer. In another embodiment, the fibrosis-inhibiting agent may be incorporated into a secondary carrier that may then be incorporated into the 4 armed NHS-derivatized polyethylene glycol, the acidic solution and/or the basic buffer. The secondary carriers may include microparticles and/or microspheres which are made from degradable polymers. The degradable polymers may include polyesters, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X—Y, Y—X—Y, R—(Y—X)ₙ, R—(X—Y)ₙ and X—Y—X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator. In another embodiment, the tissue reactive polymer may be applied initially and then the fibrosis-inhibiting agent may then be applied to the coated tissue. The fibrosis-inhibiting agent may be applied directly to the tissue or it may be incorporated into a secondary carrier. The secondary carriers may include microspheres (as described above), microparticles (as described above), gels (e.g., hyaluronic acid, carboxymethyl cellulose, dextran, poly(ethylene oxide)—poly(propylene oxide) block copolymers as well as blends, association complexes and crosslinked compositions thereof) and films (degradable polyesters, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X—Y, Y—X—Y, R—(Y—X)ₙ, R—(X—Y)ₙ and X—Y—X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator, hyaluronic acid, carboxymethyl cellulose, dextran, poly(ethylene oxide)—poly(propylene oxide) block copolymers as well as blends, association complexes and crosslinked compositions thereof.

In yet another aspect, an activated polymer can be applied to the surgical site in the solid state. The activated polymer can react with the tissue surface to which it was applied as the polymer hydrates. A biologically acceptable buffer, with a pH greater than 7.5 can be applied to the tissue before and/or after the solid activated polymer has been applied. In one embodiment, the activated polymer can form a covalent bond with the tissue to which it is applied may be used. Polymers containing and/or terminated with electrophilic groups such as succinimidyl, aldehyde, epoxide, isocyanate, vinyl, vinyl sulfone, maleimide, —S—S—(C₅H₄N) or activated esters, such as are used in peptide synthesis may be used as the reagents. For example, a 4 armed NHS-derivatized polyethylene glycol (e.g., pentaerythritol poly(ethylene glycol)ether tetra-succinimidyl glutarate) may be applied to the tissue in the solid form. The antifibrosisfibrosis-inhibiting agent(s) may be incorporated directly into either the 4 armed NHS-derivatized polyethylene glycol, or the basic buffer. In another embodiment, the fibrosis-inhibiting agent may be incorporated into a secondary carrier that may then be incorporated into the 4 armed NHS-derivatized polyethylene glycol, and/or the basic buffer. The secondary carriers may include microparticles and/or microspheres which are made from degradable polymers. The degradable polymers may include polyesters, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X—Y, Y—X—Y, R—(Y—X)ₙ, R—(X—Y)ₙ and X—Y—X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator. In another embodiment, the tissue reactive polymer may be applied initially and then the fibrosis-inhibiting agent may then be applied to the coated tissue. The fibrosis-inhibiting agent may be applied directly to the tissue or it may be incorporated into a secondary carrier. The secondary carriers may include microspheres (as described above), microparticles (as described above), gels (e.g., hyaluronic acid, carboxymethyl cellulose, dextran, poly(ethylene oxide)—poly(propylene oxide) block copolymers as well as blends, association complexes and crosslinked compositions thereof) and films (degradable polyesters, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X—Y, Y—X—Y, R—(Y—X)ₙ, R—(X—Y)ₙ and X—Y—X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator, hyaluronic acid, carboxymethyl cellulose, dextran, poly(ethylene oxide)—poly(propylene oxide) block copolymers as well as blends, association complexes and crosslinked compositions thereof.

v) Adhesion Prevention in Orthopedic Procedures

In one aspect, adhesions may be associated with an orthopedic surgical procedure. Many orthopedic surgical interventions are performed as a result of injury or trauma (fractures; torn ligaments, cartilage, tendons or muscles) that cause significant tissue damage that can lead to excessive scarring and adhesion formation. As a result, orthopedic procedures often result in potentially severe post-operative complications which may be attributed to the trauma which caused the injury or to the trauma from the surgery itself. In general, excessive scarring and adhesion formation in orthopedic conditions follows certain patterns: (a) in joint injuries, it can result in a deformity such that the joint cannot fully extend, flex, or rotate (contractures); (b) in tendon injuries, it can prevent normal movement and lead to shortening; (c) in cartilage injuries, it can lead to the conversion of hyaline cartilage to fibrocartilage with a resultant loss of function and joint instability; (d) in muscle injuries, it can cause adhesion to adjacent tissues, loss of strength and loss of function; (e) in nerve injuries, it can result in loss of conduction and function; if the nerve becomes entrapped (encircled and constricted) by scar, it can cause pain, sensory impairment and loss of motor function; and (f) in tendons and ligaments, it can cause shortening, loss of range of motion and impaired function. The complications of adhesions can be wide spread; for example, adhesions formed after spinal surgery may produce low back pain, leg pain and sphincter disturbance (bladder and bowel). For this reason strategies designed to reduce adhesion formation in musculoskeletal surgery is a significant clinical problem. The local administration of anti-adhesive compositions, alone or loaded with a fibrosis-inhibiting agent, can be utilized in a wide array of clinical situations and conditions to improve patient outcomes following emergency or elective orthopedic interventions.

As described previously, adhesion barriers are frequently used in the management of adhesions following orthopedic procedures. A variety of commercially available adhesion barriers are suitable for combining with a fibrosis-inhibitor (and/or an anti-infective agent) in the management of orthopedic surgery adhesions. Closure Medical Corp. (Raleigh, N.C.) is developing a cyanoacrylate-based internal adhesives that may be used to seal internal surgical incisions or grafts which may be compatible in orthopedic and general surgical specialties. Products that are being developed by Closure Medical, Corp. are described in, for example, U.S. Pat. Nos. 6,620,846; 6,579,469; 6,565,840; 6,547,467 and 5,981,621. Life Medical Sciences, Inc. is developing the products, REPEL, REPEL-CV, RESOLVE and RELIEVE that are in various stages of development and may be used to prevent surgical adhesions in orthopedic and spinal surgeries. Products being developed by Life Medical Sciences, Inc. are described in, for example, U.S. Pat. Nos. 6,696,499; 6,399,624; 6,211,249; 6,136,333 and 5,711,958.

Other commercially available materials that may be used alone, or loaded with a therapeutic agent (e.g., a fibrosis-inhibiting agent or an anti-infective agent), applied to or infiltrated into an orthopedic site (or to the surface of an implanted device or implant) for the prevention of adhesions in open or endoscopic orthopedic surgery include: (a) sprayable collagen-containing formulations such as COSTASIS or CT3; (b) sprayable PEG-containing formulations such as COSEAL, ADHIBIT, FOCALSEAL, SPRAYGEL or DURASEAL; (c) fibrinogen-containing formulations such as FLOSEAL or TISSEAL; (d) hyaluronic acid-containing formulations such as RESTYLANE, HYLAFORM, PERLANE, SYNVISC, SEPRAFILM, SEPRACOAT, INTERGEL, or LUBRICOAT; (e) polymeric gels for surgical implantation such as REPEL or FLOWGEL; (f) orthopedic “cements” used to hold prostheses and tissues in place, such as OSTEOBOND (Zimmer), LVC (Wright Medical Technology), SIMPLEX P (Stryker), PALACOS (Smith & Nephew), and ENDURANCE (Johnson & Johnson, Inc.); (g) surgical adhesives containing cyanoacrylates such as DERMABOND, INDERMIL, GLUSTITCH, TISSUMEND, VETBOND, HISTOACRYL BLUE and ORABASE SOOTHE-N-SEAL LIQUID PROTECTANT; (g) implants containing hydroxyapatite (or synthetic bone material such as calcium sulfate, VITOSS (Orthovita) and CORTOSS (Orthovita)); (h) other biocompatible tissue fillers, such as those made by BioCure, 3M Company and Neomend; (i) polysacharride gels such as the ADCON series of gels; (j) films, sponges or meshes such as INTERCEED, VICRYL mesh, and GELFOAM; (o) lipid based compositions such as ADSURF; and (p) OSSIGEL, a viscous formulation of hyaluronic acid (HA) and basic fibroblast growth factor (bFGF) designed to accelerate bone fracture healing (Orquest, Inc.). It should be obvious to one of skill in the art that commercial compositions not specifically cited above as well as next-generation and/or subsequently-developed commercial products are to be anticipated and are suitable for use under the present invention.

Orthopedic surgical procedures are performed for a variety of conditions including fractures (open and closed), sprains, joint dislocations, crush injuries, ligament and muscle tears, tendon injuries, nerve injuries, congenital deformities and malformations, total joint or partial joint replacement, and cartilage injuries. Although many procedures are still performed through open surgical techniques, increasingly, numerous orthopedic procedures are being performed via an arthroscope inserted into the joint. Virtually any musculoskeletal (muscle, tendon, joint, bone, cartilage) injury, traumatic injury, or orthopedic surgical intervention can trigger a cascade that ultimately results in the formation of adhesions. In many instances, the adhesions must be broken down during repeat surgical interventions (e.g., capsulotomies, tendon releases, nerve entrapment releases, frozen joints, etc.). An adhesion barrier, either alone or containing a fibrosis-inhibiting agent (and/or an anti-infective agent), is best applied directly to the affected areas (as a solid, a film, a paste, a gel, a liquid or another such formulation) during open or arthroscopic orthopedic procedures. In a preferred embodiment, the barrier (alone or containing an anti-fibrotic and/or anti-infective agent) is sprayed under direct or arthrocopic vision onto the affected musculoskeletal tissue during the intervention. Since adhesions often occur in areas at a distance from the tissues actually instrumented during a surgical intervention, it is recommended that the barrier (with or without a therapeutic agent) be applied to a wide area around the injured or repaired tissues. Preferred barriers include films, liquids, gels, pastes, sprays or other formulations that can be delivered during open procedures or through an endoscope, adhere to the tissues treated, and remain in place long enough to deliver the therapeutic agent and/or prevent adhesion formation. An ideal adhesion therapy will reduce the incidence, number and tenacity of adhesions and improve patient outcome by reducing pain, weakness and sensory abnormalities, preventing contractures, increasing range of motion, improving function, limiting physical deformity and disability, and reducing the need for repeat interventions.

As described above, the compositions for the prevention of surgical adhesions can be applied directly or indirectly to the tissue in an orthopedic surgery procedure. The polymeric compositions (either with or without an anti-fibrotic or anti-infective therapeutic agent) can be administered in any manner described herein. Exemplary methods include either direct application at the time of surgery or with arthroscopic, ultrasound, CT, MRI, or fluoroscopic guidance. If an implanted device is being placed, the composition for the prevention of adhesions can be applied to the surface of the implant, or to the surrounding tissues, in conjunction with placement of a medical device or implant at the surgical site. Representative examples of implants for use in orthopedic procedures include plates, rods, screws, pins, wires, total and partial joint prostheses (artificial hips, knees, shoulders, phalangeal joints), reinforcement patches, tissue fillers, synthetic bone fillers, bone cement, synthetic graft material, allograft material, autograft material, artificial discs, spinal cages, and intermedulary rods.

The polymeric composition, with or without a fibrosis-inhibiting agent, may be applied during open or arthroscopic orthopedic surgery: (a) to the tissue surface of the bone, joint, muscle, tendon, ligament, cartilage and any adjacent affected tissues (e.g., as an injectable, solution, paste, gel, in situ forming gel or mesh) during the surgical procedure; (b) to the surface of an implanted orthopedic device or implant and/or the tissue surrounding the implant (e.g., as an injectable, solution, paste, gel, in situ forming gel or mesh) before, during, or after the surgical procedure; (c) by intra-articular or endoscopic administration of the composition into the anatomical space (e.g., the joint space, tendon sheath, nerve root, spinal canal) at the surgical site (particularly useful for this embodiment is the use of injectable compositions containing polymeric carriers which release the fibrosis-inhibiting agent over a period ranging from several hours to several weeks—fluids, suspensions, emulsions, microemulsions, microspheres, pastes, gels, microparticulates, sprays, aerosols, solid implants and other formulations which release the agent and can be delivered into the region where there is a risk of adhesion formation); (d) via percutaneous injection into the tissue as a solution as an infusate or as a sustained release preparation (intramuscular or intra-articular injection); (e) by guided catheter injection of the composition into the tissues and/or (f) by any combination of the aforementioned methods. Combination therapies (i.e., combinations of therapeutic agents and combinations with antithrombotic, anti-infective, and/or antiplatelet agents) can also be used in the manner described above.

In certain applications involving the placement of an orthopedic medical device or implant, it may be desirable to apply the anti-fibrosis (or anti-infective) composition at a site that is adjacent to an implant (preferably near the implant-tissue interface). This can be accomplished during open, endoscopic or catheter-based orthopedic procedures by applying the polymeric composition, with or without a fibrosis-inhibiting agent: (a) to the implant surface (e.g., as an injectable, solution, paste, gel, in situ forming gel, or mesh) before, during, or after the implantation procedure; (b) to the surface of the adjacent tissue (e.g., as an injectable, solution, paste, gel, in situ forming gel, or mesh) immediately prior to, during, or after implantation of the orthopedic implant; (c) to the surface of the implant and the tissue surrounding the implant (e.g., as an injectable, solution, paste, gel, in situ forming gel or mesh) before, during, or after implantation of the implant; (d) by topical application of the composition into the anatomical space (joint capsule, spinal canal, marrow, tendon sheath etc.) where the implant will be placed (particularly useful for this embodiment is the use of polymeric carriers which release the fibrosis-inhibiting agent over a period ranging from several hours to several weeks—fluids, suspensions, emulsions, microemulsions, microspheres, pastes, gels, microparticulates, sprays, aerosols, solid implants and other formulations which release the agent can be delivered into the region where the device will be inserted); (e) via percutaneous injection into the tissue surrounding the orthopedic implant as a solution, as an infusate, or as a sustained release preparation; and/or (f) by any combination of the aforementioned methods. Combination therapies (i.e., combinations of therapeutic agents and combinations with antithrombotic, anti-infective, and/or antiplatelet agents) can also be used.

In one aspect, the polymeric composition may be delivered to the musculoskeletal tissue (or device/tissue interface) in the form of a spray or gel during open, endoscopic or catheter-based procedures. The fibrosis-inhibiting (and/or anti-infective) agent can be incorporated directly into the surgical adhesion barrier or it can be incorporated into a secondary carrier (polymeric or non-polymeric), as described above, that is then incorporated into the adhesion barrier. Examples of polymer compositions that may be in the form of a spray or gel include poly(ethylene glycol)-based systems, hyaluronic acid and crosslinked hyaluronic acid compositions. These compositions can be applied as the final composition or they can be applied as materials that form a crosslinked gel in situ.

In another aspect, an activated polymer is dissolved in a biologically acceptable buffer that has a pH lower that 6.8. The resultant solution is then applied to the desired tissue surface in the presence of a second biologically acceptable buffer that has a pH greater than 7.5. Application of the reaction mixture to the tissue site may be by extrusion, brushing, spraying or by any other convenient means. Following application of the composition to the surgical site, any excess solution may be removed from the surgical site if deemed necessary. At this point in time, the surgical site can be closed using conventional means (e.g., sutures, staples, or a bioadhesive). In one embodiment, the activated polymer can form a covalent bond with the tissue to which it is applied may be used. Polymers containing and/or terminated with electrophilic groups such as succinimidyl, aldehyde, epoxide, isocyanate, vinyl, vinyl sulfone, maleimide, —S—S—(C₅H₄N) or activated esters, such as are used in peptide synthesis may be used as the reagents. For example, a 4 armed NHS-derivatized polyethylene glycol (e.g., pentaerythritol poly(ethylene glycol)ether tetra-succinimidyl glutarate) may be applied to the tissue in the solid form or in a solution form. In this embodiment, the 4 armed NHS-derivatized polyethylene glycol is dissolved in an acidic solution (pH about 2-3) and is then co-applied to the tissue using a basic buffer (pH>about 8). The antifibrosisfibrosis-inhibiting agent(s) may be incorporated directly into either the 4 armed NHS-derivatized polyethylene glycol, the acidic solution or the basic buffer. In another embodiment, the fibrosis-inhibiting agent may be incorporated into a secondary carrier that may then be incorporated into the 4 armed NHS-derivatized polyethylene glycol, the acidic solution and/or the basic buffer. The secondary carriers may include microparticles and/or microspheres which are made from degradable polymers. The degradable polymers may include polyesters, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X—Y, Y—X—Y, R—(Y—X)ₙ, R—(X—Y)ₙ and X—Y—X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator. In another embodiment, the tissue reactive polymer may be applied initially and then the fibrosis-inhibiting agent may then be applied to the coated tissue. The fibrosis-inhibiting agent may be applied directly to the tissue or it may be incorporated into a secondary carrier. The secondary carriers may include microspheres (as described above), microparticles (as described above), gels (e.g., hyaluronic acid, carboxymethyl cellulose, dextran, poly(ethylene oxide)—poly(propylene oxide) block copolymers as well as blends, association complexes and crosslinked compositions thereof) and films (degradable polyesters, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X—Y, Y—X—Y, R—(Y—X)ₙ, R—(X—Y)ₙ and X—Y—X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator, hyaluronic acid, carboxymethyl cellulose, dextran, poly(ethylene oxide)—poly(propylene oxide) block copolymers as well as blends, association complexes and crosslinked compositions thereof.

In yet another aspect, an activated polymer can be applied to the surgical site in the solid state. The activated polymer can react with the tissue surface to which it was applied as the polymer hydrates. A biologically acceptable buffer, with a pH greater than 7.5 can be applied to the tissue before and/or after the solid activated polymer has been applied. In one embodiment, the activated polymer can form a covalent bond with the tissue to which it is applied may be used. Polymers containing and/or terminated with electrophilic groups such as succinimidyl, aldehyde, epoxide, isocyanate, vinyl, vinyl sulfone, maleimide, —S—S—(C₅H₄N) or activated esters, such as are used in peptide synthesis may be used as the reagents. For example, a 4 armed NHS-derivatized polyethylene glycol (e.g., pentaerythritol poly(ethylene glycol)ether tetra-succinimidyl glutarate) may be applied to the tissue in the solid form. The antifibrosisfibrosis-inhibiting agent(s) may be incorporated directly into either the 4 armed NHS-derivatized polyethylene glycol, or the basic buffer. In another embodiment, the fibrosis-inhibiting agent may be incorporated into a secondary carrier that may then be incorporated into the 4 armed NHS-derivatized polyethylene glycol, and/or the basic buffer. The secondary carriers may include microparticles and/or microspheres which are made from degradable polymers. The degradable polymers may include polyesters, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X—Y, Y—X—Y, R—(Y—X)ₙ, R—(X—Y)ₙ, and X—Y—X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator. In another embodiment, the tissue reactive polymer may be applied initially and then the fibrosis-inhibiting agent may then be applied to the coated tissue. The fibrosis-inhibiting agent may be applied directly to the tissue or it may be incorporated into a secondary carrier. The secondary carriers may include microspheres (as described above), microparticles (as described above), gels (e.g., hyaluronic acid, carboxymethyl cellulose, dextran, poly(ethylene oxide)—poly(propylene oxide) block copolymers as well as blends, association complexes and crosslinked compositions thereof) and films (degradable polyesters, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X—Y, Y—X—Y, R—(Y—X)ₙ, R—(X—Y)ₙ and X—Y—X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator, hyaluronic acid, carboxymethyl cellulose, dextran, poly(ethylene oxide)—poly(propylene oxide) block copolymers as well as blends, association complexes and crosslinked compositions thereof.

vi) Adhesion Prevention in Reconstructive and Cosmetic Procedures

In one aspect, adhesions may be associated with a cosmetic or reconstructive surgical procedure. The use of soft tissue implants for cosmetic applications (aesthetic and reconstructive) is common in breast augmentation, breast reconstruction after cancer surgery, craniofacial procedures, reconstruction after trauma, congenital craniofacial reconstruction and oculoplastic surgical procedures to name a few.

The clinical function of a soft tissue implant depends upon the implant being able to effectively maintain its shape over time. In many instances, when these devices are implanted in the body, they are subject to a “foreign body” response from the surrounding host tissues. The body recognizes the implanted device as foreign, which triggers an inflammatory response followed by encapsulation of the implant with fibrous connective tissue (adhesion formation). Encapsulation of surgical implants complicates a variety of reconstructive and cosmetic surgeries, but is particularly problematic in the case of breast reconstruction surgery where the breast implant becomes surrounded by a fibrous capsule that alters anatomy and function. Scar capsules that harden and contract (known as “capsular contractures”) are the most common complication of breast implant or reconstructive surgery. Capsular (fibrous) contractures can result in hardening of the breast, loss of the normal anatomy and contour of the breast, discomfort, weakening and rupture of the implant shell, asymmetry, infection, and patient dissatisfaction. Further, fibrous encapsulation of any soft tissue implant can occur even after a successful implantation if the device is manipulated or irritated by the daily activities of the patient. Bleeding in and around the implant can also trigger a biological cascade that ultimately leads to excess scar tissue formation. Furthermore, certain types of implantable prostheses (such as breast implants) include gel fillers (e.g., silicone) that tend to leak through the membrane envelope of the implant and can potentially cause a chronic inflammatory response in the surrounding tissue (which encourages tissue encapsulation and contracture formation). The effects of unwanted scarring in the vicinity of the implant are the leading cause of additional surgeries to correct defects, break down scar tissue (capsulotomy or capsulaectomy), to replace the implant, or remove the implant. The local administration of anti-adhesive compositions, alone or loaded with a fibrosis-inhibiting agent, can be utilized in a wide array of cosmetic and reconstructive procedures to improve patient outcomes.

Soft tissue implants are used in a variety of cosmetic, plastic, and reconstructive surgical procedures and may be delivered to many different parts of the body, including, without limitation, the face, nose, breast, chin, buttocks, chest, lip and cheek. Soft tissue implants are used for the reconstruction of surgically or traumatically created tissue voids, augmentation of tissues or organs, contouring of tissues, the restoration of bulk to aging tissues, and to correct soft tissue folds or wrinkles (rhytides). Of all soft tissue implantation procedures, breast implant placement for augmentation or breast reconstruction after mastectomy is the most frequently performed cosmetic surgery implant procedure. For example, in 2002 alone, over 300,000 women had breast implant surgery. Of these, approximately 80,000 were breast reconstructions following a mastectomy due to cancer.

The process for failure of all soft tissue implants is similar regardless of anatomical placement. However, since breast implants have been the most widely studied soft tissue implant, they will be used to illustrate the present invention. In general, breast augmentation or reconstructive surgery involves the placement of a commercially available breast implant, consisting of a capsule filled with either saline or silicone, into the tissues underneath the mammary gland. Four different incision sites have historically been used for breast implantation: axillary (armpit), periareolar (around the underside of the nipple), inframamary (at the base of the breast where it meets the chest wall) and transumbilical (around the belly button). The tissue is dissected away through the small incision, often with the aid of an endoscope (particularly for axillary and transumbilical procedures where tunneling from the incision site to the breast is required). A pocket for placement of the breast implant is created in either the subglandular or the subpectorial region. For subglandular implants, the tissue is dissected to create a space between the glandular tissue and the pectoralis major muscle that extends down to the inframammary crease. For subpectoral implants, the fibers of the pectoralis major muscle are carefully dissected to create a space beneath the pectoralis major muscle and superficial to the rib cage. Careful hemostasis is essential (since it can contribute to complications such as capsular contractures), so much so that minimally invasive procedures (axillary, transumbilical approaches) must be converted to more open procedures (such as periareolar) if bleeding control is inadequate. Depending upon the type of surgical approach selected, the breast implant is often deflated and rolled up for placement in the patient. After accurate positioning is achieved, the implant can then be filled or expanded to the desired size.

Although many patients are satisfied with the initial procedure, significant percentages suffer from complications that frequently require a repeat intervention to correct. Encapsulation of a breast prosthesis that creates a periprosthetic shell (called capsular contracture) is the most common complication reported after breast enlargement, with up to 50% of patients reporting some dissatisfaction. Calcification can occur within the fibrous capsule adding to its firmness and complicating the interpretation of mammograms. Multiple causes of capsular contracture have identified including: foreign body reaction, migration of silicone gel molecules across the capsule and into the tissue, autoimmune disorders, genetic predisposition, infection, hematoma, and the surface characteristics of the prosthesis. Although no specific etiology has been repeatedly identified, at the cellular level, abnormal fibroblast activity stimulated by a foreign body is a consistent finding. Periprosthetic capsular tissues contain macrophages and occasional T- and B-lymphocytes, suggesting an inflammatory component to the process. Implant surfaces have been made both smooth and textured in an attempt to reduce encapsulation, however, neither has been proven to produce consistently superior results. Animal models suggest that there is an increased tendency for increased capsular thickness and contracture with textured surfaces that encourage fibrous tissue ingrowth on the surface. Placement of the implant in the subpectoral location appears to decrease the rate of encapsulation in both smooth and textured implants.

From a patient's perspective, the biological processes described above lead to a series of commonly described complaints. Implant malposition, hardness and unfavorable shape are the most frequently sited complications and are most often attributed to capsular contracture. When the surrounding scar capsule begins to harden and contract, it results in discomfort, weakening of the shell, asymmetry, skin dimpling and malpositioning. True capsular contractures will occur in approximately 10% of patients after augmentation, and in 25% to 30% of reconstruction cases, with most patients reporting dissatisfaction with the asthetic outcome. Scarring leading to asymmetries occurs in 10% of augmentations and 30% of reconstructions and is the leading cause of revision surgery. Skin wrinkling (due to the contracture pulling the skin in towards the implant) is a complication reported by 10% to 20% of patients. Scarring has even been implicated in implant deflation (1-6% of patients; saline leaking out of the implant and “deflating” it), when fibrous tissue ingrowth into the diaphragmatic valve (the access site used to inflate the implant) causes it to become incontinent and leak. In addition, over 15% of patients undergoing augmentation will suffer from chronic pain and many of these cases are ultimately attributable to scar tissue formation. Other complications of breast augmentation surgery include late leaks, hematoma (approximately 1-6% of patients), seroma (2.5%), hypertrophic scarring (2-5%) and infections (about 1-4% of cases).

Correction can involve several options including removal of the implant, capsulotomy (cutting or surgically releasing the capsule), capsulectomy (surgical removal of the fibrous capsule), or placing the implant in a different location (i.e., from subglandular to subpectoral). Ultimately, additional surgery (revisions, capsulotomy, removal, re-implantation) is required in over 20% of augmentation patients and in over 40% of reconstruction patients, with scar formation and capsular contracture being far and away the most common cause. Procedures to break down the scar may not be sufficient, and approximately 8% of augmentations and 25% of reconstructions ultimately have the implant surgically removed.

A fibrosis-inhibiting agent or composition delivered locally from the soft tissue implant or administered locally into the tissue surrounding the soft tissue implant can minimize fibrous tissue formation, encapsulation and capsular contracture. Application of a fibrosis-inhibiting composition onto the surface of a soft tissue implant or incorporated into a soft tissue implant (e.g., the agent is incorporated into the saline, gel or silicone within the implant and passively diffuses across the capsule into the surrounding tissue) may minimize or prevent fibrous contracture. Infiltration of a fibrosis-inhibiting agent or composition into the tissue surrounding the soft tissue implant, or into the surgical pocket where the implant will be placed, is another strategy for preventing the formation of scar and capsular contracture in augmentation and reconstructive surgery.

As described previously, adhesions and fibrous encapsulation of cosmetic implants is a common complication of asthetic and reconstructive surgery. A variety of commercially available adhesion barriers are suitable for combining with a fibrosis-inhibitor (and/or an anti-infective agent) in the management of this complication. Commercially available materials that may be used alone or loaded with a therapeutic agent (e.g., a fibrosis-inhibiting agent or an anti-infective agent), applied to the surface of a soft tissue implant, contained within the “filler” (typically saline, silicone or gel) of a soft tissue implant, or infiltrated into the tissue surrounding the implantation site for the prevention of adhesions in cosmetic surgery include: (a) sprayable collagen-containing formulations such as COSTASIS or CT3; (b) sprayable PEG-containing formulations such as COSEAL, ADHIBIT, FOCALSEAL, SPRAYGEL or DURASEAL; (c) fibrinogen-containing formulations such as FLOSEAL or TISSEAL; (d) hyaluronic acid-containing formulations such as RESTYLANE or PERLANE, HYLAFORM, SYNVISC, SEPRAFILM or SEPRACOAT; (e) polymeric gels for surgical implantation such as REPEL or FLOWGEL; (f) surgical adhesives containing cyanoacrylates such as DERMABOND, INDERMIL, GLUSTITCH, TISSUMEND, VETBOND, HISTOACRYL BLUE and ORABASE SOOTHE-N-SEAL LIQUID PROTECTANT; (g) dextran sulfate gels such as the ADCON series of gels; and (h) lipid based compositions such as ADSURF. Several of the above agents (e.g., formulations containing PEG, collagen, or fibrinogen such as COSEAL, CT3, ADHIBIT, COSTASIS, FOCALSEAL, SPRAYGEL, DURASEAL, TISSEAL AND FLOSEAL) have the added benefit of being hemostats and vascular sealants, which given the suspected role of inadequate hemostasis in the development of capsular contracture, should also be of benefit in the practice of this invention. It should be obvious to one of skill in the art that commercial compositions not specifically cited above as well as next-generation and/or subsequently-developed commercial products are to be anticipated and are suitable for use under the present invention.

As described above, the compositions for the prevention of surgical adhesions can be applied directly or indirectly to the tissue around the cosmetic implant site. The polymeric compositions (either with or without a therapeutic agent) can be administered in any manner described herein. Exemplary methods include either direct application at the time of surgery or with endoscopic, ultrasound, CT, MRI, or fluoroscopic guidance and in conjunction with placement of a cosmetic implant at the surgical site. Representative examples of implants for use in cosmetic procedures include, without limitation, saline breast implants, silicone breast implants, chin and mandibular implants, nasal implants, cheek implants, lip implants, other facial implants, pectoral and chest implants, malar and submalar implants, tissue fillers, and buttocks implants.

The polymeric composition, with or without a fibrosis-inhibiting agent, may be applied during open or endoscopic cosmetic surgery: (a) to the soft tissue implant surface (e.g., as an injectable, solution, paste, gel, in situ forming gel, or mesh) before, during, or after the implantation procedure; (b) to the surface of the tissue (e.g., as an injectable, solution, paste, gel, in situ forming gel or mesh) of the implantation pocket immediately prior to, or during implantation of the soft tissue implant; (c) to the surface of the soft tissue implant and/or the tissue surrounding the implant (e.g., as an injectable, solution, paste, gel, in situ forming gel or mesh) before, during, or after implantation of the soft tissue implant; (d) by topical application of the anti-fibrosis agent into the anatomical space where the soft tissue implant will be placed (particularly useful for this embodiment is the use of polymeric carriers which release the fibrosis-inhibiting agent over a period ranging from several hours to several weeks—fluids, suspensions, emulsions, microemulsions, microspheres, pastes, gels, microparticulates, sprays, aerosols, solid implants and other formulations which release the agent and can be delivered into the region where the implant will be inserted); (e) via percutaneous injection into the tissue surrounding the implant as a solution, as an infusate, or as a sustained release preparation; and/or (f) by any combination of the aforementioned methods. Combination therapies (i.e., combinations of therapeutic agents and combinations with antithrombotic, anti-infective, and/or antiplatelet agents) can also be used.

A composition that includes an anti-scarring agent can be infiltrated into the space (surgically created pocket) where the soft tissue implant will be implanted. In certain applications involving the placement of a cosmetic soft tissue implant, it may be desirable to apply the anti-fibrosis (or anti-infective) composition at a site that is adjacent to an implant (preferably near the implant-tissue interface). This can be accomplished during open, endoscopic or catheter-based cosmetic procedures by applying the polymeric composition, with or without a fibrosis-inhibiting agent: (a) to the implant surface (e.g., as an injectable, solution, paste, gel, in situ forming gel, or mesh) before, during, or after the implantation procedure; (b) to the surface of the adjacent tissue (e.g., as an injectable, solution, paste, gel, in situ forming gel, or mesh) immediately prior to, during, or after implantation of the soft tissue implant; (c) to the surface of the soft tissue implant and the tissue surrounding the implant (e.g., as an injectable, solution, paste, gel, in situ forming gel or mesh) before, during, or after implantation of the implant; (d) by topical application of the composition into the anatomical space (surgical pocket; for example, in breast implants this is the subglandular or subpectoral space) where the soft tissue implant will be placed (particularly useful for this embodiment is the use of polymeric carriers which release the fibrosis-inhibiting agent over a period ranging from several hours to several weeks—fluids, suspensions, emulsions, microemulsions, microspheres, pastes, gels, microparticulates, sprays, aerosols, solid implants and other formulations which release the agent can be delivered into the region where the device will be inserted); (e) via percutaneous injection into the tissue surrounding the soft tissue implant as a solution, as an infusate, or as a sustained release preparation; and/or (f) by any combination of the aforementioned methods. Combination therapies (i.e., combinations of therapeutic agents and combinations with antithrombotic, anti-infective, and/or antiplatelet agents) can also be used.

In one aspect, the polymeric composition may be delivered to the soft tissue implant (or implant/tissue interface) in the form of a spray or gel during open, endoscopic or catheter-based procedures. The fibrosis-inhibiting (and/or anti-infective) agent can be incorporated directly into the surgical adhesion barrier or it can be incorporated into a secondary carrier (polymeric or non-polymeric), as described above, that is then incorporated into the adhesion barrier. Examples of polymer compositions that may be in the form of a spray or gel include poly(ethylene glycol)-based systems, fibrinogen-containing systems, hyaluronic acid and crosslinked hyaluronic acid compositions. These compositions can be applied as the final composition or they can be applied as materials that form a crosslinked gel in situ.

In another aspect, an activated polymer is dissolved in a biologically acceptable buffer that has a pH lower that 6.8. The resultant solution is then applied to the desired tissue surface in the presence of a second biologically acceptable buffer that has a pH greater than 7.5. Application of the reaction mixture to the tissue site may be by extrusion, brushing, spraying or by any other convenient means. Following application of the composition to the surgical site, any excess solution may be removed from the surgical site if deemed necessary. At this point in time, the surgical site can be closed using conventional means (e.g., sutures, staples, or a bioadhesive). In one embodiment, the activated polymer can form a covalent bond with the tissue to which it is applied may be used. Polymers containing and/or terminated with electrophilic groups such as succinimidyl, aldehyde, epoxide, isocyanate, vinyl, vinyl sulfone, maleimide, —S—S—(C₅H₄N) or activated esters, such as are used in peptide synthesis may be used as the reagents. For example, a 4 armed NHS-derivatized polyethylene glycol (e.g., pentaerythritol poly(ethylene glycol)ether tetra-succinimidyl glutarate) may be applied to the tissue in the solid form or in a solution form. In this embodiment, the 4 armed NHS-derivatized polyethylene glycol is dissolved in an acidic solution (pH about 2-3) and is then co-applied to the tissue using a basic buffer (pH>about 8). The antifibrosisfibrosis-inhibiting agent(s) may be incorporated directly into either the 4 armed NHS-derivatized polyethylene glycol, the acidic solution or the basic buffer. In another embodiment, the fibrosis-inhibiting agent may be incorporated into a secondary carrier that may then be incorporated into the 4 armed NHS-derivatized polyethylene glycol, the acidic solution and/or the basic buffer. The secondary carriers may include microparticles and/or microspheres which are made from degradable polymers. The degradable polymers may include polyesters, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X—Y, Y—X—Y, R—(Y—X)ₙ, R—(X—Y)ₙ and X—Y—X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator. In another embodiment, the tissue reactive polymer may be applied initially and then the fibrosis-inhibiting agent may then be applied to the coated tissue. The fibrosis-inhibiting agent may be applied directly to the tissue or it may be incorporated into a secondary carrier. The secondary carriers may include microspheres (as described above), microparticles (as described above), gels (e.g., hyaluronic acid, carboxymethyl cellulose, dextran, poly(ethylene oxide)—poly(propylene oxide) block copolymers as well as blends, association complexes and crosslinked compositions thereof) and films (degradable polyesters, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X—Y, Y—X—Y, R—(Y—X)ₙ, R—(X—Y)ₙ and X—Y—X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator, hyaluronic acid, carboxymethyl cellulose, dextran, poly(ethylene oxide)—poly(propylene oxide) block copolymers as well as blends, association complexes and crosslinked compositions thereof.

In yet another aspect, an activated polymer can be applied to the surgical site in the solid state. The activated polymer can react with the tissue surface to which it was applied as the polymer hydrates. A biologically acceptable buffer, with a pH greater than 7.5 can be applied to the tissue before and/or after the solid activated polymer has been applied. In one embodiment, the activated polymer can form a covalent bond with the tissue to which it is applied may be used. Polymers containing and/or terminated with electrophilic groups such as succinimidyl, aldehyde, epoxide, isocyanate, vinyl, vinyl sulfone, maleimide, —S—S—(C₅H₄N) or activated esters, such as are used in peptide synthesis may be used as the reagents. For example, a 4 armed NHS-derivatized polyethylene glycol (e.g., pentaerythritol poly(ethylene glycol)ether tetra-succinimidyl glutarate) may be applied to the tissue in the solid form. The antifibrosisfibrosis-inhibiting agent(s) may be incorporated directly into either the 4 armed NHS-derivatized polyethylene glycol, or the basic buffer. In another embodiment, the fibrosis-inhibiting agent may be incorporated into a secondary carrier that may then be incorporated into the 4 armed NHS-derivatized polyethylene glycol, and/or the basic buffer. The secondary carriers may include microparticles and/or microspheres which are made from degradable polymers. The degradable polymers may include polyesters, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X—Y, Y—X—Y, R—(Y—X)ₙ, R—(X—Y)ₙ and X—Y—X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator. In another embodiment, the tissue reactive polymer may be applied initially and then the fibrosis-inhibiting agent may then be applied to the coated tissue. The fibrosis-inhibiting agent may be applied directly to the tissue or it may be incorporated into a secondary carrier. The secondary carriers may include microspheres (as described above), microparticles (as described above), gels (e.g., hyaluronic acid, carboxymethyl cellulose, dextran, poly(ethylene oxide)—poly(propylene oxide) block copolymers as well as blends, association complexes and crosslinked compositions thereof) and films (degradable polyesters, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X—Y, Y—X—Y, R—(Y—X)ₙ, R—(X—Y)ₙ and X—Y—X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator, hyaluronic acid, carboxymethyl cellulose, dextran, poly(ethylene oxide)—poly(propylene oxide) block copolymers as well as blends, association complexes and crosslinked compositions thereof.

vii) Agents and Dosages of Fibrosis-inhibitors

In certain aspects of the invention, compositions are provided that can release a therapeutic agent able to reduce scarring (i.e., a fibrosis-inhibiting agent) at a surgical site. Within one embodiment of the invention, surgical adhesion barriers may include or be adapted to release an agent that inhibits one or more of the five general components of the process of fibrosis (or scarring), including: inflammatory response and inflammation, migration and proliferation of connective tissue cells (such as fibroblasts or smooth muscle cells), formation of new blood vessels (angiogenesis), deposition of extracellular matrix (ECM), and remodeling (maturation and organization of the fibrous tissue). By inhibiting one or more of the components of fibrosis (or scarring), the overgrowth of scar tissue may be inhibited or reduced.

Examples of fibrosis-inhibiting agents for use in surgical adhesion barriers include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for surgical adhesion prevention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days. In one aspect, the drug is released in effective concentrations for a period ranging from 1-90 days.

The exemplary anti-fibrosing agents, used alone or in combination, should be administered under the following dosing guidelines. The total amount (dose) of anti-scarring agent in the composition can be in the range of about 0.01 μg-10 μg, or 10 μg-10 mg, or 10 mg-250 mg, or 250 mg-1000 mg, or 1000 mg-2500 mg. The dose (amount) of anti-scarring agent per unit area of surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring agents that can be used in conjunction with compositions for treating or preventing surgical adhesions in accordance with the invention. (A) Cell cycle inhibitors including doxorubicin and mitoxantrone. Doxorubicin analogues and derivatives thereof: total dose not to exceed 25 mg (range of 0.1 μg to 25 mg); preferred 1 μg to 5 mg. Dose per unit area of 0.01 μg-100 μg per mm²; preferred dose of 0.1 μg/mm²-10 μg/mm². Minimum concentration of 10⁻⁸-10⁻⁴ M of doxorubicin is to be maintained on the implant or barrier surface. Mitoxantrone and analogues and derivatives thereof: total dose not to exceed 5 mg (range of 0.01 μg to 5 mg); preferred 0.1 μg to 1 mg. Dose per unit area of 0.01 μg-20 μg per mm²; preferred dose of 0.05 μg/mm²-3 μg/mm². Minimum concentration of 10⁻⁸-10⁻⁴ M of mitoxantrone is to be maintained on the implant or barrier suface. (B) Cell cycle inhibitors including paclitaxel and analogues and derivatives (e.g., docetaxel) thereof: total dose not to exceed 10 mg (range of 0.1 μg to 10 mg); preferred 1 μg to 3 mg. Dose per unit area of 0.1 μg-10 μg per mm²; preferred dose of 0.25 μg/mm²-5 μg/mm². Minimum concentration of 10⁻⁸-10⁻⁴ M of paclitaxel is to be maintained on the implant or barrier suface. (C) Cell cycle inhibitors such as podophyllotoxins (e.g., etoposide): total dose not to exceed 10 mg (range of 0.1 μg to 10 mg); preferred 1 μg to 3 mg. Dose per unit area of 0.1 μg-10 μg per mm²; preferred dose of 0.25 μg/mm²-5 μg/mm². Minimum concentration of 10⁻⁸-10⁻⁴ M of etoposide is to be maintained on the implant or barrier suface. (D) Immunomodulators including sirolimus and everolimus. Sirolimus (i.e., rapamycin, RAPAMUNE): total dose not to exceed 10 mg (range of 0.1 μg to 10 mg); preferred 10 μg to 1 mg. Dose per unit area of 0.1 μg-100 μg per mm²; preferred dose of 0.5 μg/mm²-10 μg/mm². Minimum concentration of 10⁻⁸-10⁻⁴ M of sirolimus is to be maintained on the implant or barrier suface. Everolimus and derivatives and analogues thereof: total dose should not exceed 10 mg (range of 0.1 μg to 10 mg); preferred 10 μg to 1 mg. Dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the implant or barrier suface. (E) Heat shock protein 90 antagonists (e.g., geldanamycin) and analogues and derivatives thereof: total dose not to exceed 20 mg (range of 0.1 μg to 20 mg); preferred 1 μg to 5 mg. Dose per unit area of 0.1 μg-10 μg per mm²; preferred dose of 0.25 μg/mm²-5 μg/mm². Minimum concentration of 10⁻⁸-10⁻⁴ M of geldanamycin is to be maintained on the implant or barrier suface. (F) HMGCoA reductase inhibitors (e.g., simvastatin) and analogues and derivatives thereof: total dose not to exceed 2000 mg (range of 10.0 μg to 2000 mg); preferred 10 μg to 300 mg. Dose per unit area of 1.0 μg-1000 μg per mm²; preferred dose of 2.5 μg/mm²-500 μg/mm². Minimum concentration of 10⁻⁸-10⁻³ M of simvastatin is to be maintained on the implant or barrier suface. (G) Inosine monophosphate dehydrogenase inhibitors (e.g., mycophenolic acid, 1-alpha-25 dihydroxy vitamin D₃) and analogues and derivatives thereof: total dose not to exceed 2000 mg (range of 10.0 μg to 2000 mg); preferred 10 μg to 300 mg. Dose per unit area of 1.0 μg-1000 μg per mm²; preferred dose of 2.5 μg/mm²-500 μg/mm². Minimum concentration of 10⁻⁸-10⁻³ M of mycophenolic acid is to be maintained on the implant or barrier suface. (H) NF kappa B inhibitors (e.g., Bay 11-7082) and analogues and derivatives thereof: total dose not to exceed 200 mg (range of 1.0 μg to 200 mg); preferred 1 μg to 50 mg. Dose per unit area of 1.0 μg-100 μg per mm²; preferred dose of 2.5 μg/mm²-50 μg/mm². Minimum concentration of 10⁻⁸-10⁻⁴ M of Bay 11-7082 is to be maintained on the implant or barrier suface. (I) Antimycotic agents (e.g., sulconizole) and analogues and derivatives thereof: total dose not to exceed 2000 mg (range of 10.0 μg to 2000 mg); preferred 10 μg to 300 mg. Dose per unit area of 1.0 μg-1000 μg per mm²; preferred dose of 2.5 μg/mm²-500 μg/mm². Minimum concentration of 10⁻⁸-10⁻³ M of sulconizole is to be maintained on the implant or barrier suface and (J) p38 MAP kinase inhibitors (e.g., SB202190) and analogues and derivatives thereof: total dose not to exceed 2000 mg (range of 10.0 μg to 2000 mg); preferred 10 μg to 300 mg. Dose per unit area of 1.0 μg-1000 μg per mm²; preferred dose of 2.5 μg/mm²-500 μg/mm². Minimum concentration of 10⁻⁸-10⁻³ M of SB202190 is to be maintained on the implant or barrier suface.

According to another aspect, any anti-infective agent described above may be used in combination with the present compositions for surgical adhesion prevention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ M to 10⁻⁷ M, or about 10⁻⁷ M to 10⁻⁶ M about 10⁻⁶ M to 10⁻⁵ M or about 10⁻⁵ M to 10⁻⁴ M of the agent is maintained on the tissue surface.

Inflammatory Arthritis

In one aspect, the present invention provides compositions for the treatment and prevention of inflammatory arthritis. The compositions of the present invention can comprise one or more polymeric carriers and an anti-scarring agent.

Inflammatory arthritis is a serious health problem in developed countries, particularly given the increasing number of aged individuals and includes a variety of conditions including, but not limited to, rheumatoid arthritis, systemic lupus erythematosus, systemic sclerosis (scleroderma), mixed connective tissue disease, Sjögren's syndrome, ankylosing spondylitis, Behcet's syndrome, sarcoidosis, and osteoarthritis—all of which feature inflamed and/or painful joints as a prominent symptom.

In one aspect, the present compositions may be used to treat or prevent osteoarthritis (OA). Osteoarthritis is a common, debilitating, costly, and currently incurable disease. The disease is characterized by abnormal functioning of chondrocytes and their terminal differentiation, leading ultimately to the initiation of OA and the breakdown of the cartilage matrix in the articular cartilage of affected joints. Age is the most powerful risk factor for OA, but major joint trauma, excessive weight, and repetitive joint use are also important risk factors for OA. The pattern of joint involvement in OA is also influenced by prior vocational or avocational overload.

OA can be of primary (idiopathic) and secondary types. Primary OA is most commonly related to age. Repetitive use of the joints, particularly the weight-bearing joints such as hips, knees, feet and back, irritates and inflames the joints and causes joint pain and swelling. Eventually, cartilage begins to degenerate by flaking or forming tiny crevasses. In advanced cases, there is a total loss of the cartilage cushion between the bones of the joints. Loss of the cartilage cushion causes friction between the bones, leading to pain and limitation of joint mobility. Inflammation of the cartilage can also stimulate new bone outgrowths (spurs) to form around the joints.

Secondary OA is pathologically indistinguishable from idiopathic OA but is attributable to another disease or condition. Conditions that can lead to secondary OA include obesity, repeated trauma (e.g., ligament tears, cartilage tears), surgery to the joint structures (ligament repairs, menisectomy, cartilage removal), abnormal joints at birth (congenital abnormalities), gout, diabetes, and other metabolic disorders.

In one aspect, the present compositions may be used to treat or prevent rheumatoid arthritis (RA). Rheumatoid arthritis is a multisystem chronic, relapsing, inflammatory disease of unknown cause. Although many organs can be affected, RA is basically a severe form of chronic synovitis that sometimes leads to destruction and ankylosis of affected joints (Robbins Pathological Basis of Disease, by R. S. Cotran, V. Kumar, and S. L. Robbins, W. B. Saunders Co., 1989). Pathologically the disease is characterized by a marked thickening of the synovial membrane which forms villous projections that extend into the joint space, multilayering of the synoviocyte lining (synoviocyte proliferation), infiltration of the synovial membrane with white blood cells (macrophages, lymphocytes, plasma cells, and lymphoid follicles; called an “inflammatory synovitis”), and deposition of fibrin with cellular necrosis within the synovium. The tissue formed as a result of this process is called pannus and eventually the pannus grows to fill the joint space. The pannus develops an extensive network of new blood vessels through the process of angiogenesis which is essential to the evolution of the synovitis. Digestive enzymes (matrix metalloproteinases such as collagenase and stromelysin) and other mediators of the inflammatory process (e.g., hydrogen peroxide, superoxides, lysosomal enzymes, and products of arachadonic acid metabolism) released from the cells of the pannus tissue break down the cartilage matrix and cause progressive destruction of the cartilage. The pannus invades the articular cartilage leading to erosions and fragmentation of the cartilage tissue. Eventually there is erosion of the subchondral bone with fibrous ankylosis and ultimately bony ankylosis, of the involved joint.

It is generally believed, but not conclusively proven, that RA is an autoimmune disease, and that many different arthrogenic stimuli activate the immune response in the immunogenetically susceptible host. Both exogenous infectious agents (Ebstein-Barr virus, rubella virus, cytomegalovirus, herpes virus, human T-cell lymphotropic virus, mycoplasma, and others) and endogenous proteins (collagen, proteoglycans, altered immunoglobulins) have been implicated as the causative agent which triggers an inappropriate host immune response. Regardless of the inciting agent, autoimmunity plays a role in the progression of the disease. In particular, the relevant antigen is ingested by antigen-presenting cells (macrophages or dendritic cells in the synovial membrane), processed, and presented to T lymphocytes. The T cells initiate a cellular immune response and stimulate the proliferation and differentiation of B lymphocytes into plasma cells. The end result is the production of an excessive, inappropriate immune response directed against the host tissues (e.g., antibodies directed against type 11 collagen, antibodies directed against the Fc portion of autologous IgG (called “Rheumatoid Factor”)). This further amplifies the immune response and hastens the destruction of the cartilage tissue. Once this cascade is initiated, numerous mediators of cartilage destruction are responsible for the progression of rheumatoid arthritis.

In rheumatoid arthritis, articular cartilage is destroyed when it is invaded by pannus tissue (which is composed of inflammatory cells, blood vessels, and connective tissue). Generally, chronic inflammation in itself is insufficient to result in damage to the joint surface, but a permanent deficit is created once fibrovascular tissue digests the cartilage tissue. The abnormal growth of blood vessels and pannus tissue may be inhibited by treatment with fibrosis-inhibiting compositions, or fibrosis-inhibiting agents. Incorporation of an anti-scarring agent into these compositions or other intra-articular formulations, can provide an approach that can reduce the rate of progression of the disease.

Thus, within one aspect of the present invention, methods are provided for treating or preventing inflammatory arthritis comprising the step of administering to a patient in need thereof a therapeutically effective amount of an anti-scarring agent or a composition comprising an anti-scarring agent. Inflammatory arthritis includes a variety of conditions including, but not limited to, rheumatoid arthritis, systemic lupus erythematosus, systemic sclerosis (scleroderma), mixed connective tissue disease, Sjögren's syndrome, ankylosing spondylitis, Behcet's syndrome, sarcoidosis, and osteoarthritis—all of which feature inflamed and/or painful joints as a prominent symptom.

An effective anti-scarring therapy for inflammatory arthritis will accomplish one or more of the following: (i) decrease the severity of symptoms (pain, swelling and tenderness of affected joints; morning stiffness, weakness, fatigue, anorexia, weight loss); (ii) decrease the severity of clinical signs of the disease (thickening of the joint capsule, synovial hypertrophy, joint effusion, soft tissue contractures, decreased range of motion, ankylosis and fixed joint deformity); (iii) decrease the extra-articular manifestations of the disease (rheumatic nodules, vasculitis, pulmonary nodules, interstitial fibrosis, pericarditis, episcleritis, iritis, Felty's syndrome, osteoporosis); (iv) increase the frequency and duration of disease remission/symptom-free periods; (v) prevent fixed impairment and disability; and/or (vi) prevent/attenuate chronic progression of the disease.

According to the present invention, any anti-scarring agent described above could be utilized in the practice of this invention. Within certain embodiments of the invention, the composition may release an agent that inhibits one or more of the general components of the process of fibrosis (or scarring) associated with inflammatory arthritis, including: (a) formation of new blood vessels (angiogenesis), (b) migration and/or proliferation of connective tissue cells (such as fibroblasts or synoviocytes), (c) destruction of the cartilage matrix by metalloproteinase activity, (d) inflammatory response by cytokines (such as IL-1, TNFα, FGF, VEGF). By inhibiting one or more of the components of fibrosis (or scarring), cartilage loss may be inhibited or reduced.

In one aspect, the composition includes an anti-scarring agent and a polymeric carrier suitable for application to treat inflammatory arthritis. Numerous polymeric and non-polymeric delivery systems and compositions containing an anti-scarring agent for use in the treatment of inflammatory arthritis have been described above. An anti-scarring agent may be administered systemically (orally, intravenously, or by intramuscular or subcutaneous injection) in the minimum dose to achieve the above mentioned results. For patients with only a small number of joints affected, or with disease more prominent in a limited number of joints, the anti-scarring agent can be directly injected into the affected joint (intra-articular injection) via percutaneous needle insertion into the joint capsule, or as part of an arthroscopic procedure performed on the joint. In a preferred embodiment, the intra-articular formulation containing a fibrosis-inhibitor is administered to a joint following an injury with a high probability of inducing subsequent arthritis (e.g., cruciate ligament tears in the knee, meniscal tears in the knee). The agent is administered for a period sufficient (either through sustained release preparations and/or repeated injections) to protect the cartilage from breakdown as a result of the injury (or the surgical procedure used to treat it).

The anti-scarring agent can be administered in any manner described herein. However, preferred methods of administration include intravenous, oral, subcutaneous injection, or intramuscular injection. A particularly preferred embodiment involves the administration of the fibrosis-inhibiting compound as an intra-articular injection (directly, via arthroscopic or radiologic guidance, or irrigated into the joint as part of an open surgical procedure). The anti-scarring agent can be administered as a chronic low dose therapy to prevent disease progression, prolong disease remission, or decrease symptoms in active disease. Alternatively, the therapeutic agent can be administered in higher doses as a “pulse” therapy to induce remission in acutely active disease; such as the acute inflammation that follows a traumatic joint injury (intra-articular fractures, ligament tears, meniscal tears, as described below). The minimum dose capable of achieving these endpoints can be used and can vary according to patient, severity of disease, formulation of the administered agent, potency and/or tolerability of the agent, clearance of the agent from the joint, and route of administration.

In one preferred embodiment, the fibrosis-inhibiting composition can be an intra-articular injectable hyaluronic acid-based composition. Hyaluronic acid, which is a normal element of joint synovial fluid, lubricates the joint surface during normal activities (resting, walking) and helps prevent mechanical damage and decrease shock on the joint in high impact activities (such as running, jumping). In patients with OA, the elasticity and viscosity of the synovial fluid and the synovial hyaluronic acid concentration are reduced. It is believed that this contributes to the breakdown of the articular cartilage within the joint. Intra-articularly administered HA (typically sodium hyaluronate) penetrates the articular cartilage surface, the synovial tissue, and the capsule of the joint for a period of time after injection. By injecting hyaluronic acid into the joint (known as visco-supplementation), it is possible to partially restore the normal environment of the synovial fluid, reduce pain, and potentially prevent further damage and disability. Representative examples of hyaluronic acid compositions used in visco-supplementation are described in U.S. Pat. Nos. 6,654,120, 6,645,945, and 6,635,287. As such, HA-containing materials are administered as an intra-articular injection (as either a single treatment or a course of repeated treatment cycles) for the treatment of painful osteoarthritis of the knee in patients who have insufficient pain relief from conservative therapies. Occasionally other joints such as hips (injected under fluoroscopy), ankles, shoulders and elbow joints, are also injected with HA to relieve the symptoms of the disease in those particular joints. Depending upon the particular commercial product, the HA material is injected into the joint once a week for 5 to 6 consecutive weeks. When effective, patients may report that they receive symptomatic relief for a period of 6 months or more—at which time the cycle may be repeated to prolong the activity of the therapy. Despite the sustained benefit in some patients, the injected HA is rapidly cleared (removed) from the joint by the body over a period of several days. Prolonging the residence time of the HA in the joint by inhibiting its breakdown may be expected to enhance its efficacy and increase the duration of symptomatic relief. By adding a fibrosis-inhibiting agent to the HA, the intra-articular injection has the added benefit of helping to prevent cartilage breakdown (i.e., it is “chondroprotective”).

A variety of commercially available HA compositions for the treatment of inflammatory arthritis may be combined with one or more agents according to the present invention including: SYNVISC (Biomatrix, Inc., Ridgefield, N.J.)—an elastoviscous fluid containing hylan (a derivative of sodium hyaluronate (hyaluronan)) polymers derived from rooster combs, HYALGAN (Sanofi-Synthelabo Inc. New York, N.Y.), and ORTHOVISC (Ortho Biotech Products, Bridgewater, N.J.)—a highly purified, high molecular weight, high viscosity injectable form of HA intended to relieve pain and to improve joint mobility and range of motion in patients suffering from osteoarthritis (OA) of the knee. ORTHOVISC is injected into the knee to restore the elasticity and viscosity of the synovial fluid. HYVISC is a high molecular weight, injectable HA product developed by Anika Therapeutics (Woburn, Mass.) currently being used to treat osteoarthritis and lameness in racehorses. Other HA-based viscosupplementation products for the treatment of osteoarthritis include SUPARTZ from Seikagaku Corp. (Japan), SUPLASYN from Bioniche Life Sciences, Inc. (Canada), ARTHREASE from DePuy Orthopaedics, Inc. (Warsaw, Ind.), and DUROLANE from Q-Med AB (Sweden).

In one aspect, the compositions of the present invention may be used for the management of osteoarthritis in animals (e.g., horses). It should be noted that some HA products (notably HYVISC by Boehringer Ingelheim Vetmedica, St. Joseph, Mo.) are used in veterinary applications (typically in horses to treat osteoarthritis and lameness).

Other intra-articular compositions used to treat arthritis include corticosteroids. The most common corticosteroids currently used for inflammatory arthritis are methylprednisolone acetate (DEPO-MEDROL, Pharmacia & Upjohn Company, Kalamazoo, Mich.), and triacinolone acetonide (KENALOG, Bristol-Myers Squibb, New York, N.Y.). By adding a fibrosis-inhibiting agent to the intra-articular corticosteroid injection, the intra-articular injection has the added benefit of helping to prevent cartilage breakdown (i.e., it is “chondroprotective”).

Formulations that can be used in these applications include solutions, topical formulations (e.g., solution, cream, ointment, gel) emulsions, micellar solutions, gels (crosslinked and non-crosslinked), suspensions and/or pastes. One form of the formulation is as an injectable composition. For compositions that further contain a polymer to increase the viscosity of the formulation, hyaluronic acid (crosslinked, derivatized and/or non-crosslinked) is an exemplary material. These formulations can further comprise additional polymers (e.g., collagen, poly(ethylene glycol) or dextran) as well as biocompatible solvents (e.g., ethanol, DMSO, or NMP). In one embodiment, the fibrosis-inhibiting therapeutic agent can be incorporated directly into the formulation. In another embodiment, the fibrosis-inhibiting therapeutic agent can be incorporated into a secondary carrier (e.g., micelles, liposomes, emulsions, microspheres, nanospheres etc, as described above). The microsphere and nanospheres may be comprised of degradable polymers. Degradable polymers that can be used include poly(hydroxyl esters) (e.g., PLGA, PLA, PCL, and the like), as well as polyanhydrides, polyorthoesters and polysaccharides (e.g., chitosan and alginates).

In one embodiment, the fibrosis-inhibiting agent further comprises a polymer where the polymer is a degradable polymer. The degradable polymers may include polyesters where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X—Y, Y—X—Y, R—(Y—X)ₙ, R—(X—Y)ₙ and X—Y—X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator. In another embodiment, the fibrosis-inhibiting agent/polymer composition may further comprise a solvent, a liquid oligomer or liquid polymer such that the final composition may be passed through a 18 G needle. The reagents that may be used include ethanol, NMP, PEG 200, PEG 300 and low molecular weight liquid polymers of the form X—Y, Y—X—Y, R—(Y—X)ₙ, R—(X—Y)ₙ and X—Y—X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator.

In another embodiment, the fibrosis-inhibiting agent may be in the form of a solution or suspension in an organic solvent, a liquid oligomer or a liquid polymer. In this embodiment, reagents such as ethanol, NMP, PEG 200, PEG 300 and low molecular weight liquid polymers of the form X—Y, Y—X—Y, R—(Y—X)ₙ, R—(X—Y), and X—Y—X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator, may be used.

Examples of fibrosis-inhibiting agents for use in the treatment of inflammatory arthritis include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for the treatment of inflammatory arthritis will depend on a variety of factors, including the type of formulation and treatment site. However, certain principles can be applied in the application of this art. Drug dose can be calculated as a function of dose per unit area (of the treatment site), total drug dose administered can be measured and appropriate surface concentrations of active drug can be determined. For local application, drugs are to be used at concentrations that range from several times more than to 50%, 20%, 10%, 5%, or even less than 1% of the concentration typically used in a single systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days. In one aspect, the drug is released in effective concentrations for a period ranging from 1-90 days.

The exemplary anti-fibrosing agents, used alone or in combination, should be administered under the following dosing guidelines. The total amount (dose) of anti-scarring agent in the composition can be in the range of about 0.01 μg-10 μg, or 10 μg-10 mg, or 10 mg-250 mg, or 250 mg-1000 mg, or 1000 mg-2500 mg. The dose (amount) of anti-scarring agent per unit area of surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring agents that can be used in conjunction with compositions for the treatment of inflammatory arthritis in accordance with the invention. The following dosages are particularly useful for intra-articular administration: (A) Cell cycle inhibitors including doxorubicin and mitoxantrone. Doxorubicin analogues and derivatives thereof: total dose not to exceed 25 mg (range of 0.1 μg to 25 mg); preferred 1 μg to 5 mg. Dose per unit area of 0.01 μg-100 μg per mm²; preferred dose of 0.1 μg/mm²-10 μg/mm². Minimum concentration of 10⁻⁸-10⁻⁴ M of doxorubicin is to be maintained in the joint. Mitoxantrone and analogues and derivatives thereof: total dose not to exceed 5 mg (range of 0.01 μg to 5 mg); preferred 0.1 μg to 1 mg. Dose per unit area of 0.01 μg-20 μg per mm²; preferred dose of 0.05 μg/mm²-3 μg/mm². Minimum concentration of 10⁻⁸-10⁻⁴ M of mitoxantrone is to be maintained in the joint. (B) Cell cycle inhibitors including paclitaxel and analogues and derivatives (e.g., docetaxel) thereof: total dose not to exceed 10 mg (range of 0.1 μg to 10 mg); preferred 1 μg to 3 mg. Dose per unit area of 0.1 μg-10 μg per mm²; preferred dose of 0.25 μg/mm²-5 μg/mm². Minimum concentration of 10⁻⁸-10⁻⁴ M of paclitaxel is to be maintained in the joint. (C) Cell cycle inhibitors such as podophyllotoxins (e.g., etoposide): total dose not to exceed 10 mg (range of 0.1 μg to 10 mg); preferred 1 μg to 3 mg. Dose per unit area of 0.1 μg-10 μg per mm²; preferred dose of 0.25 μg/mm²-5 μg/mm². Minimum concentration of 10⁻⁸-10⁻⁴ M of etoposide is to be maintained in the joint. (D) Immunomodulators including sirolimus and everolimus. Sirolimus (i.e., rapamycin, RAPAMUNE): total dose not to exceed 10 mg (range of 0.1 μg to 10 mg); preferred 10 μg to 1 mg. Dose per unit area of 0.1 μg-100 μg per mm²; preferred dose of 0.5 μg/mm²-10 μg/mm². Minimum concentration of 10⁻⁸-10⁻⁴ M of sirolimus is to be maintained in the joint. Everolimus and derivatives and analogues thereof: total dose should not exceed 10 mg (range of 0.1 μg to 10 mg); preferred 10 μg to 1 mg. Dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained in the joint. (E) Heat shock protein 90 antagonists (e.g., geldanamycin) and analogues and derivatives thereof: total dose not to exceed 20 mg (range of 0.1 μg to 20 mg); preferred 1 μg to 5 mg. Dose per unit area of 0.1 μg-10 μg per mm 2; preferred dose of 0.25 μg/mm²-5 μg/mm². Minimum concentration of 10⁻⁸-10⁻⁴ M of geldanamycin is to be maintained in the joint. (F) HMGCoA reductase inhibitors (e.g., simvastatin) and analogues and derivatives thereof: total dose not to exceed 2000 mg (range of 10.0 μg to 2000 mg); preferred 10 μg to 300 mg. Dose per unit area of 1.0 μg-1000 μg per mm²; preferred dose of 2.5 μg/mm²-500 μg/mm². Minimum concentration of 10⁻⁸-10⁻³ M of simvastatin is to be maintained in the joint. (G) Inosine monophosphate dehydrogenase inhibitors (e.g., mycophenolic acid, 1-alpha-25 dihydroxy vitamin D₃) and analogues and derivatives thereof: total dose not to exceed 2000 mg (range of 10.0 μg to 2000 mg); preferred 10 [g to 300 mg. Dose per unit area of 1.0 μg-1000 μg per mm²; preferred dose of 2.5 μg/mm²-500 μg/mm². Minimum concentration of 10⁻⁸-10⁻³ M of mycophenolic acid is to be maintained in the joint. (H) NF kappa B inhibitors (e.g., Bay 11-7082) and analogues and derivatives thereof: total dose not to exceed 200 mg (range of 1.0 μg to 200 mg); preferred 1 μg to 50 mg. Dose per unit area of 1.0 μg-100 μg per mm²; preferred dose of 2.5 μg/mm²-50 μg/mm². Minimum concentration of 10⁻⁸-10⁻⁴ M of Bay 11-7082 is to be maintained in the joint. (I) Antimycotic agents (e.g., sulconizole) and analogues and derivatives thereof: total dose not to exceed 2000 mg (range of 10.0 μg to 2000 mg); preferred 10 μg to 300 mg. Dose per unit area of 1.0 μg-1000 μg per mm²; preferred dose of 2.5 μg/mm²-500 μg/mm². Minimum concentration of 10⁻⁸-10⁻³ M of sulconizole is to be maintained in the joint and (J) p38 MAP kinase inhibitors (e.g., SB202190) and analogues and derivatives thereof: total dose not to exceed 2000 mg (range of 10.0 μg to 2000 mg); preferred 10 μg to 300 mg. Dose per unit area of 1.0 μg-1000 μg per mm²; preferred dose of 2.5 μg/mm²-500 μg/mm². Minimum concentration of 10⁻⁸-10⁻³ M of SB202190 is to be maintained in the joint.

In another aspect, systemic treatment may be administered when severe exacerbations or systemic disease (e.g., RA) are present. Anti-scarring agents that are delivered systemically should be dosed according to the level of drug required to inhibit the pathologies of inflammatory arthritis as described above. These systemic doses may vary according to patient, severity of disease, formulation of the administered agent, potency and/or tolerability of the agent, and route of administration. For example, for paclitaxel, doxorubicin or geldanamycin, preferred embodiments would be 10 to 175 mg/m² once every 1 to 4 weeks, 10 to 75 mg/m² daily, as tolerated, or 10 to 175 mg/m² weekly, as tolerated or until symptoms subside. To treat severe acute exacerbations, higher doses of 50 to 250 mg/m² of paclitaxel may be administered as a “pulse” systemic therapy. Other anti-scarring agents can be administered at equivalent doses adjusted for the potency and tolerability of the agent.

According to another aspect, any anti-infective agent described above may be used in conjunction with compositions for the treatment of inflammatory arthritis. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ M to 10⁻⁷ M, or about 10⁻⁷ M to 10⁻⁶ M about 10⁻⁶ M to 10⁻⁵ M or about 10⁻⁴ M of the agent is maintained on the tissue surface.

Prevention of Cartilage Loss (“Chondroprotection”)

In another aspect, polymeric compositions can be used to prevent or reduce the loss of cartilage loss following an injury (e.g., cruciate ligament tear and/or meniscal tear). It has been known for a long time that damage to a joint can predispose a patient to develop osteoarthritis in the joint at a subsequent point in time, but there has been no effective treatment to prevent this occurrence. Instead most of the focus from the medical community and researchers has been on the treatment of the arthritis after it has become established. Treatments for established disease include anti-inflammatory drugs (non-steroidal and steroidal), lubricants or synovial fluid replacements, surgery and joint replacement for severe disease.

Trauma to a joint can take many forms, ranging from a simple sprain which can heal spontaneously to a fracture that creates so many bone fragments that it is almost impossible to reconstruct the joint. The focus for treatment of these injuries revolves around restoring the joint to its normal anatomical state and to resume regular motion. Risk factors for developing arthritis are related to the extent of trauma, the extent of the joint disruption, the degree of the fracture or dislocations, whether or not it is a weight bearing joint, and the characteristic of the joint itself. In general, the greater the trauma to the joint, the greater the risk that the patient will develop osteoarthritis later in life. Surgical correction of a joint to its pre-injury anatomy does not guarantee the prevention of arthritis. In the case of an intra-articular fracture, for example a plateau fracture of the tibia, the treatment is to surgically reconstruct the joint so that it reverts back to a congruent, smooth and intact joint surface with no “step defects” or pieces out of place that would interfere with the gliding of the femur on its surface. Despite improved surgical techniques in repairing these fractures, patients with such fractures have a very high probability of developing degenerative arthritis later on in life.

Anterior cruciate ligament (ACL) injuries in the knee represent a classic example of an injury that predisposes patients to potentially severe degenerative arthritis. The ACL is the ligament that joins the anterior tibial plateau to the posterior femoral intercondylar notch. It is composed of multiple non-parallel fibers with variable fiber lengths that function in bundles to provide tension and mechanical stability to the knee throughout its range of motion. The ACL's stabilizing role has four main functions, including (a) restraining anterior translation of the tibia; (b) preventing hyperextension of the knee; (c) acting as a secondary stabilizer to the valgus stress, reinforcing the medial collateral ligament; and (d) controlling rotation of the tibia on the femur during femoral extensions, and thus, controlling movements such as side-stepping and pivoting. Generally, ACL deficiency results in subluxation of the tibia on the femur causing stretching of the enveloping capsular ligaments and abnormal shear forces on the menisci and on the articular cartilage. Delay in diagnosis and treatment gives rise to increased intra-articular damage as well as stretching of the secondary stabilizing capsular structures.

Despite the known high risk for developing osteoarthritis, patients generally have no associated fractures and have normal x-rays at the time of presentation post-ACL injury. Yet it is well documented that anyone who suffers an ACL injury has a high probability of developing arthritis: 50% by 10 years and 80% by 20 years post-injury. Generally after an ACL rupture patients suffer from instability since the ligament is critical in stabilizing the joint during pivoting and rotation. For example, it is not only required for demanding pivoting sports such as basketball, it is also required for daily activity such as a mother holding her baby as she pivots to get an item from the fridge.

The typical treatment and management of an ACL tear is reconstruction using a graft to replace the torn ACL. The graft may be taken from elsewhere in the patient's extremity (autograft), harvested from a cadaver (allograft) or may be made from a synthetic material. Autograft is the most widely performed orthopedic ACL reconstruction. The technique involves harvesting the patient's own tissue, which may be the mid-third of the patellar tendon with bone attached at both ends, one or two medial hamstrings, or the quadriceps tendon with bone at one end. Synthetic materials have the advantage of being readily available, however, there is a higher failure rate of synthetic grafts compared to autografts and allografts and they have mechanical properties that do not closely resemble the normal ligament. Successful ACL reconstruction is dependent on a number of factors, including surgical technique, post-operative rehabilitation and associated secondary ligament instability. During the surgical procedure, arthroscopy is used to determine whether there are any other associated injuries, which may be treated at the same time, such as meniscal tears or chondral trauma. The surgical procedure is done through a small accessory incision, whereby a tunnel is drilled through the tibia and femur so that the graft may be inserted and fixed.

Surgical reconstruction was initially thought to provide a permanent solution: re-establish a stable knee and prevent degeneration. But other studies demonstrated that after joint injury, there is a cascade of inflammatory activity that once initiated, can be destructive to the joint. This explains why surgical repair itself would have not impact on the prevention of degeneration in traumatized joints; stabilizing a joint or the macro reconstruction of a joint does not address the fundamental underlying biology. Unfortunately, although long-term data has shown that surgery is indeed successful in stabilizing the knee and getting people back to normal activity; it has no impact on the subsequent rate of development of osteoarthritis. As a result, the standard of care to day is to repair the joint acutely and treat the arthritis when it ultimately develops. It should be noted that all joints (in addition to knees) have the potential to become arthritic after trauma, but joints typically involved include; fingers, thumbs, metacarpal (wrist), elbow, shoulder, spine joints (facets, sacroiliac), temperomandibular, otic bones, hips, ankles, tarsal and toes, especially the hallux.

Fibrosis-inhibiting agents such as paclitaxel have demonstrated in animal experiments an ability to prevent cartilage breakdown following cruciate ligament tears. This effect has been seen both in an inflammatory model and biomechanical model of joint injury. In the inflammatory carrageenin-induced arthritis model in rabbits, paclitaxel demonstrated cartilage. Hartley Guinea pigs subjected to surgical transaction of the anterior cruciate ligament represent a mechanical model for arthritis. Typically after the anterior cruciate is severed, the animals develop arthritis within several weeks. The introduction of the fibrosis-inhibiting agent paclitaxel into the joint greatly retarded the arthritic process and protected not only the cartilage, but also the underlying bone, from breakdown.

The present invention addresses a significant unmet medical need: the prevention of progressive joint degeneration after traumatic injury. Introduction of a composition containing a fibrosis-inhibiting agent into a damaged joint shortly after injury, (e.g., through intra-articular injection, peri-articular administration, via arthroscope, as a joint lavage during open surgical procedures) will impact the cascade of events that lead to joint destruction, such as inhibiting inflammation and preventing cartilage matrix destruction. Most ligament injuries are severe enough or painful enough that patients seek immediate medical attention (within the first 24 to 48 hours); long before irreversible changes have occurred in the joint. If at the time of initial presentation to a health care professional, an intra-articular injection of a fibrosis-inhibitor can be administered into the joint to stop or slow down the destructive activity (in the joint and the tissues surrounding the joint), the articular cartilage can be protected from breakdown. Early introduction of the agents of the present invention intervention will slow, decrease or eliminate the cascade of events that lead to osteoarthritis. The invention can be administered immediately after injury, repeated during the period leading up to stabilization surgery, and/or can be administered after surgery is completed.

Thus, within one aspect of the present invention, methods are provided for treating or preventing cartilage loss, comprising the step of administering to a patient in need thereof a therapeutically effective amount of an anti-scarring agent or a composition comprising an anti-scarring agent.

An effective anti-scarring therapy for cartilage loss will accomplish one or more of the following: (i) decrease the severity of symptoms (pain, swelling and tenderness of affected joints; (ii) decrease the severity of clinical signs of the disease (thickening of the joint capsule, synovial hypertrophy, joint effusion, soft tissue contractures, decreased range of motion, ankylosis and fixed joint deformity); (iii) increase the frequency and duration of disease remission/symptom-free periods; (iv) delay or prevent the onset of clinically significant arthritis in a joint that has previously been injured; and/or (v) prevent or reduce fixed impairment and disability.

According to the present invention, any anti-scarring agent described above could be utilized in the practice of this invention. Within certain embodiments of the invention, the composition may release an agent that inhibits one or more of the general components of the process of fibrosis (or scarring) associated with joint damage, including: (a) formation of new blood vessels (angiogenesis), (b) migration and/or proliferation of connective tissue cells (such as fibroblasts or synoviocytes), (c) deposition and remodeling of extracellular matrix (ECM) by matrix metalloproteinase activity, (d) inflammatory response by cytokines (such as IL-1, TNFα, FGF, VEGF). By inhibiting one or more of the components of fibrosis (or scarring), joint damage and osteoarthritis development may be reduced or prevented in a previously injured joint.

In one aspect, the composition includes an anti-scarring agent and a polymeric carrier suitable for application to treat an injured joint. Numerous polymeric and non-polymeric delivery systems and compositions containing an anti-scarring agent for use in the prevention of cartilage loss have been described above. An anti-scarring agent may be administered systemically (orally, intravenously, or by intramuscular or subcutaneous injection) in the minimum dose to achieve the above mentioned results. For patients with only a small number of joints affected, or with disease more prominent in a limited number of joints, the anti-scarring agent can be applied onto tissue within a joint or directly injected into the affected joint (intraarticular injection).

The anti-scarring agent can be administered in any manner described herein. However, preferred methods of administration include intravenous, oral, or subcutaneous, intramuscular or intra-articular injection. The anti-scarring agent can be directly injected into the affected joint (intra-articular injection) via percutaneous needle insertion into the joint capsule, or as part of an arthroscopic procedure performed on the joint. In a preferred embodiment, the intra-articular formulation containing a fibrosis-inhibitor is administered to a joint following an injury with a high probability of inducing subsequent arthritis (e.g., cruciate ligament tears in the knee, meniscal tears in the knee). The fibrosis-inhibiting agent is administered for a period sufficient (either through sustained release preparations and/or repeated injections) to protect the cartilage from breakdown as a result of the injury (or the surgical procedure used to treat it). The anti-scarring agent can be administered as a chronic low dose therapy to prevent disease progression, prolong disease remission, or decrease symptoms in active disease. Alternatively, the therapeutic agent can be administered in higher doses as a “pulse” therapy to induce remission in acutely active disease (such as in the period immediately following a joint injury). The minimum dose capable of achieving these endpoints can be used and can vary according to patient, severity of disease, formulation of the administered agent, clearance from the joint, potency and/or tolerability of the agent, and route of administration.

A variety of commercially available HA compositions for intra-articular injection may be combined with one or more agents according to the present invention including: SYNVISC (Biomatrix, Inc., Ridgefield, N.J.)—an elastoviscous fluid containing hylan (a derivative of sodium hyaluronate (hyaluronan)) polymers derived from rooster combs, HYALGAN (Sanofi-Synthelabo Inc. New York, N.Y.), and ORTHOVISC (Ortho Biotech Products, Bridgewater, N.J.)—a highly purified, high molecular weight, high viscosity injectable form of HA intended to relieve pain and to improve joint mobility and range of motion in patients suffering from osteoarthritis (OA) of the knee. ORTHOVISC is injected into the knee to restore the elasticity and viscosity of the synovial fluid. HYVISC is a high molecular weight, injectable HA product developed by Anika Therapeutics (Woburn, Mass.) currently being used to treat osteoarthritis and lameness in racehorses. Other HA-based viscosupplementation products for intra-articular injection include SUPARTZ from Seikagaku Corp. (Japan), SUPLASYN from Bioniche Life Sciences, Inc. (Canada), ARTHREASE from DePuy Orthopaedics, Inc. (Warsaw, Ind.), and DUROLANE from Q-Med AB (Sweden). By adding a fibrosis-inhibiting agent to the HA, the intra-articular injection has the added benefit of helping to prevent cartilage breakdown (i.e., it is “chondroprotective”).

In one aspect, the compositions of the present invention may be used for the management of osteoarthritis in animals (e.g., horses). It should be noted that some HA products (notably HYVISC by Boehringer Ingelheim Vetmedica, St. Joseph, Mo.) are used in veterinary applications (typically in horses to treat osteoarthritis and lameness).

Fibrosis-inhibiting formulations that can be used for the treatment or prevention of cartilage loss may be in the form of solutions, topical formulations (e.g., solution, cream, ointment, gel) emulsions, micellar solutions, gels (crosslinked and non-crosslinked), suspensions and/or pastes. One form for the formulation is as an injectable composition for intra-articular or arthroscopic delivery. For compositions that further contain a polymer to increase the viscosity of the formulation, hyaluronic acid (crosslinked, derivatized and/or non-crosslinked) is an exemplary material. These formulations can further comprise additional polymers (e.g., collagen, poly(ethylene glycol) or dextran) as well as biocompatible solvents (e.g., ethanol, DMSO, or NMP). In one embodiment, the fibrosis-inhibiting therapeutic agent can be incorporated directly into the formulation. In another embodiment, the fibrosis-inhibiting therapeutic agent can be incorporated into a secondary carrier (e.g., micelles, liposomes, emulsions, microspheres, nanospheres etc, as described above). The microsphere and nanospheres may be comprised of degradable polymers. Degradable polymers that can be used include poly(hydroxyl esters) (e.g., PLGA, PLA, PCL, and the like), as well as polyanhydrides, polyorthoesters and polysaccharides (e.g., chitosan and alginates).

In one embodiment, the fibrosis-inhibiting agent further comprises a polymer where the polymer is a degradable polymer. The degradable polymers may include polyesters where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X—Y, Y—X—Y, R—(Y—X)ₙ, R—(X—Y)ₙ and X—Y—X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator. In another embodiment, the fibrosis-inhibiting agent/polymer composition may further comprise a solvent, a liquid oligomer or liquid polymer such that the final composition may be passed through a 18 G needle. The reagents that may be used include ethanol, NMP, PEG 200, PEG 300 and low molecular weight liquid polymers of the form X—Y, Y—X—Y, R—(Y—X)ₙ, R—(X—Y)ₙ and X—Y—X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator.

In another embodiment, the fibrosis-inhibiting agent may be in the form of a solution or suspension in an organic solvent, a liquid oligomer or a liquid polymer. In this embodiment, reagents such as ethanol, NMP, PEG 200, PEG 300 and low molecular weight liquid polymers of the form X—Y, Y—X—Y, R—(Y—X)ₙ, R—(X—Y)ₙ and X—Y—X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator, may be used.

Examples of fibrosis-inhibiting agents for use in the treatment of, or prevention of, cartilage loss following traumatic injury include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for the treatment of cartilage loss will depend on a variety of factors, including the type of formulation and treatment site. However, certain principles can be applied in the application of this art. Drug dose can be calculated as a function of dose per unit area (of the treatment site), total drug dose administered can be measured and appropriate surface concentrations of active drug can be determined. For local application (such as intra-articular or endoscopic administration), drugs are to be used at concentrations that range from several times more than to 50%, 20%, 10%, 5%, or even less than 1% of the concentration typically used in a single systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days. In one aspect, the drug is released in effective concentrations for a period ranging from 1-90 days.

The exemplary anti-fibrosing agents, used alone or in combination, should be administered under the following dosing guidelines. The total amount (dose) of anti-scarring agent in the composition can be in the range of about 0.01 μg-10 μg, or 10 μg-10 mg, or 10 mg-250 mg, or 250 mg-1000 mg, or 1000 mg-2500 mg. The dose (amount) of anti-scarring agent per unit area of surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring agents that can be used in conjunction with compositions for the treatment of cartilage loss in accordance with the invention. (A) Cell cycle inhibitors including doxorubicin and mitoxantrone. Doxorubicin analogues and derivatives thereof: total dose not to exceed 25 mg (range of 0.1 μg to 25 mg); preferred 1 μg to 5 mg. Dose per unit area of 0.01 μg-100 μg per mm²; preferred dose of 0.1 μg/mm²-10 μg/mm². Minimum concentration of 10⁻⁸-10⁻⁴ M of doxorubicin is to be maintained in the joint. Mitoxantrone and analogues and derivatives thereof: total dose not to exceed 5 mg (range of 0.01 μg to 5 mg); preferred 0.1 μg to 1 mg. Dose per unit area of 0.01 μg-20 μg per mm²; preferred dose of 0.05 μg/mm²-3 μg/mm². Minimum concentration of 10⁻⁸-10⁻⁴ M of mitoxantrone is to be maintained in the joint. (B) Cell cycle inhibitors including paclitaxel and analogues and derivatives (e.g., docetaxel) thereof: total dose not to exceed 10 mg (range of 0.1 μg to 10 mg); preferred 1 μg to 3 mg. Dose per unit area of 0.1 μg-10 μg per mm²; preferred dose of 0.25 μg/mm²-5 μg/mm². Minimum concentration of 10⁻⁸-10⁻⁴ M of paclitaxel is to be maintained in the joint. (C) Cell cycle inhibitors such as podophyllotoxins (e.g., etoposide): total dose not to exceed 10 mg (range of 0.1 μg to 10 mg); preferred 1 μg to 3 mg. Dose per unit area of 0.1 μg-10 μg per mm²; preferred dose of 0.25 μg/mm²-5 μg/mm². Minimum concentration of 10⁻⁸-10⁻⁴ M of etoposide is to be maintained in the joint. (D) Immunomodulators including sirolimus and everolimus. Sirolimus (i.e., rapamycin, RAPAMUNE): total dose not to exceed 10 mg (range of 0.1 μg to 10 mg); preferred 10 μg to 1 mg. Dose per unit area of 0.1 μg-100 μg per mm²; preferred dose of 0.5 μg/mm²-10 μg/mm². Minimum concentration of 10⁻⁸-10⁻⁴ M of sirolimus is to be maintained in the joint. Everolimus and derivatives and analogues thereof: total dose should not exceed 10 mg (range of 0.1 μg to 10 mg); preferred 10 μg to 1 mg. Dose per unit area of 0.1 μg-100 μg per mm² of surface area; preferred dose of 0.3 μg/mm²-10μg/mm². Minimum concentration of 10⁻⁸-10⁻⁴ M of everolimus is to be maintained in the joint. (E) Heat shock protein 90 antagonists (e.g., geldanamycin) and analogues and derivatives thereof: total dose not to exceed 20 mg (range of 0.1 μg to 20 mg); preferred 1 μg to 5 mg. Dose per unit area of 0.1 μg-10 μg per mm²; preferred dose of 0.25 μg/mm²-5 μg/mm². Minimum concentration of 10⁻⁸-10⁻⁴ M of geldanamycin is to be maintained in the joint. (F) HMGCoA reductase inhibitors (e.g., simvastatin) and analogues and derivatives thereof: total dose not to exceed 2000 mg (range of 10.0 μg to 2000 mg); preferred 10 μg to 300 mg. Dose per unit area of 1.0 μg-1000 μg per mm²; preferred dose of 2.5 μg/mm²-500 μg/mm². Minimum concentration of 10⁻⁸-10⁻³ M of simvastatin is to be maintained in the joint. (G) Inosine monophosphate dehydrogenase inhibitors (e.g., mycophenolic acid, 1-alpha-25 dihydroxy vitamin D₃) and analogues and derivatives thereof: total dose not to exceed 2000 mg (range of 10.0 μg to 2000 mg); preferred 10 μg to 300 mg. Dose per unit area of 1.0 μg-1000 μg per mm²; preferred dose of 2.5 μg/mm²-500 μg/mm². Minimum concentration of 10⁻⁸-10⁻³ M of mycophenolic acid is to be maintained in the joint. (H) NF kappa B inhibitors (e.g., Bay 11-7082) and analogues and derivatives thereof: total dose not to exceed 200 mg (range of 1.0 μg to 200 mg); preferred 1 μg to 50 mg. Dose per unit area of 1.0 μg-100 μg per mm²; preferred dose of 2.5 μg/mm²-50 μg/mm². Minimum concentration of 10⁻⁸-10⁻⁴ M of Bay 11-7082 is to be maintained in the joint. (I) Antimycotic agents (e.g., sulconizole) and analogues and derivatives thereof: total dose not to exceed 2000 mg (range of 10.0 μg to 2000 mg); preferred 10 μg to 300 mg. Dose per unit area of 1.0 μg-1000 μg per mm²; preferred dose of 2.5 μg/mm²-500 μg/mm². Minimum concentration of 10⁻⁸-10⁻³ M of sulconizole is to be maintained in the joint and (J) p38 MAP kinase inhibitors (e.g., SB202190) and analogues and derivatives thereof: total dose not to exceed 2000 mg (range of 10.0 μg to 2000 mg); preferred 10 μg to 300 mg. Dose per unit area of 1.0 μg-1000 μg per mm²; preferred dose of 2.5 μg/mm²-500 μg/mm². Minimum concentration of 10⁻⁸-10⁻³ M of SB202190 is to be maintained in the joint.

According to another aspect, any anti-infective agent described above may be used in conjunction with formulations for the treatment or prevention of cartilage loss. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ M to 10⁻⁷ M, or about 10⁻⁷ M to 10⁻⁶ M about 10⁻⁶ M to 10⁻⁵ M or about 10⁻⁵ M to 10⁻⁴ M of the agent is maintained on the tissue surface.

Hypertrophic Scars/Keloids

In another aspect of the invention, compositions containing a therapeutically active agent (e.g., a fibrosis-inhibiting agent) and methods are provided for treating hypertrophic scars and keloids.

Hypertrophic scars and keloids are an overgrowth of dense fibrous tissue that is the result of an excessive fibroproliferative wound healing process. Hypertrophic scars and keloids usually develop after healing of a skin injury. Briefly, healing of wounds and scar formation occurs in three phases: inflammation, proliferation, and maturation. The first phase, inflammation, occurs in response to an injury which is severe enough to break the skin. During this phase, which lasts 3 to 4 days, blood and tissue fluid form an adhesive coagulum and fibrinous network which serves to bind the wound surfaces together. This is then followed by a proliferative phase in which there is ingrowth of capillaries and connective tissue from the wound edges, and closure of the skin defect. Finally, once capillary and fibroblastic proliferation has ceased, the maturation process begins wherein the scar contracts and becomes less cellular, less vascular, and appears flat and white. This final phase may take between 6 and 12 months.

If too much connective tissue is produced and the wound remains persistently cellular, the scar may become red and raised. If the scar remains within the boundaries of the original wound it is referred to as a hypertrophic scar, but if it extends beyond the original scar and into the surrounding tissue, the lesion is referred to as a keloid. Hypertrophic scars and keloids are produced during the second and third phases of scar formation. Several wounds are particularly prone to excessive endothelial and fibroblastic proliferation, including burns, open wounds, and infected wounds. With hypertrophic scars, some degree of maturation occurs and gradual improvement occurs. In the case of keloids however, an actual tumor is produced which can become quite large. Spontaneous improvement in such cases rarely occurs.

Keloids and hypertrophic scars located at most sites are primarily of cosmetic concern; however, some keloids or hypertrophic scars can cause contractures, which may result in a loss of function if overlying a joint, or they can cause significant disfigurement if located on the face. Both keloids and hypertrophic scars can be painful or pruritic.

Within one embodiment of the present invention the polymer compositions are directly injected into a hypertrophic scar or keloid, in order to prevent the progression of these lesions. The frequency of injections will depend upon the release kinetics of the polymer used, and the clinical response. This therapy is of particular value in the prophylactic treatment of conditions which are known to result in the development of hypertrophic scars and keloids (e.g., burns, the excision site of a keloid or hypertrophic scar, wounds on the chest and back of predisposed patients, etc.), and is preferably initiated prior to, or during the proliferative phase (from day 1 forward), but before hypertrophic scar or keloid development (i.e., within the first 3 months post-injury).

In one aspect, the present invention provides topical and injectable compositions that include an anti-scarring agent and a polymeric carrier suitable for application on or into hypertrophic scars or keloids. Numerous polymeric and non-polymeric delivery systems for use in treating hypertrophic scars or keloids have been described above.

Incorporation of a fibrosis-inhibiting agent into a topical formulation or an injectable formulation is one approach to treat this condition. The topical formulation can be in the form of a solution, a suspension, an emulsion, a gel, an ointment, a cream, film or mesh. The injectable formulation can be in the form of a solution, a suspension, an emulsion or a gel. Polymeric and non-polymeric components that can be used to prepare these topical or injectable compositions are described above.

In another embodiment, the fibrosis-inhibiting therapeutic agent can be incorporated into a secondary carrier (e.g., micelles, liposomes, emulsions, microspheres, nanospheres etc, as described above). Microsphere and nanospheres may include degradable polymers. Degradable polymers that can be used include poly(hydroxyl esters) (e.g., PLGA, PLA, PCL, and the like) as well as polyanhydrides, polyorthoesters and polysaccharides (e.g., chitosan and alginates).

In addition, a variety of other compositions and approaches for treating hypertrophic scars and keloids may be used in accordance with the invention. For example, treatment may include the administration of an effective amount of angiogenesis inhibitor (e.g., fumagillol, thalidomide) as a systemic or local treatment to decrease excessive scarring. See, e.g., U.S. Pat. No. 6,638,949. The treatment may be a copolymer composed of a hydrophilic polymer, such as polyethylene glycol, that is bound to a polymer that adsorbs readily to the surfaces of body tissues, such as phenylboronic acid. See, e.g., U.S. Pat. No. 6,596,267. The treatment may include a cryoprobe containing cryogen whereby it is positioned within the hypertrophic scar or keloid to freeze the tissue. See, e.g., U.S. Pat. No. 6,503,246. The treatment may be a method of locally administering an amount of botulinum toxin in or in close proximity to the skin wound, such that the healing is enhanced. See, e.g., U.S. Pat. No. 6,447,787. The treatment may be a liquid composition composed of a film-forming carrier such as a collodion which contains one or more active ingredients such as a topical steroid, silicone gel and vitamin E. See, e.g., U.S. Pat. No. 6,337,076. The treatment may be a method of administering an antifibrotic amount of fluoroquinolone to prevent or treat scar tissue formation. See, e.g., U.S. Pat. No. 6,060,474. The treatment may be a composition of an effective amount of calcium antagonist and protein synthesis inhibitor sufficient to cause matrix degradation at a scar site so as to control scar formation. See, e.g., U.S. Pat. No. 5,902,609. The treatment may be a composition of non-biodegradable microspheres with a substantial surface charge in a pharmaceutically acceptable carrier. See, e.g., U.S. Pat. No. 5,861,149. The treatment may be a composition of endothelial cell growth factor and heparin which may be administered topically or by intralesional injection. See, e.g., U.S. Pat. No. 5,500,409.

Treatments and compositions for hypertrophic scars and keloids, which may be combined with one or more fibrosis-inhibiting agents according to the present invention, include commercially available products. Representative products include, for example, PROXIDERM External Tissue Expansion product for wound healing from Progressive Surgical Products (Westbury, N.Y.), CICA-CARE Gel Sheet dressing product from Smith & Nephew Healthcare Ltd (India), and MEPIFORM Self-Adherent Silicone Dressing from Molnlycke Health Care (Eddystone, Pa.).

In one aspect, the present invention provides topical and injectable compositions that include an anti-scarring agent and a polymeric carrier suitable for application on or into hypertrophic scars or keloids or sites that are prone to forming hypertrophic scars or keloids.

Within one embodiment of the present invention either anti-scarring agents alone, or anti-scarring compositions as described above, are directly injected into a hypertrophic scar or keloid, in order to prevent the progression of these lesions. The frequency of injections will depend upon the release kinetics of the polymer used (if present), and the clinical response. This therapy is of particular value in the prophylactic treatment of conditions which are known to result in the development of hypertrophic scars and keloids (e.g., burns, the excision site of a keloid or hypertrophic scar, wounds on the chest and back of predisposed patients, etc.), and is preferably initiated prior to, or during the proliferative phase (from day 1 forward), but before hypertrophic scar or keloid development (i.e., within the first 3 months post-injury).

According to the present invention, any fibrosis-inhibiting agent described above could be utilized alone or in combination in the practice of this embodiment. Within one embodiment of the invention, compositions for treating hypertrophic scars or keloids may release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue).

Examples of fibrosis-inhibiting agents for use in composition for treating hypertrophic scars and keloids include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for the treatment of hypertrophic scars and keloids will depend on a variety of factors, including the type of formulation and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days. In one aspect, the drug is released in effective concentrations for a period ranging from 1-90 days.

The exemplary anti-fibrosing agents, used alone or in combination, should be administered under the following dosing guidelines. The total amount (dose) of anti-scarring agent in the composition can be in the range of about 0.01 μg-10 μg, or 10 μg-10 mg, or 10 mg-250 mg, or 250 mg-1000 mg, or 1000 mg-2500 mg. The dose (amount) of anti-scarring agent per unit area of surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring agents that can be used in conjunction with compositions for treating hypertrophic scars and keloids in accordance with the invention. (A) Cell cycle inhibitors including doxorubicin and mitoxantrone. Doxorubicin analogues and derivatives thereof: total dose not to exceed 25 mg (range of 0.1 μg to 25 mg); preferred 1 μg to 5 mg. Dose per unit area of 0.01 μg-100 μg per mm²; preferred dose of 0.1 μg/mm²-10 μg/mm². Minimum concentration of 10⁻⁸-10⁻⁴ M of doxorubicin is to be maintained in the wound, keloid or hypertrophic scar. Mitoxantrone and analogues and derivatives thereof: total dose not to exceed 5 mg (range of 0.01 μg to 5 mg); preferred 0.1 μg to 1 mg. Dose per unit area of 0.01 μg-20 μg per mm 2; preferred dose of 0.05 μg/mm²-3 μg/mm². Minimum concentration of 10⁻⁸-10⁻⁴ M of mitoxantrone is to be maintained in the wound, keloid or hypertrophic scar. (B) Cell cycle inhibitors including paclitaxel and analogues and derivatives (e.g., docetaxel) thereof: total dose not to exceed 10 mg (range of 0.1 μg to 10 mg); preferred 1 μg to 3 mg. Dose per unit area of 0.1 μg-10 μg per mm²; preferred dose of 0.25 μg/mm²-5 μg/mm². Minimum concentration of 10⁻⁸-10⁻⁴ M of paclitaxel is to be maintained in the wound, keloid or hypertrophic scar. (C) Cell cycle inhibitors such as podophyllotoxins (e.g., etoposide): total dose not to exceed 10 mg (range of 0.1 μg to 10 mg); preferred 1 μg to 3 mg. Dose per unit area of 0.1 μg-10 μg per mm²; preferred dose of 0.25 μg/mm²-5 μg/mm². Minimum concentration of 10⁻⁸-10⁻⁴ M of etoposide is to be maintained in the wound, keloid or hypertrophic scar. (D) Immunomodulators including sirolimus and everolimus. Sirolimus (i.e., rapamycin, RAPAMUNE): total dose not to exceed 10 mg (range of 0.1 μg to 10 mg); preferred 10 μg to 1 mg. Dose per unit area of 0.1 μg-100 μg per mm²; preferred dose of 0.5 μg/mm²-10 μg/mm². Minimum concentration of 10⁻⁸-10⁻⁴ M of sirolimus is to be maintained in the wound, keloid or hypertrophic scar. Everolimus and derivatives and analogues thereof: total dose should not exceed 10 mg (range of 0.1 μg to 10 mg); preferred 10 μg to 1 mg. Dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained in the wound, keloid or hypertrophic scar. (E) Heat shock protein 90 antagonists (e.g., geldanamycin) and analogues and derivatives thereof: total dose not to exceed 20 mg (range of 0.1 μg to 20 mg); preferred 1 μg to 5 mg. Dose per unit area of 0.1 μg-10 μg per mm²; preferred dose of 0.25 μg/mm²-5 μg/mm². Minimum concentration of 10⁻⁸-10⁻⁴ M of geldanamycin is to be maintained in the wound, keloid or hypertrophic scar. (F) HMGCoA reductase inhibitors (e.g., simvastatin) and analogues and derivatives thereof: total dose not to exceed 2000 mg (range of 10.0 μg to 2000 mg); preferred 10 μg to 300 mg. Dose per unit area of 1.0 μg-1000 μg per mm²; preferred dose of 2.5 μg/mm²-500 μg/mm². Minimum concentration of 10⁻⁸-10⁻³ M of simvastatin is to be maintained in the wound, keloid or hypertrophic scar. (G) Inosine monophosphate dehydrogenase inhibitors (e.g., mycophenolic acid, 1-alpha-25 dihydroxy vitamin D₃) and analogues and derivatives thereof: total dose not to exceed 2000 mg (range of 10.0 μg to 2000 mg); preferred 10 μg to 300 mg. Dose per unit area of 1.0 μg-1000 μg per mm²; preferred dose of 2.5 μg/mm²-500 μg/mm². Minimum concentration of 10⁻⁸-10⁻³ M of mycophenolic acid is to be maintained in the wound, keloid or hypertrophic scar. (H) NF kappa B inhibitors (e.g., Bay 11-7082) and analogues and derivatives thereof: total dose not to exceed 200 mg (range of 1.0 μg to 200 mg); preferred 1 μg to 50 mg. Dose per unit area of 1.0 μg-100 μg per mm²; preferred dose of 2.5 μg/mm²-50 μg/mm². Minimum concentration of 10⁻⁸-10⁻⁴ M of Bay 11-7082 is to be maintained in the wound, keloid or hypertrophic scar. (I) Antimycotic agents (e.g., sulconizole) and analogues and derivatives thereof: total dose not to exceed 2000 mg (range of 10.0 μg to 2000 mg); preferred 10 μg to 300 mg. Dose per unit area of 1.0 μg-1000 μg per mm²; preferred dose of 2.5 μg/mm²-500 μg/mm². Minimum concentration of 10⁻⁸-10⁻³ M of sulconizole is to be maintained in the wound, keloid or hypertrophic scar and (J) p38 MAP kinase inhibitors (e.g., SB202190) and analogues and derivatives thereof: total dose not to exceed 2000 mg (range of 10.0 μg to 2000 mg); preferred 10 μg to 300 mg. Dose per unit area of 1.0 μg-1000 μg per mm²; preferred dose of 2.5 μg/mm²-500 μg/mm². Minimum concentration of 10⁻⁸-10⁻³ M of SB202190 is to be maintained in the wound, keloid or hypertrophic scar.

According to another aspect, any anti-infective agent described above may be used in conjunction with formulations for the treatment or prevention of hypertrophic scars and keloids. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ M to 10⁻⁷ M, or about 10⁻⁷ M to 10⁻⁶ M about 10⁻⁶ M to 10⁻⁵M or about 10⁻⁵ M to 10⁻⁴ M to the agent is maintained on the tissue surface.

Vascular Disease

In one aspect, the present invention provides for the use of a polymer composition comprising a polymeric carrier and one or more fibrosis-inhibiting agents for the treatment of vascular disease (e.g., stenosis, restenosis, or atherosclerosis).

Perivascular Delivery

A further aspect of the invention provides therapeutic compositions which may be delivered perivascularly (e.g., to an external portion of a blood vessel or directly into the adventitia of a blood vessel) for the treatment or prevention of a vascular disease (e.g., stenosis, restenosis, or atherosclerosis).

Perivascular drug delivery involves percutaneous administration of localized (often sustained release) therapeutic formulations using a needle or catheter directed via ultrasound, CT, fluoroscopic, MRI or endoscopic guidance to the adventitial surface of a targeted blood vessel (arteries, veins, autologous bypass grafts, synthetic bypass grafts, AV fistulas). Alternatively the procedure can be performed intra-operatively (e.g., during bypass surgery, hemodialysis access surgery) under direct vision or with additional imaging guidance. Such a procedure can also be performed in conjunction with endovascular procedures such as angioplasty, atherectomy, or stenting or in association with an operative arterial procedure such as endarterectomy, vessel or graft repair or graft insertion.

For example, within one embodiment, polymeric paclitaxel formulations can be injected into the vascular wall or applied to the adventitial surface of a blood vessel allowing drug concentrations to remain highest in regions where biological activity is most needed. This has the potential to reduce local “washout” of the drug that can be accentuated by continuous blood flow over the surface of an endovascular drug delivery device (such as a drug-coated stent). Administration of effective fibrosis-inhibiting agents to the external surface of the vessel can reduce obstruction of the artery, vein or graft and reduce the risk of complications associated with intravascular manipulations (such as restenosis, embolization, thrombosis, plaque rupture, and systemic drug toxicity).

For example, in a patient with narrowing of the superficial femoral artery, balloon angioplasty would be performed in the usual manner (i.e., passing a balloon angioplasty catheter down the artery over a guide wire and inflating the balloon across the lesion). Prior to, at the time of, or after angioplasty, a needle would be inserted through the skin under ultrasound, fluoroscopic, or CT guidance and a fibrosis-inhibiting agent or composition (e.g., paclitaxel impregnated into a slow release polymer) would be infiltrated through the needle or catheter in a circumferential manner directly around the area of narrowing in the artery. This could be performed around any artery, vein or graft, but ideal candidates for this intervention include diseases of the carotid, coronary, iliac, common femoral, superficial femoral and popliteal arteries and at the site of graft anastomosis. Logical venous sites include infiltration around veins in which indwelling catheters are inserted. Similarly at the time of endoscopic or open coronary bypass surgery, peripheral bypass surgery or hemodialysis access surgery, a fibrosis-inhibiting agent or composition (e.g., paclitaxel impregnated into a slow release polymer) would be infiltrated, sprayed or wrapped in a circumferential manner in the region of the anastomosis where there is an increased incidence of restenosis. This could be performed around any artery, vein or graft, but ideal candidates for this intervention include diseases of the carotid, coronary, iliac, common femoral, superficial femoral and popliteal arteries and at the site of AV graft anastomosis.

According to the present invention, any anti-scarring agent described above can be utilized in the practice of this invention. Within one embodiment, compositions for perivascular drug delivery may be adapted to release an agent that inhibits one or more of the five general components of the process of fibrosis (or scarring), including: inflammatory response and inflammation, migration and proliferation of connective tissue cells (such as fibroblasts or smooth muscle cells), formation of new blood vessels (angiogenesis), deposition of extracellular matrix (ECM), and remodeling (maturation and organization of the fibrous tissue). By inhibiting one or more of the components of fibrosis (or scarring), the overgrowth of neointimal tissue may be inhibited or reduced.

The drug dose of the fibrosis-inhibiting agent administered from the present compositions for perivascular delivery will depend on a variety of factors, including the type of formulation and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days. In one aspect, the drug is released in effective concentrations for a period ranging from 1-90 days.

Several examples of fibrosis-inhibiting agents for use with compositions for perivascular drug delivery include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The exemplary anti-fibrosing agents, used alone or in combination, should be administered under the following dosing guidelines. The total amount (dose) of anti-scarring agent in the composition can be in the range of about 0.01 μg-10 μg, or 10 μg-10 mg, or 10 mg-250 mg, or 250 mg-1000 mg, or 1000 mg-2500 mg. The dose (amount) of anti-scarring agent per unit area of surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring agents that can be used in conjunction with perivascular administration in accordance with the invention. (A) Cell cycle inhibitors including doxorubicin and mitoxantrone. Doxorubicin analogues and derivatives thereof: total dose not to exceed 25 mg (range of 0.1 μg to 25 mg); preferred 1 μg to 5 mg. The dose per unit area of the implant is 0.01 μg-100 μg per mm²; preferred dose of 0.1 μg/mm²-10 μg/mm². Minimum concentration of 10⁻⁸-10⁻⁴ M of doxorubicin is to be maintained on the adventitial surface of the artery, vein or graft. Mitoxantrone and analogues and derivatives thereof: total dose not to exceed 5 mg (range of 0.01 μg to 5 mg); preferred 0.1 μg to 1 mg. The dose per unit area of the implant is 0.01 μg-20 μg per mm²; preferred dose of 0.05 μg/mm²-3 μg/mm². Minimum concentration of 10⁻⁸-10⁻⁴ M of mitoxantrone is to be maintained on the adventitial surface of the artery, vein or graft. (B) Cell cycle inhibitors including paclitaxel and analogues and derivatives (e.g., docetaxel) thereof: total dose not to exceed 10 mg (range of 0.1 μg to 10 mg); preferred 1 μg to 3 mg. The dose per unit area of the implant is 0.1 μg-10 μg per mm²; preferred dose of 0.25 μg/mm²-5 μg/mm². Minimum concentration of 10⁻⁸-10⁻⁴ M of paclitaxel is to be maintained on the adventitial surface of the artery, vein or graft. (C) Cell cycle inhibitors such as podophyllotoxins (e.g., etoposide): total dose not to exceed 10 mg (range of 0.1 μg to 10 mg); preferred 1 μg to 3 mg. The dose per unit area of the implant is 0.1 μg-10 μg per mm²; preferred dose of 0.25 μg/mm²-5 μg/mm². Minimum concentration of 10⁻⁸-10⁻⁴ M of etoposide is to be maintained on the adventitial surface of the artery, vein or graft. (D) Immunomodulators including sirolimus and everolimus. Sirolimus (.i.e., rapamycin, RAPAMUNE): Total dose not to exceed 10 mg (range of 0.1 μg to 10 mg); preferred 10 μg to 1 mg. The dose per unit area of the implant is 0.1 μg-100 μg per mm²; preferred dose of 0.5 μg/mm²-10 μg/mm². Minimum concentration of 10⁻⁸-10⁻⁴ M of sirolimus is to be maintained on the adventitial surface of the artery, vein or graft. Everolimus and derivatives and analogues thereof: total dose should not exceed 10 mg (range of 0.1 μg to 10 mg); preferred 10 μg to 1 mg. The dose per unit area of the implant is 0.1 μg-100 μg per mm² of surface area; preferred dose of 0.3 μg/mm²-10 μg/mm². Minimum concentration of 10⁻⁸-10⁻⁴ M of everolimus is to be maintained on the adventitial surface of the artery, vein or graft. (E) Heat shock protein 90 antagonists (e.g., geldanamycin) and analogues and derivatives thereof: total dose not to exceed 20 mg (range of 0.1 μg to 20 mg); preferred 1 μg to 5 mg. The dose per unit area of the implant is 0.1 μg-10 μg per mm²; preferred dose of 0.25 μg/mm²-5 μg/mm². Minimum concentration of 10⁻⁸-10⁻⁴ M of geldanamycin is to be maintained on the adventitial surface of the artery, vein or graft. (F) HMGCoA reductase inhibitors (e.g., simvastatin) and analogues and derivatives thereof: total dose not to exceed 2000 mg (range of 10.0 μg to 2000 mg); preferred 10 μg to 300 mg. The dose per unit area of the implant is 1.0 μg-1000 μg per mm²; preferred dose of 2.5 μg/mm²-500 μg/mm². Minimum concentration of 10⁻⁸-10⁻³ M of simvastatin is to be maintained on the adventitial surface of the artery, vein or graft. (G) Inosine monophosphate dehydrogenase inhibitors (e.g., mycophenolic acid, 1-alpha-25 dihydroxy vitamin D₃) and analogues and derivatives thereof: total dose not to exceed 2000 mg (range of 10.0 μg to 2000 mg); preferred 10 μg to 300 mg. The dose per unit area of the implant is 1.0 μg-1000 μg per mm²; preferred dose of 2.5 μg/mm²-500 μg/mm². Minimum concentration of 10⁻⁸-10⁻³ M of mycophenolic acid is to be maintained on the adventitial surface of the artery, vein or graft. (H) NF kappa B inhibitors (e.g., Bay 11-7082) and analogues and derivatives thereof: total dose not to exceed 200 mg (range of 1.0 μg to 200 mg); preferred 1 μg to 50 mg. The dose per unit area of the implant is 1.0 μg-100 μg per mm²; preferred dose of 2.5 μg/mm²-50 μg/mm². Minimum concentration of 10⁻⁸-10⁻⁴ M of Bay 11-7082 is to be maintained on the adventitial surface of the artery, vein or graft. (I) Antimycotic agents (e.g., sulconizole) and analogues and derivatives thereof: total dose not to exceed 2000 mg (range of 10.0 μg to 2000 mg); preferred 10 μg to 300 mg. The dose per unit area of the implant is 1.0 μg-1000 μg per mm²; preferred dose of 2.5 μg/mm²-500 μg/mm². Minimum concentration of 10⁻⁸-10⁻³ M of sulconizole is to be maintained on the adventitial surface of the artery, vein or graft and (J) p38 MAP kinase inhibitors (e.g., SB202190) and analogues and derivatives thereof: total dose not to exceed 2000 mg (range of 10.0 μg to 2000 mg); preferred 10 μg to 300 mg. The dose per unit area of the implant is 1.0 μg-1000 μg per mm²; preferred dose of 2.5 μg/mm²-500 μg/mm². Minimum concentration of 10⁻⁸-10⁻³ M of SB202190 is to be maintained on the adventitial surface of the artery, vein or graft.

According to another aspect, any anti-infective agent described above may be used alone or in conjunction with a fibrosing agent in the practice of the present embodiment. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ M to 10⁻⁷ M, or about 10⁻⁷ M to 10⁻⁶ M about 10⁻⁶ M to 10⁻⁵ M or about 10⁻⁵ M to 10⁻⁴ M of the agent is maintained on the tissue surface.

Coating Material for Medical Devices and Implants

The fibrosis-inhibiting agents and compositions of the present invention can also be combined with an implant or an implantable medical device, (e.g., artificial joints, retaining pins, cranial plates, and the like, of metal, plastic and/or other materials), breast implants (e.g., silicone gel envelopes, foam forms, and the like), implanted catheters and cannulas intended for long-term use (beyond about three days), artificial organs and vessels (e.g., artificial hearts, pancreases, kidneys, blood vessels, and the like), drug delivery devices (including monolithic implants, pumps and controlled release devices such as ALZET minipumps (DURECT Corporation, Cupertino, Calif.), steroid pellets for anabolic growth or contraception, and the like, sutures for dermal or internal use, periodontal membranes, ophthalmic shields, corneal lenticules, and the like.

Another use of the fibrosis-inhibiting compounds and compositions is as a coating material for synthetic implants. In a general method for coating a surface of a synthetic implant, the multifunctional compounds are exposed to the modified environment, and a thin layer of the composition is then applied to a surface of the implant before substantial inter-reaction has occurred. In one embodiment, in order to minimize cellular and fibrous reaction to the coated implant, the compounds are selected so as to result in a matrix that has a net neutral charge. Application of the compounds to the implant surface may be by extrusion, brushing, spraying, or by any other convenient means. Following application of the compounds to the implant surface, inter-reaction is allowed to continue until complete and the three-dimensional matrix is formed.

Although this method can be used to coat the surface of any type of synthetic implant, it is particularly useful for implants where reduced thrombogenicity is an important consideration, such as artificial blood vessels and heart valves, vascular grafts, vascular stents, anastomotic connector devices, and stent/graft combinations. The method may also be used to coat implantable surgical membranes (e.g., monofilament polypropylene) or meshes (e.g., for use in hernia repair). Breast implants may also be coated using the above method in order to minimize capsular contracture.

The fibrosis-inhibiting compounds and compositions can also be coated on a suitable fibrous material, which can then be wrapped around a bone to provide structural integrity to the bone. The term “suitable fibrous material” as used herein, refers to a fibrous material which is substantially insoluble in water, non-immunogenic, biocompatible, and immiscible with the crosslinkable compositions of the invention. The fibrous material may comprise any of a variety of materials having these characteristics and may be combined with crosslinkable compositions herein in order to form and/or provide structural integrity to various implants or devices used in connection with medical and pharmaceutical uses.

The fibrosis-inhibiting compounds and compositions of the present invention may also be used to coat lenticules, which are made from either naturally occurring or synthetic polymers.

Representative examples of medical devices which may be coated using the polymer compositions of the invention include vascular stents, gastrointestinal stents, tracheal/bronchial stents, genital-urinary stents, ENT stents, intra-articular implants, intraocular lenses, implants for hypertrophic scars and keloids, vascular grafts, anastomotic connector devices, implantable sensors, implantable pumps, implantable electrical devices, such as implantable neurostimulators, implantable electrical leads, surgical adhesion barriers, glaucoma drainage devices, film or mesh, prosthetic heart valves, tympanostomy tubes, penile implants, endotracheal and tracheostomy tubes, peritoneal dialysis catheters, intracranial pressure monitors, vena cava filters, CVCs, ventricular assist device (e.g., LVAD), spinal prostheses, urinary (Foley) catheters, prosthetic bladder sphincters, orthopedic implants, and gastrointestinal drainage tubes.

Infiltration of Polymeric Compositions Around Medical Devices and Implants

Another use of the polymer compositions described herein may be to infiltrate the composition into tissue adjacent to a medical device. The subject polymer compositions may contain an anti-fibrotic and/or anti-infective agent.

Polymeric compositions may be infiltrated around implanted medical devices by applying the composition directly and/or indirectly into and/or onto (a) tissue adjacent to the medical device; (b) the vicinity of the medical device-tissue interface; (c) the region around the medical device; and (d) tissue surrounding the medical device. Methods for infiltrating the subject polymer compositions into tissue adjacent to a medical device include delivering the polymer composition: (a) to the medical device surface (e.g., as an injectable, paste, gel or mesh) during the implantation procedure; (b) to the surface of the tissue (e.g., as an injectable, paste, gel, in situ forming gel or mesh) immediately prior to, or during, implantation of the medical device; (c) to the surface of the medical device and/or the tissue surrounding the implanted medical device (e.g., as an injectable, paste, gel, in situ forming gel or mesh) immediately after the implantation of the medical device; (d) by topical application of the composition into the anatomical space where the medical device may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the medical device as a solution as an infusate or as a sustained release preparation; (f) by any combination of the aforementioned methods. Combination therapies (e.g., combinations of therapeutic agents and combinations with antithrombotic and/or antiplatelet agents) may also be used. In all cases it is understood that the subject polymer compositions may be infiltrated into tissue adjacent to all or a portion of the device.

Representative examples of polymer compositions that may be infiltrated into tissue adjacent to a medical device include: (a) sprayable collagen-containing formulations such as COSTASIS (Angiotech Pharmaceuticals, Inc., Canada) and crosslinked poly(ethylene glycol)—methylated collagen compositions (described, e.g., in U.S. Pat. Nos. 5,874,500 and 5,565,519), either alone, or loaded with a therapeutic agent (e.g., an anti-scarring and/or anti-infective agent), infiltrated into tissue adjacent to the medical device; (b) sprayable PEG-containing formulations such as COSEAL (Angiotech Pharmaceuticals, Inc.), FOCALSEAL (Genzyme Corporation, Cambridge, Mass.), SPRAYGEL or DURASEAL (both from Confluent Surgical, Inc., Boston, Mass.), either alone, or loaded with a therapeutic agent (e.g., an anti-scarring and/or anti-infective agent), infiltrated into tissue adjacent to the medical device; (c) fibrinogen-containing formulations such as FLOSEAL or TISSEAL (both from Baxter Healthcare Corporation, Fremont, Calif.), either alone, or loaded with a therapeutic agent (e.g., an anti-scarring and/or anti-infective agent), infiltrated into tissue adjacent to the medical device; (d) hyaluronic acid-containing formulations such as RESTYLANE or PERLANE (both from Q-Med AB, Sweden), HYLAFORM (Inamed Corporation, Santa Barbara, Calif.), SYNVISC (Biomatrix, Inc., Ridgefield, N.J.), SEPRAFILM or SEPRACOAT (both from Genzyme Corporation), either alone, or loaded with a therapeutic agent (e.g., an anti-scarring and/or anti-infective agent), infiltrated into tissue adjacent to the medical device; (e) polymeric gels for surgical implantation such as REPEL (Life Medical Sciences, Inc., Princeton, N.J.) or FLOWGEL (Baxter Healthcare Corporation), either alone, or loaded with a therapeutic agent (e.g., an anti-scarring and/or anti-infective agent), infiltrated into tissue adjacent to the medical device; (f) orthopedic “cements” used to hold prostheses and tissues in place, either alone, or loaded with a therapeutic agent (e.g., an anti-scarring and/or anti-infective agent), infiltrated into tissue adjacent to the medical device, such as OSTEOBOND (Zimmer, Inc., Warsaw, Ind.), low viscosity cement (LVC); Wright Medical Technology, Inc., Arlington, Tenn.), SIMPLEX P (Stryker Corporation, Kalamazoo, Mich.), PALACOS (Smith & Nephew Corporation, United Kingdom), and ENDURANCE (Johnson & Johnson, Inc., New Brunswick, N.J.); (g) surgical adhesives containing cyanoacrylates such as DERMABOND (Johnson & Johnson, Inc.), INDERMIL (U.S. Surgical Company, Norwalk, Conn.), GLUSTITCH (Blacklock Medical Products Inc., Canada), TISSUEMEND (Veterinary Products Laboratories, Phoenix, Ariz.), VETBOND (3M Company, St. Paul, Minn.), HISTOACRYL BLUE (Davis & Geck, St. Louis, Mo.) and ORABASE SOOTHE-N-SEAL LIQUID PROTECTANT (Colgate-Palmolive Company, New York, N.Y.), either alone, or loaded with a therapeutic agent (e.g., an anti-scarring and/or anti-infective agent), infiltrated into tissue adjacent to the medical device; (h) implants containing hydroxyapatite (or synthetic bone material such as calcium sulfate, VITOSS and CORTOSS (both from Orthovita, Inc., Malvern, Pa.), either alone, or loaded with a therapeutic agent (e.g., an anti-scarring and/or anti-infective agent), infiltrated into tissue adjacent to the medical device; (i) other biocompatible tissue fillers, such as those made by BioCure, Inc. (Norcross, Ga.), 3M Company (St. Paul, Minn.) and Neomend, Inc. (Sunnyvale, Calif.), either alone, or loaded with a therapeutic agent (e.g., an anti-scarring and/or anti-infective agent), infiltrated into tissue adjacent to the medical device; (j) polysacharride gels such as the ADCON series-of gels (available from Gliatech, Inc., Cleveland, Ohio) either alone, or loaded with a therapeutic agent (e.g., an anti-scarring and/or anti-infective agent), infiltrated into tissue adjacent to the medical device; and/or (k) films, sponges or meshes such as INTERCEED (Gynecare Worldwide, a division of Ethicon, Inc., Somerville, N.J.), VICRYL mesh (Ethicon, Inc.), and GELFOAM (Pfizer, Inc., New York, N.Y.), either alone, or loaded with a therapeutic agent (e.g., an anti-scarring and/or anti-infective agent), infiltrated into tissue adjacent to the medical device.

Other examples of polymer compositions that may be infiltrated into tissue adjacent to a medical device include compositions formed from reactants comprising either one or both of pentaerythritol poly(ethylene glycol)ether tetra-sulfhydryl] (4-armed thiol PEG, which includes structures having a linking group(s) between a sulfhydryl group(s) and the terminus of the polyethylene glycol backbone) and pentaerythritol poly(ethylene glycol)ether tetra-succinimidyl glutarate] (4-armed NHS PEG, which again includes structures having a linking group(s) between a NHS group(s) and the terminus of the polyethylene glycol backbone) as reactive reagents. Another preferred composition comprises either one or both of pentaerythritol poly(ethylene glycol)ether tetra-amino] (4-armed amino PEG, which includes structures having a linking group(s) between an amino group(s) and the terminus of the polyethylene glycol backbone) and pentaerythritol poly(ethylene glycol)ether tetra-succinimidyl glutarate] (4-armed NHS PEG, which again includes structures having a linking group(s) between a NHS group(s) and the terminus of the polyethylene glycol backbone) as reactive reagents. Chemical structures for these reactants are shown in, e.g., U.S. Pat. 5,874,500. Optionally, collagen or a collagen derivative (e.g., methylated collagen) is added to the poly(ethylene glycol)-containing reactant(s) to form a preferred crosslinked matrix.

Representative examples of medical devices for use with the subject compositions are described below.

Intravascular Devices

In one aspect, the subject polymer compositions may be infiltrated into tissue adjacent to an intravascular device. The subject polymer compositions may contain a therapeutic agent (e.g., an anti-scarring and/or anti-infective agent). “Intravascular devices” refers to devices that are implanted at least partially within the vasculature (e.g., blood vessels). Examples of intravascular devices that may be used in the present invention include, e.g., catheters, balloon catheters, balloons, stents, covered stents, stent grafts, anastomotic connectors, and guidewires.

In another aspect, the subject polymer compositions may be infiltrated into tissue adjacent to an intravascular stent. “Stent” refers to devices comprising a cylindrical tube (composed of a metal, textile, non-degradable or degradable polymer, and/or other suitable material (such as biological tissue) which maintains the flow of blood from one portion of a blood vessel to another. In one aspect, a stent is an endovascular scaffolding which maintains the lumen of a body passageway (e.g., an artery) and allows bloodflow. Representative examples of stents that may benefit from having the subject polymer composition infiltrated into adjacent tissue include vascular stents, such as coronary stents, peripheral stents, and covered stents.

Stents that may be used in the present invention include metallic stents, polymeric stents, biodegradable stents and covered stents. Stents may be self-expandable or balloon-expandable, composed of a variety of metal compounds and/or polymeric materials, fabricated in innumerable designs, used in coronary or peripheral vessels, composed of degradable and/or nondegradable components, fully or partially covered with vascular graft materials (so called “covered stents”) or “sleeves”, and may be bare metal or drug-eluting.

Stents may be comprise a metal or metal alloy such as stainless steel, spring tempered stainless steel, stainless steel alloys, gold, platinum, super elastic alloys, cobalt-chromium alloys and other cobalt-containing alloys (including ELGILOY (Combined Metals of Chicago, Grove Village, Ill.), PHYNOX (Alloy Wire International, United Kingdom) and CONICHROME (Carpenter Technology Corporation, Wyomissing, Pa.)), titanium-containing alloys, platinum-tungsten alloys, nickel-containing alloys, nickel-titanium alloys (including nitinol), malleable metals (including tantalum); a composite material or a clad composite material and/or other functionally equivalent materials; and/or a polymeric (non-biodegradable or biodegradable) material. Representative examples of polymers that may be included in the stent construction include polyethylene, polypropylene, polyurethanes, polyesters, such as polyethylene terephthalate (e.g., DACRON or MYLAR (E. I. DuPont De Nemours and Company, Wilmington, Del.)), polyamides, polyaramids (e.g., KEVLAR from E.I. DuPont De Nemours and Company), polyfluorocarbons such as poly(tetrafluoroethylene with and without copolymerized hexafluoropropylene) (available, e.g., under the trade name TEFLON (E. I. DuPont De Nemours and Company), silk, as well as the mixtures, blends and copolymers of these polymers. Stents also may be made with engineering plastics, such as thermotropic liquid crystal polymers (LCP), such as those formed from p,p′-dihydroxy-polynuclear-aromatics or dicarboxy-polynuclear-aromatics.

Further types of stents that may be used in the present invention are described, e.g., in PCT Publication No. WO 01/01957 and U.S. Pat. Nos. 6,165,210; 6,099,561; 6,071,305; 6,063,101; 5,997,468; 5,980,551; 5,980,566; 5,972,027; 5,968,092; 5,951,586; 5,893,840; 5,891,108; 5,851,231; 5,843,172; 5,837,008; 5,766,237; 5,769,883; 5,735,811; 5,700,286; 5,683,448; 5,679,400; 5,665,115; 5,649,977; 5,637,113; 5,591,227; 5,551,954; 5,545,208; 5,500,013; 5,464,450; 5,419,760; 5,411,550; 5,342,348; 5,286,254; and 5,163,952. Removable drug-eluting stents are described, e.g., in Lambert, T. (1993) J. Am. Coll. Cardiol.: 21: 483A. Moreover, the stent may be adapted to release a therapeutic agent, for example, at only the distal ends, or along the entire body of the stent.

Balloon over stent devices, such as are described in Wilensky, R. L. (1993) J. Am. Coll. Cardiol.: 21: 185A, also are suitable for having the subject polymer composition infiltrated into adjacent tissue.

In addition to using the more traditional stents, stents that are specifically designed for drug delivery may be used. Examples of these specialized drug delivery stents as well as traditional stents include those from Conor Medsystems (Palo Alto, Calif.) (e.g., U.S. Pat. Nos. 6,527,799; 6,293,967; 6,290,673; 6,241,762; U.S. patent application Publication Nos. 2003/0199970 and 2003/0167085; and PCT Publication No. WO 03/015664).

Examples of intravascular stents, which may have the subject polymer composition infiltrated into adjacent tissue according to the present invention, include commercially available products. The stent may be self-expanding or balloon expandable (e.g., STRECKER stent by Medi-Tech/Boston Scientific Corporation), or implanted by a change in temperature (e.g., nitinol stent). Self-expanding stents that may be used include the coronary WALLSTENT and the SCIMED RADIUS stent from Boston Scientific Corporation (Natick, Mass.) and the GIANTURCO stents from Cook Group, Inc. (Bloomington, Ind.). Examples of balloon expandable stents that may be used include the CROSSFLEX stent, BX-VELOCITY stent and the PALMAZ-SCHATZ crown and spiral stents from Cordis Corporation (Miami Lakes, Fla.), the V-FLEX PLUS stent by Cook Group, Inc., the NIR, EXPRESS and LIBRERTE stents from Boston Scientific Corporation, the ACS MULTILINK, MULTILINK PENTA, SPIRIT, and CHAMPION stents from Guidant Corporation, and the Coronary Stent S670 and S7 by Medtronic, Inc. (Minneapolis, Minn.).

Other examples of stents that may have the subject polymer composition infiltrated into adjacent tissue in accordance with the invention include those from Boston Scientific Corporation, (e.g., the drug-eluting TAXUS EXPRESS² Paclitaxel-Eluting Coronary Stent System; over the wire stent stents such as the Express² Coronary Stent System and NIR Elite OTW Stent System; rapid exchange stents such as the EXPRESS² Coronary Stent System and the NIR ELITE MONORAIL Stent System; and self-expanding stents such as the MAGIC WALLSTENT Stent System and RADIUS Self Expanding Stent); Medtronic, Inc. (Minneapolis, Minn.) (e.g., DRIVER ABT578-eluting stent, DRIVER ZIPPER MX Multi-Exchange Coronary Stent System and the DRIVER Over-the-Wire Coronary Stent System; the S7 ZIPPER MX Multi-Exchange Coronary Stent System; S7, S670, S660, and BESTENT2 with Discrete Technology Over-the-Wire Coronary Stent System); Guidant Corporation (e.g., cobalt chromium stents such as the MULTI-LINK VISION Coronary Stent System; MULTI-LINK ZETA Coronary Stent System; MULTI-LINK PIXEL Coronary Stent System; MULTI-LINK ULTRA Coronary Stent System; and the MULTI-LINK FRONTIER); Johnson & Johnson/Cordis Corporation (e.g., CYPHER sirolimus-eluting Stent; PALMAZ-SCHATZ Balloon Expandable Stent; and S.M.A.R.T. Stents); Abbott Vascular (Redwood City, Calif.) (e.g., MATRIX LO Stent; TRIMAXX Stent; and DEXAMET stent); Conor Medsystems (Menlo Park, Calif.) (e.g., MEDSTENT and COSTAR stent); AMG GmbH (Germany) (e.g., PICO Elite stent); Biosensors International (Singapore) (e.g., MATRIX stent, CHAMPION Stent (formerly the S-STENT), and CHALLENGE Stent); Biotronik (Switzerland) (e.g., MAGIC AMS stent); Clearstream Technologies (Ireland) (e.g., CLEARFLEX stent); Cook Inc. (Bloomington, Ind.) (e.g., V-FLEX PLUS stent, ZILVER PTX self-expanding vascular stent coating, LOGIX PTX stent (in development); Devax (e.g., AXXESS stent) (Irvine, Calif.); DISA Vascular (Pty) Ltd (South Africa) (e.g., CHROMOFLEX Stent, S-FLEX Stent, S-FLEX Micro Stent, and TAXOCHROME DES); Intek Technology (Baar, Switzerland) (e.g., APOLLO stent); Orbus Medical Technologies (Hoevelaken, The Netherlands) (e.g., GENOUS); Sorin Biomedica (Saluggia, Italy) (e.g., JANUS and CARBOSTENT); and stents from Bard/Angiomed GmbH Medizintechnik K G (Murray Hill, N.J.), and Blue Medical Supply & Equipment (Mariettta, Ga.), Aachen Resonance GmbH (Germany); Eucatech A G (Germany), Eurocor GmbH (Bonn, Gemany), Prot, Goodman, Terumo (Japan), Translumina GmbH (Germany), MIV Therapeutics (Canada), Occam International B.V. (Eindhoven, The Netherlands), Sahajanand Medical Technologies PVT LTD. (India); AVI Biopharma/Medtronic/Interventional Technologies (Portland, OR) (e.g., RESTEN NG-coated stent); and Jomed (e.g., FLEXMASTER drug-eluting stent) (Sweden).

Generally, stents are inserted in a similar fashion regardless of the site or the disease being treated. Briefly, a preinsertion examination, usually a diagnostic imaging procedure, endoscopy, or direct visualization at the time of surgery, is generally first performed in order to determine the appropriate positioning for stent insertion. A guidewire is then advanced through the lesion or proposed site of insertion, and over this is passed a delivery catheter which allows a stent in its collapsed form to be inserted. Intravascular stents may be inserted into an artery such as the femoral artery in the groin and advanced through the circulation under radiological guidance until they reach the anatomical location of the plaque in the coronary or peripheral circulation. Typically, stents are capable of being compressed, so that they can be inserted through tiny cavities via small catheters, and then expanded to a larger diameter once they are at the desired location. The delivery catheter then is removed, leaving the stent standing on its own as a scaffold. Once expanded, the stent physically forces the walls of the passageway apart and holds them open. A post insertion examination, usually an x-ray, is often utilized to confirm appropriate positioning.

Stents are typically maneuvered into place under, radiologic or direct visual control, taking particular care to place the stent precisely within the vessel being treated. In certain aspects, the stent may further include a radio-opaque, echogenic material, or MRI responsive material (e.g., MRI contrast agent) to aid in visualization of the device under ultrasound, fluoroscopy and/or magnetic resonance imaging. The radio-opaque or MRI visible material may be in the form of one or more markers (e.g., bands of material that are disposed on either end of the stent) that may be used to orient and guide the device during the implantation procedure.

In another aspect, the subject polymer compositions may be infiltrated into tissue adjacent to an anastomotic connector device.

“Anastomotic connector device” refers to any vascular device that mechanizes the creation of a vascular anastomosis (e.g., artery-to-artery, vein-to-artery, artery-to-vein, artery-to-synthetic graft, synthetic graft-to-artery, vein-to-synthetic graft or synthetic graft-to-vein anastomosis) without the manual suturing that is typically done in the creation of an anastomosis. The term also refers to anastomotic connector devices (described below), designed to produce a facilitated semiautomatic vascular anastomosis without the use of suture and reduce connection time substantially (often to several seconds), where there are numerous types and designs of such devices. The term also refers to devices which facilitate attachment of a vascular graft to an aperture or orifice (e.g., in the side or at the end of a vessel) in a target vessel. Anastomotic connector devices may be anchored to the outside of a blood vessel, and/or into the wall of a blood vessel (e.g., into the adventitial, intramural, or intimal layer of the tissue), and/or a portion of the device may reside within the lumen of the vessel.

Anastomotic connector devices also may be used to create new flow from one structure to another through a channel or diversionary shunt. Accordingly, such devices (also referred to herein as “bypass devices”) typically include at least one tubular structure, wherein a tubular structure defines a lumen. Anastomotic connector devices may include one tubular structure or a plurality of tubular structures through which blood can flow. At least a portion of the tubular structure resides external to a blood vessel (e.g., extravascular) to provide a diversionary passageway. A portion of the device also may reside within the lumen and/or within the tissue of the blood vessel.

Examples of anastomotic connector devices are described in co-pending application entitled, “Anastomotic Connector Devices”, filed May 24, 2004 (U.S. Ser. No. 10/853,023). Representative examples of anastomotic connector devices include, without limitation, vascular clips, vascular sutures, vascular staples, vascular clamps, suturing devices, anastomotic coupling devices (e.g., anastomotic couplers), including couplers that include tubular segments for carrying blood, anastomotic rings, and percutaneous in situ coronary artery bypass (PISCAB and PICVA) devices. Broadly, anastomotic connector devices may be classified into three categories: (1) automated and modified suturing methods and devices, (2) micromechanical devices, and (3) anastomotic coupling devices.

(1) Automated and Modified Suturing Methods and Devices

Automated sutures and modified suturing methods generally facilitate the rapid deployment of multiple sutures, usually in a single step, and eliminate the need for knot tying or the use of aortic side-biting clamps. Suturing devices include those devices that are adapted to be minimally invasive such that anastomoses are formed between vascular conduits and hollow organ structures by applying sutures or other surgical fasteners through device ports or other small openings. With these devices, sutures and other fasteners are applied in a relatively quick and automated manner within bodily areas that have limited access. By using minimally invasive means for establishing anastomoses, there is less blood loss and there is no need to temporarily stop the flow of blood distal to the operating site. For example, the suturing device may be composed of a shaft-supported vascular conduit that is adapted for anastomosis and a collar that is slideable on the shaft configured to hold a plurality of needles and sutures that passes through the vascular conduit. See, e.g., U.S. Pat. No. 6,709,441. The suturing device may be composed of a carrier portion for inserting graft arm portions that extend to support the graft into position, and a needle assembly adapted to retain and advance coil fasteners into engagement with the vessel wall and the graft flange to complete the anastomosis. See, e.g., U.S. Pat. No. 6,709,442. The suturing device may include two oblong interlinked members that include a split bush adapted for suturing (e.g., U.S. Pat. No. 4,350,160).

One representative example of a suturing device is the HEARTFLOW device, made by Perclose-Abbott Labs, Redwood City, Calif. (see generally, U.S. Pat. Nos. 6,358,258, 6,355,050, 6,190,396, and 6,036,699, and PCT Publication No. WO 01/19257).

The nitinol U-CLIP suture clip device by Coalescent Surgical (Sunnyvale, Calif.) consists of a self-closing nitinol wire loop attached to a flexible member and a needle with a quick release mechanism. This device facilitates the construction of anastomosis by simplifying suture management and eliminating knot tying (see generally, U.S. Pat. Nos. 6,074,401 and 6,149,658, and PCT Publication Nos. WO 99/62406, WO 99/62409, WO 00/59380, WO 01/17441).

The ENCLOSE Anastomotic Assist Device (Novare Surgical Systems, Cupertino, Calif.) allows a surgeon to create a sutured anastomosis using standard suturing techniques but without the use of a partial occluding side-biting aortic clamp, avoiding aortic wall distortion (see U.S. Pat. Nos. 6,312,445 and 6,165,186).

In one aspect, automated and modified suturing methods and devices may deliver a surgical fastener (e.g., a suture or suture clip) suitable for having the subject polymer composition infiltrated into adjacent tissue. In another aspect, automated and modified suturing methods and devices may deliver a vascular graft that has the subject polymer composition infiltrated into adjacent tissue to complete an anastomosis.

(2) Micromechanical Devices

Micromechanical devices are used to create an anastomosis and/or secure a graft vessel to the site of an anastomosis. Representative examples of micromechanical devices include staples (either penetrating or non-penetrating) and clips.

Anastomotic staple and clip devices may take a variety of forms and may be made from different types of materials. For example, staples and clips may be formed of a metal or metal alloy, such as titanium, nickel-titanium alloy, or stainless steel, or a polymeric material, such as silicone, poly(urethane), rubber, or a thermoplastic elastomer.

The polymeric material may be an absorbable or biodegradable material designed to dissolve after completion of the anastomosis. Biodegradable polymers include, for example, homopolymers and copolymers that comprise one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one.

A variety of devices for guiding staples and clips into position also have been described.

One manufacturer of non-penetrating staples for use in the creation of anastomosis is United States Surgical Corp. (Norwalk, Conn.). The VCS system (Autosuture) is an automatic stapling device that applies non-penetrating, titanium vascular clips which are usually used in an interrupted fashion to evert tissue edges with high compressive forces. (See, e.g., U.S. Pat. Nos. 6,440,146, 6,391,039, 6,024,748, 5,833,698, 5,799,857, 5,779,718, 5,725,538, 5,725,537, 5,720,756, 5,360,154, 5,193,731, and 5,005,749 for the description of anastomotic connector devices made by U.S. Surgical).

An anastomotic clip may be composed of a shape memory material, such as nitinol, which is self-closing between an open U-shaped configuration and a closed configuration. See, e.g., U.S. Pat. No. 6,641,593. The anastomotic clip may be composed of a wire having a shape memory that defines a closed configuration which may be substantially spiral-shaped and having a needle that may be releasably attached to the clip. See, e.g., U.S. Pat. No. 6,551,332. Other anastomotic clips are described in, e.g., U.S. Pat. Nos. 6,461,365; and 6,514,265.

Automatic stapling devices are also made by Bypass/Ethicon, Inc. (Somerville, N.J.) and are described in, e.g., U.S. Pat. Nos. 6,193,129; 5,632,433; 5,609,285; 5,533,661; 5,439,156; 5,350,104; 5,333,773; 5,312,024; 5,292,053; 5,285,945; 5,275,322; 5,271,544; 5,271,543 and 5,205,459 and WO 03/02016. Resorbable surgical staples that include a polymer blend that is rich in glycolide (i.e., 65 to 85 weight % polymerized glycolide) are described in, e.g., U.S. Pat. Nos. 4,741,337 and 4,889,119. Surgical staples made from a blend of lactide/glycolide-copolymer and poly(p-dioxanone) are described in U.S. Pat. No. 4,646,741. Other types of stapling devices are described in, e.g., U.S. Pat. Nos. 5,234,447; 5,904,697 and 6,565,582; and U.S. Publication No. 2002/0185517A1.

In another aspect, the micromechanical device may be an anastomotic clip. For example, an anastomotic clip may be composed of a shape memory material, such as nitinol, which is self-closing between an open U-shaped configuration and a closed configuration. See, e.g., U.S. Pat. No. 6,641,593. The anastomotic clip may be composed of a wire having a shape memory that defines a closed configuration which may be substantially spiral-shaped and having a needle that may be releasably attached to the clip. See, e.g., U.S. Pat. No. 6,551,332. Other anastomotic clips are described in, e.g., U.S. Pat. Nos. 6,461,365; 6,187,019; and 6,514,265.

In one aspect, the present invention provides for of a micromechanical anastomotic device (e.g., a staple or a clip) having the subject polymer composition infiltrated into adjacent tissue.

(3) Anastomotic Coupling Devices

Anastomotic coupling devices-may be used to connect a first blood vessel to a second vessel, either with or without a graft vessel, for completion of an anastomosis. In one aspect, anastomotic coupling devices facilitate automated attachment of a graft or vessel to an aperture or orifice (e.g., in the side or at the end of a vessel) in a target vessel without the use of sutures or staples. In another aspect, the anastomotic coupling device comprises a tubular structure defining a lumen through which blood may flow (described below).

Anastomotic coupling devices that facilitate automated attachment of a graft or vessel to an aperture or orifice in a target vessel may take a variety of forms and may be made from a variety of materials. Typically, such devices are made of a biocompatible material, such as a polymer or a metal or metal alloy. For example, the device may be formed from a synthetic material, such as a fluoropolymer, such as expanded poly(tetrafluoroethylene) (ePTFE) sold under the trade name GORE-TEX available from W. L. Gore & Associates, Inc. or fluorinated ethylene propylene (FEP), a polyurethane, polyethylene, polyamide (nylon), silicone, polypropylene, polysulfone, or a polyester.

Anastomotic coupling devices may include an absorbable or biodegradable material designed to dissolve after completion of the anastomosis. Biodegradable polymers include, for example, homopolymers and copolymers that comprise one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one.

The device may include a metal or metal alloy (e.g., nitinol, stainless steel, titanium, iron, nickel, nickel-titanium, cobalt, platinum, tungsten, tantalum, silver, gold, molybdenum, chromium, and chrome), or a combination of a metal and a polymer.

The device may be anchored to the outside of a vessel, within the tissue that surrounds the lumen of a blood vessel, and/or a portion of the device may reside within the lumen of the vessel.

In one aspect, the anastomotic coupler may be an artificially formed aperture connector that is placed in the side wall of the target vessel so that the tubular graft conduit may be extended from the target vessel. The connector may include a plurality of tissue-piercing members and retention fingers disposed in a concentric annular array which may be passed through the side wall of the tubular graft conduit for securing and retaining the graft to the connector in a fluid-tight configuration. See, e.g., U.S. Pat. Nos. 6,702,829 and 6,699,256.

In another aspect, the anastomotic coupler may be in the form of a frame. For example, the frame may be configured to be deformable and scissor-shaped such that spreading members are moveable to secure a graft vessel upon insertion into a target vessel. See, e.g., U.S. Pat. No. 6,179,849.

In another aspect, the anastomotic coupler may be a ring-like device that is used as an anastomotic interface between a lumen of a graft and an opening in a lumen of a target vessel. For example, the anastomotic ring may be composed of stainless steel alloy, titanium alloy, or cobalt alloy and have a flange with an expandable diameter. See, e.g., U.S. Pat. No. 6,699,257. Anastomosis rings are also described in, e.g., U.S. Pat. No. 6,248,117.

In another aspect, the anastomotic coupler is resorbable. Resorbable anastomotic coupling devices may include, for example, a polymeric blend that is rich in glycolide (i.e., 65 to 85 weight % polymerized glycolide) (see, e.g., U.S. Pat. Nos. 4,741,337 and 4,889,119) or a blend of lactide/glycolide-copolymer and poly(p-dioxanone) (see, e.g., U.S. Pat. No. 4,646,741).

In another aspect, the anastomotic coupler includes a bioabsorbable, elastomeric material. Representative examples of elastomeric materials for use in resorbable devices are described in, e.g., U.S. Pat. No. 5,468,253.

In another aspect, the anastomotic coupler may be used to connect a first blood vessel to a second vessel, either with or without a graft vessel. For example, the anastomotic coupler may be a device that serves to interconnect two vessels in a side-to-side anastomosis, such as when grafting two juxtaposed cardiac vessels. The anastomotic coupler may be configured as two partially opened cylindrical segments that are interconnected along the periphery by a flow opening whereby the device may be inserted in a minimally-invasive manner which then conforms to provide pressure against the interior wall when in the original configuration such that leakage is prevented. See, e.g., U.S. Pat. Nos. 6,464,709; 6,458,140 and 6,251,116 and U.S. application Publication No. 2003/0100920A1.

In another aspect, the anastomotic coupler may also be incorporated in the design of a vascular graft to eliminate the step of attaching the interface prior to deployment. For example, the anastomotic coupler may have a leading and rear petal for dilating the vessel opening during advancement, and a base which is configured for attachment to a graft while forming a seal with the opening of the vessel. See, e.g., U.S. Pat. No. 6,702,828.

In another aspect, the anastomotic coupler may be in the form of a frame. For example, the anastomotic coupler may be composed of a deformable, scissor-shaped frame with spreading members that is inserted into a target vessel. See, e.g., U.S. Pat. No. 6,179,849.

In another aspect, the anastomotic coupling device may include a graft that incorporates fixation mechanisms (e.g., a collet or a grommet) at its opposite ends and a heating element to create a thermal bond between the graft and a blood vessel (see, e.g., U.S. Pat. Nos. 6,652,544 and 6,293,955).

In another aspect, the anastomotic coupling device includes a compressible, expandable fitting for securing the ends of a bypass graft to two vessels. The fitting may be incorporated in the bypass graft design to eliminate the step of attaching the graft to the fitting prior to deployment (see, e.g., U.S. Pat. No. 6,494,889).

In another aspect, the anastomotic coupling device includes a pair of coupling disc members for joining two vessels in an end-to-end or end-to-side fashion. One of the members includes hook members, while the other member has receptor cavities aligned with the hooks for locking everted tissue of the vessels together (see, e.g., U.S. Pat. No. 4,523,592).

Representative examples of anastomotic connector devices of Bypass/Ethicon, Inc. are described in U.S. application Publication Nos. US2002/0082625A1 and 2003/0100910A1 and U.S. Pat. Nos. 6,036,703, 6,036,700, 6,015,416, and 5,346,501.

Other anastomotic coupling devices are those described in e.g., U.S. Pat. Nos. 6,036,702; 6,508,822; 6,599,303; 6,673,084, 5,695,504; 6,569,173; 4,931,057; 5,868,763; 4,624,257; 4,917,090; 4,917,091; 5,697,943; 5,562,690; 5,454,825; 5,447,514; 5,437,684; 5,376,098; 6,652,542; 6,551,334; and 6,726,694 and U.S. application Publication Nos. 2003/0120293A1 and 2004/0030348A1.

Anastomotic coupling devices may include proximal aortic connectors and distal coronary connectors. For example, aortic anastomotic connectors include devices such as the SYMMETRY Bypass Aortic Connector device made by St. Jude Medical, Inc. (Maple Grove, Minn.), which consists of an aortic cutter or hole punch assembly and a graft delivery system. The aortic hole punch is a cylindrical cutter with a barbed needle that provides an anchor and back pressure for the rotating cutter to core a round hole in the wall of the aorta. The graft delivery system is a radially expandable nitinol device that holds the vein graft with small hooks which pierce through vein graft wall. The graft is fixed to the aorta through use of an inner and outer ring of struts or flanges. This and other anastomotic connector devices by St. Jude are described in U.S. Pat. Nos. 6,309,416, 6,302,905, 6,152,937, and PCT Publication Nos. WO 00/27312 and WO 00/27311.

The CORLINK automated anastomotic connector device, which is produced by the CardioVations division of Ethicon, Inc. (Johnson & Johnson, Somerville, N.J.), uses a nitinol metal alloy fastener to connect the grafted vessel to the aorta. It consists of a central cylindrical body made of interconnected elliptical arches and two sets of several pins radiating from each end. The graft is loaded into a CORLINK insertion instrument and deployed to create an anastomosis in one step.

Further examples of anastomotic coupling devices include those made by Cardica (see U.S. Pat. Nos. 6,719,769; 6,419,681 and 6,537,287), Converge Medical (formerly Advanced Bypass Technologies), Onux Medical (see, e.g., PCT Publication No. WO 01/34037) and Ventrica, Menlo Park, Calif. (VENTRICA Magnetic Vascular Positioner) (see, e.g., U.S. Pat. Nos. 6,719,768; 6,517,558 and 6,352,543).

As described above, an anastomotic coupling device may comprise a tubular structure defining a lumen through which blood may flow. These types of devices (also referred to herein as “bypass devices”) can function as an artificial passageway or conduit for fluid communication between blood vessels and can be used to divert (i.e., shunt) blood from one part of a blood vessel (e.g., an artery) to another part of the same vessel, or to a second vessel (e.g., an artery or a vein) or to multiple vessels (e.g., a vein and an artery). In one aspect of the invention, the anastomotic device is a bypass device.

Bypass devices may be used in a variety of end-to-end and end-to-side anastomotic procedures. The bypass device may be placed into a patient where it is desired to create a pathway between two or more vascular structures, or between two different parts of the same vascular structure. For example, bypass devices may be used to create a passageway which allows blood to flow around a blood vessel, such as an artery (e.g., coronary artery, carotid artery, or artery supplying the lower limb), which has become damaged or completely or partially obstructed. Bypass devices may be used in coronary artery bypass surgery to shunt blood from an artery, such as the aorta, to a portion of a coronary artery downstream from an occlusion in the artery.

Certain types of anastomotic coupling devices are configured to join two abutting vessels. The device may further include a tubular segment to shunt blood to another vessel. These types of connectors are often used for end-to-end anastomosis if a vessel is severed or injured.

Bypass devices include at least one tubular structure having a first end and a second end, which defines a single lumen through which blood can flow, or may include more than one tubular structure, defining multiple lumens through which blood can flow. The tubular structure includes an extravascular portion and may, optionally, include an intravascular portion. The extravascular portion resides external to the adventitial tissue of a blood vessel, whereas the intravascular portion may reside within the vessel lumen or within the intimal, medial, and/or adventitial tissue.

The configuration of the tubular segment may take a variety of forms. For example, the tubular portion may be generally straight, bent or curved (e.g., L-shaped or helical), tapered, branched (e.g., bifurcated or trifurcated), or may include a network of conduits through which blood may flow. Generally, straight or bent devices have a single lumen through which blood may flow, while branched conduits (e.g., generally T-shaped and Y-shaped devices) and conduit networks (described below) have two or more lumens through which blood may flow. A tubular structure may be in the form, for example, of a hollow cylinder and may or may not include a support structure, such as a mesh or porous framework. Depending on the procedure, the device may be biodegradable or non-biodegradable; expandable or rigid; metal and/or polymeric; and/or may include a shape-memory material (e.g., nitinol). In certain aspects, the device may include a self-expanding stent structure.

Bypass devices typically are made of a biocompatible material. Any of the materials described above for other types of connectors may be used to make a bypass device, such as a synthetic or naturally-derived polymer, or a metal or metal alloy. For example, the device may be formed from a synthetic material, such as a fluoropolymer, such as expanded poly(tetrafluoroethylene) (ePTFE) or fluorinated ethylene propylene (FEP), a polyurethane, polyethylene, polyamide (nylon), silicone, polypropylene, polysulfone, or a polyester and/or a naturally derived material, such as collagen or a polysaccharide. The device may include a metal or metal alloy (e.g., nitinol, stainless steel, titanium, nickel, nickel-titanium, cobalt, platinum, iron, tungsten, tantalum, silver, gold, molybdenum, chromium and chrome), or a combination of a metal and a polymer. Other types of devices include a natural graft material (e.g., autologous vessel, homologous vessel, or xenograft), or a combination of a synthetic and a natural graft material. In another aspect, the bypass device may be formed of an absorbable or biodegradable material designed to dissolve after completion of the anastomosis (e.g., polylactide, polyglycolide, and copolymers of lactide and glycolide). In yet another aspect, demineralized bone may be used to provide a pliable tubular conduit (see, e.g., U.S. Pat. No. 6,290,718).

The tubular structure(s) include a proximal end that may be configured for attachment to a proximal blood vessel and a distal end configured for attachment to a distal blood vessel. As described above, an anastomosis may be described as being either “proximal” or “distal” depending on its location relative to the vascular obstruction. The “proximal” anastomosis may be formed in a proximal blood vessel, and the “distal” anastomosis may be formed in a distal blood vessel, which may the same vessel or a different vessel than the proximal vessel. The terms “distal” and “proximal” may also be used to describe the direction that blood flows through a tubular structure from one vessel into another vessel. For example, blood may flow from a proximal vessel (e.g., the aorta) into a distal vessel, such as a coronary artery to bypass an obstruction in the coronary artery.

The tubular structure may be attached directly to a proximal or distal blood vessel. Alternatively, the bypass device may further include a graft vessel or be configured to receive a graft vessel, which can be connected to the same or a different blood vessel for completion of the anastomosis. Representative examples of graft vessels include, for example, vascular grafts or grafts used in hemodialysis applications (e.g., AV graft, AV shunt, or AV graft).

In one aspect, a tubular anastomotic coupler includes a proximal end that is attached to a proximal vessel and a distal end that is used to attach a bypass graft. The bypass graft can be secured to the distal vessel to complete the anastomosis. The direction of blood flow can be from the proximal blood vessel and into the proximal end of the tubular structure. Blood can exit through the distal end of the tubular structure and into the graft vessel.

In another aspect, the tubular anastomotic coupler includes a proximal end that is attached to a graft vessel, which is secured to the proximal blood vessel, and a distal end that is configured for attachment to a distal blood vessel. The direction of blood flow can be from the proximal vessel into the graft vessel and into the proximal end of the tubular structure. Blood can exit through the distal end of the tubular structure and into the distal vessel.

Anastomotic bypass devices may be anchored to a blood vessel in a variety of ways and may be attached to a blood vessel for the formation of an anastomosis with or without the use of sutures. Bypass devices may be attached to the outside of a blood vessel, and/or a portion of the device may be implanted into a vessel. For example, a portion of the implanted device may reside within the lumen of the vessel (i.e., endoluminally), and/or a portion of the implanted device may reside intravascularly (i.e., within the intimal, intramural, and/or adventitial tissue of the blood vessel). In one aspect, at least one of the tubular structures, or a portion thereof, may be inserted into the end of a vessel or into the side of a blood vessel. The device may be secured directly to the vessel using, for example, a fastener, such as sutures, staples, or clips and/or an adhesive. Bypass devices may include an interface to secure the conduit to a target vessel without the use of sutures. The interface may include means, such as, for example, hooks, barbs, pins, clamps, or a flange or lip for coupling the device to the site of an anastomosis.

Representative examples of anastomotic coupling devices that include at least one tubular portion include, without limitation, devices used for end-to-end anastomosis procedures (e.g., anastomotic stents and anastomotic sleeves) and end-to-side anastomosis procedures (e.g., single-lumen and multi-lumen bypass devices).

In one aspect of the invention, the anastomotic coupling device comprises a single tubular portion that may by used as a shunt to divert blood from a source vessel to a graft vessel (e.g., in an end-to-side anastomosis procedure). In one aspect, an end of the tubular portion may be connected directly or indirectly to a target vessel, as described above. The opposite end of the tubular portion may be attached to a graft vessel, where the graft vessel may be secured to a target vessel to complete the anastomosis.

The tubular portion(s) may be straight or may have a curved or bent shape (e.g., L-shaped or helical) and may be oriented orthogonally or at an angle relative to the vessel to which it is connected. In one aspect, the conduit may be secured into the site by, for example, a fastener, such as staples, clamps, or hooks, or by adhesives, radiofrequency sealing, or by other methods known to those skilled in the art.

In one aspect, the anastomotic coupling device may be, for example, a tubular metal braided graft with suture rings welded at the distal end to provide a means for securing in place to the target vessel. See, e.g., U.S. Pat. No. 6,235,054. Other types of conduits that are secured into the site include, e.g., U.S. Pat. Nos. 4,368,736 and 4,366,819.

In certain types of single-lumen coupling devices, the conduit terminates in a flange that resides within the lumen of the vessel. For example, the conduit may have a tubular body with a connector which has a plurality of extensions and is configured for disposition annularly within the inside of a tubular vessel. See, e.g., U.S. Pat. No. 6,660,015. In other devices, the flange may be attached into or onto the surface of the adventitial tissue of the blood vessel.

Other types of single-lumen bypass devices are described, for example, in U.S. Pat. Nos. 6,241,743; 6,428,550; 6,241,743; 6,428,550; 5,904,697; 5,290,298; 6,007,576; 6,361,559; 6,648,901, 4,931,057 and U.S. application Publication Nos. 2004/0015180A1, 2003/0065344A1, and 2002/0116018A1.

In one aspect of the invention, the anastomotic coupling device comprises more than one lumen through which blood may travel. Multi-lumen bypass devices may include two or more tubular portions configured to interconnect multiple (two or more) blood vessels. Multi-lumen coupling devices may be used in a variety of anastomosis procedures. For example, such devices may be used in coronary artery bypass graft (CABG) surgery to divert blood from an occluded proximal vessel (e.g., an artery) into one or more target (i.e., distal) vessels (e.g., an artery or vein).

In one aspect, at least one tubular portion may by used as a shunt for diverting blood between a source vessel and a target vessel. In another aspect, the device may be configured as an interface for securing a graft vessel to a target vessel for completion of an anastomosis. Depending on the procedure, the tubular arms may be of equal length and diameter or of unequal length and diameter and may include a tubular portion(s) that is expandable and/or includes a shape-memory material (e.g., nitinol). Furthermore, the tubular portions may be made of the same material or a different material.

In one aspect, one or more ends of a tubular portion may be inserted into the end or into the side of one or more blood vessels. In other embodiments, one or more tubular portions of the device may reside within the lumen of a blood or graft vessel. The device, optionally, may be secured to the blood vessel using a fastener or an adhesive, or another approach known to those skilled in the art.

At least one arm of the multi-lumen connector may be attached to a graft vessel. The graft vessel may be a synthetic graft, such as an ePTFE or polyester graft, or natural graft material (e.g., autologous vessel, homologous vessel, or xenograft), or a combination of a synthetic and a natural graft material. In certain embodiments, a graft vessel may be attached to an end of a tubular portion of the device, and a second graft vessel may be attached to the opposite end of the same tubular portion or to the end of another tubular portion. The graft vessel(s) may be further attached to a target vessel(s) for the completion of the anastomosis.

In one aspect, the device may include three or more tubular arms that extend from a junction site. For example, the multi-lumen device may be generally T-shaped or Y-shaped (i.e., having two or three lumens, respectively). For example, the multi-lumen device may be a T-shaped tubular graft connector having a longitudinal member that extends into the target vessel and a second section that is exterior to the vessel which provides a connection to an alternate tubular structure. See, e.g., U.S. Pat. Nos. 6,152,945 and 5,972,017. Other multi-lumen devices are described in, (see, e.g., U.S. Pat. Nos. 6,152,945; 6,451,033; 5,755,778; 5,922,022; 6,293,965; 6,517,558 and 6,626,914 and U.S. Publication No. 2004/0015180A1).

In another aspect, the device may be a tube for bypassing blood flow directly from a portion of the heart (e.g., left ventricle) to a coronary artery. For example, the device may be a hollow tube that may be partially closable by a one-way valve in response to movement of the cardiac tissue during diastole while permitting blood flow during systole (see, e.g., U.S. Pat. No. 6,641,610). The device may be an elongated rigid shunt body composed of a diversion tube having two apertures in which one may be disposed within the cyocardium of the left ventricle and the other may be disposed within the coronary artery (see, e.g., WO 00/15146 and U.S. application Publication No. 2003/0055371A1). The device may be a valved, tubular apparatus that is L- or T-shaped which is adapted for insertion into the wall of the heart to provide blood communication from the heart to a coronary vessel (see, e.g., U.S. Pat. No. 6,123,682).

In another aspect, the device may include a network of interconnected tubular conduits. For example, the device may include two tubular portions that may be oriented generally axially or orthogonally relative to each other. See U.S. Pat. Nos. 6,241,761 and 6,241,764. Communication between the two tubular structures may be achieved through a flow channel which facilitates blood to flow between the bores of each tube.

In another aspect, the anastomotic coupling device is a resorbable device that may be configured with two or three termini which provide a vessel interface without the need for sutures and provides a fluid communication through an intersecting lumen, such as a bypass graft or alternate vessel. See, e.g., U.S. application Publication Nos. 2002/0052572A1 and PCT Publication No. WO 02/24114A2. An anastomotic connector may also be formed of a resorbable tubular structure configured to include snap-connectors or other components for securing it to the tissue as well as hemostasis inducing sealing rings to prevent blood leakage. See, e.g., U.S. Pat. Nos. 6,056,762. The anastomotic connector may be designed with three legs whereby two legs are adapted to be inserted within the continuous blood vessel in a contracted state and then enlarged to form a tight fit and the third leg is adapted for connecting and sealing with a third conduit. See, e.g., U.S. Pat. No. 6,019,788.

An example of a commercially available multi-lumen anastomotic coupling device is the SOLEM graft connector (made by Jomed, Sweden). This device, which is described in more detail in PCT Publication No. WO 01/13820, and U.S. Pat. Nos. 6,179,848, D438618 and D429334, includes a T-shaped connector composed of nitinol and an ePTFE graft for completion of a distal anastomosis.

Another example of an anastomotic connector is the HOLLY GRAFT System (in development) for use in bypass surgery from CABG Medical, Inc. (Minneapolis, Minn.), which is described, e.g., in U.S. Pat. Nos. 6,241,761 and 6,241,764.

In one aspect, the present invention provides for an anastomotic coupling device having the subject polymer composition infiltrated into adjacent tissue. In one aspect, the anastomotic coupling device may be attached to a blood vessel for the formation of an anastomosis without the use of sutures or staples. In certain aspects, the anastomotic coupling device may comprise a tubular structure defining a lumen through which blood may flow, and an anti-scarring agent. The device may include one, two, three, or more lumens defined by one, two, three, or more tubular structures, depending on the number of vessels to be connected.

Introduction of an intravascular device into or onto an intramural, luminal, or adventitial portion of a blood vessel may irritate or damage the endothelial tissue of the blood vessel and/or may alter the natural hemodynamic flow through the vessel and/or may introduce or promote infection in and around the intravascular device. This irritation or damage may stimulate a cascade of biological events resulting in a fibrotic response, which can lead to the formation of scar tissue in the vessel, and/or resulting in an increased susceptibility to infection. Infiltration of the subject polymer compositions (either alone or containing an anti-scarring agent and/or anti-infective agent) in accordance with the invention into tissue adjacent to the device, or a portion of the device that is in direct contact with the blood vessel (e.g., a terminal portion or edge of the device), may inhibit one or more of the scarring processes described above (e.g., smooth muscle cell proliferation, cell migration, inflammation), making the vessel less prone to the formation of intimal hyperplasia and stenosis and/or may inhibit or prevent infection in and around the anastomotic connector.

Thus, in one aspect, the subject polymer compositions may be associated only with the portion of the intravascular device that is in contact with the blood or endothelial tissue. For example, the anti-scarring agent may be incorporated onto tissue adjacent to all or a portion of the intravascular portion of the device. In another aspect, the subject polymer compositions may be infiltrated into tissue adjacent to all or a portion of an extravascular portion of the device.

In another aspect, the subject polymer compositions may be infiltrated into tissue adjacent to a portion of or the entire surface of the device. In another aspect, the subject polymer composition is associated (e.g., infiltrated into adjacent tissue) with an anchoring member (e.g., a fastener, such as a staple or clip) that secures the device to a blood vessel.

As described above, anastomotic connector devices may include a polymer composition containing a fibrosis-inhibiting or anti-infective agent as a means to improve the clinical efficacy of the device. In another approach, the fibrosis-inhibiting and/or anti-infective agent may be incorporated into or onto a film or mesh (described in further detail below) that is applied in a perivascular manner to an anastomotic site (e.g., at the junction of a graft vessel and the blood vessel). These films or wraps may be used with any of the anastomotic connector devices described above and, typically, are placed around the outside of the anastomosis at the time of surgery. In other embodiments, the agent may be delivered to the anastomotic site in the form of a spray, paste, gel, or the like. In yet another approach, the agent may be infiltrated into the tissue adjacent to the graft vessel that is secured to the blood vessel with the connector device.

In yet another aspect, the subject polymer compositions may infiltrated into tissue adjacent to other specialized intravascular devices, such as coronary drug infusion guidewires, such as those available from TherOx, Inc., grafts and balloon over stent devices, such as are described in Wilensky, R. L. (1993) J. Am. Coll. Cardiol.: 21: 185A.

As described above, the present invention provides polymeric compositions that may be infiltrated into the tissue adjacent to the intravascular devices (e.g., anastomotic connectors, stents, drug-delivery balloons, intravascular catheters), where the polymeric composition may include a therapeutic agent (e.g., an anti-scarring or anti-infective agent). Numerous polymeric compositions for use with intravascular devices have been described above which-may be infiltrated into the tissue adjacent to the device (preferably near the device-tissue interface).

Polymeric compositions may be infiltrated around implanted intravascular devices by applying the composition directly and/or indirectly into and/or onto (a) tissue adjacent to the intravascular device; (b) the vicinity of the intravascular device-tissue interface; (c) the region around the intravascular device; and (d) tissue surrounding the intravascular device. Methods for infiltrating the subject polymer compositions into tissue adjacent to an intravascular device include delivering the polymer composition: (a) to the intravascular device surface (e.g., as an injectable, paste, gel or mesh) during the implantation procedure; (b) to the surface of the tissue (e.g., as an injectable, paste, gel, in situ forming gel or mesh) immediately prior to, or during, implantation of the intravascular device; (c) to the surface of the intravascular device and/or the tissue surrounding the implanted intravascular device (e.g., as an injectable, paste, gel, in situ forming gel or mesh) immediately after the implantation of the intravascular device; (d) by topical application of the composition into the anatomical space where the intravascular device may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the intravascular device may be inserted); (e) via percutaneous injection into the tissue surrounding the intravascular device as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device.

According to one aspect, any fibrosis-inhibiting and/or anti-infective agent described above may be utilized in the practice of the present invention. In one aspect of the invention, the subject polymeric compositions infiltrated into tissue adjacent to intravascular devices may contain a fibrosis-inhibiting agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As intravascular devices are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

Gastrointestinal Stents

In one aspect, the subject polymer compositions may be infiltrated into tissue adjacent to a gastrointestinal (GI) stent. The subject polymer compositions may contain a therapeutic agent (e.g., an anti-scarring and/or anti-infective agent). The term “GI stent” refers to devices that are located in the gastrointestinal tract including the biliary duct, pancreatic duct, colon, and the esophagus. GI stents are or comprise scaffoldings that are used to treat endoluminal body passageways that have become blocked due to disease or damage, including malignancy or benign disease.

In one aspect, the GI stent may be an esophageal stent used to keep the esophagus open whereby food is able to travel from the mouth to the stomach. For example, the esophageal stent may be composed of a cylindrical supporting mesh inner layer, retaining mesh outer layer and a semi-permeable membrane sandwiched between. See, e.g., U.S. Pat. No. 6,146,416. The esophageal stent may be a radially, self-expanding stent of open weave construction with an elastomeric film formed along the stent to prevent tissue ingrowth and distal cuffs that resist stent migration. See, e.g., U.S. Pat. No. 5,876,448. The esophageal stent may be composed of a flexible wire configuration to form a cylindrical tube with a deformed end portion increased to a larger diameter for anchoring pressure. See, e.g., U.S. Pat. No. 5,876,445. The esophageal stent may be a flexible, self-expandable tubular wall incorporating at least one truncated conical segment along the longitudinal axis. See, e.g., U.S. Pat. No. 6,533,810.

In another aspect, the GI stent may be a biliary stent used to keep the biliary duct open whereby bile is able to drain into the small intestines. For example, the biliary stent may be composed of shape memory alloy. See, e.g., U.S. Pat. No. 5,466,242. The biliary stent may be a plurality of radially extending wings with grooves which project from a helical core. See, e.g., U.S. Pat. Nos. 5,776,160 and 5,486,191.

In another aspect, the GI stent may be a colonic stent. For example, the colonic stent may be a hollow tubular body that may expand radially and be secured to the inner wall of the organ in a release fitting. See, e.g., European Patent Application No. EP1092400A2.

In another aspect, the GI stent may be a pancreatic stent used to keep the pancreatic duct open to facilitate secretion into the small intestines. For example, the pancreatic stent may be composed of a soft biocompatible material which is resiliently compliant which conforms to the duct's curvature and contains perforations that facilitates drainage. See, e.g., U.S. Pat. No. 6,132,471.

GI stents, which may benefit from having the subject polymer composition infiltrated into adjacent tissue according to the present invention, include commercially available products, such as the NIR Biliary Stent System and the WALLSTENT Endoprostheses from Boston Scientific Corporation.

In one aspect, the present invention provides GI stents having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with GI stents have been described above.

Polymeric compositions may be infiltrated around implanted GI stents by applying the composition directly and/or indirectly into and/or onto (a) tissue adjacent to the GI stent; (b) the vicinity of the GI stent-tissue interface; (c) the region around the GI stent; and (d) tissue surrounding the GI stent. Methods for infiltrating the subject polymer compositions into tissue adjacent to a GI stent include delivering the polymer composition: (a) to the GI stent surface (e.g., as an injectable, paste, gel or mesh) during the implantation procedure; (b) to the surface of the tissue (e.g., as an injectable, paste, gel, in situ forming gel or mesh) immediately prior to, or during, implantation of the GI stent; (c) to the surface of the GI stent and/or the tissue surrounding the implanted GI stent (e.g., as an injectable, paste, gel, in situ forming gel or mesh) immediately after the implantation of the GI stent; (d) by topical application of the composition into the anatomical space where the GI stent may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the GI stent as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device.

According to one aspect, any fibrosis-inhibiting and/or anti-infective agent described above may be utilized in the practice of the present invention. In one aspect of the invention, the subject polymer compositions infiltrated into tissue adjacent to GI stents may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As GI stents are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

Tracheal and Bronchial Stents

The present invention provides for infiltration of the subject polymer compositions into tissue adjacent to a tracheal or bronchial stent device. The subject polymer compositions may contain a therapeutic agent (e.g., an anti-scarring and/or anti-infective agent).

Representative examples of tracheal or bronchial stents that may benefit from having the subject polymer compositions infiltrated into adjacent tissue include tracheal stents or bronchial stents, including metallic and polymeric tracheal or bronchial stents and tracheal or bronchial stents that have an external covering (e.g., polyurethane, poly(ethylene terephthalate), PTFE, or silicone rubber).

Tracheal and bronchial stents may be, for example, composed of an elastic plastic shaft with metal clasps that expands to form a lumen along the axis for opening the diseased portion of the trachea and having three sections to emulate the natural shape of the trachea. See, e.g., U.S. Pat. No. 5,480,431. The tracheal/bronchial stent may be a T-shaped tube having a tracheotomy tubular portion that projects outwardly through a tracheotomy orifice which is configured to close and form a fluid seal. See, e.g., U.S. Pat. Nos. 5,184,610 and 3,721,233. The tracheal/bronchial stent may be composed of a flexible, synthetic polymeric resin with a tracheotomy tube mounted on the wall with a bifurcated bronchial end that is configured in a T-Y shape with specific curves at the intersections to minimize tissue damage. See, e.g., U.S. Pat. No. 4,795,465. The tracheal/bronchial stent may be a scaffolding configured to be substantially cylindrical with a shape-memory frame having geometrical patterns and having a coating of sufficient thickness to prevent epithelialization. See, e.g., U.S. patent application Publication No. 2003/0024534A1.

Tracheal/bronchial stents, which may benefit from having the subject polymer composition infiltrated into adjacent tissue according to the present invention, include commercially available products, such as the WALLSTENT Tracheobronchial Endoprostheses and ULTRAFLEX Tracheobronchial Stent Systems from Boston Scientific Corporation and the DUMON Tracheobronchial Silicone Stents from Bryan Corporation (Woburn, Mass.).

In one aspect, the present invention provides tracheal and bronchial stents having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in tracheal and bronchial stents have been described above.

Polymeric compositions may be infiltrated around implanted tracheal and bronchial stents by applying the composition directly and/or indirectly into and/or onto (a) tissue adjacent to the tracheal/bronchial stent; (b) the vicinity of the tracheal/bronchial stent-tissue interface; (c) the region around the tracheal/bronchial stent; and (d) tissue surrounding the tracheal/bronchial stent. Methods for infiltrating the subject polymer compositions into tissue adjacent to a tracheal/bronchial stent include delivering the polymer composition: (a) to the tracheal/bronchial stent surface (e.g., as an injectable, paste, gel or mesh) during the implantation procedure; (b) to the surface of the tissue (e.g., as an injectable, paste, gel, in situ forming gel or mesh) immediately prior to, or during, implantation of the tracheal/bronchial stent; (c) to the surface of the tracheal/bronchial stent and/or the tissue surrounding the implanted tracheal/bronchial stent (e.g., as an injectable, paste, gel, in situ forming gel or mesh) immediately after the implantation of the tracheal/bronchial stent; (d) by topical application of the composition into the anatomical space where the tracheal/bronchial stent may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the tracheal/bronchial stent as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device.

According to one aspect, any fibrosis-inhibiting and/or anti-infective agent described above may be utilized in the practice of the present invention. In one aspect of the invention, the subject polymer compositions infiltrated into tissue adjacent to tracheal and bronchial stents may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As tracheal and bronchial stents are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ M to 10⁻⁷ M, or about 10⁻⁷ M to 10−6 M about 10⁻⁶ M to 10⁻⁵ M or about 10⁻⁵ M to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

Genital-Urinary Stents

In one aspect, the subject polymer compositions may be infiltrated into tissue adjacent to a genital-urinary (GU) stent device. The subject polymer compositions may contain a therapeutic agent (e.g., an anti-scarring and/or anti-infective agent).

Representative examples genital-urinary (GU) stents that may benefit from having the subject polymer compositions infiltrated into adjacent tissue include ureteric and urethral stents, fallopian tube stents, prostate stents, including metallic and polymeric GU stents and GU stents that have an external covering (e.g., polyurethane, poly(ethylene terephthalate), PTFE or silicone rubber).

In one aspect, genital-urinary stents include ureteric and urethral stents. Ureteral stents are hollow tubes with holes along the sides and coils at either end to prevent migration. Ureteral stents are used to relieve obstructions (caused by stones or malignancy), to facilitate the passage of stones, or to allow healing of ureteral anastomoses or leaks following surgery or trauma. They are placed endoscopically via the bladder or percutaneously via the kidney.

Urethral stents are used for the treatment of recurrent urethral strictures, detruso-external sphincter dyssynergia and bladder outlet obstruction due to benign prostatic hypertrophy. In addition, procedures that are conducted for the prostate, such as external radiation or brachytherapy, may lead to fibrosis and/or infection due to tissue insult resulting from these procedures. The incidence of urethral stricture in prostate cancer patients treated with external beam radiation is about 2%. Development of urethral stricture may also occur in other conditions such as following urinary catheterization or surgery, which results in damage to the epithelium of the urethra. The clinical manifestation of urinary tract obstruction includes decreased force and caliber of the urinary stream, intermittency, postvoid dribbling, hesitance and nocturia. Complete closure of the urethra can result in numerous problems including eventual kidney failure. To maintain patency in the urethra, urethral stents may be used. The stents are typically self-expanding and composed of metal superalloy, titanium, stainless steel or polyurethane.

For example, the ureteric/urethral stent may be composed of a main catheter body of flexible polymeric material having an enlarged entry end with a hydrophilic tip that dissolves when contacted with body fluids. See, e.g., U.S. Pat. No. 5,401,257. The ureteric/urethral stent may be composed of a multi-sections including a closed section at that the bladder end which does not contain any fluid passageways such that it acts as an anti-reflux device to prevent reflux of urine back into the kidney. See, e.g., U.S. Pat. No. 5,647,843. The ureteric/urethral stent may be composed of a central catheter tube made of shape memory material that forms a stent with a retention coil for anchoring to the ureter. See, e.g., U.S. Pat. No. 5,681,274. The ureteric/urethral stent may be a composed of an elongated flexible tubular stent with preformed set curls at both ends and an elongated tubular rigid extension attached to the distal end which allows the combination function as an externalized ureteral catheter. See, e.g., U.S. Pat. Nos. 5,221,253 and 5,116,309. The ureteric/urethral stent may be composed of an elongated member, a proximal retention structure, and a resilient portion connecting them together, whereby they are all in fluid communication with each other with a slideable portion providing a retracted and expanded position. See, e.g., U.S. Pat. No. 6,685,744. The ureteric/urethral stent may be a hollow cylindrical tube that has a flexible connecting means and locating means that expands and selectively contracts. See, e.g., U.S. Pat. No. 5,322,501. The ureteric/urethral stent may be composed of a stiff polymeric body that affords superior columnar and axial strength for advancement into the ureter, and a softer bladder coil portion for reducing the risk of irritation. See, e.g., U.S. Pat. No. 5,141,502. The ureteric/urethral stent may be composed of an elongated tubular segment that has a pliable wall at the proximal region and a plurality of members that prevent blockage of fluid drainage upon compression. See, e.g., U.S. Pat. No. 6,676,623. The ureteric/urethral stent may be a catheter composed of a conduit which is part of an assembly that allows for non-contaminated insertion into a urinary canal by providing a sealing member that surrounds the catheter during dismantling. See, e.g., U.S. patent application Publication No. 2003/0060807A1.

In another aspect, genital-urinary stents include prostatic stents. For example, the prostatic stent may be composed of two polymeric rings constructed of tubing with a plurality of connecting arm members connecting the rings in a parallel manner. See, e.g., U.S. Pat. No. 5,269,802. The prostatic stent may be composed of thermoplastic material and a circumferential reinforcing helical spring, which provides rigid mechanical support while being flexible to accommodate the natural anatomical bend of the prostatic urethra. See, e.g., U.S. Pat. No. 5,069,169.

In another aspect, genital-urinary stents include fallopian stents and other female genital-urinary devices. For example, the genital-urinary device may be a female urinary incontinence device composed of a vaginal-insertable supporting portion that is resilient and flexible, which is capable of self-support by expansion against the vaginal wall and extending about the urethral orifice. See, e.g., U.S. Pat. No. 3,661,155. The genital-urinary device may be a urinary evacuation device composed of a ovular bulbous concave wall having an opening to a body engaging perimetal edge integral with the wall and an attached tubular member with a pleated body. See, e.g., U.S. Pat. No. 6,041,448.

Genital-urinary stents, which may benefit from having the subject polymer composition infiltrated into adjacent tissue according to the present invention, include commercially available products, such as the UROLUME Endoprosthesis Stents from American Medical Systems, Inc. (Minnetonka, Minn.), the RELIEVE Prostatic/Urethral Endoscopic Device from InjecTx, Inc. (San Jose, Calif.), the PERCUFLEX Ureteral Stents from Boston Scientific Corporation, and the TARKINGTON Urethral Stents and FIRLIT-KLUGE Urethral Stents from Cook Group Inc (Bloomington, Ind.).

In one aspect, the present invention provides GU stents having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with GU stents have been described above.

Polymeric compositions may be infiltrated around implanted GU stents by applying the composition directly and/or indirectly into and/or onto (a) tissue adjacent to the GU stent; (b) the vicinity of the GU stent-tissue interface; (c) the region around the GU stent; and (d) tissue surrounding the GU stent. Methods for infiltrating the subject polymer compositions into tissue adjacent to a GU stent include delivering the polymer composition: (a) to the GU stent surface (e.g., as an injectable, paste, gel or mesh) during the implantation procedure; (b) to the surface of the tissue (e.g., as an injectable, paste, gel, in situ forming gel or mesh) immediately prior to, or during, implantation of the GU stent; (c) to the surface of the GU stent and/or the tissue surrounding the implanted GU stent (e.g., as an injectable, paste, gel, in situ forming gel or mesh) immediately after the implantation of the GU stent; (d) by topical application of the composition into the anatomical space where the GU stent may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the GU stent as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device.

According to one aspect, any fibrosis-inhibiting and/or anti-infective agent described above may be utilized in the practice of the present invention. In one aspect of the invention, the subject polymer compositions infiltrated into tissue adjacent to GU stents may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced. Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As GU stents are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

Ear and Nose Stents

In one aspect, the present subject polymer compositions may be infiltrated into tissue adjacent to an ear-nose-throat (ENT) stent device (e.g., a lacrimal duct stent, Eustachian tube stent, nasal stent, or sinus stent). The subject polymer compositions may contain a therapeutic agent (e.g., an anti-scarring and/or anti-infective agent).

The sinuses are four pairs of hollow regions contained in the bones of the skull named after the bones in which they are located (ethmoid, maxillary, frontal and sphenoid). All are lined by respiratory mucosa which is directly attached to the bone. Following an inflammatory insult such as an upper respiratory tract infection or allergic rhinitis, a purulent form of sinusitis can develop. Occasionally secretions can be retained in the sinus due to altered ciliary function or obstruction of the opening (ostea) that drains the sinus. Incomplete drainage makes the sinus prone to infection typically with Haemophilus influenza, Streptococcus pneumoniae; Moraxella catarrhalis, Veillonella, Peptococcus, Corynebacterium acnes and certain species of fungi.

When initial treatment such as antibiotics, intranasal steroid sprays and decongestants are ineffective, it may become necessary to perform surgical drainage of the infected sinus. Surgical therapy often involves debridement of the ostea to remove anatomic obstructions and removal of parts of the mucosa. Occasionally a stent (a cylindrical tube which physically holds the lumen of the ostea open) is left in the osta to ensure drainage is maintained even in the presence of postoperative swelling. ENT stents, typically made of stainless steel or plastic, remain in place for several days or several weeks before being removed.

Representative examples of ENT stents which may benefit from having the subject polymer composition infiltrated into adjacent tissue according to the present invention, include lacrimal duct stents, Eustachian tube stents, nasal stents, and sinus stents.

In one aspect, the present invention provides for a lacrimal duct stent having a polymer composition containing a therapeutic agent (e.g., an anti-scarring and/or anti-infective agent) infiltrated into adjacent tissue.

In another aspect, the present invention provides for a Eustachian tube stent having a polymer composition containing a therapeutic agent (e.g., an anti-scarring and/or anti-infective agent) infiltrated into adjacent tissue.

In yet another aspect, the present invention provides for a sinus stent having a polymer composition containing a therapeutic agent (e.g., an anti-scarring and/or anti-infective agent) infiltrated into adjacent tissue.

In yet another aspect, the present invention provides for a nasal stent having a polymer composition containing a therapeutic agent (e.g., an anti-scarring and/or anti-infective agent) infiltrated into adjacent tissue.

The ENT stent may be a choanal atresia stent composed of two long hollow tubes that are bridged by a flexible transverse tube. See, e.g., U.S. Pat. No. 6,606,995. The ENT stent may be an expandable nasal stent for postoperative nasal packing composed of a highly porous, pliable and absorbent foam material capable of expanding outwardly, which has a nonadherent surface. See, e.g., U.S. Pat. No. 5,336,163. The ENT stent may be a nasal stent composed of a deformable cylinder with a breathing passageway that has a smooth outer non-absorbent surface used for packing the nasal cavity following surgery. See, e.g., U.S. Pat. No. 5,601,594. The ENT stent may be a ventilation tube composed of a flexible, plastic, tubular vent with a rectangular flexible flange which is used for the nasal sinuses following endoscopic antrostomy. See, e.g., U.S. Pat. No. 5,246,455. The ENT stent may be a ventilating ear tube composed of a shaft and an extended tab which is used for equalizing the pressure between the middle ear and outer ear. See, e.g., U.S. Pat. No. 6,042,574. The ENT stent may be a middle ear vent tube composed of a non-compressible, tubular base and an eccentric flange. See, e.g., U.S. Pat. No. 5,047,053.

ENT stents, which may benefit from having the subject polymer composition infiltrated into adjacent tissue according to the present invention, include commercially available products such as Genzyme Corporation (Ridgefield, N.J.) SEPRAGEL Sinus Stents and MEROGEL Nasal Dressing and Sinus Stents from Medtronic Xomed Surgical Products, Inc. (Jacksonville, Fla.).

In one aspect, the present invention provides ENT stents having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with ENT stents have been described above.

Polymeric compositions may be infiltrated around implanted ENT stents by applying the composition directly and/or indirectly into and/or onto (a) tissue adjacent to the ENT stent; (b) the vicinity of the ENT stent-tissue interface; (c) the region around the ENT stent; and (d) tissue surrounding the ENT stent. Methods for infiltrating the subject polymer compositions into tissue adjacent to a ENT stent include delivering the polymer composition: (a) to the ENT stent surface (e.g., as an injectable, paste, gel or mesh) during the implantation procedure; (b) to the surface of the tissue (e.g., as an injectable, paste, gel, in situ forming gel or mesh) immediately prior to, or during, implantation of the ENT stent; (c) to the surface of the ENT stent and/or the tissue surrounding the implanted ENT stent (e.g., as an injectable, paste, gel, in situ forming gel or mesh) immediately after the implantation of the ENT stent; (d) by topical application of the composition into the anatomical space where the ENT stent may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the ENT stent as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device.

According to one aspect, any fibrosis-inhibiting and/or anti-infective agent described above may be utilized in the practice of the present invention. In one aspect of the invention, the subject polymer compositions infiltrated into tissue adjacent to ENT stents may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As ENT stents are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

Ear Ventilation Tubes

In another aspect, the subject polymer compositions may be infiltrated into tissue adjacent to an ear ventilation tube (also referred to as a tympanostomy tube). The subject polymer compositions may contain a therapeutic agent (e.g., an anti-scarring and/or anti-infective agent). Acute otitis media is the most common bacterial infection, the most frequent indication for surgical therapy, the leading cause of hearing loss and a common cause of impaired language development in children. The cost of treating this condition in children under the age of five is estimated at $5 billion annually in the United States alone. In fact, 85% of all children will have at least one episode of otitis media and 600,000 will require surgical therapy annually. The prevalence of otitis media is increasing and for severe cases surgical therapy is more cost effective than conservative management.

Acute otitis media (bacterial infection of the middle ear) is characterized by Eustachian tube dysfunction leading to failure of the middle ear clearance mechanism. The most common causes of otitis media are Streptococcus pneumoniae (30%), Haemophilus influenza (20%), Branhamella catarrhalis (12%), Streptococcus pyogenes (3%), and Staphylococcus aureus (1.5%). The end result is the accumulation of bacteria, white blood cells and fluid which, in the absence of an ability to drain through the Eustachian tube, results in increased pressure in the middle ear. For many cases antibiotic therapy is sufficient treatment and the condition resolves. However, for a significant number of patients the condition becomes frequently recurrent or does not resolve completely. In recurrent otitis media or chronic otitis media with effusion, there is a continuous build-up of fluid and bacteria that creates a pressure gradient across the tympanic membrane causing pain and impaired hearing. Fenestration of the tympanic membrane (typically with placement of a tympanostomy tube) relieves the pressure gradient and facilitates drainage of the middle ear (through the outer ear instead of through the Eustachian tube—a form of “Eustachian tube bypass”).

Recurrent otitis media or otitis media with effusion may be treated with tympanostomy tubes or artificial Eustachian tubes/stents, such as described above. These ventilation tubes are indicated for chronic otitis media with effusion, recurrent acute otitis media, tympanic membrane atelectasis, and complications of acute otitis media in children. The excessive formation of granulation tissue around these devices can result in a decreased functioning of these devices. This can then result in a second procedure to either clear the obstruction or to insert a new device. The incorporation of a fibrosis-inhibiting agent into or onto the ventilation tubes may prevent the overgrowth of this granulation tissue.

Surgical placement of tympanostomy tubes is the most widely used treatment for chronic otitis media because, although not curative, it improves hearing (which in turn improves language development) and reduces the incidence of acute otitis media. Tympanostomy tube placement is one of the most common surgical procedures in the United States with 1.3 million surgical placements per year.

Representative examples of ear ventilation tubes that may benefit from having the subject polymer composition infiltrated into adjacent tissue include, without limitation, grommet-shaped tubes, T-tubes, tympanostomy tubes, drain tubes, tympanic tubes, otological tubes, myringotomy tubes, artificial Eustachian tubes, Eustachian tube prostheses, and Eustachian stents. Ear ventilation tubes have been made out of, e.g., polytetrafluoroethylene (e.g., TEFLON), silicone, nylon, polyethylene and other polymers, stainless steel, titanium, and gold plated steel.

In one aspect, the ear ventilation tube may be a tympanostomy tube that is used to provide an alternative conduit for ventilation of the middle ear cavity via the external ear canal. Typically, ventilation of the middle ear is performed by conducting a myringotomy, in which a slit or opening in the tympanic membrane is surgically made to alleviate a buildup or reduction of pressure in the middle ear cavity and to drain accumulated fluids. Tympanostomy tubes may be inserted into the surgical slit of the tympanic membrane to serve as a bypass for the normal Eustachian tube, which drains the middle ear cavity under normal conditions. For example, the tympanostomy tube may be an elongated uniform tubular member composed of pure titanium or titanium alloy that has a concavity inwardly spaced from one end that forms a flange. See, e.g., U.S. Pat. No. 5,645,584. The tympanostomy tube may be composed of a micro-pitted titanium exterior flangeless surface used to ventilate the middle ear. See, e.g., U.S. Pat. No. 4,971,076. The tympanostomy tube may be composed of a shaft with a tab that extends outwardly perpendicular from the bottom of the shaft. See, e.g., U.S. Pat. No. 6,042,574. The tympanostomy tube may be a permanent ear ventilation device composed of an elongated tubular base having a flange eccentrically connected made of a non-compressible material. See, e.g., U.S. Pat. No. 5,047,053. The tympanostomy tube may be composed of a cap-plug, central body and end cap, which together form a plurality of lumens within the tube. See, e.g., U.S. Pat. No. 5,851,199. The tympanostomy tube may be composed of a microporous resin cured to form a gas-permeable matrix containing a homogenous dispersion of silver particles capable of migrating to the surface of the tube sidewalls to provide antimicrobial activity. See, e.g., U.S. Pat. No. 6,361,526. The tympanostomy tube may be composed of tubular body and a rib structure that projects outwardly to define a channel spiraling around the tubular body. See, e.g., U.S. Pat. No. 5,775,336. The tympanostomy tube may be composed of an integral cutting tang extending from one of two flanges of a grommet for incising the tympanic membrane. See, e.g., U.S. Pat. Nos. 5,827,295 and 5,643,280. The tympanostomy tube may be composed of a tubular member having two opposed flanges in which the insertion of the tube is facilitated by a cutting edge on the flange which induces an incision of the tympanic membrane. See, e.g., U.S. Pat. Nos. 5,489,286; 5,466,239; 5,254,120 and 5,207,685. Other tympanostomy tubes are described in, e.g., U.S. Pat. Nos. 6,406,453; 5,178,623; 4,808,171 and 4,744,792.

In another aspect, the ear ventilation tube may be used to establish the normal function of the Eustachian tube and thus, attempt to resolve the stenosis that prevents its normal function. Fluid in the middle ear cavity normally secretes away from the tympanic membrane and thus, restoring the normal function of the Eustachian tube may provide optimal ventilation and drainage. For example, the ventilation tube may be an Eustachian stent composed of a hollow tubular body having a compressible core with two connected parallel arms and a radially-oriented flange, which is placed in the Eustachian tube to maintain patency. See, e.g., U.S. Pat. No. 6,589,286. The ventilation tube may be an Eustachian tube prosthesis composed of a flexible tube having a flange that extends radially for positioning within the Eustachian tube passageway. See, e.g., U.S. Pat. No. 4,015,607.

Tympanostomy tubes, which may benefit from having the subject polymer composition infiltrated into adjacent tissue according to the present invention, include commercially available products. For example, Medtronic Xomed, Inc. (Jackonsville, Fla.) sells a variety of ear ventilation tubes, including Long-Term Ventilation Tubes and Grommet Style Ventilation Tubes, including ARMSTRONG Grommets, GOODE T-Grommets, VENTURI Style Ventilation Tubes, SHEEHY Type Collar Buttons, REUTER Bobbins, COHEN T-Grommets, and SOILEAU TYTAN Titanium Tubes. Micromedics, Inc. (Eagan, Minn.) also sells a variety of ear ventilation tubes, including BAXTER Bevel Buttons, TINY TOUMA, SPOONER, TOUMA T-Tubes, SHOEHORN Bobbins, SHAH, and SILVERSTEIN MICROWICK Eustachian Tubes. Gyrus ENT LLC (Bartlett, Tenn.) also sells a variety of ear ventilation tubes, including ULTRASIL Ventilation Tubes, RICHARDS COLLAR Bobbins, BALDWIN BUTTERFLY Ventilation Tubes and PAPARELLA 2000 Tubes.

In one aspect, the present invention provides ear ventilation tube devices having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with ear ventilation tube devices have been described above.

Polymeric compositions may be infiltrated around implanted ear ventilation tube devices by applying the composition directly and/or indirectly into and/or onto (a) tissue adjacent to the ear ventilation tube devices; (b) the vicinity of the ear ventilation tube device-tissue interface; (c) the region around the ear ventilation tube device; and (d) tissue surrounding the ear ventilation tube device. Methods for infiltrating the subject polymer compositions into tissue adjacent to an ear ventilation tube device include delivering the polymer composition: (a) to the ear ventilation tube device surface (e.g., as an injectable, paste, gel or mesh) during the implantation procedure; (b) to the surface of the tissue (e.g., as an injectable, paste, gel, in situ forming gel or mesh) immediately prior to, or during, implantation of the ear ventilation tube device; (c) to the surface of the ear ventilation tube device and/or the tissue surrounding the implanted ear ventilation tube device (e.g., as an injectable, paste, gel, in situ forming gel or mesh) immediately after the implantation of the ear ventilation tube device; (d) by topical application of the composition into the anatomical space where the ear ventilation tube device may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the ear ventilation tube device as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device.

According to one aspect, any fibrosis-inhibiting and/or anti-infective agent described above can be utilized in the practice of the present invention. In one aspect of the invention, the subject polymer compositions infiltrated into tissue adjacent to ear ventilation tubes may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As ear ventilation tubes are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

Intraocular Implants

In another aspect, the subject polymer compositions may be infiltrated into tissue adjacent to an intraocular implant. The subject polymer compositions may contain a therapeutic agent (e.g., an anti-scarring and/or anti-infective agent).

In one embodiment, the intraocular implant is an intraocular lens device for the prevention of lens (e.g., anterior or posterior lens) opacification. Eyesight deficiencies that may be treated with intraocular lenses include, without limitation, cataracts, myopia, hyperopia, astigmatism and other eye diseases. Intraocular lenses are most commonly used to replace the natural crystalline lens which is removed during cataract surgery. A cataract results from a change in the transparency of the normal crystalline lens in the eye. When the lens becomes opaque from calcification (e.g., yellow and/or cloudy), the light cannot enter the eye properly and vision is impaired.

Implantation of intraocular lenses into the eye is a standard technique to restore useful vision in diseased or damaged eyes. The number of intraocular lenses implanted in the United States has grown exponentially over the last decade. Currently, over 1 million intraocular lenses are implanted annually, with the vast majority (90%) being placed in the posterior chamber of the eye. The intent of intraocular lenses is to replace the natural crystalline lens (i.e., aphakic eye) or to supplement and correct refractive errors (i.e., phakic eye, natural crystalline lens is not removed).

Implanted intraocular lenses may develop complications caused by mechanical trauma, inflammation, infection or optical problems. Mechanical and inflammatory injury may lead to reduced vision, chronic pain, secondary cataracts, corneal decompensation, cystoid macular edema, hyphema, uveitis or glaucoma. One common problem that occurs with cataract extraction is opacification which results from the tissue's reaction to the surgical procedure or to the artificial lens. Opacification leads to clouding of the intraocular lens, thus reducing the long-term benefits. Opacification typically results when proliferation and migration of epithelial cells occur along the posterior capsule behind the intraocular lens. Subsequent surgery may be required to correct this reaction; however, it involves a complex technical process and may lead to further serious, sight-threatening complications. Therefore, coating or incorporating the intraocular lens with a fibrosis-inhibiting agent may reduce these complications.

Representative examples of intraocular lenses that may benefit from having the subject polymer composition infiltrated into adjacent tissue include, without limitation, polymethylmethacrylate (PMMA) intraocular lenses, silicone intraocular lenses, achromatic lenses, pseudophakos, phakic lenses, aphakic lenses, multi-focal intraocular lenses, hydrophilic and hydrophobic acrylic intraocular lenses, intraocular implants, optic lenses and rigid gas permeable (RGP) lenses.

In one aspect, intraocular lenses may be foldable or rigid. The foldable lenses may be inserted in a small incision site using a tiny tube whereas the hard lenses are inserted through a larger incision site. Foldable lenses may be composed of silicone, acrylic or hydrogel whereas rigid lenses may be composed of hard polymeric compositions (PMMA).

In one aspect, the intraocular lens may be used as an implant for the treatment of cataracts, where the natural crystalline lens of the eye has been removed (i.e., aphakic lens). For example, the intraocular lens may be composed of two lenses having distinct refractive indices and distinct optical powers being joined together as an achromatic lens that may be connected within a posterior or anterior chamber of the eye. See, e.g., U.S. Pat. No. 5,201,762. The intraocular lens may be secured in the posterior chamber by a system of posts that protrude through the iris attached to retaining rings. See, e.g., U.S. Pat. No. 4,053,953. The intraocular lens may be hard with a shape memory which is capable of deforming for insertion into the eye but will harden at normal body temperature. See, e.g., U.S. Pat. No. 4,946,470. The intraocular lens may be coated with proteins, polypeptides, polyamino acids, polyamines or carbohydrates bound to the surface of the implant. See, e.g., U.S. Pat. Nos. 6,454,802 and 6,106,554. Other examples of aphakic intraocular lenses are described in, e.g., U.S. Pat. Nos. 6,599,317; 6,585,768; 6,558,419; 6,533,813; 6,210,438; 5,266,074; 4,753,654; 4,718,904 and 4,704,123.

In another aspect, the intraocular lens may be used as a corrective implant for vision impairment, where the natural crystalline lens of the eye has not been removed (i.e., phakic lens). For example, the intraocular lens may be a narrow profile, glare reducing, phakic anterior chamber lens that may be composed of an optic zone and a transition zone that has a curvature shaped to minimize direct glare. See, e.g., U.S. Pat. No. 6,596,025. The intraocular lens may be a self-centering phakic lens inserted in the posterior chamber lens in which arms (i.e., haptic bodies) extend outwardly and protrude into the pupil such that the iris provides centering force to keep lens in place. See, e.g., U.S. Pat. No. 6,015,435. The intraocular lens may be composed of a circumferential edge and two haptics extending from the edge to a transverse member which is substantially straight or bowed inward toward the lens. See, e.g., U.S. Pat. No. 6,241,777. Other examples of phakic intraocular lenses are described in, e.g., U.S. Pat. Nos. 6,228,115; 5,480,428 and 5,222,981.

In another aspect, the intraocular lens may be a multi-focal lens capable of variable accommodation to enable the user to look through different portions of the lens to achieve different levels of focusing power. For example, the intraocular lens may be a variable focus lens composed of two lens portions with an optical zone between the lenses which may contain a fluid reservoir and channel containing charged solution. See, e.g., U.S. Pat. No. 5,443,506.

In another aspect, intraocular lenses may be deformable such that the lens may be folded for insertion through a tunnel incision. For example, the intraocular lens may be composed of a lens with fixation members for retaining the lens in the eye which may be configured for folding or rolling from a normal optical condition into an insertion condition to permit the lens to be passed through an incision into the eye. See, e.g., U.S. Pat. No. 5,476,513. The intraocular lens may be composed of a resilient, deformable silicone based optic with a fixation means coupled to the optic for retaining the optic in the eye. See, e.g., U.S. Pat. No. 5,201,763. The intraocular lens may be composed of a copolymer of three constituents which may be deformable from its original shape. See, e.g., U.S. Pat. No. 5,359,021. The intraocular lens may be composed of a transparent, flexible membrane with an interior sac and an attached bladder, in which optical fluid medium is shunted from the optical element to the bladder to aid in its deformity during insertion. See, e.g., U.S. Pat. No. 6,048,364. The intraocular lens may be a biocomposite composed of an optic portion made of high water content hydrogel capable of being folded and a haptic portion of low water content hydrogel having strength and rigidity. See, e.g., U.S. Pat. No. 5,211,662. Other deformable intraocular lenses are described in, e.g., U.S. Pat. Nos. 6,267,784; 5,507,806 and U.S. patent application Publication No. 2003/0114928A1.

Other related devices and/or compositions (e.g., insertion devices) that may be used in conjunction with intraocular lenses are described in, e.g., U.S. Pat. Nos. 6,629,979; 6,187,042; 6,113,633; 4,740,282 and U.S. patent application Publication Nos. 2003/0212409A1 and 2003/0187455A1

Intraocular lenses, which may benefit from having the subject polymer composition infiltrated into adjacent tissue according to the present invention, include commercially available products. For example, Alcon Laboratories, Inc. (Fort Worth, Tex.) sells the foldable ACRYSOF Intraocular Lens. Bausch & Lomb Surgical, Inc. (San Dimas, Calif.) sells the foldable SOFLEX SE Intraocular Lens. Advanced Medical Optics, Inc (Santa Ana, Calif.) sells the CLARIFLEX Foldable Intraocular Lens, SENSAR Acrylic Intraocular Lens, and PHACOFLEX II SI40NB and SI30NB.

The intraocular implants of the invention may be used in various surgical procedures. For example, the intraocular implant may be used in conjunction with a transplant for the cornea. Synthetic corneas can be used in patients losing vision due to a degenerative cornea. Implanted synthetic corneas can restore patient vision, however, they often induce a fibrous foreign body response that limits their use. The intraocular implant of the present invention can prevent the foreign body response to the synthetic cornea and extend the cornea longevity. In another example, the synthetic cornea itself is coated with the polymer compositions of the invention, thus minimizing tissue reaction to corneal implantation.

In another aspect, the intraocular lens may be used in conjunction with treatment of secondary cataract after extracapsular cataract extraction.

As described above, the present invention provides intraocular lenses and other implants having the subject polymer compositions infiltrated into adjacent tissue, where the subject polymer compositions may include a therapeutic agent (e.g., an anti-scarring and/or anti-infective agent). In one aspect, the anti-scarring agent is not paclitaxel or a derivative thereof.

Numerous polymeric and non-polymeric delivery systems for use in intraocular implants have been described above.

Polymeric compositions may be infiltrated around implanted intraocular implants by applying the composition directly and/or indirectly into and/or onto (a) tissue adjacent to the intraocular implant; (b) the vicinity of the intraocular implant-tissue interface; (c) the region around the intraocular implant; and (d) tissue surrounding the intraocular implant. Methods for infiltrating the subject polymer compositions into tissue adjacent to an intraocular implant include delivering the polymer composition: (a) to the intraocular implant surface (e.g., as an injectable, paste, gel or mesh) during the implantation procedure; (b) to the surface of the tissue (e.g., as an injectable, paste, gel, in situ forming gel or mesh) immediately prior to, or during, implantation of the intraocular implant; (c) to the surface of the intraocular implant and/or the tissue surrounding the implanted intraocular implant (e.g., as an injectable, paste, gel, in situ forming gel or mesh) immediately after the implantation of the intraocular implant; (d) by topical application of the composition into the anatomical space where the intraocular implant may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the intraocular implant as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device.

The process of infiltrating the subject polymer compositions into tissue adjacent to these implants and the materials selected for these processes are such that they do not significantly alter the refractive index of the intraocular implant or the visible light transmission of the implant or lens.

According to one aspect, any fibrosis-inhibiting and/or anti-infective agent described above may be utilized in the practice of the present invention. In one aspect of the invention, the subject polymer compositions infiltrated into tissue adjacent to intraocular implants may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As intraocular implants are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

Hypertrophic Scars and Keloids

In another aspect, the subject polymer compositions may be infiltrated into tissue adjacent to a device for use in treating hypertrophic scars and keloids. The subject polymer compositions may contain a therapeutic agent (e.g., an anti-scarring and/or anti-infective agent).

A variety of devices for treating hypertrophic scars and keloids have been described. For example, the device may be an external tissue expansion device composed of two suture steel plates with adhesive attached foam cushions which apply constant continuous low grade force to skin and tissue to provide removal of hypertrophic scars and keloids. See, e.g., U.S. Pat. No. 6,254,624. The device may be a masking element which is pressed onto the scar tissue with an adjustable force by means of a pressure control unit and is connected with inflatable or suction members in the masking element. See, e.g., U.S. Pat. No. 6,013,094. The treatment may be a device having locking elements and grasping structures such that the dermal and epidermal layers of a skin wound can be pushed together such that the tissue edges are abutting, such that a wound may be closed with minimal scarring. See, e.g., U.S. Pat. No. 5,591,206.

In another aspect, the hypertrophic scar or keloid may be treated by using a device in conjunction with a coating or sheet that may be used to deliver either anti-scarring and/or anti-infective agents alone, or anti-scarring and/or anti-infective compositions as described above. For example, the coating or sheet may be a copolymer composed of a hydrophilic polymer, such as polyethylene glycol, that is bound to a polymer that adsorbs readily to the surfaces of body tissues, such as phenylboronic acid. See, e.g., U.S. Pat. No. 6,596,267. The coating or sheet may be a self-adhering silicone sheet which is impregnated with an antioxidant and/or antimicrobial. See, e.g., U.S. Pat. No. 6,572,878. The coating or sheet may be a wound dressing garment composed of an outer pliable layer and a self-adhesive inner gel lining which serves as a dressing for contacting wounds. See, e.g., U.S. Pat. No. 6,548,728. The coating or sheet may be a liquid composition composed of a film-forming carrier such as a collodion which contains one or more active ingredients such as a topical steroid, silicone gel and vitamin E. See, e.g., U.S. Pat. No. 6,337,076. The coating or sheet may be a bandage with a scar treatment pad with a layer of silicone elastomer or silicone gel. See, e.g., U.S. Pat. Nos. 6,284,941 and 5,891,076.

Treatments and devices used for hypertrophic scars and keloids, which may be combined with infiltration of the subject polymer compositions into adjacent tissue, or into hypertrophic scar and keloid tissue, according to the present invention, include commercially available products. Representative products include, for example, PROXIDERM External Tissue Expansion product for wound healing from Progressive Surgical Products (Westbury, N.Y.), CICA-CARE Gel Sheet dressing product from Smith & Nephew Healthcare Ltd. (India), and MEPIFORM Self-Adherent Silicone Dressing from Molnlycke Health Care (Eddystone, Pa.).

In one aspect, devices for the treatment of hypertrophic scars and keloids may have the subject polymer compositions infiltrated into adjacent tissue, where the subject polymer compositions may include a therapeutic agent (e.g., an anti-scarring and/or anti-infective agent). The polymer compositions may be a topical or injectable polymer composition that includes an anti-scarring and/or anti-infective agent and a polymeric carrier suitable for application on or into hypertrophic scars or keloids. Incorporation of a fibrosis-inhibiting and/or anti-infective agent into a topical formulation or an injectable formulation is one approach to treat this condition. The topical formulation can be in the form of a solution, a suspension, an emulsion, a gel, an ointment, a cream, film or mesh. The injectable formulation can be in the form of a solution, a suspension, an emulsion or a gel. Polymeric and non-polymeric components that can be used to prepare these topical or injectable compositions are described above.

Polymeric compositions may be infiltrated around devices used for hypertrophic scars and keloids by applying the composition directly and/or indirectly into and/or onto (a) tissue adjacent to the device used for hypertrophic scars and keloids; (b) the vicinity of the tissue interface with the device used for hypertrophic scars and keloids; (c) the region around the device used for hypertrophic scars and keloids; and (d) tissue surrounding the device used for hypertrophic scars and keloids. Methods for infiltrating the subject polymer compositions into tissue adjacent to a device used for hypertrophic scars and keloids include delivering the polymer composition: (a) to the surface of the device used for hypertrophic scars and keloids (e.g., as an injectable, paste, gel or mesh) during the implantation procedure; (b) to the surface of the tissue (e.g., as an injectable, paste, gel, in situ forming gel or mesh) immediately prior to, or during, implantation of the device used for hypertrophic scars and keloids; (c) to the surface of the device used for hypertrophic scars and keloids and/or the tissue surrounding the implanted device used for hypertrophic scars and keloids (e.g., as an injectable, paste, gel, in situ forming gel or mesh) immediately after the implantation of the device used for hypertrophic scars and keloids; (d) by topical application of the composition into the anatomical space where the device used for hypertrophic scars and keloids may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the device used for hypertrophic scars and keloids as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device.

According to one aspect, any fibrosis-inhibiting and/or anti-infective agent described above may be utilized in the practice of the present invention. In one aspect of the invention, the subject polymer compositions infiltrated into tissue adjacent to devices for the treatment of hypertrophic scars and keloids may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As devices for the treatment of hypertrophic scars and keloids are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation; the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g, 5-fluorouracil), folic acid antagonists (e.g., methotrexate and/or podophylotoxins (e.g., etoposide) may be utilized to enhance the antibacterial activity of the composition.

Vascular Grafts

In one aspect, the present invention provides for infiltration of the subject polymer compositions into tissue adjacent to a vascular graft. Vascular graft devices having a polymer composition containing a fibrosis-inhibiting and/or anti-infective agent infiltrated into adjacent tissue are capable of inhibiting or reducing the overgrowth of granulation tissue and/or inhibiting or preventing infection, which can improve the clinical efficacy of these devices. The subject polymer compositions may contain a therapeutic agent (e.g., an anti-scarring and/or anti-infective agent).

The vascular graft may be an extravascular graft or an intravascular (i.e., endoluminal) graft. The vascular graft may be, without limitation, in the form of a peripheral bypass application or a coronary bypass application. Vascular grafts may be used to replace or substitute damaged or diseased veins and arteries, including, without limitation, blood vessels damaged by aneurysms, intimal hyperplasia and thrombosis. Vascular grafts may also be used to provide access to blood vessels, for example, for hemodialysis access. Vascular grafts are implanted, for example, to provide an alternative conduit for blood flow through damaged or diseased areas in veins and arteries, including, without limitation, blood vessels damaged by aneurysms, intimal hyperplasia and thrombosis, however, the graft may lead to further complications, including, without limitation, infections, inflammation, thrombosis and intimal hyperplasia. The lack of long-term patency with vascular grafts may be due, for example, to surgical injury and abnormal hemodynamics and material mismatch at the suture line. Typically, further disease (e.g., restenosis) of the vessel occurs along the bed of the artery.

Some forms of improvements to vascular grafts have been made in an attempt to reduce the restenosis that occurs at the anastomosis site. Improvements include: (a) using a Miller cuff, which is a small piece of natural vein to make a short cuff that is joined by stitching it to the artery opening and the prosthetic graft; (b) using a flanged graft whereby the graft has a terminal skirt or cuff that facilitates an end-to-side anastomosis; (c) using a graft with an enlarged chamber having a large diameter for suture at the anastomosis site; and (d) using a graft that dispensing an agent that prevents thrombosis and/or intimal hyperplasia.

Representative examples of vascular grafts include, without limitation, synthetic bypass grafts (e.g., femoral-popliteal, femoral-femoral, axillary-femoral, and the like), vein grafts (e.g., peripheral and coronary), and internal mammary (e.g., coronary) grafts, bifurcated vascular grafts, intraluminal grafts, endovascular grafts and prosthetic grafts. Synthetic grafts can be made from a variety of polymeric materials, such as, for example, polytetrafluoroethylene (e.g., ePTFE), polyesters such as DACRON, polyurethanes, and combinations of polymeric materials.

Endoluminal vascular grafts may be used to treat aneurysms. For example, the vascular graft may be composed of a tubular graft with two tubular self-expanding stents that may be implanted for the treatment of aneurysms by means of minimally invasive procedures. See, e.g., U.S. Pat. No. 6,168,620. The vascular graft may be composed of a flexible tubular body and a compressible frame positioned against the tubular body for support which has pores on the surface to promote ingrowth. See, e.g., U.S. Pat. No. 5,693,088. The vascular graft may be bifurcated endovascular graft having a tubular trunk and two tubular limbs. See, e.g., U.S. Pat. No. 6,454,796. The vascular graft may be a kink-resistant endoluminal bifurcated graft having two separate lumens contacted by a single lumen section. See, e.g., U.S. Pat. No. 6,551,350. The vascular graft may be an intraluminal tube composed of ePTFE that has a seamline formed by overlapping the edges such that the microstructure fibrils are oriented in perpendicular directions. See, e.g., U.S. Pat. No. 5,718,973.

In another aspect, the vascular graft may be used as a conduit to bypass vascular stenosis or other vascular abnormalities. For example, the vascular graft may be composed of a porous material having a layer of porous hollow fibers positioned along the inner surface which allows for tissue growth while inhibiting bleeding during the healing process. See, e.g., U.S. Pat. No. 5,024,671. The vascular graft may be a flexible, monolithic, reinforced polymer tube having a microporous ePTFE tubular member and external ePTFE rib members projecting outwardly from the outer wall. See, e.g., U.S. Pat. No. 5,609,624. The vascular graft may be composed of a tubular wall having longitudinally extending pleats that respond flexurally to changes in blood pressure while maintaining high compliance with reduced kinking. See, e.g., U.S. Pat. No. 5,653,745. The vascular graft may be a radially supported ePTFE tube that is reinforced with greater density ring-shaped regions. See, e.g., U.S. Pat. No. 5,747,128. The vascular graft may be porous PTFE tubing composed of a microstructure of nodes interconnected by fibrils which has a coating of elastomer on the outer wall. See, e.g., U.S. Pat. Nos. 5,152,782 and 4,955,899. The vascular graft may be a plurality of polymeric fibers knitted together composed of at least three different fibers in which two fibers are absorbable and one is non-absorbable. See, e.g., U.S. Pat. Nos. 4,997,440; 4,871,365 and 4,652,264.

In another aspect, the vascular graft may be modified to reduce thrombus formation or intimal hyperplasia at the anastomotic site. For example, the vascular graft may have an enlarged chamber having a first diameter parallel to the axis of the tubular wall and a second diameter transverse to the axis of the tube. See, e.g., U.S. Pat. No. 6,589,278. The vascular graft may have a flanged skirt or cuff section with facilitates an end-to-side anastomosis directly between the artery and the end of the flanged bypass graft. See, e.g., U.S. Pat. No. 6,273,912. The vascular graft may be composed of a tubular wall having a non-thrombogenic agent within the luminal layer and a thrombogenic layer forming the exterior of the vascular graft. See, e.g., U.S. Pat. No. 6,440,166. The vascular graft may be composed of a smooth luminal surface made of ePTFE with a small pore size to reduce adherence of occlusive blood components. See, e.g., U.S. Pat. No. 6,517,571. The vascular graft may be composed of hollow tubing that contains drug that is helically wrapped around the outer wall of a porous ePTFE graft whereby drug is dispensed by infusion through the porous interstices of the graft wall. See, e.g., U.S. Pat. No. 6,355,063.

In another aspect, the vascular graft may be a harvested blood vessel that is used for bypass grafting. For example, vascular grafts may be composed of harvested arterial vessels from a host, such as the internal mammary arteries or inferior epigastric arteries. See, e.g., U.S. Pat. No. 5,797,946. Vascular grafts may also be composed of saphenous veins which may be harvested from the host and used for coronary bypass or peripheral bypass procedures. See, e.g., U.S. Pat. No. 6,558,313.

Other examples of vascular grafts are described in U.S. Pat. Nos. 3,096,560, 3,805,301, 3,945,052, 4,140,126, 4,323,525, 4,355,426, 4,475,972, 4,530,113, 4,550,447, 4,562,596, 4,601,718, 4,647,416, 4,878,908, 5,024,671, 5,104,399, 5,116,360, 5,151,105, 5,197,977, 5,282,824, 5,405,379, 5,609,624, 5,693,088, and 5,910,168.

Vascular grafts, which may from having the subject polymer composition infiltrated into adjacent tissue according to the present invention, include commercially available products. GORE-TEX Vascular Grafts and GORE-TEX INTERING Vascular Grafts are sold by Gore Medical Division (W. L. Gore & Associates, Inc. Newark, Del.). C.R. Bard, Inc. (Murray Hill, N.J.) sells the DISTAFLO Bypass Grafts and IMPRA CARBOFLO Vascular Grafts.

In one aspect, the present invention provides vascular grafts having the subject polymer compositions infiltrated into adjacent tissue, where the subject polymer compositions may include a therapeutic agent (e.g., an anti-scarring and/or anti-infective agent)

Numerous polymeric and non-polymeric delivery systems for use in connection with vascular grafts have been described above.

Polymeric compositions may be infiltrated around implanted vascular grafts by applying the composition directly and/or indirectly into and/or onto (a) tissue adjacent to the vascular graft; (b) the vicinity of the vascular graft-tissue interface; (c) the region around the vascular graft; and (d) tissue surrounding the vascular graft. Methods for infiltrating the subject polymer compositions into tissue adjacent to a vascular graft include delivering the polymer composition: (a) to the vascular graft surface (e.g., as an injectable, paste, gel or mesh) during the implantation procedure; (b) to the surface of the tissue (e.g., as an injectable, paste, gel, in situ forming gel or mesh) immediately prior to, or during, implantation of the vascular graft; (c) to the surface of the vascular graft and/or the tissue surrounding the implanted vascular graft (e.g., as an injectable, paste, gel, in situ forming gel or mesh) immediately after the implantation of the vascular graft; (d) by topical application of the composition into the anatomical space where the vascular graft may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the vascular graft as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device.

In addition to the fibrosis-inhibiting and/or anti-infective agent, the subject polymer compositions infiltrated into tissue adjacent to vascular graft devices can also further contain an anti-inflammatory agent (e.g., dexamethazone or aspirin) and/or an anti-thrombotic agent (e.g., heparin, heparin complexes, hydrophobic heparin derivatives, dipyridamole, or aspirin). The combination of agents may be contained in the polymer composition infiltrated into tissue adjacent to the vascular graft such that the thrombogenicity and/or fibrosis is reduced or inhibited. In certain embodiments, these agents may be contained in biodegradable polymers. For example, polymeric material that forms a gel in the pores and/or on the surface of the graft may be used, such as alginates, chitosan and chitosan sulfate, hyaluronic acid, dextran sulfate, PLURONIC polymers, chain extended PLURONIC polymers, polyester-polyether block copolymers of the various configurations (e.g., MePEG-PLA, PLA-PEG-PLA, and the like).

According to one aspect, any anti-scarring and/or anti-infective agent described above may be utilized in the practice of the present invention. In one aspect of the invention, the subject polymer compositions infiltrated into tissue adjacent to vascular grafts may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As vascular grafts are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

Hemodialysis Access Devices

In one aspect, the subject polymer compositions may be infiltrated into tissue adjacent to a hemodialysis access device. The subject polymer compositions may contain a therapeutic agent (e.g., an anti-scarring and/or anti-infective agent). Hemodialysis dialysis access devices that include a fibrosis-inhibiting and/or anti-infective agent are capable of inhibiting or reducing the overgrowth of granulation tissue and/or inhibiting or preventing infection, which can improve the clinical efficacy of these devices.

Hemodialysis access devices may be used when blood needs to be removed, cleansed and then returned to the body. Hemodialysis regulates the body's fluid and chemical balances as well as removes waste from the blood stream that cannot be cleansed by a normally functioning kidney due to disease or injury. For hemodialysis to occur, the blood may be obtained through a hemodialysis access or vascular access, in which minor surgery is performed to provide access through an AV fistula or AV access graft. These hemodialysis access devices may develop complications, including infections, inflammation, thrombosis and intimal hyperplasia of the associated blood vessels. The lack of long-term patency with hemodialysis access may be due to surgical injury, abnormal hemodynamics and material mismatch at the suture line. Typically, further disease (e.g., restenosis) of the vessel occurs along the bed of the artery and/or at the site of anastomosis.

In addition to the AV fistulas and AV access grafts described above, implantable subcutaneous hemodialysis access systems such as the commercially available catheters, ports, and shunts, may also be used for hemodialysis patients. These access systems may consist of a small metallic or polymeric device or devices implanted underneath the skin. These devices may be connected to flexible tubes, which are inserted into a vessel to allow for blood access.

Representative examples of hemodialysis access devices include, without limitation, AV access grafts, venous catheters, vascular grafts, implantable ports, and AV shunts. Synthetic hemodialysis access devices can be made from metals or polymers, such as polytetrafluoroethylene (e.g., ePTFE), polyesters such as DACRON, polyurethanes, or combinations of these materials.

In one aspect, the hemodialysis access device may be an AV access graft. For example, the AV access graft may be composed of an implantable self-expanding flexible percutaneous stent-graft of open weave construction with ends being compressible and having an elastic layer arranged along a portion of its length. See, e.g., U.S. Pat. Nos. 5,755,775 and 5,591,226. The AV access graft may be composed of a tubular-section with a generally constant diameter which tapers towards the venous end. See, e.g., U.S. Pat. No. 6,585,762. The AV access graft may be composed of a two microporous ePTFE tubes that are circumferentially disposed over each other with a polymeric layer interposed between such that the graft is self-sealing and exhibits superior radial tensile strength and suture hole elongation resistance. See, e.g., U.S. Pat. No. 6,428,571. The AV access graft may be composed of a coaxial double lumen tube with an inner and outer tube having a self-sealing, nonbiodegradable, polymeric adhesive between the tubes. See, e.g., U.S. Pat. No. 4,619,641. The AV access graft may be composed of a synthetic fabric having a high external velour profile which is woven or knitted to form a tubular prosthesis which has elastic fibers that allows self-sealing following a punctured state. See, e.g., U.S. Pat. No. 6,547,820. The AV access graft may be of tubular form having a base tube with the ablumenal surface covered with a deflectable material, such as a porous film, which is arranged adjacently to allow movement. See, e.g., U.S. Pat. No. 5,910,168.

In another aspect, the hemodialysis access device may be a catheter system. For example, the catheter system may be composed of a suction and return line that are adapted for disposition in the vascular system of the body and are connected to a subcutaneous connector port. See, e.g., U.S. Pat. Nos. 6,620,118 and 5,989,206. The catheter system may be an apparatus that is used to arterialize a vein by creating an AV fistula by inserting a catheter into a vein and a catheter into an adjacent artery. See, e.g., U.S. Pat. No. 6,464,665. The catheter system may be composed of a hollow sheath that provides percutaneous introduction of fistula-generating vascular catheters through a perforation in a vessel wall, such that the catheters generate an intervascular fistula on-demand between adjacent vessels. See, e.g., U.S. Pat. Nos. 6,099,542 and 5,830,224.

In another aspect, the hemodialysis access device may be used for an AV fistula. For example, the hemodialysis access device may be an AV fistula assembly composed of a synthetic coiled stent graft with helically-extending turns with gaps used to enhance the function of an AV fistula. See, e.g., U.S. Pat. No. 6,585,760.

In another aspect, the hemodialysis access device may be an implantable access port, shunt or valve. These devices may be implanted subcutaneously with communication to the blood supply and accessed using a percutaneous puncture. For example, the hemodialysis access device may be composed of housing having an entry port and an exit port to a passageway which has an elastomeric sealing valve that provides access into the exit port for a needle. See, e.g., U.S. Pat. No. 5,741,228. The hemodialysis access device may be a shunt composed of a slideable valve and flexible lid that has a fluid communication tube between the arterial and venous ends. See, e.g., U.S. Pat. No. 5,879,320. The hemodialysis access device may be a shunt in the form of a junction that has a connector with two legs that are inserted into the native blood vessel and one leg that is adapted for sealing to another blood vessel without punctures. See, e.g., U.S. Pat. No. 6,019,788. The hemodialysis access device may be a surface access double hemostatic valve that may be mounted on the wall of an AV graft for hemodialysis access. See, e.g., U.S. Pat. Nos. 6,004,301 and 6,090,067.

Hemodialysis access devices, which may benefit from having the subject polymer composition infiltrated into adjacent tissue according to the present invention, include commercially available products. For example, hemodialysis access devices include products, such as the LIFESITE (Vasca Inc., Tewksbury, Mass.) and the DIALOCK catheters from Biolink Corp. (Middleboro, Mass.), VECTRA Vascular Access Grafts and VENAFLO Vascular Grafts from C.R. Bard, Inc. (Murray Hill, N.J.), and GORE-TEX Vascular Grafts and Stretch Vascular Grafts from Gore Medical Division (W. L. Gore & Associates, Inc. Newark, Del.).

In one aspect, the present invention provides hemodialysis access devices having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with hemodialysis access devices have been described above.

Polymeric compositions may be infiltrated around implanted hemodialysis access devices by applying the composition directly and/or indirectly into and/or onto (a) tissue adjacent to the hemodialysis access device; (b) the vicinity of the hemodialysis access device -tissue interface; (c) the region around the hemodialysis access device; and (d) tissue surrounding the hemodialysis access device. Methods for infiltrating the subject polymer compositions into tissue adjacent to a hemodialysis access device include delivering the polymer composition: (a) to the hemodialysis access device surface (e.g., as an injectable, paste, gel or mesh) during the implantation procedure; (b) to the surface of the tissue (e.g., as an injectable, paste, gel, in situ forming gel or mesh) immediately prior to, or during, implantation of the hemodialysis access device; (c) to the surface of the hemodialysis access device and/or the tissue surrounding the implanted hemodialysis access device (e.g., as an injectable, paste, gel, in situ forming gel or mesh) immediately after the implantation of the hemodialysis access device; (d) by topical application of the composition into the anatomical space where the hemodialysis access device may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the hemodialysis access device as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device.

In addition to the fibrosis-inhibiting and/or anti-infective agent, subject polymer compositions infiltrated into tissue adjacent to hemodialysis access devices can also further contain an anti-inflammatory agent (e.g., dexamethazone or aspirin) and/or an anti-thrombotic agent (e.g., heparin, heparin complexes, hydrophobic heparin derivatives, dipyridamole, or aspirin).

According to the one aspect, any anti-scarring and/or anti-infective agent described above may be utilized in the practice of the present invention. In one aspect of the invention, the subject polymer compositions infiltrated into tissue adjacent to hemodialysis access devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As hemodialysis access devices are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm² -1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

Films and Meshes

In one aspect, the subject polymer compositions may be infiltrated into tissue adjacent to a film or mesh. The subject polymer compositions may contain a therapeutic agent (e.g., an anti-scarring and/or anti-infective agent). Infiltration of the subject polymer composition comprising a fibrosis-inhibiting agent and/or anti-infective agent into tissue adjacent to the film or mesh can minimize fibrosis (or scarring) in the vicinity of the implant and may reduce or prevent the formation of adhesions between the implant and the surrounding tissue and/or may inhibit or prevent infection in the vicinity of the implant site. In certain aspects, the film or mesh may be used as a drug-delivery vehicle (e.g., as a perivascular delivery device for the prevention of neointimal hyperplasia at an anastomotic site).

Films or meshes may take a variety of forms including, but not limited to, surgical barriers, surgical adhesion barriers, membranes (e.g., barrier membranes), surgical sheets, surgical patches (e.g., dural patches), surgical wraps (e.g., vascular, perivascular, adventitial, periadventitital wraps, and adventitial sheets), meshes (e.g., perivascular meshes), bandages, liquid bandages, surgical dressings, gauze, fabrics, tapes, surgical membranes, polymer matrices, shells, envelopes, tissue coverings, and other types of surgical matrices, scaffolds, and coatings.

In one aspect, the device comprises or may be in the form of a film. The film may be formed into one of many geometric shapes. Depending on the application, the film may be formed into the shape of a tube or may be a thin, elastic sheet of polymer. Generally, films are less than 5, 4, 3, 2, or 1 mm thick, more preferably less than 0.75 mm, 0.5 mm, 0.25 mm, or, 0.10 mm thick. Films can also be generated of thicknesses less than 50 μm, 25 μm or 10 μm. Films generally are flexible with a good tensile strength (e.g., greater than 50, preferably greater than 100, and more preferably greater than 150 or 200 N/cm²), good adhesive properties (i.e., adheres to moist or wet surfaces), and have controlled permeability. Polymeric films (which may be porous or non-porous) are particularly useful for application to the surface of a device or implant as well as to the surface of tissue, cavity or an organ.

Films may be made by several processes, including for example, by casting, and by spraying, or may be formed at the treatment site in situ. For example, a sprayable formulation may be applied onto the treatment site which then forms into a solid film.

In another aspect, the device may comprise or be in the form of a polymer, wherein at least some of the polymer is in the form of a mesh. A mesh, as used herein, is a material composed of a plurality of fibers or filaments (i.e., a fibrous material), where the fibers or filaments are arranged in such a manner (e.g., interwoven, knotted, braided, overlapping, looped, knitted, interlaced, intertwined, webbed, felted, and the like) so as to form a porous structure. Typically, a mesh is a pliable material, such that it has sufficient flexibility to be wrapped around the external surface of a body passageway or cavity, or a portion thereof. The mesh may be capable of providing support to the structure (e.g., the vessel or cavity wall) and may be adapted to release an amount of the therapeutic agent.

Mesh materials may take a variety of forms. For example, the mesh may be in a woven, knit, or non-woven form and may include fibers or filaments that are randomly oriented relative to each other or that are arranged in an ordered array or pattern. In one embodiment, for example, a mesh may be in the form of a fabric, such as, for example, a knitted, braided, crocheted, woven, non-woven (e.g., a melt-blown or wet-laid) or webbed fabric. In one embodiment, a mesh may include a natural or synthetic biodegradable polymer that may be formed into a knit mesh, a weave mesh, a sprayed mesh, a web mesh, a braided mesh, a looped mesh, and the like. Preferably, a mesh or wrap has intertwined threads that form a porous structure, which may be, for example, knitted, woven, or webbed.

The structure and properties of the mesh used in a device depend on the application and the desired mechanical (i.e., flexibility, tensile strength, and elasticity), degradation properties, and the desired loading and release characteristics for the selected therapeutic agent(s). The mesh should have mechanical properties, such that the device will remain sufficiently strong until the surrounding tissue has healed. Factors that affect the flexibility and mechanical strength of the mesh include, for example, the porosity, fabric thickness, fiber diameter, polymer composition (e.g., type of monomers and initiators), process conditions, and the additives that are used to prepare the material.

Typically, the mesh possesses sufficient porosity to permit the flow of fluids through the pores of the fiber network and to facilitate tissue ingrowth. Generally, the interstices of the mesh should be sufficiently wide apart to allow light visible by eye, or fluids, to pass through the pores. However, materials having a more compact structure also may be used. The flow of fluid through the interstices of the mesh depends on a variety of factors, including, for example, the stitch count or thread density. The porosity of the mesh may be further tailored by, for example, filling the interstices of the mesh with another material (e.g., particles or polymer) or by processing the mesh (e.g., by heating) in order to reduce the pore size and to create non-fibrous areas. Fluid flow through the mesh of the invention will vary depending on the properties of the fluid, such as viscosity, hydrophilicity/hydrophobicity, ionic concentration, temperature, elasticity, pseudoplasticity, particulate content, and the like. Preferably, the interstices do not prevent the release of impregnated or coated therapeutic agent(s) from the mesh, and the interstices preferably do not prevent the exchange of tissue fluid at the application site.

Mesh materials should be sufficiently flexible so as to be capable of being wrapped around all or a portion of the external surface of a body passageway or cavity. Flexible mesh materials are typically in the form of flexible woven or knitted sheets having a thickness ranging from about 25 microns to about 3000 microns; preferably from about 50 to about 1000 microns. Mesh material suitable for wrapping around arteries and veins typically ranges from about 100 to 400 microns in thickness.

The diameter and length of the fibers or filaments may range in size depending on the form of the material (e.g., knit, woven, or non-woven), and the desired elasticity, porosity, surface area, flexibility, and tensile strength. The fibers may be of any length, ranging from short filaments to long threads (i.e., several microns to hundreds of meters in length). Depending on the application, the fibers may have a monofilament or a multifilament construction.

The mesh may include fibers that are of same dimension or of different dimensions, and the fibers may be formed from the same or different types of biodegradable polymers. Woven materials, for example, may include a regular or irregular array of warp and weft strands and may include one type of polymer in the weft direction and another type (having the same or a different degradation profile from the first polymer) in the warp direction. The degradation profile of the weft polymer may be different than or the same as the degradation profile of the warp polymer. Similarly, knit materials may include one or more types (e.g., monofilament, multi-filament) and sizes of fibers and may include fibers made from the same or from different types of biodegradable polymers.

The structure of the mesh (e.g., fiber density and porosity) may impact the amount of therapeutic agent that may be loaded into or onto the device. For example, a fabric having a loose weave characterized by a low fiber density and high porosity will have a lower thread count, resulting in a reduced total fiber volume and surface area. As a result, the amount of agent that may be loaded into or onto, with a fixed carrier: therapeutic agent ratio, the fibers will be lower than for a fabric having a high fiber density and lower porosity. It is preferable that the mesh also should not invoke biologically detrimental inflammatory or toxic response, should be capable of being fully metabolized in the body, have an acceptable shelf life, and be easily sterilized.

The device may include multiple mesh materials in any combination or arrangement. For example, a portion of the device may be a knitted material and another portion may be a woven material. In another embodiment, the device may more than one layer (e.g., a layer of woven material fused to a layer of knitted material or to another layer of the same type or a different type of woven material). In some embodiments, multi-layer constructions (e.g., device having two or more layers of material) may be used, for example, to enhance the performance properties of the device (e.g., for enhancing the rigidity or for altering the porosity, elasticity, or tensile strength of the device) or for increasing the amount of drug loading.

Multi-layer constructions may be useful, for example, in devices containing more than one type of therapeutic agent. For example, a first layer of mesh material may be loaded with one type of agent and a second layer may be loaded with another type of agent. The two layers may be unconnected or connected (e.g., fused together, such as by heat welding or ultrasonic welding) and may be formed of the same type of fabric or from a different type of fabric having a different polymer composition and/or structure.

In certain aspects, a mesh may include portions that are not in the form of a mesh. For example, the device may include the form of a film, sheet, paste, and the like, and combinations thereof. For example, the device may have a multi-layer construction having a film layer that includes the therapeutic agent and one or more layers of mesh material. For example, the film layer may be interposed between two layers of mesh or may be disposed on just one side the mesh material. The film layer may include a first therapeutic agent, whereas one or more of the layers of mesh may include the same or a different agent. In another embodiment, the device includes at least two layers of mesh. In one aspect, at least two of the at least two layers of mesh are fused together.

In one aspect, multilayer devices are provided that may further include a film layer. The film layer may reside between two of the at least two layers of mesh. In yet another embodiment, a delivery device is described that includes a mesh, wherein the mesh includes a biodegradable polymer and a first therapeutic agent. The device may further include a film that includes a second therapeutic agent, which may have the same or a different composition than the first therapeutic agent. For example, in one embodiment, a device suitable for wrapping around a vein or artery includes a layer of mesh and a film layer loaded with a therapeutic agent. The device may be wrapped around a body passageway or cavity, such that the film layer contacts the external surface of the passageway or cavity. Thus, the device may deliver the appropriate dosage of agent and may provide sufficient mechanical strength to improve and maintain the structural integrity of the body passageway or cavity.

In one aspect, the mesh or film includes a polymer. The polymer may be a biodegradable polymer. Biodegradable compositions that may be used to prepare the mesh include polymers that comprise albumin, collagen, hyaluronic acid and derivatives, sodium alginate and derivatives, chitosan and derivatives gelatin, starch, cellulose polymers (for example methylcellulose, hydroxypropylcellulose, hydroxypropylmethylcellulose, carboxymethylcellulose, cellulose acetate phthalate, cellulose acetate succinate, hydroxypropylmethylcellulose phthalate), casein, dextran and derivatives, polysaccharides, poly(caprolactone), fibrinogen, poly(hydroxyl acids), poly(L-lactide) poly(D,L lactide), poly(D,L-lactide-co-glycolide), poly(L-lactide-co-glycolide), copolymers of lactic acid and glycolic acid, copolymers of ε-caprolactone and lactide, copolymers of glycolide and ε-caprolactone, copolymers of lactide and 1,4-dioxane-2-one, polymers and copolymers that include one or more of the residue units of the monomers D-lactide, L-lactide, D,L-lactide, glycolide, ε-caprolactone, trimethylene carbonate, 1,4-dioxane-2-one or 1,5-dioxepan-2-one, poly(glycolide), poly(hydroxybutyrate), poly(alkylcarbonate) and poly(orthoesters), polyesters, poly(hydroxyvaleric acid), polydioxanone, poly(ethylene terephthalate), poly(malic acid), poly(tartronic acid), polyanhydrides, polyphosphazenes, poly(amino acids). These compositions include copolymers of the above polymers as well as blends and combinations of the above polymers. (see generally, Illum, L., Davids, S. S. (eds.) “Polymers in Controlled Drug Delivery” Wright, Bristol, 1987; Arshady, J. Controlled Release 17:1-22, 1991; Pitt, Int. J. Phar. 59:173-196, 1990; Holland et al., J. Controlled Release 4:155-0180, 1986).

In one aspect, the mesh or film includes a biodegradable or resorbable polymer that is formed from one or more monomers selected from the group consisting of lactide, glycolide, e-caprolactone, trimethylene carbonate, 1,4-dioxan-2-one, 1,5-dioxepan-2-one, 1,4-dioxepan-2-one, hydroxyvalerate, and hydroxybutyrate. In one aspect, the polymer may include, for example, a copolymer of a lactide and a glycolide. In another aspect, the polymer includes a poly(caprolactone). In yet another aspect, the polymer includes a poly(lactic acid), poly(L-lactide)/poly(D,L-Lactide) blends or copolymers of L-lactide and D,L-lactide. In yet another aspect, the polymer includes a copolymer of lactide and e-caprolactone. In yet another aspect, the polymer includes a polyester (e.g., a poly(lactide-co-glycolide). The poly(lactide-co-glycolide) may have a lactide:glycolide ratio ranges from about 20:80 to about 2:98, a lactide:glycolide ratio of about 10:90, or a lactide:glycolide ratio of about 5:95. In one aspect, the poly(lactide-co-glycolide) is poly(L-lactide-co-glycolide). Other examples of biodegradable materials include polyglactin, polyglycolic acid, autogenous, heterogenous, and xenogeneic tissue (e.g., pericardium or small intestine submucosa), and oxidized, regenerated cellulose. These meshes can be knitted, woven or non-woven meshes. Examples of non-woven meshes include electrospun materials.

Meshes and films may be prepared from non-biodegradable polymers. Representative examples of non-biodegradable compositions include ethylene-co-vinyl acetate copolymers, acrylic-based and methacrylic-based polymers (e.g., poly(acrylic acid), poly(methylacrylic acid), poly(methylmethacrylate), poly(hydroxyethyl methacrylate), poly(alkylcynoacrylate), poly(alkyl acrylates), poly(alkyl methacrylates)), polyolefins such as poly(ethylene) or poly(propylene), polyamides (e.g., nylon 6,6), poly(urethanes) (e.g., poly(ester urethanes), poly(ether urethanes), poly(carbonate urethanes), poly(ester-urea)), polyesters (e.g., PET, polybutyleneterephthalate, and polyhexyleneterephthalate), olyethers (poly(ethylene oxide), poly(propylene oxide), poly(ethylene oxide)-poly(propylene oxide) copolymers, diblock and triblock copolymers, poly(tetramethylene glycol)), silicone containing polymers and vinyl-based polymers (polyvinylpyrrolidone, poly(vinyl alcohol), poly(vinyl acetate phthalate), poly(styrene-co-isobutylene-co-styrene), fluorine containing polymers (fluoropolymers) such as fluorinated ethylene propylene (FEP) or polytetrafluoroethylene (e.g., expanded PTFE).

The mesh or film material may comprise a combination of the above-mentioned biodegradable and non-degradable polymers. Further examples of polymers that may be used are either anionic (e.g., alginate, carrageenin, hyaluronic acid, dextran sulfate, chondroitin sulfate, carboxymethyl dextran, caboxymethyl cellulose and poly(acrylic acid)), or cationic (e.g., chitosan, poly-I-lysine, polyethylenimine, and poly(allyl amine)) (see generally, Dunn et al., J. Applied Polymer Sci. 50:353, 1993; Cascone et al., J. Materials Sci.: Materials in Medicine 5:770, 1994; Shiraishi et al., Biol. Pharm. Bull. 16:1164, 1993; Thacharodi and Rao, Int'l J. Pharm. 120:115, 1995; Miyazaki et al., Int'l J. Pharm. 118:257, 1995). Preferred polymers (including copolymers and blends of these polymers) include poly(ethylene-co-vinyl acetate), poly(carbonate urethanes), poly(hydroxyl acids) (e.g., poly(D,L-lactic acid) oligomers and polymers, poly(L-lactic acid) oligomers and polymers, poly(D-lactic acid) oligomers and polymers, poly(glycolic acid), copolymers of lactic acid and glycolic acid, copolymers of lactide and glycolide, poly(caprolactone), copolymers of lactide or glycolide and ε-caprolactone), poly(valerolactone), poly(anhydrides), copolymers prepared from caprolactone and/or lactide and/or glycolide and/or polyethylene glycol.

A variety of polymeric and non-polymeric films and meshes have been described which may be combined with an anti-scarring agent. For example, the film or mesh may be a biodegradable polymeric matrix that conforms to the tissue and releases the agent in a controlled release manner. See, e.g., U.S. Pat. No. 6,461,640. The film or mesh may be a self-adhering silicone sheet which is impregnated with an antioxidant and/or antimicrobial. See, e.g., U.S. Pat. No. 6,572,878. The film or mesh may be a pliable shield with attachment ports and fenestrations that is adapted to cover a bony dissection in the spine. See, e.g., U.S. Pat. No. 5,868,745 and U.S. patent application No. 2003/0078588. The film or mesh may be a resorbable micro-membrane having a single layer of non-porous polymer base material of poly-lactide. See, e.g., U.S. Pat. No. 6,531,146 and U.S. application No. 2004/0137033. The film or mesh may be a flexible neuro decompression device that has an outer surface texturized with microstructures to reduce fibroplasia when it is wrapped around a nerve in a canal. See, e.g., U.S. Pat. No. 6,106,558. The film or mesh may be a resorbable collagen membrane that is wrapped around the spinal chord to inhibit cell adhesions. See, e.g., U.S. Pat. No. 6,221,109. The film or mesh may be a wound dressing garment composed of an outer pliable layer and a self-adhesive inner gel lining which serves as a dressing for contacting wounds. See, e.g., U.S. Pat. No. 6,548,728. The film or mesh may be a bandage with a scar treatment pad with a layer of silicone elastomer or silicone gel. See, e.g., U.S. Pat. Nos. 6,284,941 and 5,891,076. The film or mesh may be a crosslinkable system with at least three reactive compounds each having a polymeric molecular core with at least one functional group. See, e.g., U.S. Pat. No. 6,458,889. The film or mesh may be composed of a prosthetic fabric having a 3-dimensional structure separating two surfaces in which one is open to post-surgical cell colonization and one is linked to a film of collagenous material. See, e.g., U.S. Pat. No. 6,451,032. The film or mesh may be composed by crosslinking two synthetic polymers, one having nucleophilic groups and the other having electrophilic groups, such that they form a matrix that may be used to incorporate a biologically active compound. See, e.g., U.S. Pat. Nos. 6,323,278; 6,166,130; 6,051,648 and 5,874,500. The film or mesh may be a film composed of hetero-bifunctional anti-adhesion binding agents that act to covalently link substrate materials, such as collagen, to receptive tissue. See, e.g., U.S. Pat. No. 5,580,923. The film or mesh may be a conformable warp-knit fabric of oxidized regenerated cellulose or other bioresorbable material which acts like a physical barrier to prevent postoperative adhesions. See, e.g., U.S. Pat. No. 5,007,916. Meshes for use in the practice of the invention also are described in U.S. Pat. No. 6,575,887, and co-pending application, entitled “Perivascular Wraps,” filed Sep. 26, 2003 (U.S. Ser. No. (U.S. Ser. No. 10/673,046).

In one aspect, the mesh may be suitable for use in hernia repair surgery or in other types of surgical procedures. Mesh fabrics for use in connection with hernia repairs are disclosed in U.S. Pat. Nos. 6,638,284; 5,292,328; 4,769,038 and 2,671,444. Surgical meshes may be produced by knitting, weaving, braiding, or otherwise forming a plurality of yarns (e.g., monofilament or multifilament yarns made of polymeric materials such as polypropylene and polyester) into a support trellis. Knitted and woven fabrics constructed from a variety of synthetic fibers and the use of the fabrics, in surgical repair are also discussed in U.S. Pat. Nos. 3,054,406; 3,124,136; 4,193,137; 4,347,847; 4,452,245; 4,520,821; 4,633,873; 4,652,264; 4,655,221; 4,838,884 and 5,002,551 and European Patent Application No. 334,046. Implantable hernia meshes are described in U.S. Pat. Nos. 6,610,006; 6,368,541 and 6,319,264. Hernia meshes for the repair of hiatal hernias are described in, e.g., U.S. Pat. No. 6,436,030. Hernia meshes for the repair of abdominal (e.g., ventral and umbilical) hernias are described in U.S. Pat. No. 6,383,201. Infection-resistant hernia meshes are described in, e.g., U.S. Pat. No. 6,375,662. Hernia meshes such as those described in the patents listed above are suitable for combining with a fibrosis-inducing agent to create a mesh which promotes the growth of fibrous tissue.

In one aspect, the fibrosis-inhibiting agent can be incorporated into a biodegradable or dissolvable film or mesh that is then applied to the treatment site prior or post implantation of the prosthesis/implant. Exemplary materials for the manufacture of these films or meshes are hyaluronic acid (crosslinked or non-crosslinked), cellulose derivatives (e.g., hydroxypropyl cellulose), PLGA, collagen and crosslinked poly(ethylene glycol).

The film or mesh may be in the form of a tissue graft, which may be an autograft, allograft, biograft, biogenic graft or xenograft. Tissue grafts may be derived from various tissue types. Representative examples of tissues that may be used to prepare biografts include, but are not limited to, rectus sheaths, peritoneum, bladder, pericardium, veins, arteries, diaphragm and pleura. The biograft may be harvested from a host, loaded with an anti-scarring agent and then applied in a perivascular manner at the site where lesions and intimal hyperplasia can develop (e.g., at an anastomotic site). Once implanted, the agent (e.g., paclitaxel) is released from the graft and can penetrate the vessel wall to prevent the formation of intimal hyperplasia at the treatment site. In certain embodiments, the biograft may be used as a backing layer to enclose a composition (e.g., a gel or paste loaded with anti-scarring agent).

Films and meshes, which may benefit from having the subject polymer composition infiltrated into adjacent tissue according to the present invention, include commercially available products. Examples of films and meshes into which a fibrosis agent can be incorporated include INTERCEED (Johnson & Johnson, Inc.), PRECLUDE (W. L. Gore), and POLYACTIVE (poly(ether ester) multiblock copolymers (Osteotech, Inc., Shrewsbury, N.J.), based on poly(ethylene glycol) and poly(butylene terephthalate), and SURGICAL absorbable hemostat gauze-like sheet from Johnson & Johnson. Another mesh is a prosthetic polypropylene mesh with a bioresorbable coating called SEPRAMESH Biosurgical Composite (Genzyme Corporation, Cambridge, Mass.). One side of the mesh is coated with a bioresorbable layer of sodium hyaluronate and carboxymethylcellulose, providing a temporary physical barrier that separates the underlying tissue and organ surfaces from the mesh. The other side of the mesh is uncoated, allowing for complete tissue ingrowth similar to bare polypropylene mesh. In one embodiment, the fibrosis-inducing agent may be applied only to the uncoated side of SEPRAMESH and not to the sodium hyaluronate/carboxymethylcellulose coated side. Other films and meshes include: (a) BARD MARLEX mesh (C.R. Bard, Inc.), which is a very dense knitted fabric structure with low porosity; (b) monofilament polypropylene mesh such as PROLENE available from Ethicon, Inc. Somerville, N.J. (see, e.g., U.S. Pat. Nos. 5,634,931 and 5,824,082)); (c) SURGISIS GOLD and SURGISIS IHM soft tissue graft (both from Cook Surgical, Inc.) which are devices specifically configured for use to reinforce soft tissue in repair of inguinal hernias in open and laparoscopic procedures; (d) thin walled polypropylene surgical meshes such as are available from Atrium Medical Corporation (Hudson, N.H.) under the trade names PROLITE, PROLITE ULTRA, and LITEMESH; (e) COMPOSIX hernia mesh (C.R. Bard, Murray Hill, N.J.), which incorporates a mesh patch (the patch includes two layers of an inert synthetic mesh, generally made of polypropylene, and is described in U.S. Pat. No. 6,280,453) that includes a filament to stiffen and maintain the device in a flat configuration; (f) VISILEX mesh (from C.R. Bard, Inc.), which is a polypropylene mesh that is constructed with monofilament polypropylene; (g) other meshes available from C.R. Bard, Inc. which include PERFIX Plug, KUGEL Hernia Patch, 3D MAX mesh, LHI mesh, DULEX mesh, and the VENTRALEX Hernia Patch; and (h) other types of polypropylene monofilament hernia mesh and plug products include HERTRA mesh 1, 2, and 2A, HERMESH 3, 4 & 5 and HERNIAMESH plugs T1, T2, and T3 from Herniamesh USA, Inc. (Great Neck, N.Y.).

Other examples of commercially available meshes which may benefit from having the subject polymer composition infiltrated into adjacent tissue are described below. One example includes a prosthetic polypropylene mesh with a bioresorbable coating sold under the trade name SEPRAMESH Biosurgical Composite (Genzyme Corporation). One side of the mesh is coated with a bioresorbable layer of sodium hyaluronate and carboxymethylcellulose, providing a temporary physical barrier that separates the underlying tissue and organ surfaces from the mesh. The other side of the mesh is uncoated, allowing for complete tissue ingrowth similar to bare polypropylene mesh. In one embodiment, the subject polymer composition comprising a fibrosis-inducing and/or anti-infective agent may be infiltrated into tissue adjacent only to the uncoated side of SEPRAMESH and not to the sodium hyaluronate/ carboxymethylcellulose coated side. Boston Scientific Corporation sells the TRELEX NATURAL Mesh which is composed of a unique knitted polypropylene material. Ethicon, Inc. makes the absorbable VICRYL (polyglactin 910) meshes (knitted and woven) and MERSILENE Polyester Fiber Mesh. Dow Corning Corporation (Midland, Mich.) sells a mesh material formed from silicone elastomer known as SILASTIC Rx Medical Grade Sheeting (Platinum Cured). United States Surgical/Syneture (Norwalk, Conn.) sells a mesh made from absorbable polyglycolic acid under the trade name DEXON Mesh Products. Membrana Accurel Systems (Obernburg, Germany) sells the CELGARD microporous polypropylene fiber and membrane. Gynecare Worldwide, a division of Ethicon, Inc. sells a mesh material made from oxidized, regenerated cellulose known as INTERCEED TC7. Integra LifeSciences Corporation (Plainsboro, N.J.) makes DURAGEN PLUS Adhesion Barrier Matrix, which can be used as a barrier against adhesions following spinal and cranial surgery and for restoration of the dura mater. HYDROSORB Shield from MacroPore Biosurgery, Inc. (San Diego, Calif.) is a film for temporary wound support to control the formation of adhesions in specific spinal applications.

Numerous polymeric and non-polymeric carrier systems that can be used in connection with films and meshes have been described above. Methods for incorporating the fibrosis-inhibiting compositions onto or into the film or mesh include: (a) affixing (directly or indirectly) to the film or mesh a fibrosis-inhibiting composition (e.g., by either a spraying process or dipping process as described above, with or without a carrier), (b) incorporating or impregnating into the film or mesh a fibrosis-inhibiting composition (e.g., by either a spraying process or dipping process as described above, with or without a carrier (c) by coating the film or mesh with a substance such as a hydrogel which will in turn absorb the fibrosis-inhibiting composition, (d) constructing the film or mesh itself or a portion of the film or mesh with a fibrosis-inhibiting composition, or (e) by covalently binding the fibrosis-inhibiting agent directly to the film or mesh surface or to a linker (small molecule or polymer) that is coated or attached to the film or mesh surface. For devices that are coated, the coating process can be performed in such a manner as to (a) coat only one surface of the film or mesh or (b) coat all or parts of both sides of the film or mesh.

In one aspect, the present invention provides a film or mesh may having the subject polymer compositions infiltrated into adjacent tissue, where the subject polymer compositions may include a therapeutic agent (e.g., an anti-scarring and/or anti-infective agent). In some embodiments, the polymer composition is a polymer composition that can function as a surgical adhesion barrier.

A variety of polymeric compositions have been described that may be used in conjunction with the films and meshes of the invention. Such compositions may be in the form of, for example, gels, sprays, liquids, and pastes, or may be polymerized from monomeric or prepolymeric constituents in situ. For example, the composition may be a polymeric tissue coating which is formed by applying a polymerization initiator to the tissue and then covering it with a water-soluble macromer that is polymerizable using free radical initiators under the influence of UV light. See, e.g., U.S. Pat. Nos. 6,177,095 and 6,083,524. The composition may be an aqueous composition including a surfactant, pentoxifylline and a polyoxyalkylene polyether. See, e.g., U.S. Pat. No. 6,399,624. The composition may be a hydrogel-forming, self-solvating, absorbable polyester copolymers capable of selective, segmental association into compliant hydrogels mass upon contact with an aqueous environment. See, e.g., U.S. Pat. No. 5,612,052. The composition may be composed of fluent pre-polymeric material that is emitted to the tissue surface and then exposed to activating energy in situ to initiate conversion of the applied material to non-fluent polymeric form. See, e.g., U.S. Pat. Nos. 6,004,547 and 5,612,050. The composition may be composed of a gas mixture of oxygen present in a volume ratio of 1 to 20%. See, e.g., U.S. Pat. No. 6,428,500. The composition may be composed of an anionic polymer having an acid sulfate and sulfur content greater than 5% which acts to inhibit monocyte or macrophage invasion. See, e.g., U.S. Pat. No. 6,417,173. The composition may be composed of a non-gelling polyoxyalkylene composition with or without a therapeutic agent. See, e.g., U.S. Pat. No. 6,436,425. The composition may be coated onto tissue surfaces and may be composed of an aqueous solution of a hydrophilic, polymeric material (e.g., polypeptides or polysaccharide) having greater than 50,000 molecular weight and a concentration range of 0.01% to 15% by weight. See, e.g., U.S. Pat. No. 6,464,970.

Other representative examples of polymeric compositions which may be infiltrated into tissue adjacent to the film or mesh include poly(ethylene glycol)-based systems, hyaluronic acid and crosslinked hyaluronic acid compositions. These compositions can be applied as the final composition or they can be applied as materials that form crosslinked gel in situ.

Other compositions that can be used in conjunction with films and meshes, include, but are not limited to: (a) sprayable PEG-containing formulations such as COSEAL, SPRAYGEL, FOCALSEAL or DURASEAL; (b) hyaluronic acid-containing formulations such as RESTYLANE, HYLAFORM, PERLANE, SYNVISC, SEPRAFILM, SEPRACOAT, INTERGEL, (c) polymeric gels such as REPEL or FLOWGEL, (d) dextran sulfate gels such as the ADCON range of products, (e) lipid based compositions such as ADSURF (Brittania Pharmaceuticals).

The film or mesh (or device comprising the film or mesh) may be made sterile either by preparing them under aseptic environment and/or they may be terminally sterilized using methods known in the art, such as gamma radiation or electron beam sterilization methods or a combination of both of these methods.

Films and meshes may be applied to any bodily conduit or any tissue that may be prone to the development of fibrosis or intimal hyperplasia. Prior to implantation, the film or mesh may be trimmed or cut from a sheet of bulk material to match the configuration of the widened foramen, canal, or dissection region, or at a minimum, to overlay the exposed tissue area. The film or mesh may be bent or shaped to match the particular configuration of the placement region. The film or mesh may also be rolled in a cuff shape or cylindrical shape and placed around the exterior periphery of the desired tissue. The film or mesh may be provided in a relatively large bulk sheet and then cut into shapes to mold the particular structure and surface topography of the tissue or device to be wrapped. Alternatively, the film or mesh may be pre-shaped into one or more patterns for subsequent use. The films and meshes may be typically rectangular in shape and be placed at the desired location within the surgical site by direct surgical placement or by endoscopic techniques. The film or mesh may be secured into place by wrapping it onto itself (i.e., self-adhesive), or by securing it with sutures, staples, sealant, and the like. Alternatively, the film or mesh may adhere readily to tissue and therefore, additional securing mechanisms may not be required.

The films or meshes of the invention may be used for a variety of indications, including, without limitation: (a) prevention of surgical adhesions between tissues following surgery (e.g., gynecologic surgery, vasovasostomy, hernia repair, nerve root decompression surgery and laminectomy); (b) prevention of hypertrophic scars or keloids (e.g., resulting from tissue burns or other wounds); (c) prevention of intimal hyperplasia and/or restenosis (e.g., resulting from insertion of vascular grafts or hemodialysis access devices); (d) may be used in affiliation with devices and implants that lead to scarring as described herein (e.g., as a sleeve or mesh around a breast implant to reduce or inhibit scarring); (e) prevention of infection (e.g., resulting from tissue burns, surgery or other wounds); or (f) may be used in affiliate with devices and implants that lead to infection as described herein.

In one embodiment, films or meshes may be used to prevent adhesions that occur between tissues following surgery, injury or disease. Adhesion formation, a complex process in which bodily tissues that are normally separate grow together, occurs most commonly as a result of surgical intervention and/or trauma. Generally, adhesion formation is an inflammatory reaction in which factors are released, increasing vascular permeability and resulting in fibrinogen influx and fibrin deposition. This deposition forms a matrix that bridges the abutting tissues. Fibroblasts accumulate, attach to the matrix, deposit collagen and induce angiogenesis. If this cascade of events can be prevented within 4 to 5 days following surgery, then adhesion formation can be inhibited. Adhesion formation or unwanted scar tissue accumulation and encapsulation complicates a variety of surgical procedures and virtually any open or endoscopic surgical procedure in the abdominal or pelvic cavity. Encapsulation of surgical implants also complicates breast reconstruction surgery, joint replacement surgery, hernia repair surgery, artificial vascular graft surgery, and neurosurgery. In each case, the implant becomes encapsulated by a fibrous connective tissue capsule which compromises or impairs the function of the surgical implant (e.g., breast implant, artificial joint, surgical mesh, vascular graft, dural patch). Chronic inflammation and scarring also occurs during surgery to correct chronic sinusitis or removal of other regions of chronic inflammation (e.g., foreign bodies, infections (fungal, mycobacterium). Surgical procedures that may lead to surgical adhesions may include cardiac, spinal, neurologic, pleural, thoracic and gynecologic surgeries. However, adhesions may also develop as a result of other processes, including, but not limited to, non-surgical mechanical injury, ischemia, hemorrhage, radiation treatment, infection-related inflammation, pelvic inflammatory disease and/or foreign body reaction. This abnormal scarring interferes with normal physiological functioning and, in come cases, can force and/or interfere with follow-up, corrective or other surgical operations. For example, these post-operative surgical adhesions occur in 60 to 90% of patients undergoing major gynecologic surgery and represent one of the most common causes of intestinal obstruction in the industrialized world. These adhesions are a major cause of failed surgical therapy and are the leading cause of bowel obstruction and infertility. Other adhesion-treated complications include chronic pelvic pain, urethral obstruction and voiding dysfunction.

Currently, preventative therapies, administered 4 to 5 days following surgery, are used to inhibit adhesion formation. Various modes of adhesion prevention have been examined, including (1) prevention of fibrin deposition, (2) reduction of local tissue inflammation, and (3) removal of fibrin deposits. Fibrin deposition is prevented through the use of physical adhesion barriers that are either mechanical or comprised of viscous solutions. Although many investigators are utilizing adhesion prevention barriers, a number of technical difficulties exist.

In one aspect, the present invention provides films and meshes having the subject polymer composition comprising an anti-scarring agent infiltrated into adjacent tissue for use as surgical adhesion barriers.

In one aspect, films and meshes having the subject polymer composition comprising an anti-scarring agent infiltrated into adjacent tissue may be used to prevent surgical adhesions in the epidural and dural tissue which is a factor contributing to failed back surgeries and complications associated with spinal injuries (e.g., compression and crush injuries). Scar formation within dura and around nerve roots has been implicated in rendering subsequent spine operations technically more difficult. To gain access to the spinal foramen during back surgeries, vertebral bone tissue is often disrupted. Back surgeries, such as laminectomies and diskectomies, often leave the spinal dura exposed and unprotected. As a result, scar tissue frequently forms between the dura and the surrounding tissue. This scar is formed from the damaged erector spinae muscles that overlay the laminectomy site. This results in adhesion development between the muscle tissue and the fragile dura, thereby, reducing mobility of the spine and nerve roots which leads to pain and slow post-operative recovery. To circumvent adhesion development, a scar-reducing barrier may be inserted between the dural sleeve and the paravertebral musculature post-laminotomy. This reduces cellular and vascular invasion into the epidural space from the overlying muscle and exposed cancellous bone and thus, reduces the complications associated with the canal housing the spinal chord and/or nerve roots.

In another aspect, films and meshes having the subject polymer composition comprising an anti-scarring agent infiltrated into adjacent tissue may be used to prevent the fibrosis from occurring between a hernia repair mesh and the surrounding tissue. Hernias are abnormal protrusions (outpouchings) of an organ or other body structure through a defect or natural opening in a covering membrane, muscle or bone. Hernias themselves are not dangerous, but can become extremely problematic if they become incarcerated. Surgical prostheses used in hernia repair (referred to herein as “hernia meshes”) include prosthetic mesh-or gauze-like materials, which support the repaired hernia or other body structures during the healing process. Hernias are often repaired surgically to prevent complications. Conditions in which a hernia mesh may need to be used include, without limitation, the repair of inguinal (i.e., groin), umbilical, ventral, femoral, abdominal, diaphragmatic, epigastric, gastroesophageal, hiatal, intermuscular, mesenteric, paraperitoneal, rectovaginal, rectocecal, uterine, and vesical hernias. Hernia repair typically involves returning the viscera to its normal location and the defect in the wall is primarily closed with sutures, but for bigger gaps, a mesh is placed over the defect to close the hernia opening. Infiltration of the subject polymer composition comprising an anti-scarring agent into tissue adjacent to a hernia repair mesh may reduce or prevent fibrosis proximate to the implanted hernia mesh, thereby minimizing the possibility of adhesions between the abdominal wall or other tissues and the mesh itself, and reducing further complications and abdominal pain.

In yet another aspect, films or meshes having the subject polymer composition comprising an anti-scarring agent infiltrated into adjacent tissue may be used to prevent hypertrophic scars or keloids (e.g., resulting from tissue burns or other wounds). Hypertrophic scars and keloids are the result of an excessive fibroproliferative wound healing response. Briefly, healing of wounds and scar formation occurs in three phases: inflammation, proliferation, and maturation. The first phase, inflammation, occurs in response to an injury which is severe enough to break the skin. During this phase, which lasts 3 to 4 days, blood and tissue fluid form an adhesive coagulum and fibrinous network which serves to bind the wound surfaces together. This is then followed by a proliferative phase in which there is ingrowth of capillaries and connective tissue from the wound edges, and closure of the skin defect. Finally, once capillary and fibroblastic proliferation has ceased, the maturation process begins wherein the scar contracts and becomes less cellular, less vascular, and appears flat and white. This final phase may take between 6 and 12 months. If too much connective tissue is produced and the wound remains persistently cellular, the scar may become red and raised. If the scar remains within the boundaries of the original wound it is referred to as a hypertrophic scar, but if it extends beyond the original scar and into the surrounding tissue, the lesion is referred to as a keloid. Hypertrophic scars and keloids are produced during the second and third phases of scar formation. Several wounds are particularly prone to excessive endothelial and fibroblastic proliferation, including burns, open wounds, and infected wounds. With hypertrophic scars, some degree of maturation occurs and gradual improvement occurs. In the case of keloids however, an actual tumor is produced which can become quite large. Spontaneous improvement in such cases rarely occurs. A film or mesh having the subject polymer composition comprising an anti-scarring agent infiltrated into adjacent tissue may be placed in contact with a wound or burn site in order to prevent formation of hypertrophic scar or keloids.

In yet another aspect, films and meshes having the subject polymer composition comprising an anti-scarring agent infiltrated into adjacent tissue are provided that may be used for delivering an anti-scarring agent to an external portion (surface) of a body passageway or cavity. Examples of body passageways include arteries, veins, the heart, the esophagus, the stomach, the duodenum, the small intestine, the large intestine, biliary tracts, the ureter, the bladder, the urethra, lacrimal ducts, the trachea, bronchi, bronchiole, nasal airways, Eustachian tubes, the external auditory mayal, vas deferens and fallopian tubes. Examples of cavities include the abdominal cavity, the buccal cavity, the peritoneal cavity, the pericardial cavity, the pelvic cavity, perivisceral cavity, pleural cavity and uterine cavity.

Examples of conditions that may be treated or prevented with films and meshes having the subject polymer composition comprising an anti-scarring agent infiltrated into adjacent tissue include iatrogenic complications of arterial and venous catheterization, complications of vascular dissection, complications of gastrointestinal passageway rupture and dissection, restonotic complications associated with vascular surgery (e.g., bypass surgery), and intimal hyperplasia.

In one aspect, an anti-scarring agent may be delivered from the subject polymer composition infiltrated into tissue adjacent to a film or mesh to the external walls of body passageways or cavities for the purpose of preventing and/or reducing a proliferative biological response that may obstruct or hinder the optimal functioning of the passageway or cavity, including, for example, iatrogenic complications of arterial and venous catheterization, aortic dissection, cardiac rupture, aneurysm, cardiac valve dehiscence, graft placement (e.g., A-V-bypass, peripheral bypass, CABG), fistula formation, passageway rupture and surgical wound repair.

The films or meshes may be used in the form of a perivascular wrap to prevent restenosis at anastomotic sites resulting from insertion of vascular grafts or hemodialysis access devices. In this case, perivascular wraps having the subject polymer composition containing an anti-scarring agent infiltrated into adjacent tissue may be used in conjunction with a vascular graft to inhibit scarring at an anastomotic site. These films or meshes may be placed or wrapped in a perivascular (periadventitial) manner around the outside of the anastomosis at the time of surgery. Film and mesh implants having the subject polymer composition containing an anti-scarring agent infiltrated into adjacent tissue may be used with synthetic bypass grafts (femoral-popliteal, femoral-femoral, axillary-femoral etc.), vein grafts (peripheral and coronary), internal mammary (coronary) grafts or hemodialysis grafts (AV fistulas, AV access grafts).

In order to further the understanding of such conditions, representative complications leading to compromised body passageway or cavity integrity are discussed in more detail below.

In one aspect, the subject polymer compositions may be infiltrated into tissue adjacent to a coronary artery bypass graft (“CABG”). The subject polymer compositions may contain a therapeutic agent (e.g., an anti-scarring and/or anti-infective agent).

In one aspect, the subject polymer compositions may be infiltrated into tissue adjacent to a peripheral bypass surgery site. The subject polymer compositions may contain a therapeutic agent (e.g., an anti-scarring and/or anti-infective agent).

In one aspect, the subject polymer compositions may be infiltrated into tissue adjacent to an arterio-venous fistula. The subject polymer compositions may contain a therapeutic agent (e.g., an anti-scarring and/or anti-infective agent).

In one aspect, the subject polymer compositions may be infiltrated into tissue adjacent to a peripheral bypass surgery site. The subject polymer compositions may contain a therapeutic agent (e.g., an anti-scarring and/or anti-infective agent).

In one aspect, the subject polymer compositions may be infiltrated into tissue adjacent to an anastomotic closure device. The subject polymer compositions may contain a therapeutic agent (e.g., an anti-scarring and/or anti-infective agent).

In one aspect, the subject polymer compositions may be infiltrated into tissue adjacent to a transplant surgery site. The subject polymer compositions may contain a therapeutic agent (e.g., an anti-scarring and/or anti-infective agent).

According to the one aspect, any anti-scarring agent described above may be utilized in the practice of the present invention. In one aspect of the invention, the subject polymer compositions infiltrated into tissue adjacent to films and meshes may be adapted to contain and/or release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue).

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As films and meshes are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm²to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

Glaucoma Drainage Devices

In one aspect, the subject polymer compositions may be infiltrated into tissue adjacent to a glaucoma drainage device. The subject polymer compositions may contain a therapeutic agent (e.g., an anti-scarring and/or anti-infective agent).

Various types of glaucoma drainage devices may be used in the practice of this aspect. Some glaucoma drainage devices include a plate and a tube. The function of the tube is to deliver aqueous from within the eye onto the upper surface of the episcleral plate. The episcleral plate is firmly sutured to the sclera and covered by a thick flap of Tenon's tissue and conjunctiva. The function of the plate is to initiate the formation of a large circular bleb which develops a specialized fibrovascular bleb lining and becomes distended by aqueous. It is this fibrovascular bleb lining which is responsible for regulating the escape of aqueous from the eye and which determines the final level of intraocular pressure (IOP) that is achieved after insertion of the implant. If the fibrovascular response is too great, the drainage capability of the device is reduced. In one aspect of the present invention, a polymer composition that includes a fibrosis-inhibiting agent is infiltrated into tissue adjacent to all or a portion of the device such that the released fibrosis-inhibiting agent modulates the healing response, thereby enabling the device to function correctly. In another aspect of the present invention, a polymer composition that includes an anti-infective agent (either alone or in conjunction with a fibrosis-inhibiting agent) is infiltrated into tissue adjacent to all or a portion of the device such that the released anti-infective agent inhibits or prevents infection.

Glaucoma drainage devices may be, for example, a conduit attached to an episcleral drainage plate having a porous posterior surface for cellular ingrowth and attachment by the sclera. See, e.g., U.S. Pat. No. 5,882,327. The glaucoma drainage device may be composed of a foldable and rollable episcleral plate and a drainage tube whereby the device may be delivered to the implant site through an injection delivery system. See, e.g., U.S. Pat. No. 6,589,203. The glaucoma drainage device may be pressure regulator composed of a base plate formed of a thin, flexible rubber material (e.g., silicone rubber) which has a mounted housing chamber that is attached to a tube. See, e.g., U.S. Pat. No. 5,752,928. The glaucoma drainage device may be composed of an elastomeric plate having a sealing member that conforms to the sclera to restrict fluid and an attached non-valved elastomeric drainage tube. See, e.g., U.S. Pat. No. 5,476,445. The glaucoma drainage device may be composed of ridged plates that extend outwardly that are concave on one side to match the curvature of the sclera and are adapted for side by side attachment to the sclera whereby a tube extends between the ridged plates for communication. See, e.g., U.S. Pat. No. 4,457,757. The glaucoma drainage device may be composed of a thin, elliptical, elastomeric plate having a centrally positioned hole for growth of scar tissue and an elastomeric drainage tube attached to the plate for fluid communication with the eye. See, e.g., U.S. Pat. No. 5,397,300. The glaucoma drainage device may be composed of a tube with a circumferential hole with a connected disk at the outlet end of the tube for placing on a surface of an eyeball. See, e.g., U.S. Pat. No. 5,868,697. The glaucoma drainage device may be a tube with a flow controlling structure that constricts flow passage within the tube and has at least one circumferential hole within the tube that is temporarily occluded with an absorbable material. See, e.g., U.S. Pat. No. 6,203,513. The glaucoma drainage device may be composed of a tube with an engagement means and a porous, liquid-absorbing plug with an attached filamentary extension that substantially restricts fluid flow. See, e.g., U.S. Pat. No. 5,300,020. The glaucoma drainage device may be a resilient polymeric drain implant with a passage extending between the ends and flanges that project radially from the body. See, e.g., U.S. Pat. No. 4,968,296. The glaucoma drainage device may be a shunt to divert aqueous humor in the eye from the anterior chamber into a portion of the device that branches to provide fluid communication in either direction along the Schlemm's canal. See, e.g., U.S. Pat. No. 6,626,858.

Glaucoma drainage devices, which may benefit from having the subject polymer composition infiltrated into adjacent tissue according to the present invention, include commercially available products. For example, cylindrical tubes, such as the AQUAFLOW Collagen Glaucoma Drainage Device (STAAR Surgical Company, Monrovia, Calif.) may be used in the practice of the present invention. Other examples of glaucoma drainage devices includes the Molteno Glaucoma Implant (Single Plate Molteno Implant, Pressure Ridge Single Plate Molteno Implant (D1), Microphthalmic Plate Molteno Implant (M1), Double Plate Molteno Implant (R2/L2), and Pressure Ridge Double Plate Molteno Implant (DR2/DL2) from Molteno Opthalmic Limited (New Zealand), BAERVELDT Glaucoma Implants (Models BG-101-350, BG-102-350, BG-103-250; Pfizer, New York, N.Y.), and the Ahmed Glaucoma Valve (Models FP7, S2, S3, PS2, PS3, B1 from New World Medical, Inc. (Rancho Cucamonga, Calif.).

In one aspect, the present invention provides a glaucoma drainage device having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with glaucoma drainage devices have been described above.

Polymeric compositions may be infiltrated around implanted glaucoma drainage devices by applying the composition directly and/or indirectly into and/or onto (a) tissue adjacent to the glaucoma drainage device; (b) the vicinity of the glaucoma drainage device-tissue interface; (c) the region around the glaucoma drainage device; and (d) tissue surrounding the glaucoma drainage device. Methods for infiltrating the subject polymer compositions into tissue adjacent to a glaucoma drainage device include delivering the polymer composition: (a) to the glaucoma drainage device surface (e.g., as an injectable, paste, gel or mesh) during the implantation procedure; (b) to the surface of the tissue (e.g., as an injectable, paste, gel, in situ forming gel or mesh) immediately prior to, or during, implantation of the glaucoma drainage device; (c) to the surface of the glaucoma drainage device and/or the tissue surrounding the implanted glaucoma drainage device (e.g., as an injectable, paste, gel, in situ forming gel or mesh) immediately after the implantation of the glaucoma drainage device; (d) by topical application of the composition into the anatomical space where the glaucoma drainage device may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the glaucoma drainage device as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device.

In one aspect, the methods above can be used to infiltrate the subject polymer composition into tissue adjacent to all or portions of the plate of the device.

In another aspect, the methods above can be used to infiltrate the subject polymer composition into tissue adjacent to all or portions of the tube of the device.

In yet another aspect, the methods above can be used to infiltrate the subject polymer composition into tissue adjacent to all or potions of both the plate and the tube of the device.

According to the present invention, any fibrosis-inhibiting and/or anti-infective agent described above can be utilized in the practice of the present invention. In one aspect of the invention, the subject polymer compositions infiltrated into tissue adjacent to glaucoma drainage devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced. Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As glaucoma drainage devices are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

Prosthetic Heart Valves

In one aspect, the subject polymer compositions may be infiltrated into tissue adjacent to a prosthetic heart valve. The subject polymer compositions may contain a therapeutic agent (e.g., an anti-scarring and/or anti-infective agent).

Prosthetic heart valves are devices that are used to replace natural heart valves that are defective, due to congenital malformations, infections, partial occlusion, or wearing. Prosthetic heart valves are typically composed of an occluder(s) attached to the occluder base, which is in turn attached to the suture ring that provides anchorage of the device to the heart tissue. The occluder base is annular and provides a passageway for blood flow. There may be one or more occluders which alternate in an opened and closed position to regulate the flow of blood. To secure the prosthetic heart valve to the heart tissue, a suture ring, typically composed of a knit fabric tube, is rolled into a toroidal form and is secured to the periphery of the occluder base of the prosthesis. Affixing the suture ring to the heart tissue typically occurs using sutures, sealants, adhesives, staples, or clamping with metal or polymer wires.

Although the design of prosthetic heart valves has been gradually refined, complications continue to occur. Since the suture rings are often made out of synthetic material, thrombus, fibrosis and pannus often occur around the prosthetic heart valve. This scar formation often hinders the function of the valve and over time may require a second surgical procedure and replacement. Suture rings are generally composed of synthetic polymer, including, but not limited to, polyester (e.g., DACRON), polytetrafluoroethylene (e.g., TEFLON), silicone, and polypropylene. Suture rings are often made of a filler material with a woven material stitched over the filler. The surface of the suture ring is often coarse due to the covering cloth material. This predisposes the suture ring to scarring formation early in the post-operative period with severe pannus/fibrosis developing over several months following implantation. The consequences of fibrosis encroachment onto a prosthetic heart valve can be drastic, and potentially catastrophic. For example, fibrosis may inhibit valve occluder function by limiting its ability to open and close properly. The fibrosis may extend from the suture ring to the leaflets. This fibrosis may fuse the leaflets at their commissure, distort individual leaflets, and/or stiffen leaflets such that they do not open or close properly. The end result of this fibrosis typically is a heart valve that is both stenotic and insufficient. Prosthetic heart valves can also be sources of infection in the tissue surrounding the implant site.

There are two main types of prosthetic heart valves, mechanical and bioprosthetic. Typically, both mechanical and bioprosthetic heart valves utilize a synthetic suture ring. They differ primarily in the type of occluder that is utilized. The occluders of the mechanical heart valve may be composed of a ball and cage assembly, single leaflet disk valves, or bileaflet disk valves. The occluders of the bioprosthetic heart valve are composed of animal or human tissue that mimic the appearance and function of the natural heart valve it is replacing. The bioprosthetic heart valve leaflets are usually composed of chemically treated tissue. The harvested valves are fixed in glutaraldehyde or similar fixatives in order to make them suitable for human implantation.

In one aspect, the prosthetic heart valve may be a mechanical prosthesis which is typically composed of rigid leaflets formed of a biocompatible substance (e.g., pyrolitic carbon, titanium or DACRON). Mechanical prosthetic heart valves may be a ball and cage assembly, bileaflet, trileaflet or tilting disks. The most common is the bileaflet type since the hemodynamics of this valve is better as blood flow is smoother and less turbulent. For example, the mechanical prosthesis may be composed of a base with an external suture ring and an internal rim for blood flow as well as at least two closing leaflets. See, e.g., U.S. Pat. No. 6,068,657. The mechanical prosthesis may be composed of annular-valve housing with a center orifice and first and second valve leaflets pivotally mounted to the valve housing. See, e.g., U.S. Pat. Nos. 4,808,180 and 5,026,391. The mechanical prosthesis may be designed with an annular body with at least one leaflet pivotally mounted such that it is movable between an open and closed position by a magnet that exerts a force on the leaflet at a defined pressure. See, e.g., U.S. Pat. No. 6,638,303. The mechanical prosthesis may have an annular body with a plurality of hinges which form an entrance ramp and supports at least one leaflet to the valve body. See, e.g., U.S. Pat. Nos. 6,645,244 and 5,919,226. The mechanical prosthesis may be composed of a supporting flexible, cylindrical frame with a cover that forms a cusp supporting stent for the valve trileaflet apparatus and a sewing ring as an attachment surface. See, e.g., U.S. Pat. No. 5,258,023. The mechanical prosthesis may have an increased valve lumen composed of a single piece valve orifice housing with at least one movable occluder coupled to the housing and a suture cuff for attaching the housing to the heart tissue. See, e.g., U.S. Pat. Nos. 6,007,577 and 6,391,053. The mechanical prosthesis may be composed of a sewing ring and a removable valve assembly which slides in a central core of the sewing ring. See, e.g., U.S. Pat. No. 5,032,128. The mechanical prosthesis may be a highly flexible cylindrical stent composed of a plurality of separate adjacent stent members with alternating cusps and commissures that are able to move radially and support a plurality of flexible leaflets. See, e.g., U.S. Pat. Nos. 6,558,418 and 6,338,740. Other mechanical heart valve prostheses are described in, e.g., U.S. Pat. Nos. 6,395,025; 6,358,278; 6,176,877; 6,139,575 and 5,984,958.

In another aspect, the prosthetic heart valve may be a bioprosthetic device which typically is flexible leaflets formed of a biological material (e.g., porcine valves or bovine pericardial valves). Tissue valves may be supported with a stent frame that provides the leaflets with more structure and durability. Stentless tissue valves may also be implanted by harvesting the porcine valves with the pig's aorta still attached. For example, the bioprosthetic heart valve, which may be obtained from a donor (e.g., porcine), may be treated to reduce antigens to prevent inflammatory response upon transplantation. See, e.g., U.S. Pat. No. 6,592,618. The bioprosthetic heart valve may be composed of a biological tissue material disposed around a mechanical annular support to provide at least part of the sewing ring. See, e.g., U.S. Pat. No. 6,582,464. The bioprosthetic heart valve may be composed of a xenograft mitral valve (e.g., porcine) and a sewing tube and cover of flexible material which is attached to the mitral valve. See, e.g., U.S. Pat. No. 5,662,704. The bioprosthetic heart valve may be composed of a natural tissue heart valve attached to a prosthetic stent frame that may be covered by a fabric cover. See, e.g., U.S. Pat. Nos. 3,983,581; 4,035,849; 5,861,028; 6,350,282 and 6,585,766. The bioprosthetic heart valve may be a self-supporting stentless valve that may be composed of a tubular body of mammalian origin. See, e.g., U.S. Pat. Nos. 5,156,621 and 6,342,070.

In another aspect, the prosthetic heart valve may be inserted into place using minimally-invasive techniques. For example, the prosthetic heart valve may be an expandable device adapted for delivery in a collapsed state to an implantation site and then expanded to a plurality of leaflets attached to a stent system. See, e.g., U.S. Pat. No. 6,454,799.

In another aspect, the device may be a component of the heart valve. For example, the device may be an implantable annular ring for receiving a prosthetic heart valve. See, e.g., U.S. Pat. No. 6,106,550. The device may be a suture ring having an outer peripheral tapered thread for attaching a heart valve prosthesis. See, e.g., U.S. Pat. No. 6,113,632. The device may be a suture ring for a mechanical heart valve composed of a stiffening ring attachment, a knit fabric sewing cuff and a locking ring. See, e.g., U.S. Pat. No. 5,071,431.

Prosthetic heart valves and components thereof (e.g., annular suture rings), which may benefit from having the subject polymer composition infiltrated into adjacent tissue according to the present invention, include commercially available products, such as the Carpentier-Edwards PERIMOUNT (CEP) Pericardial Bioprosthesis, Carpentier-Edwards S.A.V. Aortic Bioprosthesis and Edwards PRIMA PLUS STENTLESS BIOPROSTHESIS from Edwards Lifesciences (Irvine, Calif.), the SJM REGENT Valve from St. Jude Medical (St. Paul, Minn.), and the MOSAIC Bioprosthetic Heart Valve from Medtronic (Minneapolis, Minn.).

In one aspect, the present invention provides prosthetic heart valve devices having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with prosthetic heart valves have been described above.

Polymeric compositions may be infiltrated around implanted prosthetic heart valves by applying the composition directly and/or indirectly into and/or onto (a) tissue adjacent to the prosthetic heart valve; (b) the vicinity of the prosthetic heart valve-tissue interface; (c) the region around the prosthetic heart valve; and (d) tissue surrounding the prosthetic heart valve. Methods for infiltrating the subject polymer compositions into tissue adjacent to a prosthetic heart valve include delivering the polymer composition: (a) to the prosthetic heart valve surface (e.g., as an injectable, paste, gel or mesh) during the implantation procedure; (b) to the surface of the tissue (e.g., as an injectable, paste, gel, in situ forming gel or mesh) immediately prior to, or during, implantation of the prosthetic heart valve; (c) to the surface of the prosthetic heart valve and/or the tissue surrounding the implanted prosthetic heart valve (e.g., as an injectable, paste, gel, in situ forming gel or mesh) immediately after the implantation of the prosthetic heart valve; (d) by topical application of the composition into the anatomical space where the prosthetic heart valve may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the prosthetic heart valve as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device.

In some aspects, the subject polymer compositions may be infiltrated into tissue adjacent to: (a) the surface of the annular ring (particularly mechanical valves); (b) the surface of the valve leaflets (particularly bioprosthetic valves); and/or (c) any combination of the aforementioned.

According to one aspect, any fibrosis-inhibiting and/or anti-infective agent described above may be utilized in the practice of the present invention. In one aspect of the invention, the subject polymer compositions infiltrated into tissue adjacent to prosthetic heart valves may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As prosthetic heart valves are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

Penile Implants In one aspect, the subject polymer compositions may be infiltrated into tissue adjacent to a penile implant device. The subject polymer compositions may contain a therapeutic agent (e.g., an anti-scarring and/or anti-infective agent). In one aspect, the subject polymer compositions infiltrated into tissue adjacent to penile implants are loaded with an anti-scarring drug to prevent fibrous encapsulation. In another aspect, the subject polymer compositions infiltrated into tissue adjacent to penile implants are loaded with an anti-infective agent (either alone or in conjunction with an anti-scarring drug) to prevent fibrous infection and/or encapsulation

Penile implants are used to treat erectile dysfunction and are generally flexible rods, hinged rods or inflatable devices with a pump. Penile implants may be composed of rods, coils, inflatable tubes and/or pressure chambers and may be used to provide erectile function, enlargement or provide shape to a misshapen or damaged penis. For example, the penile implant may be an implantable polymeric material which is injected into the lamina propria mucosae of the glans in order to enlarge the glans of the male genital organ. See, e.g., U.S. Pat. No. 6,418,934. The penile implant may be composed of a pair of arced, elongated portions made of silicone rubber that are mirror images of each other, which has a varying circumferential wall thickness. See, e.g., U.S. Pat. No. 6,537,204. The penile implant may be used to increase penile volume by being adapted to cover the outer lateral sides of the corpus cavernosum without covering the upper and lower sides thereof. See, e.g., U.S. Pat. No. 6,015,380. The penile implant may be an inflatable, self-contained implant composed of a cylindrical body having a pump that transfers fluid from a reservoir to a pressure chamber that has a pressure relief valve. See, e.g., U.S. Pat. Nos. 4,898,158 and 4,823,779. The penile implant may be composed of an elongated rod having a relatively short proximal stem portion, which is covered by a layer of hydrophilic material that contains a plurality of openings and swells as it absorbs water. See, e.g., U.S. Pat. No. 4,611,584. The penile implant may be composed of at least one inflatable tube that has fluid interchange with a mounting base which is controlled by a manual pump implanted in the scrotum. See, e.g., U.S. Pat. No. 6,475,137. The penile implant may be a flexible double-walled partial cylindrical sleeve that has bellow-like construction which is suited for penile malformation. See, e.g., U.S. Pat. No. 5,669,870. The penile implant may be used for correcting erectile impotence by being composed of at least one flexible portion with a pressure chamber connected by tubing to an accumulator charged with fluid, such that pressurizing fluid flows when the valve is opened. See, e.g., U.S. Pat. No. 4,917,110. The penile implant may be composed of a stainless steel pad supported by a plurality of strands which is surrounded by a cylinder with a silicone ring that can move longitudinally in response to the expansion or shrinkage of the penis. See, e.g., U.S. Pat. No. 5,433,694. The penile implant may increase girth and length by being composed of a cylindrical sleeve that has an elastic outer sheet and an inner inelastic sheet that forms a closed sack to receive a fluid under pressure from a fluid source. See, e.g., U.S. Pat. No. 5,445,594. The penile implant may be composed of a braided sleeve with an outer elastomeric surface and inner surface having grooves and ribs in a helical arrangement, such that the implant is malleable having both a bendable configuration and an unbent rigid configuration. See, e.g., U.S. Pat. No. 5,512,033. The penile implant may be a polymeric matrix having dissociated cartilage-forming cells deposited on and in said matrix whereby a cartilaginous structure is formed upon implantation having controlled biomechanical properties and tensile strength. See, e.g., U.S. Pat. No. 6,547,719. The penile implant may be composed of an implantable supply pump, deformable reservoir, and conducting/dispensing catheters, such that a vasodilator agent is delivered to the erectile bodies to treat male impotence. See, e.g., U.S. Pat. No. 6,679,832. Other penile implants are described in, e.g., U.S. Pat. Nos. 6,579,230; 5,704,895; 5,250,020; 5,048,510 and 4,875,472.

Penile implants, which may benefit from having the subject polymer composition infiltrated into adjacent tissue according to the present invention, include commercially available products, such as, for example, the TITAN Inflatable Penile Prosthesis from Mentor Corporation (Santa Barbara, Calif.) and the AMS penile prosthesis product line including the AMS 700 CX CXM, AMS AMBICOR, and AMS Malleable 600M Penile Prostheses from American Medical Systems, Inc. (Minnetonka, Minn.), In one aspect, the present invention provides penile implant devices having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with penile implants have been described above.

Polymeric compositions may be infiltrated around implanted penile implants by applying the composition directly and/or indirectly into and/or onto (a) tissue adjacent to the penile implant; (b) the vicinity of the penile implant-tissue interface; (c) the region around the penile implant; and (d) tissue surrounding the penile implant. Methods for infiltrating the subject polymer compositions into tissue adjacent to a penile implant include delivering the polymer composition: (a) to the penile implant surface (e.g., as an injectable, paste, gel or mesh) during the implantation procedure; (b) to the surface of the tissue (e.g., as an injectable, paste, gel, in situ forming gel or mesh) immediately prior to, or during, implantation of the penile implant; (c) to the surface of the penile implant and/or the tissue surrounding the implanted penile implant (e.g., as an injectable, paste, gel, in situ forming gel or mesh) immediately after the implantation of the penile implant; (d) by topical application of the composition into the anatomical space where the penile implant may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the penile implant as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device.

The placement of penile implants can be complicated by infection (usually in the first 6 months after surgery) with Coagulase Negative Staphylococci (including Staphylococcus epidermidis), Staphylococcus aureus, Pseudomonas aeruginosa, Enterococci, Serratia and Candida. Infection is characterized by fever, erythema, induration and purulent drainage from the operative site. The usual route of infection is through the incision at the time of surgery and up to 3% of penile implants become infected despite the best sterile surgical technique. To help combat this, intraoperative irrigation with antibiotic solutions is often employed.

Infiltrating into the tissue adjacent to the penile implant a polymer composition containing an anti-infective agent can allow bacteriocidal drug levels to be achieved locally, thus reducing the incidence of bacterial colonization (and subsequent development of local infection and device failure), while producing negligible systemic exposure to the drugs.

According to the one aspect, any fibrosis-inhibiting and/or anti-infective agent described above may be utilized in the practice of the present invention. In one aspect of the invention, the subject polymer compositions infiltrated into tissue adjacent to penile implants may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As penile implants are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

Endotracheal and Tracheostomy Tubes

In one aspect, the subject polymer compositions may be infiltrated into tissue adjacent to endotracheal and tracheostomy tube devices. The subject polymer compositions may contain a therapeutic agent (e.g., an anti-scarring and/or anti-infective agent). Association of an anti-scarring agent with an endotracheal or a tracheostomy tube (e.g., chest tube), or adjacent tissue, may be used to prevent stenosis and/or infection of the artificial airway.

Endotracheal tubes and tracheostomy tubes are used to maintain the airway when ventilatory assistance is required. Endotracheal tubes tend to be used to establish an airway in the acute setting, while tracheostomy tubes are used when prolonged ventilation is required or when there is a fixed obstruction in the upper airway.

In one aspect, endotracheal tubes may be used to provide a mechanical air passageway, which may be required for ventilation of the lungs during injury or surgery. Endotracheal tubes may have a single lumen or double lumen, and may have a flange or balloon for engaging its position within the trachea. For example, the endotracheal tube may be composed of an inner and outer flexible tube having a radially extending flange that prevents advancement beyond the larynx. See, e.g., U.S. Pat. No. 5,259,371. The endotracheal tube may have a double lumen which is removably affixed whereby the first tubular lumen may be removed from the airway while the second tubular lumen remains intact. See, e.g., U.S. Pat. No. 6,443,156. The endotracheal tube may have a tracheal portion and a bronchial portion attached at an angle that forms a single lumen, whereby when a balloon that is positioned within the tube is inflated, it blocks the flow of gas through the bronchial portion. See, e.g., U.S. Pat. No. 6,609,521. The endotracheal tube may be composed of two cylindrical portions of different diameters which are connected by a non-circularly shaped tapered portion to complement the glottis which has a plurality of sealing gills that are thin and pliable that extends from the tapered portion. See, e.g., U.S. Pat. No. 5,429,127. The endotracheal tube may be composed of a tubular portion with a visual indicator to provide guidance of the rotational orientation of the beveled tip at the distal end as it is advanced along the airway. See, e.g., U.S. Pat. No. 6,568,393. The endotracheal tube may be composed of a light reflective coated bore to enhance image transmission and a flexible plurality of passages, one adapted to receive a fiber optic bundle, another connected to an inflatable cuff, and another adapted to receive a malleable stylette to aid in insertion and removal. See, e.g., U.S. Pat. No. 6,629,924. The endotracheal tube may be composed of a hollow, flexible, cylindrical tube having an annular flange at its tip and a connector with an annular internal ridge that is concentrically mounted upon the outer proximal surface of the tube portion. See, e.g., U.S. Pat. No. 5,251,617. The endotracheal tube may be composed of a main tube with an inflatable cuff for sealing, which has a double lumen for irrigation and suction for removal of secretions that may pool in the trachea. See, e.g., U.S. Pat. No. 5,143,062. Other endotracheal tubes are described in, e.g., U.S. Pat. Nos. 6,321,749; 5,765,559; 5,353,787; 5,291,882 and 4,977,894.

Tracheostomy tubes can be used to provide a bypass supply of air when the throat is obstructed. Tracheostomy tubes are used with an obturator for percutaneous insertion into a trachea through a stoma in the neck between adjacent cartilages to assist breathing. For example, the tracheostomy tube may be a tubular cannula formed of soft flexible plastic material which has a tapered distal end that is beveled, narrow, angled and curved downwardly for positioning within the trachea. See, e.g., U.S. Pat. No. 5,058,580. The tracheostomy tube may be composed of a tube with a removable fitting mounted on the exposed end which may be sealed to the tube. See, e.g., U.S. Pat. No. 5,606,966. The tracheostomy tube may be composed of an arcuate cannula with a flange that extends laterally outward and a rotatable tubular elbow that has a fluid connection with the cannula. See, e.g., U.S. Pat. Nos. 5,259,376 and 5,054,482. The tracheostomy tube may be composed of two airways with a pneumatic vibrator that generates sonic vibrations to permit audible speech. See, e.g., U.S. Pat. No. 4,773,412. The tracheostomy tube may be composed of an inner cannula removably received within an outer cannula with a sealing cuff between the outer cannula and the trachea to substantially prevent air from escaping from the trachea and to allow phonation through a secondary passageway formed between the inner and outer cannula. See, e.g., U.S. Pat. No. 4,573,460. The tracheostomy tube may be composed of a first port for orienting outside the neck of the wearer, a second port for orienting within the trachea, and a third connecting port to provide and control gas flow via a valve. See, e.g., U.S. Pat. No. 5,957,978. The tracheostomy tube may be composed of a hollow tube, an inflatable balloon having orthogonal projections, and a flange that provides an anchor external to the throat. See, e.g., U.S. Pat. No. 6,612,305. The tracheostomy tube may be composed of a highly flexible material having wire reinforcement and a neck plate with a collar portion that may slide along the tube. See, e.g., U.S. Pat. No. 5,443,064. Other tracheostomy tubes are described in, e.g., U.S. Pat. Nos. 6,662,804; 6,135,110 and 5,983,895.

Endotracheal tubes, which may benefit from having the subject polymer composition infiltrated into adjacent tissue according to the present invention, include commercially available products, such as the HI-LO Tracheal Tubes, LASER-FLEX Tracheal Tubes, and ENDOTROL Tracheal Tubes from Nellcor Puritan Bennett Inc. (Pleasanton, Calif.), the SHERIDAN Endotracheal Tubes from Hudson RCI (Temecula, Calif.), and the BARD Endotracheal Tube, Cuffed from C.R. Bard, Inc. (Murray Hill, N.J.).

Tracheostomy tubes, which may benefit from having the subject polymer composition infiltrated into adjacent tissue according to the present invention, include commercially available products, such as the SHILEY TRACHEOSOFT XLT Tracheostomy Tubes, PHONATE Speaking Valves, and Reusable Cannula Cuffless Tracheostomy Tubes from Nellcor Puritan Bennett Inc. (Pleasanton, Calif.), the PER-FIT Percutaneous Dilational Tracheostomy Kits, PORTEX BLUE LINE Cuffed Tracheostomy Tubes, and BIVONA Uncuffed Tracheostomy Tubes from Portex, Inc. (Keene, N.H.), and the CRYSTALCLEAR Tracheostomy Tubes from Rusch (Germany).

In one aspect, the present invention provides endotracheal and tracheostomy tube devices having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with endotracheal and tracheostomy tube devices have been described above.

Polymeric compositions may be infiltrated around implanted endotracheal and tracheostomy tube devices by applying the composition directly and/or indirectly into and/or onto (a) tissue adjacent to the endotracheal or tracheostomy tube device; (b) the vicinity of the endotracheal or tracheostomy tube device-tissue interface; (c) the region around the endotracheal or tracheostomy tube device; and (d) tissue surrounding the endotracheal or tracheostomy tube device. Methods for infiltrating the subject polymer compositions into tissue adjacent to endotracheal or tracheostomy tube devices include delivering the polymer composition: (a) to the endotracheal or tracheostomy tube device surface (e.g., as an injectable, paste, gel or mesh) during the implantation procedure; (b) to the surface of the tissue (e.g., as an injectable, paste, gel, in situ forming gel or mesh) immediately prior to, or during, implantation of the endotracheal or tracheostomy tube device; (c) to the surface of the endotracheal or tracheostomy tube device and/or the tissue surrounding the implanted endotracheal or tracheostomy tube device (e.g., as an injectable, paste, gel, in situ forming gel or mesh) immediately after the implantation of the endotracheal or tracheostomy tube device; (d) by topical application of the composition into the anatomical space where the endotracheal or tracheostomy tube device may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the endotracheal or tracheostomy tube device as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device.

According to one aspect, any fibrosis-inhibiting and/or anti-infective agent described above may be utilized in the practice of the present invention. In one aspect of the invention, the subject polymer compositions infiltrated into tissue adjacent to endotracheal and tracheostomy tube devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As endotracheal and tracheostomy tube devices are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

Peritoneal Dialysis Catheters

In one aspect, the subject polymer compositions may be infiltrated into tissue adjacent to a peritoneal dialysis catheter or a peritoneal implant for drug delivery. The subject polymer compositions may contain a therapeutic agent (e.g., an anti-scarring and/or anti-infective agent).

Peritoneal dialysis catheters are typically double-cuffed and tunneled catheters that provide access to the peritoneum. The most common peritoneal dialysis catheter designs are the Tenckhoff catheter, the Swan Neck Missouri catheter and the Toronto Western catheter. In peritoneal dialysis, the peritoneum acts as a semipermeable membrane across which solutes can be exchanged down a concentration gradient. Continuous peritoneal access catheters are permanently implanted for those that require repeated access to the peritoneum. Implanted peritoneal catheters may be used for peritoneal dialysis or for a means of delivering drug to the peritoneum. These catheters may be composed of synthetic materials, such as silicone, rubber, polyurethane or other polymers that provide flexibility. They may be designed to be configured as a straight tube or may be bent and molded into a variety of shapes to provide different configurations, including helices and coils. The peritoneal catheters may be composed of one continuous element or may be sectioned into parts to provide flanges, cuffs, beads or discs at one of the ends to fix the catheter in position.

For example, the peritoneal catheter may be a resilient, foldable, T-shaped housing chamber with access ports that have elongated, flexible, fluid channels that gather or distribute a liquid such as dialysis fluid. See, e.g., U.S. Pat. No. 5,322,519. The peritoneal catheter may be composed of two linearly mated inflow and outflow conduits contoured as a circular cross-section, which join fluted fluid transport branches. See, e.g., U.S. Pat. No. 6,659,134. The peritoneal catheter may be composed of a ductwork of multiple tubes with fluid holes enclosed within a fluid permeable envelope structure that has slits to allow fluid flow but not tissue adherence. See, e.g., U.S. Pat. No. 5,254,084. The peritoneal catheter may have a one-half helical turn to provide a radial flow and be composed of a plurality of ingress and egress ports positioned about its circumference and length, and have a coating of ultra low temperature isotropic carbon on the intra-abdominal section. See, e.g., U.S. Pat. No. 5,098,413. The peritoneal catheter may be an elongated flexible tube with one end connected to a pair of spaced apart sheets that extends exteriorly into the body cavity with at least one cuff for preventing catheter infections. See, e.g., U.S. Pat. No. 4,368,737. The peritoneal catheter may be composed of two sections which includes a retainer section that permanently ingrows into the abdominal wall and an elongated flexible tube section for delivering and withdrawing dialysate. See, e.g., U.S. Pat. No. 4,278,092. The peritoneal catheter may be flexible tube having a natural bent segment between the proximal and distal ends which includes a flange extending circumferentially at a nonperpendicular angle relative to the axis of the catheter tube. See, e.g., U.S. Pat. No. 4,687,471. The peritoneal catheter may be a percutaneous access device composed of a cylindrical neck portion for skin protrusion, an annular skirt portion for anchoring into the dermis/subcutaneous tissue, and a catheter tube that may be threaded through the neck and skirt portions that has flexible bellows which can form a 90 degree angle. See, e.g., U.S. Pat. No. 4,886,502. The peritoneal catheter may be a flexible, elongated tube with perforations in the wall to pass fluid with a means for urging the central portion of the tube into a tightly wound cylindrical helix configuration. See, e.g., U.S. Pat. No. 4,681,570. Other examples of peritoneal catheters used for dialysis are described in, e.g., U.S. Pat. Nos. 6,290,669; 5,752,939 and 5,171,227.

In another aspect, the peritoneal catheter may be used to administer drugs to the peritoneum. For example, the peritoneal catheter may be a subcutaneous injection catheter apparatus having a receiving chamber with a penetrable membrane to accommodate an injection needle, which may be interconnected to the peritoneal cavity by a hollow stem. See, e.g., U.S. Pat. No. 4,400,169. The peritoneal catheter may be composed of a porous outer casing defining an inner space with an inlet and outlet catheter of non-porous material which are in communication with an opening of the outer casing to form two passageways. See, e.g., U.S. Pat. No. 5,100,392.

Long-term use of peritoneal catheters may lead to infections or blockage of the catheter due to fibrin formation. Synthetic peritoneal catheters and delivery devices having the subject polymer composition that contains an anti-scarring agent incorporated into adjacent tissue are capable of preventing stenosis. Synthetic peritoneal catheters and delivery devices having the subject polymer composition that contains an anti-infective agent incorporated into adjacent tissue are capable of preventing or inhibiting infection.

Peritoneal catheters, which may from having the subject polymer composition infiltrated into adjacent tissue according to the present invention, include commercially available products. For example, Cook Critical Care (Bloomington, Ind.) sells the Spiral Chronic Peritoneal Dialysis Catheters and Tenckhoff Chronic Peritoneal Dialysis Catheters. Bard Access Systems (Salt Lake City, Utah) sells the Tenckhoff and HEMOSPLIT Peritoneal Dialysis Catheters. CardioMed Supplies, Inc (ON, Canada) sells the Single Cuff and Double Cuff Straight Peritoneal Dialysis Catheters, as well as the Single Cuff and Double Cuff Coiled Peritoneal Dialysis Catheters. Other companies that sell Single and Double Cuff, Straight and Coiled Tenckhoff catheters and other types of peritoneal catheters include Baxter International, Inc. (Deerfield, Ill.), Fresenius Medical Care (Lexington, Mass.) and Gambro AB (Sweden).

In one aspect, the present invention provides peritoneal access catheters and implants having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with peritoneal dialysis implants and catheters have been described above.

Polymeric compositions may be infiltrated around implanted peritoneal access catheters and implants by applying the composition directly and/or indirectly into and/or onto (a) tissue adjacent to the peritoneal access catheter or implant; (b) the vicinity of the peritoneal access catheter or implant-tissue interface; (c) the region around the peritoneal access catheter or implant; and (d) tissue surrounding the peritoneal access catheter or implant. Methods for infiltrating the subject polymer compositions into tissue adjacent to a peritoneal access catheter or implant include delivering the polymer composition: (a) to the peritoneal access catheter or implant surface (e.g., as an injectable, paste, gel or mesh) during the implantation procedure; (b) to the surface of the tissue (e.g., as an injectable, paste, gel, in situ forming gel or mesh) immediately prior to, or during, implantation of the peritoneal access catheter or implant; (c) to the surface of the peritoneal access catheter or implant and/or the tissue surrounding the implanted peritoneal access catheter or implant (e.g., as an injectable, paste, gel, in situ forming gel or mesh) immediately after the implantation of the peritoneal access catheter or implant; (d) by topical application of the composition into the anatomical space where the peritoneal access catheter or implant may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the peritoneal access catheter or implant as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device.

According to one aspect, any fibrosis-inhibiting and/or anti-infective agent described above may be utilized in the practice of the present invention. In one aspect of the invention, the subject polymer compositions infiltrated into tissue adjacent to peritoneal dialysis implants and catheters may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As peritoneal dialysis implants and catheters are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²- 2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²- 10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

Central Nervous System Shunts and Pressure Monitoring Devices

In one aspect, the subject polymer compositions may be infiltrated into tissue adjacent to a central nervous system (CNS) device, such as a CNS shunt or a pressure monitoring device. The subject polymer compositions may contain a therapeutic agent (e.g., an anti-scarring and/or anti-infective agent). CNS devices having the subject polymer composition comprising an anti-scarring agent infiltrated into adjacent tissue are capable of preventing stenosis and obstruction of the device leading to hydrocephalus and increased intercranial pressure. CNS devices having the subject polymer composition comprising an anti-infective agent infiltrated into adjacent tissue are capable of preventing or inhibiting infection in the tissue surrounding the device.

Hydocephalus, or accumulation of cerebrospinal fluid (CSF) in the brain, is a frequently encountered neurosurgical condition arising from congenital malformations, infection, hemorrhage, or malignancy. The incompressible fluid exerts pressure on the brain leading to brain damage or even death if untreated. CNS shunts are conduits placed in the ventricles of the brain to divert the flow of CSF from the brain to other body compartments and relieve the fluid pressure. Ventricular CSF is diverted via a prosthetic shunt to a number of drainage locations including the pleura (ventriculopleural shunt), jugular vein, vena cava (VA shunt), gallbladder and peritoneum (VP shunt; most common).

Representative examples of CNS devices include, e.g., CNS shunts, such as ventriculopleural shunts, jugular vein and vena cava (VA) shunts, and ventriculoperitoneal shunt (VP shunt), such as gallbladder and peritoneum shunts; External Ventricular Drainage (EVD) devices; and Intracranial Pressure (ICP) Monitoring Devices. Other CNS devices include, e.g., dural patches and implants to prevent epidural fibrosis post-laminectomy; and devices for continuous subarachnoid infusions.

In one aspect, the CNS device may be a drainage shunt used to drain fluids in the brain. For example, the CNS device may be a cerebrospinal shunt composed of two tubes whereby an inner tube supplies the fluid from the brain ventricles to the peritoneum region and an outer tube is arranged to exert pressure on the inner tube as the volume of fluid builds in the outer tube. See, e.g., U.S. Pat. No. 5,405,316. The CNS device may be a ventricular drainage system adapted for connection to a ventricular drainage catheter for receiving cerebrospinal fluid and having a valve for controlling fluid flow therethrough. See, e.g., U.S. Pat. No. 5,772,625. The CNS device may be a brain ventricular shunt system composed of a brain check valve for preventing cerebrospinal fluid backflow and a flow-rate switching mechanism to provide flow of cerebrospinal fluid from the brain ventricle catheter to the peritoneum or auricle catheter. See, e.g., U.S. Pat. No. 4,781,673. The CNS device may be shunt member with a flow restricting passage that is connected to catheters to provide cerebrospinal fluid drainage from the brain ventricle to the sinus sagittalis. See, e.g., U.S. Pat. No. 6,283,934. The CNS device may be a ventricular end of a ventriculo-cardiac shunt that has a closed distal end with lateral passageways adjacent thereto which are porous and expansible for providing an umbrella-like liner to allow passage of fluid while preventing obstruction. See, e.g., U.S. Pat. No. 3,690,323. The CNS device may be a hydrocephalus valve composed of a chamber with an inlet and outlet valve for routing cerebrospinal fluid away from the brain at a controlled pressure. See, e.g., U.S. Pat. No. 5,069,663. The CNS device may be a hydrocephalus device composed of an external, flexible shell forming a fluid reservoir and housing a non-obstructive, self-regulating valve having a folded membrane which forms a slit-like opening, which has inlet and outlet tubes. See, e.g., U.S. Pat. No. 5,728,061. The CNS device may be a cerebral spinal fluid draining shunt composed of an implantable master control unit that interconnects a cerebral spinal space catheter with a catheter that drains the fluid into a body cavity. See, e.g., U.S. Pat. No. 6,585,677. The CNS device may be a cerebrospinal fluid shunt composed of a ventricular catheter connected to a flexible drainage tube which has an exterior flexible tubular cover from which the drainage tube may be drawn. See, e.g., U.S. Pat. No. 4,950,232. The CNS device may be an intracranial shunting tube composed of a thin film that extends radially and outwardly from the open end of a ventricular tube which has a plurality of side holes to bypass ventricular cerebrospinal fluid to the subdural space on the surface of the brain. See, e.g., U.S. Pat. No. 5,000,731. Other CNS shunts are described in, e.g., U.S. Pat. Nos. 6,575,928; 5,437,626 and 4,631,051.

In another aspect, the CNS device may be a pressure monitoring device. For example, the pressure monitoring device may be an intracranial pressure sensor which is mounted within the skull of a body at the situs where the pressure is to be monitored and a means of transmitting the pressure externally from the skull. See, e.g., U.S. Pat. No. 4,003,141. The pressure monitoring device may be a telemetric differential pressure sensitive device composed of a thin, planar, closed, conductive loop which moves with a flexible diaphragm upon changes in the difference of two bodily pressures on its opposite sides. See, e.g., U.S. Pat. No. 4,593,703. The pressure monitoring device may be composed of a radio-opaque liquid contained within a resiliently compressible vessel of a silastic material in which the volume of liquid is variable as a function of the pressure or force applied to the vessel. See, e.g., U.S. Pat. No. 3,877,137. The pressure monitoring device may be a probe composed of a threaded shaft having a lumen and an engaging lock nut, which is inserted through an opening in the scalp and into the subarachnoid space. See, e.g., U.S. Pat. No. 4,600,013. The pressure monitoring device may be composed of an external transceiver unit and an implantable cavity resonator unit having a dielectric-filled cavity with a predetermined resonance frequency for high frequency electromagnetic waves. See, e.g., U.S. Pat. No. 5,873,840. The pressure monitoring device may be an implantable sensor that detects a physiological parameter (e.g., cerebral spinal fluid flow) and then generates, processes, and transmits the signal to an external receiver. See, e.g., U.S. Pat. No. 6,533,733. Other CNS pressure monitoring devices are described in, e.g., U.S. Pat. Nos. 6,248,080 and 6,210,346.

CNS shunts, which may benefit from having the subject polymer composition infiltrated into adjacent tissue according to the present invention, include commercially available products, such as the Codman HAKIM Programmable Valves from Codman & Shurtleff, Inc. (Raynham, Mass.), a Johnson & Johnson Company. Other examples include the Integra Neuro Sciences (Plainsboro, N.J.) HEYER-SCHULTE Neurosurgical Shunts, HERMETIC CSF Drainage Systems, and OSV II SMART VALVE Systems and the Medtronic, Inc. (Minneapolis, Minn.) Shunt Assemblies, including the STRATA, DELTA, CSF-Snap and CSF-Flow Control Shunt Assemblies.

Pressure Monitoring CNS devices, which may benefit from having the subject polymer composition infiltrated into adjacent tissue according to the present invention, include commercially available products such as the VENTRIX Pressure Monitoring Kits and CAMINO Micro Ventricular Bolt ICP Monitoring Catheters from Integra Neuro Sciences (Plainsboro, N.J.).

In one aspect, the present invention provides CNS devices having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with CNS devices have been described above.

Polymeric compositions may be infiltrated around implanted CNS devices by applying the composition directly and/or indirectly into and/or onto (a) tissue adjacent to the CNS device; (b) the vicinity of the CNS device-tissue interface; (c) the region around the CNS device; and (d) tissue surrounding the CNS device. Methods for infiltrating the subject polymer compositions into tissue adjacent to a CNS device include delivering the polymer composition: (a) to the CNS device surface (e.g., as an injectable, paste, gel or mesh) during the implantation procedure; (b) to the surface of the tissue (e.g., as an injectable, paste, gel, in situ forming gel or mesh) immediately prior to, or during, implantation of the CNS device; (c) to the surface of the CNS device and/or the tissue surrounding the implanted CNS device (e.g., as an injectable, paste, gel, in situ forming gel or mesh) immediately after the implantation of the CNS device; (d) by topical application of the composition into the anatomical space where the CNS device may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the CNS device as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device.

According to one aspect, any fibrosis-inhibiting and/or anti-infective agent described above may be utilized in the practice of the present invention. In one aspect of the invention, the subject polymer compositions infiltrated into tissue adjacent to CNS devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As CNS devices are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

Inferior Vena Cava Filters

In one aspect, the subject polymer compositions may be infiltrated into tissue adjacent to an inferior vena cava filter device. The subject polymer compositions may contain a therapeutic agent (e.g., an anti-scarring and/or anti-infective agent). The term inferior vena cava filters are devices that are intended to capture emboli and prevent them from migrating through the blood stream. Examples of inferior vena cava filters include, without limitation, vascular filters, blood filters, implantable blood filters, caval filters, vena cava filters, vena cava filtering devices, thrombosis filters, thrombus filters, antimigration filters, filtering devices, percutaneous filter systems, intravascular traps, intravascular filters, clot filters, vein filters and body vessel filters.

Inferior vena cava filters catch blood clots to prevent them from traveling to other parts of the body to form an embolus. It may be life threatening if plaques or blood clots migrate through the blood stream and travel to the lungs and cause a pulmonary embolism. To prevent such an occurrence, inferior vena cava filters are placed in the large veins of the body to prevent pulmonary emboli in patients with (or at risk of developing) deep vein thrombosis. Most often these filters are composed of synthetic polymers or metals. These filters may be a variety of configurations, including but not limited to, baskets, cones, umbrellas or loops. The shape of the filter must provide adequate trapping ability while allowing sufficient blood flow. Along with the functional shape, filters may also have other design features including peripheral loops for alignment or anchoring features to prevent migration (e.g., ridges, struts or sharp points). Where the filter comes into contact with the vessel wall for anchoring, a fibrotic response may occur. This fibrotic response can result in difficulties in removal of the filter. This is a particular problem for filters that are to be kept in place for a relatively short period of time. The filter can also become a site for infection. Infiltration of a polymer composition containing a fibrosis-inhibiting and/or anti-infective agent into tissue adjacent to the filter may reduce or prevent stenosis or obstruction of the device via a fibroproliferative response and/or may prevent or inhibit infection at the site of the filter.

In one aspect, inferior vena cava filters may be designed in a variety of configurations. For example, the inferior vena cava filter may be composed of a plurality of intraluminal filter elements held by a retainer in a filter configuration that may be released to an open, stent-like configuration. See, e.g., U.S. Pat. No. 6,267,776. The inferior vena cava filter may be composed of an embolus capturing portion having a plurality of elongated filter wires diverging in a helical arrangement to form a conical surface and an anchoring portion that has a plurality of struts. See, e.g., U.S. Pat. No. 6,391,045. The inferior vena cava filter may be composed of a textured echogenic feature so the filter position may be determined by sonographic visualization. See, e.g., U.S. Pat. No. 6,436,120. The inferior vena cava filter may be composed of a plurality of core wire struts that are anchored to radiate outwardly which are interconnected by compression material to form a filter basket. See, e.g., U.S. Pat. No. 5,370,657. The inferior vena cava filter may be composed of an apical head with a plurality of divergent legs in a conical shaped geometry which have a hook and pad for securing to the vessel. See, e.g., U.S. Pat. No. 5,059,205. The inferior vena cava filter may be composed of a filtering device made of shape memory/superelastic material formed at the distal end of a deployment/retrieval wire section for minimally invasive positioning. See, e.g., U.S. Pat. No. 5,893,869. The inferior vena cava filter may be composed of a plurality of intraluminal elements joined by a retainer, whereby upon release of the retainer, the intraluminal filter elements convert to an open configuration in the blood vessel. See, e.g., U.S. Pat. Nos. 6,517,559 and 6,267,776. The inferior vena cava filter may be composed of an outer catheter and an inner catheter having a collapsible mesh-like filter basket at the distal end made of spring wires or plastic monofilaments. See, e.g., U.S. Pat. No. 5,549,626. The inferior vena cava filter may be composed of a plurality of radiating struts that attach at a body element and has a two layer surface treatment to provide endothelial cell growth and anti-proliferative properties. See, e.g., U.S. Pat. No. 6,273,901. The inferior vena cava filter may be composed of a metal fabric that is configured as a particle-trapping screen that may be slideable along a guidewire. See, e.g., U.S. Pat. No. 6,605,102. The inferior vena cava filter may be non-permanent with a single high memory coiled wire having a cylindrical and a conical segment. See, e.g., U.S. Pat. No. 6,059,825. Other inferior vena cava filters are described in, e.g., U.S. Pat. Nos. 6,623,506; 6,391,044; 6,231,589; 5,984,947; 5,695,518 and 4,817,600.

Vena cava filters, which may benefit from having the subject polymer composition infiltrated into adjacent tissue according to the present invention, include commercially available products. Examples of vena cava filters that can benefit from the incorporation of a fibrosis-inhibiting agent include, without limitation, the GÜNTHER TULIP Vena Cava FILTER and the GIANTURCO-ROEHM BIRD'S NEST Filter which are sold by Cook, Inc. (Bloomington, Ind.). C.R. Bard (Murray Hill, N.J.) sells the SIMON-NITINOL FILTER and RECOVERY Filter. Cordis Endovascular which is a subsidiary of Cordis Corporation (Miami Lakes, Fla.) sells the TRAPEASE Permanent Vena Cava Filter. B. Braun Medical Inc. (Bethlehem, Pa.) sells the VENA TECH LP Vena Cava Filter and VENA TECH—LGM Vena Cava Filter. Boston Scientific Corporation (Natick, Mass.) sells the Over-the-Wire GREENFIELD Vena Cava Filter.

In one aspect, the present invention provides inferior vena cava filter devices having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with inferior vena cava filters have been described above. These polymer compositions may comprise one or more fibrosis-inhibiting agents such that the overgrowth of granulation tissue is inhibited or reduced and/or one or more anti-infective agents such that infection is prevented or inhibited.

Polymeric compositions may be infiltrated around implanted inferior vena cava filter devices by applying the composition directly and/or indirectly into and/or onto (a) tissue adjacent to the inferior vena cava filter device; (b) the vicinity of the inferior vena cava filter device-tissue interface; (c) the region around the inferior vena cava filter device; and (d) tissue surrounding the inferior vena cava filter device. Methods for infiltrating the subject polymer compositions into tissue adjacent to an inferior vena cava filter device include delivering the polymer composition: (a) to the inferior vena cava filter device surface (e.g., as an injectable, paste, gel or mesh) during the implantation procedure; (b) to the surface of the tissue (e.g., as an injectable, paste, gel, in situ forming gel or mesh) immediately prior to, or during, implantation of the inferior vena cava filter device; (c) to the surface of the inferior vena cava filter device and/or the tissue surrounding the implanted inferior vena cava filter device (e.g., as an injectable, paste, gel, in situ forming gel or mesh) immediately after the implantation of the inferior vena cava filter device; (d) by topical application of the composition into the anatomical space where the inferior vena cava filter device may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the inferior vena cava filter device as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device.

According to one aspect, any fibrosis-inhibiting and/or anti-infective agent described above may be utilized in the practice of the present invention. In one aspect of the invention, the subject polymer compositions infiltrated into tissue adjacent to vena cava filters (e.g., inferior vena cava filters) may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As inferior vena cava filter devices are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10^{'7} to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

Gastrointestinal Devices

In one aspect, the subject polymer compositions may be infiltrated into tissue adjacent to a gastrointestinal (GI) device. The subject polymer compositions may contain a therapeutic agent (e.g., an anti-scarring and/or anti-infective agent). There are many gastrointestinal tube devices that are used for feeding applications and for drainage applications. The functioning of these tubes can be compromised if there is an excessive fibroproliferative response to these devices or an infection at the site of the device. Infiltration of a polymer composition containing a fibrosis-inhibiting and/or anti-infective agent into tissue adjacent to the device can modulate this fibroproliferative response (e.g., to prevent stenosis and/or obstruction of the device) thereby maintaining performance of the device and/or may prevent or inhibit infection at the site of the device.

A variety of GI tubes for drainage or feeding can be used in the present invention. These devices may include, without limitation, GI tubes for drainage or feeding, portosystemic shunts, shunts for ascites, nasogastric or nasoenteral tubes, gastrostomy or percutaneous feeding tubes, jejunostomy endoscopic tubes, colostomy devices, drainage tubes, biliary T-tubes, biopsy forceps, biliary stone removal devices, endoscopic retrograde cholangiopancretography (ERCP) devices, dilation balloons, enteral feeding devices, stents, low profile devices, virtual colonoscopy (VC) devices, capsule endoscopes, and retrieval devices.

GI devices may be composed of synthetic materials, including, without limitation, stainless steel, metals, nitinol, glass, resins or polymers.

In one aspect, the GI device may be an instrument used to examine or provide access to the interior of the gastrointestinal tract. This may include optical imaging in the form of still imaging or videoing for diagnosing purposes. Procedures that use these devices include, without limitation, enteroscopy, colonoscopy or esophagogastroduodenoscopy, where an endoscope enters the esophagus or anal canal to assess portions of the GI tract. For example, the GI device may be an endoscope having a tubular shaft for receiving a viewing lens and a treatment instrument. See, e.g., U.S. Pat. No. 5,421,323. The GI device may be a multi-lumen endoscopic catheter that may be inserted through an endoscope for the practice of endoscopic retrograde cholangiopancreatography, whereby the first lumen has a wire threaded through it, the second lumen provides a conduit to infuse a radio-opaque contrast medium to identify obstructions, and the third lumen provides a conduit to dilate a balloon. See, e.g., U.S. Pat. Nos. 5,788,681 and 5,843,028. The GI device may be a video endoscope system composed of a swallowable capsule, a transmitter and a reception system. See, e.g., U.S. Pat. No. 5,604,531. The GI device may be an endoscope composed of an encapsulated ultrasonic transducer capsule having a self-contained electromechanical sector scanner, which may be used for transesophageal echocardiography. See, e.g., U.S. Pat. Nos. 4,977,898 and 4,834,102. The GI device may be a sterilizable endoscope having an image sensor mounted on a cylindrical capsule and a separable disposable channel. See, e.g., U.S. Pat. No. 5,643,175. The GI device may be a body canal intrusion instrument that may be composed of a bidirectional surface friction for engaging tissue during navigation to decrease the risk of puncture and time associated with the insertion of catheters, guidewires and endoscopes through body cavities and canals. See, e.g., U.S. Pat. No. 6,589,213. The GI device may be a colonic access device composed of flexible tubing with a tether for releasing from a colonoscope, which may be placed in the colon for up to several days to monitor and treat colorectal diseases. See, e.g., U.S. Pat. No. 6,149,581. The GI device may be adapted for the bile or pancreatic duct by being composed of a mother endoscope that is inserted into the duodenum and a daughter endoscope that is inserted via papilla through a forceps channel. See, e.g., U.S. Pat. No. 4,979,496.

In another aspect, the GI device may be used as a conduit for long-term tube feeding. These GI devices may include, without limitation, percutaneous feeding tubes, enteral feeding devices/catheters, gastrostomy feeding tubes, low profile devices, and nasogastric tubes. These long-term feeding tubes may be advanced through the GI tract via nasal canal or through the abdominal wall via a gastrostomy. For example, the GI device may be an enteral feeding catheter adapted to serve as a conduit for passage of sustenance through an abdominal wall into the body and having a retainer and retractable locking means. See, e.g., U.S. Pat. No. 4,826,481. The GI device may be an enteral feeding tube having a catheter that allows for easy insertion and removal by having a slim, tapered guide tube and a balloon bolster. See, e.g., U.S. Pat. No. 6,582,395. The GI device may be an enteral feeding device for administering fluids into the stomach, which is composed of a female connector, flexible feeding tube, fluid discharge tube, and probe, which are connected to the male end of the guide wire. See, e.g., U.S. Pat. No. 5,242,429. The GI device may be a hollow, cylindrical elongated body with a spring-biased valve, which is maintained through a surgical opening in the stomach wall by an extended concentric flange that facilitates fixation. See, e.g., U.S. Pat. No. 4,344,435. The GI device may be a nasogastric tube having openings along its distal end with a coupled introducer flexible sheath extending longitudinally along the tube. See, e.g., U.S. Pat. No. 5,334,167. Other GI devices used as feeding tubes or related devices are described in, e.g., U.S. Pat. Nos. 6,582,395; 5,989,225; 5,720,734; 5,716,347; 5,503,629; 5,342,321; 4,861,334; 4,758,219 and 4,057,065.

In another aspect, the GI device may be used for irrigation or aspiration of the GI tract. These GI devices may be used, for example, to remove ingested poisons or blood, to treat absorption-related conditions, to decompress the stomach, pre-operatively to ensure portions of the GI tract is empty, post-operatively to remove gas, and to treat diseases such as bowel obstructions or paralytic ileus. For example, the GI tube may be elongated and configured to be inserted in the GI tract having a slidable treatment device for controlling bleeding and a fluid reservoir coupled to the tube. See, e.g., U.S. Pat. No. 5,947,926. The GI tube may be a nasogastric flexible tube with a curved or bent leading end to anatomically conform and facilitate advancement into the esophagus and stomach. See, e.g., U.S. Pat. No. 5,690,620. The GI tube may be a nasogastric elongated tube fixedly bent to extend from the nostril without affixation to avoid pressure necrosis in the nose due to force exertion. See, e.g., U.S. Pat. No. 4,363,323. The GI device may be composed of aspirating, feeding and inflation lumens, which is surgically inserted through the abdominal and gastric wall. See, e.g., U.S. Pat. No. 4,543,089. The GI device may be composed of drain tube and irrigating tube with a cuffed fluid sealing that is used for unidirectional irrigation of the bowels. See, e.g., U.S. Pat. No. 4,637,814. The GI device may be an open-ended, thin-walled, balloon-like tube shaped to extend through at least part of an alimentary canal for the purpose of passing digested food solids and thereby treating absorption-related diseases. See, e.g., U.S. Pat. Nos. 4,315,509 and 4,134,405.

In another aspect, the GI device may be a colostomy device. For example, the colostomy device may be an artificial anus composed of a hollow tubular support with a cylindrical body having a pair of radially-extending flanges to engage the member See, e.g., U.S. Pat. No. 4,781,176. The colostomy device may be composed of internal and external balloons connected by a tube and an annular supporting plate for attachment to the stoma or rectum. See, e.g., U.S. Pat. No. 5,569,216.

In another aspect, the GI device may be a mechanical hemostatic device used to control GI bleeding. Hemostatic devices, which are used to constrict blood flow, may include, without limitation, clamps, clips, staples and sutures. For example, the hemostatic device may be a compression clip composed of an anchor and stem having a transverse hole and a bolster which may be fixed or movable along the stem. See, e.g., U.S. Pat. No. 6,387,114. The hemostatic device may be an endoscopic clip composed of deformable material and a tissue-penetrating pair of hollow jaws. See, e.g., U.S. Pat. No. 5,989,268.

In another aspect, the GI device may be a means to clear blocked GI tracts. For example, the GI device may be a dilation catheter composed of a shroud tube having a strain relief tube extending from within which is used to alter the configuration of a dilation balloon. See, e.g., U.S. Pat. No. 6,537,247.

In another aspect, the GI device may function to deliver drug to the GI tract. For example, the GI device may be orally administered and composed of a two-chambered water-permeable body, in which one chamber has an orifice for expelling a liquid drug when under pressure, and the second chamber contains an electric circuit that generates a gas which compresses the first chamber to expel the drug. See, e.g., U.S. Pat. No. 5,925,030. The GI device may be a collapsible, ellipsoidal gastric anchor with a tether and a long, narrow intestinal payload module, which contains slow release medicaments, bound enzymes or nonpathogenic microorganisms. See, e.g., U.S. Pat. No. 4,878,905. The GI device may be an ingestible device for delivering a substance to a chosen site within the GI tract, which includes a receiver of electromagnetic radiation for powering an openable part of the device for inserting or dispensing the substance. See, e.g., U.S. Pat. No. 6,632,216.

In another aspect, the GI device may be a shunting device used to provide communication between two bodily systems. Shunting devices may be used to treat abnormal conditions, such as bypassing occlusions in a body passageway or transferring unwanted accumulation of fluids from a body cavity to a site where it can be processed by the body. For example, a shunting device may be used to displace peritoneal cavity fluid into the systemic venous circulation as a treatment for ascites. Shunting devices may include, without limitation, portosystemic shunts and peritoneovenous shunts. For example, the shunt may be an implantable pump composed of a cylindrical chamber and port with pumping means for aspirating fluid and expelling fluids. See, e.g., U.S. Pat. No. 4,725,207. The shunt may be an implantable peritoneovenous shunt system composed of a double-chambered ascites collection device, a pump (e.g., magnetically driven or compression driven), and an anti-reflux catheter, that are all connected by flexible tubing. See, e.g., U.S. Pat. No. 4,657,530 and 4,610,658. The shunt may be composed of a peritoneal tube connected to a hollow plastic implanted valve assembly that passes fluid when under pressure to a venous tube. See, e.g., U.S. Pat. No. 5,520,632. The shunt may be a collapsible, shape-memory metal fabric with a plurality of woven metal strands having a central passageway for fluid and delivered in a collapsed state through a body channel to create a portosystemic shunt. See, e.g., U.S. Pat. No. 6,468,303. The GI device may be a laparoscopic tunneling dissector composed of an inflatable balloon and a hollow blunt tipped obturator which is used to tunnel through tissue to provide an anatomic working space for laparoscopic procedures. See, e.g., U.S. Pat. Nos. 5,836,961 and 5,817,123.

GI devices, which may benefit from having the subject polymer composition infiltrated into adjacent tissue according to the present invention, include commercially available products.

In one aspect, GI devices that are used for feeding purposes may include a variety of devices. For example, gastrostomy tubes such as the DURA-G Polyurethane Gastrostomy Tubes and MAGNA-PORT Gastrostomy Tubes are sold by Ross Products (Columbus, Ohio), a division of Abbott Laboratories. Moss Tubes, Inc. (West Sand Lake, N.Y.) sells the MOSS G-Tube Percutaneous Endoscopic Gastrostomy Kits. Other enteral feeding tubes include, for example, EASY-FEED Enteral Feeding Sets which are sold by Ross Products (Columbus, Ohio), a division of Abbott Laboratories. COMPAT Enteral Delivery Systems are sold by Novartis AG (Basel, Switzerland). CORFLO Feeding Tubes are sold by VIASYS Healthcare Medsystems Division (Wheeling, Ill.). ENDOVIVE Enteral Feeding Systems are sold by Boston Scientific Corporation. Nasogastric tubes, such as the Mark IV Nasal (SIL) Tubes are sold by Moss Tubes, Inc. (West Sand Lake, N.Y.). Bard Medical Division (Covington, Ga.) of C.R. Bard, Inc. and Andersen Products Limited (England, United Kingdom) also sells a variety of Nasogastric Feeding Tubes. Low profile devices, such as the Low-Profile Replacement Gastrostomy Devices and the Bard Button Replacement Gastrostomy Devices are sold by Bard Endoscopic Technologies (Billerica, Mass.), a division of C.R. Bard, Inc.

In another aspect, GI devices may include gastrointestinal tubes for irrigation or aspiration, such as the LAVACUATOR Gastro Intestinal Tubes and VENTROL Levine Tubes, which are sold by Nellcor Puritan Bennett Inc. (Pleasanton, Calif.).

In another aspect, GI devices may include those used as portosystemic shunts or other shunting devices, such as the VIATORR TIPS Endoprostheses that are sold by W.L. Gore & Associates, Inc. (Newark, Del.). Denver Ascites Shunts are sold by Denver Biomedical, Inc. (Golden, Colo.). LEVEEN Shunts are sold by Becton, Dickinson and Company (Franklin Lakes, N.J.).

In another aspect, GI devices may include colostomy devices, such as ASSURA Pouches and COLOPLAST Pouches, which are sold by Coloplast Corporation (Marietta, Ga.). ESTEEM SYNERGY Standard Closed-End Pouches and SUR-FIT NATURA Closed-End Pouches are sold by ConvaTec (Princeton, N.J.), a Bristol-Myers Squibb Company. Cymed Ostomy Company (Berkeley, Calif.) sells the MICROSKIN Colostomy Pouching Systems. KARAYA 5 One-Piece Pouching Systems, CONTOUR I One-Piece Ostomy Pouching Systems, and CENTERPOINTLOCK (CPL) Two-Piece Pouching Systems are sold by Hollister Inc. (Libertyville, Ill.). Bard Medical Division (Covington, Ga.) of C.R. Bard, Inc. also sells a variety of Colostomy Pouches.

In another aspect, GI devices may include dilatation catheters, such as the ELIMINATOR Multi-Stage Balloon Dilators, which are sold by Bard Endoscopic Technologies (Billerica, Mass.), a division of C.R. Bard, Inc. CRE Fixed Wire and Wireguided Balloon Dilators are sold by Boston Scientific Corporation (Natick, Mass.).

In one aspect, the present invention provides GI devices having the subject polymer compositions infiltrated into adjacent tissue, where the subject polymer compositions may include a therapeutic agent (e.g.; an anti-scarring and/or anti-infective agent). Numerous polymeric and non-polymeric delivery systems for use in connection with GI devices have been described above. These polymer compositions may comprise one or more fibrosis-inhibiting agents such that the overgrowth of granulation tissue is inhibited or reduced and/or one or more anti-infective agents such that infection is prevented or inhibited.

Polymeric compositions may be infiltrated around implanted GI devices by applying the composition directly and/or indirectly into and/or onto (a) tissue adjacent to the GI device; (b) the vicinity of the GI device-tissue interface; (c) the region around the GI device; and (d) tissue surrounding the GI device. Methods for infiltrating the subject polymer compositions into tissue adjacent to a GI device include delivering the polymer composition: (a) to the GI device surface (e.g., as an injectable, paste, gel or mesh) during the implantation procedure; (b) to the surface of the tissue (e.g., as an injectable, paste, gel, in situ forming gel or mesh) immediately prior to, or during, implantation of the GI device; (c) to the surface of the GI device and/or the tissue surrounding the implanted GI device (e.g., as an injectable, paste, gel, in situ forming gel or mesh) immediately after the implantation of the GI device; (d) by topical application of the composition into the anatomical space where the GI device may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the GI device as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device.

According to one aspect, any anti-scarring and/or anti-infective agent described above may be utilized in the practice of the present invention. In one aspect of the invention, the subject polymer compositions infiltrated into tissue adjacent to GI devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As GI devices are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

Central Venous Catheters

In one aspect, the subject polymer compositions may be infiltrated into tissue adjacent to a central venous catheter (CVC) device. The subject polymer compositions may contain a therapeutic agent (e.g., an anti-scarring and/or anti-infective agent). For the purposes of this invention, the term “Central Venous Catheters” should be understood to include any catheter or line that is used to deliver fluids to the large (central) veins of the body (e.g., jugular, pulmonary, femoral, iliac, inferior vena cava, superior vena cava, axillary etc.). CVC devices are generally hollow, tubular cannulae that are inserted into body passageways to permit injection or withdrawal of bodily fluids. CVCs may be inserted into a large vein, such as the superior vena cava, with a portion of the catheter disposed within the body and a connection port which extends out of the body for access to the circulatory system. CVCs may be used to administer drugs (e.g., chemotherapy or antibiotic therapy) or intravenous feeding, pressure monitoring or periodic blood sampling.

CVCs may be designed with or without a cuff or flange. Cuffs are used to prevent the catheter from slipping or becoming infected. CVCs may have one lumen or multiple lumens and range in many sizes to adapt to the required needs. They may be composed of synthetic materials, including, but not limited to, polyurethane, polyethylene, silicone, copolymers and other polymeric compositions.

CVCs are typically left in the body for a long period of time and thus, may develop infection or inflammation in response to the catheter. CVC access lumens may be blocked by clotted blood or thrombus formation. Some CVCs may also be available with coatings and treated surfaces to minimize the risk of infection and/or inflammation. Infiltration of a polymer composition containing a fibrosis-inhibiting and/or anti-infective agent into tissue adjacent to the device can modulate an excessive fibroproliferative response to the device, which may prevent stenosis and/or obstruction of the device, and/or may prevent or inhibit infection at the site of the device.

In one aspect, the CVC may be designed for specialized access to the circulatory system for specific conditions/purposes. For example, the CVC may be especially made for hemodialysis use by being elongated with a needle-like, dual lumen that may be used as a conduit for administering drugs or additives into the body through an AV access fistula or graft. See, e.g., U.S. Pat. No. 5,876,366. The CVC may be composed of an indwelling cannula adapted for placement within the superior vena cava having an exit port at the distal end whereby fluid medicament may be delivered to essentially the area of subcutaneous tissue surrounding the cannula. See, e.g., U.S. Pat. No. 5,817,072.

In another aspect, the CVC may be designed to provide multiple conduits for accessing the circulatory system. For example, the CVC may be an elongated, integral flexible catheter tube with a plurality of independent lumens that may be adapted for attachment to a separate fluid conveying device whereby fluids may be separately infused into the vein without becoming mixed, and blood may be withdrawn and venous pressure monitored simultaneously with fluid infusion. See, e.g., U.S. Pat. No. 4,072,146. The CVC may be a multi-lumen catheter composed of a central flexible lumen with a formed fluid passageway and a plurality of collapsible lumens mounted around the periphery of the central lumen also having formed fluid passageways therein. See, e.g., U.S. Pat. No. 4,406,656.

In another aspect, the CVC may have a means for preventing infection as a result of long-term use. For example, the CVC may be composed of polyurethane with a thin hydrophilic layer on the surface loaded with an antibiotic of the ramoplanin group to inhibit bacterial colonization on the catheter after insertion. See, e.g., U.S. Pat. No.5,752,941. The CVC may be composed of a polymeric material that has an outer surface embedded by atoms of an antimicrobial metal (e.g., silver) that extend in a subsurface stratum to form a nonleaching surface treatment. See, e.g., U.S. Pat. No. 5,520,664.

In another aspect, the CVC may be used with an apparatus that provides a means of controlling the injection or withdrawal of bodily fluids through the CVC. For example, the CVC apparatus may be composed of a syringe body with two barrels that have two separate fluid conduits with independent plungers and a valve body. See, e.g., U.S. Pat. No. 5,411,485. The CVC apparatus may be composed of an upper and lower molded sheets and a plurality of syringe channels and barrels that are individually operated by syringe plungers. See, e.g., U.S. Pat. No. 5,417,667. The CVC apparatus may be an integrally molded base sheet which forms opposed slide valve walls that have a plurality of syringes mounted for fluid communication with the inlet ports. See, e.g., U.S. Pat. No. 5,454,792. The CVC apparatus may be composed with access apparatus to provide easier accessibility by being composed of a connector that is in bidirectional fluid communication between a manifold and a CVC. See, e.g., U.S. Pat. No. 5,308,322. The CVC apparatus may be a valve assembly that is provided for the distal end of a CVC for controlling fluid passage from the catheter to the blood flow passage in which it is inserted. See, e.g., U.S. Pat. No. 5,030,210.

Other examples of central venous catheters include total parenteral nutrition catheters, peripherally inserted central venous catheters, flow-directed balloon-tipped pulmonary artery catheters, long-term central venous access catheters (such as Hickman lines and Broviac catheters). Representative examples of such catheters are described in U.S. Pat. Nos. 3,995,623, 4,072,146 4,096,860, 4,099,528, 4,134,402, 4,180,068, 4,385,631, 4,406,656, 4,568,329, 4,960,409, 5,176,661, 5,916,208.

CVCs, which may benefit from having the subject polymer composition infiltrated into adjacent tissue according to the present invention, include commercially available products. For example, Bard Access Systems (Salt Lake City, Utah) which is a division of C.R. Bard sells the HICKMAN, BROVIAC and LEONARD Central Venous Catheters which are available with SureCuff tissue in-growth cuff and the VitaCuff Antimicrobial Cuff. Edward Lifesciences (Irvine, Calif.) sells the VANTEX Catheter as well as the PRESEP CENTRAL VENOUS OXIMETRY Catheter. Cook Critical Care (Bloomington, Ind.) sells the SPECTRUM Antibiotic Impregnated Catheters as well as other CVC sets and trays. Arrow International (Reading, Pa.) sells the ARROWGARD BLUE Catheters that have single or multiple lumens.

A variety of central venous catheters are available for use in hemodialysis including, but not restricted to, catheters which are totally implanted such as the Lifesite (Vasca Inc., Tewksbury, Mass.) and the Dialock (Biolink Corp., Middleboro, Mass.). Central venous catheters are prone to infection and aspects of the present invention for prevention or inhibition of infection are described above.

In one aspect, the present invention provides CVC devices having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with CVC devices have been described above. These polymer compositions may comprise one or more fibrosis-inhibiting agents and/or one or more anti-infective agents such that the overgrowth of granulation tissue is inhibited or reduced and/or infection at the site of the CVC device is inhibited or prevented.

Polymeric compositions may be infiltrated around implanted CVC devices by applying the composition directly and/or indirectly into and/or onto (a) tissue adjacent to the CVC device; (b) the vicinity of the CVC device-tissue interface; (c) the region around the CVC device; and (d) tissue surrounding the CVC device. Methods for infiltrating the subject polymer compositions into tissue adjacent to a CVC device include delivering the polymer composition: (a) to the CVC device surface (e.g., as an injectable, paste, gel or mesh) during the implantation procedure; (b) to the surface of the tissue (e.g., as an injectable, paste, gel, in situ forming gel or mesh) immediately prior to, or during, implantation of the CVC device; (c) to the surface of the CVC device and/or the tissue surrounding the implanted CVC device (e.g., as an injectable, paste, gel, in situ forming gel or mesh) immediately after the implantation of the CVC device; (d) by topical application of the composition into the anatomical space where the CVC device may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the CVC device as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device.

In some aspects, the subject polymer compositions may infiltrated into tissue adjacent to: (a) the exterior surface of the intravascular portion of the CVC device and/or the segment of the CVC device that traverses the skin; (b) exterior surface of the intravascular portion of the CVC device and/or the segment of the CVC device that traverses the skin, where the interior and/or exterior of the CVC device is coated with a polymer composition comprising a therapeutic agent (e.g., an anti-infective agent); (c) the surface of, a subcutaneous “cuff” around the CVC device; (d) other surfaces of the CVC device; and (e) any combination of the aforementioned.

According to one aspect, any anti-scarring and/or anti-infective agent described above may be utilized in the practice of the present invention. In one aspect of the invention, the subject polymer compositions infiltrated into tissue adjacent to CVC devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As CVC devices are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

Ventricular Assist Devices

In one aspect, the subject polymer compositions may be infiltrated into tissue adjacent to a ventricular assist device (VAD). The subject polymer compositions may contain a therapeutic agent (e.g., an anti-scarring and/or anti-infective agent).

VADs are intended to assist the native heart in pumping blood throughout the body. Examples of VADs and other related devices include, without limitation, left ventricular assist devices, right ventricular assist devices, biventricular assist devices, cardiac assist devices, mechanical assist devices, artificial cardiac assist devices, implantable heart assist systems, implantable ventricular assist devices, heart assist pumps and intra-ventricular cardiac assist devices.

VADs are used to treat heart failure where the heart is incapable of pumping blood throughout the body at the rate needed to maintain adequate blood flow. Heart failure includes, without limitation, acute myocardial infarction, cardiomyopathy, cardiac valvular dysfunction, extensive cardiac surgery and uncontrolled cardiac arrhythmias. VADs assist the failing heart by increasing its pumping ability and allowing the heart to rest to recover its normal pumping function. In general, VADs are typically composed of a blood pump that is attached between the ventricle and aorta, cannulae that connect the pump to the heart, and a drive console that powers and controls the device. The most common VAD that exists is the left VAD because the left ventricle of the heart becomes diseased more often than the right ventricle; however, VADs may be used to pump blood from the left ventricle, right ventricle or both ventricles. VADs may be categorized by the pumping drives, which may function as either pulsatile (e.g., intra-aortic balloon pumps) or continuous, (e.g., reciprocating piston-type pumps or rotary pumps (centrifugal or axial impellers)).

VADs, however, may have medical complications associated with the implantation or prolonged use, such as, infections, septic emboli, hemorrhaging, inflammation as a reaction to tissue damage, and thrombosis induced by coagulation or blood stasis. These complications may obstruct the utility of the VAD and may lead to life threatening events. Infiltration of a polymer composition containing an anti-scarring and/or anti-infective agent into tissue adjacent to a VAD may prevent stenosis and/or obstruction of the device and/or may prevent or inhibit infection at the site of the device.

In one aspect, the VAD may be a pulsatile pump. These devices may have flexible sacks or diaphragms which are compressed and released to provide pulsatile pumping action. One type of pulsatile pump is the intra-aortic balloon pumps (IABP) which is a pulsatile sack device that may be implemented using minimally invasive procedures and are most functional when the left ventricle is able to eject blood to maintain a systemic arterial pressure. For example, the VAD may be an IABP that is a temporary, removable support within the aortic arch that descends through the aorta which has both a depressurized and pressurized position which is maintained by a pumping and blocking balloon. See, e.g., U.S. Pat. No. 6,228,018. The VAD may be an IABP catheter and a pumping chamber having both a large and small diameter portions that are separated by a flexible diaphragm/membrane. See, e.g., U.S. Pat. No. 5,928,132. The VAD may be a pulsatile pump composed of a cannula with an outer sheath and lumen, intake and outlet valves, fluid reservoir, and hydraulic pump that produces a pulsatile pumping action of blood through the cannula. See, e.g., U.S. Pat. No. 6,007,479.

In another aspect, the VAD may be a continuous pump providing mostly steady flow of blood which may include an imperceptible pulsatile component. Continuous pumps may include reciprocating piston-type pumps, such as pneumatically powered devices or magnetically operated devices, and rotary pumps, such as centrifugal or axial impellors. For example, the VAD may be an implantable apparatus with a stator member and a magnetically suspended rotor member that act as a centrifugal pump where an impeller draws blood from the left ventricle and delivers it to the aorta thereby reducing the left ventricle pressure. See, e.g., U.S. Pat. No. 5,928,131. The VAD may be composed of an implantable reciprocating piston for driving an implanted blood-pumping mechanism which is controlled by external electromagnets. See, e.g., U.S. Pat. No. 5,089,017.

In another aspect, the VAD may be a device for assisting the pumping capacity of one of either the left or right ventricle. For example, the VAD may be composed of a housing apparatus with a pair of chambers with an inlet and outlet port, at least one ventricular outflow conduit, and an actuator that contracts one of the chambers while expanding the other to provide a positive displacement pump. See, e.g., U.S. Pat. No. 6,264,601. The VAD may be composed of a pump, a chamber above the pump, and a tube that connects the pump and chamber using liquid and gas as a means for communication. See, e.g., U.S. Pat. No. 6,146,325.

In another aspect, the VAD may be a device designed specifically for the left ventricle. For example, the VAD may be a blood pump adapted to be joined in flow communication between the left ventricle and the aorta using an inlet flow pressure sensor and a controller that may adjust speed of pump based on sensor feedback. See, e.g., U.S. Pat. No. 6,623,420. The VAD may be composed of a bag adapted to expand by being filled with blood and able to contract to expel the blood, and the means for varying the resistance of the bag by using gaseous substance through a duct to a containing casing. See, e.g., U.S. Pat. No. 6,569,079. The VAD may be a pump system composed of a deformable sac with inlet and outlet means and a pair of plates on opposite sides of the sac to deform the sac. See, e.g., U.S. Pat. No. 5,599,173.

In another aspect, the VAD may be a device designed as a biventricular assist device. For example, the VAD may be a biventricular assist device composed of a self-supporting cup having an annular diaphragm that forms a fluid chamber around the heart cavity whereby it may have a pressure inlet/port that communicates with the fluid chamber to regulate positive and negative pressures. See, e.g., U.S. Pat. No. 5,908,378; 5,749,839 and 5,738,627.

In another aspect, the VAD may be an implanted system used to supplement the pumping of blood circulation from a location outside the heart. For example, the VAD may be an extracardiac pumping system composed of an inflow and outflow conduit fluidly coupled to the pump (e.g., pulsatile or rotary pump) and a control circuit to synchronously actuate the pump. See, e.g., U.S. Pat. Nos. 6,610,004; 6,428,464 and 6,200,260.

In another aspect, the VAD-related devices may be a used in conjunction with VADs or as stand alone to treat congestive heart failure victims. For example, a VAD-related device may be a reinforcement device composed of a jacket that is applied to the heart to constrain cardiac expansion to a predetermined limit. See, e.g., U.S. Pat. Nos. 6,582,355; 6,567,699; 6,241,654 and 6,169,922.

Representative examples of VADs, which may benefit from having the subject polymer composition infiltrated into adjacent tissue according to the present invention, include commercially available products. For example, Thoratec Corporation (Pleasanton, Calif.) sells the HEARTMATE Left Ventricular Assist Systems. WorldHeart Corporation (ON, Canada) sells the WORLDHEART NOVACOR Left Ventricular Assist System. Arrow International (Reading, Pa.) sells the LIONHEART Left Ventricular Assist System.

In one aspect, the present invention provides LVAD having the subject polymer composition infiltrated into adjacent tissue, where the polymer composition may comprise an anti-scarring and/or anti-infective agent. Numerous polymeric and non-polymeric delivery systems for use in connection with VADs have been described above. These polymer compositions may comprise one or more fibrosis-inhibiting agents and/or one or more anti-infective agents such that the overgrowth of granulation tissue is inhibited or reduced and/or infection at the site of the VAD is inhibited or prevented.

Polymeric compositions may be infiltrated around implanted VADs by applying the composition directly and/or indirectly into and/or onto (a) tissue adjacent to the VAD; (b) the vicinity of the VAD-tissue interface; (c) the region around the VAD; and (d) tissue surrounding the VAD. Methods for infiltrating the subject polymer compositions into tissue adjacent to a VAD include delivering the polymer composition: (a) to the VAD surface (e.g., as an injectable, paste, gel or mesh) during the implantation procedure; (b) to the surface of the tissue (e.g., as an injectable, paste, gel, in situ forming gel or mesh) immediately prior to, or during, implantation of the VAD; (c) to the surface of the VAD and/or the tissue surrounding the implanted VAD (e.g., as an injectable, paste, gel, in situ forming gel or mesh) immediately after the implantation of the VAD; (d) by topical application of the composition into the anatomical space where the VAD may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the VAD as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device.

According to the one aspect, any anti-scarring and/or anti-infective agent described above may be utilized in the practice of the present invention. In one aspect of the invention, the subject polymer compositions infiltrated into tissue adjacent to VADs (e.g., LVAD's) may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As VADs are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

Spinal Implants

In one aspect, the subject polymer compositions may be infiltrated into tissue adjacent to a spinal implant (e.g., a spinal prosthesis). The subject polymer compositions may contain a therapeutic agent (e.g., an anti-scarring and/or anti-infective agent). As used herein, the term “spinal prostheses” refers to devices that are located in, on, or near the spine and which enhance the ability of the spine to perform its function in the host. Spinal prostheses may be used to treat the vertebral column following degeneration or damage to the spine or a component or portion thereof. In healthy hosts, the vertebral column is composed of vertebral bone plates separated by intervertebral discs that form strong joints and absorb spinal compression. The intervertebral disc is comprised of an inner gel-like substance called the nucleus pulposus with surrounding tough fibrocartilagenous fibers called the annulus fibrosis. When damage occurs to the intervertebral disc, the host can develop spinal dysfunction, crippling pain, as well as long-term disability. Typically, damage to an intervertebral disc requires surgery which often results in the fusion of adjacent vertebral bone plates using various techniques and devices. Fusion of vertebral segments alleviates the pain by restricting vertebral motion at the damaged intervertebral disc. When only one vertebral segment is fused, the host will not have any noticeable motion limitations. However, when two or more segments are fused, the normal motion of the back may become limited and thus, pain relief may not resolve due to the additional stress that is induced across the remaining vertebral joints.

In one aspect, the damaged vertebral segment may be treated using a spinal prosthesis that induces fusion between the vertebral plates. This may be conducted when only one vertebral segment is damaged. In another aspect, the damaged vertebral segment may be treated using a spinal prosthesis that maintains vertebral movement within the vertebral joint. This may be conducted when damage to more than one vertebral segment occurs.

Examples of spinal prostheses include, without limitation, spinal discs and related devices including vertebral implants, vertebral disc prostheses, lumbar disc implants, cervical disc implants, intervertebral discs, implantable prostheses, spinal prostheses, artificial discs, prosthetic implants, prosthetic spinal discs, spinal disc endoprostheses, spinal implants, artificial spinal discs, intervertebral implants, implantable spinal grafts, implantable bone grafts, artificial lumbar discs, spinal nucleus implants, and intervertebral disc spacers. Also included within the term spinal prostheses are fusion cages and related devices including fusion baskets, fusion cage apparatus, interbody cages, interbody implants, fusion devices, fusion cage anchoring devices, bone fixation apparatus, bone fixation instrumentation, bone fixation devices, fusion stabilization chamber, fusion cage anchoring plates, anchoring bone plates and bone screws.

A spinal prosthesis according to the present invention may be composed of a single material or a variety of materials including, without limitation, allograft bone material (see, e.g., U.S. Pat. No. 6,143,033), metals (see, e.g., U.S. Pat. No. 4,955,908), and/or synthetic materials (see, e.g., U.S. Pat. No. 6,264,695, 6,419,706, 5,824,093 and 4,911,718). The prosthesis must be biocompatible. It may consist of biodegradable or non-biodegradable components depending on the intended function of the device. See, e.g., U.S. Pat. No. 4,772,287. The spinal prosthesis may be biologically inert and serve as a mechanical means of stabilizing the vertebral column (see, e.g., U.S. Pat. Nos. 4,955,908 and 5,716,415) or it may be biologically active and serve to promote fusion with the adjacent vertebral bone plates (see, e.g., U.S. Pat. Nos. 5,489,308 and 6,520,993).

In one aspect, the prosthesis may be a fusion cage designed to promote vertebral fusion in order to limit movement between adjacent vertebrae. Fusion cages may be interbody devices that fit within the intervertebral space or they may encompass both the intervertebral space and the anterior region of the vertebral column. Fusion cages may have various shapes. For example, fusion cages may be have a rectangular shape or may be cylindrical in shape and may have a plurality of openings and helical threading. Fusion cages may have an outer body and a hollow cavity that may or may not be used to insert bone growth-promoting material for stimulating bone fusion. For example, the prosthesis may be an interbody fusion cage that has an externally threaded stem projecting from a domed outer end which is fixed using an assembly of a plate, a fastener and bone screws. See, e.g., U.S. Pat. No. 6,156,037. The prosthesis may be a fusion cage with a threaded outer surface adapted for promoting fusion with bone structures when a bone-growth-inducing substance is packed into the cage body. See, e.g., U.S. Pat. Nos. 4,961,740, 5,015,247, 4,878,915 and 4,501,269. The prosthesis may be a generally tubular shell with a helical thread projecting with a plurality of pillars with holes to facilitate bone ingrowth and mechanical anchoring. See, e.g., U.S. Pat. Nos. 6,071,310 and 5,489,308. Other U.S. patents that describe the threaded spinal implant include U.S. Pat. Nos. 5,263,953, 5,458,638 and 5,026,373.

In another aspect, the prosthesis may be a bone fixation device designed to promote vertebral fusion in order to limit movement between adjacent vertebrae. For example, bone dowels, rods, hooks, wires, wedges, plates, screws and other components may be used to fix the vertebral segments into place. The fixation device may fit within the intervertebral space or it may encompass both the intervertebral space and the anterior region of the vertebral column or it may only encompass the anterior region of the vertebral column. A bone fixation device may be used with a fusion cage to assist in stabilizing the device within the intervertebral area. For example, the prosthesis may be in the form of a solid annular body having a plurality of discrete bone-engaging teeth protruding on the superior and inferior surfaces and having a central opening that may be filled with a bone growth-promoting material. See, e.g., U.S. Pat. No. 6,520,993. The prosthesis may have a disk-like body with weld-like raised parts disposed on opposite surfaces to enhance lateral stability in situ. See, e.g., U.S. Pat. No. 4,917,704. The prosthesis may be composed of opposite end pieces that maintain the height of the intervertebral space with an integral central element that is smaller in diameter wherein osteogenic material is disposed within the annular pocket between the end pieces. See, e.g., U.S. Pat. No. 6,146,420. The prosthesis may be composed of first and second side surfaces extending parallel to each other with upper and lower surfaces that engage the adjacent vertebrae. See, e.g., U.S. Pat. No. 5,716,415. The prosthesis may be a fusion stabilization chamber composed of a hollow intervertebral spacer and an end portion with at least one hole for affixing into the surrounding bone. See, e.g., U.S. Pat. No. 6,066,175. The prosthesis may be composed of a metallic body tapering conically from the ventral to the dorsal end and having a plurality of fishplates extending from opposite sides with openings for bone screws. See, e.g., U.S. Pat. No. 4,955,908. The prosthesis may be composed of a pair of plates which may have protrusions for engaging the adjacent vertebrae and an alignment device disposed between the engaging plates for separating the plates to maintain them in lordotic alignment. See, e.g., U.S. Pat. No. 6,576,016. The prosthesis may be a plurality of implants that are inserted side by side into the disc space that promote bone fusion across an intervertebral space. See, e.g., U.S. Pat. No. 5,522,899. The prosthesis may be an anchoring device composed of an anchoring plate with a central portion configured for attachment to a vertebral implant (e.g., fusion cage) and the end portions adapted to fasten in a fixed manner to a bony segment of the vertebra. See, e.g., U.S. Pat. No. 6,306,170. The prosthesis may be a bone fixation apparatus composed of a bone plate and a fastener apparatus (e.g., bone screws). See, e.g., U.S. Pat. Nos. 6,342,055, 6,454,769, 6,602,257 and 6,620,163.

In another aspect, the prosthesis may be an alternative to spinal fusion. The prosthesis may be a disc designed to provide normal movement between vertebral bone plates. The disc may be intended to mimic the natural shock absorbent function of the natural disc. The disc may be composed of a center core and end elements that support the disc against the adjacent vertebra or it may be intended to replace only a portion of the natural intervertebral disc (e.g., nucleus pulposus). For example, the disc may be in the form of an elastomeric section sandwiched between two rigid plates. See, e.g., U.S. Pat. Nos. 6,162,252; 5,534,030, 5,017,437 and 5,031,437. The disc may be an elongated prosthetic disc nucleus composed of a hydrogel core and a constraining flexible jacket that allows the core to deform and reform. See, e.g., U.S. Pat. No. 5,824,093. The disc may be composed of a rigid superior and inferior concaval-convex elements and a nuclear body which is located between the concave surfaces to permit movement. See, e.g., U.S. Pat. No. 6,156,067. The disc may be a partial spinal prosthesis composed of a core made of an elastic material such as silicone polymer or an elastomer which is covered by a casing made of a rigid material which is in contact with the adjacent vertebrae. See, e.g., U.S. Pat. No. 6,419,706. The disc may replace only the nucleus pulposus tissue by using a spinal nucleus implant comprised of a swellable, biomimetic plastic with a hydrophobic and hydrophilic phase which can be expanded in situ to conform to the natural size and shape. See, e.g., U.S. Pat. No. 6,264,695. The disc may be composed of a central core formed from a biocompatible elastomer wrapped by multi-layered laminae made from elastomer and fibers. See, e.g., U.S. Pat. No. 4,911,718. The disc may be composed of a fluid-filled inner bladder with an outer layer of strong, inert fibers intermingled with a bioresorbable material which promotes tissue ingrowth. See, e.g., U.S. Pat. No. 4,772,287.

In another aspect, the spinal implant may be a device that reduces spine compression or reduces adhesions that may form as a result to spinal surgery and/or trauma. For example, the device may be a protection device composed of a shield to fit onto at least one lamina on the posterior surface to prevent postoperative formation of adhesions to the spinal dura. See, e.g., U.S. Pat. Nos. 5,437,672 and 5,868,745 and U.S. patent application No. 2003/0078588. The device may be a prosthesis having a patch flange and a suture flange extending circumferentially around the patch such that the tissue underlying the patch is shielded and effectively nonadhesive to scar growth. See, e.g., U.S. Pat. No. 5,634,944. The device may be a protective intervening barrier composed of a biocompatible shield which is used following intraspinal or vertebral surgery to prevent postoperative adhesions from binding onto the spinal nerves. See, e.g., U.S. Pat. No. 4,013,078. The device may be used for neuro decompression while reducing fibroplasia proximate to the nerve tissue by having a surface topography texturized with outwardly-extending microstructures. See, e.g., U.S. Pat. No. 6,106,558 and U.S. patent application No. 2003/0078673.

Spinal prostheses and other spinal implants, which may benefit from having the subject polymer composition infiltrated into adjacent tissue according to the present invention, include commercially available products. Medtronic Sofamor Danek (Memphis, Tenn.) sells the fusion cage product INTERFIX Threaded Fusion Device. Centerpulse Spine-Tech (Minneapolis, Minn.) sells the BAK/C Cervical Interbody Fusion System fusion cage product and the CERVI-LOK Cervical Fixation System fixation device. Spinal Concepts (Austin, Tex.) sells the SC-ACUFIX Anterior Cervical Plate System. DePuy Spine, Inc. (Raynham, Mass.) sells the spinal discs, ACROFLEX TDR prostheses and the CHARITÉ Artificial Disc. Synthes-Stratec (Switzerland) sells the PRODISC system, including the PRODISC Cervical-C IDE disc replacement. Raymedica, Inc. (Minneapolis, Minn.) sells the PDN (PROSTHETIC DISC NUCLEUS).

In one aspect, the present invention provides spinal implants having the subject polymer compositions infiltrated into adjacent tissue, where the subject polymer compositions may include a therapeutic agent (e.g., an anti-scarring and/or anti-infective agent). Numerous polymeric and non-polymeric carrier systems that can be used in conjunction with spinal implants have been described above. Infiltration of the subject polymer compositions comprising a fibrosis-inhibiting agent and/or anti-infective agent into tissue adjacent to a spinal implant can minimize fibrosis (or scarring) in the vicinity of the implant and/or may reduce or prevent the formation of adhesions between the implant and the surrounding tissue and/or may inhibit or prevent infection in the vicinity of the implant.

In one aspect, the present invention provides spinal implants having the subject polymer compositions infiltrated into adjacent tissue, where the subject polymer compositions may include a therapeutic agent (e.g., an anti-scarring and/or anti-infective agent) to inhibit scarring and adhesion between the device and the surrounding bone and/or inhibit or prevent infection at the site of the implant.

Polymeric compositions may be infiltrated around implanted spinal implants by applying the composition directly and/or indirectly into and/or onto (a) tissue adjacent to the spinal implant; (b) the vicinity of the spinal implant-tissue interface; (c) the region around the spinal implant; and (d) tissue surrounding the spinal implant. Methods for infiltrating the subject polymer compositions into tissue adjacent to a spinal implant include delivering the polymer composition: (a) to the spinal implant surface (e.g., as an injectable, paste, gel or mesh) during the implantation procedure; (b) to the surface of the tissue (e.g., as an injectable, paste, gel, in situ forming gel or mesh) immediately prior to, or during, implantation of the spinal implant; (c) to the surface of the spinal implant and/or the tissue surrounding the implanted spinal implant (e.g., as an injectable, paste, gel, in situ forming gel or mesh) immediately after the implantation of the spinal implant; (d) by topical application of the composition into the anatomical space where the spinal implant may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the spinal implant as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device.

In one aspect, the subject polymer composition comprising an anti-scarring and/or anti-infective agent is infiltrated into the tissue adjacent to a spinal implant (e.g., an implantable cages or disc). In certain aspects, the spinal implant may be coated with (or adapted to contain) a fibrosis-inducing agent (e.g., silk or talc) on one part of the device and the subject polymer composition comprising an anti-scarring may be infiltrated into tissue adjacent to another part of the device. For example, the outer surface of the implant (e.g., a vertebral implant) may be coated with a fibrosis-inducing agent to improve adhesion between the device and the surrounding tissue, while the subject polymer composition comprising an anti-scarring may be infiltrated into tissue adjacent to the interior of the device to minimize adhesion of tissue to the interior of the implant. Examples of fibrosis-inducing agents and methods of using fibrosis-inducing agents in combination with spinal implants are described in co-pending application entitled, “Medical Implants and Fibrosis-Inducing Agents,” filed Nov. 20, 2003 (U.S. Ser. No. 60/524,023) and Jun. 9, 2004 (U.S. Ser. No. 60/578,471).

According to one aspect, any adhesion or fibrosis-inhibiting agent and/or anti-infective agent described above can be utilized in the practice of the present invention. In one aspect of the invention, the subject polymer compositions infiltrated into tissue adjacent to spinal implants may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As spinal implants are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

Neurostimulation Devices

In one aspect, the subject polymer compositions may be infiltrated into tissue adjacent to a neurostimulation device where a pulse generator delivers an electrical impulse to a nervous tissue (e.g., CNS, peripheral nerves, autonomic nerves) in order to regulate its activity. The subject polymer compositions may contain a therapeutic agent (e.g., an anti-scarring and/or anti-infective agent).

There are numerous neurostimulator devices where the occurrence of a fibrotic reaction may adversely affect the functioning of the device or the biological problem for which the device was implanted or used. Typically, fibrotic encapsulation of the electrical lead (or the growth of fibrous tissue between the lead and the target nerve tissue) slows, impairs, or interrupts electrical transmission of the impulse from the device to the tissue. This can cause the device to function suboptimally or not at all, or can cause excessive drain on battery life because increased energy is required to overcome the electrical resistance imposed by the intervening scar (or glial) tissue. Implantation of a neurostimulation device may also introduce or promote infection in the vicinity of the implant site.

Neurostimulation devices are used as alternative or adjunctive therapy for chronic, neurodegenerative diseases, which are typically treated with drug therapy, invasive therapy, or behavioral/lifestyle changes. Neurostimulation may be used to block, mask, or stimulate electrical signals in the body to treat dysfunctions, including, without limitation, pain, seizures, anxiety disorders, depression, ulcers, deep vein thrombosis, muscular atrophy, obesity, joint stiffness, muscle spasms, osteoporosis, scoliosis, spinal disc degeneration, spinal cord injury, deafness, urinary dysfunction and gastroparesis. Neurostimulation may be delivered to many different parts of the nervous system, including, spinal cord, brain, vagus nerve, sacral nerve, gastric nerve, auditory nerves, as well as organs, bone, muscles and tissues. As such, neurostimulators are developed to conform to the different anatomical structures and nervous system characteristics. Representative examples of neurologic and neurosurgical implants and devices, which may benefit from having the subject polymer composition infiltrated into adjacent tissue according to the present invention, include, e.g., nerve stimulator devices to provide pain relief, devices for continuous subarachnoid infusions, implantable electrodes, stimulation electrodes, implantable pulse generators, electrical leads, stimulation catheter leads, neurostimulation systems, electrical stimulators, cochlear implants, auditory stimulators and microstimulators.

Neurostimulation devices may also be classified based on their source of power, which includes: battery powered, radio-frequency (RF) powered, or a combination of both types. For battery powered neurostimulators, an implanted, non-rechargeable battery is used for power. The battery and leads are all surgically implanted and thus the neurostimulation device is completely internal. The settings of the totally implanted neurostimulator are controlled by the patient through an external magnet. The lifetime of the implant is generally limited by the duration of battery life and ranges from two to four years depending upon usage and power requirements. For RF-powered neurostimulation devices, the radio-frequency is transmitted from an externally worn source to an implanted passive receiver. Since the power source is readily rechargeable or replaceable, the radio-frequency system enables greater power resources and thus, multiple leads may be used in these systems. Specific examples include a neurostimulator that has a battery power source contained within to supply power over an eight hour period in which power may be replenished by an external radio frequency coupled device (See e.g., U.S. Pat. No. 5,807,397) or a microstimulator which is controlled by an external transmitter using data signals and powered by radio frequency (See e.g., U.S. Pat. No. 6,061,596).

Examples of commercially available neurostimulation products include a radio-frequency powered neurostimulator comprised of the 3272 MATTRIX Receiver, 3210 MATTRIX Transmitter and 3487A PISCES-QUAD Quadripolar Leads made by Medtronic, Inc. (Minneapolis, Minn.). Medtronic also sells a battery-powered ITREL 3 Neurostimulator and SYNERGY Neurostimulator, the INTERSIM Therapy for sacral nerve stimulation for urinary control, and leads such as the 3998 SPECIFY Lead and 3587A RESUME II Lead.

Another example of a neurostimulation device is a gastric pacemaker, in which multiple electrodes are positioned along the GI tract to deliver a phased electrical stimulation to pace peristaltic movement of the material through the GI tract. See, e.g., U.S. Pat. No. 5,690,691. A representative example of a gastric stimulation device is the ENTERRA Gastric Electrical Stimulation (GES) from Medtronic, Inc. (Minneapolis, Minn.).

The neurostimulation device, particularly the lead(s), must be positioned in a very precise manner to ensure that stimulation is delivered to the correct anatomical location in the nervous system. All, or parts, of a neurostimulation device can migrate following surgery, or excessive scar (or glial) tissue growth can occur around the implant, which can lead to a reduction in the efficacy of these devices (as described previously). Neurostimulation devices having the subject polymer compositions infiltrated into tissue adjacent to the electrode-tissue interface can be used to increase the efficacy and/or the duration of activity (particularly for fully-implanted, battery-powered devices) of the implant. Neurostimulation devices may also benefit from release of a therapeutic agent able to prevent or inhibit infection in the vicinity of the implant site. Accordingly, the present invention provides neurostimulator leads having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with neurostimulation devices have been described above.

Polymeric compositions may be infiltrated around implanted neurostimulation devices by applying the composition directly and/or indirectly into and/or onto (a) tissue adjacent to the neurostimulation device; (b) the vicinity of the neurostimulation device-tissue interface; (c) the region around the neurostimulation device; and (d) tissue surrounding the neurostimulation device. Methods for infiltrating the subject polymer compositions into tissue adjacent to a neurostimulation device include delivering the polymer composition: (a) to the surface of the neurostimulation device (e.g., as an injectable, paste, gel or mesh) during the implantation procedure; (b) to the surface of the tissue (e.g., as an injectable, paste, gel, in situ forming gel or mesh) immediately prior to, or during, implantation of the neurostimulation device; (c) to the surface of the neurostimulation device and/or the tissue surrounding the implanted neurostimulation device (e.g., as an injectable, paste, gel, in situ forming gel or mesh) immediately after the implantation of the neurostimulation device; (d) by topical application of the composition into the anatomical space where the neurostimulation device may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the neurostimulation device as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device, including the device only, lead only, electrode only and/or a combination thereof.

According to one aspect, any fibrosis-inhibiting and/or anti-infective agent described above may be utilized in the practice of the present invention. In one aspect of the invention, the subject polymer compositions infiltrated into tissue adjacent to neurostimulation devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As neurostimulation devices are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

For greater clarity, several specific neurostimulation devices and treatments will be described in greater detail below.

(1) Neurostimulation for the Treatment of Chronic Pain

In one aspect, the subject polymer compositions may be infiltrated into tissue adjacent to a neurostimulation device for the management of chronic pain. The subject polymer compositions may contain a therapeutic agent (e.g., an anti-scarring and/or anti-infective agent).

Chronic pain is one of the most important clinical problems in all of medicine. For example, it is estimated that over 5 million people in the United States are disabled by back pain. The economic cost of chronic back pain is enormous, resulting in over 100 million lost work days annually at an estimated cost of $50-100 billion. It has been reported that approximately 40 million Americans are afflicted with recurrent headaches and that the cost of medications for this condition exceeds $4 billion a year. A further 8 million people in the U.S. report that they experience chronic neck or facial pain and spend an estimated $2 billion a year for treatment. The cost of managing pain for oncology patients is thought to approach $12 billion. Chronic pain disables more people than cancer or heart disease and costs the American public more than both cancer and heart disease combined. In addition to the physical consequences, chronic pain has numerous other costs including loss of employment, marital discord, depression and prescription drug addiction. It goes without saying, therefore, that reducing the morbidity and costs associated with persistent pain remains a significant challenge for the healthcare system.

Intractable severe pain resulting from injury, illness, scoliosis, spinal disc degeneration, spinal cord injury, malignancy, arachnoiditis, chronic disease, pain syndromes (e.g., failed back syndrome, complex regional pain syndrome) and other causes is a debilitating and common medical problem. In many patients, the continued use of analgesics, particularly drugs like narcotics, are not a viable solution due to tolerance, loss of effectiveness, and addiction potential. In an effort to combat this, neurostimulation devices have been developed to treat severe intractable pain that is resistant to other traditional treatment modalities such as drug therapy, invasive therapy (surgery), or behavioral/lifestyle changes.

In principle, neurostimulation works by delivering low voltage electrical stimulation to the spinal cord or a particular peripheral nerve in order to block the sensation of pain. The Gate Control Theory of Pain (Ronald Meizack and Patrick Wall) hypothesizes that there is a “gate” in the dorsal horn of the spinal cord that controls the flow of pain signals from the peripheral receptors to the brain. It is speculated that the body can inhibit the pain signals (“close the gate”) by activating other (non-pain) fibers in the region of the dorsal horn. Neurostimulation devices are implanted in the epidural space of the spinal cord to stimulate non-noxious nerve fibers in the dorsal horn and mask the sensation of pain. As a result the patient typically experiences a tingling sensation (known as paresthesia) instead of pain. With neurostimulation, the majority of patients will report improved pain relief (50% reduction), increased activity levels and a reduction in the use of narcotics.

Pain management neurostimulation systems consist of a power source that generates the electrical stimulation, leads (typically 1 or 2) that deliver electrical stimulation to the spinal cord or targeted peripheral nerve, and an electrical connection that connects the power source to the leads. Neurostimulation systems can be battery powered, radio-frequency powered, or a combination of both. In general, there are two types of neurostimulation devices: those that are surgically implanted and are completely internal (i.e., the battery and leads are implanted), and those with internal (leads and radio-frequency receiver) and external (power source and antenna) components. For internal, battery-powered neurostimulators, an implanted, non-rechargeable battery and the leads are all surgically implanted. The settings of the totally implanted neurostimulator may be controlled by the host by using an external magnet and the implant has a lifespan of two to four years. For radio-frequency powered neurostimulators, the radio-frequency is transmitted from an externally worn source to an implanted passive receiver. The radio-frequency system enables greater power resources and thus, multiple leads may be used.

There are numerous neurostimulation devices that can be used for spinal cord stimulation in the management of pain control, postural positioning and other disorders. Examples of specific neurostimulation devices include those composed of a sensor that detects the position of the spine and a stimulator that automatically emits a series of pulses which decrease in amplitude when back is in a supine position. See e.g., U.S. Pat. Nos. 5,031,618 and 5,342,409. The neurostimulator may be composed of electrodes and a control circuit which generates pulses and rest periods based on intervals corresponding to the body's activity and regeneration period as a treatment for pain. See e.g., U.S. Pat. No. 5,354,320. The neurostimulator, which may be implanted within the epidural space parallel to the axis of the spinal cord, may transmit data to a receiver which generates a spinal cord stimulation pulse that may be delivered via a coupled, multi-electrode. See e.g., U.S. Pat. No. 6,609,031. The neurostimulator may be a stimulation catheter lead with a sheath and at least three electrodes that provide stimulation to neural tissue. See e.g., U.S. Pat. No. 6,510,347. The neurostimulator may be a self-centering epidual spinal cord lead with a pivoting region to stabilize the lead which inflates when injected with a hardening agent. See e.g., U.S. Pat. No. 6,308,103. Other neurostimulators used to induce electrical activity in the spinal cord are described in, e.g., U.S. Pat. Nos. 6,546,293; 6,236,892; 4,044,774 and 3,724,467.

Neurostimulation devices for the management of chronic pain, which may benefit from having the subject polymer composition infiltrated into adjacent tissue according to the present invention, include commercially available products. Commercially available neurostimulation devices for the management of chronic pain include the SYNERGY, INTREL, X-TREL and MATTRIX neurostimulation systems from Medtronic, Inc. The percutaneous leads in this system can be quadripolar (4 electrodes), such as the PISCES-QUAD, PISCES-QUAD PLUS and the PISCES-QUAD Compact, or octapolar (8 electrodes) such as the OCTAD lead. The surgical leads themselves are quadripolar, such as the SPECIFY Lead, the RESUME II Lead, the RESUME TL Lead and the ON-POINT PNS Lead, to create multiple stimulation combinations and a broad area of paresthesia. These neurostimulation systems and associated leads may be described, for example, in U.S. Pat. Nos. 6,671,544; 6,654,642; 6,360,750; 6,353,762; 6,058,331; 5,342,409; 5,031,618 and 4,044,774. Neurostimulating leads such as these may benefit from release of a therapeutic agent able to reducing scarring at the electrode-tissue interface to increase the efficiency of impulse transmission and increase the duration that the leads function clinically. Nuerostimulating leads such as these may also benefit from release of a therapeutic agent able to prevent or inhibit infection in the vicinity of the implant site. In one aspect, the device includes neurostimulation devices for the management of chronic pain and/or leads having the subject polymer composition comprising an anti-scarring agent and/or anti-infective agent infiltrated into tissue adjacent to where the device and/or leads are or will be implanted. In another aspect, the present invention provides leads having the subject polymer composition comprising an anti-scarring agent and/or anti-infective agent infiltrated into tissue adjacent to the epidural space where the lead is or will be implanted. Other commercially available systems that may useful for the practice of this invention as described above include the rechargeable PRECISION Spinal Cord Stimulation System (Advanced Bionics Corporation, Sylmar, Calif.; which is a Boston Scientific Company) which can drive up to 16 electrodes (see e.g., U.S. Pat. No. 6,735,474; 6,735,475; 6,659,968; 6,622,048; 6,516,227 and 6,052,624). The GENESIS XP Spinal Cord Stimulator available from Advanced Neuromodulation Systems, Inc. (Plano, Tex.; see e.g., U.S. Pat. Nos. 6,748,276; 6,609,031 and 5,938,690) as well as the Vagus Nerve Stimulation (VNS) Therapy System available from Cyberonics, Inc. (Houston, Tex.; see e.g., U.S. Pat. Nos. 6,721,603 and 5,330,515) may also benefit from having the subject polymer composition infiltrated into adjacent tissue according to the present invention.

Regardless of the specific design features, for neurostimulation to be effective in pain relief, the leads must be accurately positioned adjacent to the portion of the spinal cord or the targeted peripheral nerve that is to be electrically stimulated. Neurostimulators can migrate following surgery or excessive tissue growth or extracellular matrix deposition can occur around neurostimulators, which can lead to a reduction in the functioning of these devices. Neurostimulation devices having the subject polymer compositions infiltrated into tissue adjacent to the electrode-tissue interface can be used to increase the duration that these devices clinically function. Neurostimulation devices may also benefit from release of a therapeutic agent able to prevent or inhibit infection in the vicinity of the implant site. In one aspect, the present invention provides neurostimulation devices for the management of chronic pain having the subject polymer compositions infiltrated into tissue adjacent to the implanted portion (particularly the leads), where the subject polymer compositions may include a therapeutic agent (e.g., an anti-scarring and/or anti-infective agent). Numerous polymeric and non-polymeric delivery systems for use in connection with neurostimulation devices for the management of chronic pain have been described above.

Polymeric compositions may be infiltrated around implanted neurostimulation devices for the management of chronic pain by applying the composition directly and/or indirectly into and/or onto (a) tissue adjacent to the neurostimulation device for the management of chronic pain; (b) the vicinity of the neurostimulation device for the management of chronic pain-tissue interface; (c) the region around the neurostimulation device for the management of chronic pain; and (d) tissue surrounding the neurostimulation device for the management of chronic pain. Methods for infiltrating the subject polymer compositions into tissue adjacent to a neurostimulation device for the management of chronic pain include delivering the polymer composition: (a) to the surface of the neurostimulation device for the management of chronic pain (e.g., as an injectable, paste, gel or mesh) during the implantation procedure; (b) to the surface of the tissue (e.g., as an injectable, paste, gel, in situ forming gel or mesh) immediately prior to, or during, implantation of the neurostimulation device for the management of chronic pain; (c) to the surface of the neurostimulation device for the management of chronic pain and/or the tissue surrounding the implanted neurostimulation device for the management of chronic pain (e.g., as an injectable, paste, gel, in situ forming gel or mesh) immediately after the implantation of the neurostimulation device for the management of chronic pain; (d) by topical application of the composition into the anatomical space where the neurostimulation device for the management of chronic pain may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the neurostimulation device for the management of chronic pain as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device, including the device only, lead only, electrode only and/or a combination thereof.

According to one aspect, any fibrosis-inhibiting and/or anti-infective agent described above may be utilized in the practice of the present invention. In one aspect of the invention, the subject polymer compositions infiltrated into tissue adjacent to neurostimulation devices for the management of chronic pain may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As neurostimulation devices for the management of chronic pain are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

(2) Neurostimulation for the Treatment of Parkinson's Disease

In one aspect, the subject polymer compositions may be infiltrated into tissue adjacent to a neurostimulation device for the treatment of Parkinson's disease. The subject polymer compositions may contain a therapeutic agent (e.g., an anti-scarring and/or anti-infective agent).

Neurostimulation devices implanted into the brain are used to control the symptoms associated with Parkinson's disease or essential tremor. Typically, these are dual chambered stimulator devices (similar to cardiac pacemakers) that deliver bilateral stimulation to parts of the brain that control motor function. Electrical stimulation is used to relieve muscular symptoms due to Parkinson's disease itself (tremor, rigidity, bradykinesia, akinesia) or symptoms that arise as a result of side effects of the medications used to treat the disease (dyskinesias). Two stimulating electrodes are implanted in the brain (usually bilaterally in the subthalamic nucleus or the globus pallidus interna) for the treatment of levodopa-responsive Parkinson's and one is implanted (in the ventral intermediate nucleus of the thalamus) for the treatment of tremor. The electrodes are implanted in the brain by a functional stereotactic neurosurgeon using a stereotactic head frame and MRI or CT guidance. The electrodes are connected via extensions (which run under the skin of the scalp and neck) to a neurostimulatory (pulse generating) device implanted under the skin near the clavicle. A neurologist can then optimize symptom control by adjusting stimulation parameters using a noninvasive control device that communicates with the neurostimulator via telemetry. The patient is also able to turn the system on and off using a magnet and control the device (within limits set by the neurologist) settings using a controller device. This form of deep brain stimulation has also been investigated for the treatment pain, epilepsy, psychiatric conditions (obsessive-compulsive disorder) and dystonia.

Several devices have been described for such applications including, for example, a neurostimulator and an implantable electrode that has a flexible, non-conducting covering material, which is used for tissue monitoring and stimulation of the cortical tissue of the brain as well as other tissue. See e.g., U.S. Pat. No. 6,024,702. The neurostimulator (pulse generator) may be an intracranially implanted electrical control module and a plurality of electrodes which stimulate the brain tissue with an electrical signal at a defined frequency. See e.g., U.S. Pat. No.6,591,138. The neurostimulator may be a system composed of at least two electrodes adapted to the cranium and a control module adapted to be implanted beneath the scalp for transmitting output electrical signals and also external equipment for providing two-way communication. See e.g., U.S. Pat. No. 6,016,449. The neurostimulator may be an implantable assembly composed of a sensor and two electrodes, which are used to modify the electrical activity in the brain. See e.g., U.S. Pat. No. 6,466,822.

Neurostimulation devices for the treatment of Parkinson's disease, which may benefit from having the subject polymer composition infiltrated into adjacent tissue according to the present invention, include commercially available products. A commercial example of a device used to treat Parkinson's disease and essential tremor includes the ACTIVA System by Medtronic, Inc. (see, for example, U.S. Pat. Nos. 6,671,544 and 6,654,642). This system consists of the KINETRA Dual Chamber neurostimulator, the SOLETRA neurostimulator or the INTREL neurostimulator, connected to an extension (an insulated wire), that is further connected to a DBS lead. The DBS lead consists of four thin, insulated, coiled wires bundled with polyurethane. Each of the four wires ends in a 1.5 mm long electrode. In one aspect, the present invention provides neurostimulation devices for the treatment of Parkinson's disease having the subject polymer compositions infiltrated into tissue adjacent to where the device and/or leads are or will be implanted, where the subject polymer compositions may include a therapeutic agent (e.g., an anti-scarring and/or anti-infective agent). In another aspect, the present invention provides leads (e.g., DBS leads) having the subject polymer composition comprising an anti-scarring agent and/or anti-infective agent infiltrated into tissue adjacent to the tissue where the lead is or will be implanted. In another aspect, the present invention provides DBS leads having the subject polymer composition comprising an anti-scarring agent and/or anti-infective agent infiltrated into the brain tissue adjacent to where the electrodes of the leads are or will be implanted.

Numerous polymeric and non-polymeric delivery systems for use in connection with neurostimulation devices for the treatment of Parkinson's disease have been described above.

Polymeric compositions may be infiltrated around implanted neurostimulation devices for the treatment of Parkinson's disease by applying the composition directly and/or indirectly into and/or onto (a) tissue adjacent to the neurostimulation device for the treatment of Parkinson's disease; (b) the vicinity of the neurostimulation device for the treatment of Parkinson's disease-tissue interface; (c) the region around the neurostimulation device for the treatment of Parkinson's disease; and (d) tissue surrounding the neurostimulation device for the treatment of Parkinson's disease. Methods for infiltrating the subject polymer compositions into tissue adjacent to a neurostimulation device for the treatment of Parkinson's disease include delivering the polymer composition: (a) to the surface of the neurostimulation device for the treatment of Parkinson's disease (e.g., as an injectable, paste, gel or mesh) during the implantation procedure; (b) to the surface of the tissue (e.g., as an injectable, paste, gel, in situ forming gel or mesh) immediately prior to, or during, implantation of the neurostimulation device for the treatment of Parkinson's disease; (c) to the surface of the neurostimulation device for the treatment of Parkinson's disease and/or the tissue surrounding the implanted neurostimulation device for the treatment of Parkinson's disease (e.g., as an injectable, paste, gel, in situ forming gel or mesh) immediately after the implantation of the neurostimulation device for the treatment of Parkinson's disease; (d) by topical application of the composition into the anatomical space where the neurostimulation device for the treatment of Parkinson's disease may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the neurostimulation device for the treatment of Parkinson's disease as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device, including the device only, lead only, electrode only and/or a combination thereof.

According to one aspect, any fibrosis-inhibiting and/or anti-infective agent described above may be utilized in the practice of the present invention. In one aspect of the invention, the subject polymer compositions infiltrated into tissue adjacent to neurostimulation devices for the treatment of Parkinson's disease may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As neurostimulation devices for the treatment of Parkinson's disease are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

(3) Vagal Nerve Stimulation for the Treatment of Epilepsy

In one aspect, the subject polymer compositions may be infiltrated into tissue adjacent to a neurostimulation device for the treatment of epilepsy. The subject polymer compositions may contain a therapeutic agent (e.g., an anti-scarring and/or anti-infective agent).

Neurostimulation devices are also used for vagal nerve stimulation in the management of pharmacoresistant epilepsy (i.e., epilepsy that is uncontrolled despite appropriate medical treatment with ant-epileptic drugs). Approximately 30% of epileptic patients continue to have seizures despite of multiple attempts at controlling the disease with drug therapy or are unable to tolerate the side effects of their medications. It is estimated that approximately 2.5 million patients in the United States suffer from treatment-resistant epilepsy and may benefit from vagal nerve stimulation therapy. As such, inadequate seizure control remains a significant medical problem with many patients suffering from diminished self esteem, poor academic achievement and a restricted lifestyle as a result of their illness.

The vagus nerve (also called the 10^{th} cranial nerve) contains primarily afferent sensory fibres that carry information from the neck, thorax and abdomen to the nucleus tractus soltarius of the brainstem and on to multiple noradrenergic and serotonergic neuromodulatory systems in the brain and spinal cord. Vagal nerve stimulation (VNS) has been shown to induce progressive EEG changes, alter bilateral cerebral blood flow, and change blood flow to the thalamus. Although the exact mechanism of seizure control is not known, VNS has been demonstrated clinically to terminate seizures after seizure onset, reduce the severity and frequency of seizures, prevent seizures when used prophylactically over time, improve quality of life, and reduce the dosage, number and side effects of anti-epileptic medications (resulting in improved alertness, mood, memory).

In VNS, a bipolar electrical lead is surgically implanted such that it transmits electrical stimulation from the pulse generator to the left vagus nerve in the neck. The pulse generator is an implanted, lithium carbon monofluoride battery-powered device that delivers a precise pattern of stimulation to the vagus nerve. The pulse generator can be programmed (using a programming wand) by the neurologist to suit an individual patient's symptoms, while the patient can turn the device on and off through the use of an external magnet. Chronic electrical stimulation which can be used as a direct treatment for epilepsy is described in, for example, U.S. Pat. No. 6,016,449, whereby, an implantable neurostimulator is coupled to relatively permanent deep brain electrodes. The implantable neurostimulator may be composed of an implantable electrical lead having a furcated, or split, distal portion with two or more separate end segments, each of which bears at least one sensing or stimulation electrode, which may be used to treat epilepsy and other neurological disorders. See e.g., U.S. Pat. No. 6,597,953.

Neurostimulation devices for the treatment of epilepsy, which may benefit from having the subject polymer composition infiltrated into adjacent tissue according to the present invention, include commercially available products. A commercial example of a VNS system is the product produced by Cyberonics, Inc. that includes the Model 300 and Model 302 leads, the Model 101 and Model 102R pulse generators, the Model 201 programming wand and Model 250 programming software, and the Model 220 magnets. These products manufactured by Cyberonics, Inc. may be described, for example, in U.S. Pat. Nos. 5,540,730 and 5,299,569.

Regardless of the specific design features, for vagal nerve stimulation to be effective in epilepsy, the leads must be accurately positioned adjacent to the left vagus nerve. If excessive scar tissue growth or extracellular matrix deposition occurs around the VNS leads, this can reduce the efficacy of the device. VNS devices having the subject polymer compositions infiltrated into tissue adjacent can increase the efficiency of impulse transmission and increase the duration that these devices function clinically. VNS devices may also benefit from release of a therapeutic agent able to prevent or inhibit infection in the vicinity of the implant site. In one aspect, the device includes VNS devices and/or leads having the subject polymer composition comprising an anti-scarring agent and/or anti-infective agent infiltrated into tissue adjacent to where the VNS device and/or leads are or will be implanted. In another aspect, the present invention provides leads having the subject polymer composition comprising an anti-scarring agent and/or anti-infective agent infiltrated into tissue adjacent to the vagus nerve where the lead will be implanted.

In another aspect, the present invention provides neurostimulation devices for the treatment of epilepsy having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with neurostimulation devices for the treatment of epilepsy have been described above.

Polymeric compositions may be infiltrated around implanted neurostimulation devices for the treatment of epilepsy by applying the composition directly and/or indirectly into and/or onto (a) tissue adjacent to the neurostimulation device for the treatment of epilepsy; (b) the vicinity of the neurostimulation device for the treatment of epilepsy-tissue interface; (c) the region around the neurostimulation device for the treatment of epilepsy; and (d) tissue surrounding the neurostimulation device for the treatment of epilepsy. Methods for infiltrating the subject polymer compositions into tissue adjacent to a neurostimulation device for the treatment of epilepsy include delivering the polymer composition: (a) to the surface of the neurostimulation device for the treatment of epilepsy (e.g., as an injectable, paste, gel or mesh) during the implantation procedure; (b) to the surface of the tissue (e.g., as an injectable, paste, gel, in situ forming gel or mesh) immediately prior to, or during, implantation of the neurostimulation device for the treatment of epilepsy; (c) to the surface of the neurostimulation device for the treatment of epilepsy and/or the tissue surrounding the implanted neurostimulation device for the treatment of epilepsy (e.g., as an injectable, paste, gel, in situ forming gel or mesh) immediately after the implantation of the neurostimulation device for the treatment of epilepsy; (d) by topical application of the composition into the anatomical space where the neurostimulation device for the treatment of epilepsy may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the neurostimulation device for the treatment of epilepsy as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device, including the device only, lead only, electrode only and/or a combination thereof.

According to one aspect, any fibrosis-inhibiting and/or anti-infective agent described above may be utilized in the practice of the present invention. In one aspect of the invention, the subject polymer compositions infiltrated into tissue adjacent to neurostimulation devices for the treatment of epilepsy may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As neurostimulation devices for the treatment of epilepsy are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

(4) Vagal Nerve Stimulation for the Treatment of Other Disorders

In one aspect, the subject polymer compositions may be infiltrated into tissue adjacent to a neurostimulation device for the treatment of neurological disorders. The subject polymer compositions may contain a therapeutic agent (e.g., an anti-scarring and/or anti-infective agent).

It was discovered during the use of VNS for the treatment of epilepsy that some patients experienced an improvement in their mood during therapy. As such, VNS is currently being examined for use in the management of treatment-resistant mood disorders such as depression and anxiety. Depression remains an enormous clinical problem in the Western World with over 1% (25 million people in the United States) suffering from depression that is inadequately treated by pharmacotherapy. Vagal nerve stimulation has been examined in the management of conditions such as anxiety (panic disorder, obsessive-compulsive disorder, post-traumatic stress disorder), obesity, migraine, sleep disorders, dementia, Alzheimer's disease and other chronic or degenerative neurological disorders. VNS has also been examined for use in the treatment of medically significant obesity.

The implantable neurostimulator for the treatment of neurological disorders may be composed of an implantable electrical lead having a furcated, or split, distal portion with two or more separate end segments, each of which bears at least one sensing or stimulation electrode. See e.g., U.S. Pat. No. 6,597,953. The implantable neurostimulator may be an apparatus for treating Alzheimer's disease and dementia, particularly for neuro modulating or stimulating left vagus nerve, composed of an implantable lead-receiver, external stimulator, and primary coil. See e.g., U.S. Pat. No. 6,615,085.

Neurostimulation devices for the treatment of neurological disorders, which may benefit from having the subject polymer composition infiltrated into adjacent tissue according to the present invention, include commercially available products. Cyberonics, Inc. manufactures the commercially available VNS system, including the Model 300 and Model 302 leads, the Model 101 and Model 102R pulse generators, the Model 201 programming wand and Model 250 programming software, and the Model 220 magnets. These products as well as others that are being developed by Cyberonics, Inc. may be used to treat neurological disorders, including depression (see e.g., U.S. Pat. No. 5,299,569), dementia (see e.g., U.S. Pat. No. 5,269,303), migraines (see e.g., U.S. Pat. No. 5,215,086), sleep disorders (see e.g., U.S. Pat. No. 5,335,657) and obesity (see e.g., U.S. Pat. Nos. 6,587,719; 6,609,025; 5,263,480 and 5,188,104).

It is important to note that the fundamentals of treatment are identical to those described above for epilepsy. The devices employed and the principles of therapy are also similar. As was described above for the treatment of epilepsy, if excessive scar tissue growth or extracellular matrix deposition occurs around the VNS leads, this can reduce the efficacy of the device. VNS devices may benefit from release of a therapeutic agent able to reducing scarring at the electrode-tissue interface to increase the efficiency of impulse transmission and increase the duration that these devices function clinically for the treatment of depression, anxiety, obesity, sleep disorders and dementia. VNS devices may also benefit from release of a therapeutic agent able to prevent or inhibit infection in the vicinity of the implant site. In one aspect, the device includes VNS devices and/or leads having the subject polymer composition comprising an anti-scarring agent and/or anti-infective agent infiltrated into tissue adjacent to where the VNS device and/or leads are or will be implanted. In another aspect, the present invention provides leads having the subject polymer composition comprising an anti-scarring agent and/or anti-infective agent infiltrated into tissue adjacent to the vagus nerve where the lead will be implanted.

In another aspect, the present invention provides neurostimulation devices for the treatment of neurological disorders having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with neurostimulation devices for the treatment of neurological disorders have been described above.

Polymeric compositions may be infiltrated around implanted neurostimulation devices for the treatment of neurological disorders by applying the composition directly and/or indirectly into and/or onto (a) tissue adjacent to the neurostimulation device for the treatment of neurological disorders; (b) the vicinity of the neurostimulation device for the treatment of neurological disorders-tissue interface; (c) the region around the neurostimulation device for the treatment of neurological disorders; and (d) tissue surrounding the neurostimulation device for the treatment of neurological disorders. Methods for infiltrating the subject polymer compositions into tissue adjacent to a neurostimulation device for the treatment of neurological disorders include delivering the polymer composition: (a) to the surface of the neurostimulation device for the treatment of neurological disorders (e.g., as an injectable, paste, gel or mesh) during the implantation procedure; (b) to the surface of the tissue (e.g., as an injectable, paste, gel, in situ forming gel or mesh) immediately prior to, or during, implantation of the neurostimulation device for the treatment of neurological disorders; (c) to the surface of the neurostimulation device for the treatment of neurological disorders and/or the tissue surrounding the implanted neurostimulation device for the treatment of neurological disorders (e.g., as an injectable, paste, gel, in situ forming gel or mesh) immediately after the implantation of the neurostimulation device for the treatment of neurological disorders; (d) by topical application of the composition into the anatomical space where the neurostimulation device for the treatment of neurological disorders may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the neurostimulation device for the treatment of neurological disorders as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device, including the device only, lead only, electrode only and/or a combination thereof.

According to one aspect, any fibrosis-inhibiting and/or anti-infective agent described above may be utilized in the practice of the present invention. In one aspect of the invention, the subject polymer compositions infiltrated into tissue adjacent to neurostimulation devices for the treatment of neurological disorders may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As neurostimulation devices for the treatment of neurological disorders are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

(5) Sacral Nerve Stimulation for Bladder Control Problems

In one aspect, the subject polymer compositions may be infiltrated into tissue adjacent to a neurostimulation system to treat bladder conditions. The subject polymer compositions may contain a therapeutic agent (e.g., an anti-scarring and/or anti-infective agent).

Sacral nerve stimulation is used in the management of patients with urinary control problems such as urge incontinence, nonobstructive urinary retention, or urgency-frequency. Millions of people suffer from bladder control problems and a significant percentage (estimated to be in excess of 60%) is not adequately treated by other available therapies such as medications, absorbent pads, external collection devices, bladder augmentation or surgical correction. This can be a debilitating medical problem that can cause severe social anxiety and cause people to become isolated and depressed.

Mild electrical stimulation of the sacral nerve is used to influence the functioning of the bladder, urinary sphincter, and the pelvic floor muscles (all structures which receive nerve supply from the sacral nerve). An electrical lead is surgically implanted adjacent to the sacral nerve and a neurostimulator is implanted subcutaneously in the upper buttock or abdomen; the two are connected by an extension. The use of tined leads allows sutureless anchoring of the leads and minimally-invasive placement of the leads under local anesthesia. A handheld programmer is available for adjustment of the device by the attending physician and a patient-controlled programmer is available to adjust the settings and to turn the device on and off. The pulses are adjusted to provide bladder control and relieve the patient's symptoms.

Several neurostimulation systems have been described for sacral nerve stimulation in which electrical stimulation is targeted towards the bladder, pelvic floor muscles, bowel and/or sexual organs. For example, the neurostimulator may be an electrical stimulation system composed of an electrical stimulator and leads having insulator sheaths, which may be anchored in the sacrum using minimally-invasive surgery. See e.g., U.S. Pat. No. 5,957,965. In another aspect, the neurostimulator may be used to condition pelvic, sphincter or bladder muscle tissue. For example, the neurostimulator may be intramuscular electrical stimulator composed of a pulse generator and an elongated medical lead that is used for electrically stimulating or sensing electrical signals originating from muscle tissue. See e.g., U.S. Pat. No. 6,434,431. Another neurostimulation system consists of a leadless, tubular-shaped microstimulator that is implanted at pelvic floor muscles or associated nerve tissue that need to be stimulated to treat urinary incontinence. See e.g., U.S. Pat. No. 6,061,596.

Neurostimulation systems to treat bladder conditions, which may benefit from having the subject polymer composition infiltrated into adjacent tissue according to the present invention, include commercially available products. A commercially available example of a neurostimulation system to treat bladder conditions is the INTERSTIM Sacral Nerve Stimulation System made by Medtronic, Inc. See e.g., U.S. Pat. Nos. 6,104,960; 6,055,456 and 5,957,965.

Regardless of the specific design features, for bladder control therapy to be effective, the leads must be accurately positioned adjacent to the sacral nerve, bladder, sphincter or pelvic muscle (depending upon the particular system employed). If excessive scar tissue growth or extracellular matrix deposition occurs around the leads, efficacy can be compromised. Sacral nerve stimulating devices (such as INTERSTIM) having the subject polymer compositions infiltrated into tissue adjacent to the electrode-tissue interface can increase the efficiency of impulse transmission and increase the duration that these devices function clinically. Nuerostimulating devices such as these may also benefit from release of a therapeutic agent able to prevent or inhibit infection in the vicinity of the implant site. In one aspect, the device includes sacral nerve stimulating devices and/or leads having the subject polymer composition comprising an anti-scarring agent and/or anti-infective agent infiltrated into tissue adjacent to where the sacral nerve stimulating device and/or leads are or will be implanted. In another aspect, the present invention provides leads having the subject polymer composition comprising an anti-scarring agent and/or anti-infective agent infiltrated into tissue adjacent to the sacral nerve where the lead will be implanted.

For devices designed to stimulate the bladder or pelvic muscle tissue directly, slightly different embodiments may be required. In this aspect, the device includes bladder or pelvic muscle stimulating devices, leads, and/or sensors having the subject polymer composition comprising an anti-scarring agent and/or anti-infective agent infiltrated into tissue adjacent to where the sacral nerve stimulating device and/or leads are or will be implanted In another aspect, the present invention provides leads and/or sensors, which are delivering an impulse or monitoring the activity of the muscle, having the subject polymer composition comprising an anti-scarring agent and/or anti-infective agent infiltrated into tissue adjacent to the tissue (e.g., muscle) where the lead and/or sensor will be implanted.

In another aspect, the present invention provides neurostimulation systems to treat bladder conditions having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with neurostimulation systems to treat bladder conditions have been described above.

Polymeric compositions may be infiltrated around implanted neurostimulation systems to treat bladder conditions by applying the composition directly and/or indirectly into and/or onto (a) tissue adjacent to the neurostimulation system to treat bladder conditions; (b) the vicinity of the neurostimulation system to treat bladder conditions-tissue interface; (c) the region around the neurostimulation system to treat bladder conditions; and (d) tissue surrounding the neurostimulation system to treat bladder conditions. Methods for infiltrating the subject polymer compositions into tissue adjacent to a neurostimulation system to treat bladder conditions include delivering the polymer composition: (a) to the surface of the neurostimulation system to treat bladder conditions (e.g., as an injectable, paste, gel or mesh) during the implantation procedure; (b) to the surface of the tissue (e.g., as an injectable, paste, gel, in situ forming gel or mesh) immediately prior to, or during, implantation of the neurostimulation system to treat bladder conditions; (c) to the surface of the neurostimulation system to treat bladder conditions and/or the tissue surrounding the implanted neurostimulation system to treat bladder conditions (e.g., as an injectable, paste, gel, in situ forming gel or mesh) immediately after the implantation of the neurostimulation system to treat bladder conditions; (d) by topical application of the composition into the anatomical space where the neurostimulation system to treat bladder conditions may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the neurostimulation system to treat bladder conditions as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device, including the device only, lead only, electrode only and/or a combination thereof.

According to one aspect, any fibrosis-inhibiting and/or anti-infective agent described above may be utilized in the practice of the present invention. In one aspect of the invention, the subject polymer compositions infiltrated into tissue adjacent to neurostimulation systems to treat bladder conditions may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As neurostimulation systems to treat bladder conditions are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

(6) Gastric Nerve Stimulation for the Treatment of GI Disorders

In one aspect, the subject polymer compositions may be infiltrated into tissue adjacent to a device for treatment of GI disorders. The subject polymer compositions may contain a therapeutic agent (e.g., an anti-scarring and/or anti-infective agent).

Neurostimulator of the gastric nerve (which supplies the stomach and other portions of the upper GI tract) is used to influence gastric emptying and satiety sensation in the management of clinically significant obesity or problems associated with impaired GI motility. Morbid obesity has reached epidemic proportions and is thought to affect over 25 million Americans and lead to significant health problems such as diabetes, heart attack, stroke and death. Mild electrical stimulation of the gastric nerve is used to influence the functioning of the upper GI tract and stomach (all structures which receive nerve supply from the gastric nerve). An electrical lead is surgically implanted adjacent to the gastric nerve and a neurostimulator is implanted subcutaneously; the two are connected by an extension. A handheld programmer is available for adjustment of the device by the attending physician and a patient-controlled programmer is available to adjust the settings and to turn the device on and off. The pulses are adjusted to provide a sensation of satiety and relieve the sensation of hunger experienced by the patient. This can reduce the amount of food (and hence caloric) intake and allow the patient to lose weight successfully. Related devices include neurostimulation devices used to stimulate gastric emptying in patients with impaired gastric motility, a neurostimulator to promote bowel evacuation in patients with constipation (stimulation is delivered to the colon), and devices targeted at the bowel for patients with other GI motility disorders.

Several such devices have been described including, for example, a sensor that senses electrical activity in the gastrointestinal tract which is coupled to a pulse generator that emits and inhibits asynchronous stimulation pulse trains based on the natural gastrointestinal electrical activity. See e.g., U.S. Pat. No. 5,995,872. Other neurostimulation devices deliver impulses to the colon and rectum to manage constipation and are composed of electrical leads, electrodes and an implanted stimulation generator. See e.g., U.S. Pat. No. 6,026,326. The neurostimulator may be a pulse generator and electrodes that electrically stimulate the neuromuscular tissue of the viscera to treat obesity. See e.g., U.S. Pat. No. 6,606,523. The neurostimulator may be a hermetically sealed implantable pulse generator that is electrically coupled to the gastrointestinal tract and emits two rates of electrical stimulation to treat gastroparesis for patients with impaired gastric emptying. See e.g., U.S. Pat. No. 6,091,992. The neurostimulator may be composed of an electrical signal controller, connector wire and attachment lead which generates continuous low voltage electrical stimulation to the fundus of the stomach to control appetite. See e.g., U.S. Pat. No. 6,564,101. Other neurostimulators that are used to electrically stimulate the gastrointestinal tract are described in, e.g., U.S. Pat. Nos. 6,453,199; 6,449,511 and 6,243,607.

Devices for treatment of GI disorders, which may benefit from having the subject polymer composition infiltrated into adjacent tissue according to the present invention, include commercially available products. A commercially available example of a gastric nerve stimulation device for use with the present invention is the TRANSCEND Implantable Gastric Stimulator (IGS), which is currently being developed by Transneuronix, Inc. (Mt. Arlington, N.J.). The IGS is a programmable, bipolar pulse generator that delivers small bursts of electrical pulses through the lead to the stomach wall to treat obesity. See, e.g., U.S. Pat. Nos. 6,684,104 and 6,165,084.

Regardless of the specific design features, for gastric nerve stimulation to be effective in satiety control (or gastroparesis), the leads must be accurately positioned adjacent to the gastric nerve. If excessive scar tissue growth or extracellular matrix deposition occurs around the leads, efficacy can be compromised. Gastric nerve stimulating devices (and other implanted devices designed to influence GI motility) having the subject polymer compositions infiltrated into tissue adjacent to the electrode-tissue interface can increase the efficiency of impulse transmission and increase the duration that these devices function clinically. Gastric nerve stimulating devices (and other implanted devices designed to influence GI motility) may also benefit from release of a therapeutic agent able to prevent or inhibit infection in the vicinity of the implant site. In one aspect, the device includes gastric nerve stimulating devices and/or leads having the subject polymer composition comprising an anti-scarring agent and/or anti-infective agent infiltrated into tissue adjacent to where the gastric nerve stimulating device and/or leads are or will be implanted. In another aspect, the present invention provides leads having the subject polymer composition comprising an anti-scarring agent and/or anti-infective agent infiltrated into tissue adjacent to the gastric nerve where the lead will be implanted.

In another aspect, the present invention provides devices for treatment of GI disorders having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with devices for treatment of GI disorders have been described above.

Polymeric compositions may be infiltrated around implanted devices for treatment of GI disorders by applying the composition directly and/or indirectly into and/or onto (a) tissue adjacent to the device for treatment of GI disorders; (b) the vicinity of the device for treatment of GI disorders-tissue interface; (c) the region around the device for treatment of GI disorders; and (d) tissue surrounding the device for treatment of GI disorders. Methods for infiltrating the subject polymer compositions into tissue adjacent to a device for treatment of GI disorders include delivering the polymer composition: (a) to the surface of the device for treatment of GI disorders (e.g., as an injectable, paste, gel or mesh) during the implantation procedure; (b) to the surface of the tissue (e.g., as an injectable, paste, gel, in situ forming gel or mesh) immediately prior to, or during, implantation of the device for treatment of GI disorders; (c) to the surface of the device for treatment of GI disorders and/or the tissue surrounding the implanted device for treatment of GI disorders (e.g., as an injectable, paste, gel, in situ forming gel or mesh) immediately after the implantation of the device for treatment of GI disorders; (d) by topical application of the composition into the anatomical space where the device for treatment of GI disorders may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the device for treatment of GI disorders as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device, including the device only, lead only, electrode only and/or a combination thereof.

According to one aspect, any fibrosis-inhibiting and/or anti-infective agent described above may be utilized in the practice of the present invention. In one aspect of the invention, the subject polymer compositions infiltrated into tissue adjacent to devices for treatment of GI disorders may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As devices for treatment of GI disorders are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

(7) Cochlear Implants for the Treatment of Deafness

In one aspect, the subject polymer compositions may be infiltrated into tissue adjacent to a cochlear implant. The subject polymer compositions may contain a therapeutic agent (e.g., an anti-scarring and/or anti-infective agent).

Neurostimulation is also used in the form of a cochlear implant that stimulates the auditory nerve for correcting sensorineural deafness. A sound processor captures sound from the environment and processes it into a digital signal that is transmitted via an antenna through the skin to the cochlear implant. The cochlear implant, which is surgically implanted in the cochlea adjacent to the auditory nerve, converts the digital information into electrical signals that are communicated to the auditory nerve via an electrode array. Effectively, the cochlear implant serves to bypass the nonfunctional cochlear transducers and directly depolarize afferent auditory nerve fibers. This stimulates the nerve to send signals to the auditory center in the brain and allows the patient to “hear” the sounds detected by the sound processor. The treatment is used for adults with 70 dB or greater hearing loss (and able to understand up to 50% of words in a sentence using a hearing aid) or children 12 months or older with 90 dB hearing loss in both ears.

Although many implantations are performed without incident, approximately 12-15% of patients experience some complications. Histologic assessment of cochlear implants has revealed that several forms of injury and scarring can occur. Surgical trauma can induce cochlear fibrosis, cochlear neossification and injury to the membranous cochlea (including loss of the sensorineural elements). A foreign body reaction along the implant and the electrode can produce a fibrous tissue response along the electrode array that has been associated with implant failure. Implantation of a neurostimulation device may also introduce or promote infection in the vicinity of the implant site.

A variety of suitable cochlear implant systems or “bionic ears” have been described for use in association with this invention. For example, the neurostimulator may be composed of a plurality of transducer elements which detect vibrations and then generates a stimulus signal to a corresponding neuron connected to the cranial nerve. See e.g., U.S. Pat. No. 5,061,282. The neurostimulator may be a cochlear implant having a sound-to-electrical stimulation encoder, a body implantable receiver-stimulator and electrodes, which emit pulses based on received electrical signals. See e.g., U.S. Pat. No. 4,532,930. The neurostimulator may be an intra-cochlear apparatus that is composed of a transducer that converts an audio signal into an electrical signal and an electrode array which electrically stimulates predetermined locations of the auditory nerve. See e.g., U.S. Pat. No. 4,400,590. The neurostimulator may be a stimulus generator for applying electrical stimuli to any branch of the 8^{th} nerve in a generally constant rate independent of audio modulation, such that it is perceived as active silence. See e.g., U.S. Pat. No. 6,175,767. The neurostimulator may be a subcranially implanted electromechanical system that has an input transducer and an output stimulator that converts a mechanical sound vibration into an electrical signal. See e.g., U.S. Pat. No. 6,235,056. The neurostimulator may be a cochlear implant that has a rechargeable battery housed within the implant for storing and providing electrical power. See e.g., U.S. Pat. No. 6,067,474. Other neurostimulators that are used as cochlear implants are described in, e.g., U.S. Pat. Nos. 6,358,281; 6,308,101 and 5,603,726.

Cochlear implants, which may benefit from having the subject polymer composition infiltrated into adjacent tissue according to the present invention, include commercially available products. Several commercially available devices are available for the treatment of patients with significant sensorineural hearing loss and are suitable for use with the present invention. For example, the HIRESOLUTION Bionic Ear System (Boston Scientific Corp., Nattick, Mass.) consists of the HIRES AURIA Processor which processes sound and sends a digital signal to the HIRES 90K Implant that has been surgically implanted in the inner ear. See e.g., U.S. Pat. Nos. 6,636,768; 6,309,410 and 6,259,951. The electrode array that transmits the impulses generated by the HIRES 90K Implant to the nerve may benefit from having the subject polymer composition infiltrated into tissue adjacent to the electrode-nerve interface. The PULSARci cochlear implant (MED-EL GMBH, Innsbruck, Austria, see e.g., U.S. Pat. Nos. 6,556,870 and 6,231,604) and the NUCLEUS 3 cochlear implant system (Cochlear Corp., Lane Cove, Australia, see e.g., U.S. Pat. Nos. 6,807,445; 6,788,790; 6,554,762; 6,537,200 and 6,394,947) are other commercial examples of cochlear implants whose electrodes may benefit from having the subject polymer composition infiltrated into tissue adjacent to the electrode-nerve interface.

Regardless of the specific design features, for cochlear implants to be effective in sensorineural deafness, the electrode arrays must be accurately positioned adjacent to the afferent auditory nerve fibers. If excessive scar tissue growth or extracellular matrix deposition occurs around the leads, efficacy can be compromised. Cochlear implants having the subject polymer compositions infiltrated into tissue adjacent to the electrode-tissue interface can increase the efficiency of impulse transmission and increase the duration that these devices function clinically. Cochlear implants may also benefit from release of a therapeutic agent able to prevent or inhibit infection in the vicinity of the implant site. In one aspect, the device includes cochlear implants and/or leads having the subject polymer composition comprising an anti-scarring agent and/or anti-infective agent infiltrated into tissue adjacent to where the cochlear implant and/or leads are or will be implanted. In another aspect, the present invention provides leads having the subject polymer composition comprising an anti-scarring agent and/or anti-infective agent infiltrated into tissue adjacent to the cochlear tissue surrounding the lead.

In another aspect, the present invention provides cochlear implants having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with cochlear implants have been described above.

Polymeric compositions may be infiltrated around implanted cochlear implants by applying the composition directly and/or indirectly into and/or onto (a) tissue adjacent to the cochlear implant; (b) the vicinity of the cochlear implant-tissue interface; (c) the region around the cochlear implant; and (d) tissue surrounding the cochlear implant. Methods for infiltrating the subject polymer compositions into tissue adjacent to a cochlear implant include delivering the polymer composition: (a) to the surface of the cochlear implant (e.g., as an injectable, paste, gel or mesh) during the implantation procedure; (b) to the surface of the tissue (e.g., as an injectable, paste, gel, in situ forming gel or mesh) immediately prior to, or during, implantation of the cochlear implant; (c) to the surface of the cochlear implant and/or the tissue surrounding the implanted cochlear implant (e.g., as an injectable, paste, gel, in situ forming gel or mesh) immediately after the implantation of the cochlear implant; (d) by topical application of the composition into the anatomical space where the cochlear implant may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the cochlear implant as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device, including the device only, lead only, electrode only and/or a combination thereof.

According to one aspect, any fibrosis-inhibiting and/or anti-infective agent described above may be utilized in the practice of the present invention. In one aspect of the invention, the subject polymer compositions infiltrated into tissue adjacent to cochlear implants may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As cochlear implants are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges-from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm²to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

(8) Electrical Stimulation to Promote Bone Growth

In one aspect, the subject polymer compositions may be infiltrated into tissue adjacent to an electrical bone stimulation device. The subject polymer compositions may contain a therapeutic agent (e.g., an anti-scarring and/or anti-infective agent).

Electrical stimulation can also be used to stimulate bone growth. For example, the stimulation device may be an electrode and generator having a strain response piezoelectric material which responds to strain by generating a charge to enhance the anchoring of an implanted bone prosthesis to the natural bone. See e.g., U.S. Pat. No. 6,143,035. If excessive scar tissue growth or extracellular matrix deposition occurs around the leads, efficacy can be compromised. Electrical bone stimulation devices having the subject polymer compositions infiltrated into tissue adjacent to the electrode-tissue interface can increase the efficiency of impulse transmission and increase the duration that these devices function clinically. Electrical bone stimulation devices may also benefit from release of a therapeutic agent able to prevent or inhibit infection in the vicinity of the implant site. In one aspect, the device includes electrical bone stimulation devices and/or leads having the subject polymer composition comprising an anti-scarring agent and/or anti-infective agent infiltrated into tissue adjacent to where the electrical bone stimulation device and/or leads are or will be implanted. In another aspect, the present invention provides leads having the subject polymer composition comprising an anti-scarring agent and/or anti-infective agent infiltrated into tissue adjacent to the bone tissue surrounding the electrical lead.

In another aspect, the present invention provides electrical bone stimulation devices having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with electrical bone stimulation devices have been described above.

Polymeric compositions may be infiltrated around implanted electrical bone stimulation devices by applying the composition directly and/or indirectly into and/or onto (a) tissue adjacent to the electrical bone stimulation device; (b) the vicinity of the electrical bone stimulation device-tissue interface; (c) the region around the electrical bone stimulation device; and (d) tissue surrounding the electrical bone stimulation device. Methods for infiltrating the subject polymer compositions into tissue adjacent to an electrical bone stimulation device include delivering the polymer composition: (a) to the surface of the electrical bone stimulation device (e.g., as an injectable, paste, gel or mesh) during the implantation procedure; (b) to the surface of the tissue (e.g., as an injectable, paste, gel, in situ forming gel or mesh) immediately prior to, or during, implantation of the electrical bone stimulation device; (c) to the surface of the electrical bone stimulation device and/or the tissue surrounding the implanted electrical bone stimulation device (e.g., as an injectable, paste, gel, in situ forming gel or mesh) immediately after the implantation of the electrical bone stimulation device; (d) by topical application of the composition into the anatomical space where the electrical bone stimulation device may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the electrical bone stimulation device as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device, including the device only, lead only, electrode only and/or a combination thereof.

According to one aspect, any fibrosis-inhibiting and/or anti-infective agent described above may be utilized in the practice of the present invention. In one aspect of the invention, the subject polymer compositions infiltrated into tissue adjacent to electrical bone stimulation devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As electrical bone stimulation devices are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

Although numerous neurostimulation devices have been described above, all possess similar design features and cause similar unwanted tissue reactions following implantation and may introduce or promote infection in the area of the implant site. It should be obvious to one of skill in the art that commercial neurostimulation devices not specifically sited above as well as next-generation and/or subsequently-developed commercial neurostimulation products are to be anticipated and are suitable for use under the present invention. The neurostimulation device, particularly the lead(s), must be positioned in a very precise manner to ensure that stimulation is delivered to the correct anatomical location in the nervous system. All, or parts, of a neurostimulation device can migrate following surgery, or excessive scar (or glial) tissue growth can occur around the implant, which can lead to a reduction in the performance of these devices. Neurostimulator devices having the subject polymer compositions infiltrated into tissue adjacent to the electrode-tissue interface can be used to increase the efficacy and/or the duration of activity of the implant (particularly for fully-implanted, battery-powered devices). Neurstimulator devices may also benefit from release of a therapeutic agent able to prevent or inhibit infection in the vicinity of the implant site. In one aspect, the present invention provides neurostimulator devices having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with neurostimulator devices have been described above. These compositions can further include one or more fibrosis-inhibiting agents such that the overgrowth of granulation, fibrous, or gliotic tissue is inhibited or reduced and/or one or more anti-infective agents such that infection in the vicinity of the implant site is inhibited or prevented.

Cardiac Rhythm Management (CRM) Devices

In one aspect, the subject polymer compositions may be infiltrated into tissue adjacent to a cardiac rhythm management device. The subject polymer compositions may contain a therapeutic agent (e.g., an anti-scarring and/or anti-infective agent).

The medical device may also be a cardiac pacemaker device where a pulse generator delivers an electrical impulse to myocardial tissue (often specialized conduction fibres) via an implanted lead in order to regulate cardiac rhythm. Typically, electrical leads are composed of a connector assembly, a lead body (i.e., conductor) and an electrode. Electrical leads may be unipolar, in which they are adapted to provide effective therapy with only one electrode. Multi-polar leads are also available, including bipolar, tripolar and quadripolar leads. Electrical leads may also have insulating sheaths which may include polyurethane or silicone-rubber coatings. Representative examples of electrical leads include, without limitation, medical leads, cardiac leads, pacer leads, pacing leads, pacemaker leads, endocardial leads, endocardial pacing leads, cardioversion/defibrillator leads, cardioversion leads, epicardial leads, epicardial defibrillator leads, patch defibrillators, patch leads, electrical patch, transvenous leads, active fixation leads, passive fixation leads and sensing leads Representative examples of CRM devices that utilize electrical leads include: pacemakers, LVAD's, defibrillators, implantable sensors and other electrical cardiac stimulation devices.

There are numerous pacemaker devices where the occurrence of a fibrotic reaction will adversely affect the functioning of the device or cause damage to the myocardial tissue. Typically, fibrotic encapsulation of the pacemaker lead (or the growth of fibrous tissue between the lead and the target myocardial tissue) slows, impairs, or interrupts electrical transmission of the impulse from the device to the myocardium. For example, fibrosis is often found at the electrode-myocardial interfaces in the heart, which may be attributed to electrical injury from focal points on the electrical lead. The fibrotic injury may extend into the tricuspid valve, which may lead to perforation. Fibrosis may lead to thrombosis of the subclavian vein; a condition which may be life-threatening. Electrical leads having the subject polymer compositions infiltrated into tissue adjacent to the electrode-tissue interface may help prolong the clinical performance of these devices. Not only can fibrosis cause the device to function suboptimally or not at all, it can cause excessive drain on battery life as increased energy is required to overcome the electrical resistance imposed by the intervening scar tissue. Similarly, fibrotic encapsulation of the sensing components of a rate-responsive pacemaker (described below) can impair the ability of the pacemaker to identify and correct rhythm abnormalities leading to inappropriate pacing of the heart or the failure to function correctly when required. Cardiac pacemaker devices and/or electrical leads may also benefit from release of a therapeutic agent able to prevent or inhibit infection in the vicinity of the implant site.

Several different electrical pacing devices are used in the treatment of various cardiac rhythm abnormalities including pacemakers, implantable cardioverter defibrillators (ICD), left ventricular assist devices (LVAD), and vagus nerve stimulators (stimulates the fibers of the vagus nerve which in turn innervate the heart). The pulse generating portion of device sends electrical impulses via implanted leads to the muscle (myocardium) or conduction tissue of the heart to affect cardiac rhythm or contraction. Pacing can be directed to one or more chambers of the heart. Cardiac pacemakers may be used to block, mask, or stimulate electrical signals in the heart to treat dysfunctions, including, without limitation, atrial rhythm abnormalities, conduction abnormalities and ventricular rhythm abnormalities. ICDs are used to depolarize the ventricals and re-establish rhythm if a ventricular arrhythmia occurs (such as asystole or ventricular tachycardia) and LVADs are used to assist ventricular contraction in a failing heart.

Representative examples of patents which describe pacemakers and pacemaker leads include U.S. Pat. Nos. 4,662,382, 4,782,836, 4,856,521, 4,860,751, 5,101,824, 5,261,419, 5,284,491, 6,055,454, 6,370,434, and 6,370,434. Representative examples of electrical leads include those found on a variety of cardiac devices, such as cardiac stimulators (see e.g., U.S. Pat. Nos. 6,584,351 and 6,115,633), pacemakers (see e.g., U.S. Pat. Nos. 6,564,099; 6,246,909 and 5,876,423), implantable cardioverter-defibrillators (ICDs), other defibrillator devices (see e.g., U.S. Pat. No. 6,327,499), defibrillator or demand pacer catheters (see e.g., U.S. Pat. No. 5,476,502) and Left Ventricular Assist Devices (see e.g., U.S. Pat. No. 5,503,615).

Cardiac rhythm devices, and in particular the lead(s) that deliver the electrical pulsation, must be positioned in a very precise manner to ensure that stimulation is delivered to the correct anatomical location in the heart. All, or parts, of a pacing device can migrate following surgery, or excessive scar tissue growth can occur around the lead, which can lead to a reduction in the performance of these devices (as described previously). Cardiac rhythm management devices having the subject polymer compositions infiltrated into tissue adjacent to the electrode-tissue interface can be used to increase the efficacy and/or the duration of activity (particularly for fully-implanted, battery-powered devices) of the implant. Cardiac rhythm management devices may also benefit from release of a therapeutic agent able to prevent or inhibit infection in the vicinity of the implant site. In one aspect, the present invention provides cardiac rhythm management devices and/or leads having the subject polymer composition comprising an anti-scarring agent and/or anti-infective agent infiltrated into tissue adjacent to where the cardiac rhythm management device and/or leads are or will be implanted. In another aspect, the present invention provides leads having the subject polymer composition comprising an anti-scarring agent and/or anti-infective agent infiltrated into tissue adjacent to the tissue where the lead will be implanted.

In another aspect, the present invention provides cardiac rhythm management devices having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with cardiac rhythm management devices have been described above.

Polymeric compositions may be infiltrated around implanted cardiac rhythm management devices by applying the composition directly and/or indirectly into and/or onto (a) tissue adjacent to the cardiac rhythm management device; (b) the vicinity of the cardiac rhythm management device-tissue interface; (c) the region around the cardiac rhythm management device; and (d) tissue surrounding the cardiac rhythm management device. Methods for infiltrating the subject polymer compositions into tissue adjacent to a cardiac rhythm management device include delivering the polymer composition: (a) to the surface of the cardiac rhythm management device (e.g., as an injectable, paste, gel or mesh) during the implantation procedure; (b) to the surface of the tissue (e.g., as an injectable, paste, gel, in situ forming gel or mesh) immediately prior to, or during, implantation of the cardiac rhythm management device; (c) to the surface of the cardiac rhythm management device and/or the tissue surrounding the implanted cardiac rhythm management device (e.g., as an injectable, paste, gel, in situ forming gel or mesh) immediately after the implantation of the cardiac rhythm management device; (d) by topical application of the composition into the anatomical space where the cardiac rhythm management device may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the cardiac rhythm management device as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device, including the device only, lead only, electrode only and/or a combination thereof.

According to one aspect, any fibrosis-inhibiting and/or anti-infective agent described above may be utilized in the practice of the present invention. In one aspect of the invention, the subject polymer compositions infiltrated into tissue adjacent to cardiac rhythm management devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As cardiac rhythm management devices are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 pg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

For greater clarity, several specific cardiac rhythm management devices and treatments will be described in greater detail below.

(1) Cardiac Pacemakers

In one aspect, the subject polymer compositions may be infiltrated into tissue adjacent to a cardiac pacemaker. The subject polymer compositions may contain a therapeutic agent (e.g., an anti-scarring and/or anti-infective agent).

Cardiac rhythm abnormalities are extremely common in clinical practice and the incidence increases in frequency with both age and the presence of underlying coronary artery disease or myocardial infarction. A litany of arrythmias exists, but they are generally categorized into conditions where the heart beats too slowly (bradyarrythmias—such heart block, sinus node dysfunction) or too quickly (tachyarrhythmias—such as atrial fibrillation, WPW syndrome, ventricular fibrillation). A pacemaker functions by sending an electrical pulse (a pacing pulse) that travels via an electrical lead to the electrode (at the tip of the lead) which delivers an electrical impulse to the heart that initiates a heartbeat. The leads and electrodes can be located in one chamber (either the right atrium or the right ventricle—called single-chamber pacemakers) or there can be electrodes in both the right atrium and the right ventricle (called dual-chamber pacemakers). Electrical leads may be implanted on the exterior of the heart (e.g., epicardial leads) by a surgical procedure, or they can be connected to the endocardial surface of the heart via a catheter, guidewire or stylet. In some pacemakers, the device assumes the rhythm generating function of the heart and fires at a regular rate. In other pacemakers, the device merely augments the heart's own pacing function and acts “on demand” to provide pacing assistance as required (called “adaptive-rate” pacemakers); the pacemaker receives feedback on heart rhythm (and hence when to fire) from an electrode sensor located on the lead. Other pacemakers, called rate responsive pacemakers, have special sensors that detect changes in body activity (such as movement of the arms and legs, respiratory rate) and adjust pacing up or down accordingly.

Numerous pacemakers and pacemaker leads are suitable for use in this invention. For example, the pacing lead may have an increased resistance to fracture by being composed of an elongated coiled conductor mounted within a lumen of a lead body whereby it may be coupled electrically to a stranded conductor. See e.g., U.S. Pat. Nos. 6,061,598 and 6,018,683. The pacing lead may have a coiled conductor with an insulated sheath, which has a resistance to crush fatigue in the region between the rib and clavicle. See e.g., U.S. Pat. No. 5,800,496. The pacing lead may be expandable from a first, shorter configuration to a second, longer configuration by being composed of slideable inner and outer overlapping tubes containing a conductor. See e.g., U.S. Pat. No. 5,897,585. The pacing lead may have the means for temporarily making the first portion of the lead body stiffer by using a magnet-rheologic fluid in a cavity that stiffens when exposed to a magnetic field. See e.g., U.S. Pat. No. 5,800,497. The pacing lead may be a coil configuration composed of a plurality of wires or wire bundles made from a duplex titanium alloy. See e.g., U.S. Pat. No. 5,423,881. The pacing lead may be composed of a wire wound in a coil configuration with the wire composed of stainless steel having a composition of at least 22% nickel and 2% molybdenum. See e.g., U.S. Pat. No. 5,433,744. Other pacing leads are described in, e.g., U.S. Pat. Nos. 6,489,562; 6,289,251 and 5,957,967.

In another aspect, the electrical lead used in the practice of this invention may have an active fixation element for attachment to tissue. For example, the electrical lead may have a rigid fixation helix with microgrooves that are dimensioned to minimize the foreign body response following implantation. See e.g., U.S. Pat. No. 6,078,840. The electrical lead may have an electrode/anchoring portion with a dual tapered self-propelling spiral electrode for attachment to vessel wall. See e.g., U.S. Pat. No. 5,871,531. The electrical lead may have a rigid insulative electrode head carrying a helical electrode. See e.g., U.S. Pat. No. 6,038,463. The electrical lead may have an improved anchoring sleeve designed with an introducer sheath to minimize the flow of blood through the sheath during introduction. See e.g., U.S. Pat. No. 5,827,296. The electrical lead may be composed of an insulated electrical conductive portion and a lead-in securing section having a longitudinally rigid helical member which may be screwed into tissue. See e.g., U.S. Pat. No. 4,000,745.

Suitable leads for use in the practice of this invention also include multi-polar leads with multiple electrodes connected to the lead body. For example, the electrical lead may be a multi-electrode lead whereby the lead has two internal conductors and three electrodes with two electrodes coupled by a capacitor integral with the lead. See e.g., U.S. Pat. No. 5,824,029. The electrical lead may be a lead body with two straight sections and a bent third section with associated conductors and electrodes whereby the electrodes are bipolar. See e.g., U.S. Pat. No. 5,995,876. In another aspect, the electrical lead may be implanted by using a catheter, guidewire or stylet. For example, the electrical lead may be composed of an elongated insulative lead body having a lumen with a conductor mounted within the lead body and a resilient seal having an expandable portion through which a guidewire may pass. See e.g., U.S. Pat. No. 6,192,280.

Cardiac pacemakers, which may benefit from having the subject polymer composition infiltrated into adjacent tissue according to the present invention, include commercially available products. Commercially available pacemakers suitable for the practice of the invention include the KAPPA SR 400 Series single-chamber rate-responsive pacemaker system, the KAPPA DR 400 Series dual-chamber rate-responsive pacemaker system, the KAPPA 900 and 700 Series single-chamber rate-responsive pacemaker system, and the KAPPA 900 and 700 Series dual-chamber rate-responsive pacemaker system by Medtronic, Inc. Medtronic pacemaker systems utilize a variety leads including the CAPSURE Z Novus, CAPSUREFIX Novus, CAPSUREFIX, CAPSURE SP Novus, CAPSURE SP, CAPSURE EPI and the CAPSURE VDD which may benefit from having the subject polymer composition infiltrated into adjacent tissue. Pacemaker systems and associated leads that are made by Medtronic are described in, e.g., U.S. Pat. Nos. 6,741,893; 5,480,441; 5,411,545; 5,324,310; 5,265,602; 5,265,601; 5,241,957 and 5,222,506. Medtronic also makes a variety of steroid-eluting leads including those described in, e.g., U.S. Pat. Nos. 5,987,746; 6,363,287; 5,800,470; 5,489,294; 5,282,844 and 5,092,332. The INSIGNIA single-chamber and dual-chamber system, PULSAR MAX II DR dual-chamber adaptive-rate pacemaker, PULSAR MAX II SR single-chamber adaptive-rate pacemaker, DISCOVERY II DR dual-chamber adaptive-rate pacemaker, DISCOVERY II SR single-chamber adaptive-rate pacemaker, DISCOVERY II DDD dual-chamber pacemaker, and the DISCOVERY II SSI dingle-chamber pacemaker systems made by Guidant Corp. (Indianapolis, Ind.) are also suitable pacemaker systems for the practice of this invention. Once again, the leads from the Guidant pacemaker systems may benefit from having the subject polymer composition infiltrated into adjacent tissue. Pacemaker systems and associated leads that are made by Guidant are described in, e.g., U.S. Pat. Nos. 6,473,648; 6,345,204; 6,321,122; 6,152,954; 5,769,881; 5,284,136; 5,086,773 and 5,036,849. The AFFINITY DR, AFFINITY VDR, AFFINITY SR, AFFINITY DC, ENTITY, IDENTITY, IDENTITY ADX, INTEGRITY, INTEGRITY μDR, INTEGRITY ADx, MICRONY, REGENCY, TRILOGY, and VERITY ADx, pacemaker systems and leads from St. Jude Medical, Inc. (St. Paul, Minn.) may also be suitable for use with the present invention to improve electrical transmission and sensing by the pacemaker leads. Pacemaker systems and associated leads that are made by St. Jude Medical are described in, e.g., U.S. Pat. Nos. 6,763,266; 6,760,619; 6,535,762; 6,246,909; 6,198,973; 6,183,305; 5,800,468 and 5,716,390. Alternatively, the fibrosis-inhibiting agent may be infiltrated into the region around the electrode-cardiac muscle interface under the present invention. It should be obvious to one of skill in the art that commercial pacemakers not specifically sited as well as next-generation and/or subsequently developed commercial pacemaker products are to be anticipated and are suitable for use under the present invention.

Regardless of the specific design features, for pacemakers to be effective in the management of cardiac rhythm disorders, the leads must be accurately positioned adjacent to the targeted cardiac muscle tissue. If excessive scar tissue growth or extracellular matrix deposition occurs around the leads, efficacy can be compromised. Pacemaker leads having the subject polymer compositions infiltrated into tissue adjacent to the electrode-tissue and/or sensor-tissue interface, can increase the efficiency of impulse transmission and rhythm sensing, thereby increasing efficacy and battery longevity. Pacemaker leads may also benefit from release of a therapeutic agent able to prevent or inhibit infection in the vicinity of the implant site. Cardiac pacemakers and/or leads having the subject polymer composition comprising an anti-scarring agent and/or anti-infective agent infiltrated into tissue adjacent to where the cardiac pacemaker and/or leads are or will be implanted. In another aspect, the present invention provides pacemaker leads having the subject polymer composition comprising an anti-scarring agent and/or anti-infective agent infiltrated into tissue adjacent to the myocardial tissue where the lead will be implanted.

In another aspect, the present invention provides cardiac pacemakers having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with cardiac pacemakers have been described above.

Polymeric compositions may be infiltrated around implanted cardiac pacemakers by applying the composition directly and/or indirectly into and/or onto (a) tissue adjacent to the cardiac pacemaker; (b) the vicinity of the cardiac pacemaker-tissue interface; (c) the region around the cardiac pacemaker; and (d) tissue surrounding the cardiac pacemaker. Methods for infiltrating the subject polymer compositions into tissue adjacent to a cardiac pacemaker include delivering the polymer composition: (a) to the surface of the cardiac pacemaker (e.g., as an injectable, paste, gel or mesh) during the implantation procedure; (b) to the surface of the tissue (e.g., as an injectable, paste, gel, in situ forming gel or mesh) immediately prior to, or during, implantation of the cardiac pacemaker; (c) to the surface of the cardiac pacemaker and/or the tissue surrounding the implanted cardiac pacemaker (e.g., as an injectable, paste, gel, in situ forming gel or mesh) immediately after the implantation of the cardiac pacemaker; (d) by topical application of the composition into the anatomical space where the cardiac pacemaker may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the cardiac pacemaker as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device, including the device only, lead only, electrode only and/or a combination thereof.

According to one aspect, any fibrosis-inhibiting and/or anti-infective agent described above may be utilized in the practice of the present invention. In one aspect of the invention, the subject polymer compositions infiltrated into tissue adjacent to cardiac pacemakers may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As cardiac pacemakers are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

(2) Implantable Cardioverter Defibrillator (ICD) Systems

In one aspect, the subject polymer compositions may be infiltrated into tissue adjacent to an implantable cardioverter defibrillator (ICD) system. The subject polymer compositions may contain a therapeutic agent (e.g., an anti-scarring and/or anti-infective agent).

Implantable cardioverter defibrillator (ICD) systems are similar to pacemakers (and many include a pacemaker system), but are used for the treatment of tachyarrhythmias such as ventricular tachycardia or ventricular fibrillation. An ICD consists of a mini-computer powered by a battery which is connected to a capacitor to helps the ICD charge and store enough energy to deliver therapy when needed. The ICD uses sensors to monitor the activity of the heart and the computer analysizes the data to determine when and if an arrhythmia is present. An ICD lead, which is inserted via a vein (called “transvenous” leads; in some systems the lead is implanted surgically—called an epicardial lead—and sewn onto the surface of the heart), connects into the pacing/computer unit. The lead, which is usually placed in the right ventricle, consists of an insulated wire and an electrode tip that contains a sensing component (to detect cardiac rhythm) and a shocking coil. A single-chamber ICD has one lead placed in the ventricle which defibrillates and paces the ventricle, while a dual-chamber ICD defibrillates the ventricle and paces the atrium and the ventricle. In some cases, an additional lead is required and is placed under the skin next to the rib cage or on the surface of the heart. In patients who require tachyarrhythmia management of the ventricle and atrium, a second coil is placed in the atrium to treat atrial tachycardia, atrial fibrillation and other arrhythmias. If a tachyarrhythmia is detected, a pulse is generated and propagated via the lead to the shocking coil which delivers a charge sufficient to depolarize the muscle and cardiovert or defibrillate the heart.

Several ICD systems have been described and are suitable for use in the practice of this invention. Representative examples of ICD's and associated components are described in U.S. Pat. Nos. 3,614,954, 3,614,955, 4,375,817, 5,314,430, 5,405,363, 5,607,385, 5,697,953, 5,776,165, 6,067,471, 6,169,923, and 6,152,955. Several ICD leads are suitable for use in the practice of this invention. For example, the defibrillator lead may be a linear assembly of sensors and coils formed into a loop which includes a conductor system for coupling the loop system to a pulse generator. See e.g., U.S. Pat. No. 5,897,586. The defibrillator lead may have an elongated lead body with an elongated electrode extending from the lead body, such that insulative tubular sheaths are slideably mounted around the electrode. See e.g., U.S. Pat. No. 5,919,222. The defibrillator lead may be a temporary lead with a mounting pad and a temporarily attached conductor with an insulative sleeve whereby a plurality of wire electrodes are mounted. See e.g., U.S. Pat. No. 5,849,033. Other defibrillator leads are described in, e.g., U.S. Pat. No. 6,052,625. In another aspect, the electrical lead may be adapted to be used for pacing, defibrillating or both applications. For example, the electrical lead may be an electrically insulated, elongated, lead body sheath enclosing a plurality of lead conductors that are separated from contacting one another. See e.g., U.S. Pat. No. 6,434,430. The electrical lead may be composed of an inner lumen adapted to receive a stiffening member (e.g., guide wire) that delivers fluoro-visible media. See e.g., U.S. Pat. No. 6,567,704. The electrical lead may be a catheter composed of an elongated, flexible, electrically nonconductive probe contained within an electrically conductive pathway that transmits electrical signals, including a defibrillation pulse and a pacer pulse, depending on the need that is sensed by a governing element. See e.g., U.S. Pat. No. 5,476,502. The electrical lead may have a low electrical resistance and good mechanical resistance to cyclical stresses by being composed of a conductive wire core formed into a helical coil covered by a layer of electrically conductive material and an electrically insulating sheath covering. See e.g., U.S. Pat. No. 5,330,521. Other electrical leads that may be adapted for use in pacing and/or defibrillating applications are described in, e.g., U.S. Pat. Nos. 6,556,873.

ICDs, which may benefit from having the subject polymer composition infiltrated into adjacent tissue according to the present invention, include commercially available products. Commercially available ICDs suitable for the practice of the invention include the GEM III DR dual-chamber ICD, GEM III VR ICD, GEM II ICD, GEM ICD, GEM III AT atrial and ventricular arrhythmia ICD, JEWEL AF dual-chamber ICD, MICRO JEWEL ICD, MICRO JEWEL II ICD, JEWEL Plus ICD, JEWEL ICD, JEWEL ACTIVE CAN ICD, JEWEL PLUS ACTIVE CAN ICD, MAXIMO DR ICD, MAXIMO VR ICD, MARQUIS DR ICD, MARQUIS VR system, and the INTRINSIC dual-chamber ICD by Medtronic, Inc. Medtronic ICD systems utilize a variety leads including the SPRINT FIDELIS, SPRINT QUATRO SECURE steroid-eluting bipolar lead, Subcutaneous Lead System Model 6996SQ subcutaneous lead, TRANSVENE 6937A transvenous lead, and the 6492 Unipolar Atrial Pacing Lead which may benefit from having the subject polymer composition infiltrated into adjacent tissue. ICD systems and associated leads that are made by Medtronic are described in, e.g., U.S. Pat. Nos. 6,038,472; 5,849,031; 5,439,484; 5,314,430; 5,165,403; 5,099,838 and 4,708,145. The VITALITY 2 DR dual-chamber ICD, VITALITY 2 VR single-chamber ICD, VITALITY AVT dual-chamber ICD, VITALITY DS dual-chamber ICD, VITALITY DS VR single-chamber ICD, VITALITY EL dual-chamber ICD, VENTAK PRIZM 2 DR dual-chamber ICD, and VENTAK PRIZM 2 VR single-chamber ICD systems made by Guidant Corp. are also suitable ICD systems for the practice of this invention. Once again, the leads from the Guidant ICD systems may benefit from having the subject polymer composition infiltrated into adjacent tissue. Guidant sells the FLEXTEND Bipolar Leads, EASYTRAK Lead System, FINELINE Leads, and ENDOTAK RELIANCE ICD Leads. ICD systems and associated leads that are made by Guidant are described in, e.g., U.S. Pat. Nos. 6,574,505; 6,018,681; 5,697,954; 5,620,451; 5,433,729; 5,350,404; 5,342,407; 5,304,139 and 5,282,837. Biotronik, Inc. (Germany) sells the POLYROX Endocardial Leads, KENTROX SL Quadripolar ICD Leads, AROX Bipolar Leads, and MAPOX Bipolar Epicardial Leads (see e.g., U.S. Pat. Nos. 6,449,506; 6,421,567; 6,418,348; 6,236,893 and 5,632,770). The CONTOUR MD ICD, PHOTON μ DR ICD, PHOTON μ VR ICD, ATLAS+ HF ICD, EPIC HF ICD, EPIC+ HF ICD systems and leads from St. Jude Medical may also benefit from having the subject polymer composition infiltrated into adjacent tissue to improve electrical transmission and sensing by the ICD leads (see e.g., U.S. Pat. Nos. 5,944,746; 5,722,994; 5,662,697; 5,542,173; 5,456,706 and 5,330,523). Alternatively, the fibrosis-inhibiting agent may be infiltrated into the region around the electrode-cardiac muscle interface under the present invention. It should be obvious to one of skill in the art that commercial ICDs not specifically sited as well as next-generation and/or subsequently developed commercial ICD products are to be anticipated and are suitable for use under the present invention.

Regardless of the specific design features, for ICDs to be effective in the management of cardiac rhythm disorders, the leads must be accurately positioned adjacent to the targeted cardiac muscle tissue. If excessive scar tissue growth or extracellular matrix deposition occurs around the leads, efficacy can be compromised. ICD leads having the subject polymer compositions infiltrated into tissue adjacent to the electrode-tissue and/or sensor-tissue interface, can increase the efficiency of impulse transmission and rhythm sensing, thereby increasing efficacy, preventing inappropriate cardioversion, and improving battery longevity. ICDs may also benefit from release of a therapeutic agent able to prevent or inhibit infection in the vicinity of the implant site. In one aspect, the device includes ICDs and/or leads having the subject polymer composition comprising an anti-scarring agent and/or anti-infective agent infiltrated into tissue adjacent to where the ICD and/or leads are or will be implanted. In another aspect, the present invention provides ICD leads having the subject polymer composition comprising an anti-scarring agent and/or anti-infective agent infiltrated into tissue adjacent to the myocardial tissue surrounding the lead.

In another aspect, the present invention provides ICDs having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with ICDs have been described above.

Polymeric compositions may be infiltrated around implanted ICDs by applying the composition directly and/or indirectly into and/or onto (a) tissue adjacent to the ICD; (b) the vicinity of the ICD-tissue interface; (c) the region around the ICD; and (d) tissue surrounding the ICD. Methods for infiltrating the subject polymer compositions into tissue adjacent to a ICD include delivering the polymer composition: (a) to the surface of the ICD (e.g., as an injectable, paste, gel or mesh) during the implantation procedure; (b) to the surface of the tissue (e.g., as an injectable, paste, gel, in situ forming gel or mesh) immediately prior to, or during, implantation of the ICD; (c) to the surface of the ICD and/or the tissue surrounding the implanted ICD (e.g., as an injectable, paste, gel, in situ forming gel or mesh) immediately after the implantation of the ICD; (d) by topical application of the composition into the anatomical space where the ICD may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the ICD as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device, including the device only, lead only, electrode only and/or a combination thereof.

According to one aspect, any fibrosis-inhibiting and/or anti-infective agent described above may be utilized in the practice of the present invention. In one aspect of the invention, the subject polymer compositions infiltrated into tissue adjacent to ICDs may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As ICDs are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

(3) Vagus Nerve Stimulation for the Treatment of Arrhythmia

In one aspect, the subject polymer compositions may be infiltrated into tissue adjacent to a vagal nerve stimulation (VNS) device. The subject polymer compositions may contain a therapeutic agent (e.g., an anti-scarring and/or anti-infective agent).

A neurostimulation device may also be used to stimulate the vagus nerve and affect the rhythm of the heart. Since the vagus nerve provides innervation to the heart, including the conduction system (including the SA node), stimulation of the vagus nerve may be used to treat conditions such as supraventricular arrhythmias, angina pectoris, atrial tachycardia, atrial flutter, atrial fibrillation and other arrhythmias that result in low cardiac output.

As described above, in VNS a bipolar electrical lead is surgically implanted such that it transmits electrical stimulation from the pulse generator to the left vagus nerve in the neck. The pulse generator is an implanted, lithium carbon monofluoride battery-powered device that delivers a precise pattern of stimulation to the vagus nerve. The pulse generator can be programmed (using a programming wand) by the cardiologist to treat a specific arrhythmia.

Products such as these have been described, for example, in U.S. Pat. Nos. 6,597,953 and 6,615,085. For example, the neurostimulator may be a vagal-stimulation apparatus which generates pulses at a frequency that varies automatically based on the excitation rates of the vagus nerve. See e.g., U.S. Pat. Nos. 5,916,239 and 5,690,681. The neurostimulator may be an apparatus that detects characteristics of tachycardia based on an electrogram and delivers a preset electrical stimulation to the nervous system to depress the heart rate. See e.g., U.S. Pat. No. 5,330,507. The neurostimulator may be an implantable heart stimulation system composed of two sensors, one for atrial signals and one for ventricular signals, and a pulse generator and control unit, to ensure sympatho-vagal stimulation balance. See e.g., U.S. Pat. No. 6,477,418. The neurostimulator may be a device that applies electrical pulses to the vagus nerve at a programmable frequency that is adjusted to maintain a lower heart rate. See e.g., U.S. Pat. No. 6,473,644. The neurostimulator may provide electrical stimulation to the vagus nerve to induce changes to electroencephalogram readings as a treatment for epilepsy, while controlling the operation of the heart within normal parameters. See e.g., U.S. Pat. No. 6,587,727.

VNS devices, which may benefit from having the subject polymer composition infiltrated into adjacent tissue according to the present invention, include commercially available products. A commercial example of a VNS system is the product produced by Cyberonics Inc. that consists of the Model 300 and Model 302 leads, the Model 101 and Model 102R pulse generators, the Model 201 programming wand and Model 250 programming software, and the Model 220 magnets. These products manufactured by Cyberonics, Inc. may be described, for example, in U.S. Pat. Nos. 5,928,272; 5,540,730 and 5,299,569.

Regardless of the specific design features, for vagal nerve stimulation to be effective in arrhythmias, the leads must be accurately positioned adjacent to the left vagus nerve. If excessive scar tissue growth or extracellular matrix deposition occurs around the VNS leads, this can reduce the efficacy of the device. VNS devices having the subject polymer compositions infiltrated into tissue adjacent to the electrode-tissue interface can increase the efficiency of impulse transmission and increase the duration that these devices function clinically. VNS devices may also benefit from release of a therapeutic agent able to prevent or inhibit infection in the vicinity of the implant site. In one aspect, the device includes VNS devices and/or leads having the subject polymer composition comprising an anti-scarring agent and/or anti-infective agent infiltrated into tissue adjacent to where the VNS device and/or leads are or will be implanted. In another aspect, the present invention provides leads having the subject polymer composition comprising an anti-scarring agent and/or anti-infective agent infiltrated into tissue adjacent to the vagus nerve where the lead will be implanted.

In another aspect, the present invention provides VNS devices having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with VNS devices have been described above.

Polymeric compositions may be infiltrated around implanted VNS devices by applying the composition directly and/or indirectly into and/or onto (a) tissue adjacent to the VNS device; (b) the vicinity of the VNS device-tissue interface; (c) the region around the VNS device; and (d) tissue surrounding the VNS device. Methods for infiltrating the subject polymer compositions into tissue adjacent to a VNS device include delivering the polymer composition: (a) to the surface of the VNS device (e.g., as an injectable, paste, gel or mesh) during the implantation procedure; (b) to the surface of the tissue (e.g., as an injectable, paste, gel, in situ forming gel or mesh) immediately prior to, or during, implantation of the VNS device; (c) to the surface of the VNS device and/or the tissue surrounding the implanted VNS device (e.g., as an injectable, paste, gel, in situ forming gel or mesh) immediately after the implantation of the VNS device; (d) by topical application of the composition into the anatomical space where the VNS device may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the VNS device as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device, including the device only, lead only, electrode only and/or a combination thereof.

According to one aspect, any fibrosis-inhibiting and/or anti-infective agent described above may be utilized in the practice of the present invention. In one aspect of the invention, the subject polymer compositions infiltrated into tissue adjacent to VNS devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As VNS devices are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

Although numerous cardiac rhythm management (CRM) devices have been described above, all possess similar design features and cause similar unwanted fibrous tissue reactions following implantation and may introduce or promote infection in the area of the implant site. It should be obvious to one of skill in the art that commercial CRM devices not specifically sited above as well as next-generation and/or subsequently-developed commercial CRM products are to be anticipated and are suitable for use under the present invention. The CRM device, particularly the lead(s), must be positioned in a very precise manner to ensure that stimulation is delivered to the correct anatomical location within the atrium and/or ventricle. All, or parts, of a CRM device can migrate following surgery, or excessive scar tissue growth can occur around the implant, which can lead to a reduction in the performance of these devices. CRM devices having the subject polymer compositions infiltrated into tissue adjacent to the electrode-tissue interface can be used to increase the efficacy and/or the duration of activity of the implant (particularly for fully-implanted, battery-powered devices). CRM devices may also benefit from release of a therapeutic agent able to prevent or inhibit infection in the vicinity of the implant site. In one aspect, the present invention provides CRM devices having the subject polymer compositions infiltrated into adjacent tissue, where the subject polymer compositions may include a therapeutic agent (e.g., an anti-scarring and/or anti-infective agent). These compositions can further include one or more fibrosis-inhibiting agents such that the overgrowth of granulation fibrous, or gliotic tissue is inhibited or reduced and/or one or more anti-infective agents such that infection in the vicinity of the implant site is inhibited or prevented.

Implantable Sensors

In one aspect, the subject polymer compositions may be infiltrated into tissue adjacent to an implantable sensor. The subject polymer compositions may contain a therapeutic agent (e.g., an anti-scarring and/or anti-infective agent).

Implantable sensors are provided that can be used to detect physiological levels or changes in the body. There are numerous sensor devices where the occurrence of a fibrotic reaction will adversely affect the functioning of the device or the biological problem for which the device was implanted or used. Proper clinical functioning of an implanted sensor is dependent upon intimate anatomical contact with the target tissues and/or body fluids. Scarring around the implanted device may degrade the electrical components and characteristics of the device-tissue interface, and the device may fail to function properly. The formation of scar tissue between the sensing device and the adjacent (target) tissue can prevent the flow of physical, chemical and/or biological information (e.g., fluid levels, drug levels, metabolite levels, glucose levels, pressure etc.) from reaching the detection mechanism of the sensor. Similarly if a “foreign body” response occurs and causes the implanted sensor to become encapsulated by scar (i.e., the body “walls off” the sensor with fibrous tissue), the sensor will receive biological information that is not reflective of the organism as a whole. If the sensor is detecting conditions inside the capsule (i.e., levels detected in a microenvironment), and these conditions are not consistent with those outside the capsule (i.e., within the body as a whole—the microenvironment), it will record information that is not representative of systemic levels. Implantation of an implantable sensor may also introduce or promote infection in the vicinity of the implant site.

Sensors or transducers may be located deep within the body for monitoring a variety of physiological properties, such as temperature, pressure, strain, fluid flow, metabolite levels (e.g., electrolytes, glucose), drug levels, chemical properties, electrical properties, magnetic properties, and the like. Representative examples of implantable sensors for use in the practice of the invention include, blood and tissue glucose monitors, electrolyte sensors, blood constituent sensors, temperature sensors, pH sensors, optical sensors, amperometric sensors, pressure sensors, biosensors, sensing transponders, strain sensors, activity sensors and magnetoresistive sensors.

Numerous types of implantable sensors and transducers have been described. For example, the implantable sensor may be a micro-electronic device that is implanted around the large bowels to control bowel function by detecting rectal contents and stimulating peristaltic contractions to empty the bowels when it is convenient. See, e.g., U.S. Pat. No. 6,658,297. The implantable sensor may be used to measure pH in the GI tract. A representative example of such a pH sensing device is the BRAVO pH Monitoring System from Medtronic, Inc. (Minneapolis, Minn.). The implantable sensor may be part of a GI catheter or probe that includes a sensor portion connected to an electrical or optical measurement device and a sensitive polymeric material that undergoes an irreversible change when exposed to cumulative action of an external medium. See, e.g., U.S. Pat. No. 6,006,121. The implantable sensor may be a component of a central venous catheter (CVC) (e.g., a jugular vein catheter) system. For example, the device may be composed of a catheter body having at least one oxygen sensor and a distal heat exchange region in which the catheter body is formed with coolant supply and return lumens to provide heat exchange within a body to prevent overheating due to severe brain trauma or ischemia due to stroke. See, e.g., U.S. Pat. No. 6,652,565. A CVC may include a thermal mass and a temperature sensor to measure blood temperature. See, e.g., U.S. Pat. No. 6,383,144.

In one aspect, the present invention provides implantable sensors having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with implantable sensors have been described above.

Polymeric compositions may be infiltrated around implanted implantable sensors by applying the composition directly and/or indirectly into and/or onto (a) tissue adjacent to the implantable sensor; (b) the vicinity of the implantable sensor-tissue interface; (c) the region around the implantable sensor; and (d) tissue surrounding the implantable sensor. Methods for infiltrating the subject polymer compositions into tissue adjacent to an implantable sensor include delivering the polymer composition: (a) to the surface of the implantable sensor (e.g., as an injectable, paste, gel or mesh) during the implantation procedure; (b) to the surface of the tissue (e.g., as an injectable, paste, gel, in situ forming gel or mesh) immediately prior to, or during, implantation of the implantable sensor; (c) to the surface of the implantable sensor and/or the tissue surrounding the implanted implantable sensor (e.g., as an injectable, paste, gel, in situ forming gel or mesh) immediately after the implantation of the implantable sensor; (d) by topical application of the composition into the anatomical space where the implantable sensor may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the implantable sensor as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device, including the device only, sensor only, detector only and/or a combination thereof.

According to one aspect, any fibrosis-inhibiting and/or anti-infective agent described above may be utilized in the practice of the present invention. In one aspect of the invention, the subject polymer compositions infiltrated into tissue adjacent to implantable sensors may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As implantable sensors are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

Several specific implantable sensor devices and treatments will be described in greater detail below.

(1) Blood and Glucose Monitors

In one aspect, the subject polymer compositions may be infiltrated into tissue adjacent to a glucose monitor. The subject polymer compositions may contain a therapeutic agent (e.g., an anti-scarring and/or anti-infective agent).

Glucose monitors are used to detect changes in blood glucose, specifically for the management and treatment of patients with diabetes mellitus. Diabetes is a metabolic disorder of glucose metabolism that afflicts tens of millions of people in the developed countries of the world. This disease is characterized by the inability of the body to properly utilize and metabolize carbohydrates, particularly glucose. Normally, the finely-tuned balance between glucose in the blood and glucose in the bodily tissue cells is maintained by insulin, a hormone produced by the pancreas. If the pancreas becomes defective and insulin is produced in inadequate amounts to reduce blood glucose levels (Type I diabetes), or if the body becomes insensitive to the glucose-lowering effects of insulin despite adequate pancreatic insulin production (Type II diabetes), the result is diabetes. Accurate detection of blood glucose levels is essential to the management of diabetic patients because the dosage and timing of administration of insulin and/or other hypoglycemic agents are titrated depending upon changes in glucose levels in response to the medication. If the dosage is too high, blood glucose levels drop too low, resulting in confusion and potentially even loss of consciousness. If the dosage is too low, blood glucose levels rise too high, leading to excessive thirst, urination, and changes in metabolism known as ketoacidosis. If the timing of medication administration is incorrect, blood glucose levels can fluctuate wildly between the two extremes—a situation that is thought to contribute to some of the long-term complications of diabetes such as heart disease, kidney failure and blindness. Since in the extreme, all these conditions can be life threatening, careful and continuous monitoring of glucose levels is a critical aspect of diabetes management. One way to detect changes in glucose levels and to continuously sense when levels of glucose become too high or too low in diabetes patients is to implant a glucose monitor. As the glucose monitor detects changes in the blood glucose levels, insulin can be administered by external injection or via an implantable insulin pump to maintain blood glucose levels within an acceptable physiologic range.

Numerous types of blood and tissue glucose monitors are suitable for use in the practice of the invention. For example, the glucose monitor may be delivered to the vascular system transluminally using a catheter on a stent platform. See, e.g., U.S. Pat. No. 6,442,413. The glucose monitor may be composed of glucose sensitive living cells that monitor blood glucose levels and produce a detectable electrical or optical signal in response to changes in glucose concentrations. See, e.g., U.S. Pat. Nos. 5,101,814 and 5,190,041. The glucose monitor may be a small diameter flexible electrode implanted subcutaneously which may be composed of an analyte-responsive enzyme designed to be an electrochemical glucose monitor. See, e.g., U.S. Pat. Nos. 6,121,009 and 6,514,718. The implantable sensor may be a closed loop insulin delivery system whereby there is a sensing means that detects the patient's blood glucose level based on electrical signals and then stimulates either an insulin pump or the pancreas to supply insulin. See, e.g., U.S. Pat. Nos. 6,558,345 and 6,093,167. Other glucose monitors are described in, for e.g., U.S. Pat. Nos. 6,579,498; 6,565,509 and 5,165,407. Minimally invasive glucose monitors include the GLUCOWATCH G2 BIOGRAPHER from Cygnus Inc. (see cygn.com); see, e.g., U.S. Pat. Nos. 6,546,269; 6,687,522; 6,595,919 and U.S. patent application Nos. 20040062759A1; 20030195403A1; and 20020091312A1.

Glucose monitors, which may benefit from having the subject polymer composition infiltrated into adjacent tissue according to the present invention, include commercially available products. Numerous commercially available blood and tissue glucose monitoring devices are suitable for the practice of this invention. Although virtually any implantable glucose monitor may be utilized, several specific commercial and development stage examples are described below for greater clarity.

The CONTINUOUS GLUCOSE MONITORING SYSTEM (CGMS) from Medtronic MiniMed, Inc. (Northridge, Calif.; see minimed.com); see, e.g., U.S. Pat. Nos. 6,520,326; 6,424,847; 6,360,888; 5,605,152; 6,804,544; and U.S. patent application No. 20040167464A1. The CGMS system is surgically implanted in the subcutaneous tissue of the abdomen and stores tissue glucose readings every 5 minutes. Infiltrating the subject polymer composition into tissue adjacent to the sensor may prolong the activity of this device because it often must be removed after several days (approximately 3), in part because it loses its sensitivity as a result of the local tissue reaction to the device.

The CONTINUOUS GLUCOSE MONITORING DEVICE from TheraSense (Alameda, Calif., see therasense.com) which utilizes a disposable, miniaturized electrochemical sensor that is inserted under the patient's skin using a spring-loaded insertion device. The sensor measures glucose levels in the interstitial fluid every five minutes, with the ability to store results for future analysis. See, e.g., US20040186365A1; US20040106858A1 and US200301761 83A1. Even though the device can store up to a month of data and has alarms for high and low glucose levels, it must be replaced every few days because it loses its accuracy as a result of the foreign body reaction to the implant. Infiltrating the subject polymer composition into tissue adjacent to this sensor may prolong its activity, enhance its performance and reduce the frequency of replacement. Another electrochemical sensor that may benefit from the present invention is the multilayered implantable electrochemical sensor from Isense (Portland, Oreg.). This system consists of a semipermeable membrane, a catalytic membrane which generates an electrical current in the presence of glucose, and a specificity membrane to reduce interference from other substances.

The SMSI glucose sensor (Sensors for Medicine and Sciences, Inc., Montgomery County, Maryland; see s4ms.com) is designed to be implanted under the skin in a short outpatient procedure. The sensor is designed to automatically measure interstitial glucose every few minutes, without any user intervention. The sensor implant communicates wirelessly with a small external reader, allowing the user to monitor glucose levels continuously or on demand. The reader is designed to be able to track the rate of change of glucose levels and warn the user of impending hypo- or hyperglycemia. The operational life of the sensor implant is about 6-12 months, after which it may be replaced.

Animas Corporation (West Chester, Pa.; animascorp.com) is developing an implantable glucose sensor that measures the near-infrared absorption of blood based on spectroscopy or optical sensing placed around a vein. The Animas glucose monitor may be tied to an insulin infusion pump to provide a closed-loop control of blood glucose levels. Scar tissue over the sensor distorts the ability of the device to correctly gather optical information and the sensor may thus benefit from the present invention.

DexCom, Inc. (San Diego, Calif.; see dexcom.com) is developing their Continuous Glucose Monitoring System which is an implantable sensor that wirelessly transmits continuous blood glucose readings to an external receiver. The receiver displays the current glucose value every 30 seconds, as well as one-hour, three-hour and nine-hours trended values, and sounds an alert when a high or low glucose excursion is detected. This device features an implantable sensor that is placed in the subcutaneous tissue and continuously monitors tissue (interstitial fluid) glucose levels for both type 1 and type 2 diabetics. This device may also include a unique microarchitectural arrangement in the sensor region that allows accurate data to be obtained over long periods of time. Glucose monitoring devices and associated systems that are developed by DexCom, Inc. are described in, for example, U.S. Pat. Nos. 6,741,877; 6,702,857 and 6,558,321. Unfortunately, even though the battery and circuitry of monitoring devices allows long-term functioning, a foreign body response and/or encapsulation of the implant affect the ability of the device to detect glucose levels accurately for prolonged periods in a percentage of implants. Infiltrating the subject polymer composition into tissue adjacent to this device may allow it to accurately detect glucose levels for longer periods of time after implantation, reduce the number of devices that fail and decrease the incidence of replacement.

Also of particular interest in the practice of this invention is glucose monitoring systems that utilize a glucose-responsive polymer as part of their detection mechanism. M-Biotech (Salt Lake City, Utah) is developing a continuous monitoring system that consists of subcutaneous implantation of a glucose-responsive hydrogel combined with a pressure transducer. See, e.g., U.S. Patent Nos.; and. The hydrogel responds to changes in glucose concentration by either shrinking or swelling and the expansion or contraction is detected by the pressure transducer. The transducer converts the information into an electrical signal and sends a wireless signal to a display device. Cybersensors (Berkshire, UK) produces a capsule-like sensor implanted under the skin and an external receiver/transmitter that captures the data and powers the capsule via RF signals (see, e.g., GB 2335496 and U.S. Pat. No. 6,579,498) Issued by the UK Patent and Trademark Office). The sensor capsule is composed of a glucose affinity polymer and contains a physical sensor and an RF microchip; the entire capsule is further enclosed in a semipermeable membrane. The glucose affinity polymer exhibits rheological changes when exposed to glucose (in the range of 3-15 nM) by becoming thinner and less viscous as glucose concentrations increase. This reversible reaction can be detected by the physical sensor and converted into a signal. These aforementioned systems are suitable for infiltrating the subject polymer composition into tissue adjacent to the implanted sensor as provided in the present invention.

Another glucose sensing device is under development by Advanced Biosensors (Mentor, Ohio) that consists of small (150 μm wide by 2 mm long), biocompatible, silicon-based needles that are implanted under the skin. The device senses glucose levels in the dermis and transmits data wirelessly. Unfortunately, a foreign body response and/or encapsulation of the implant affect the ability of the device to detect glucose levels accurately for longer than 7 days. Infiltrating the subject polymer composition into tissue adjacent to this device may allow it to accurately detect glucose levels for longer periods of time and extend the effective lifespan of the device.

Regardless of the specific design features of implantable blood, tissue, or interstitial fluid glucose monitoring devices, for accurate detection of physical, chemical and/or physiological properties, the device must be accurately positioned adjacent to the tissue. In particular, the detector of the sensing mechanism must be exposed to glucose levels that are identical to (or representative of) those found in the bloodstream. If excessive scar tissue growth or extracellular matrix deposition occurs around the device, this can impair the movement of glucose from the tissue to the detector and render it ineffective. Similarly if a “foreign body” response occurs and causes the implanted glucose sensor to become encapsulated by fibrous tissue, the sensor will be detecting glucose levels in the capsule. If glucose levels inside the capsule are not consistent with those outside the capsule (i.e., within the body as a whole), it will record information that is not representative of systemic levels. This can cause the physician or the patient to administer the wrong dosage of hypoglycemic drugs (such as insulin) with potentially serious consequences. Blood, tissue or interstitial fluid glucose monitoring devices having the subject polymer compositions infiltrated into tissue adjacent to the implant can reduce scarring and/or encapsulation of the implant and increase the efficiency and accuracy of glucose detection, minimize insulin dosing errors, assist in the maintenance of correct blood glucose levels, increase the duration that these devices function clinically, and/or reduce the frequency of implant replacement. Glucose monitoring devices such as these may also benefit from release of a therapeutic agent able to prevent or inhibit infection in the vicinity of the implant site. In one aspect, the device includes blood, tissue and interstitial fluid glucose monitoring devices having the subject polymer composition comprising an anti-scarring agent and/or anti-infective agent infiltrated into tissue adjacent to where the device is or will be implanted. In another aspect, the present invention provides glucose monitoring devices having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with glucose monitoring devices have been described above.

Polymeric compositions may be infiltrated around implanted glucose monitoring devices by applying the composition directly and/or indirectly into and/or onto (a) tissue adjacent to the glucose monitoring device; (b) the vicinity of the glucose monitoring device-tissue interface; (c) the region around the glucose monitoring device; and (d) tissue surrounding the glucose monitoring device. Methods for infiltrating the subject polymer compositions into tissue adjacent to a glucose monitoring device include delivering the polymer composition: (a) to the surface of the glucose monitoring device (e.g., as an injectable, paste, gel or mesh) during the implantation procedure; (b) to the surface of the tissue (e.g., as an injectable, paste, gel, in situ forming gel or mesh) immediately prior to, or during, implantation of the glucose monitoring device; (c) to the surface of the glucose monitoring device and/or the tissue surrounding the implanted glucose monitoring device (e.g., as an injectable, paste, gel, in situ forming gel or mesh) immediately after the implantation of the glucose monitoring device; (d) by topical application of the composition into the anatomical space where the glucose monitoring device may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the glucose monitoring device as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device, including the device only, sensor only, detector only and/or a combination thereof.

According to one aspect, any fibrosis-inhibiting and/or anti-infective agent described above may be utilized in the practice of the present invention. In one aspect of the invention, the subject polymer compositions infiltrated into tissue adjacent to glucose monitoring devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As glucose monitoring devices are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

(2) Pressure and Stress Sensors

In one aspect, the subject polymer compositions may be infiltrated into tissue adjacent to a pressure and/or stress sensor. The subject polymer compositions may contain a therapeutic agent (e.g., an anti-scarring and/or anti-infective agent).

Pressure or stress monitors may be used to detect increasing pressure or stress within the body. Implantable pressure transducers and sensors are used for temporary or chronic use in a body organ, tissue or vessel for recording absolute pressure. Many different designs and operating systems have been proposed and placed into temporary or chronic use for patients with a variety of medical conditions. Indwelling pressure sensors for temporary use of a few days or weeks are available, however, chronically or permanently implantable pressure sensors have also been used. Pressure sensors may detect many types of bodily pressures, such as, but not limited to blood pressure and fluid flow, pressure within aneurysm sacs, intracranial pressure, and mechanical pressure associated with bone fractures.

Numerous types of pressure monitors are suitable for use in the practice of the invention. For example, the implantable sensor may detect body fluid absolute pressure at a selected site and ambient operating temperature by using a lead, sensor module, sensor circuit (including electrical conductors) and means for providing voltage. See, e.g., U.S. Pat. No. 5,535,752. The implantable sensor may be an intracranial pressure monitor that provides an analogue data signal which is converted electronically to a digital pulse. See, e.g., U.S. Pat. No. 6,533,733. The implantable sensor may be a barometric pressure sensor enclosed in an air chamber which is used for deriving reference pressure data for use in combination with an implantable medical device, such as a pacemaker. See, e.g., U.S. Pat. No. 6,152,885. The implantable sensor may be adapted to be inserted into a body passageway to monitor a parameter related to fluid flow through an endoluminal implant (e.g., stent). See, e.g., U.S. Pat. No. 5,967,986. The implantable sensor may be a passive sensor with an inductor-capacitor circuit having a resonant frequency which is adapted for the skull of a patient to sense intracranial pressure. See, e.g., U.S. Pat. No. 6,113,553. The implantable sensor may be a self-powered strain sensing system that generates a strain signal in response to stresses that may be produced at a bone fixation device. See, e.g., U.S. Pat. No. 6,034,296. The implantable sensor may be a component of a perfusion catheter. The catheter may include a wire electrode and a lumen for perfusing saline around the wire, which is designed for measuring a potential difference across the GI wall and for simultaneous measurement of pressure. See, e.g., U.S. Pat. No. 5,551,425. The implantable sensor may be part of a CNS device; for example, an intracranial pressure sensor which is mounted within the skull of a body at the situs where the pressure is to be monitored and a means of transmitting the pressure externally from the skull. See, e.g., U.S. Pat. No. 4,003,141. The implantable sensor may be a component of a left ventricular assist device. For example, the VAD may be a blood pump adapted to be joined in flow communication between the left ventricle and the aorta using an inlet flow pressure sensor and a controller that may adjust speed of pump based on sensor feedback. See, e.g., U.S. Pat. No. 6,623,420.

Pressure and/or stress sensor devices, which may benefit from having the subject polymer composition infiltrated into adjacent tissue according to the present invention, include commercially available products. Numerous commercially available and experimental pressure and stress sensor devices are suitable for the practice of the invention. By way of illustration, a selection of these devices and implants are described in the following paragraphs.

A device from CardioMEMS (Atlanta, Ga.; @cardiomems.com, a partnership between the Georgia Institute of Technology and the Cleveland Clinic) which can be inserted into an aneurysm sac to monitor pressure within the sac and thereby alert a medical specialist to the filing of the sac with fluid, possibly to rupture-provoking levels. Endovascular aneurysm repair (EVAR) is often performed using a stent graft which isolates the aneurysm from the circulation. However, persistent leakage of blood into the aneurysm sac results in ongoing pressure build-up in the sac and a resultant risk of rupture. The CardioMEMS device is implanted into the aneurysm sac after EVAR to monitor pressure in the isolated sac in order to detect which patients are at increasing risk of rupture. The pressure sensor features an inductive-capacitive resonant circuit with a variable capacitor. Since capacitance varies with the pressure in the environment in which the capacitor is placed, it can detect changes in local pressure. Data is generated by using external excitation systems that induce an oscillating current in the sensor and detecting the frequency of oscillation (which is then used to calculate pressure). Unfortunately, even though the circuitry allows long-term functioning, a foreign body response and/or encapsulation of the implant affect the ability of the device to detect accurate pressure levels in the aneurysm (i.e., the device detects the pressure in the microenvironment of the capsule, not of the aneurysm sac as a whole). Implantation of a sensor may also introduce or promote infection in the vicinity of the implant site. Infiltrating the subject polymer composition into tissue adjacent to this device may allow it to accurately detect pressure levels for longer periods of time after implantation and reduce the number of devices that fail.

MicroStrain Inc. (Williston, Vt., @microstrain.com) has developed a family of wireless implantable sensors for measuring strain, position and motion within the body. These sensors can measure, for example, eye tremor, depth of corneal implant, orientation sensor for improved tooth crown prep, mayer ligament strains, spinal ligament strains, vertebral bone strains, elbow ligament strains, emg and ekg data, 3DM-G for measurement of orientation and motion, wrist ligament strains, hip replacement sensors for measuring micromotion, implant subsidence, knee ligament strain, ankle ligament strain, Achilles tendon strain, foot arch support strains, force within foot insoles. The company provides a knee prosthesis that can measure in vivo compressive forces and transmit the data in real time. Patents describing this technology, and components used in the manufacture of devices for this technology include U.S. Pat. Nos. 6,714,763; 6,625,517; 6,622,567; 6,588,282; 6,529,127; 6,499,368; 6,433,629; 5,887,351; 5,777,467; 5,497,147; and 4,993,428. U.S. patent applications describing this technology, and components used in the manufacture of devices for this technology include 20040113790; 20040078662; 20030204361; 20030158699; 20030047002; 20020190785; 20020170193; 20020088110; 20020085174; 20010054317; and 20010033187.

Mesotec (Hannover, Germany; @mesotec.com), in collaboration with several German institutes (e.g., Fraunhofer Institute of Microelectronic Circuits and Systems), has developed an implantable intraocular pressure sensor system, called the MESOGRAPH, which can continuously monitor intraocular pressure. This is desirable, e.g., in order to identify the onset of glaucoma. The CMOS-based sensor can be implanted during standard surgical procedures and is inductively linked to an external unit integrated into a spectacle frame. The glasses are in turn linked via a cable to a portable data logger. Data is relayed upstream to the glasses using a modulated RF carrier operating at 13.56 MHz and a switchable load, while power comes downstream to the sensor. By varying the diameter of the polysilicon diaphragms in the on-chip micromechanical vacuum gap capacitors, the pressure range to which the sensor responds can be adapted between 50 kNm-2 and 3.5 MNm-2. The device consists of a fine, foldable coil for telemetric coupling and a very small miniaturized pressure sensor. The sensor is manufactured on a micro-technological basis and serves for continuous, long-term reading and monitoring of intraocular pressure. Chip and coil are integrated in modified soft intraocular lenses, which can be implanted in the patient's eye during today's common surgical procedures. Unfortunately, the device often fails after initially successful implantation because a foreign body response and/or encapsulation of the implant affect the ability of it to detect accurate pressure levels in the eye (i.e., the device detects the pressure in the microenvironment of the capsule surrounding the implant, not intraocular pressure as a whole). Implantation of a sensor may also introduce or promote infection in the vicinity of the implant site. Infiltrating the subject polymer composition into the eye tissue adjacent to this device may allow it to accurately detect pressure levels for longer periods of time after implantation and reduce the number of devices that fail.

Regardless of the specific design features of the pressure and/or stress sensor, for accurate detection of physical and/or physiological properties (such as pressure), the device must be accurately positioned within the tissue and receive information that is representative of conditions as a whole. If excessive scar tissue growth or extracellular matrix deposition occurs around the device, the sensor may receive erroneous information that compromises its efficacy or the scar tissue may block the flow of biological information to the sensor. For example, many devices fail after initially successful implantation because encapsulation of the implant causes it to detect nonrelevant pressure levels (i.e., the device detects the pressure in the microenvironment of the capsule surrounding the implant, not the pressure of the larger environment) Pressure and stress sensing devices having the subject polymer compositions infiltrated into tissue adjacent to the implant can increase the efficiency of detection and increase the duration that these devices function clinically. Pressure and stress sensing devices such as these may also benefit from release of a therapeutic agent able to prevent or inhibit infection in the vicinity of the implant site. In one aspect, the device includes implantable sensor devices having the subject polymer composition comprising an anti-scarring agent and/or anti-infective agent infiltrated into tissue adjacent to where the device is or will be implanted. In another aspect, the present invention provides pressure or stress sensing devices having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with pressure or stress sensing devices have been described above.

Polymeric compositions may be infiltrated around implanted pressure or stress sensing devices by applying the composition directly and/or indirectly into and/or onto (a) tissue adjacent to the pressure or stress sensing device; (b) the vicinity of the pressure or stress sensing device-tissue interface; (c) the region around the pressure or stress sensing device; and (d) tissue surrounding the pressure or stress sensing device. Methods for infiltrating the subject polymer compositions into tissue adjacent to a pressure or stress sensing device include delivering the polymer composition: (a) to the surface of the pressure or stress sensing device (e.g., as an injectable, paste, gel or mesh) during the implantation procedure; (b) to the surface of the tissue (e.g., as an injectable, paste, gel, in situ forming gel or mesh) immediately prior to, or during, implantation of the pressure or stress sensing device; (c) to the surface of the pressure or stress sensing device and/or the tissue surrounding the implanted pressure or stress sensing device (e.g., as an injectable, paste, gel, in situ forming gel or mesh) immediately after the implantation of the pressure or stress sensing device; (d) by topical application of the composition into the anatomical space where the pressure or stress sensing device may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the pressure or stress sensing device as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device, including the device only, sensor only, detector only and/or a combination thereof.

According to one aspect, any fibrosis-inhibiting and/or anti-infective agent described above may be utilized in the practice of the present invention. In one aspect of the invention, the subject polymer compositions infiltrated into tissue adjacent to pressure or stress sensing devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As pressure or stress sensing devices are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

(3) Cardiac Sensors

In one aspect, the subject polymer compositions may be infiltrated into tissue adjacent to a cardiac sensor device. The subject polymer compositions may contain a therapeutic agent (e.g., an anti-scarring and/or anti-infective agent).

In another aspect, the implantable sensor may be a device configured to detect properties in the heart or in cardiac muscle tissue. Cardiac sensors are used to detect parameters associated with the performance of the heart as monitored at any given time point along a prolonged time period. Typically, monitoring of the heart is often conducted to detect changes associated with heart disease, such as chronic heart failure (CHF). By monitoring patterns associated with heart function, deterioration based on hemodynamic changes can be detected (parameters such as cardiac output, ejection fraction, pressure, ventricular wall motion, etc.). This constant direct monitoring is central to disease management in patients that present with CHF. By monitoring hemodynamic measures directly using implantable sensors, a hemodynamic crisis can be detected and the appropriate medications and interventions selected.

Numerous types of cardiac sensors are suitable for use in the practice of the invention. For example, the implantable sensor may be an activity sensor incorporating a magnet and a magnetoresistive sensor that provides a variable activity signal as part of a cardiac device. See, e.g., U.S. Pat. Nos. 6,430,440 and 6,411,849. The implantable sensor may monitor blood pressure in a heart chamber by emitting wireless communication to a remote device. See, e.g., U.S. Pat. No. 6,409,674. The implantable sensor may be an accelerometer-based cardiac wall motion sensor which transduces accelerations of cardiac tissue to a cardiac stimulation device by using electrical signals. See, e.g., U.S. Pat. No. 5,628,777. The implantable sensor may be implanted in the heart's cavity with an additional sensor implanted in a blood vessel to detect pressure and flow within heart's cavity. See, e.g., U.S. Pat. No. 6,277,078.

Cardiac sensors, which may benefit from having the subject polymer composition infiltrated into adjacent tissue according to the present invention, include commercially available products. Commercially available cardiac sensor devices suitable for the practice of the invention include Biotronik's (Biotronik GmbH & Co., Berlin, Germany, see biotronik.com) CARDIAC AIRBAG ICD SYSTEM is a rhythm monitoring device that offers rescue shock capability delivering 30 Joule shock therapies for up to 3 episodes of ventricular fibrillation. In addition to the rescue shock capability the system can also provide bradycardia pacing and VT monitoring. The PROTOS family of pacemakers from Biotronik (see biotronikusa.com) also incorporates pacing sensor capability called Closed Loop Simulation.

Blood flow and tissue perfusion monitors can be used to monitor noncardiac tissue as well. Researchers at Oak Ridge National Laboratory have developed a wireless sensor that monitors blood flow to a transplanted organ for the early detection of transplant rejection.

Medtronic (Minneapolis, Minn.; see medtronic.com) is developing their CHRONICLE implantable product, which is designed to continuously monitor a patient's intracardiac pressures, heart rate and physical activity using a sensor placed directly in the heart's chamber. The patient periodically downloads this information to a home-based device that transmits this physiologic data securely over the Internet to a physician.

Regardless of the specific design features of the cardiac sensor, for accurate detection of physical and/or physiological properties (such as pressure, flow rates, etc.), the device must be accurately positioned within the heart muscle, chambers or great vessels and receive information that is representative of conditions as a whole. If excessive scar tissue growth or extracellular matrix deposition occurs around the sensing device, the sensor may receive erroneous information that compromises its efficacy, or the scar tissue may block the flow of biological information to the detector mechanism of the sensor. For example, many cardiac sensors fail after initially successful implantation because encapsulation of the implant causes it to detect nonrelevant levels (i.e., the device detects conditions in the microenvironment of the capsule surrounding the implant, not the pressure of the larger environment). Cardiac sensor devices such as these may also benefit from release of a therapeutic agent able to prevent or inhibit infection in the vicinity of the implant site. Cardiac sensing devices having the subject polymer compositions infiltrated into tissue adjacent to the implant can increase the efficiency of detection and increase the duration that these devices function clinically. In one aspect, the device includes implantable sensor devices having the subject polymer composition comprising an anti-scarring agent and/or anti-infective agent infiltrated into tissue adjacent to where the device is or will be implanted. In another aspect, the present invention provides cardiac sensing devices having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with cardiac sensing devices have been described above.

Polymeric compositions may be infiltrated around implanted cardiac sensor devices by applying the composition directly and/or indirectly into and/or onto (a) tissue adjacent to the cardiac sensor device; (b) the vicinity of the cardiac sensor device-tissue interface; (c) the region around the cardiac sensor device; and (d) tissue surrounding the cardiac sensor device. Methods for infiltrating the subject polymer compositions into tissue adjacent to a cardiac sensor device include delivering the polymer composition: (a) to the surface of the cardiac sensor device (e.g., as an injectable, paste, gel or mesh) during the implantation procedure; (b) to the surface of the tissue (e.g., as an injectable, paste, gel, in situ forming gel or mesh) immediately prior to, or during, implantation of the cardiac sensor device; (c) to the surface of the cardiac sensor device and/or the tissue surrounding the implanted cardiac sensor device (e.g., as an injectable, paste, gel, in situ forming gel or mesh) immediately after the implantation of the cardiac sensor device; (d) by topical application of the composition into the anatomical space where the cardiac sensor device may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the cardiac sensor device as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device, including the device only, sensor only, detector only and/or a combination thereof.

According to one aspect, any fibrosis-inhibiting and/or anti-infective agent described above may be utilized in the practice of the present invention. In one aspect of the invention, the subject polymer compositions infiltrated into tissue adjacent to cardiac sensor devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As cardiac sensor devices are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

(4) Respiratory Sensors

In one aspect, the subject polymer compositions may be infiltrated into tissue adjacent to a respiratory sensor device. The subject polymer compositions may contain a therapeutic agent (e.g., an anti-scarring and/or anti-infective agent).

The implantable sensor may be a device configured to detect properties in the respiratory system. Respiratory sensors may be used to detect changes in breathing patterns. For example, a respiratory sensor may be used to detect sleep apnea, which is an airway disorder. There are two kinds of sleep apnea. In one condition, the body fails to automatically generate the neuromuscular stimulation necessary to initiate and control a respiratory cycle at the proper time. In the other condition, the muscles of the upper airway contract during the time of inspiration and thus the airway becomes obstructed. The cardiovascular consequences of apnea include disorders of cardiac rhythm (bradycardia, auriculoventricular block, ventricular extrasystoles) and hemodynamic disorders (pulmonary and systemic hypertension). This results in a stimulatory metabolic and mechanical effect on the autonomic nervous system and the potential to ultimately lead to increased morbidity. To treat this condition, implantable sensors may be used to monitor respiratory functioning to detect an apnea episode so the appropriate response (e.g., electrical stimulation to the nerves of the upper airway muscles) or other treatment can be provided.

Numerous types of respiratory sensors are suitable for use in the practice of the invention. For example, the implantable sensor may be a respiration element implanted in the thoracic cavity which is capable of generating a respiration signal as part of a ventilation system for providing gas to a host. See, e.g., U.S. Pat. No. 6,357,438. The implantable sensor may be composed of a sensing element connected to a lead body which is inserted into bone (e.g., manubrium) that communicates with the intrathoracic cavity to detect respiratory changes. See, e.g., U.S. Pat. No. 6,572,543.

Regardless of the specific design features of the respiratory sensor, for accurate detection of physical and/or physiological properties, the device must be accurately positioned adjacent to the tissue. If excessive scar tissue growth or extracellular matrix deposition occurs around the pulmonary function or airway sensing device, the sensor may receive erroneous information that compromises its efficacy, or the scar tissue may block the flow of biological information to the detector mechanism of the sensor. For example, many respiratory sensors (pulmonary function sensing devices) fail after initially successful implantation because encapsulation of the implant causes it to detect nonrelevant levels (i.e., the device detects conditions in the microenvironment of the capsule surrounding the implant, not the functioning of the respiratory system as whole). Respiratory sensor devices such as these may also benefit from release of a therapeutic agent able to prevent or inhibit infection in the vicinity of the implant site. Respiratory sensing devices having the subject polymer compositions infiltrated into tissue adjacent to the implant can increase the efficiency of detection and increase the duration that these devices function clinically. In one aspect, the device includes implantable sensor devices having the subject polymer composition comprising an anti-scarring agent and/or anti-infective agent infiltrated into tissue adjacent to where the device is or will be implanted. In another aspect, the present invention provides respiratory sensor devices having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with respiratory sensor devices have been described above.

Polymeric compositions may be infiltrated around implanted respiratory sensor devices by applying the composition directly and/or indirectly into and/or onto (a) tissue adjacent to the respiratory sensor device; (b) the vicinity of the respiratory sensor device-tissue interface; (c) the region around the respiratory sensor device; and (d) tissue surrounding the respiratory sensor device. Methods for infiltrating the subject polymer compositions into tissue adjacent to a respiratory sensor device include delivering the polymer composition: (a) to the surface of the respiratory sensor device (e.g., as an injectable, paste, gel or mesh) during the implantation procedure; (b) to the surface of the tissue (e.g., as an injectable, paste, gel, in situ forming gel or mesh) immediately prior to, or during, implantation of the respiratory sensor device; (c) to the surface of the respiratory sensor device and/or the tissue surrounding the implanted respiratory sensor device (e.g., as an injectable, paste, gel, in situ forming gel or mesh) immediately after the implantation of the respiratory sensor device; (d) by topical application of the composition into the anatomical space where the respiratory sensor device may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the respiratory sensor device as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device, including the device only, sensor only, detector only and/or a combination thereof.

According to one aspect, any fibrosis-inhibiting and/or anti-infective agent described above may be utilized in the practice of the present invention. In one aspect of the invention, the subject polymer compositions infiltrated into tissue adjacent to respiratory sensor devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As respiratory sensor devices are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

(5) Auditory Sensors

In one aspect, the subject polymer compositions may be infiltrated into tissue adjacent to an auditory sensor device. The subject polymer compositions may contain a therapeutic agent (e.g., an anti-scarring and/or anti-infective agent).

The implantable sensor may be a device configured to detect properties in the auditory system. Auditory sensors are used as part of implantable hearing systems for rehabilitation of pure sensorineural hearing losses, or combined conduction and inner ear hearing impairments. Hearing systems may include an implantable sensor which delivers an electrical signal which is processed by an implanted processor and delivered to an implantable electromechanical transducer which acts on the middle or inner ear. The auditory sensor acts as the microphone of the hearing system and acts to convert the incident airborne sound into an electrical signal.

Numerous types of auditory sensors as part of a hearing system are suitable for use in the practice of the invention. For example, the implantable sensor may generate an electrical audio signal as part of a hearing system for rehabilitation of hearing loss. See, e.g., U.S. Pat. No. 6,334,072. The implantable sensor may be a capacitive sensor which is mechanically or magnetically coupled to a vibrating auditory element, such as the malleus, which detects the time-varying capacitance values resulting from the vibrations. See, e.g., U.S. Pat. No. 6,190,306. The implantable sensor may be an electromagnetic sensor having a permanent magnet and a coil and a time-varying magnetic flux linkage based on the vibrations which are provided to an output stimulator for mechanical or electrical stimulation of the cochlea. See, e.g., U.S. Pat. No. 5,993,376.

Auditory sensors, which may benefit from having the subject polymer composition infiltrated into adjacent tissue according to the present invention, include commercially available products. Commercially available auditory sensor devices suitable for the practice of the invention include: the HIRES 90K Bionic Ear Implant, HIRESOLUTION SOUND, CLARION CII Bionic Ear, and CLARION 1.2, from Advanced Bionics (Sylmar, Calif., a Boston Scientific Company, see advancedbionics.com); see also U.S. Pat. Nos. 6,778,858; 6,754,537; 6,735,474; 6,731,986; 6,658,302; 6,636,768; 6,631,296; 6,628,991; 6,498,954; 6,487,453; 6,473,651; 6,415,187; and 6,415,185; the NUCLEUS 3 cochlear implant from Cochlear (Lane Cove NSW, Australia, see cochlear.com); see also U.S. Pat. Nos. 6,810,289; 6,807,455; 6,788,790; 6,782,619; 6,751,505; 6,736,770; 6,700,982; 6,697,674; 6,678,564; 6,620,093; 6,575,894; 6,570,363; 6,565,503; 6,554,762; 6,537,200; 6,525,512; 6,496,734; 6,480,820; 6,421,569; 6,411,855; 6,394,947; 6,392,386; 6,377,075; 6,301,505; 6,289,246; 6,116,413; 5,720,099; 5,653,742; 5,645,585; and U.S. patent application Publication Nos. 2004/0172102A1 and 2002/0138115A1; the PULSAR CI 100 and COMBI 40+ cochlear implants from Med-EI (Austria, see medel.com); see also U.S. patent application 20040039245A1, U.S. Pat. Nos. 6,600,955; 6,594,525; 6,556,870; and 5,983,139; the ALLHEAR implants from AllHear, Inc. (Aurora, Oreg.; see allhear.com); see also WO 01/50816; EP 1 245 134; and the DIGISONIC CONVEX, DIGISONIC AUDITORY BRAINSTEM, and DIGISONIC MULTI-ARRAY implants from MXM (France; see mxmiab.com); see also U.S. Pat. Nos. 5,123,422; EP 0 219 380; WO 04/002193; EP 1 244 400 A1; U.S. Pat. No. 6,428,484; US 20020095194A1; WO 01/50992.

Regardless of the specific design features of the auditory sensor, for accurate detection of sound, the device must be accurately positioned within the ear. If excessive scar tissue growth or extracellular matrix deposition occurs around the auditory sensor, the sensor may receive erroneous information that compromises its efficacy, or the scar tissue may block the flow of sound waves to the detector mechanism of the sensor. Auditory sensor devices such as these may also benefit from release of a therapeutic agent able to prevent or inhibit infection in the vicinity of the implant site. Auditory sensing devices having the subject polymer compositions infiltrated into tissue adjacent to the implant can increase the efficiency of sound detection and increase the duration that these devices function clinically. In one aspect, the device includes implantable sensor devices having the subject polymer composition comprising an anti-scarring agent and/or anti-infective agent infiltrated into tissue adjacent to where the device is or will be implanted. In another aspect, the present invention provides auditory sensor devices having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with auditory sensor devices have been described above.

Polymeric compositions may be infiltrated around implanted auditory sensor devices by applying the composition directly and/or indirectly into and/or onto (a) tissue adjacent to the auditory sensor device; (b) the vicinity of the auditory sensor device-tissue interface; (c) the region around the auditory sensor device; and (d) tissue surrounding the auditory sensor device. Methods for infiltrating the subject polymer compositions into tissue adjacent to an auditory sensor device include delivering the polymer composition: (a) to the surface of the auditory sensor device (e.g., as an injectable, paste, gel or mesh) during the implantation procedure; (b) to the surface of the tissue (e.g., as an injectable, paste, gel, in situ forming gel or mesh) immediately prior to, or during, implantation of the auditory sensor device; (c) to the surface of the auditory sensor device and/or the tissue surrounding the implanted auditory sensor device (e.g., as an injectable, paste, gel, in situ forming gel or mesh) immediately after the implantation of the auditory sensor device; (d) by topical application of the composition into the anatomical space where the auditory sensor device may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the auditory sensor device as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device, including the device only, sensor only, detector only and/or a combination thereof.

According to one aspect, any fibrosis-inhibiting and/or anti-infective agent described above may be utilized in the practice of the present invention. In one aspect of the invention, the subject polymer compositions infiltrated into tissue adjacent to auditory sensor devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As auditory sensor devices are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

(6) Electrolyte and Metabolite Sensors

In one aspect, the subject polymer compositions may be infiltrated into tissue adjacent to an electrolyte and/or metabolite sensor device. The subject polymer compositions may contain a therapeutic agent (e.g., an anti-scarring and/or anti-infective agent).

In another aspect, implantable sensors may be used to detect electrolytes and metabolites in the blood. For example, the implantable sensor may be a device to monitor constituent levels of metabolites or electrolytes in the blood by emitting a source of radiation directed towards blood such that it interacts with a plurality of detectors that provide an output signal. See, e.g., U.S. Pat. No. 6,122,536. The implantable sensor may be a biosensing transponder which is composed of a dye that has optical properties that change in response to changes in the environment, a photosensor to sense the optical changes, and a transponder for transmitting data to a remote reader. See, e.g., U.S. Pat. No. 5,833,603. The implantable sensor may be a monolithic bioelectronic device for detecting at least one analyte within the body of an animal. See, e.g., U.S. Pat. No. 6,673,596. Other sensors that measure chemical analytes are described in, e.g., U.S. Pat. Nos. 6,625,479 and 6,201,980.

If excessive scar tissue growth or extracellular matrix deposition occurs around the sensor, the sensor may receive erroneous information that compromises its efficacy, or the scar tissue may block the flow of metabolites or electrolytes to the detector mechanism of the sensor. For example, many metabolite/electrolyte sensing devices fail after initially successful implantation because encapsulation of the implant causes it to detect nonrelevant levels (i.e., the device detects conditions in the microenvironment of the capsule surrounding the implant, not blood levels). Sensing devices having the subject polymer compositions infiltrated into tissue adjacent to the implant can increase the efficiency of metabolite/electrolyte detection and increase the duration that these devices function clinically. Electrolyte and/or metabolite sensor device such as these may also benefit from release of a therapeutic agent able to prevent or inhibit infection in the vicinity of the implant site. In one aspect, the device includes implantable metabolite/electrolyte sensor devices having the subject polymer composition comprising an anti-scarring agent and/or anti-infective agent infiltrated into tissue adjacent to where the device is or will be implanted. In another aspect, the present invention provides metabolite/electrolyte sensor devices having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with metabolite/electrolyte sensor devices have been described above.

Polymeric compositions may be infiltrated around implanted metabolite/electrolyte sensor devices by applying the composition directly and/or indirectly into and/or onto (a) tissue adjacent to the metabolite/electrolyte sensor device; (b) the vicinity of the metabolite/electrolyte sensor device-tissue interface; (c) the region around the metabolite/electrolyte sensor device; and (d) tissue surrounding the metabolite/electrolyte sensor device. Methods for infiltrating the subject polymer compositions into tissue adjacent to a metabolite/electrolyte sensor device include delivering the polymer composition: (a) to the surface of the metabolite/electrolyte sensor device (e.g., as an injectable, paste, gel or mesh) during the implantation procedure; (b) to the surface of the tissue (e.g., as an injectable, paste, gel, in situ forming gel or mesh) immediately prior to, or during, implantation of the metabolite/electrolyte sensor device; (c) to the surface of the metabolite/electrolyte sensor device and/or the tissue surrounding the implanted metabolite/electrolyte sensor device (e.g., as an injectable, paste, gel, in situ forming gel or mesh) immediately after the implantation of the metabolite/electrolyte sensor device; (d) by topical application of the composition into the anatomical space where the metabolite/electrolyte sensor device may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the metabolite/electrolyte sensor device as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device, including the device only, sensor only, detector only and/or a combination thereof.

According to one aspect, any fibrosis-inhibiting and/or anti-infective agent described above may be utilized in the practice of the present invention. In one aspect of the invention, the subject polymer compositions infiltrated into tissue adjacent to metabolite/electrolyte sensor devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As metabolite/electrolyte sensor devices are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

Although numerous examples of implantable sensor devices have been described above, all possess similar design features and cause similar unwanted foreign body tissue reactions following implantation and may introduce or promote infection in the area of the implant site. It should be obvious to one of skill in the art that commercial sensor devices not specifically cited above as well as next-generation and/or subsequently-developed commercial sensor products are to be anticipated and are suitable for use under the present invention. The sensor device, particularly the sensing element, must be positioned in a very precise manner to ensure that detection is carried out at the correct anatomical location in the body. All, or parts, of a sensor device can migrate following surgery, or excessive scar tissue growth can occur around the implant, which can lead to a reduction in the performance of these devices. The formation of a fibrous capsule around the sensor can impede the flow of biological information to the detector and/or cause the device to detect levels that are not physiologically relevant (i.e., detect levels in the capsule instead of true physiological levels outside the capsule). Not only can this lead to incomplete or inaccurate readings, it can cause the physician or the patient to make incorrect therapeutic decisions based on the information generated. Implantable sensor devices having the subject polymer compositions infiltrated into tissue adjacent to the sensor-tissue interface can be used to increase the efficacy and/or the duration of activity of the implant. Implantable sensor devices may also benefit from release of a therapeutic agent able to prevent or inhibit infection in the vicinity of the implant site. In one aspect, the present invention provides implantable sensor devices having the subject polymer compositions infiltrated into adjacent tissue, where the subject polymer compositions may include a therapeutic agent (e.g., an anti-scarring and/or anti-infective agent). These compositions can further include one or more fibrosis-inhibiting agents such that the overgrowth of granulation, fibrous, or neointimal tissue is inhibited or reduced and/or one or more anti-infective agents such that infection in the vicinity of the implant site is inhibited or prevented.

Implantable Drug Delivery Devices and Pumps

In one aspect, the subject polymer compositions may be infiltrated into tissue adjacent to an implantable drug delivery device or pump. The subject polymer compositions may contain a therapeutic agent (e.g., an anti-scarring and/or anti-infective agent).

Implantable drug delivery devices and pumps are a means to provide prolonged, site-specific release of a therapeutic agent for the management of a variety of medical conditions. Drug delivery implants and pumps are generally utilized when a localized pharmaceutical impact is desired (i.e., the condition affects only a specific region) or when systemic delivery of the agent is inefficient or ineffective (i.e., leads to toxicity or severe side effects, results in inactivation of the drug prior to reaching the target tissue, produces poor symptom/disease control, and/or leads to addiction to the medication). Implantable pumps can also deliver systemic drug levels in a constant, regulated manner for extended periods and help patients avoid the “peaks and valleys” of blood-level drug concentrations associated with intermittent systemic dosing. Another advantage of implantable pumps is improved patient compliance. Many patients forget to take their medications regularly (particularly the young, elderly, chronically ill, mentally handicapped), but with an implantable pump, this problem is alleviated. For many patients this can lead to better symptom control (the dosage can often be titrated to the severity of the symptoms), superior disease management (particularly for insulin delivery in diabetics), and lower drug requirements (particularly for pain medications).

Innumerable drug delivery implants and pumps have been used in a variety of clinical applications, including programmable insulin pumps for the treatment of diabetes, intrathecal (in the spine) pumps to administer narcotics (e.g., morphine, fentanyl) for the relief of pain (e.g., cancer, back problems, HIV, post-surgery), local and systemic delivery of chemotherapy for the treatment of cancer (e.g., hepatic artery 5-FU infusion for liver tumors), medications for the treatment of cardiac conditions (e.g., anti-arrhythmic drugs for cardiac rhythm abnormalities), intrathecal delivery of anti-spasmotic drugs (e.g., baclofen) for spasticity in neurological disorders (e.g., Multiple Sclerosis, spinal cord injuries, brain injury, cerebral palsy), or local/regional antibiotics for infection management (e.g., osteomyelitis, septic arthritis). Typically, drug delivery pumps are implanted subcutaneously and consist of a pump unit with a drug reservoir and a flexible catheter through which the drug is delivered to the target tissue. The pump stores and releases prescribed amounts of medication via the catheter to achieve therapeutic drug levels either locally or systemically (depending upon the application). The center of the pump has a self-sealing access port covered by a septum such that a needle can be inserted percutaneously (through both the skin and the septum) to refill the pump with medication as required. There are generally two types of implantable drug delivery pumps. Constant-rate pumps are usually powered by gas and are designed to dispense drugs under pressure as a continual dosage at a preprogrammed, constant rate. The amount and rate of drug flow and regulated by the length of the catheter used, temperature, and altitude and they are best when unchanging, long-term drug delivery is required. Programmable-rate pumps utilize a battery-powered pump and a constant pressure reservoir to deliver drugs on a periodic basis in a manner that can be programmed by the physician or the patient. For the programmable infusion device, the drug may be delivered in small, discrete doses based on a programmed regimen which can be altered according to an individual's clinical response.

In general, drug delivery pumps are implanted to deliver drug at a regulated dose and may, in certain applications, be used in conjunction with implantable sensors that collect information which is used to regulate drug delivery (often called a “closed loop” system). Implantable drug delivery pumps may function and deliver drug in a variety of ways, which include, but are not limited to: (a) delivering drugs only when changes in the body are detected (e.g., sensor stimulated); (b) delivering drugs as a continuous slow release (e.g., constant flow);

(c) delivering drugs at prescribed dosages in a pulsatile manner (e.g., non-constant flow); (d) delivering drugs by programmable means; and (e) delivering drugs through a device that is designed for a specific anatomical site (e.g., 5 intraocular, intrathecal, intraperitoneal, intra-arterial or intracardiac). In addition to delivering drugs in a specific way or to a specific location, drug delivery pumps may also be categorized based on their mechanical delivery technology (e.g., the driving force by which drug delivery occurs). For example, the mechanics for delivering drugs may include, without limitation, osmotic pumps, metering systems, peristaltic (roller) pumps, electronically driven pumps, ocular drug delivery pumps and implants, elastomeric pumps, spring-contraction pumps, gas-driven pumps (e.g., induced by electrolytic cell or chemical reaction), hydraulic pumps, piston-dependent pumps and non-piston-dependent pumps, dispensing chambers, infusion pumps, passive pumps, infusate pumps and osmotically-driven fluid dispensers.

The clinical function of an implantable drug delivery device or pump depends upon the device, particularly the catheter or drug-dispensing component(s), being able to effectively maintain intimate anatomical contact with the target tissue (e.g., the sudural space in the spinal cord, the arterial lumen, the peritoneum, the interstitial fluid) and not becoming encapsulated or obstructed by scar tissue. Unfortunately, in many instances when these devices are implanted in the body, they are subject to a “foreign body” response from the surrounding host tissues as described previously. For implantable pumps, the drug-delivery catheter lumen, catheter tip, dispensing components, or delivery membrane may become obstructed by scar tissue which may cause the flow of drug to slowdown or cease completely. Alternatively, the entire pump, the catheter and/or the dispensing components can become encapsulated by scar (i.e., the body “walls off” the device with fibrous tissue) so that the drug is incompletely delivered to the target tissue (i.e., the scar prevents proper drug movement and distribution from the implantable pump to the tissues on the other side of the capsule). Either of these developments may lead to inefficient or incomplete drug flow to the desired target tissues or organs (and loss of clinical benefit), while encapsulation can also lead to local drug accumulation (in the capsule) and additional clinical complications (e.g., local drug toxicity; drug sequestration followed by sudden “dumping” of large amounts of drug into the surrounding tissues). Additionally, the tissue surrounding the implantable pump can be inadvertently damaged from the inflammatory foreign body response leading to loss of function and/or tissue damage (e.g., scar tissue in the spinal canal causing pain or obstructing the flow of cerebrospinal fluid). Implantation of an implantable drug delivery device or pump may also introduce or promote infection in the vicinity of the implant site.

Implantable drug delivery pumps that release one or more therapeutic agents for reducing scarring at the device-tissue interface (particularly in and around the drug delivery catheter or drug dispensing components) may help prolong the clinical performance of these devices. Inhibition of fibrosis can make sure that the correct amount of drug is dispensed from the device at the appropriate rate and that potentially toxic drugs do not become sequestered in a fibrous capsule. For devices that include electrical or battery components, not only can fibrosis cause the device to function suboptimally or not at all, it can cause excessive drain on battery life as increased energy is required to overcome the increased resistance imposed by the intervening scar tissue. Implantation of an implantable drug delivery device or pump may also introduce or promote infection in the vicinity of the implant site.

Virtually any implantable pump may benefit from the present invention. In one aspect, the drug delivery pump may deliver drugs in a continuous, constant-flow, slow release manner. For example, the drug delivery pump may be a passive pump adapted to provide a constant flow of medication which may be regulated by a pressure sensing chamber and a valve chamber in which the constant flow rate may be changed to a new constant flow rate. See, e.g., U.S. Pat. No. 6,589,205. In another aspect, the drug delivery pump may deliver drugs at prescribed dosages in a non-constant flow or pulsatile manner. For example, the drug delivery pump may adapt a regular pump to generate a pulsatile fluid drug flow by continuously filling a chamber and then releasing a valve to provide a bolus pulse of the drug. See, e.g., U.S. Pat. No. 6,312,409. In another aspect, the drug delivery pump may be programmed to dispense drug in a very specific manner. For example, the drug delivery pump may be a programmable infusate pump composed of a variable volume infusate chamber, and variable volume control fluid pressure and displacement reservoirs, whereby a fluid flow is sampled by a microprocessor based on the programmed value and adjustments are made accordingly to maintain the programmed fluid flow. See, e.g., U.S. Pat. No. 4,443,218.

In another aspect, the drug delivery pump suitable for use in the present invention may be manufactured based on different mechanical technologies (e.g., driving forces) of delivering drugs. For example, the drug delivery pump may be an implant composed of a piston that divides two chambers in which one chamber contains a water-swellable agent and the other chamber contains a leuprolide formulation for delivery. See, e.g., U.S. Pat. No. 5,728,396. The drug delivery pump may be a non-cylindrical osmotic pump system that may not rely upon a piston to infuse drug and conforms to the anatomical implant site. See, e.g., U.S. Pat. No. 6,464,688. The drug delivery pump may be an osmotically driven fluid dispenser composed of a flexible inner bag that contains the drug composition and a port in which the composition can be delivered. See, e.g., U.S. Pat. No. 3,987,790. The drug delivery pump may be a fluid-imbibing delivery implant composed of a compartment with a composition permeable to the passage of fluid and has an extended rigid sleeve to resist transient mechanical forces. See, e.g., U.S. Pat. Nos. 5,234,692 and 5,234,693. The drug delivery pump may be a pump with an isolated hydraulic reservoir, metering device, displacement reservoir, drug reservoir, and drug infusion port that is all contained in a housing apparatus. See, e.g., U.S. Pat. No. 6,629,954. The drug delivery pump may be composed of a dispensing chamber that has a dispensing passage and valves that are under compressive force to enable drug to flow in a one-way direction. See, e.g., U.S. Pat. No. 6,283,949. The drug delivery pump may be spring-driven based on a spring regulating pressure difference with a variable volume drug chamber. See, e.g., U.S. Pat. No. 4,772,263. Other examples of drug delivery pumps are described in, e.g., U.S. Pat. Nos. 6,645,176; 6,471,688; 6,283,949; 5,137,727 and 5,112,614.

Implantable drug delivery devices and pumps, which may benefit from having the subject polymer composition infiltrated into adjacent tissue according to the present invention, include commercially available products. For example, there are osmotically driven drug delivery pumps that are commercially available and suitable for the practice of the invention. These osmotic pumps include the DUROS Implant and ALZET Osmotic Pump from Alza Corporation (Mountain View, Calif.), which are used to delivery a wide variety of drugs and other therapeutics through the method of osmosis (see, e.g., U.S. Pat. Nos. 6,283,953; 6,270,787; 5,660,847; 5,112,614; 5,030,216 and 4,976,966).

As described above, infiltration of the subject polymer composition into tissue adjacent to the drug delivery pump can improve performance of the device and/or prevent or inhibit infection in the vicinity of the implant site. In one aspect, the present invention provides implantable drug delivery devices and pumps having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with implantable drug delivery devices and pumps have been described above.

Polymeric compositions may be infiltrated around implanted implantable drug delivery devices and pumps by applying the composition directly and/or indirectly into and/or onto (a) tissue adjacent to the implantable drug delivery device or pump; (b) the vicinity of the implantable drug delivery device or pump-tissue interface; (c) the region around the implantable drug delivery device or pump; and (d) tissue surrounding the implantable drug delivery device or pump. Methods for infiltrating the subject polymer compositions into tissue adjacent to an implantable drug delivery device or pump include delivering the polymer composition: (a) to the surface of the implantable drug delivery device or pump (e.g., as an injectable, paste, gel or mesh) during the implantation procedure; (b) to the surface of the tissue (e.g., as an injectable, paste, gel, in situ forming gel or mesh) immediately prior to, or during, implantation of the implantable drug delivery device or pump; (c) to the surface of the implantable drug delivery device or pump and/or the tissue surrounding the implanted implantable drug delivery device or pump (e.g., as an injectable, paste, gel, in situ forming gel or mesh) immediately after the implantation of the implantable drug delivery device or pump; (d) by topical application of the composition into the anatomical space where the implantable drug delivery device or pump may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the implantable drug delivery device or pump as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device, including the device only, pump only, catheter only, drug dispensing components only and/or a combination thereof.

According to one aspect, any fibrosis-inhibiting and/or anti-infective agent described above may be utilized in the practice of the present invention. In one aspect of the invention, the subject polymer compositions infiltrated into tissue adjacent to implantable drug delivery devices and pumps may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As implantable drug delivery devices and pumps are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1 000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm , or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

It should be obvious to one of skill in the art that commercial drug delivery pumps not specifically cited as well as next-generation and/or subsequently-developed commercial drug delivery products are to be anticipated and are suitable for use under the present invention.

Several specific drug delivery pumps and treatments will be described in greater detail below.

(1) Implantable Insulin Pumps for Diabetes

In one aspect, the subject polymer compositions may be infiltrated into tissue adjacent to an insulin pump. The subject polymer compositions may contain a therapeutic agent (e.g., an anti-scarring and/or anti-infective agent).

Insulin pumps are used for patients with diabetes to replace the need to control blood glucose levels by daily manual injections of insulin. Precise titration of the dosage and timing of insulin administration is a critical component in the effective management of diabetes. If the insulin dosage is too high, blood glucose levels drop precipitously, resulting in confusion and potentially even loss of consciousness. If insulin dosage is too low, blood glucose levels rise too high, leading to excessive thirst, urination, and changes in metabolism known as ketoacidosis. If the timing of insulin administration is incorrect, blood glucose levels can fluctuate wildly between the two extremes—a situation that is thought to contribute to some of the long-term complications of diabetes such as heart disease, kidney failure, nerve damage and blindness. Since in the extreme, all these conditions can be life threatening, the precise dosing and timing of insulin administration is essential to preventing the short and long-term complications of diabetes.

Implantable pumps automate the administration of insulin and eliminate human errors of dosage and timing that can have long-term health consequences. The pump has the capability to inject insulin regularly, multiple times a day and in small doses into the blood stream, peritoneal cavity or subcutaneous tissue. The pump is refilled with insulin once or twice a month by injection directly into the pump chamber. This reduces the number of externally administered injections the patient must undergo and also allows preprogrammed variable amounts of insulin to be released at different times into the blood stream; a situation which more closely resembles normal pancreas function and minimizes fluctuations in blood glucose levels. The insulin pump may be activated by an externally generated signal after the patient has withdrawn a drop of blood, subjected it to an analysis, and made a determination of the amount of insulin that needs to be delivered. However, the most widely pursued application of this technology is the production of a closed-loop “artificial pancreas” which can continuously detect blood glucose levels (through an implanted sensor) and provide feedback to an implantable pump to modulate the administration of insulin to a diabetic patient.

Numerous types of insulin pumps are suitable for use in the practice of the invention. For example, the drug delivery pump may include both an implantable sensor and a drug delivery pump by being composed of a mass of living cells and an electrical signal that regulates the delivery of glucose or glucagon or insulin. See, e.g., U.S. Pat. No. 5,474,552. The drug delivery pump may be composed of a single channel catheter with a sensor which is implanted in a vessel that transmits blood chemistry to a subcutaneously implanted infusion device which then dispenses medication through the catheter. See, e.g., U.S. Pat. No. 5,109,850.

Insulin pumps, which may benefit from having the subject polymer composition infiltrated into adjacent tissue according to the present invention, include commercially available products. Commercially available insulin pump devices suitable for the practice of the invention include the MINIMED 2007 Implantable Insulin Pump System from Medtronic MiniMed, Inc. (Northridge, Calif.). The MINIMED pump delivers insulin into the peritoneal cavity in short, frequent bursts to provide insulin to the body similar to that of the normal pancreas (see, e.g., U.S. Pat. Nos. 6,558,345 and 6,461,331). The MINIMED 2001 Implantable Insulin Pump System (Medtronic MiniMed Inc., Northridge, Calif.) delivers intraperitoneal insulin injections in a pulsatile manner from a negative pressure reservoir. Both these devices feature a long catheter that transports insulin from the subcutaneously implanted pump into the peritoneal cavity. As described above, the peritoneal drug-delivery catheter lumen or catheter tip may become partially or fully obstructed by scar tissue which may cause the flow of drug to slowdown or cease completely. Insulin pump devices such as these may also benefit from release of a therapeutic agent able to prevent or inhibit infection in the catheter and/or vicinity of the implant site. In one aspect of the present invention, the device includes delivery catheters having the subject polymer composition comprising an anti-scarring agent and/or anti-infective agent infiltrated into tissue adjacent to where the delivery catheter is or will be implanted to keep the delivery catheter lumen patent and/or prevent fibrosis in the surrounding tissue and/or inhibit or prevent infection in the catheter or vicinity of the implant site. In another aspect, the present invention provides insulin pumps having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with insulin pumps have been described above.

Polymeric compositions may be infiltrated around implanted insulin pumps by applying the composition directly and/or indirectly into and/or onto (a) tissue adjacent to the insulin pump; (b) the vicinity of the insulin pump-tissue interface; (c) the region around the insulin pump; and (d) tissue surrounding the insulin pump. Methods for infiltrating the subject polymer compositions into tissue adjacent to a insulin pump include delivering the polymer composition: (a) to the surface of the insulin pump (e.g., as an injectable, paste, gel or mesh) during the implantation procedure; (b) to the surface of the tissue (e.g., as an injectable, paste, gel, in situ forming gel or mesh) immediately prior to, or during, implantation of the insulin pump; (c) to the surface of the insulin pump and/or the tissue surrounding the implanted insulin pump (e.g., as an injectable, paste, gel, in situ forming gel or mesh) immediately after the implantation of the insulin pump; (d) by topical application of the composition into the anatomical space where the insulin pump may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the insulin pump as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device, including the device only, pump only, catheter only, drug dispensing components only and/or a combination thereof.

According to one aspect, any fibrosis-inhibiting and/or anti-infective agent described above may be utilized in the practice of the present invention. In one aspect of the invention, the subject polymer compositions infiltrated into tissue adjacent to insulin pumps may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As insulin pumps are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

It should be obvious to one of skill in the art that commercial drug delivery pumps not specifically cited as well as next-generation and/or subsequently-developed commercial drug delivery products are to be anticipated and are suitable for use under the present invention.

(2) Intrathecal Drug Delivery Pumps

In one aspect, the subject polymer compositions may be infiltrated into tissue adjacent to an intrathecal drug delivery pump. The subject polymer compositions may contain a therapeutic agent (e.g., an anti-scarring and/or anti-infective agent). Intrathecal drug delivery pumps having the subject polymer composition infiltrated into tissue adjacent to the pump may used to deliver drugs into the spinal cord for pain management and movement disorders.

Chronic pain is one of the most important clinical problems in all of medicine. For example, it is estimated that over 5 million people in the United States are disabled by back pain. The economic cost of chronic back pain is enormous, resulting in over 100 million lost work days annually at an estimated cost of $50-100 billion. The cost of managing pain for oncology patients is thought to approach $12 billion. Chronic pain disables more people than cancer or heart disease and costs the American public more than both cancer and heart disease combined. In addition to the physical consequences, chronic pain has numerous other costs including loss of employment, marital discord, depression and prescription drug addiction. It goes without saying, therefore, that reducing the morbidity and costs associated with persistent pain remains a significant challenge for the healthcare system.

Intractable severe pain resulting from injury, illness, scoliosis, spinal disc degeneration, spinal cord injury, malignancy, arachnoiditis, chronic disease, pain syndromes (e.g., failed back syndrome, complex regional pain syndrome) and other causes is a debilitating and common medical problem. In many patients, the continued use of analgesics, particularly drugs like narcotics, are not a viable solution due to tolerance, loss of effectiveness, and addiction potential. In an effort to combat this, intrathecal drug delivery devices have been developed to treat severe intractable back pain that is resistant to other traditional treatment modalities such as drug therapy, invasive therapy (surgery), or behavioral/lifestyle changes.

Intrathecal drug delivery pumps are designed and used to reduce pain by delivering pain medication directly into the cerebrospinal fluid of the intrathecal space surrounding the spinal cord. Typically, since this therapy delivers pain medication topically to pain receptors contained in the spinal cord that transmit pain sensation directly to the brain, smaller doses of medication are needed to gain relief. Morphine and other narcotics (usually fentanyl and sufentanil) are the most commonly delivered agents and many patients receive superior relief with lower doses than can be achieved with systemic delivery. Intrathecal drug delivery also allows the administration of pain medications (such as Ziconotide; an N-type calcium channel blocker made by Elan Pharmaceuticals) that cannot cross the blood-brain barrier and are thus only effective when administered by this route.

Intrathecal pumps are also used in the management of neurological and movement disorders. Baclofen (marketed as Lioresal by Novartis) is an antispasmotic/muscle relaxant used to treat spasticity and improve mobility in patients with Multiple Sclerosis, cystic fibrosis and spinal injuries. This drug has been proven to be more effective and cause fewer side effects when administered into the CSF by an intrathecal drug delivery pump. Efforts are also underway to treat epilepsy, brain tumors, Alzheimer's disease, Parkinson's disease and Amyetropic Lateral Sclerosis (ALS—Lou Gehrig's disease) via intrathecal administration of agents that may be too toxic to deliver systemically or do not cross the blood-brain barrier. For example, trials of intrathecally administered recombinant brain-derived neurotrophic factor (r-BDNF made by Amgen) have been undertaken in ALS patients.

An intrathecal drug delivery system consists of an intrathecal drug infusion pump and an intraspinal catheter, both of which are fully implanted. The pump device is implanted under the skin in the abdominal area, just above or below the beltline and can be refilled by percutaneous injection of the drug into the reservoir. The catheter is tunneled under the skin and runs from the pump to the intrathecal space of the spine. When operational, the pump administers prescribed amounts of medication to the cerebrospinal fluid in either a continuous fashion or in a manner than can be controlled by the physician or the patient in response to symptoms.

Numerous types of implantable intrathecal pumps are suitable for use in the practice of the invention. For example, the implantable pump used to deliver medication may be composed of two osmotic pumps with semipermeable membranes configured to deliver up to two drug delivery regimens at different rates, and having a built-in backup drug delivery system whereby the delivery of drug may continue when the primary delivery system reaches the end of its useful life or fails unexpectedly. See, e.g., U.S. Pat. No. 6,471,688. The implantable pump may be may be composed of a battery-operated pump unit with a drug reservoir, catheter, and electrodes that are implanted in the epidural space of a patient for relief of pain by delivering a liquid pain-relieving agent through the catheter to the desired location. See, e.g., U.S. Pat. No. 5,458,631.

Similar drug-delivery pumps have been described for the infusion of agents into regions of the brain to locally affect the excitability of the neurons in the treatment of a variety of chronic neurogenerative diseases (such as those described above for intrathecal delivery). Implantable pumps may be implanted abdominally which then dispenses drug through a catheter that is tunneled from the abdominal implant site, through the neck to an entry site in the head, and then to the localized treatment site within the brain. Pumps that deliver drug to the brain may discharge the drug at a variety of locations, including, but not limited to, anterior thalamus, ventrolateral thalamus, internal segment of the globus pallidus, substantia nigra pars reticulate, subthalamic nucleus, external segment of globus pallidus, and neostriatum. For example, the drug delivery pump may be composed of an implantable pump portion coupled to a catheter for infusing dosages of drug to a predetermined location of the brain when a sensor detects a symptom, such that a neurological disorder (e.g., seizure) may be treated. See, e.g., U.S. Pat. No. 5,978,702. The implantable pump may be implanted adjacent to a predetermined infusion site in a brain such that a predetermined dosage of at least one drug capable of altering the level of excitation of neurons of the brain may be infused such that neurodegeneration is prevented and/or treated. See, e.g., U.S. Pat. No. 5,735,814. The implantable pump may include a reservoir for the therapeutic agent which is stored between the galea aponeurotica and cranium of a subject whereby drug is then dispensed via pumping action to the desired location. See, e.g., U.S. Pat. No. 6,726,678.

Intrathecal drug delivery pumps, which may benefit from having the subject polymer composition infiltrated into adjacent tissue according to the present invention, include commercially available products. There are numerous commercially available implantable, intrathecal drug-delivery systems which are suitable for the practice of the invention. The SYNCHROMED EL Infusion System which is made by Medtronic, Inc. and is indicated for chronic Intrathecal Baclofen Therapy (ITB Therapy) (see, e.g., U.S. Pat. Nos. 6,743,204; 6,669,663; 6,635,048; 6,629,954; 6,626,867; 6,102,678; 5,978,702 and 5,820,589) The SYNCHROMED pump is a programmable, battery-operated device that stores and delivers medication based on the programmed dosing regimen. Medtronic, Inc. (Minneapolis, Minn.) also sells their ISOMED Constant-Flow Infusion System for use in delivering morphine sulfate directly into the intrathecal space as a treatment for chronic pain. Arrow International produces the Model 3000 infusion pump that provides constant-rate administration of agents such as morphine and baclofen into the intrathecal space. Tricumed Medizintechnik GmbH (Kiel, Germany) produces the Archimedes® constant flow implantable infusion pump for intrathecal administration of pain and antispasmotic drugs. Advanced Neuromodulation Systems (Plano, Tex.) produces the AccuRx® infusion pump for the treatment of pain and neuromuscular disorders. All these devices feature a long catheter that transports the active agent from a subcutaneously implanted pump into the intrathecal space in the spinal cord. As described above, the intrathecal drug-delivery catheter lumen or catheter tip may become partially or fully obstructed by scar tissue which may cause the flow of drug to slowdown or cease completely. Another potential complication with intrathecal drug delivery is the formation of fibrous tissue in the subdural space that can obstruct CSF flow and lead to serious complications (e.g., hydrocephalus, increased intracranial pressure). Intrathecal drug delivery devices such as these may also benefit from release of a therapeutic agent able to prevent or inhibit infection in the catheter and/or vicinity of the implant site. In one aspect of the present invention, the device includes delivery catheters having the subject polymer composition comprising an anti-scarring agent and/or anti-infective agent infiltrated into tissue adjacent to where the delivery catheter is or will be implanted to keep the delivery catheter lumen patent and/or prevent fibrosis in the surrounding tissue and/or inhibit or prevent infection in the catheter or vicinity of the implant site. In another aspect, the present invention provides intrathecal drug delivery devices having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with intrathecal drug delivery devices have been described above.

Polymeric compositions may be infiltrated around implanted intrathecal drug delivery devices by applying the composition directly and/or indirectly into and/or onto (a) tissue adjacent to the intrathecal drug delivery device; (b) the vicinity of the intrathecal drug delivery device-tissue interface; (c) the region around the intrathecal drug delivery device; and (d) tissue surrounding the intrathecal drug delivery device. Methods for infiltrating the subject polymer compositions into tissue adjacent to an intrathecal drug delivery device include delivering the polymer composition: (a) to the surface of the intrathecal drug delivery device (e.g., as an injectable, paste, gel or mesh) during the implantation procedure; (b) to the surface of the tissue (e.g., as an injectable, paste, gel, in situ forming gel or mesh) immediately prior to, or during, implantation of the intrathecal drug delivery device; (c) to the surface of the intrathecal drug delivery device and/or the tissue surrounding the implanted intrathecal drug delivery device (e.g., as an injectable, paste, gel, in situ forming gel or mesh) immediately after the implantation of the intrathecal drug delivery device; (d) by topical application of the composition into the anatomical space where the intrathecal drug delivery device may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the intrathecal drug delivery device as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device, including the device only, pump only, catheter only, drug dispensing components only and/or a combination thereof.

According to one aspect, any fibrosis-inhibiting and/or anti-infective agent described above may be utilized in the practice of the present invention. In one aspect of the invention, the subject polymer compositions infiltrated into tissue adjacent to intrathecal drug delivery devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCOA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As intrathecal drug delivery devices are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

It should be obvious to one of skill in the art that commercial intrathecal drug delivery pumps not specifically cited as well as next-generation and/or subsequently-developed commercial drug delivery products are to be anticipated and are suitable for use under the present invention.

(3) Implantable Drug Delivery Pumps for Chemotherapy

In one aspect, the subject polymer compositions may be infiltrated into tissue adjacent to a chemotherapeutic drug delivery pump. The subject polymer compositions may contain a therapeutic agent (e.g., an anti-scarring and/or anti-infective agent).

The drug delivery pump may be a pump that dispenses a chemotherapeutic drug for the treatment of cancer. Pumps for dispensing a drug for the treatment of cancer are used to deliver chemotherapeutic agents to a local area of the body. Although virtually any malignancy may potentially be treated in this manner (i.e., by infusing drug directly into a solid tumor or into the blood vessels that supply the tumor), current treatments revolve around the management of hepatic (liver) tumors. For example, FUDR (2′-deoxy 5-fluorouridine) is used in the palliative management of adenocarcinoma (colon, breast, stomach) that has metastasized to the liver. In hepatic artery infusion therapy the drug is delivered via an implantable pump into the artery which provides blood supply to the liver. This allows for higher drug concentrations to reach the liver (the drug is not diluted in the blood as may occur in intravenous administration) and prevents clearance by the liver (the drug is metabolized by the liver and may be rapidly cleared from the bloodstream if administered i.v.); both of which allow higher concentrations of the drug to reach the tumor.

Numerous types of implantable pumps are suitable for delivering chemotherapeutic agents in the practice of the invention. For example, the implantable pump may have a dispensing chamber with a dispensing passage and actuator, reservoir housing with reservoir, and septum for refilling the reservoir. See, e.g., U.S. Pat. No. 6,283,949. Medtronic, Inc. sells their ISOMED Constant-Flow Infusion System which may be used to deliver chronic intravascular infusion of floxuridine in a fixed flow rate for the treatment of primary or metastatic cancer. Tricumed Medizintechnik GmbH (Kiel, Germany) sells their ARCHIMEDES DC implantable infusion pump specially adapted to deliver chemotherapy in a constant flow rate within the vicinity of a tumor (see, e.g., U.S. Pat. Nos. 5,908,414 and 5,769,823). Arrow International produces the Model 3000 infusion pump that provides constant-rate administration of chemotherapeutic agents into a tumor. All these devices feature a catheter that transports the chemotherapeutic agent from a subcutaneously implanted pump directly into the tumor or the artery that supplies a tumor. As described above, the drug-delivery catheter lumen or catheter tip may become partially or fully obstructed by scar tissue which may cause the flow of drug to slowdown or cease completely. If placed intravascularly, the drug-delivery catheter lumen or catheter tip may become partially or fully obstructed by neointimal tissue which may impair the flow of drug into the blood vessel. Chemotherapeutic drug delivery pumps such as these may also benefit from release of a therapeutic agent able to prevent or inhibit infection in the catheter and/or vicinity of the implant site. In one aspect of the present invention, the device includes delivery catheters having the subject polymer composition comprising an anti-scarring agent and/or anti-infective agent infiltrated into tissue adjacent to where the delivery catheter is or will be implanted to keep the delivery catheter lumen patent and/or prevent fibrosis in the surrounding tissue and/or inhibit or prevent infection in the catheter or vicinity of the implant site. In another aspect, the present invention provides chemotherapeutic drug delivery pumps having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with chemotherapeutic drug delivery pumps have been described above.

Polymeric compositions may be infiltrated around implanted chemotherapeutic drug delivery pumps by applying the composition directly and/or indirectly into and/or onto (a) tissue adjacent to the chemotherapeutic drug delivery pump; (b) the vicinity of the chemotherapeutic drug delivery pump-tissue interface; (c) the region around the chemotherapeutic drug delivery pump; and (d) tissue surrounding the chemotherapeutic drug delivery pump. Methods for infiltrating the subject polymer compositions into tissue adjacent to a chemotherapeutic drug delivery pump include delivering the polymer composition: (a) to the surface of the chemotherapeutic drug delivery pump (e.g., as an injectable, paste, gel or mesh) during the implantation procedure; (b) to the surface of the tissue (e.g., as an injectable, paste, gel, in situ forming gel or mesh) immediately prior to, or during, implantation of the chemotherapeutic drug delivery pump; (c) to the surface of the chemotherapeutic drug delivery pump and/or the tissue surrounding the implanted chemotherapeutic drug delivery pump (e.g., as an injectable, paste, gel, in situ forming gel or mesh) immediately after the implantation of the chemotherapeutic drug delivery pump; (d) by topical application of the composition into the anatomical space where the chemotherapeutic drug delivery pump may be placed (particularly useful for this embodiment is the use of polymeric carriers which release the therapeutic agent over a period ranging from several hours to several weeks - fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the chemotherapeutic drug delivery pump as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device, including the device only, pump only, catheter only, drug dispensing components only and/or a combination thereof.

According to one aspect, any fibrosis-inhibiting and/or anti-infective agent described above may be utilized in the practice of the present invention. In one aspect of the invention, the subject polymer compositions infiltrated into tissue adjacent to chemotherapeutic drug delivery pumps may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCOA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As chemotherapeutic drug delivery pumps are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

It should be obvious to one of skill in the art that commercial chemotherapy delivery pumps and implants not specifically cited as well as next-generation and/or subsequently-developed commercial chemotherapy delivery products are to be anticipated and are suitable for use in the present invention.

(4) Drug Delivery Pumps for the Treatment of Heart Disease

In one aspect, the subject polymer compositions may be infiltrated into tissue adjacent to a drug delivery pump for the treatment of heart disease. The subject polymer compositions may contain a therapeutic agent (e.g., an anti-scarring and/or anti-infective agent).

The drug delivery pump may be a pump that dispenses a drug for the treatment of heart disease. Pumps for dispensing a drug for the treatment of heart disease may be used to treat conditions including, but not limited to atrial fibrillation and other cardiac rhythm disorders. Atrial fibrillation is a form of heart disease that afflicts millions of people. It is a condition in which the normal coordinated contraction of the heart is disrupted, primarily by abnormal and uncontrolled action of the atria of the heart. Normally, contractions occur in a controlled sequence with the contractions of the other chambers of the heart. When the right atrium fails to contract, contracts out of sequence, or contracts ineffectively, blood flow from the atria to the ventricles is disrupted. Atrial fibrillation can cause weakness, shortness of breath, angina, lightheadedness and other symptoms due to reduced ventricular filling and reduced cardiac output. Stroke can occur as a result of clot forming in a poorly contracting atria, breaking loose, and traveling via the bloodstream to the arteries of the brain where they become wedged and obstruct blood flow (which may lead to brain damage and death). Typically, atrial fibrillation is treated by medical or electrical conversion (defibrillation), however, complications may exist whereby the therapy causes substantial pain or has the potential to initiate a life threatening ventricular arrhythmia. The pain associated with the electrical shock is severe and unacceptable for many patients, since they are conscious and alert when the device delivers electrical therapy. Medical therapy involves the delivery of anti-arrhythmic drugs by injecting them intravenously, administering them orally or delivering them locally via a drug delivery pump.

Numerous types of implantable pumps are described for dispensing a drug for the treatment of heart disease and are suitable for use in the practice of the invention. For example, the drug delivery pump may be an implantable cardiac electrode which delivers stimulation energy and dispenses drug adjacent to the stimulation site. See, e.g., U.S. Pat. No. 5,496,360. The drug delivery pump may have a plurality of silicone septii to facilitate the filling of drug reservoirs within the pump which is subcutaneously implanted with a catheter which travels transvenously by way of the subclavian vein through the superior vena cava and into the right atrium for drug delivery. See, e.g., U.S. Pat. No. 6,296,630. As described above, the drug-delivery catheter lumen or catheter tip may become partially or fully obstructed by scar tissue which may cause the flow of drug to slowdown or cease completely. If placed intravascularly, the drug-delivery catheter lumen or catheter tip may become partially or fully obstructed by neointimal tissue which may impair the flow of drug into the blood vessel or the right atrium. Drug delivery pumps such as these may also benefit from release of a therapeutic agent able to prevent or inhibit infection in the catheter and/or vicinity of the implant site. In one aspect of the present invention, the device includes delivery catheters having the subject polymer composition comprising an anti-scarring agent and/or anti-infective agent infiltrated into tissue adjacent to where the delivery catheter is or will be implanted to keep the delivery catheter lumen patent and/or prevents fibrosis in the surrounding tissue and/or inhibit or prevent infection in the catheter or vicinity of the implant site. In another aspect, the present invention provides drug delivery pumps for the treatment of heart disease having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with drug delivery pumps for the treatment of heart disease have been described above.

Polymeric compositions may be infiltrated around implanted drug delivery pumps for the treatment of heart disease by applying the composition directly and/or indirectly into and/or onto (a) tissue adjacent to the drug delivery pump for the treatment of heart disease; (b) the vicinity of the drug delivery pump for the treatment of heart disease-tissue interface; (c) the region around the drug delivery pump for the treatment of heart disease; and (d) tissue surrounding the drug delivery pump for the treatment of heart disease. Methods for infiltrating the subject polymer compositions into tissue adjacent to a drug delivery pump for the treatment of heart disease include delivering the polymer composition: (a) to the surface of the drug delivery pump for the treatment of heart disease (e.g., as an injectable, paste, gel or mesh) during the implantation procedure; (b) to the surface of the tissue (e.g., as an injectable, paste, gel, in situ forming gel or mesh) immediately prior to, or during, implantation of the drug delivery pump for the treatment of heart disease; (c) to the surface of the drug delivery pump for the treatment of heart disease and/or the tissue surrounding the implanted drug delivery pump for the treatment of heart disease (e.g., as an injectable, paste, gel, in situ forming gel or mesh) immediately after the implantation of the drug delivery pump for the treatment of heart disease; (d) by topical application of the composition into the anatomical space where the drug delivery pump for the treatment of heart disease may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the drug delivery pump for the treatment of heart disease as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device, including the device only, pump only, catheter only, drug dispensing components only and/or a combination thereof.

According to one aspect, any fibrosis-inhibiting and/or anti-infective agent described above may be utilized in the practice of the present invention. In one aspect of the invention, the subject polymer compositions infiltrated into tissue adjacent to drug delivery pumps for the treatment of heart disease may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As drug delivery pumps for the treatment of heart disease are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm² _ 1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

It should be obvious to one of skill in the art that commercial cardiac drug delivery pumps not specifically cited as well as next-generation and/or subsequently-developed commercial cardiac drug delivery products are to be anticipated and are suitable for use under the present invention.

(5) Other Drug Delivery Implants

In one aspect, the subject polymer compositions may be infiltrated into tissue adjacent to an implantable pump for continuous delivery of pharmaceutical agents. The subject polymer compositions may contain a therapeutic agent (e.g., an anti-scarring and/or anti-infective agent).

Several other implantable pumps useful in the present invention have been developed for continuous delivery of pharmaceutical agents. For example, Debiotech S.A. (Switzerland) has developed the MIP device which is an implantable piezo-actuated silicon micropump for programmable drug delivery applications. This high-performance micropump is based on a MEMS (Micro-Electro-Mechanical) system which allows it to maintain a low flow rate. The DUROS sufentanil implant from Durect Corporation (Cupertino, Calif.) is a titanium cylinder that contains a drug reservoir, and a piston driven by an osmotic engine. The VIADUR (leuprolide acetate) implant available from Alza Corporation (Mountain View, Calif.) uses the same DUROS implant technology to deliver leuprolide over a 12 month period to reduces testosterone levels for the treatment prostate cancer (see, e.g., U.S. Pat. Nos. 6,283,953; 6,270,787; 5,660,847; 5,112,614; 5,030,216 and 4,976,966). Fibrous encapsulation of the device can cause failure in a number of ways including: obstructing the semipermeable membrane (which will impair functioning of the osmotic engine by preventing the flow of fluids into the engine), obstructing the exit port (which will impair drug flow out of the device) and/or complete encapsulation (which will create a microenvironment that prevents drug distribution). Many other drug delivery implants, osmotic pumps and the like suffer from similar problems—fibrous encapsulation prevents the appropriate release of drugs into the surrounding tissues. Drug delivery devices such as these may also benefit from release of a therapeutic agent able to prevent or inhibit infection in the catheter and/or vicinity of the implant site. In one aspect of the present invention, drug delivery devices having the subject polymer composition comprising an anti-scarring agent and/or anti-infective agent infiltrated into tissue adjacent to where the device is or will be implanted to prevent or inhibit encapsulation, prevent obstruction of the semipermeable membrane, keep the delivery catheter lumen patent, prevent fibrosis in the surrounding tissue and/or inhibit or prevent infection in the catheter or vicinity of the implant site. In one aspect, the present invention provides implantable pumps for continuous delivery of pharmaceutical agents having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with implantable pumps for continuous delivery of pharmaceutical agents have been described above.

Polymeric compositions may be infiltrated around implanted implantable pumps for continuous delivery of pharmaceutical agents by applying the composition directly and/or indirectly into and/or onto (a) tissue adjacent to the implantable pump for continuous delivery of pharmaceutical agents; (b) the vicinity of the implantable pump for continuous delivery of pharmaceutical agents-tissue interface; (c) the region around the implantable pump for continuous delivery of pharmaceutical agents; and (d) tissue surrounding the implantable pump for continuous delivery of pharmaceutical agents. Methods for infiltrating the subject polymer compositions into tissue adjacent to an implantable pump for continuous delivery of pharmaceutical agents include delivering the polymer composition: (a) to the surface of the implantable pump for continuous delivery of pharmaceutical agents (e.g., as an injectable, paste, gel or mesh) during the implantation procedure; (b) to the surface of the tissue (e.g., as an injectable, paste, gel, in situ forming gel or mesh) immediately prior to, or during, implantation of the implantable pump for continuous delivery of pharmaceutical agents; (c) to the surface of the implantable pump for continuous delivery of pharmaceutical agents and/or the tissue surrounding the implanted implantable pump for continuous delivery of pharmaceutical agents (e.g., as an injectable, paste, gel, in situ forming gel or mesh) immediately after the implantation of the implantable pump for continuous delivery of pharmaceutical agents; (d) by topical application of the composition into the anatomical space where the implantable pump for continuous delivery of pharmaceutical agents may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the implantable pump for continuous delivery of pharmaceutical agents as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the device, including the device only, pump only, catheter only, drug dispensing components only and/or a combination thereof.

According to one aspect, any fibrosis-inhibiting and/or anti-infective agent described above may be utilized in the practice of the present invention. In one aspect of the invention, the subject polymer compositions infiltrated into tissue adjacent to implantable pumps for continuous delivery of pharmaceutical agents may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As implantable pumps for continuous delivery of pharmaceutical agents are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

Although numerous implantable pumps have been described above, all possess similar design features and cause similar unwanted fibrous tissue reactions following implantation and may introduce or promote infection in the area of the implant site. It should be obvious to one of skill in the art that commercial sensor devices not specifically cited above as well as next-generation and/or subsequently-developed commercial implantable pump products are to be anticipated and are suitable for use under the present invention. The clinical function of an implantable drug delivery device or pump depends upon the device, particularly the catheter or drug-dispensing component(s), being able to effectively maintain intimate anatomical contact with the target tissue (e.g., the sudural space in the spinal cord, the arterial lumen, the peritoneum, the interstitial fluid) and not becoming encapsulated or obstructed by scar tissue. For implantable pumps, the drug-delivery catheter lumen, catheter tip, dispensing components, or delivery membrane may become obstructed by scar tissue which may cause the flow of drug to slowdown or cease completely. Alternatively, the entire pump, the catheter and/or the dispensing components can become encapsulated by scar (i.e., the body “walls off” the device with fibrous tissue) so that the drug is incompletely delivered to the target tissue (i.e., the scar prevents proper drug movement and distribution from the implantable pump to the tissues on the other side of the capsule). Either of these developments may lead to inefficient or incomplete drug flow to the desired target tissues or organs (and loss of clinical benefit), while encapsulation can also lead to local drug accumulation (in the capsule) and additional clinical complications (e.g., local drug toxicity; drug sequestration followed by sudden “dumping” of large amounts of drug into the surrounding tissues). For implantable pumps that include electrical or battery components, not only can fibrosis cause the device to function suboptimally or not at all, it can cause excessive drain on battery life as increased energy is required to overcome the increased resistance imposed by the intervening scar tissue. Implantable pumps that release a therapeutic agent for reducing scarring at the device-tissue interface can be used to increase efficacy, prolong clinical performance, ensure that the correct amount of drug is dispensed from the device at the appropriate rate, and reduce the risk that potentially toxic drugs become sequestered in a fibrous capsule. Implantable sensor devices may also benefit from release of a therapeutic agent able to prevent or inhibit infection in the vicinity of the implant site. In one aspect, the present invention provides implantable pumps having the subject polymer compositions infiltrated into adjacent tissue, where the subject polymer compositions may include a therapeutic agent (e.g., an anti-scarring and/or anti-infective agent). These compositions may further include one or more fibrosis-inhibiting agents such that the overgrowth of granulation or fibrous tissue is inhibited or reduced and/or one or more anti-infective agents such that infection in the vicinity of the implant site is inhibited or prevented.

Soft Tissue Implants

In one aspect, the subject polymer compositions may be infiltrated into tissue adjacent to a soft tissue implant. The subject polymer compositions may contain a therapeutic agent (e.g., an anti-scarring and/or anti-infective agent).

There are numerous types of soft tissue implants where the occurrence of a fibrotic reaction will adversely affect the functioning or appearance of the implant or the tissue surrounding the implant. Typically, fibrotic encapsulation of the soft tissue implant (or the growth of fibrous tissue between the implant and the surrounding tissue) can result in fibrous contracture and other problems that can lead to suboptimal appearance and patient comfort. Accordingly, the present invention provides for soft tissue implants having the subject polymer composition comprising an anti-scarring agent and/or anti-infective agent infiltrated into adjacent tissue to inhibit the formation of scar tissue to minimize or prevent encapsulation (and associated fibrous contracture) of the soft tissue implant and/or to inhibit or prevent infection in the vicinity of the implant site.

Soft tissue implants are used in a variety of cosmetic, plastic, and reconstructive surgical procedures and may be delivered to many different parts of the body, including, without limitation, the face, nose, breast, chin, buttocks, chest, lip and cheek. Soft tissue implants are used for the reconstruction of surgically or traumatically created tissue voids, augmentation of tissues or organs, contouring of tissues, the restoration of bulk to aging tissues, and to correct soft tissue folds or wrinkles (rhytides). Soft tissue implants may be used for the augmentation of tissue for cosmetic (aesthetic) enhancement or in association with reconstructive surgery following disease or surgical resection. Representative examples of soft tissue implants, which may benefit from having the subject polymer composition infiltrated into adjacent tissue according to the present invention, include, e.g., saline breast implants, silicone breast implants, triglyceride-filled breast implants, chin and mandibular implants, nasal implants, cheek implants, lip implants, and other facial implants, pectoral and chest implants, malar and submalar implants, and buttocks implants.

Soft tissue implants have numerous constructions and may be formed of a variety of materials, such as to conform to the surrounding anatomical structures and characteristics. In one aspect, soft tissue implants suitable for use in the present invention are formed from a polymer such as silicone, poly(tetrafluoroethylene), polyethylene, polyurethane, polymethylmethacrylate, polyester, polyamide and polypropylene. Soft tissue implants may be in the form shell (or envelope) that is filled with a fluid material such as saline.

In one aspect, soft tissue implants include or are formed from silicone or dimethylsiloxane. Silicone implants can be solid, yet flexible and very durable and stable. They are manufactured in different durometers (degrees of hardness) to be soft or quite hard, which is determined by the extent of polymerization. Short polymer chains result in liquid silicone with less viscosity, while lengthening the chains produces gel-type substances, and cross-linking of the polymer chains results in high-viscosity silicone rubber. Silicone may also be mixed as a particulate with water and a hydrogel carrier to allow for fibrous tissue ingrowth. These implants are designed to enhance soft tissue areas rather than the underlying bone structure. In certain aspects, silicone-based implants (e.g., chin implants) may be affixed to the underlying bone by way of one or several titanium screws. Silicone implants can be used to augment tissue in a variety of locations in the body, including, for example, breast, nasal, chin, malar (e.g., cheek), and chest/pectoral area. Silicone gel with low viscosity has been primarily used for filling breast implants, while high viscosity silicone is used for tissue expanders and outer shells of both saline-filled and silicone-filled breast implants. For example, breast implants are manufactured by both Inamed Corporation (Santa Barbara, Calif.) and Mentor Corporation (Santa Barbara, Calif.).

In another aspect, soft tissue implants include or are formed from poly(tetrafluoroethylene) (PTFE). In certain aspects, the poly(tetrafluoroethylene) is expanded polytetrafluoroethylene (ePTFE). PTFE used for soft tissue implants may be formed of an expanded polymer of solid PTFE nodes with interconnecting, thin PTFE fibrils that form a grid pattern, resulting in a pliable, durable, biocompatible material. Soft tissue implants made of PTFE are often available in sheets that may be easily contoured and stacked to a desired thickness, as well as solid blocks. These implants are porous and can become integrated into the surrounding tissue which aids in maintaining the implant in its appropriate anatomical location. PTFE implants generally are not as firm as silicone implants. Further, there is less bone resorption underneath ePTFE implants as opposed to silicone implants. Soft tissue implants composed of PTFE may be used to augment tissue in a variety of locations in the body, including, for example, facial, chest, lip, nasal, and chin, as well as the mandibular and malar region and for the treatment of nasolabial and glabellar creases. For example, GORE-TEX (W.L. Gore & Associates, Inc., Newark, Del.) is an expanded synthetic PTFE that may be used to form facial implants for augmentation purposes.

In yet another aspect, soft tissue implants include or are formed from polyethylene. Polyethylene implants are frequently used, for example in chin augmentation. Polyethylene implants can be porous, such that they may become integrated into the surrounding tissue, which provides an alternative to using titanium screws for stability. Polyethylene implants may be available with varying biochemical properties, including chemical resistance, tensile strength, and hardness. Polyethylene implants may be used for facial reconstruction, including malar, chin, nasal, and cranial implants. For example, Porex Surgical Products Group (Newnan, Ga.) makes MEDPOR which is a high-density, porous polyethylene implant that is used in facial reconstruction. The porosity allows for vascular and soft tissue ingrowth for incorporation of the implant.

In yet another aspect, soft tissue implants include or are formed from polypropylene. Polypropylene implants are a loosely woven, high density polymer having similar properties to polyethylene. These implants have good tensile strength and are available as a woven mesh, such as PROLENE (Ethicon, Inc., Sommerville, N.J.) or MARLEX (C.R. Bard, Inc., Billerica, Mass.). Polypropylene implants may be used, for example, as chest implants.

In yet another aspect, soft tissue implants include or are formed from polyamide. Polyamide is a nylon compound that is woven into a mesh that may be implanted for use in facial reconstruction and augmentation. These implants are easily shaped and sutured and undergo resorption over time. SUPRAMID and SUPRAMESH (S. Jackson, Inc., Minneapolis, Minn.) are nylon-based products that may be used for augmentation, however, because of their resorptive properties, their application is limited.

In yet another aspect, soft tissue implants include or are formed from polyester. Nonbiodegradable polyesters, such as MERSILENE Mesh (Ethicon, Inc.) and DACRON (available from Invista, Wichita, Kans.), may be suitable as implants for applications that require both tensile strength and stability, such as chest, chin and nasal augmentation.

In yet another aspect, soft tissue implants include or are formed from polymethylmethacrylate. These implants have a high molecular weight and have compressive strength and rigidity even though they have extensive porosity. Polymethylmethacrylate, such as Hard Tissue Replacement (HTR) polymer made by U.S. Surgical Corporation (Norwalk, Conn.), may be used for chin and malar augmentation as well as craniomaxillofacial reconstruction.

In yet another aspect, soft tissue implants include or are formed from polyurethane. Polyurethane may be used as a foam to cover breast implants. This polymer promotes tissue ingrowth resulting in low capsular contracture rate in breast implants.

Examples of commercially available polymeric soft tissue implants, which may benefit from having the subject polymer composition infiltrated into adjacent tissue according to the present invention, include silicone implants from Surgiform Technology, Ltd. (Columbia Station, Ohio); ImplantTech Associates (Ventura, Calif.); Inamed Corporation (Santa Barbara, Calif.; see M766A Spectrum Catalog); Mentor Corporation (Santa Barbara, Calif.); and Allied Biomedical (Ventura, Calif.). Saline filled breast implants are made by both Inamed and Mentor and may also benefit from implantation in combination with a fibrosis inhibitor. Commercially available poly(tetrafluoroethylene) soft tissue implants suitable for use in combination with a fibrosis-inhibitor include poly(tetrafluoroethylene) cheek, chin, and nasal implants from W. L. Gore & Associates, Inc. (Newark, Del.). Commercially available polyethylene soft tissue implants, which may benefit from having the subject polymer composition infiltrated into adjacent tissue according to the present invention, include polyethylene implants from Porex Surgical Inc. (Fairburn, Ga.) sold under the trade name MEDPOR Biomaterial. MEDPOR Biomaterial is composed of porous, high-density polyethylene material with an omni-directional latticework of interconnecting pores, which allows for integration into host tissues.

Upon implantation, excessive scar tissue growth can occur around the all or parts of the implant, which can lead to a reduction in the performance of these devices (as described previously). Soft tissue implants having the subject polymer compositions infiltrated into tissue adjacent to the implant site can be used to enhance the appearance, increase the longevity, reduce the need for corrective surgery or repeat procedures, decrease the incidence of pain and other symptoms, and improve the clinical function of implant. Soft tissue implants may also benefit from release of a therapeutic agent to prevent or inhibit infection in the vicinity of the implant site. Accordingly, in one aspect, the present invention provides soft tissue implants having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with soft tissue implants have been described above.

Polymeric compositions may be infiltrated around implanted soft tissue implants by applying the composition directly and/or indirectly into and/or onto (a) tissue adjacent to the soft tissue implant; (b) the vicinity of the soft tissue implant-tissue interface; (c) the region around the soft tissue implant; and (d) tissue surrounding the soft tissue implant. Methods for infiltrating the subject polymer compositions into tissue adjacent to a soft tissue implant include delivering the polymer composition: (a) to the surface of the soft tissue implant (e.g., as an injectable, paste, gel or mesh) during the implantation procedure; (b) to the surface of the tissue (e.g., as an injectable, paste, gel, in situ forming gel or mesh) immediately prior to, or during, implantation of the soft tissue implant; (c) to the surface of the soft tissue implant and/or the tissue surrounding the implanted soft tissue implant (e.g., as an injectable, paste, gel, in situ forming gel or mesh) immediately after the implantation of the soft tissue implant; (d) by topical application of the composition into the anatomical space where the soft tissue implant may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the implant may be inserted); (e) via percutaneous injection into the tissue surrounding the soft tissue implant as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the implant.

According to one aspect, any fibrosis-inhibiting and/or anti-infective agent described above may be utilized in the practice of the present invention. In one aspect of the invention, the subject polymer compositions infiltrated into tissue adjacent to soft tissue implants may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As soft tissue implants are made in a variety of configurations and sizes, the exact dose administered will also vary with implant size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the implant, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of implant or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the implant, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of implant or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

For greater clarity, several specific soft tissue implants and treatments will be described in greater detail below, including breast implants and other cosmetic implants.

(1) Breast Implants

In one aspect, the subject polymer compositions may be infiltrated into tissue adjacent to a breast implant. The subject polymer compositions may contain a therapeutic agent (e.g., an anti-scarring and/or anti-infective agent).

Breast implant placement for augmentation or breast reconstruction after mastectomy is one of the most frequently performed cosmetic surgery procedures. For example, in 2002 alone, over 300,000 women had breast implant surgery. Of these women, approximately 80,000 had breast reconstructions following a mastectomy due to cancer. An increased number of breast implant surgeries is highly likely given the incidence of breast cancer and current trends in cosmetic surgery.

In general, breast augmentation or reconstructive surgery involves the placement of a commercially available breast implant, which consists of a capsule filled with either saline or silicone, into the tissues underneath the mammary gland. Four different incision sites have historically been used for breast implantation: axillary (armpit), periareolar (around the underside of the nipple), inframamary (at the base of the breast where it meets the chest wall) and transumbilical (around the belly button). The tissue is dissected away through the small incision, often with the aid of an endoscope (particularly for axillary and transumbilical procedures where tunneling from the incision site to the breast is required). A pocket for placement of the breast implant is created in either the subglandular or the subpectorial region. For subglandular implants, the tissue is dissected to create a space between the glandular tissue and the pectoralis major muscle that extends down to the inframammary crease. For subpectoral implants, the fibres of the pectoralis major muscle are carefully dissected to create a space beneath the pectoralis major muscle and superficial to the rib cage. Careful hemostasis is essential (since it can contribute to complications such as capsular contractures), so much so that minimally invasive procedures (axillary, transumbilical approaches) must be converted to more open procedures (such as periareolar) if bleeding control is inadequate. Depending upon the type of surgical approach selected, the breast implant is often deflated and rolled up for placement in the patient. After accurate positioning is achieved, the implant can then be filled or expanded to the desired size.

Although many patients are satisfied with the initial procedure, significant percentages suffer from complications that frequently require a repeat intervention to correct. Encapsulation of a breast prosthesis that creates a periprosthetic shell (called capsular contracture) is the most common complication reported after breast enlargement, with up to 50% of patients reporting some dissatisfaction. Calcification can occur within the fibrous capsule adding to its firmness and complicating the interpretation of mammograms. Multiple causes of capsular contracture have identified including: foreign body reaction, migration of silicone gel molecules across the capsule and into the tissue, autoimmune disorders, genetic predisposition, infection, hematoma, and the surface characteristics of the prosthesis. Although no specific etiology has been repeatedly identified, at the cellular level, abnormal fibroblast activity stimulated by a foreign body is a consistent finding. Periprosthetic capsular tissues contain macrophages and occasional T- and B-lymphocytes, suggesting an inflammatory component to the process. Implant surfaces have been made both smooth and textured in an attempt to reduce encapsulation, however, neither has been proven to produce consistently superior results. Animal models suggest that there is an increased tendency for increased capsular thickness and contracture with textured surfaces that encourage fibrous tissue ingrowth on the surface. Placement of the implant in the subpectoral location appears to decrease the rate of encapsulation in both smooth and textured implants.

From a patient's perspective, the biological processes described above lead to a series of commonly described complaints. Implant malposition, hardness and unfavorable shape are the most frequently sited complications and are most often attributed to capsular contracture. When the surrounding scar capsule begins to harden and contract, it results in discomfort, weakening of the shell, asymmetry, skin dimpling and malpositioning. True capsular contractures will occur in approximately 10% of patients after augmentation, and in 25% to 30% of reconstruction cases, with most patients reporting dissatisfaction with the aesthetic outcome. Scarring leading to asymmetries occurs in 10% of augmentations and 30% of reconstructions and is the leading cause of revision surgery. Skin wrinkling (due to the contracture pulling the skin in towards the implant) is a complication reported by 10% to 20% of patients. Scarring has even been implicated in implant deflation (1-6% of patients; saline leaking out of the implant and “deflating” it), when fibrous tissue ingrowth into the diaphragmatic valve (the access site used to inflate the implant) causes it to become incontinent and leak. In addition, over 15% of patients undergoing augmentation will suffer from chronic pain and many of these cases are ultimately attributable to scar tissue formation. Other complications of breast augmentation surgery include late leaks, hematoma (approximately 1-6% of patients), seroma (2.5%), hypertrophic scarring (2-5%) and infections (about 1-4% of cases).

Overt implant infection (occurs in about 1-4% of cases) resulting from wound infections, contaminated saline in the implant, contamination of the breast implant at the time of surgical implantation and other causes necessitates the removal of the implant. Release of an anti-infective agent into the tissue surrounding an implant may reduce the incidence of breast implant infections and help prevent the formation of infection-induced capsular contracture.

Correction can involve several options including removal of the implant, capsulotomy (cutting or surgically releasing the capsule), capsulectomy (surgical removal of the fibrous capsule), or placing the implant in a different location (i.e., from subglandular to subpectoral). Ultimately, additional surgery (revisions, capsulotomy, removal, re-implantation) is required in over 20% of augmentation patients and in over 40% of reconstruction patients, with scar formation and capsular contracture being far and away the most common cause. Procedures to break down the scar may not be sufficient, and approximately 8% of augmentations and 25% of reconstructions ultimately have the implant surgically removed. Infiltration of the subject polymer composition comprising an anti-scarring agent and/or anti-infective agent into tissue adjacent to where the breast implant is or will be implanted can minimize fibrous tissue formation, encapsulation, capsular contracture and/or inhibit or prevent infection in the vicinity of the implant site. For example, attempts have been made to administer steroids either from the breast implant, or infiltrated into the intended mammary pocket, but this resulted in soft tissue atrophy and deformity. An ideal fibrosis-inhibiting agent will target only the components of the fibrous capsule and not harm the surrounding soft tissues. Infiltration of the subject polymer composition into tissue adjacent to the breast implant site may minimize or prevent fibrous contracture in response to gel or saline-containing breast implants that are placed subpectorally or subglandularly. Infiltration of the subject polymer composition into tissue adjacent to the breast implant site, including the tissue surrounding the breast implant or the surgical pocket where the implant will be placed, may prevent the formation of scar and capsular contracture in breast augmentation and reconstructive surgery and inhibit or prevent infection in the vicinity of the implant site.

Numerous breast implants are suitable for use in the practice of this invention and can be used for cosmetic and reconstructive purposes. Breast implants may be composed of a flexible soft shell filled with a fluid, such as saline solution, polysiloxane, or silicone gel. For example, the breast implant may be composed of an outer polymeric shell having a cavity filled with a plurality of hollow bodies of elastically deformable material containing a liquid saline solution. See, e.g., U.S. Pat. No. 6,099,565. The breast implant may be composed of an envelope of vulcanized silicone rubber that forms a hollow sealed water impermeable shell containing an aqueous solution of polyethylene glycol. See, e.g., U.S. Pat. No. 6,312,466. The breast implant may be composed of an envelope made from a flexible non-absorbable material and a filler material that is a shortening composition (e.g., vegetable oil). See, e.g., U.S. Pat. No. 6,156,066. The breast implant may be composed of a soft, flexible outer membrane and a partially-deformable elastic filler material that is supported by a compartmental internal structure. See, e.g., U.S. Pat. No. 5,961,552. The breast implant may be composed of a non-biodegradable conical shell filled with layers of monofilament yarns formed into resiliently compressible fabric. See, e.g., U.S. Pat. No. 6,432,138. The breast implant may be composed of a shell containing sterile continuous filler material made of continuous yarn of polyolefin or polypropylene. See, e.g., U.S. Pat. No. 6,544,287. The breast implant may be composed of an envelope containing a keratin hydrogel. See, e.g., U.S. Pat. No. 6,371,984. The breast implant may be composed of a hollow, collapsible shell formed from a flexible, stretchable material having a base portion reinforced with a resilient, non-deformable member and a cohesive filler material contained within. See, e.g., U.S. Pat. No. 5,104,409. The breast implant may be composed of a smooth, non-porous, polymeric outer envelope with an affixed non-woven, porous outer layer made of extruded fibers of polycarbonate urethane polymer, which has a soft filler material contained within. See, e.g., U.S. Pat. No. 5,376,117. The breast implant may be configured to be surgically implanted under the pectoral muscle with a second prosthesis implanted between the pectoral muscle and the breast tissue. See, e.g., U.S. Pat. No. 6,464,726. The breast implant may be composed of a homogenous silicone elastomer flexible shell of unitary construction with an interior filling and a rough-textured external surface with randomly formed interconnected cells to promote tissue ingrowth to prevent capsular contracture. See, e.g., U.S. Pat. No. 5,674,285. The breast implant may be a plastic implant with a covering of heparin which is bonded to the surface to prevent or treat capsule formation and/or shrinkage in a blood dry tissue cavity. See, e.g., U.S. Pat. No. 4,713,073. The breast implant may be a sealed, elastic polymer envelope having a microporous structure that is filled with a viscoelastic material (e.g., salt of chondroitin sulfate) to provide a predetermined shape. See, e.g., U.S. Pat. No. 5,344,451.

Breast implants, which may benefit from having the subject polymer composition infiltrated into adjacent tissue according to the present invention, include commercially available products. Commercially available breast implant implants include those from INAMED Corporation (Santa Barbara, Calif.) that sells both Saline-Filled and Silicone-Filled Breast Implants. INAMED's Saline-Filled Breast Implants include the Style 68 Saline Matrix and Style 363LF as well as others in a variety of models, contours, shapes and sizes. INAMED's Silicone-Filled Breast Implants include the Style 10, Style 20 and Style 40 as well as others in a variety of shapes, contours and sizes. INAMED also sells breast tissue expanders, such as the INAMED Style 133 V series tissue expanders, which are used to encourage rapid tissue adherence to maximize expander immobility. Mentor Corporation (Santa Barbara, Calif.) sells the saline-filled Contour Profile Style Breast Implant (available in a variety of models, shapes, contours and sizes) and the SPECTRUM Postoperatively Adjustable Breast Implant that allows adjustment of breast size by adding or removing saline with a simple office procedure for six months post-surgery. Mentor also produces the Contour Profile® Gel (silicone) breast implant in a variety of models, shapes, contours and sizes. Breast implants such as these may benefit from release of a therapeutic agent able to reduce scarring at the implant-tissue interface to minimize the incidence of fibrous contracture. Breast implants such as these may also benefit from release of a therapeutic agent able to prevent or inhibit infection in the vicinity of the implant site.

As described above, implant malposition (movement or migration of the implant after placement) can lead to a variety of complications such as asymmetry and movement below the inframammary crease, and is a leading cause of patient dissatisfaction and revision surgery. In one embodiment the breast implant is coated on the inferior surface (i.e., the surface facing the pectoralis muscle for subglandular breast implants or the surface facing the chest wall for subpectoral breast implants) with a fibrosis-promoting agent or composition, and the coated on the other surfaces (i.e., the surfaces facing the mammary tissue for subglandular breast implants or the surfaces facing the pectoralis muscle for subpectoral breast implants) with an agent or composition that inhibits fibrosis. Such coating may be done directly or by infiltration of the subject polymer composition containing the desired agent into the tissue adjacent to the desired surface, or any combination thereof. This embodiment has the advantage of encouraging fibrosis and fixation of the breast implant into the anatomical location into which it was placed (i.e., to affix the breast implant into the subglandular or subpectoral space preventing implant migration), while preventing the complications associated with encapsulation on the superficial aspects of the breast implant. Representative examples of agents that promote fibrosis and are suitable for delivery from the inferior (deep) surface of the breast implant include silk, wool, silica, bleomycin, neomycin, talcum powder, metallic beryllium, calcium phosphate, calcium sulfate, calcium carbonate, hydroxyapatite, copper, cytokines (e.g., wherein the cytokine is selected from the group consisting of bone morphogenic proteins, demineralized bone matrix, TGFβ, PDGF, VEGF, bFGF, TNFα, NGF, GM-CSF, IGF-1, IL-1-β, IL-8, IL-6, and growth hormone), agents that stimulate cell proliferation (e.g., wherein the agent that stimulates cell proliferation is selected from the group consisting of dexamethasone, isotretinoin, 17-β-estradiol, estradiol, 1-α-25 dihydroxyvitamin D₃, diethylstibesterol, cyclosporine A, N(omega-nitro-L-arginine methyl ester (N(omega-nitro-L-arginine methyl ester)), and all-trans retinoic acid (ATRA)); as well as analogues and derivatives thereof. As an alternative to, or in addition to, coating the inferior surface of the breast implant with the subject polymer composition that contains a fibrosis-promoting agent, a composition that includes a fibrosis-inducing agent can be infiltrated into the space (the base of the surgically created pocket) where the breast implant will be apposed to the underlying tissue.

In one aspect, the present invention provides breast implants having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with breast implants have been described above.

Polymeric compositions may be infiltrated around implanted breast implants by applying the composition directly and/or indirectly into and/or onto (a) tissue adjacent to the breast implant; (b) the vicinity of the breast implant-tissue interface; (c) the region around the breast implant; and (d) tissue surrounding the breast implant. Methods for infiltrating the subject polymer compositions into tissue adjacent to a breast implant include delivering the polymer composition: (a) to the surface of the breast implant (e.g., as an injectable, paste, gel or mesh) during the implantation procedure; (b) to the surface of the tissue (e.g., as an injectable, paste, gel, in situ forming gel or mesh) immediately prior to, or during, implantation of the breast implant; (c) to the surface of the breast implant and/or the tissue surrounding the implanted breast implant (e.g., as an injectable, paste, gel, in situ forming gel or mesh) immediately after the implantation of the breast implant; (d) by topical application of the composition into the anatomical space (e.g., the surgically created pocket) where the breast implant may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the implant may be inserted); (e) via percutaneous injection into the tissue surrounding the breast implant as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the implant.

According to one aspect, any fibrosis-inhibiting and/or anti-infective agent described above may be utilized in the practice of the present invention. In one aspect of the invention, the subject polymer compositions infiltrated into tissue adjacent to breast implants may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As breast implants are made in a variety of configurations and sizes, the exact dose administered will also vary with implant size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the implant, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of implant or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the implant, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of implant or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm²to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

(2) Facial Implants

In one aspect, the subject polymer compositions may be infiltrated into tissue adjacent to a facial implant. The subject polymer compositions may contain a therapeutic agent (e.g., an anti-scarring and/or anti-infective agent).

The soft tissue implant may be a facial implant, including implants for the malar-midface region or submalar region (e.g., cheek implant). Malar and submalar augmentation is often conducted when obvious changes have occurred associated with aging (e.g., hollowing of the cheeks and ptosis of the midfacial soft tissue), midface hypoplasia (a dish-face deformity), post-traumatic and post-tumor resection deformities, and mild hemifacial microsomia. Malar and submalar augmentation may also be conducted for cosmetic purposes to provide a dramatic high and sharp cheek contour. Placement of a malar-submalar implant often enhances the result of a rhytidectomy or rhinoplasty by further improving facial balance and harmony.

There are numerous facial implants that can be used for cosmetic and reconstructive purposes. For example, the facial implant may be a thin teardrop-shaped profile with a broad head and a tapered narrow tail for the mid-facial or submalar region of the face to restore and soften the fullness of the cheeks. See, e.g., U.S. Pat. No. 4,969,901. The facial implant may be composed of a flexible material having a generally concave-curved lower surface and a convex-curved upper surface, which is used to augment the submalar region. See, e.g., U.S. Pat. No. 5,421,831. The facial implant may be a modular prosthesis composed of a thin planar shell and shims that provide the desired contour to the overlying tissue. See, e.g., U.S. Pat. No. 5,514,179. The facial implant may be composed of moldable silicone having a grid of horizontal and vertical grooves on a concave bone-facing rear surface to facilitate tissue ingrowth. See, e.g., U.S. Pat. No. 5,876,447. The facial implant may be composed of a closed-cell, cross-linked, polyethylene foam that is formed into a shell and of a shape to closely conform to the face of a human. See, e.g., U.S. Pat. No. 4,920,580. The facial implant may be a means of harvesting a dermis plug from the skin of the donor after applying a laser beam for ablating the epidermal layer of the skin thereby exposing the dermis and then inserting this dermis plug at a site of facial skin depression. See, e.g., U.S. Pat. No. 5,817,090. The facial implant may be composed of silicone-elastomer with an open-cell structure whereby the silicone elastomer is applied to the surface as a solid before the layer is cured. See, e.g., U.S. Pat. No. 5,007,929. The facial implant may be a hollow perforate mandibular or maxillary dental implant composed of a trans osseous bolt receptor which are secured against the alveolar ridge by contiguous straps. See, e.g., U.S. Pat. No. 4,828,492.

Facial implants, which may benefit from having the subject polymer composition infiltrated into adjacent tissue according to the present invention, include commercially available products. Commercially available facial implants suitable for the practice of this invention include: Tissue Technologies, Inc. (San Francisco, Calif.) sells the ULTRASOFT-RC Facial Implant which is made of soft, pliable synthetic e-PTFE used for soft tissue augmentation of the face. Tissue Technologies, Inc. also sells the ULTRASOFT which is made of tubular e-PTFE indicated for soft tissue augmentation of the facial area and is particularly well suited for use in the lip border and the nasolabial folds. A variety of facial implants are available from ImplanTech Associates including the BINDER SUBMALAR facial implant, the BINDER SUBMALAR II FACIAL IMPLANT, the TERINO MALAR SHELL, the COMBINED SUBMALAR SHELL, the FLOWERS TEAR TROUGH implant; solid silicone facial and malar implants from Allied Biomedical; the Subcutaneous Augmentation Material (S.A.M.), made from microporous ePTFE which supports rapid tissue incorporation and preformed TRIMENSIONAL 3-D Implants from W. L. Gore & Associates, Inc.

Facial implants such as these may benefit from release of a therapeutic agent able to reduce scarring at the implant-tissue interface to minimize the occurrence of fibrous contracture. Facial implants such as these may also benefit from release of a therapeutic agent able to prevent or inhibit infection in the vicinity of the implant site. Infiltration of the subject polymer composition comprising an anti-scarring agent and/or anti-infective agent into tissue adjacent to where the facial implant is or will be implanted may minimize or prevent fibrous contracture in response to facial implants that are placed in the face for cosmetic or reconstructive purposes and/or may inhibit or prevent infection in the vicinity of the implant site. The fibrosis-inhibiting agent may reduce the incidence of capsular contracture, asymmetry, skin dimpling, hardness and repeat surgical interventions (e.g., capsulotomy, capsulectomy, revisions, and removal) and improve patient satisfaction with the procedure.

Regardless of the specific design features, for a facial implant to be effective in cosmetic or reconstructive procedures, the implant must be accurately positioned within the body. Facial implants can migrate following surgery and it is important to achieve attachment of the implant to the underlying periosteum and bone tissue. Facial implants have been described that have a grid of horizontal and vertical grooves on a concave bone-facing rear surface to facilitate tissue ingrowth. Facial implant malposition (movement or migration of the implant after placement) can lead to asymmetry and is a leading cause of patient dissatisfaction and revision surgery. In one embodiment the facial implant is coated on the inferior surface (i.e., the surface facing the periosteum and bone) with a fibrosis-inducing agent or composition, and coated on the other surfaces (i.e., the surfaces facing the skin and subcutaneous tissues) with an agent or composition that inhibits fibrosis. Such coating may be done directly or by infiltration of the subject polymer composition containing the desired agent into the tissue adjacent to the desired surface, or any combination thereof. This embodiment has the advantage of encouraging fibrosis and fixation of the facial implant into the anatomical location into which it was placed (i.e., to affix the facial implant to the underlying bone preventing implant migration), while preventing the complications associated with encapsulation on the superficial aspects of the implant. Representative examples of agents that promote fibrosis and are suitable for delivery from the inferior (deep) surface of the facial implant include silk, wool, silica, bleomycin, neomycin, talcum powder, metallic beryllium, calcium phosphate, calcium sulfate, calcium carbonate, hydroxyapatite, copper, cytokines (e.g., wherein the cytokine is selected from the group consisting of bone morphogenic proteins, demineralized bone matrix, TGFP, PDGF, VEGF, bFGF, TNFα, NGF, GM-CSF, IGF-1, IL-1-β, IL-8, IL-6, and growth hormone), agents that stimulate cell proliferation (e.g., wherein the agent that stimulates cell proliferation is selected from the group consisting of dexamethasone, isotretinoin, 17-β-estradiol, estradiol, 1-α-25 dihydroxyvitamin D₃, diethylstibesterol, cyclosporine A, N(omega-nitro-L-arginine methyl ester), and all-trans retinoic acid (ATRA)); as well as analogues and derivatives thereof. As an alternative to, or in addition to, coating the inferior surface of the facial implant with a composition that contains a fibrosis-promoting agent, the subject polymer composition that includes a fibrosis-inducing agent can be infiltrated into tissue adjacent to the surface or space (e.g., the surface of the periosteum) where the facial implant will be apposed to the underlying tissue.

In one aspect, the present invention provides facial implants having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with facial implants have been described above.

Polymeric compositions may be infiltrated around implanted facial implants by applying the composition directly and/or indirectly into and/or onto (a) tissue adjacent to the facial implant; (b) the vicinity of the facial implant-tissue interface; (c) the region around the facial implant; and (d) tissue surrounding the facial implant. Methods for infiltrating the subject polymer compositions into tissue adjacent to a facial implant include delivering the polymer composition: (a) to the surface of the facial implant (e.g., as an injectable, paste, gel or mesh) during the implantation procedure; (b) to the surface of the tissue (e.g., as an injectable, paste, gel, in situ forming gel or mesh) immediately prior to, or during, implantation of the facial implant; (c) to the surface of the facial implant and/or the tissue surrounding the implanted facial implant (e.g., as an injectable, paste, gel, in situ forming gel or mesh) immediately after the implantation of the facial implant; (d) by topical application of the composition into the anatomical space where the facial implant may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the implant may be inserted); (e) via percutaneous injection into the tissue surrounding the facial implant as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the implant.

According to one aspect, any fibrosis-inhibiting and/or anti-infective agent described above may be utilized in the practice of the present invention. In one aspect of the invention, the subject polymer compositions infiltrated into tissue adjacent to facial implants may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As facial implants are made in a variety of configurations and sizes, the exact dose administered will also vary with implant size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the implant, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of implant or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the implant, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of implant or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

(3) Chin and Mandibular Implants

In one aspect, the subject polymer compositions may be infiltrated into tissue adjacent to a chin or mandibular implant. The subject polymer compositions may contain a therapeutic agent (e.g., an anti-scarring and/or anti-infective agent). Infiltration of the subject polymer compositions into tissue adjacent to the implant site may minimize or prevent fibrous contracture in response to implants placed for cosmetic or reconstructive purposes.

Numerous chin and mandibular implants can be used for cosmetic and reconstructive purposes. For example, the chin implant may be a solid, crescent-shaped implant tapering bilaterally to form respective tails and having a curved projection surface positioned on the outer mandible surface to create a natural chin profile and form a build-up of the jaw. See, e.g., U.S. Pat. No. 4,344,191. The chin implant may be a solid crescent with an axis of symmetry of forty-five degrees, which has a softer, lower durometer material at the point of the chin to simulate the fat pad. See, e.g., U.S. Pat. No. 5,195,951. The chin implant may have a concave posterior surface to cooperate with the irregular bony surface of the mandible and a convex anterior surface with a protuberance for augmenting and providing a natural chin contour. See, e.g., U.S. Pat. No. 4,990,160. The chin implant may have a porous convex surface made of polytetrafluoroethylene having void spaces of size adequate to allow soft tissue ingrowth, while the concave surface made of silicone is nonporous to substantially prevent ingrowth of bony tissue. See, e.g., U.S. Pat. No. 6,277,150.

Chin or mandibular implants, which may benefit from having the subject polymer composition infiltrated into adjacent tissue according to the present invention, include commercially available products. Examples of commercially available chin or mandibular implants include: the TERINO EXTENDED ANATOMICAL chin implant, the GLASGOLD WAFER, the FLOWERS MANDIBULAR GLOVE, MITTELMAN PRE JOWL-CHIN, GLASGOLD WAFER implants, as well as other models from ImplantTech Associates; and the solid silicone chin implants from Allied Biomedical.

Infiltration of the subject polymer composition comprising an anti-scarring agent and/or anti-infective agent into tissue adjacent to where the chin or mandibular implant is or will be implanted may reduce scarring at the implant-tissue interface to minimize the occurrence of fibrous contracture and/or may inhibit or prevent infection in the vicinity of the implant site. Infiltration of the subject polymer composition into tissue adjacent to the chin or mandibular implant site may minimize or prevent fibrous contracture in response to implants that are placed in the chin or mandible for cosmetic or reconstructive purposes. The fibrosis-inhibiting agent can reduce the incidence of capsular contracture, asymmetry, skin dimpling, hardness and repeat surgical interventions (e.g., capsulotomy, capsulectomy, revisions, and removal) and improve patient satisfaction with the procedure.

Regardless of the specific design features, for a chin or mandibular implant to be effective in cosmetic or reconstructive procedures, the implant must be accurately positioned on the face. Chin or mandibular implants can migrate following surgery and it is important to achieve attachment of the implant to the underlying periosteum and bone tissue. Chin or mandibular implant malposition (movement or migration of the implant after placement) can lead to asymmetry and is a leading cause of patient dissatisfaction and revision surgery. In one embodiment the chin or mandibular implant is coated on the inferior surface (i.e., the surface facing the periosteum and the mandible) with a fibrosis-inducing agent or composition, and coated on the other surfaces (i.e., the surfaces facing the skin and subcutaneous tissues) with an agent or composition that inhibits fibrosis. Such coating may be done directly or by infiltration of the subject polymer composition containing the desired agent into the tissue adjacent to the desired surface, or any combination thereof . This embodiment has the advantage of encouraging fibrosis and fixation of the chin or mandibular implant to the underlying mandible (i.e., to affix the implant to the underlying mandible preventing implant migration), while preventing the complications associated with encapsulation on the superficial aspects of the implant. Representative examples of agents that promote fibrosis and are suitable for delivery from the inferior (deep) surface of the chin or mandibular implant include silk, wool, silica, bleomycin, neomycin, talcum powder, metallic beryllium, calcium phosphate, calcium sulfate, calcium carbonate, hydroxyapatite, copper, inflammatory cytokines (e.g., wherein the inflammatory cytokine is selected from the group consisting of bone morphogenic proteins, demineralized bone matrix, TGFP, PDGF, VEGF, bFGF, TNFα, NGF, GM-CSF, IGF-1, IL-1-β, IL-8, IL-6, and growth hormone), agents that stimulate cell proliferation (e.g., wherein the agent that stimulates cell proliferation is selected from the group consisting of dexamethasone, isotretinoin, 17-β-estradiol, estradiol, 1-α-25 dihydroxyvitamin D₃, diethylstibesterol, cyclosporine A, N(omega-nitro-L-arginine methyl ester), and all-trans retinoic acid (ATRA)); as well as analogues and derivatives thereof. As an alternative to, or in addition to, coating the inferior surface of the chin or mandibular implant with a composition that contains a fibrosis-inducing agent, the subject polymer composition that includes a fibrosis-inducing agent can be infiltrated into tissue adjacent to the surface or space (e.g., the surface of the periosteum) where the implant will be apposed to the underlying tissue.

In one aspect, the present invention provides chin or mandibular implants having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with chin or mandibular implants have been described above.

Polymeric compositions may be infiltrated around implanted chin or mandibular implants by applying the composition directly and/or indirectly into and/or onto (a) tissue adjacent to the chin or mandibular implant; (b) the vicinity of the chin or mandibular implant-tissue interface; (c) the region around the chin or mandibular implant; and (d) tissue surrounding the chin or mandibular implant. Methods for infiltrating the subject polymer compositions into tissue adjacent to a chin or mandibular implant include delivering the polymer composition: (a) to the surface of the chin or mandibular implant (e.g., as an injectable, paste, gel or mesh) during the implantation procedure; (b) to the surface of the tissue (e.g., as an injectable, paste, gel, in situ forming gel or mesh) immediately prior to, or during, implantation of the chin or mandibular implant; (c) to the surface of the chin or mandibular implant and/or the tissue surrounding the implanted chin or mandibular implant (e.g., as an injectable, paste, gel, in situ forming gel or mesh) immediately after the implantation of the chin or mandibular implant; (d) by topical application of the composition into the anatomical space where the chin or mandibular implant may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the implant may be inserted); (e) via percutaneous injection into the tissue surrounding the chin or mandibular implant as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the implant.

According to one aspect, any fibrosis-inhibiting and/or anti-infective agent described above may be utilized in the practice of the present invention. In one aspect of the invention, the subject polymer compositions infiltrated into tissue adjacent to chin or mandibular implants may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As chin or mandibular implants are made in a variety of configurations and sizes, the exact dose administered will also vary with implant size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the implant, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of implant or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the implant, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of implant or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

(4) Nasal Implants

In one aspect, the subject polymer compositions may be infiltrated into tissue adjacent to a nasal implant. The subject polymer compositions may contain a therapeutic agent (e.g., an anti-scarring and/or anti-infective agent). Infiltration of the subject polymer compositions into tissue adjacent to the implant site may minimize or prevent fibrous contracture in response to implants placed for cosmetic or reconstructive purposes.

Numerous nasal implants are suitable for the practice of this invention that can be used for cosmetic and reconstructive purposes. For example, the nasal implant may be elongated and contoured with a concave surface on a selected side to define a dorsal support end that is adapted to be positioned over the nasal dorsum to augment the frontal and profile views of the nose. See, e.g., U.S. Pat. No. 5,112,353. The nasal implant may be composed of substantially hard-grade silicone configured in the form of an hourglass with soft silicone at the tip. See, e.g., U.S. Pat. No. 5,030,232. The nasal implant may be composed of essentially a principal component being an aryl acrylic hydrophobic monomer with the remainder of the material being a cross-linking monomer and optionally one or more additional components selected from the group consisting of UV-light absorbing compounds and blue-light absorbing compounds. See, e.g., U.S. Pat. No. 6,528,602. The nasal implant may be composed of a hydrophilic synthetic cartilaginous material with pores of controlled size randomly distributed throughout the body for replacement of fibrous tissue. See, e.g., U.S. Pat. No. 4,912,141.

Nasal implants, which may benefit from having the subject polymer composition infiltrated into adjacent tissue according to the present invention, include commercially available products. Examples of commercially available nasal implants suitable for use in the practice of this invention include the FLOWERS DORSAL, RIZZO DORSAL, SHIRAKABE, and DORSAL COLUMELLA nasal implants from ImplantTech Associates and solid silicone nasal implants from Allied Biomedical.

Nasal implants such as these may benefit from release of a therapeutic agent able to reduce scarring at the implant-tissue interface to minimize the occurrence of fibrous contracture. Nasal implants such as these may also benefit from release of a therapeutic agent able to prevent or inhibit infection in the vicinity of the implant site. Infiltration of the subject polymer composition comprising an anti-scarring agent and/or anti-infective agent into tissue adjacent to where the nasal implant is or will be implanted may minimize or prevent fibrous contracture in response to implants that are placed in the nose for cosmetic or reconstructive purposes. The fibrosis-inhibiting agent may reduce the incidence of capsular contracture, asymmetry, skin dimpling, hardness and repeat surgical interventions (e.g., capsulotomy, capsulectomy, revisions, and removal) and improve patient satisfaction with the procedure.

Regardless of the specific design features, for a nasal implant to be effective in cosmetic or reconstructive procedures, the implant must be accurately positioned on the face. Nasal implants can migrate following surgery and it is important to achieve attachment of the implant to the underlying cartilage and/or bone tissue in the nose. Nasal implant malposition (movement or migration of the implant after placement) can lead to asymmetry and is a leading cause of patient dissatisfaction and revision surgery. In one embodiment the nasal implant is coated on the inferior surface (i.e., the surface facing the nasal cartilage and/or bone) with a fibrosis-inducing agent or composition, and coated on the other surfaces (i.e., the surfaces facing the skin and subcutaneous tissues) with an agent or composition that inhibits fibrosis. Such coating may be done directly or by infiltration of the subject polymer composition containing the desired agent into the tissue adjacent to the desired surface, or any combination thereof. This embodiment has the advantage of encouraging fibrosis and fixation of the nasal implant to the underlying nasal cartilage or bone (i.e., to affix the implant to the underlying cartilage or bone of the nose). preventing implant migration), while preventing the complications associated with encapsulation on the superficial aspects of the implant. Representative examples of agents that promote fibrosis and are suitable for delivery from the inferior (deep) surface of the nasal implant include silk, wool, silica, bleomycin, neomycin, talcum powder, metallic beryllium, calcium phosphate, calcium sulfate, calcium carbonate, hydroxyapatite, copper, inflammatory cytokines (e.g., wherein the inflammatory cytokine is selected from the group consisting of bone morphogenic proteins, demineralized bone matrix, TGFβ, PDGF, VEGF, bFGF, TNFα, NGF, GM-CSF, IGF-1, IL-1-β, IL-8, IL-6, and growth hormone), agents that stimulate cell proliferation (e.g., wherein the agent that stimulates cell proliferation is selected from the group consisting of dexamethasone, isotretinoin, 17-β-estradiol, estradiol, 1-α-25 dihydroxyvitamin D₃, diethylstibesterol, cyclosporine A, N(omega-nitro-L-arginine methyl ester), and all-trans retinoic acid (ATRA)); as well as analogues and derivatives thereof. As an alternative to, or in addition to, coating the inferior surface of the nasal implant with the subject polymer composition that contains a fibrosis-inducing agent, a composition that includes a fibrosis-inducing agent can be infiltrated into tissue adjacent to the surface or space (e.g., the surface of the nasal cartilage or bone) where the implant will be apposed to the underlying tissue.

In one aspect, the present invention provides nasal implants having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with nasal implants have been described above.

Polymeric compositions may be infiltrated around implanted nasal implants by applying the composition directly and/or indirectly into and/or onto (a) tissue adjacent to the nasal implant; (b) the vicinity of the nasal implant-tissue interface; (c) the region around the nasal implant; and (d) tissue surrounding the nasal implant. Methods for infiltrating the subject polymer compositions into tissue adjacent to a nasal implant include delivering the polymer composition: (a) to the surface of the nasal implant (e.g., as an injectable, paste, gel or mesh) during the implantation procedure; (b) to the surface of the tissue (e.g., as an injectable, paste, gel, in situ forming gel or mesh) immediately prior to, or during, implantation of the nasal implant; (c) to the surface of the nasal implant and/or the tissue surrounding the implanted nasal implant (e.g., as an injectable, paste, gel, in situ forming gel or mesh) immediately after the implantation of the nasal implant; (d) by topical application of the composition into the anatomical space where the nasal implant may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the implant may be inserted); (e) via percutaneous injection into the tissue surrounding the nasal implant as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the implant.

According to one aspect, any fibrosis-inhibiting and/or anti-infective agent described above may be utilized in the practice of the present invention. In one aspect of the invention, the subject polymer compositions infiltrated into tissue adjacent to nasal implants may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As nasal implants are made in a variety of configurations and sizes, the exact dose administered will also vary with implant size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the implant, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of implant or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the implant, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of implant or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

(5) Lip Implants

In one aspect, the subject polymer compositions may be infiltrated into tissue adjacent to a lip implant. The subject polymer compositions may contain a therapeutic agent (e.g., an anti-scarring and/or anti-infective agent). Infiltration of the subject polymer compositions into tissue adjacent to the implant site may minimize or prevent fibrous contracture in response to implants placed for cosmetic or reconstructive purposes.

There are numerous lip implants that can be used for cosmetic and reconstructive purposes. For example, the lip implant may be composed of non-biodegradable expanded, fibrillated polytetrafluoroethylene having an interior cavity extending longitudinally whereby fibrous tissue ingrowth may occur to provide soft tissue augmentation. See, e.g., U.S. Pat. Nos. 5,941,910 and 5,607,477. The lip implant may comprise soft, malleable, elastic, non-resorbing prosthetic particles that have a rough, irregular surface texture, which are dispersed in a non-retentive compatible physiological vehicle. See, e.g., U.S. Pat. No. 5,571,182.

Lip implants, which may benefit from having the subject polymer composition infiltrated into adjacent tissue according to the present invention, include commercially available products. Commercially available lip implants suitable for use in the present invention include SOFTFORM from Tissue Technologies, Inc. (San Francisco, Calif.), which has a tube-shaped design made of synthetic ePTFE; ALLODERM sheets (Allograft Dermal Matrix Grafts), which are sold by LifeCell Corporation (Branchburg, N.J.) may also be used as an implant to augment the lip. ALLODERM sheets are very soft and easily augment the lip in a diffuse manner. W.L. Gore and Associates (Newark, Del.) sells solid implantable threads that may also be used for lip implants.

Infiltration of the subject polymer composition comprising an anti-scarring agent and/or anti-infective agent into tissue adjacent to where the lip implant is or will be implanted may reduce scarring at the implant-tissue interface to minimize the occurrence of fibrous contracture and/or may inhibit or prevent infection in the vicinity of the implant site. Infiltration of the subject polymer composition into tissue adjacent to the lip implant site may minimize or prevent fibrous contracture in response to implants that are placed in the lips for cosmetic or reconstructive purposes. The fibrosis-inhibiting agent can reduce the incidence of asymmetry, skin dimpling, hardness and repeat interventions and improve patient satisfaction with the procedure.

In one embodiment of the invention, the lip implant is coated on one aspect with a composition that inhibits fibrosis, as well as being coated with a composition or compound that promotes fibrous tissue ingrowth on another aspect. Such coating may be done directly or by infiltration of the subject polymer composition containing the desired agent into the tissue adjacent to the desired surface, or any combination thereof. This embodiment has the advantage of encouraging fibrosis and fixation of the lip implant to the adjacent tissues, while preventing the complications associated with fibrous encapsulation on the superficial aspects of the implant. Representative examples of agents that promote fibrosis and are suitable for delivery from the inferior (deep) surface of the lip implant include silk, wool, silica, bleomycin, neomycin, talcum powder, metallic beryllium, calcium phosphate, calcium sulfate, calcium carbonate, hydroxyapatite, copper, inflammatory cytokines (e.g., wherein the inflammatory cytokine is selected from the group consisting of bone morphogenic proteins, demineralized bone matrix, TGFβ, PDGF, VEGF, bFGF, TNFα, NGF, GM-CSF, IGF-1, IL-1-β, IL-8, IL-6, and growth hormone), agents that stimulate cell proliferation (e.g., wherein the agent that stimulates cell proliferation is selected from the group consisting of dexamethasone, isotretinoin, 17-β-estradiol, estradiol, 1-α-25 dihydroxyvitamin D₃, diethylstibesterol, cyclosporine A, N(omega-nitro-L-arginine methyl ester), and all-trans retinoic acid (ATRA)); as well as analogues and derivatives thereof. As an alternative to, or in addition to, coating the inferior surface of the lip implant with a composition that contains a fibrosis-inducing agent, the subject polymer composition that includes a fibrosis-inducing agent can be injected directly into the lip where the implant will be placed.

In one aspect, the present invention provides lip implants having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with lip implants have been described above.

Polymeric compositions may be infiltrated around implanted lip implants by applying the composition directly and/or indirectly into and/or onto (a) tissue adjacent to the lip implant; (b) the vicinity of the lip implant-tissue interface; (c) the region around the lip implant; and (d) tissue surrounding the lip implant. Methods for infiltrating the subject polymer compositions into tissue adjacent to a lip implant include delivering the polymer composition: (a) to the surface of the lip implant (e.g., as an injectable, paste, gel or mesh) during the implantation procedure; (b) to the surface of the tissue (e.g., as an injectable, paste, gel, in situ forming gel or mesh) immediately prior to, or during, implantation of the lip implant; (c) to the surface of the lip implant and/or the tissue surrounding the implanted lip implant (e.g., as an injectable, paste, gel, in situ forming gel or mesh) immediately after the implantation of the lip implant; (d) by topical application of the composition into the anatomical space where the lip implant may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the implant may be inserted); (e) via percutaneous injection into the tissue surrounding the lip implant as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the implant.

According to one aspect, any fibrosis-inhibiting and/or anti-infective agent described above may be utilized in the practice of the present invention. In one aspect of the invention, the subject polymer compositions infiltrated into tissue adjacent to lip implants may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As lip implants are made in a variety of configurations and sizes, the exact dose administered will also vary with implant size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the implant, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of implant or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the implant, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of implant or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm²to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

(6) Pectoral Implants

In one aspect, the subject polymer compositions may be infiltrated into tissue adjacent to a pectoral implant. The subject polymer compositions may contain a therapeutic agent (e.g., an anti-scarring and/or anti-infective agent). Infiltration of the subject polymer compositions into tissue adjacent to the implant site may minimize or prevent fibrous contracture in response to implants placed for cosmetic or reconstructive purposes.

There are numerous pectoral implants that can be combined with a fibrosis-inhibiting agent and used for cosmetic and reconstructive purposes. For example, the pectoral implant may be composed of a unitary rectangular body having a slightly concave cross-section that is divided by edges into sections. See, e.g., U.S. Pat. No. 5,112,352. The pectoral implant may be composed of a hollow shell formed of a flexible elastomeric envelope that is filled with a gel or viscous liquid containing polyacrylamide and derivatives of polyacrylamide. See, e.g., U.S. Pat. No. 5,658,329.

Pectoral implants, which may benefit from having the subject polymer composition infiltrated into adjacent tissue according to the present invention, include commercially available products. Commercially available pectoral implants suitable for use in the present invention include solid silicone implants from Allied Biomedical. Pectoral implants such as these may benefit from release of a therapeutic agent able to reduce scarring at the implant-tissue interface to minimize the incidence of fibrous contracture. Pectoral implants such as these may also benefit from release of a therapeutic agent able to prevent or inhibit infection in the vicinity of the implant site.

As described previously, implant malposition (movement or migration of the implant after placement) can lead to a variety of complications such as asymmetry, and is a leading cause of patient dissatisfaction and revision surgery. In one embodiment the pectoral implant is coated on the inferior surface (i.e., the surface facing the chest wall) with a fibrosis-promoting agent or composition, and the coated on the other surfaces (i.e., the surfaces facing the pectoralis muscle) with an agent or composition that inhibits fibrosis. Such coating may be done directly or by infiltration of the subject polymer composition containing the desired agent into the tissue adjacent to the desired surface, or any combination thereof. This embodiment has the advantage of encouraging fibrosis and fixation of the pectoral implant into the anatomical location into which it was placed (i.e., to affix the pectoral implant into the subpectoral space preventing implant migration), while preventing the complications associated with encapsulation on the superficial aspects of the pectoral implant. Representative examples of agents that promote fibrosis and are suitable for delivery from the inferior (deep) surface of the pectoral implant include silk, wool, silica, bleomycin, neomycin, talcum powder, metallic beryllium, calcium phosphate, calcium sulfate, calcium carbonate, hydroxyapatite, copper, cytokines (e.g., wherein the cytokine is selected from the group consisting of bone morphogenic proteins, demineralized bone matrix, TGFβ, PDGF, VEGF, bFGF, TNFα, NGF, GM-CSF, IGF-1, IL-1-β, IL-8, IL-6, and growth hormone), agents that stimulate cell proliferation (e.g., wherein the agent that stimulates cell proliferation is selected from the group consisting of dexamethasone, isotretinoin, 17-β-estradiol, estradiol, 1-α-25 dihydroxyvitamin D₃, diethylstibesterol, cyclosporine A, N(omega-nitro-L-arginine methyl ester), and all-trans retinoic acid (ATRA)); as well as analogues and derivatives thereof. As an alternative to, or in addition to, coating the inferior surface of the pectoral implant with a composition that contains a fibrosis-promoting agent, the subject polymer composition that includes a fibrosis-inducing agent can be infiltrated into tissue adjacent to the space (the base of the surgically created subpectoral pocket) where the pectoral implant will be apposed to the underlying tissue.

In one aspect, the present invention provides pectoral implants having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with pectoral implants have been described above.

Polymeric compositions may be infiltrated around implanted pectoral implants by applying the composition directly and/or indirectly into and/or onto (a) tissue adjacent to the pectoral implant; (b) the vicinity of the pectoral implant-tissue interface; (c) the region around the pectoral implant; and (d) tissue surrounding the pectoral implant. Methods for infiltrating the subject polymer compositions into tissue adjacent to a pectoral implant include delivering the polymer composition: (a) to the surface of the pectoral implant (e.g., as an injectable, paste, gel or mesh) during the implantation procedure; (b) to the surface of the tissue (e.g., as an injectable, paste, gel, in situ forming gel or mesh) immediately prior to, or during, implantation of the pectoral implant; (c) to the surface of the pectoral implant and/or the tissue surrounding the implanted pectoral implant (e.g., as an injectable, paste, gel, in situ forming gel or mesh) immediately after the implantation of the pectoral implant; (d) by topical application of the composition into the anatomical space where the pectoral implant may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the implant may be inserted); (e) via percutaneous injection into the tissue surrounding the pectoral implant as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases-it is understood that the subject polymer compositions may be infiltrated into tissue adjacent to all or a portion of the implant.

According to one aspect, any fibrosis-inhibiting and/or anti-infective agent described above may be utilized in the practice of the present invention. In one aspect of the invention, the subject polymer compositions infiltrated into tissue adjacent to pectoral implants may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As pectoral implants are made in a variety of configurations and sizes, the exact dose administered will also vary with implant size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the implant, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of implant or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the implant, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of implant or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

(7) Autogenous Tissue Implants

In one aspect, the subject polymer compositions may be infiltrated into tissue adjacent to an autogenous tissue implant. The subject polymer compositions may contain a therapeutic agent (e.g., an anti-scarring and/or anti-infective agent). Autogenous tissue implants include, without limitation, adipose tissue, autogenous fat implants, dermal implants, dermal or tissue plugs, muscular tissue flaps and cell extraction implants. Adipose tissue implants may also be known as autogenous fat implants, fat grafting, free fat transfer, autologous fat transfer/transplantation, dermal fat implants, liposculpture, lipostructure, volume restoration, micro-lipoinjection and fat injections.

Autogenous tissue implants have been used for decades for soft tissue augmentation in plastic and reconstructive surgery. Autogenous tissue implants may be used, for example, to enlarge a soft tissue site (e.g., breast or penile augmentation), to minimize facial scarring (e.g., acne scars), to improve facial volume in diseases (e.g., hemifacial atrophy), and to minimize facial aging, such as sunken cheeks and facial lines (e.g., wrinkles). These injectable autogenous tissue implants are biocompatible, versatile, stable, long-lasting and natural-appearing. Autogenous tissue implants involve a simple procedure of removing tissue or cells from one area of the body (e.g., surplus fat cells from abdomen or thighs) and then re-implanted them in another area of the body that requires reconstruction or augmentation. Autogenous tissue is soft and feels natural. Autogenous soft tissue implants may be composed of a variety of connective tissues, including, without limitation, adipose or fat, dermal tissue, fibroblast cells, muscular tissue or other connective tissues and associated cells. An autogenous tissue implant is introduced to correct a variety of deficiencies, it is not immunogenic, and it is readily available and inexpensive.

In one aspect, autogenous tissue implants may be composed of fat or adipose. The extraction and implantation procedure of adipose tissue involves the aspiration of fat from the subcutaneous layer, usually of the abdominal wall by means of a suction syringe, and then injected it into the subcutaneous tissues overlying a depression. Autologous fat is commonly used as filler for depressions of the body surface (e.g., for bodily defects or cosmetic purposes), or it may be used to protect other tissue (e.g., protection of the nerve root following surgery). Fat grafts may also be used for body prominences that require padding of soft tissue to prevent sensitivity to pressure. When fat padding is lacking, the overlying skin may be adherent to the bone, leading to discomfort and even pain, which occurs, for example, when a heel spur or bony projection occurs on the plantar region of the heel bone (also known as the calcaneous). In this case, fat grafting may provide the interposition of the necessary padding between the bone and the skin. U.S. Pat. No. 5,681,561, describes, for example, an autogenous fat graft that includes an anabolic hormone, amino acids, vitamins, and inorganic ions to improve the survival rate of the lipocytes once implanted into the body.

In another aspect, autogenous tissue implants may be composed of pedicle flaps that typically originate from the back (e.g., latissimus dorsi myocutaneous flap) or the abdomen (e.g., transverse rectus abdominus myocutaneous or TRAM flap). Pedicle flaps may also come from the buttocks, thigh or groin. These flaps are detached from the body and then transplanted by reattaching blood vessels using microsurgical procedures. These muscular tissue flaps are most frequently used for post-mastectomy closure and reconstruction. Some other common closure applications for muscular tissue flaps include coverage of defects in the head and neck area, especially defects created from major head and neck cancer resection; additional applications include coverage of chest wall defects other than mastectomy deformities. The latissimus dorsi may also be used as a reverse flap, based upon its lumbar perforators, to close congenital defects of the spine such as spina bifida or meningomyelocele. For example, U.S. Pat. No. 5,765,567 describes methodology of using an autogenous tissue implant in the form of a tissue flap having a cutaneous skin island that may be used for contour correction and enlargement for the reconstruction of breast tissue. The tissue flap may be a free flap or a flap attached via a native vascular pedicle.

In another aspect, the autogenous tissue implant may be a suspension of autologous dermal fibroblasts that may be used to provide cosmetic augmentation. See, e.g., U.S. Pat. Nos. 5,858,390; 5,665,372 and 5,591,444. This U.S. patent describes a method for correcting cosmetic and aesthetic defects in the skin by the injection of a suspension of autologous dermal fibroblasts into the dermis and subcutaneous tissue subadjacent to the defect. Typical defects that can be corrected by this method include rhytids, stretch marks, depressed scars, cutaneous depressions of non-traumatic origin, scaring from acne vulgaris, and hypoplasia of the lip. The fibroblasts that are injected are histocompatible with the subject and have been expanded by passage in a cell culture system for a period of time in protein free medium.

In another aspect, the autogenous tissue implant may be a dermis plug harvested from the skin of the donor after applying a laser beam for ablating the epidermal layer of the skin thereby exposing the dermis and then inserting this dermis plug at a site of facial skin depressions. See, e.g., U.S. Pat. No. 5,817,090. This autogenous tissue implant may be used to treat facial skin depressions, such as acne scar depression and rhytides. Dermal grafts have also been used for correction of cutaneous depressions where the epidermis is removed by dermabrasion.

As is the case for other types of synthetic implants (described above), autogenous tissue implants also have a tendency to migrate, extrude, become infected, or cause painful and deforming capsular contractures. Infiltration of the subject polymer composition comprising a therapeutic agent (e.g., an anti-scarring agent and/or anti-infective agent) into tissue adjacent to where the autogenous tissue implant is or will be implanted may minimize or prevent fibrous contracture in response to autogenous tissue implants that are placed in the body for cosmetic or reconstructive purposes and/or may inhibit or prevent infection in the vicinity of the implant site.

Autogenous tissue implants such as these may benefit from release of a therapeutic agent able to reducing scarring at the implant-tissue interface to minimize fibrous encapsulation. Autogenous tissue implants such as these may also benefit from release of a therapeutic agent able to prevent or inhibit infection in the vicinity of the implant site. In one aspect, the present invention provides autogenous tissue implants having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in connection with autogenous tissue implants have been described above.

Polymeric compositions may be infiltrated around implanted autogenous tissue implants by applying the composition directly and/or indirectly into and/or onto (a) tissue adjacent to the autogenous tissue implant; (b) the vicinity of the autogenous tissue implant-tissue interface; (c) the region around the autogenous tissue implant; and (d) tissue surrounding the autogenous tissue implant. Methods for infiltrating the subject polymer compositions into tissue adjacent to an autogenous tissue implant include delivering the polymer composition: (a) to the surface of the autogenous tissue implant (e.g., as an injectable, paste, gel or mesh) during the implantation procedure; (b) to the surface of the tissue (e.g., as an injectable, paste, gel, in situ forming gel or mesh) immediately prior to, or during, implantation of the autogenous tissue implant; (c) to the surface of the autogenous tissue implant and/or the tissue surrounding the implanted autogenous tissue implant (e.g., as an injectable, paste, gel, in situ forming gel or mesh) immediately after the implantation of the autogenous tissue implant; (d) by topical application of the composition into the anatomical space where the autogenous tissue implant may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the implant may be inserted); (e) via percutaneous injection into the tissue surrounding the autogenous tissue implant as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject polymer compositions may be infiltrated into tissue adjacent to all or a portion of the implant.

According to one aspect, any fibrosis-inhibiting and/or anti-infective agent described above may be utilized in the practice of the present invention. In one aspect of the invention, the subject polymer compositions infiltrated into tissue adjacent to autogenous tissue implants may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As autogenous tissue implants are made in a variety of configurations and sizes, the exact dose administered will also vary with implant size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the polymer composition in effective concentrations in a time period that may be measured from the time of infiltration into tissue adjacent to the implant, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of implant or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the polymer composition in effective concentrations in a time period that may be measured from the time of infiltration into tissue adjacent to the implant, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg. or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of implant or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different polymer compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) may be utilized to enhance the antibacterial activity of the composition.

Although numerous examples of soft tissue implants have been described above, all possess similar design features and cause similar unwanted tissue reactions following implantation and may introduce or promote infection in the area of the implant site. It should be obvious to one of skill in the art that commercial soft tissue implants not specifically cited above as well as next-generation and/or subsequently-developed commercial soft tissue implant products are to be anticipated and are suitable for use under the present invention. The cosmetic implant should be positioned in a very precise manner to ensure that augmentation is achieved correct anatomical location in the body. All, or parts, of a cosmetic implant can migrate following surgery, excessive scar tissue growth can occur around the implant, and/or infection can occur in the vicinity of the implant site, which can lead to a reduction in the performance of these devices. Soft tissue implants having the subject polymer compositions infiltrated into tissue adjacent to the implant-tissue interface can be used to increase the efficacy and/or the duration of activity of the implant. Soft tissue implants may also benefit from release of a therapeutic agent able to prevent or inhibit infection in the vicinity of the implant site. In one aspect, the present invention provides soft tissue implants having the subject polymer compositions infiltrated into adjacent tissue, where the subject polymer compositions may include a therapeutic agent (e.g., an anti-scarring and/or anti-infective agent). Numerous polymeric and non-polymeric delivery systems for use in conjunction with soft tissue implants have been described above. These compositions can further include one or more fibrosis-inhibiting agents such that the overgrowth of granulation or fibrous tissue is inhibited or reduced and/or one or more anti-infective agents such that infection in the vicinity of the implant site is inhibited or prevented.

The present invention, in various aspects and embodiments, provides the following methods for implanting medical devices:

1 . Medical Device

In one aspect, the present invention provides a method for implanting a medical device comprising: (a) infiltrating a tissue of a host where the medical device is to be, or has been, implanted with i) an anti-fibrotic agent, ii) an anti-infective agent, iii) a polymer; iv) a composition comprising an anti-fibrotic agent and a polymer, v) a composition comprising an anti-infective agent and a polymer, or vi) a composition comprising an anti-fibrotic agent, an anti-infective agent and a polymer, and (b) implanting the medical device into the host.

Optionally, in separate aspects, the invention provides: a method for implanting a medical device comprising: (a) infiltrating a tissue of a host where the medical device is to be, or has been, implanted with an anti-fibrotic agent, and (b) implanting the medical device into the host; a method for implanting a medical device comprising: (a) infiltrating a tissue of a host where the medical device is to be, or has been, implanted with an anti-infective agent, and (b) implanting the medical device into the host; a method for implanting a medical device comprising: (a) infiltrating a tissue of a host where the medical device is to be, or has been, implanted with a polymer; and (b) implanting the medical device into the host; a method for implanting a medical device comprising: (a) infiltrating a tissue of a host where the medical device is to be, or has been, implanted with a composition comprising an anti-fibrotic agent and a polymer, and (b) implanting the medical device into the host; a method for implanting a medical device comprising: (a) infiltrating a tissue of a host where the medical device is to be, or has been, implanted with a composition comprising an anti-infective agent and a polymer, and (b) implanting the medical device into the host; and a method for implanting a medical device comprising: (a) infiltrating a tissue of a host where the medical device is to be, or has been, implanted with a composition comprising an anti-fibrotic agent, an anti-infective agent and a polymer, and (b) implanting the medical device into the host.

For each afore stated aspect, one or more (e.g., any two) of the following features may be used to further define the invention in terms of the device used in the inventive method: the device is an intravascular device; the device is a gastrointestinal stent; the device is a tracheal and bronchial stent; the device is a genital urinary stent; the device is an ear and nose stent; the device is an ear ventilation; the device is an intraocular implant; the device is a vascular graft; the device comprises a film or a mesh; the device is a glaucoma drainage device; the device is a prosthetic heart valve or a component thereof; the device is a penile implant; the device is an endotracheal or tracheostomy tube; the device is a peritoneal dialysis catheter; the device is a central nervous system shunt or a pressure monitoring device; the device is an inferior vena cava filter; the device is a gastrointestinal device; the device is a central venous catheter; the device is a ventricular assist device; the device is a spinal implant; the device is an implantable electrical device; the device is an implantable sensor; the device is an implantable pump; and/or the device is a soft tissue implant.

2. Intravascular Device

In another aspect, the present invention provides a method for implanting a medical device comprising: (a) infiltrating a tissue of a host where the medical device is to be, or has been, implanted with i) an anti-fibrotic agent, ii) an anti-infective agent, iii) a polymer; iv) a composition comprising an anti-fibrotic agent and a polymer, v) a composition comprising an anti-infective agent and a polymer, or vi) a composition comprising an anti-fibrotic agent, an anti-infective agent and a polymer, and (b) implanting the medical device into the host, where the medical device is an intravascular device.

Optionally, in separate aspects, the invention provides: a method for implanting a medical device comprising: (a) infiltrating a tissue of a host where the medical device is to be, or has been, implanted with an anti-fibrotic agent, and (b) implanting the medical device into the host; a method for implanting a medical device comprising: (a) infiltrating a tissue of a host where the medical device is to be, or has been, implanted with an anti-infective agent, and (b) implanting the medical device into the host; a method for implanting a medical device comprising: (a) infiltrating a tissue of a host where the medical device is to be, or has been, implanted with a polymer; and (b) implanting the medical device into the host; a method for implanting a medical device comprising: (a) infiltrating a tissue of a host where the medical device is to be, or has been, implanted with a composition comprising an anti-fibrotic agent and a polymer, and (b) implanting the medical device into the host; a method for implanting a medical device comprising: (a) infiltrating a tissue of a host where the medical device is to be, or has been, implanted with a composition comprising an anti-infective agent and a polymer, and (b) implanting the medical device into the host; and a method for implanting a medical device comprising: (a) infiltrating a tissue of a host where the medical device is to be, or has been, implanted with a composition comprising an anti-fibrotic agent, an anti-infective agent and a polymer, and (b) implanting the medical device into the host.

For each afore stated aspect, one or more (e.g., any two) of the following features may be used to further define the invention in terms of the device used in the inventive method: the medical device is a catheter; the medical device is a balloon catheter; the medical device is a balloon; the medical device is a stent graft; the medical device is a guidewire; the medical device is a stent; the medical device is an intravascular stent; the medical device is a metallic stent; the medical device is a polymeric stent; the medical device is a biodegradable stent; the medical device is a non-biodegradable stent; the medical device is a self expandable stent; the medical device is a balloon expandable stent; the medical device is a covered stent; the medical device is a drug eluting stent; the medical device is a stent that comprises a radio-opaque material; the medical device is a stent that comprises an echogenic material; the medical device is a stent that comprise an MRI responsive material; the medical device is an anastomotic connector device; the medical device is an artery to artery anastomotic connector device; the medical device is a vein to artery anastomotic connector device; the medical device is an artery to vein anastomotic connector device; the medical device is an artery to synthetic graft anastomotic connector device; the medical device is a synthetic graft to artery anastomotic conncector device; the medical device is a vein to synthetic graft anastomotic connector device; the medical device is a synthetic graft to vein anastomotic connector device; the medical device is a vascular clip; the medical device is a vascular suture; the medical device is a vascular clamp; the medical device is a suturing device; the medical device is an anastomotic coupler; the medical device is an automated or modified suture device; the medical device is a micromehical anastomotic connector device; the medical device is an anastomotic coupling device that facilitates automated attachement of a graft or vessel to an aperture or orifice in a target vessel without the use of sutures or staples; the medical device is an anastomotic coupling device that comprises a tubular graft conduit and may be placed in a side wall of a target vessel so that the tubular graft conduit may be extended from the target vessel; the medical device is an anastomotic coupler in the form of a frame; the medical device is an anastomotic coupler in a ring-like form; the medical device is a resorbable anastomotic coupler; the medical device is an anastomotic coupler that comprises a bioabsorbable and elastomeric material; the medical device is an anastomotic coupler adapted to connect a first blood vessel with a second blood vessel with a graft vessel; the medical device is an anastomotic coupler adapted to connect a first blood vessel with a second blood vessel without a graft vessel; the medical device is an anastomotic coupler that is incorporated in the design of a vascular graft; the medical device is an anastomotic coupler that comprises a graft that incorporates a fixation mechanism; the medical device is an anastomotic coupler that comprises a compressible, expandable fitting for securing the ends of a bypass graft to two vessels; the medical device is an anastomotic coupler that comprises a pair of coupling disc members for joining two vessels in an end to end or end to side fashion; the medical device is a proximal aortic connector; the medical device is a distal coronary connector; the medical device is a bypass device made of a biocompatible material; the medical device is a bypass device made of at least partially a metal or metal alloy; the medical device is a bypass device made of at least partially a synthetic polymer; the medical device is a bypass device made of at least partially naturally derived polymer; the medical device is a tubular anastomotic coupler that comprises a tubular structure that may be attached directly to a proximal blood vessel; the medical device is a tubular anastomotic coupler that comprises a tubular structure that may be attached directly to a distal blood vessel; the medical device is a tubular anastomotic coupler that has a proximal end attachable to a proximal vessel and a distal end attachable to a bypass graft; the medical device is a tubular anastomotic coupler that has a proximal end attachable to a graft vessel that is secured to a proximal blood vessel and a distal end attachable to a distal blood vessel; the medical device is an anastomotic connector device adapted for end to end anastomosis procedures; the medical device is an anastomotic stent; the medical device is anastomotic sleeve; the medical device is an anastomotic connector device adapted for end to side anastomosis procedures; the medical device is a single lumen bypass device; the medical device is a multi-lumen bypass device; the medical device is an anastomotic coupling device that comprises a single tubular portion that may be used as a shunt to divert blood from a source vessel to a graft vessel; the medical device is anastomotic coupling device that comprises more than one tubular portion, and wherein at least one tubular portion may be used as a shunt for diverting blood between a source vessel and a target vessel; the medical device is an anastomotic connector device that comprises a tubular portion, and wherein one or more ends of the tubular portion may be inserted into the end or into the side of one or more blood vessels; the medical device is a multi-lumen anastomotic connector device that at least one arm of the device may be attached to a graft vessel; the medical device is an anastomotic connector device that includes three or more tubular arms that extend from a junction site; the medical device is a multi-lumen anastomotic connector device is generally T-shaped; the medical device is a multi-lumen anastomotic connector device is generally Y shaped; the medical device is an anastomotic connector device that comprises a tube for bypassing blood flow directly from a portion of the heart to a coronary artery; the medical device is an anastomotic connector device that comprises a network of interconnected tubular conduits; and the medical device is an anastomotic connector device that is configured with two or more termini that provide a vessel interface without the need for sutures and a fluid communication through an intersecting lumen.

3. Gastrointestinal Stent

In another aspect, the present invnention provides a method for implanting a medical device comprising: (a) infiltrating a tissue of a host where the medical device is to be, or has been, implanted with i) an anti-fibrotic agent, ii) an anti-infective agent, iii) a polymer; iv) a composition comprising an anti-fibrotic agent and a polymer, v) a composition comprising an anti-infective agent and a polymer, or vi) a composition comprising an anti-fibrotic agent, an anti-infective agent and a polymer, and (b) implanting the medical device into the host, where the medical device is a gastrointestinal stent.

Optionally, in separate aspects, the invention provides: a method for implanting a medical device comprising: (a) infiltrating a tissue of a host where the medical device is to be, or has been, implanted with an anti-fibrotic agent, and (b) implanting the medical device into the host; a method for implanting a medical device comprising: (a) infiltrating a tissue of a host where the medical device is to be, or has been, implanted with an anti-infective agent, and (b) implanting the medical device into the host; a method for implanting a medical device comprising: (a) infiltrating a tissue of a host where the medical device is to be, or has been, implanted with a polymer; and (b) implanting the medical device into the host; a method for implanting a medical device comprising: (a) infiltrating a tissue of a host where the medical device is to be, or has been, implanted with a composition comprising an anti-fibrotic agent and a polymer, and (b) implanting the medical device into the host; a method for implanting a medical device comprising: (a) infiltrating a tissue of a host where the medical device is to be, or has been, implanted with a composition comprising an anti-infective agent and a polymer, and (b) implanting the medical device into the host; and a method for implanting a medical device comprising: (a) infiltrating a tissue of a host where the medical device is to be, or has been, implanted with a composition comprising an anti-fibrotic agent, an anti-infective agent and a polymer, and (b) implanting the medical device into the host.

For each afore stated aspect, one or more (e.g., any two) of the following features may be used to further define the invention in terms of the device used in the inventive method: the medical device is an esophageal stent; the medical device is a binary stent; the medical device is a colonic stent; and the medical device is a pancreatic stent.

4. Tracheal and Bronchial Stent

In another aspect, the present invention provides a method for implanting a medical device comprising: (a) infiltrating a tissue of a host where the medical device is to be, or has been, implanted with i) an anti-fibrotic agent, ii) an anti-infective agent, iii) a polymer; iv) a composition comprising an anti-fibrotic agent and a polymer, v) a composition comprising an anti-infective agent and a polymer, or vi) a composition comprising an anti-fibrotic agent, an anti-infective agent and a polymer, and (b) implanting the medical device into the host, where the medical device is a tracheal or bronchial stent.

Optionally, in separate aspects, the invention provides: a method for implanting a medical device comprising: (a) infiltrating a tissue of a host where the medical device is to be, or has been, implanted with an anti-fibrotic agent, and (b) implanting the medical device into the host; a method for implanting a medical device comprising: (a) infiltrating a tissue of a host where the medical device is to be, or has been, implanted with an anti-infective agent, and (b) implanting the medical device into the host; a method for implanting a medical device comprising: (a) infiltrating a tissue of a host where the medical device is to be, or has been, implanted with a polymer; and (b) implanting the medical device into the host; a method for implanting a medical device comprising: (a) infiltrating a tissue of a host where the medical device is to be, or has been, implanted with a composition comprising an anti-fibrotic agent and a polymer, and (b) implanting the medical device into the host; a method for implanting a medical device comprising: (a) infiltrating a tissue of a host where the medical device is to be, or has been, implanted with a composition comprising an anti-infective agent and a polymer, and (b) implanting the medical device into the host; and a method for implanting a medical device comprising: (a) infiltrating a tissue of a host where the medical device is to be, or has been, implanted with a composition comprising an anti-fibrotic agent, an anti-infective agent and a polymer, and (b) implanting the medical device into the host.

For each afore stated aspect, one or more (e.g., any two) of the following features may be used to further define the invention in terms of the device used in the inventive method: the medical device is a tracheal stent; the medical device is a bronchial stent; the medical device is a metallic tracheal stent; the medical device is a metallic bronchial stent; the medical device is a polymeric tracheal stent; and the medical device is a polymeric bronchial stent.

5. Genital Urinary Stent

In another aspect, the present invention provides a method for implanting a medical device comprising: (a) infiltrating a tissue of a host where the medical device is to be, or has been, implanted with i) an anti-fibrotic agent, ii) an anti-infective agent, iii) a polymer; iv) a composition comprising an anti-fibrotic agent and a polymer, v) a composition comprising an anti-infective agent and a polymer, or vi) a composition comprising an anti-fibrotic agent, an anti-infective agent and a polymer, and (b) implanting the medical device into the host, where the medical device is a genital urinary stent.

Optionally, in separate aspects, the invention provides: a method for implanting a medical device comprising: (a) infiltrating a tissue of a host where the medical device is to be, or has been, implanted with an anti-fibrotic agent, and (b) implanting the medical device into the host; a method for implanting a medical device comprising: (a) infiltrating a tissue of a host where the medical device is to be, or has been, implanted with an anti-infective agent, and (b) implanting the medical device into the host; a method for implanting a medical device comprising: (a) infiltrating a tissue of a host where the medical device is to be, or has been, implanted with a polymer; and (b) implanting the medical device into the host; a method for implanting a medical device comprising: (a) infiltrating a tissue of a host where the medical device is to be, or has been, implanted with a composition comprising an anti-fibrotic agent and a polymer, and (b) implanting the medical device into the host; a method for implanting a medical device comprising: (a) infiltrating a tissue of a host where the medical device is to be, or has been, implanted with a composition comprising an anti-infective agent and a polymer, and (b) implanting the medical device into the host; and a method for implanting a medical device comprising: (a) infiltrating a tissue of a host where the medical device is to be, or has been, implanted with a composition comprising an anti-fibrotic agent, an anti-infective agent and a polymer, and (b) implanting the medical device into the host.

For each afore stated aspect, one or more (e.g., any two) of the following features may be used to further define the invention in terms of the device used in the inventive method: the medical device is a ureteric stent; the medical device is aurethral stent; the medical device is a fallopian tube stent; the medical device is a prostate stent; the medical device is a metallic genital urinary stent; and the medical device is a polymeric genital urinary stent.

6. Ear and Nose Stent

In another aspect, the present invention provides a method for implanting a medical device comprising: (a) infiltrating a tissue of a host where the medical device is to be, or has been, implanted with i) an anti-fibrotic agent, ii) an anti-infective agent, iii) a polymer; iv) a composition comprising an anti-fibrotic agent and a polymer, v) a composition comprising an anti-infective agent and a polymer, or vi) a composition comprising an anti-fibrotic agent, an anti-infective agent and a polymer, and (b) implanting the medical device into the host, where the medical device is an ear or nose stent.

Optionally, in separate aspects, the invention provides: a method for implanting a medical device comprising: (a) infiltrating a tissue of a host where the medical device is to be, or has been, implanted with an anti-fibrotic agent, and (b) implanting the medical device into the host; a method for implanting a medical device comprising: (a) infiltrating a tissue of a host where the medical device is to be, or has been, implanted with an anti-infective agent, and (b) implanting the medical device into the host; a method for implanting a medical device comprising: (a) infiltrating a tissue of a host where the medical device is to be, or has been, implanted with a polymer; and (b) implanting the medical device into the host; a method for implanting a medical device comprising: (a) infiltrating a tissue of a host where the medical device is to be, or has been, implanted with a composition comprising an anti-fibrotic agent and a polymer, and (b) implanting the medical device into the host; a method for implanting a medical device comprising: (a) infiltrating a tissue of a host where the medical device is to be, or has been, implanted with a composition comprising an anti-infective agent and a polymer, and (b) implanting the medical device into the host; and a method for implanting a medical device comprising: (a) infiltrating a tissue of a host where the medical device is to be, or has been, implanted with a composition comprising an anti-fibrotic agent, an anti-infective agent and a polymer, and (b) implanting the medical device into the host.

For each afore stated aspect, one or more (e.g., any two) of the following features may be used to further define the invention in terms of the device used in the inventive method: the medical device is a lacrimal duct stent; the medical device is an Eustachian tube stent; the medical device is a nasal stent; and the medical device is a sinus stent.

7. Ear Ventilation Tube

In another aspect, the present invention provides a method for implanting a medical device comprising: (a) infiltrating a tissue of a host where the medical device is to be, or has been, implanted with i) an anti-fibrotic agent, ii) an anti-infective agent, iii) a polymer; iv) a composition comprising an anti-fibrotic agent and a polymer, v) a composition comprising an anti-infective agent and a polymer, or vi) a composition comprising an anti-fibrotic agent, an anti-infective agent and a polymer, and (b) implanting the medical device into the host, where the medical device is an ear ventilation tube.

Optionally, in separate aspects, the invention provides: a method for implanting a medical device comprising: (a) infiltrating a tissue of a host where the medical device is to be, or has been, implanted with an anti-fibrotic agent, and (b) implanting the medical device into the host; a method for implanting a medical device comprising: (a) infiltrating a tissue of a host where the medical device is to be, or has been, implanted with an anti-infective agent, and (b) implanting the medical device into the host; a method for implanting a medical device comprising: (a) infiltrating a tissue of a host where the medical device is to be, or has been, implanted with a polymer; and (b) implanting the medical device into the host; a method for implanting a medical device comprising: (a) infiltrating a tissue of a host where the medical device is to be, or has been, implanted with a composition comprising an anti-fibrotic agent and a polymer, and (b) implanting the medical device into the host; a method for implanting a medical device comprising: (a) infiltrating a tissue of a host where the medical device is to be, or has been, implanted with a composition comprising an anti-infective agent and a polymer, and (b) implanting the medical device into the host; and a method for implanting a medical device comprising: (a) infiltrating a tissue of a host where the medical device is to be, or has been, implanted with a composition comprising an anti-fibrotic agent, an anti-infective agent and a polymer, and (b) implanting the medical device into the host.

For each afore stated aspect, one or more (e.g., any two) of the following features may be used to further define the invention in terms of the device used in the inventive method: the medical device is a grommet shaped tube; the medical device is a T-tube; the medical device is a tympanostomy tube; the medical device is a drain tube; the medical device is a tympanic tube; the medical device is an otological tube; the medical device is a myringotomy tube; the medical device is an artifical Eustachian tube; the medical device is an Eustachian tube prosthesis; and the medical device is an Eustachian stent.

8. lntraocular Implant

In another aspect, the present invention provides a method for implanting a medical device comprising: (a) infiltrating a tissue of a host where the medical device is to be, or has been, implanted with i) an anti-fibrotic agent, ii) an anti-infective agent, iii) a polymer; iv) a composition comprising an anti-fibrotic agent and a polymer, v) a composition comprising an anti-infective agent and a polymer, or vi) a composition comprising an anti-fibrotic agent, an anti-infective agent and a polymer, and (b) implanting the medical device into the host, where the medical device is an intraocular implant.

Optionally, in separate aspects, the invention provides: a method for implanting a medical device comprising: (a) infiltrating a tissue of a host where the medical device is to be, or has been, implanted with an anti-fibrotic agent, and (b) implanting the medical device into the host; a method for implanting a medical device comprising: (a) infiltrating a tissue of a host where the medical device is to be, or has been, implanted with an anti-infective agent, and (b) implanting the medical device into the host; a method for implanting a medical device comprising: (a) infiltrating a tissue of a host where the medical device is to be, or has been, implanted with a polymer; and (b) implanting the medical device into the host; a method for implanting a medical device comprising: (a) infiltrating a tissue of a host where the medical device is to be, or has been, implanted with a composition comprising an anti-fibrotic agent and a polymer, and (b) implanting the medical device into the host; a method for implanting a medical device comprising: (a) infiltrating a tissue of a host where the medical device is to be, or has been, implanted with a composition comprising an anti-infective agent and a polymer, and (b) implanting the medical device into the host; and a method for implanting a medical device comprising: (a) infiltrating a tissue of a host where the medical device is to be, or has been, implanted with a composition comprising an anti-fibrotic agent, an anti-infective agent and a polymer, and (b) implanting the medical device into the host.

For each afore stated aspect, one or more (e.g., any two) of the following features may be used to further define the invention in terms of the device used in the inventive method: the medical device is an intraocular lens device for preventing lens opacification; the medical device is a polymethylmethacrylate intraocular lense; the medical device is a silicone intraocular lens; the medical device is an achromatic lens; the medical device is a pseudophako; the medical device is a phakic lens; the medical device is aaphakic lens; the medical device is a multi-focal intraocular lens; the medical device is a hydrophilic and hydrophobic acrylic intraocular lens; the medical device is an intraocular implant; the medical device is an optic lens; the medical device is a rigid gas permeable lens; the medical device is a foldable intraocular lens; the medical device is a rigid intraocular lens; the medical device is a corrective implant for vision impairment; the medical device is an intraocular implant adapted for being used in conjunction with a transplant for the cornea; and the medical device is an intraocular implant adapted for being used in conjunction with treatment of secondary cataract after extracapsular cataract extraction.

9. Medical Device for Treating Hypertropic Scar or Keloid

In another aspect, the present application provide a method for implanting a medical device comprising: (a) infiltrating a tissue of a host where the medical device is to be, or has been, implanted with i) an anti-fibrotic agent, ii) an anti-infective agent, iii) a polymer; iv) a composition comprising an anti-fibrotic agent and a polymer, v) a composition comprising an anti-infective agent and a polymer, or vi) a composition comprising an anti-fibrotic agent, an anti-infective agent and a polymer, and (b) implanting the medical device into the host, where the medical device is a medical device for treating hypertropic scar or keloid.

Optionally, in separate aspects, the invention provides: a method for implanting a medical device comprising: (a) infiltrating a tissue of a host where the medical device is to be, or has been, implanted with an anti-fibrotic agent, and (b) implanting the medical device into the host; a method for implanting a medical device comprising: (a) infiltrating a tissue of a host where the medical device is to be, or has been, implanted with an anti-infective agent, and (b) implanting the medical device into the host; a method for implanting a medical device comprising: (a) infiltrating a tissue of a host where the medical device is to be, or has been, implanted with a polymer; and (b) implanting the medical device into the host; a method for implanting a medical device comprising: (a) infiltrating a tissue of a host where the medical device is to be, or has been, implanted with a composition comprising an anti-fibrotic agent and a polymer, and (b) implanting the medical device into the host; a method for implanting a medical device comprising: (a) infiltrating a tissue of a host where the medical device is to be, or has been, implanted with a composition comprising an anti-infective agent and a polymer, and (b) implanting the medical device into the host; and a method for implanting a medical device comprising: (a) infiltrating a tissue of a host where the medical device is to be, or has been, implanted with a composition comprising an anti-fibrotic agent, an anti-infective agent and a polymer, and (b) implanting the medical device into the host.

For each afore stated aspect, one or more (e.g., any two) of the following features may be used to further define the invention in terms of the device used in the inventive method: the medical device is a device for treating hypertropic scar or keloid that comprises an external tissue expansion device; the medical device is a device for treating hypertropic scar or keloid that comprises a masking element, and wherein the masking element may be pressed onto the scar tissue; and the medical device is a device for treating hypertropic scar or keloid that comprises a locking element and a grasping structure so that the dermal and epidermal layers of a skin wound can be pushed together.

10. Vascular Graft

In another aspect, the present invention provides a method for implanting a medical device comprising: (a) infiltrating a tissue of a host where the medical device is to be, or has been, implanted with i) an anti-fibrotic agent, ii) an anti-infective agent, iii) a polymer; iv) a composition comprising an anti-fibrotic agent and a polymer, v) a composition comprising an anti-infective agent and a polymer, or vi) a composition comprising an anti-fibrotic agent, an anti-infective agent and a polymer, and (b) implanting the medical device into the host, wherein the medical device is a vascular graft.

Optionally, in separate aspects, the invention provides: a method for implanting a medical device comprising: (a) infiltrating a tissue of a host where the medical device is to be, or has been, implanted with an anti-fibrotic agent, and (b) implanting the medical device into the host; a method for implanting a medical device comprising: (a) infiltrating a tissue of a host where the medical device is to be, or has been, implanted with an anti-infective agent, and (b) implanting the medical device into the host; a method for implanting a medical device comprising: (a) infiltrating a tissue of a host where the medical device is to be, or has been, implanted with a polymer; and (b) implanting the medical device into the host; a method for implanting a medical device comprising: (a) infiltrating a tissue of a host where the medical device is to be, or has been, implanted with a composition comprising an anti-fibrotic agent and a polymer, and (b) implanting the medical device into the host; a method for implanting a medical device comprising: (a) infiltrating a tissue of a host where the medical device is to be, or has been, implanted with a composition comprising an anti-infective agent and a polymer, and (b) implanting the medical device into the host; and a method for implanting a medical device comprising: (a) infiltrating a tissue of a host where the medical device is to be, or has been, implanted with a composition comprising an anti-fibrotic agent, an anti-infective agent and a polymer, and (b) implanting the medical device into the host.

For each afore stated aspect, one or more (e.g., any two) of the following features may be used to further define the invention in terms of the device used in the inventive method: the medical device is an extravascular graft; the medical device is an intravascular graft; the medical device is a vascular graft adapted for replacing a blood vessel damaged by aneurysm; the medical device is a vascular graft adapted for replacing a blood vessel damaged by intimal hyperplasia; the medical device is a vascular graft adapted for replacing a blood vessel damaged by thrombosis; the medical device is a vascular graft adapted for providing access to blood vessel; the medical device is a vascular graft adapted for providing an alternative conduit for blood flow through a damaged or diseased area in a vein; the medical device is a vascular graft adapted for providing an alternative conduit for blood flow through a damaged or diseased area in an artery; the medical device is a synthetic bypass graft; the medical device is a femoral-popliteal bypass graft; the medical device is a femoral-femoral bypass graft; the medical device is an axillary-femoral bypass graft; the medical device is a vein graft; the medical device is a peripheral vein graft; the medical device is a coronary vein graft; the medical device is an internal mammary graft; the medical device is a bifurcated vascular graft; the medical device is an intraluminal graft; and the medical device is a prosthetic vascular graft.

11. Hemodialysis Access Device

In another aspect, the present invention provides a method for implanting a medical device comprising: (a) infiltrating a tissue of a host where the medical device is to be, or has been, implanted with i) an anti-fibrotic agent, ii) an anti-infective agent, iii) a polymer; iv) a composition comprising an anti-fibrotic agent and a polymer, v) a composition comprising an anti-infective agent and a polymer, or vi) a composition comprising an anti-fibrotic agent, an anti-infective agent and a polymer, and (b) implanting the medical device into the host, wherein the medical device is a hemodialysis access device.

Optionally, in separate aspects, the invention provides: a method for implanting a medical device comprising: (a) infiltrating a tissue of a host where the medical device is to be, or has been, implanted with an anti-fibrotic agent, and (b) implanting the medical device into the host; a method for implanting a medical device comprising: (a) infiltrating a tissue of a host where the medical device is to be, or has been, implanted with an anti-infective agent, and (b) implanting the medical device into the host; a method for implanting a medical device comprising: (a) infiltrating a tissue of a host where the medical device is to be, or has been, implanted with a polymer; and (b) implanting the medical device into the host; a method for implanting a medical device comprising: (a) infiltrating a tissue of a host where the medical device is to be, or has been, implanted with a composition comprising an anti-fibrotic agent and a polymer, and (b) implanting the medical device into the host; a method for implanting a medical device comprising: (a) infiltrating a tissue of a host where the medical device is to be, or has been, implanted with a composition comprising an anti-infective agent and a polymer, and (b) implanting the medical device into the host; and a method for implanting a medical device comprising: (a) infiltrating a tissue of a host where the medical device is to be, or has been, implanted with a composition comprising an anti-fibrotic agent, an anti-infective agent and a polymer, and (b) implanting the medical device into the host.

For each afore stated aspect, one or more (e.g., any two) of the following features may be used to further define the invention in terms of the device used in the inventive method: the medical device is an AV fistula graft; the medical device is an AV access graft; the medical device is a venous catheter; the medical device is a vascular graft; the medical device is an implantable port; and the medical device is an AV shunt.

12. Medical Device Comprising Film or Mesh

In another aspect, the present invention provides a method for implanting a medical device comprising: (a) infiltrating a tissue of a host where the medical device is to be, or has been, implanted with i) an anti-fibrotic agent, ii) an anti-infective agent, iii) a polymer; iv) a composition comprising an anti-fibrotic agent and a polymer, v) a composition comprising an anti-infective agent and a polymer, or vi) a composition comprising an anti-fibrotic agent, an anti-infective agent and a polymer, and (b) implanting the medical device into the host, wherein the medical device is a device that comprises a film or a mesh.

Optionally, in separate aspects, the invention provides: a method for implanting a medical device comprising: (a) infiltrating a tissue of a host where the medical device is to be, or has been, implanted with an anti-fibrotic agent, and (b) implanting the medical device into the host; a method for implanting a medical device comprising: (a) infiltrating a tissue of a host where the medical device is to be, or has been, implanted with an anti-infective agent, and (b) implanting the medical device into the host; a method for implanting a medical device comprising: (a) infiltrating a tissue of a host where the medical device is to be, or has been, implanted with a polymer; and (b) implanting the medical device into the host; a method for implanting a medical device comprising: (a) infiltrating a tissue of a host where the medical device is to be, or has been, implanted with a composition comprising an anti-fibrotic agent and a polymer, and (b) implanting the medical device into the host; a method for implanting a medical device comprising: (a) infiltrating a tissue of a host where the medical device is to be, or has been, implanted with a composition comprising an anti-infective agent and a polymer, and (b) implanting the medical device into the host; and a method for implanting a medical device comprising: (a) infiltrating a tissue of a host where the medical device is to be, or has been, implanted with a composition comprising an anti-fibrotic agent, an anti-infective agent and a polymer, and (b) implanting the medical device into the host.

For each afore stated aspect, one or more (e.g., any two) of the following features may be used to further define the invention in terms of the device used in the inventive method: the medical device is a surgical barrier; the medical device is a surgical adhesion barrier; the medical device is a surgical sheet; the medical device is a surgical patch; the medical device is a surgical wrap; the medical device is a vascular wrap; the medical device is a perivascular wrap; the medical device is an adventitial wrap; the medical device is a periadventitital wrap; the medical device is an adventitial sheet; the medical device is a perivascular mesh; the medical device is a bandage; the medical device is a liquid bandage; the medical device is a surgical dressing; the medical device is a gauze; the medical device is a fabric; the medical device is a tape; the medical device is a surgical membrane; the medical device is a polymer matrix; the medical device is a tissue covering; the medical device is a surgical matrix; the medical device is an envelope; and the medical device is a tissue covering.

13. Glaucoma Drainage Device

In another aspect, the present invention provides a method for implanting a medical device comprising: (a) infiltrating a tissue of a host where the medical device is to be, or has been, implanted with i) an anti-fibrotic agent, ii) an anti-infective agent, iii) a polymer; iv) a composition comprising an anti-fibrotic agent and a polymer, v) a composition comprising an anti-infective agent and a polymer, or vi) a composition comprising an anti-fibrotic agent, an anti-infective agent and a polymer, and (b) implanting the medical device into the host, wherein the medical device is a glaucoma drainage device.

Optionally, in separate aspects, the invention provides: a method for implanting a medical device comprising: (a) infiltrating a tissue of a host where the medical device is to be, or has been, implanted with an anti-fibrotic agent, and (b) implanting the medical device into the host; a method for implanting a medical device comprising: (a) infiltrating a tissue of a host where the medical device is to be, or has been, implanted with an anti-infective agent, and (b) implanting the medical device into the host; a method for implanting a medical device comprising: (a) infiltrating a tissue of a host where the medical device is to be, or has been, implanted with a polymer; and (b) implanting the medical device into the host; a method for implanting a medical device comprising: (a) infiltrating a tissue of a host where the medical device is to be, or has been, implanted with a composition comprising an anti-fibrotic agent and a polymer, and (b) implanting the medical device into the host; a method for implanting a medical device comprising: (a) infiltrating a tissue of a host where the medical device is to be, or has been, implanted with a composition comprising an anti-infective agent and a polymer, and (b) implanting the medical device into the host; and a method for implanting a medical device comprising: (a) infiltrating a tissue of a host where the medical device is to be, or has been, implanted with a composition comprising an anti-fibrotic agent, an anti-infective agent and a polymer, and (b) implanting the medical device into the host.

In certain embodiments, the medical device is a glaucoma drainage device comprising a plate and a tube.

14. Prosthetic Heart Valve or Component Thereof

In another aspect, the present invention provides a method for implanting a medical device comprising: (a) infiltrating a tissue of a host where the medical device is to be, or has been, implanted with i) an anti-fibrotic agent, ii) an anti-infective agent, iii) a polymer; iv) a composition comprising an anti-fibrotic agent and a polymer, v) a composition comprising an anti-infective agent and a polymer, or vi) a composition comprising an anti-fibrotic agent, an anti-infective agent and a polymer, and (b) implanting the medical device into the host, wherein the medical device is a prosthetic heart valve or a component thereof.

Optionally, in separate aspects, the invention provides: a method for implanting a medical device comprising: (a) infiltrating a tissue of a host where the medical device is to be, or has been, implanted with an anti-fibrotic agent, and (b) implanting the medical device into the host; a method for implanting a medical device comprising: (a) infiltrating a tissue of a host where the medical device is to be, or has been, implanted with an anti-infective agent, and (b) implanting the medical device into the host; a method for implanting a medical device comprising: (a) infiltrating a tissue of a host where the medical device is to be, or has been, implanted with a polymer; and (b) implanting the medical device into the host; a method for implanting a medical device comprising: (a) infiltrating a tissue of a host where the medical device is to be, or has been, implanted with a composition comprising an anti-fibrotic agent and a polymer, and (b) implanting the medical device into the host; a method for implanting a medical device comprising: (a) infiltrating a tissue of a host where the medical device is to be, or has been, implanted with a composition comprising an anti-infective agent and a polymer, and (b) implanting the medical device into the host; and a method for implanting a medical device comprising: (a) infiltrating a tissue of a host where the medical device is to be, or has been, implanted with a composition comprising an anti-fibrotic agent, an anti-infective agent and a polymer, and (b) implanting the medical device into the host.

For each afore stated aspect, one or more (e.g., any two) of the following features may be used to further define the invention in terms of the device used in the inventive method: the medical device is a mechanical prosthetic heart valve; the medical device is a bioprosthetic heart valve; the medical device is an implantable annular ring for receiving a prosthetic heart valve; the medical device is a suture ring having an outer peripheral tapered thread for attaching a heart valve prosthesis; and the medical device is a suture ring for a mechanical heart valve.

15. Penile Implant

In another aspect, the present invention provides a method for implanting a medical device comprising: (a) infiltrating a tissue of a host where the medical device is to be, or has been, implanted with i) an anti-fibrotic agent, ii) an anti-infective agent, iii) a polymer; iv) a composition comprising an anti-fibrotic agent and a polymer, v) a composition comprising an anti-infective agent and a polymer, or vi) a composition comprising an anti-fibrotic agent, an anti-infective agent and a polymer, and (b) implanting the medical device into the host, wherein the medical device is a penile implant.

Optionally, in separate aspects, the invention provides: a method for implanting a medical device comprising: (a) infiltrating a tissue of a host where the medical device is to be, or has been, implanted with an anti-fibrotic agent, and (b) implanting the medical device into the host; a method for implanting a medical device comprising: (a) infiltrating a tissue of a host where the medical device is to be, or has been, implanted with an anti-infective agent, and (b) implanting the medical device into the host; a method for implanting a medical device comprising: (a) infiltrating a tissue of a host where the medical device is to be, or has been, implanted with a polymer; and (b) implanting the medical device into the host; a method for implanting a medical device comprising: (a) infiltrating a tissue of a host where the medical device is to be, or has been, implanted with a composition comprising an anti-fibrotic agent and a polymer, and (b) implanting the medical device into the host; a method for implanting a medical device comprising: (a) infiltrating a tissue of a host where the medical device is to be, or has been, implanted with a composition comprising an anti-infective agent and a polymer, and (b) implanting the medical device into the host; and a method for implanting a medical device comprising: (a) infiltrating a tissue of a host where the medical device is to be, or has been, implanted with a composition comprising an anti-fibrotic agent, an anti-infective agent and a polymer, and (b) implanting the medical device into the host.

For each afore stated aspect, one or more (e.g., any two) of the following features may be used to further define the invention in terms of the device used in the inventive method: the medical device is a penile implant that is a flexible rod; the medical device is a penile implant that is a hinged rod; and the medical device is a penile implant that is an inflatable device with a pump.

16. Endotracheal or Tracheostomy Tube

In another aspect, the present invention provides a method for implanting a medical device comprising: (a) infiltrating a tissue of a host where the medical device is to be, or has been, implanted with i) an anti-fibrotic agent, ii) an anti-infective agent, iii) a polymer; iv) a composition comprising an anti-fibrotic agent and a polymer, v) a composition comprising an anti-infective agent and a polymer, or vi) a composition comprising an anti-fibrotic agent, an anti-infective agent and a polymer, and (b) implanting the medical device into the host, wherein the medical device is an endotracheal or tracheostomy tube.

Optionally, in separate aspects, the invention provides: a method for implanting a medical device comprising: (a) infiltrating a tissue of a host where the medical device is to be, or has been, implanted with an anti-fibrotic agent, and (b) implanting the medical device into the host; a method for implanting a medical device comprising: (a) infiltrating a tissue of a host where the medical device is to be, or has been, implanted with an anti-infective agent, and (b) implanting the medical device into the host; a method for implanting a medical device comprising: (a) infiltrating a tissue of a host where the medical device is to be, or has been, implanted with a polymer; and (b) implanting the medical device into the host; a method for implanting a medical device comprising: (a) infiltrating a tissue of a host where the medical device is to be, or has been, implanted with a composition comprising an anti-fibrotic agent and a polymer, and (b) implanting the medical device into the host; a method for implanting a medical device comprising: (a) infiltrating a tissue of a host where the medical device is to be, or has been, implanted with a composition comprising an anti-infective agent and a polymer, and (b) implanting the medical device into the host; and a method for implanting a medical device comprising: (a) infiltrating a tissue of a host where the medical device is to be, or has been, implanted with a composition comprising an anti-fibrotic agent, an anti-infective agent and a polymer, and (b) implanting the medical device into the host.

For each afore stated aspect, one or more (e.g., any two) of the following features may be used to further define the invention in terms of the device used in the inventive method: the medical device is an endotracheal tube; the medical device is an endotracheal tube with a single lumen; the medical device is an endotracheal tube with double lumens; and the medical device is a tracheostomy tube.

17. Peritoneal Dialysis Catheter In another aspect, the present invention provides a method for implanting a medical device comprising: (a) infiltrating a tissue of a host where the medical device is to be, or has been, implanted with i) an anti-fibrotic agent, ii) an anti-infective agent, iii) a polymer; iv) a composition comprising an anti-fibrotic agent and a polymer, v) a composition comprising an anti-infective agent and a polymer, or vi) a composition comprising an anti-fibrotic agent, an anti-infective agent and a polymer, and (b) implanting the medical device into the host, wherein the medical device is a peritoneal dialysis catheter.

Optionally, in separate aspects, the invention provides: a method for implanting a medical device comprising: (a) infiltrating a tissue of a host where the medical device is to be, or has been, implanted with an anti-fibrotic agent, and (b) implanting the medical device into the host; a method for implanting a medical device comprising: (a) infiltrating a tissue of a host where the medical device is to be, or has been, implanted with an anti-infective agent, and (b) implanting the medical device into the host; a method for implanting a medical device comprising: (a) infiltrating a tissue of a host where the medical device is to be, or has been, implanted with a polymer; and (b) implanting the medical device into the host; a method for implanting a medical device comprising: (a) infiltrating a tissue of a host where the medical device is to be, or has been, implanted with a composition comprising an anti-fibrotic agent and a polymer, and (b) implanting the medical device into the host; a method for implanting a medical device comprising: (a) infiltrating a tissue of a host where the medical device is to be, or has been, implanted with a composition comprising an anti-infective agent and a polymer, and (b) implanting the medical device into the host; and a method for implanting a medical device comprising: (a) infiltrating a tissue of a host where the medical device is to be, or has been, implanted with a composition comprising an anti-fibrotic agent, an anti-infective agent and a polymer, and (b) implanting the medical device into the host.

In certain embodiments, the medical device is a peritoneal dialysis catheter is adapted for delivering a drug to the peritoneum.

18. Central Nervous System Shunt or Pressure Monitoring Device

In another aspect, the present invention provides a method for implanting a medical device comprising: (a) infiltrating a tissue of a host where the medical device is to be, or has been, implanted with i) an anti-fibrotic agent, ii) an anti-infective agent, iii) a polymer; iv) a composition comprising an anti-fibrotic agent and a polymer, v) a composition comprising an anti-infective agent and a polymer, or vi) a composition comprising an anti-fibrotic agent, an anti-infective agent and a polymer, and (b) implanting the medical device into the host, wherein the medical device is a central nervous system shunt or a pressure monitoring.

Optionally, in separate aspects, the invention provides: a method for implanting a medical device comprising: (a) infiltrating a tissue of a host where the medical device is to be, or has been, implanted with an anti-fibrotic agent, and (b) implanting the medical device into the host; a method for implanting a medical device comprising: (a) infiltrating a tissue of a host where the medical device is to be, or has been, implanted with an anti-infective agent, and (b) implanting the medical device into the host; a method for implanting a medical device comprising: (a) infiltrating a tissue of a host where the medical device is to be, or has been, implanted with a polymer; and (b) implanting the medical device into the host; a method for implanting a medical device comprising: (a) infiltrating a tissue of a host where the medical device is to be, or has been, implanted with a composition comprising an anti-fibrotic agent and a polymer, and (b) implanting the medical device into the host; a method for implanting a medical device comprising: (a) infiltrating a tissue of a host where the medical device is to be, or has been, implanted with a composition comprising an anti-infective agent and a polymer, and (b) implanting the medical device into the host; and a method for implanting a medical device comprising: (a) infiltrating a tissue of a host where the medical device is to be, or has been, implanted with a composition comprising an anti-fibrotic agent, an anti-infective agent and a polymer, and (b) implanting the medical device into the host.

For each afore stated aspect, one or more (e.g., any two) of the following features may be used to further define the invention in terms of the device used in the inventive method: the medical device is a ventriculopleural shunt; the medical device is a jugular vein shunt; the medical device is a vena cava shunt; the medical device is a ventriculoperitoneal shunt; the medical device is a gallbladder shunt; the medical device is a peritoneum shunt; the medical device is an external ventricular drainage device; the medical device is an intracranial pressure monitoring device; the medical device is a dural patch; the medical device is an implant to prevent epidural fibrosis post-laminectomy; the medical device is a device for continuous subarachnoid infusion; the medical device is a drainage shunt useful for draining fluids in the brain; and the medical device is a pressure monitoring device.

19. Inferior Vena Cava Filter

In certain embodiments, the present invention provides a method for implanting a medical device comprising: (a) infiltrating a tissue of a host where the medical device is to be, or has been, implanted with i) an anti-fibrotic agent, ii) an anti-infective agent, iii) a polymer; iv) a composition comprising an anti-fibrotic agent and a polymer, v) a composition comprising an anti-infective agent and a polymer, or vi) a composition comprising an anti-fibrotic agent, an anti-infective agent and a polymer, and (b) implanting the medical device into the host, wherein the medical device is an inferior vena cava filter.

Optionally, in separate aspects, the invention provides: a method for implanting a medical device comprising: (a) infiltrating a tissue of a host where the medical device is to be, or has been, implanted with an anti-fibrotic agent, and (b) implanting the medical device into the host; a method for implanting a medical device comprising: (a) infiltrating a tissue of a host where the medical device is to be, or has been, implanted with an anti-infective agent, and (b) implanting the medical device into the host; a method for implanting a medical device comprising: (a) infiltrating a tissue of a host where the medical device is to be, or has been, implanted with a polymer; and (b) implanting the medical device into the host; a method for implanting a medical device comprising: (a) infiltrating a tissue of a host where the medical device is to be, or has been, implanted with a composition comprising an anti-fibrotic agent and a polymer, and (b) implanting the medical device into the host; a method for implanting a medical device comprising: (a) infiltrating a tissue of a host where the medical device is to be, or has been, implanted with a composition comprising an anti-infective agent and a polymer, and (b) implanting the medical device into the host; and a method for implanting a medical device comprising: (a) infiltrating a tissue of a host where the medical device is to be, or has been, implanted with a composition comprising an anti-fibrotic agent, an anti-infective agent and a polymer, and (b) implanting the medical device into the host.

For each afore stated aspect, one or more (e.g., any two) of the following features may be used to further define the invention in terms of the device used in the inventive method: the medical device is a vascular filter; the medical device is a blood filter; the medical device is a caval filter; the medical device is a vena cava filter; the medical device is a thrombus filter; the medical device is an antimigration filter; the medical device is a percutaneous filter system; the medical device is an intravascular trap; the medical device is an intravascular filter; the medical device is a clot filter; the medical device is a vein filter; and the medical device is a body vessel filter.

20. Gastrointestinal Device

In another aspect, the present invention provides a method for implanting a medical device comprising: (a) infiltrating a tissue of a host where the medical device is to be, or has been, implanted with i) an anti-fibrotic agent, ii) an anti-infective agent, iii) a polymer; iv) a composition comprising an anti-fibrotic agent and a polymer, v) a composition comprising an anti-infective agent and a polymer, or vi) a composition comprising an anti-fibrotic agent, an anti-infective agent and a polymer, and (b) implanting the medical device into the host, wherein the medical device is gastrointestinal device.

Optionally, in separate aspects, the-invention provides: a method for implanting a medical device comprising: (a) infiltrating a tissue of a host where the medical device is to be, or has been, implanted with an anti-fibrotic agent, and (b) implanting the medical device into the host; a method for implanting a medical device comprising: (a) infiltrating a tissue of a host where the medical device is to be, or has been, implanted with an anti-infective agent, and (b) implanting the medical device into the host; a method for implanting a medical device comprising: (a) infiltrating a tissue of a host where the medical device is to be, or has been, implanted with a polymer; and (b) implanting the medical device into the host; a method for implanting a medical device comprising: (a) infiltrating a tissue of a host where the medical device is to be, or has been, implanted with a composition comprising an anti-fibrotic agent and a polymer, and (b) implanting the medical device into the host; a method for implanting a medical device comprising: (a) infiltrating a tissue of a host where the medical device is to be, or has been, implanted with a composition comprising an anti-infective agent and a polymer, and (b) implanting the medical device into the host; and a method for implanting a medical device comprising: (a) infiltrating a tissue of a host where the medical device is to be, or has been, implanted with a composition comprising an anti-fibrotic agent, an anti-infective agent and a polymer, and (b) implanting the medical device into the host.

For each afore stated aspect, one or more (e.g., any two) of the following features may be used to further define the invention in terms of the device used in the inventive method: the medical device is a drainage tube; the medical device is a feeding tube; the medical device is a portosystemic shunt; the medical device is a shunt for ascite; the medical device is a nasogastric or nasoenteral tube; the medical device is a gastrostomy or percutaneous feeding tube; the medical device is a jejunostomy endoscopic tube; the medical device is a colostomy device; the medical device is a binary T-tube; the medical device is biopsy forceps; the medical device is a biliary stone removal device; the medical device is an endoscopic retrograde cholangiopancretography device; the medical device is a dilation balloon; the medical device is an enteral feeding device; the medical device is a stent; the medical device is a low profile device; the medical device is a virtual colonoscopy device; the medical device is a capsule endoscope; the medical device is a retrieval device; the medical device is a gastrointestinal device adapted for examining the interior of the gastrointestinal tract; the medical device is a gastrointestinal device adapted for irrigation or aspiration of the gastrointestinal tract; the medical device is a colostomy device; the medical device is a mechanical hemostatic device adapted for control gastrointestinal bleeding; the medical device is a gastrointestinal device adapted for cleaning blocked the gastrointestinal tract; the medical device is a gastrointestinal device for providing communication between two bodily systems; the medical device is a poortosystemic shunt; and the medical device is a dilatation catheter.

21. Central Venous Catheter In another aspect, the present invention provides a method for implanting a medical device comprising: (a) infiltrating a tissue of a host where the medical device is to be, or has been, implanted with i) an anti-fibrotic agent, ii) an anti-infective agent, iii) a polymer; iv) a composition comprising an anti-fibrotic agent and a polymer, v) a composition comprising an anti-infective agent and a polymer, or vi) a composition comprising an anti-fibrotic agent, an anti-infective agent and a polymer, and (b) implanting the medical device into the host, wherein the medical device is a central venous catheter.

Optionally, in separate aspects, the invention provides: a method for implanting a medical device comprising: (a) infiltrating a tissue of a host where the medical device is to be, or has been, implanted with an anti-fibrotic agent, and (b) implanting the medical device into the host; a method for implanting a medical device comprising: (a) infiltrating a tissue of a host where the medical device is to be, or has been, implanted with an anti-infective agent, and (b) implanting the medical device into the host; a method for implanting a medical device comprising: (a) infiltrating a tissue of a host where the medical device is to be, or has been, implanted with a polymer; and (b) implanting the medical device into the host; a method for implanting a medical device comprising: (a) infiltrating a tissue of a host where the medical device is to be, or has been, implanted with a composition comprising an anti-fibrotic agent and a polymer, and (b) implanting the medical device into the host; a method for implanting a medical device comprising: (a) infiltrating a tissue of a host where the medical device is to be, or has been, implanted with a composition comprising an anti-infective agent and a polymer, and (b) implanting the medical device into the host; and a method for implanting a medical device comprising: (a) infiltrating a tissue of a host where the medical device is to be, or has been, implanted with a composition comprising an anti-fibrotic agent, an anti-infective agent and a polymer, and (b) implanting the medical device into the host.

For each afore stated aspect, one or more (e.g., any two) of the following features may be used to further define the invention in terms of the device used in the inventive method: the medical device is a central venous catheter with a cuff; the medical device is a central venous catheter without a cuff; the medical device is a central venous catheter with a flange; the medical device is a central venous catheter without a flange; the medical device is a central venous catheter adapted for providing access to the circulatory system; the medical device is a central venous catheter adapted for providing multiple conduits for accessing the circulatory system; the medical device is a central venous catheter comprises a mean for preventing infection as a result of long term use; the medical device is a central venous catheter adaptable for being used with an apparatus that provides a means of controlling the injection or withdrawal of bodily fluids through the central venous catheter; the medical device is a parenteral nutrition catheter; the medical device is a peripherally inserted central venous catheter; the medical device is a flow directed balloon tipped pulmonary artery catheter; and the medical device is a long term central venous access catheter.

22. Ventricular Assist Device

In another aspect, the present invention provides a method for implanting a medical device comprising: (a) infiltrating a tissue of a host where the medical device is to be, or has been, implanted with i) an anti-fibrotic agent, ii) an anti-infective agent, iii) a polymer; iv) a composition comprising an anti-fibrotic agent and a polymer, v) a composition comprising an anti-infective agent and a polymer, or vi) a composition comprising an anti-fibrotic agent, an anti-infective agent and a polymer, and (b) implanting the medical device into the host, wherein the medical device is a ventricular assist device.

Optionally, in separate aspects, the invention provides: a method for implanting a medical device comprising: (a) infiltrating a tissue of a host where the medical device is to be, or has been, implanted with an anti-fibrotic agent, and (b) implanting the medical device into the host; a method for implanting a medical device comprising: (a) infiltrating a tissue of a host where the medical device is to be, or has been, implanted with an anti-infective agent, and (b) implanting the medical device into the host; a method for implanting a medical device comprising: (a) infiltrating a tissue of a host where the medical device is to be, or has been, implanted with a polymer; and (b) implanting the medical device into the host; a method for implanting a medical device comprising: (a) infiltrating a tissue of a host where the medical device is to be, or has been, implanted with a composition comprising an anti-fibrotic agent and a polymer, and (b) implanting the medical device into the host; a method for implanting a medical device comprising: (a) infiltrating a tissue of a host where the medical device is to be, or has been, implanted with a composition comprising an anti-infective agent and a polymer, and (b) implanting the medical device into the host; and a method for implanting a medical device comprising: (a) infiltrating a tissue of a host where the medical device is to be, or has been, implanted with a composition comprising an anti-fibrotic agent, an anti-infective agent and a polymer, and (b) implanting the medical device into the host.

For each afore stated aspect, one or more (e.g., any two) of the following features may be used to further define the invention in terms of the device used in the inventive method: the medical device is a left ventricular assist device; the medical device is a right ventricular assist device; the medical device is a biventricular assist device; the medical device is a cardiac assist device; the medical device is a mechanical assist device; the medical device is an artificial cardiac assist device; the medical device is an implantable heart assist system; the medical device is a heart assist pump; and the medical device is an intra-ventricular cardiac assist device.

23. Spinal Implant

In another aspect, the present invention provides a method for implanting a medical device comprising: (a) infiltrating a tissue of a host where the medical device is to be, or has been, implanted with i) an anti-fibrotic agent, ii) an anti-infective agent, iii) a polymer; iv) a composition comprising an anti-fibrotic agent and a polymer, v) a composition comprising an anti-infective agent and a polymer, or vi) a composition comprising an anti-fibrotic agent, an anti-infective agent and a polymer, and (b) implanting the medical device into the host, wherein the medical device is a spinal implant.

Optionally, in separate aspects, the invention provides: a method for implanting a medical device comprising: (a) infiltrating a tissue of a host where the medical device is to be, or has been, implanted with an anti-fibrotic agent, and (b) implanting the medical device into the host; a method for implanting a medical device comprising: (a) infiltrating a tissue of a host where the medical device is to be, or has been, implanted with an anti-infective agent, and (b) implanting the medical device into the host; a method for implanting a medical device comprising: (a) infiltrating a tissue of a host where the medical device is to be, or has been, implanted with a polymer; and (b) implanting the medical device into the host; a method for implanting a medical device comprising: (a) infiltrating a tissue of a host where the medical device is to be, or has been, implanted with a composition comprising an anti-fibrotic agent and a polymer, and (b) implanting the medical device into the host; a method for implanting a medical device comprising: (a) infiltrating a tissue of a host where the medical device is to be, or has been, implanted with a composition comprising an anti-infective agent and a polymer, and (b) implanting the medical device into the host; and a method for implanting a medical device comprising: (a) infiltrating a tissue of a host where the medical device is to be, or has been, implanted with a composition comprising an anti-fibrotic agent, an anti-infective agent and a polymer, and (b) implanting the medical device into the host.

For each afore stated aspect, one or more (e.g., any two) of the following features may be used to further define the invention in terms of the device used in the inventive method: the medical device is a spinal disc; the medical device is a vertebral implant; the medical device is a vertebral disc prosthesis; the medical device is a lumbar disc implant; the medical device is a cervical disc implant; the medical device is a intervertebral disc; the medical device is a spinal prosthesis; the medical device is a artificial disc; the medical device is a spinal disc endoprosthesis; the medical device is an intervertebral implant; the medical device is an implantable spinal graft; the medical device is an implantable bone graft; the medical device is an artificial lumbar discs; the medical device is a spinal nucleus implant; the medical device is an intervertebral disc spacer; the medical device is a fusion cage; the medical device is a fusion basket; the medical device is a fusion cage apparatus; the medical device is an interbody cage; the medical device is an interbody implant; the medical device is a fusion cage anchoring device; the medical device is a bone fixation apparatus; the medical device is a fusion stabilization chamber; the medical device is an anchoring bone plate; and the medical device is a bone screw.

24. Electrical Device

In another aspect, the present invention provides a method for implanting a medical device comprising: (a) infiltrating a tissue of a host where the medical device is to be, or has been, implanted with i) an anti-fibrotic agent, ii) an anti-infective agent, iii) a polymer; iv) a composition comprising an anti-fibrotic agent and a polymer, v) a composition comprising an anti-infective agent and a polymer, or vi) a composition comprising an anti-fibrotic agent, an anti-infective agent and a polymer, and (b) implanting the medical device into the host, wherein the medical device is an electrical device.

Optionally, in separate aspects, the invention provides: a method for implanting a medical device comprising: (a) infiltrating a tissue of a host where the medical device is to be, or has been, implanted with an anti-fibrotic agent, and (b) implanting the medical device into the host; a method for implanting a medical device comprising: (a) infiltrating a tissue of a host where the medical device is to be, or has been, implanted with an anti-infective agent, and (b) implanting the medical device into the host; a method for implanting a medical device comprising: (a) infiltrating a tissue of a host where the medical device is to be, or has been, implanted with a polymer; and (b) implanting the medical device into the host; a method for implanting a medical device comprising: (a) infiltrating a tissue of a host where the medical device is to be, or has been, implanted with a composition comprising an anti-fibrotic agent and a polymer, and (b) implanting the medical device into the host; a method for implanting a medical device comprising: (a) infiltrating a tissue of a host where the medical device is to be, or has been, implanted with a composition comprising an anti-infective agent and a polymer, and (b) implanting the medical device into the host; and a method for implanting a medical device comprising: (a) infiltrating a tissue of a host where the medical device is to be, or has been, implanted with a composition comprising an anti-fibrotic agent, an anti-infective agent and a polymer, and (b) implanting the medical device into the host.

For each afore stated aspect, one or more (e.g., any two) of the following features may be used to further define the invention in terms of the device used in the inventive method: the medical device is a neurostimulator; the medical device is a spinal cord stimulator; the medical device is a brain stimulator; the medical device is a vagus nerve stimulator; the medical device is a sacral nerve stimulator; the medical device is a gastric nerve stimulator; the medical device is an auditory nerve stimulator; the medical device delivers stimulation to organs; the medical device delivers stimulation to bone; the medical device delivers stimulation to muscles; the medical device delivers stimulation to tissues; the medical device is a device for continuous subarachnoid infusion; the medical device is an implantable electrode; the medical device is an implantable pulse generator; the medical device is an electrical lead; the medical device is a stimulation lead; the medical device is a simulation catheter lead; the medical device is cochlear implant; the medical device is a microstimulator; the medical device is battery powered; the medical device is radio frequency powered; the medical device is both battery and radio frequency powered; the medical device is a cardiac rhythm management device; the medical device is a cardiac pacemaker; the medical device is an implantable cardioverter defibrillator system; the medical device is a cardiac lead; the medical device is a pacer lead; the medical device is an endocardial lead; the medical device is a cardioversion/defibrillator lead; the medical device is an epicardial lead; the medical device is an epicardial defibrillator lead; the medical device is a patch defibrillator; the medical device is a patch defibrillator lead; the medical device is an electrical patch; the medical device is a transvenous lead; the medical device is an active fixation lead; the medical device is a passive fixation lead; the medical device is a sensing lead; the medical device is a defibrillator; the medical device is an implantable sensor; the medical device is a left ventricular assist device; the medical device is a pulse generator; the medical device is a patch lead; the medical device is an electrical patch; the medical device is a cardiac stimulator; the medical device is an electrical deviceable sensor; the medical device is an electrical deviceable pump; the medical device is a dural patch; the medical device is a ventricular peritoneal shunt; the medical device is a ventricular atrial shunt; the medical device is an electrical device adapted for treating or preventing epidural fibrosis post-laminectomy; the medical device is an electrical device adapted for treating or preventing cardiac rhythm abnormalities; the medical device is an electrical device adapted for treating or preventing atrial rhythm abnormalities; the medical device is an electrical device adapted for treating or preventing conduction abnormalities; the medical device is an electrical device adapted for treating or preventing ventricular rhythm abnormalities; the medical device is an electrical device adapted for treating or preventing pain; the medical device is an electrical device adapted for treating or preventing epilepsy; the medical device is an electrical device adapted for treating or preventing Parkinson's disease; the medical device is an electrical device adapted for treating or preventing movement disorders; the medical device is an electrical device adapted for treating or preventing obesity; the medical device is an electrical device adapted for treating or preventing depression; the medical device is an electrical device adapted for treating or preventing anxiety; the medical device is an electrical device adapted for treating or preventing hearing loss; the medical device is an electrical device adapted for treating or preventing ulcers; the medical device is an electrical device adapted for treating or preventing deep vein thrombosis; the medical device is an electrical device adapted for treating or preventing muscular atrophy; the medical device is an electrical device adapted for treating or preventing joint stiffness; the medical device is an electrical device adapted for treating or preventing muscle spasms; the medical device is an electrical device adapted for treating or preventing osteoporosis; the medical device is an electrical device adapted for treating or preventing scoliosis; the medical device is an electrical device adapted for treating or preventing spinal disc degeneration; the medical device is an electrical device adapted for treating or preventing spinal cord injury; the medical device is an electrical device adapted for treating or preventing urinary dysfunction; the medical device is an electrical device adapted for treating or preventing gastroparesis; the medical device is an electrical device adapted for treating or preventing malignancy; the medical device is an electrical device adapted for treating or preventing arachnoiditis; the medical device is an electrical device adapted for treating or preventing chronic disease; the medical device is an electrical device adapted for treating or preventing migraine; the medical device is an electrical device adapted for treating or preventing sleep disorders; the medical device is an electrical device adapted for treating or preventing dementia; and the medical device is an electrical device adapted for treating or preventing Alzheimer's disease.

25. Sensor

In another aspect, the present invention provides a method for implanting a medical device comprising: (a) infiltrating a tissue of a host where the medical device is to be, or has been, implanted with i) an anti-fibrotic agent, ii) an anti-infective agent, iii) a polymer; iv) a composition comprising an anti-fibrotic agent and a polymer, v) a composition comprising an anti-infective agent and a polymer, or vi) a composition comprising an anti-fibrotic agent, an anti-infective agent and a polymer, and (b) implanting the medical device into the host, wherein the medical device is a sensor.

Optionally, in separate aspects, the invention provides: a method for implanting a medical device comprising: (a) infiltrating a tissue of a host where the medical device is to be, or has been, implanted with an anti-fibrotic agent, and (b) implanting the medical device into the host; a method for implanting a medical device comprising: (a) infiltrating a tissue of a host where the medical device is to be, or has been, implanted with an anti-infective agent, and (b) implanting the medical device into the host; a method for implanting a medical device comprising: (a) infiltrating a tissue of a host where the medical device is to be, or has been, implanted with a polymer; and (b) implanting the medical device into the host; a method for implanting a medical device comprising: (a) infiltrating a tissue of a host where the medical device is to be, or has been, implanted with a composition comprising an anti-fibrotic agent and a polymer, and (b) implanting the medical device into the host; a method for implanting a medical device comprising: (a) infiltrating a tissue of a host where the medical device is to be, or has been, implanted with a composition comprising an anti-infective agent and a polymer, and (b) implanting the medical device into the host; and a method for implanting a medical device comprising: (a) infiltrating a tissue of a host where the medical device is to be, or has been, implanted with a composition comprising an anti-fibrotic agent, an anti-infective agent and a polymer, and (b) implanting the medical device into the host.

For each afore stated aspect, one or more (e.g., any two) of the following features may be used to further define the invention in terms of the device used in the inventive method: the medical device is a blood or tissue glucose monitor; the medical device is an electrolyte sensor; the medical device is a blood constituent sensor; the medical device is a temperature sensor; the medical device is a pH sensor; the medical device is an optical sensor; the medical device is an amperometric sensor; the medical device is a pressure sensor; the medical device is a biosensor; the medical device is a sensing transponder; the medical device is a strain sensor; the medical device is a magnetoresistive sensor; the medical device is a cardiac sensor; the medical device is a respiratory sensor; the medical device is an auditory sensor; the medical device is a metabolite sensor; the medical device is a sensor that detects mechanical changes; the medical device is a sensor that detects physical changes; the medical device is a sensor that detects electrochemical changes; the medical device is a sensor that detects magnetic changes; the medical device is a sensor that detects acceleration changes; the medical device is a sensor that detects ionizing radiation changes; the medical device is a sensor that detects acoustic wave changes; the medical device is a sensor that detects chemical changes; the medical device is a sensor that detects drug concentration changes; the medical device is a sensor that detects hormone changes; and the medical device is a sensor that detects barometric changes.

26. Pump

In another aspect, the present invention provides a method for implanting a medical device comprising: (a) infiltrating a tissue of a host where the medical device is to be, or has been, implanted with i) an anti-fibrotic agent, ii) an anti-infective agent, iii) a polymer; iv) a composition comprising an anti-fibrotic agent and a polymer, v) a composition comprising an anti-infective agent and a polymer, or vi) a composition comprising an anti-fibrotic agent, an anti-infective agent and a polymer, and (b) implanting the medical device into the host, wherein the medical device is a pump.

Optionally, in separate aspects, the invention provides: a method for implanting a medical device comprising: (a) infiltrating a tissue of a host where the medical device is to be, or has been, implanted with an anti-fibrotic agent, and (b) implanting the medical device into the host; a method for implanting a medical device comprising: (a) infiltrating a tissue of a host where the medical device is to be, or has been, implanted with an anti-infective agent, and (b) implanting the medical device into the host; a method for implanting a medical device comprising: (a) infiltrating a tissue of a host where the medical device is to be, or has been, implanted with a polymer; and (b) implanting the medical device into the host; a method for implanting a medical device comprising: (a) infiltrating a tissue of a host where the medical device is to be, or has been, implanted with a composition comprising an anti-fibrotic agent and a polymer, and (b) implanting the medical device into the host; a method for implanting a medical device comprising: (a) infiltrating a tissue of a host where the medical device is to be, or has been, implanted with a composition comprising an anti-infective agent and a polymer, and (b) implanting the medical device into the host; and a method for implanting a medical device comprising: (a) infiltrating a tissue of a host where the medical device is to be, or has been, implanted with a composition comprising an anti-fibrotic agent, an anti-infective agent and a polymer, and (b) implanting the medical device into the host.

For each afore stated aspect, one or more (e.g., any two) of the following features may be used to further define the invention in terms of the device used in the inventive method: the medical device is a pump adapted for delivering insulin; the medical device is a pump adapted for delivering a narcotic; the medical device is a pump adapted for delivering a chemotherapeutic agent; the medical device is a pump adapted for delivering an anti-arrhythmic drug; the medical device is a pump adapted for delivering an anti-spasmotic drug; the medical device is a pump adapted for delivering an anti-spastic agent; the medical device is a pump adapted for delivering an antibiotic; the medical device is a pump adapted for delivering a drug only when changes in the host are detected; the medical device is a pump adapted for delivering a drug as a continuous slow release; the medical device is a pump adapted for delivering a drug at prescribed dosages in a pulsatile manner; the medical device is a pump a programmable drug delivery pump; the medical device is a pump adapted for intraocularly delivering a drug; the medical device is a pump adapted for intrathecally delivering a drug; the medical device is a pump adapted for intraperitoneally delivering a drug; the medical device is a pump adapted for intra-arterially delivering a drug; the medical device is a pump adapted for intracardiac delivery of a drug; the medical device is an implantable osmotic pump; the medical device is an ocular drug delivery pump; the medical device is metering system; the medical device is a peristaltic (roller) pump; the medical device is an electronically driven pump; the medical device is an elastromeric pump; the medical device is a spring-contraction pump; the medical device is a gas-driven pump; the medical device is a hydraulic pump; the medical device is a piston-dependent pump; the medical device is a non-piston-dependent pump; the medical device is a dispensing chamber; the medical device is an infusion pump; and the medical device is a passive pump.

27. Soft Tissue Implant

In another aspect, the present invention provides a method for implanting a medical device comprising: (a) infiltrating a tissue of a host where the medical device is to be, or has been, implanted with i) an anti-fibrotic agent, ii) an anti-infective agent, iii) a polymer; iv) a composition comprising an anti-fibrotic agent and a polymer, v) a composition comprising an anti-infective agent and a polymer, or vi) a composition comprising an anti-fibrotic agent, an anti-infective agent and a polymer, and (b) implanting the medical device into the host, wherein the medical device is a soft tissue implant.

Optionally, in separate aspects, the invention provides: a method for implanting a medical device comprising: (a) infiltrating a tissue of a host where the medical device is to be, or has been, implanted with an anti-fibrotic agent, and (b) implanting the medical device into the host; a method for implanting a medical device comprising: (a) infiltrating a tissue of a host where the medical device is to be, or has been, implanted with an anti-infective agent, and (b) implanting the medical device into the host; a method for implanting a medical device comprising: (a) infiltrating a tissue of a host where the medical device is to be, or has been, implanted with a polymer; and (b) implanting the medical device into the host; a method for implanting a medical device comprising: (a) infiltrating a tissue of a host where the medical device is to be, or has been, implanted with a composition comprising an anti-fibrotic agent and a polymer, and (b) implanting the medical device into the host; a method for implanting a medical device comprising: (a) infiltrating a tissue of a host where the medical device is to be, or has been, implanted with a composition comprising an anti-infective agent and a polymer, and (b) implanting the medical device into the host; and a method for implanting a medical device comprising: (a) infiltrating a tissue of a host where the medical device is to be, or has been, implanted with a composition comprising an anti-fibrotic agent, an anti-infective agent and a polymer, and (b) implanting the medical device into the host.

For each afore stated aspect, one or more (e.g., any two) of the following features may be used to further define the invention in terms of the device used in the inventive method: the medical device is a cosmetic implant; the medical device is a reconstructive implant; the medical device is a breast implant; the medical device is a breast implant that comprises silicone; the medical device is a breast implant that comprises saline; the medical device is a facial implant; the medical device is a chin implant; the medical device is a mandibular implant; the medical device is a lip implant; the medical device is a nasal implant; the medical device is a cheek implant; the medical device is a pectoral implant; the medical device is a buttocks implant; the medical device is an autogenous tissue implant; the medical device is an autogenous tissue implant that comprises adipose tissue; the medical device is an autogenous tissue implant that comprises an autogenous fat implant; the medical device is an autogenous tissue implant that comprises a dermal implant; the medical device is an autogenous tissue implant that comprises a dermal plug; the medical device is an autogenous tissue implant that comprises a tissue plug; the medical device is an autogenous tissue implant that comprises a muscular tissue flap; the medical device is an autogenous tissue implant that comprises a pedicle flap; the medical device is an autogenous tissue implant that comprises a pedicle flap, wherein the pedicle flap is from the back, abdomen, buttocks, thigh, or groin; the medical device is an autogenous tissue implant that comprises a cell extraction implant; the medical device is an autogenous tissue implant that comprises a suspension of autologous dermal fibroblasts; the medical device is a tissue filler; and the medical device is a fat graft.

The present invention, in various aspects and embodiments, provides the following methods for preventing surgical adhesions:

In one aspect, the present invention provides a method for preventing surgical adhesions, comprising delivering a tissue-reactive polymeric composition to a site in need thereof to provide coated tissue, and delivering a fibrosis-inhibiting agent to the coated tissue.

In another aspect, the present invention provides a method of preventing surgical adhesions, comprising delivering a composition between a dural sleeve and paravertebral musculature in a patient post-laminectomy, where the composition prevents surgical adhesions.

In another aspect, the present invention provides a method of preventing surgical adhesions, comprising coating a spinal nerve at a laminectomy site in a patient in need thereof with a composition, where the composition prevents surgical adhesions.

In another aspect, the present invention provides a method of preventing surgical adhesions, comprising infiltrating a composition into tissue around a spinal nerve at a laminectomy site in a patient in need thereof, where the composition prevents surgical adhesions.

In another aspect, the present invention provides a method of preventing surgical adhesions, comprising delivering a composition to a site of a surgical, disc resection in a patient in need thereof, where the composition prevents surgical adhesions.

In another aspect, the present invention provides a method of preventing surgical adhesions, comprising delivering a composition to a site of a microdiscectomy in a patient in need thereof, where the composition prevents surgical adhesions.

In another aspect, the present invention provides a method of preventing surgical adhesions, comprising delivering a composition to a site of a neurosurgical (brain) procedure in a patient in need thereof, where the composition prevents surgical adhesions.

In another aspect, the present invention provides a method of preventing surgical adhesions, comprising infiltrating into a spinal surgical site of a patient in need thereof, a composition that prevents surgical adhesions.

In another aspect, the present invention provides a method of preventing surgical adhesions, comprising delivering a composition to epidural tissue in a patient in need thereof, where the composition prevents surgical adhesions.

In another aspect, the present invention provides a method of preventing surgical adhesions, comprising delivering a composition to dural tissue in a patient in need thereof, where the composition prevents surgical adhesions.

In another aspect, the present invention provides a method of preventing surgical adhesions, comprising delivering a composition to a gynecological site in a patient in need thereof, where the composition prevents surgical adhesions.

In another aspect, the present invention provides a method of preventing surgical adhesions, comprising delivering a composition to a tissue surface of the pelvic side wall in a patient in need thereof, where the composition prevents surgical adhesions.

In another aspect, the present invention provides a method of preventing surgical adhesions, comprising delivering a composition to a peritoneal cavity in a patient in need thereof, where the composition prevents surgical adhesions.

In another aspect, the present invention provides a method of preventing surgical adhesions, comprising delivering a composition to a pelvic cavity in a patient in need thereof, where the composition prevents surgical adhesions.

In another aspect, the present invention provides a method of preventing surgical adhesions, comprising delivering a composition to a site of a laparotomy in a patient in need thereof, where the composition prevents surgical adhesions.

In another aspect, the present invention provides a method of preventing surgical adhesions, comprising delivering a composition to a site of an endoscopic procedure in a patient in need thereof, where the composition prevents surgical adhesions.

In another aspect, the present invention provides a method of preventing surgical adhesions, comprising delivering a composition to a site of a hernia repair in a patient in need thereof, where the composition prevents surgical adhesions.

In another aspect, the present invention provides a method of preventing surgical adhesions, comprising delivering a composition to a site of cholecystectomy in a patient in need thereof, where the composition prevents surgical adhesions.

In another aspect, the present invention provides a method of preventing surgical adhesions, comprising delivering a composition to a site of a cardiac procedure in a patient in need thereof, where the composition prevents surgical adhesions.

In another aspect, the present invention provides a method of preventing surgical adhesions, comprising delivering a composition to a site of cardiac transplant surgery in a patient in need thereof, where the composition prevents surgical adhesions.

In another aspect, the present invention provides a method of preventing surgical adhesions, comprising delivering a composition to a site of cardiac vascular repair in a patient in need thereof, where the composition prevents surgical adhesions.

In another aspect, the present invention provides a method of preventing surgical adhesions, comprising delivering a composition to a site of a heart valve replacement in a patient in need thereof, where the composition prevents surgical adhesions.

In another aspect, the present invention provides a method of preventing pericardial surgical adhesions, comprising delivering a composition to a site of pericardial surgery in a patient in need thereof, where the composition prevents surgical adhesions.

In another aspect, the present invention provides a method of preventing surgical adhesions, comprising delivering a composition to a site of an orthopedic surgical procedure in a patient in need thereof, where the composition prevents surgical adhesions.

In another aspect, the present invention provides a method of preventing surgical adhesions, comprising delivering a composition to a site of a torn ligament in a patient in need thereof, where the composition prevents surgical adhesions.

In another aspect, the present invention provides a method of preventing surgical adhesions, comprising delivering a composition to a site of a joint injury in a patient in need thereof, where the composition prevents surgical adhesions.

In another aspect, the present invention provides a method of preventing surgical adhesions, comprising delivering a composition to a site of a tendon injury in a patient in need thereof, where the composition prevents surgical adhesions.

In another aspect, the present invention provides a method of preventing surgical adhesions, comprising delivering a composition to a site of a cartilage injury in a patient in need thereof, where the composition prevents surgical adhesions.

In another aspect, the present invention provides a method of preventing surgical adhesions, comprising delivering a composition to a site of a muscle injury in a patient in need thereof, where the composition prevents surgical adhesions.

In another aspect, the present invention provides a method of preventing surgical adhesions, comprising delivering a composition to a site of a nerve injury in a patient in need thereof, where the composition prevents surgical adhesions.

In another aspect, the present invention provides a method of preventing surgical adhesions, comprising delivering a composition to a site of a cosmetic surgical procedure in a patient in need thereof, where the composition prevents surgical adhesions.

In another aspect, the present invention provides a method of preventing surgical adhesions, comprising delivering a composition to a site of a reconstructive surgical procedure in a patient in need thereof, where the composition prevents surgical adhesions.

In another aspect, the present invention provides a method of preventing surgical adhesions, comprising delivering a composition to a site of a breast implant in a patient in need thereof, where the composition prevents surgical adhesions.

The methods of preventing surgical adhesions as described herein may be further defined by one, two or more of the following features: the composition is delivered in conjunction with the placement of a medical implant; the composition is delivered in conjunction with the placement of a medical implant, and the composition is placed on tissue adjacent to the medical implant; the composition is delivered in conjunction with the placement of a medical implant, and the composition is placed on the medical implant; the composition is delivered via an endoscope; the composition is delivered through a needle; the composition is delivered through a catheter; the composition is delivered at the time of a surgery; and the composition is delivered using fluoroscopic guidance.

The present invention, in various aspects and embodiments, also provides the following methods for treating inflammatory arthritis:

In one aspect, the present invention provides a method for treatment of inflammatory arthritis, comprising delivering to a patient in need thereof a therapeutic composition, the composition comprising a) a polymer and/or a compound that forms a polymer in situ and b) an anti-fibrotic agent.

In another aspect, the present invention provides a method for prevention of inflammatory arthritis, comprising delivering to a patient in need thereof a therapeutic composition, the composition comprising a polymer and an anti-fibrotic agent.

In another aspect, the present invention provides a method for treatment of osteoarthritis, comprising delivering to a patient in need thereof a therapeutic composition, the composition comprising a polymer and an anti-fibrotic agent.

In another aspect, the present invention provides a method for prevention of osteoarthritis, comprising delivering to a patient in need thereof a therapeutic composition, the composition comprising a polymer and an anti-fibrotic agent.

In another aspect, the present invention provides a method for treatment of primary osteoarthritis, comprising delivering to a patient in need thereof a therapeutic composition, the composition comprising a polymer and an anti-fibrotic agent.

In another aspect, the present invention provides a method for prevention of primary osteoarthritis, comprising delivering to a patient in need thereof a therapeutic composition, the composition comprising a polymer and an anti-fibrotic agent.

In another aspect, the present invention provides a method for treatment of secondary osteoarthritis, comprising delivering to a patient in need thereof a therapeutic composition, the composition comprising a polymer and an anti-fibrotic agent.

In another aspect, the present invention provides a method for prevention of secondary osteoarthritis, comprising delivering to a patient in need thereof a therapeutic composition, the composition comprising a polymer and an anti-fibrotic agent.

In another aspect, the present invention provides a method for treatment of rheumatoid arthritis, comprising delivering to a patient in need thereof a therapeutic composition, the composition comprising a polymer and an anti-fibrotic agent.

In another aspect, the present invention provides a method for prevention of rheumatoid arthritis, comprising delivering to a patient in need thereof a therapeutic composition, the composition comprising a polymer and an anti-fibrotic agent.

The methods of treating inflammatory arthritis described herein may be further defined by one, two or more of the following features: the composition is delivered intravenously; the composition is delivered orally; the composition is delivered by subcutaneous injection; the composition is delivered by intramuscular injection; and the composition is delivered intra-articularly.

The present invention, in various aspects and embodiments, provides the following methods for treating hypertrophic scars or keloids.

In one aspect, the present invention provides a method for treating a hypertrophic scar in a patient in need thereof, comprising delivering to the patient a) an anti-fibrotic agent or b) a composition comprising i) an anti-fibrotic agent and ii) a polymer and/or a compound that forms a polymer in situ.

In another aspect, the present invention provides a method for treating a keloid in a patient in need thereof, comprising delivering to the patient a) an anti-fibrotic agent or b) a composition comprising i) an anti-fibrotic agent and ii) a polymer and/or a compound that forms a polymer in situ.

In certain embodiments, the agent or composition is directly injected into the scar or keloid. In certain other embodiments, the agent or composition is topically applied to the scar or keloid.

The present invention, in various aspect and embodiments, also provides the following methods for reducing cartilage loss:

In one aspect, the present invention provides a method for reducing cartilage loss following an injury to a joint in a patient in need thereof, comprising delivering to the patient a) an anti-fibrotic agent or b) a composition comprising i) an anti-fibrotic agent and ii) a polymer and/or a compound that forms a polymer in situ.

In another aspect, the present invention provides a method for preventing cartilage loss following an injury to a joint in a patient in need thereof, comprising delivering to the patient a) an anti-fibrotic agent or b) a composition comprising i) an anti-fibrotic agent and ii) a polymer and/or a compound that forms a polymer in situ.

In another aspect, the present invention provides a method for reducing cartilage loss following a cruciate ligament tear in a patient in need thereof, comprising delivering to the patient a) an anti-fibrotic agent or b) a composition comprising i) an anti-fibrotic agent and ii) a polymer and/or a compound that forms a polymer in situ.

In another aspect, the present-invention provides a method for preventing cartilage loss following a cruciate ligament tear in a patient in need thereof, comprising delivering to the patient a) an anti-fibrotic agent or b) a composition comprising i) an anti-fibrotic agent and ii) a polymer and/or a compound that forms a polymer in situ.

In another aspect, the present invention provides a method for reducing cartilage loss following a meniscal tear in a patient in need thereof, comprising delivering to the patient a) an anti-fibrotic agent or b) a composition comprising i) an anti-fibrotic agent and ii) a polymer and/or a compound that forms a polymer in situ.

In another aspect, the present invention provides a method for preventing cartilage loss following a meniscal ligament tear in a patient in need thereof, comprising delivering to the patient a) an anti-fibrotic agent or b) a composition comprising i) an anti-fibrotic agent and ii) a polymer and/or a compound that forms a polymer in situ.

In certain embodiments, the agent or composition is delivered intra-articularly.

The present invention, in various aspects and embodiments, provides the following methods for treating vascular diseases:

In one aspect, the present invention provides a method for treating vascular disease in a patient in need thereof, comprising delivering to the patient a) an anti-fibrotic agent or b) a composition comprising i) an anti-fibrotic agent and ii) a polymer and/or a compound that forms a polymer in situ. In certain embodiments, the agent or composition is delivered perivascularly.

In another aspect, the present invention provides a method for treating stenosis in a patient in need thereof, comprising delivering to the patient a) an anti-fibrotic agent or b) a composition comprising i) an anti-fibrotic agent and ii) a polymer and/or a compound that forms a polymer in situ. In certain embodiments, the agent or-composition is delivered perivascularly.

In another aspect, the present invention provides a method for treating restenosis in a patient in need thereof, comprising delivering to the patient a) an anti-fibrotic agent or b) a composition comprising i) an anti-fibrotic agent and ii) a polymer and/or a compound that forms a polymer in situ. In certain embodiments, the agent or composition is delivered perivascularly.

In another aspect, the present invention provides a method for treating atherosclerosis in a patient in need thereof, comprising delivering to the patient a) an anti-fibrotic agent or b) a composition comprising i) an anti-fibrotic agent and ii) a polymer and/or a compound that forms a polymer in situ. In certain embodiments, the agent or composition is delivered perivascularly.

The present invention, in various aspects and embodiments, provides a composition comprising i) an anti-fibrotic agent and ii) a polymer or a compound that forms a polymer in situ.

Additional Features Related to Methods and Compositions

In addition, for each of the afore stated aspects, one or more (e.g., any two) of the following features may be used to further define the invention in terms of the anti-fibrotic agent, where these features may be combined with any one or more of the afore stated devices (e.g., an afore stated aspect further define by an afore stated device, and further defined as follows): the anti-fibrotic agent inhibits cell regeneration; the anti-fibrotic agent inhibits angiogenesis; the anti-fibrotic agent inhibits fibroblast migration; the anti-fibrotic agent inhibits fibroblast proliferation; the anti-fibrotic agent inhibits deposition of extracellular matrix; the anti-fibrotic agent inhibits tissue remodeling; the anti-fibrotic agent is an angiogenesis inhibitor; the anti-fibrotic agent is a 5-lipoxygenase inhibitor or antagonist; the anti-fibrotic agent is a chemokine receptor antagonist; the anti-fibrotic agent is a cell cycle inhibitor; the anti-fibrotic agent is a taxane; the anti-fibrotic agent is an anti-microtubule agent, the anti-fibrotic agent is paclitaxel; the anti-fibrotic agent is not paclitaxel; the anti-fibrotic agent is an analogue or derivative of paclitaxel; the anti-fibrotic agent is a vinca alkaloid; the anti-fibrotic agent is camptothecin or an analogue or derivative thereof; the anti-fibrotic agent is a podophyllotoxin; the anti-fibrotic agent is a podophyllotoxin, wherein the podophyllotoxin is etoposide or an analogue or derivative thereof; the anti-fibrotic agent is an anthracycline; the anti-fibrotic agent is an anthracycline, wherein the anthracycline is doxorubicin or an analogue or derivative thereof; the anti-fibrotic agent is an anthracycline, wherein the anthracycline is mitoxantrone or an analogue or derivative thereof; the anti-fibrotic agent is a platinum compound; the anti-fibrotic agent is a nitrosourea; the anti-fibrotic agent is a nitroimidazole; the anti-fibrotic agent is a folic acid antagonist; the anti-fibrotic agent is a cytidine analogue; the anti-fibrotic agent is a pyrimidine analogue; the anti-fibrotic agent is a fluoropyrimidine analogue; the anti-fibrotic agent is a purine analogue; the anti-fibrotic agent is a nitrogen mustard or an analogue or derivative thereof; the anti-fibrotic agent is a hydroxyurea; the anti-fibrotic agent is a mytomicin or an analogue or derivative thereof; the anti-fibrotic agent is an alkyl sulfonate; the anti-fibrotic agent is a benzamide or an analogue or derivative thereof; the anti-fibrotic agent is a nicotinamide or an analogue or derivative thereof; the anti-fibrotic agent is a halogenated sugar or an analogue or derivative thereof; the anti-fibrotic agent is a DNA alkylating agent; the anti-fibrotic agent is an anti-microtubule agent; the anti-fibrotic agent is a topoisomerase inhibitor; the anti-fibrotic agent is a DNA cleaving agent; the anti-fibrotic agent is an antimetabolite; the anti-fibrotic agent inhibits adenosine deaminase; the anti-fibrotic agent inhibits purine ring synthesis; the anti-fibrotic agent is a nucleotide interconversion inhibitor; the anti-fibrotic agent inhibits dihydrofolate reduction; the anti-fibrotic agent blocks thymidine monophosphate; the anti-fibrotic agent causes DNA damage; the anti-fibrotic agent is a DNA intercalation agent; the anti-fibrotic agent is a RNA synthesis inhibitor; the anti-fibrotic agent is a pyrimidine synthesis inhibitor; the anti-fibrotic agent inhibits ribonucleotide synthesis or function; the anti-fibrotic agent inhibits thymidine monophosphate synthesis or function; the anti-fibrotic agent inhibits DNA synthesis; the anti-fibrotic agent causes DNA adduct formation; the anti-fibrotic agent inhibits protein synthesis; the anti-fibrotic agent inhibits microtubule function; the anti-fibrotic agent is a cyclin dependent protein kinase inhibitor; the anti-fibrotic agent is an epidermal growth factor kinase inhibitor; the anti-fibrotic agent is an elastase inhibitor; the anti-fibrotic agent is a factor Xa inhibitor; the anti-fibrotic agent is a farnesyltransferase inhibitor; the anti-fibrotic agent is a fibrinogen antagonist; the anti-fibrotic agent is a guanylate cyclase stimulant; the anti-fibrotic agent is a heat shock protein 90 antagonist; the anti-fibrotic agent is a heat shock protein 90 antagonist, wherein the heat shock protein 90 antagonist is geldanamycin or an analogue or derivative thereof; the anti-fibrotic agent is a guanylate cyclase stimulant; the anti-fibrotic agent is a HMGCOA reductase inhibitor; the anti-fibrotic agent is a HMGCOA reductase inhibitor, wherein the HMGCOA reductase inhibitor is simvastatin or an analogue or derivative thereof; the anti-fibrotic agent is a hydroorotate dehydrogenase inhibitor; the anti-fibrotic agent is an IKK2 inhibitor; the anti-fibrotic agent is an IL-1 antagonist; the anti-fibrotic agent is an ICE antagonist; the anti-fibrotic agent is an IRAK antagonist; the anti-fibrotic agent is an IL-4 agonist; the anti-fibrotic agent is an immunomodulatory agent; the anti-fibrotic agent is sirolimus or an analogue or derivative thereof; the anti-fibrotic agent is not sirolimus; the anti-fibrotic agent is everolimus or an analogue or derivative thereof; the anti-fibrotic agent is tacrolimus or an analogue or derivative thereof; the anti-fibrotic agent is not tacrolimus; the anti-fibrotic agent is biolmus or an analogue or derivative thereof; the anti-fibrotic agent is tresperimus or an analogue or derivative thereof; the anti-fibrotic agent is auranofin or an analogue or derivative thereof; the anti-fibrotic agent is 27-0-demethylrapamycin or an analogue or derivative thereof; the anti-fibrotic agent is gusperimus or an analogue or derivative thereof; the anti-fibrotic agent is pimecrolimus or an analogue or derivative thereof; the anti-fibrotic agent is ABT-578 or an analogue or derivative thereof; the anti-fibrotic agent is an inosine monophosphate dehydrogenase (IMPDH) inhibitor; the anti-fibrotic agent is an IMPDH inhibitor, wherein the IMPDH inhibitor is mycophenolic acid or an analogue or derivative thereof; the anti-fibrotic agent is an IMPDH inhibitor, wherein the IMPDH inhibitor is 1-alpha-25 dihydroxy vitamin D₃ or an analogue or derivative thereof; the anti-fibrotic agent is a leukotriene inhibitor; the anti-fibrotic agent is a MCP-1 antagonist; the anti-fibrotic agent is a MMP inhibitor; the anti-fibrotic agent is an NF kappa B inhibitor; the anti-fibrotic agent is an NF kappa B inhibitor, wherein the NF kappa B inhibitor is Bay 11-7082; the anti-fibrotic agent is an NO antagonist; the anti-fibrotic agent is a p38 MAP kinase inhibitor; the anti-fibrotic agent is a p38 MAP kinase inhibitor, wherein the p38 MAP kinase inhibitor is SB 202190; the anti-fibrotic agent is a phosphodiesterase inhibitor; the anti-fibrotic agent is a TGF beta inhibitor; the anti-fibrotic agent is a thromboxane A2 antagonist; the anti-fibrotic agent is a TNF alpha antagonist; the anti-fibrotic agent is a TACE inhibitor; the anti-fibrotic agent is a tyrosine kinase inhibitor; the anti-fibrotic agent is a vitronectin inhibitor; the anti-fibrotic agent is a fibroblast growth factor inhibitor; the anti-fibrotic agent is a protein kinase inhibitor; the anti-fibrotic agent is a PDGF receptor kinase inhibitor; the anti-fibrotic agent is an endothelial growth factor receptor kinase inhibitor; the anti-fibrotic agent is a retinoic acid receptor antagonist; the anti-fibrotic agent is a platelet derived growth factor receptor kinase inhibitor; the anti-fibrotic agent is a fibrinogen antagonist; the anti-fibrotic agent is an antimycotic agent; the anti-fibrotic agent is an antimycotic agent, wherein the antimycotic agent is sulconizole; the anti-fibrotic agent is a bisphosphonate; the anti-fibrotic agent is a phospholipase A1 inhibitor; the anti-fibrotic agent is a histamine H1/H2/H3 receptor antagonist; the anti-fibrotic agent is a macrolide antibiotic; the anti-fibrotic agent is a GPIIb/IIIa receptor antagonist; the anti-fibrotic agent is an endothelin receptor antagonist; the anti-fibrotic agent is a peroxisome proliferator-activated receptor agonist; the anti-fibrotic agent is an estrogen receptor agent; the anti-fibrotic agent is a somastostatin analogue; the anti-fibrotic agent is a neurokinin 1 antagonist; the anti-fibrotic agent is a neurokinin 3 antagonist; the anti-fibrotic agent is a VLA-4 antagonist; the anti-fibrotic agent is an osteoclast inhibitor; the anti-fibrotic agent is a DNA topoisomerase ATP hydrolyzing inhibitor; the anti-fibrotic agent is an angiotensin I converting enzyme inhibitor; the anti-fibrotic agent is an angiotensin II antagonist; the anti-fibrotic agent is an enkephalinase inhibitor; the anti-fibrotic agent is a peroxisome proliferator-activated receptor gamma agonist insulin sensitizer; the anti-fibrotic agent is a protein kinase C inhibitor; the anti-fibrotic agent is a ROCK (rho-associated kinase) inhibitor; the anti-fibrotic agent is a CXCR3 inhibitor; the anti-fibrotic agent is an Itk inhibitor; the anti-fibrotic agent is a cytosolic phospholipase A₂-alpha inhibitor; the anti-fibrotic agent is a PPAR agonist; the anti-fibrotic agent is an immunosuppressant; the anti-fibrotic agent is an Erb inhibitor; the anti-fibrotic agent is an apoptosis agonist; the anti-fibrotic agent is a lipocortin agonist; the anti-fibrotic agent is a VCAM-1 antagonist; the anti-fibrotic agent is a collagen antagonist; the anti-fibrotic agent is an alpha 2 integrin antagonist; the anti-fibrotic agent is a TNF alpha inhibitor; the anti-fibrotic agent is a nitric oxide inhibito; the anti-fibrotic agent is a cathepsin inhibitor; the anti-fibrotic agent is not an anti-inflammatory agent; the anti-fibrotic agent is not a steroid; the anti-fibrotic agent is not a glucocorticosteroid; the anti-fibrotic agent is not dexamethasone; the anti-fibrotic agent is not beclomethasone; the anti-fibrotic agent is not dipropionate; the anti-fibrotic agent is not an anti-infective agent; the anti-fibrotic agent is not an antibiotic; and/or the anti-fibrotic agent is not an anti-fungal agent.

In addition, for each of the afore stated aspects, one or more (e.g., any two) of the following features may be used to further define the invention in terms of the anti-infective agent, where these features may be combined with any one or more of the afore stated devices (e.g., an afore stated aspect further define by an afore stated device, and further defined as follows): the anti-infective agent is an anthracycline; the anti-infective agent is doxorubicin; the anti-infective agent is mitoxantrone; the anti-infective agent is a fluoropyrimidine; the anti-infective agent is 5-fluorouracil (5-FU); the anti-infective agent is a folic acid antagonist; the anti-infective agent is methotrexate; the anti-infective agent is a podophylotoxin; the anti-infective agent is etoposide; the anti-infective agent is camptothecin; the anti-infective agent is a hydroxyurea; the anti-infective agent is a platinum complex; and/or the anti-infective agent is cisplatin. The compositions may further optionally comprise an anti-thrombotic agent.

In addition, for each of the afore stated aspects, one or more (e.g., any two) of the following features may be used to further define the invention in terms of the polymer, where any one or more of these features may be combined with any one or more of the afore stated devices, anti-fibrotic agents and anti-infective agents (e.g., an afore stated aspect further define by a particular device and a particular anti-fibrotic agent, further defined as follows); the polymer is formed from reactants comprising a naturally occurring polymer; the polymer is formed from reactants comprising protein; the polymer is formed from reactants comprising carbohydrate; the polymer is formed from reactants comprising biodegradable polymer; the polymer is formed from reactants comprising nonbiodegradable polymer; the polymer is formed from reactants comprising collagen; the polymer is formed from reactants comprising methylated collagen; the polymer is formed from reactants comprising fibrinogen; the polymer is formed from reactants comprising thrombin; the polymer is formed from reactants comprising blood plasma; the polymer is formed from reactants comprising calcium salt; the polymer is formed from reactants comprising an antifibronolytic agent; the polymer is formed from reactants comprising fibrinogen analog; the polymer is formed from reactants comprising albumin; the polymer is formed from reactants comprising plasminogen; the polymer is formed from reactants comprising von Willebrands factor; the polymer is formed from reactants comprising Factor VIII; the polymer is formed from reactants comprising hypoallergenic collagen; the polymer is formed from reactants comprising atelopeptidic collagen; the polymer is formed from reactants comprising telopeptide collagen; the polymer is formed from reactants comprising crosslinked collagen; the polymer is formed from reactants comprising aprotinin; the polymer is formed from reactants comprising epsilon-amino-n-caproic acid; the polymer is formed from reactants comprising gelatin; the polymer is formed from reactants comprising protein conjugates; the polymer is formed from reactants comprising gelatin conjugates; the polymer is formed from reactants comprising a synthetic polymer; the polymer is formed from reactants comprising a synthetic isocyanate-containing compound; the polymer is formed from reactants comprising a synthetic thiol-containing compound; the polymer is formed from reactants comprising a synthetic compound containing at least two thiol groups; the polymer is formed from reactants comprising a synthetic compound containing at least three thiol groups; the polymer is formed from reactants comprising a synthetic compound containing at least four thiol groups; the polymer is formed from reactants comprising a synthetic amino-containing compound; the polymer is formed from reactants comprising a synthetic compound containing at least two amino groups; the polymer is formed from reactants comprising a synthetic compound containing at least three amino groups; the polymer is formed from reactants comprising a synthetic compound containing at least four amino groups; the polymer is formed from reactants comprising a synthetic compound comprising a carbonyl-oxygen-succinimidyl group; the polymer is formed from reactants comprising a synthetic compound comprising at least two carbonyl-oxygen-succinimidyl groups; the polymer is formed from reactants comprising a synthetic compound comprising at least three carbonyl-oxygen-succinimidyl groups; the polymer is formed from reactants comprising a synthetic compound comprising at least four carbonyl-oxygen-succinimidyl groups; the polymer is formed from reactants comprising a synthetic polyalkylene oxide-containing compound; the polymer is formed from reactants comprising a synthetic compound comprising both polyalkylene oxide and biodegradable polyester blocks; the polymer is formed from reactants comprising a synthetic polyalkylene oxide-containing compound having reactive amino groups; the polymer is formed from reactants comprising a synthetic polyalkylene oxide-containing compound having reactive thiol groups; the polymer is formed from reactants comprising a synthetic polyalkylene oxide-containing compound having reactive carbonyl-oxygen-succinimidyl groups; the polymer is formed from reactants comprising a synthetic compound comprising a biodegradable polyester block; the polymer is formed from reactants comprising a synthetic polymer formed in whole or part from lactic acid or lactide; the polymer is formed from reactants comprising a synthetic polymer formed in whole or part from glycolic acid or glycolide; the polymer is formed from reactants comprising polylysine; the polymer is formed from reactants comprising (a) protein and (b) a compound comprising a polyalkylene oxide portion; the polymer is formed from reactants comprising (a) protein and (b) polylysine; the polymer is formed from reactants comprising (a) protein and (b) a compound having at least four thiol groups; the polymer is formed from reactants comprising (a) protein and (b) a compound having at least four amino groups; the polymer is formed from reactants comprising (a) protein and (b) a compound having at least four carbonyl-oxygen-succinimide groups; the polymer is formed from reactants comprising (a) protein and (b) a compound having a biodegradable region formed from reactants selected from lactic acid, lactide, glycolic acid, glycolide, and epison-caprolactone; the polymer is formed from reactants comprising (a) collagen and (b) a compound comprising a polyalkylene oxide portion; the polymer is formed from reactants comprising (a) collagen and (b) polylysine; the polymer is formed from reactants comprising (a) collagen and (b) a compound having at least four thiol groups; the polymer is formed from reactants comprising (a) collagen and (b) a compound having at least four amino groups; the polymer is formed from reactants comprising (a) collagen and (b) a compound having at least four carbonyl-oxygen-succinimide groups; the polymer is formed from reactants comprising (a) collagen and (b) a compound having a biodegradable region formed from reactants selected from lactic acid, lactide, glycolic acid, glycolide, and epison-caprolactone; the polymer is formed from reactants comprising (a) methylated collagen and (b) a compound comprising a polyalkylene oxide portion; the polymer is formed from reactants comprising (a) methylated collagen and (b) polylysine; the polymer is formed from reactants comprising (a) methylated collagen and (b) a compound having at least four thiol groups; the polymer is formed from reactants comprising (a) methylated collagen and (b) a compound having at least four amino groups; the polymer is formed from reactants comprising (a) methylated collagen and (b) a compound having at least four carbonyl-oxygen-succinimide groups; the polymer is formed from reactants comprising (a) methylated collagen and (b) a compound having a biodegradable region formed from reactants selected from lactic acid, lactide, glycolic acid, glycolide, and epison-caprolactone; the polymer is formed from reactants comprising hyaluronic acid; the polymer is formed from reactants comprising a hyaluronic acid derivative; the polymer is formed from reactants comprising pentaerythritol poly(ethylene glycol)ether tetra-sulfhydryl of number average molecular weight between 3,000 and 30,000; the polymer is formed from reactants comprising pentaerythritol poly(ethylene glycol)ether tetra-amino of number average molecular weight between 3,000 and 30,000; the polymer is formed from reactants comprising (a) a synthetic compound having a number average molecular weight between 3,000 and 30,000 and comprising a polyalkylene oxide region and multiple nucleophilic groups, and (b) a synthetic compound having a number average molecular weight between 3,000 and 30,000 and comprising a polyalkylene oxide region and multiple electrophilic groups; the composition comprises a colorant; and the composition is sterile.

The following examples are offered by way of illustration, and not by way of limitation.

### EXAMPLES

### Example 1

### Preparation of Drug Loaded Microspheres by Spray Drying

3.6 grams of methoxy poly(ethylene glycol 5000))-block-(poly (DL-lactide). (65:35 MePEG:PDLLA weight ratio) was dissolved in 200 ml methylene chloride. 400 mg of a drug (mycophenolic acid (MPA), chlorpromazine (CPZ) or paclitaxel (PTX)) was added and the resulting solution was spray dried. Inlet temperature 50° C., outlet temperature <39° C., aspirator 100%, flow rate 700 l/hr. The collected microspheres were dried under vacuum at room temperature overnight to produce uniform, spherical particles having size ranges of less than about 10 microns (typically about 0.5 to about 2 microns).

### Example 2

### MPA Loaded Microspheres (<10 Micron) by the W/O/W Emulsion Process

100 ml of freshly prepared 10% polyvinyl alcohol (PVA) solution and 10 ml of pH 3 acetic acid solution saturated with MPA was added into a 600 ml beaker. The acidified PVA solution was stirred at 2000 rpm for 30 minutes. Meanwhile, a solution of 400 mg MPA and 800 mg MePEG5000-PDLLA (65:35) in 20 ml dichloromethane was prepared. The polymer/dichloromethane solution was added dropwise to the PVA solution while stirring at 2000 rpm with a Fisher DYNA-MIX stirrer. After addition was complete, the solution was allowed to stir for an additional 45 minutes. The microsphere solution was transferred to several disposable graduated polypropylene conical centrifuge tubes, washed with pH 3 acetic acid solution saturated with MPA, and centrifuged at 2600 rpm for 10 minutes. The aqueous layer was decanted and the washing, centrifuging and decanting was repeated 3 times. The combined, washed microspheres were freeze-dried and vacuum dried to remove any excess water.

### Example 3

### MPA Containing Microspheres (50-100 Micron) by the W/O/W Emulsion Process

Microspheres having an average size of about 50-100 microns were prepared using a 1 % PVA solution and 500 rpm stirring rate using the same procedure described in Example 2.

### Example 4

### CPZ and PTX Containing Microspheres by the W/O/W Emulsion Process

PTX and CPZ containing microspheres were prepared using the procedure described in Example 2 with the exception that the PVA solution and the washing solution does not have to be acidified and saturated with the drug.

### Example 5

### Paclitaxel Containing Micelles

MePEG2000 (41 g) and MePEG2000-PDLLA (60:40) (410 g) were combined in a vessel and heated to 75° C. with stirring. After the polymers were completely melted and mixed, the temperature was decreased to 55° C. Meanwhile, a PTX solution in tetrahydrofuran (46 g/200 ml) was prepared and poured into the polymer solution under constant stirring. Stirring was continued for and additional hour. The PTX containing micelles were dried at 50° C. under vacuum to remove solvent and were ground on a 2 mm mesh screen after cooling.

### Example 6

### Tetra Functional Poly(Ethylene Glycol)Succinimidyl Glutarate, (PEG-SG4), Non-Gelling Formulation

A 1 ml syringe (syringe 1) equipped with a luer-lock mixing connector was filled with PEG-SG4 (100 mg) (Sunbio, Inc., Orinda, Calif.). A 1 ml capped syringe (syringe 2) was filled with 0.25 ml of 6.3 mM HCl solution (pH 2.1). A 1 ml capped syringe (syringe 3) was filled with 0.25 ml 0.12 M monobasic sodium phosphate and 0.2 M sodium carbonate (pH 9.7) buffer. The solid contents of syringe 1 and the acidic solution of syringe 2 were mixed through a mixing connector by repeatedly transferring the contents from one syringe to the other. After complete mixing, the entire mixture was pushed into one of the syringes. The syringe containing the mixture then was attached to one inlet of an applicator (MICROMEDICS air assisted spray-applicator (Model SA-6105)). Syringe 3 containing the pH 9.7 solution was attached onto another inlet of the applicator. The formulation was applied to a tissue surface as specified by the applicator manufacturer.

### Example 7

### Gelling Formulation (Premix) I

A 1 ml syringe (syringe 1) equipped with a luer-lock mixing connector was filled with a mixture of PEG-SG4 (50 mg) and PEG-SH4 (tetra functional poly (ethylene glycol) thiol) (50 mg) (Sunbio, Inc.) (referred to as “premix”). A 1 ml capped syringe (syringe 2) was filled with 0.25 ml of 6.3 mM HCl solution (pH 2.1). A 1 ml capped syringe (syringe 3) was filled with 0.25 ml 0.12 M monobasic sodium phosphate and 0.2 M sodium carbonate (pH 9.7) buffer. The components were are mixed and applied to a tissue surface using the procedure described in Example 6.

### Example 8

### Tetra Functional Poly(Ethylene Glycol)Amine, (PEG-N4) Gelling Formulation

A 1 ml syringe (syringe 1) equipped with a luer-lock mixing connector was filled with PEG-SG4 (50 mg) and PEG-SH4 (tetra functional poly (ethylene glycol) thiol) (10, 25 or 40 mg). A 1 ml capped syringe (syringe 2) was filled with 0.25 ml of 6.3 mM HCl solution (pH 2.1). A 1 ml capped syringe (syringe 3) was filled with 0.25 ml 0.12 M monobasic sodium phosphate and 0.2 M sodium carbonate (pH 9.7) buffer. A 1 ml syringe (syringe 4) equipped with luer-lock mixing connector was filled with PEG-N4 (Sunbio, Inc.) (40, 25 or 10 mg) to make a mixture (50 mg total) of PEG-SH4 (in syringe 1) and PEG-N4 (in syringe 4). The contents of syringe 1 and syringe 2 were mixed through the mixing connector by repeatedly transferring the contents from one syringe to the other. After complete mixing, all of the formulation was pushed into one of the syringes which was then attached to one inlet of an applicator (MICROMEDICS air assisted spray-applicator (Model SA-6105)). Syringe 4 was attached to syringe 3 containing the pH 9.7 solution with a mixing connector. After complete mixing of the contents of syringe 3 and 4, the mixture was pushed into one of the syringes, which was then attached onto a second inlet of the applicator. The formulation was applied to a tissue surface as specified by the applicator manufacturer.

### Example 9

### Mycophenolic Acid and Disodium Salt of MPA (NA₂MPA): in PEG-SG4

Preparation of disodium salt of MPA (Na₂MPA): Na₂MPA was prepared by dissolving MPA (1, 10, or 100 g) in IPA (44 ml, 440 ml, or 4.4 L, respectively) 2 molar equivalents of 1M NaOH (aq) were quickly added to the solution with vigorous stirring. The resulting slurry was then brought to a boil until a clear yellow solution resulted. Stirring was ceased and the solution was allowed to cool to room temperature. The resulting cake of crystals were mobilized mechanically, filtered, washed with copious IPA, and dried under vacuum to yield white, highly crystalline fibers of Na₂MPA (yields typically 70-80%).

A 1 ml syringe (syringe 1) equipped with a luer-lock mixing connector was filled with PEG-SG4 (100 mg). A 1 ml capped syringe (syringe 2) was filled with 0.25 ml of 6.3 mM HCl solution (pH 2.1). A 1 ml capped syringe (syringe 3) was filled with 0.25 ml 0.12 M monobasic sodium phosphate and 0.2 M sodium carbonate (pH 9.7) buffer. A 1 ml syringe (syringe 4) equipped with luer-lock mixing connector was filled with MPA (5 mg) and Na₂MPA (95 mg), both sifted <100 micron. The contents of syringe 4 and syringe 2 were mixed through a mixing connector by repeatedly transferring the contents from one syringe to the other. This solution was then used to reconstitute the solids in syringe 1. After complete mixing, all of the formulation was pushed into one of the syringes which was then attached to one inlet of an applicator (MICROMEDICS air assisted spray-applicator (Model SA-6105)). Syringe 3 containing the pH 9.7 solution was attached onto the other inlet of the applicator. The formulation was applied to a tissue surface as specified by the applicator manufacturer.

### Example 10

### Mycophenolic Acid in Premix

A 1 ml syringe (syringe 1) equipped with a luer-lock mixing connector was filled with a mixture of PEG-SH4 (50 mg), PEG-SG4 (50 mg), and MPA (100 mg, sifted <100 micron). A 1 ml capped syringe (syringe 2) was filled with 0.25 ml of 6.3mM HCl solution (pH 2.1). A 1 ml capped syringe (syringe 3) was filled with 0.35 ml 0.24 M monobasic sodium phosphate and 0.4 M sodium carbonate (pH 10.0) buffer. The components were mixed and applied to a tissue surface using the procedure described in Example 6.

### Example 11

### Mycophenolic Acid and Disodium Salt of MPA (NA₂MPA) in Premix

A 1 ml syringe (syringe 1) equipped with a luer-lock mixing connector was filled with a mixture of PEG-SG4 (50 mg) and PEG-SH4 (50 mg). A 1 ml capped syringe (syringe 2) was filled with 0.25 ml of 6.3 mM HCl solution (pH 2.1). A 1 ml capped syringe (syringe 3) was filled with 0.25 ml 0.12 M monobasic sodium phosphate and 0.2 M sodium carbonate (pH 9.7) buffer. A 1 ml syringe (syringe 4) equipped with luer-lock mixing connector was filled with MPA (5 mg) and Na₂MPA (95 mg), both sifted <100 micron. The components were mixed and applied to a tissue surface using the procedure described in Example 9.

### Example 12

### Chlorpromazine in Premix

A 1 ml syringe (syringe 1) equipped with a luer-lock mixing connector was filled with a mixture of PEG-SG4 (50 mg), PEG-SH4 (50 mg), and CPZ (5 or 10 mg). A 1 ml capped syringe (syringe 2) was filled with 0.25 ml of 6.3 mM HCl solution (pH 2.1). A 1 ml capped syringe (syringe 3) was filled 0.25 ml 0.12 M monobasic sodium phosphate and 0.2 M sodium carbonate (pH 9.7) buffer. The components were mixed and applied to a tissue surface using the procedure described in Example 6.

### Example 13

### Paclitaxel Loaded Microspheres in Premix

A 1 ml syringe (syringe 1) equipped with a luer-lock mixing connector was filled with a mixture of PEG-SG4 (50 mg), PEG-SH4 (50 mg), and 10% PTX loaded MePEG5000-PDLLA (65:35) microspheres prepared by spray drying (0.5 or 2 mg) (prepared using the procedure described in Example 17). A 1 ml capped syringe (syringe 2) was filled with 0.25 ml of 6.3 mM HCl solution (pH 2.1). A 1 ml capped syringe (syringe 3) was filled 0.25 ml 0.12 M monobasic sodium phosphate and 0.2 M sodium carbonate (pH 9.7) buffer. The components were mixed and applied to a tissue surface using the procedure described in Example 1.

### Example 14

### CPZ Loaded Microspheres in Premix

A 1 ml syringe (syringe 1) equipped with a luer-lock mixing connector was filled with a mixture of PEG-SG4 (50 mg), PEG-SH4 (50 mg), and 10% CPZ loaded MePEG5000-PDLLA (65:35) microspheres prepared by spray drying (50 or 100 mg) (prepared using the procedure described in Example 17). A 1 ml capped syringe (syringe 2) was filled with 0.25 ml of 6.3 mM HCl solution (pH 2.1). A 1 ml capped syringe (syringe 3) was filled 0.25 ml 0.12 M monobasic sodium phosphate and 0.2 M sodium carbonate (pH 9.7) buffer. The components were mixed and applied to a tissue surface using the procedure described in Example 6.

### Example 15

### MPA Loaded Microspheres in Premix

A 1 ml syringe (syringe 1) equipped with a luer-lock mixing connector was filled with a mixture of PEG-SG4 (50 mg), PEG-SH4 (50 mg), and 10% MPA loaded MePEG5000-PDLLA 65:35 microspheres prepared by spray drying (25 or 75 mg) (prepared using the procedure described in Example 17). A 1 ml capped syringe (syringe 2) was filled with 0.25 ml 6.3 mM HCl solution (pH 2.1). A 1 ml capped syringe (syringe 3) was filled 0.35 ml 0.24 M monobasic sodium phosphate and 0.4 M sodium carbonate (pH 10.0) buffer. The components were mixed and applied to a tissue surface using the procedure described in Example 6.

### Example 16

### Incorporation of PTX Loaded Micelles into Premix

A 1 ml syringe (syringe 1) equipped with a luer-lock mixing connector was filled with a mixture of PEG-SG4 (50 mg) and PEG-SH4 (50 mg). A 2 ml serum vial was filled with 1.5 ml of 6.3 mM HCl solution (pH 2.1). A 1 ml capped syringe (syringe 2) was filled with 0.25 ml 0.12 M monobasic sodium phosphate and 0.2 M sodium carbonate (pH 9.7) buffer. A 2 ml serum vial was filled with 10% PTX loaded micelles (2 mg or 8 mg) (prepared as in Example 21) and reconstituted with 1 ml of the pH 2.1 solution. 0.25 ml of the micelle solution was removed with a 1 ml syringe; the syringe was attached to syringe 1 containing the solids PEG-SG4 and PEG-SH4; and the components were mixed through the mixing connector by repeatedly transferring the contents from one syringe to the other. After complete mixing, the entire mixture was pushed into one of the syringes, which was then attached to one inlet of an applicator (MICROMEDICS air assisted spray-applicator (Model SA-6105)). Syringe 3 containing the pH 9.7 solution was attached onto the other inlet of the applicator. The formulation was applied to a tissue surface as specified by the applicator manufacturer.

### Example 17

A 3 ml syringe (syringe 1) equipped with a luer-lock mixing connector was filled with containing PEG-SG4 (400 mg). A 3 ml capped syringe (syringe 2) was filled with 1.0 ml of 6.3 mM HCl solution (pH 2.1). A 3 ml capped syringe (syringe 3) was filled 1 ml 0.12 M monobasic sodium phosphate and 0.2 M sodium carbonate (pH 9.7) buffer. The components were mixed and applied to a tissue surface using the procedure described in Example 6.

### Example 18

### Gelling Formulation (Premix) II

A 3 ml syringe (syringe 1) equipped with a luer-lock mixing connector was filled with a mixture of PEG-SG4 (200 mg) and PEG-SH4 (200 mg). A 3 ml capped syringe (syringe 2) was filled with 1.0 ml of 6.3 mM HCl solution (pH 2.1). A 3 ml capped syringe (syringe 3) was filled 1 ml 0.12 M monobasic sodium phosphate and 0.2 M sodium carbonate (pH 9.7) buffer. The components were mixed and applied to a tissue surface using the procedure described in Example 6.

### Example 19

### MPA Loaded Premix

A 3 ml syringe (syringe 1) equipped with a luer-lock mixing connector was filled with a mixture of PEG-SG4 (200 mg), PEG-SH4 (200 mg), and MPA (200 mg or 400 mg). A 3 ml capped syringe (syringe 2) was filled with 1 ml of 6.3 mM HCl solution (pH 2.1). A 3 ml capped syringe (syringe 3) was filled 1.5 ml 0.24 M monobasic sodium phosphate and 0.4 M sodium carbonate (pH 10) buffer. The components were mixed and applied to a tissue surface using the procedure described in Example 6.

### Example 20

### Screening Assay for Assessing the Effect of Various Compounds on Nitric Oxide Production by Macrophages

The murine macrophage cell line RAW 264.7 was trypsinized to remove cells from flasks and plated in individual wells of a 6-well plate. Approximately 2×10⁶ cells were plated in 2 ml of media containing 5% heat-inactivated fetal bovine serum (FBS). RAW 264.7 cells were incubated at 37° C. for 1.5 hours to allow adherence to plastic. Mitoxantrone was prepared in DMSO at a concentration of 10⁻² M and serially diluted 10-fold to give a range of stock concentrations (10⁻⁸ M to 10⁻² M). Media was then removed and cells were incubated in 1 ng/ml of recombinant murine IFNγ and 5 ng/ml of LPS with or without mitoxantrone in fresh media containing 5% FBS. Mitoxantrone was added to cells by directly adding mitoxantrone DMSO stock solutions, prepared earlier, at a 1/1000 dilution, to each well. Plates containing IFNγ, LPS plus or minus mitoxantrone were incubated at 37° C. for 24 hours (Chem. Ber. (1879) 12: 426; J. AOAC (1977) 60-594; Ann. Rev. Biochem. (1994) 63:175).

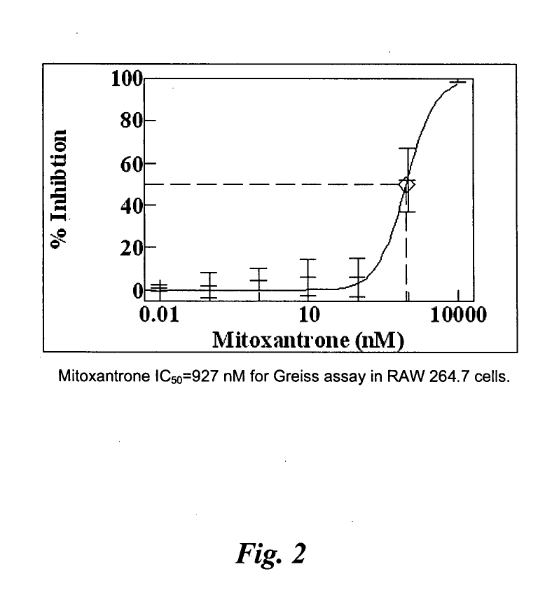

At the end of the 24 hour period, supernatants were collected from the cells and assayed for the production of nitrites. Each sample was tested in triplicate by aliquoting 50 μL of supernatant in a 96-well plate and adding 50 μL of Greiss Reagent A (0.5 g sulfanilamide, 1.5 ml H₃PO₄, 48.5 ml ddH₂O) and 50 μL of Greiss Reagent B (0.05 g N-(1-naphthyl)-ethylenediamine, 1.5 ml H₃PO₄, 48.5 ml ddH₂O). Optical density was read immediately on microplate spectrophotometer at 562 nm absorbance. Absorbance over triplicate wells was averaged after subtracting background and concentration values were obtained from the nitrite standard curve (1 μM to 2 mM). Inhibitory concentration of 50% (IC₅₀) was determined by comparing average nitrite concentration to the positive control (cell stimulated with IFNγ and LPS). An average of n=4 replicate experiments was used to determine IC₅₀ values for mitoxantrone (see, FIG. 2 (IC₅₀=927 nM)). The IC₅₀ values for the following additional compounds were determined using this assay: IC₅₀ (nM): paclitaxel, 7; CNI-1493, 249; halofuginone, 12; geldanamycin, 51; anisomycin, 68; 17-AAG, 840; epirubicin hydrochloride, 769.

### Example 21

### Screening Assay for Assessing the Effect of Various Anti-Scarring Agents on TNF-Alpha Production by Macrophages

The human macrophage cell line, THP-1 was plated in a 12 well plate such that each well contains 1×10⁶ cells in 2 ml of media containing 10% FCS. Opsonized zymosan was prepared by resuspending 20 mg of zymosan A in 2 ml of ddH₂O and homogenizing until a uniform suspension was obtained. Homogenized zymosan was pelleted at 250 g and resuspended in 4 ml of human serum for a final concentration of 5 mg/ml and incubated in a 37° C. water bath for 20 minutes to enable opsonization. Bay 11-7082 was prepared in DMSO at a concentration of 10⁻² M and serially diluted 10-fold to give a range of stock concentrations (10⁻⁸ M to 10⁻² M) (J. Immunol. (2000) 165: 411-418; J. Immunol. (2000) 164: 4804-4811; J. Immunol Meth. (2000) 235 (1-2): 33-40).

THP-1 cells were stimulated to produce TNFα by the addition of 1 mg/ml opsonized zymosan. Bay 11-7082 was added to THP-1 cells by directly adding DMSO stock solutions, prepared earlier, at a 1/1000 dilution, to each well. Each drug concentration was tested in triplicate wells. Plates were incubated at 37° C. for 24 hours.

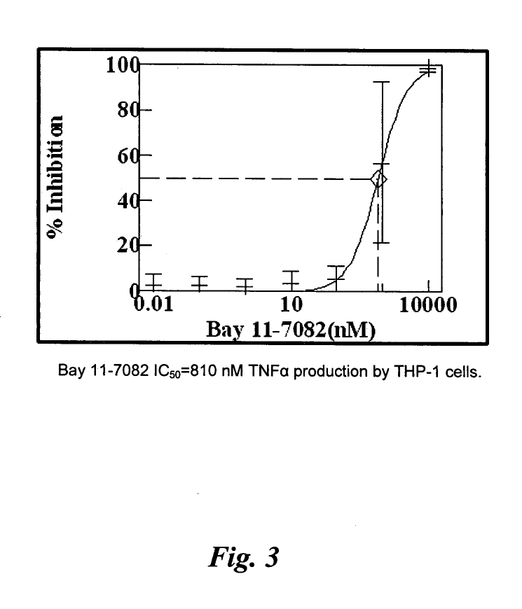

After a 24 hour stimulation, supernatants were collected to quantify TNFα production. TNFα concentrations in the supernatants were determined by ELISA using recombinant human TNFα to obtain a standard curve. A 96-well MaxiSorb plate was coated with 100 μL of anti-human TNFA Capture Antibody diluted in Coating Buffer (0.1 M sodium carbonate pH 9.5) overnight at 4° C. The dilution of Capture Antibody used was lot-specific and was determined empirically. Capture antibody was then aspirated and the plate washed 3 times with Wash Buffer (PBS, 0.05% TWEEN-20). Plates were blocked for 1 hour at room temperature with 200 μL/well of Assay Diluent (PBS, 10% FCS pH 7.0). After blocking, plates were washed 3 times with Wash Buffer. Standards and sample dilutions were prepared as follows: (a) sample supernatants were diluted ⅛ and 1/16; (b) recombinant human TNFα was prepared at 500 pg/ml and serially diluted to yield as standard curve of 7.8 pg/mi to 500 pg/ml. Sample supernatants and standards were assayed in triplicate and were incubated at room temperature for 2 hours after addition to the plate coated with Capture Antibody. The plates were washed 5 times and incubated with 100 μL of Working Detector (biotinylated anti-human TNFα detection antibody+avidin-HRP) for 1 hour at room temperature. Following this incubation, the plates were washed 7 times and 100 μL of Substrate Solution (tetramethylbenzidine, H₂O₂) was added to plates and incubated for 30 minutes at room temperature. Stop Solution (2 N H₂SO₄) was then added to the wells and a yellow color reaction was read at 450 nm with A correction at 570 nm. Mean absorbance was determined from triplicate data readings and the mean background was subtracted. TNFα concentration values were obtained from the standard curve. Inhibitory concentration of 50% (IC₅₀) was determined by comparing average TNFα concentration to the positive control (THP-1 cells stimulated with opsonized zymosan). An average of n=4 replicate experiments was used to determine IC₅₀ values for Bay 11-7082 (see FIG. 3; IC₅₀=810 nM)) and rapamycin (IC₅₀=51 nM; FIG. 4). The IC₅₀ values for the following additional compounds were determined using this assay: IC₅₀ (nM): geldanamycin, 14; mycophenolic acid, 756; mofetil, 792; chlorpromazine, 6; CNI-1493, 0.15; SKF 86002, 831; 15-deoxy prostaglandin J2, 742; fascaplycin, 701; podophyllotoxin, 75; mithramycin, 570; daunorubicin, 195; celastrol, 87; chromomycin A3, 394; vinorelbine, 605; vinblastine, 65.

### Example 22

### Surgical Adhesions Model to Assess Fibrosis Inhibiting Agents in Rats

The rat caecal sidewall model is used to as to assess the anti-fibrotic capacity of formulations in vivo. Sprague Dawley rats are anesthetized with halothane. Using aseptic precautions, the abdomen is opened via a midline incision. The caecum is exposed and lifted out of the abdominal cavity. Dorsal and ventral aspects of the caecum are successively scraped a total of 45 times over the terminal 1.5 cm using a #10 scalpel blade. Blade angle and pressure are controlled to produce punctate bleeding while avoiding severe tissue damage. The left side of the abdomen is retracted and everted to expose a section of the peritoneal wall that lies proximal to the caecum. The superficial layer of muscle (transverses abdominis) is excised over an area of 1×2 cm², leaving behind torn fibers from the second layer of muscle (internal oblique muscle). Abraded surfaces are tamponaded until bleeding stops. The abraded caecum is then positioned over the sidewall wound and attached by two sutures. The formulation is applied over both sides of the abraded caecum and over the abraded peritoneal sidewall. A further two sutures are placed to attach the caecum to the injured sidewall by a total of 4 sutures and the abdominal incision is closed in two layers. After 7 days, animals are evaluated post mortem with the extent and severity of adhesions being scored both quantitatively and qualitatively.

### Example 23

### Surgical Adhesions Model to Assess Fibrosis Inhibiting Agents in Rabbits

The rabbit uterine horn model is used to assess the anti-fibrotic capacity of formulations in vivo. Mature New Zealand White (NZW) female rabbits are placed under general anesthetic. Using aseptic precautions, the abdomen is opened in two layers at the midline to expose the uterus. Both uterine horns are lifted out of the abdominal cavity and assessed for size on the French Scale of catheters. Horns between #8 and #14 on the French Scale (2.5-4.5 mm diameter) are deemed suitable for this model. Both uterine horns and the opposing peritoneal wall are abraded with a #10 scalpel blade at a 45° angle over an area 2.5 cm in length and 0.4 cm in width until punctuate bleeding is observed. Abraded surfaces are tamponaded until bleeding stops. The individual horns are then opposed to the peritoneal wall and secured by two sutures placed 2 mm beyond the edges of the abraded area. The formulation is applied and the abdomen is closed in three layers. After 14 days, animals are evaluated post mortem with the extent and severity of adhesions being scored both quantitatively and qualitatively.

### Example 24

### Screening Assay for Assessing the Effect of Various Compounds on Cell Proliferation

Fibroblasts at 70-90% confluency were trypsinized, replated at 600 cells/well in media in 96-well plates and allowed to attach overnight. Mitoxantrone was prepared in DMSO at a concentration of 10⁻² M and diluted 10-fold to give a range of stock concentrations (10⁻⁸ M to 10⁻² M). Drug dilutions were diluted 1/1000 in media and added to cells to give a total volume of 200 μL/well. Each drug concentration was tested in triplicate wells. Plates containing fibroblasts and mitoxantrone were incubated at 37° C. for 72 hours (In vitro toxicol. (1990) 3: 219; Biotech. Histochem. (1993) 68: 29; Anal. Biochem. (1993) 213: 426).

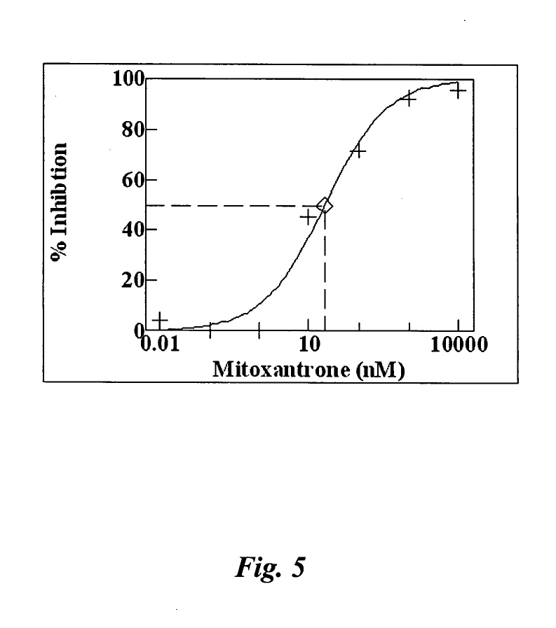

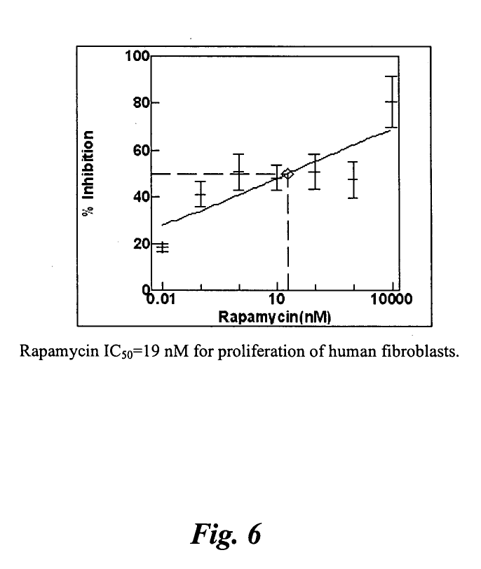

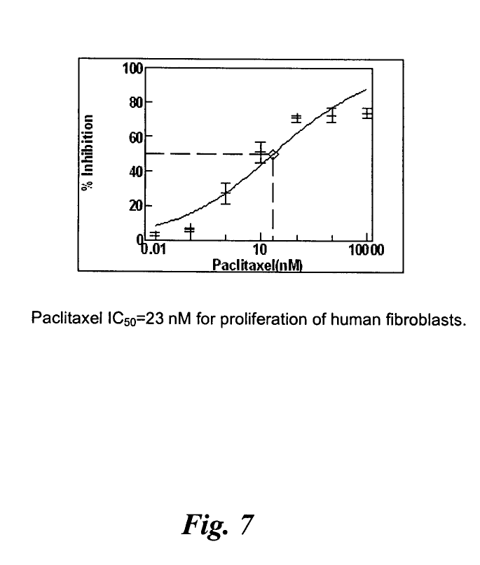

To terminate the assay, the media was removed by gentle aspiration. A 1/400 dilution of CYQUANT 400× GR dye indicator (Molecular Probes; Eugene, Oreg.) was added to 1× Cell Lysis buffer, and 200 μL of the mixture was added to the wells of the plate. Plates were incubated at room temperature, protected from light for 3-5 minutes. Fluorescence was read in a fluorescence microplate reader at ˜480 nm excitation wavelength and ˜520 nm emission maxima. Inhibitory concentration of 50% (IC₅₀) was determined by taking the average of triplicate wells and comparing average relative fluorescence units to the DMSO control. An average of n=4 replicate experiments was used to determine IC₅₀ values. The IC₅₀ values for the following compounds were determined using this assay: IC₅₀ (nM): mitoxantrone, 20 (FIG. 5); rapamycin, 19 (FIG. 6); paclitaxel, 23 (FIG. 7); mycophenolic acid, 550; mofetil, 601; GW8510, 98; simvastatin, 885; doxorubicin, 84; geldanamycin, 11; anisomycin, 435; 17-AAG, 106; bleomycin, 86; halofuginone, 36; gemfibrozil, 164; ciprofibrate, 503; bezafibrate, 184; epirubicin hydrochloride, 57; topotemay, 81; fascaplysin, 854; tamoxifen, 13; etanidazole, 55; gemcitabine, 7; puromycin, 254; mithramycin, 156; daunorubicin, 51; L(−)-perillyl alcohol, 966; celastrol, 271; anacitabine, 225; oxalipatin, 380; chromomycin A3, 4; vinorelbine, 4; idarubicin, 34; nogalamycin, 5; 17-DMAG, 5; epothilone D, 2; vinblastine, 2; vincristine, 7; cytarabine, 137.

### Example 25

### Evaluation of Paclitaxel Containing Mesh on Intimal Hyperplasia Development in a Rat Balloon Injury Cartoid Artery Model as as Example to Evaluate Fibrosis Inhibiting Agents

A rat balloon injury carotid artery model was used to demonstrate the efficacy of a paclitaxel containing mesh system on the development of intimal hyperplasia fourteen days following placement.

Control Group

Wistar rats weighing 400-500 g were anesthetized with 1.5% halothane in oxygen and the left external carotid artery was exposed. An A 2 French FOGARTY balloon embolectomy catheter (Baxter, Irvine, Calif.) was advanced through an arteriotomy in the external carotid artery down the left common carotid artery to the aorta. The balloon was inflated with enough saline to generate slight resistance (approximately 0.02 ml) and it was withdrawn with a twisting motion to the carotid bifurcation. The balloon was then deflated and the procedure repeated twice more. This technique produced distension of the arterial wall and denudation of the endothelium. The external carotid artery was ligated after removal of the catheter. The right common carotid artery was not injured and was used as a control.

Local Perivascular Paclitaxel Treatment

Immediately after injury of the left common carotid artery, a 1 cm long distal segment of the artery was exposed and treated with a 1×1 cm paclitaxel-containing mesh (345 ug paclitaxel in a 50:50 PLG coating on a 10:90 PLG mesh). The wound was then closed the animals were kept for 14 days.

Histology and immunohistochemistry

At the time of sacrifice, the animals were euthanized with carbon dioxide and pressure perfused at 100 mmHg with 10% phosphate buffered formaldehyde for 15 minutes. Both carotid arteries were harvested and left overnight in fixative. The fixed arteries were processed and embedded in paraffin wax. Serial cross-sections were cut at 3 μm thickness every 2 mm within and outside the implant region of the injured left carotid artery and at corresponding levels in the control right carotid artery. Cross-sections were stained with Mayer's hematoxylin-and-eosin for cell count and with Movat's pentachrome stains for morphometry analysis and for extracellular matrix composition assessment.

Results

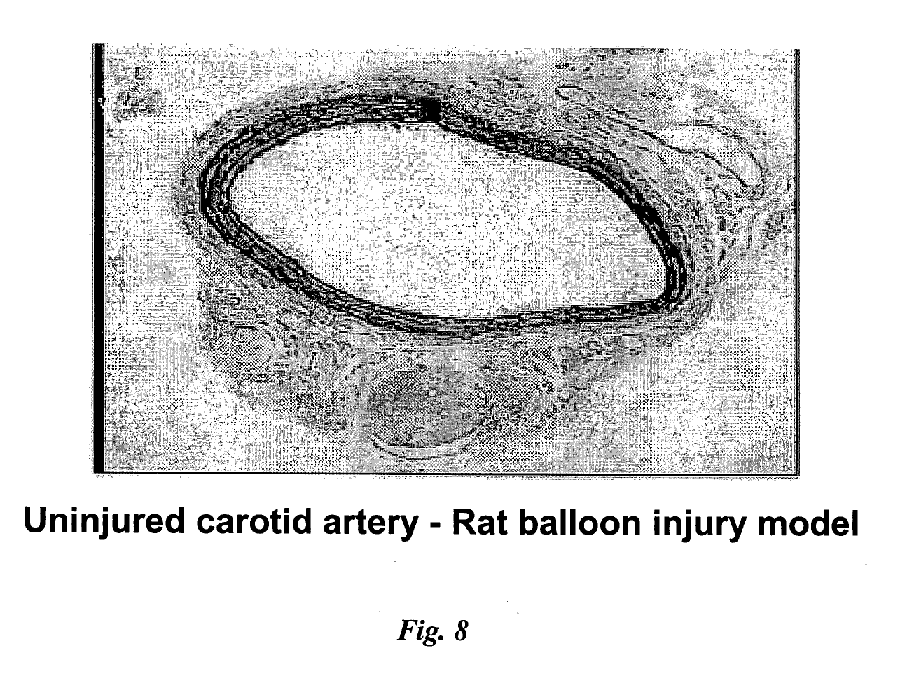

From FIGS. 8-10, it is evident that the perivascular delivery of paclitaxel using the paclitaxel mesh formulation resulted is a dramatic reduction in intimal hyperplasia.

### Example 26

### Effect of Paclitaxel and other Anti-Microtubule Agents on Matrix Metalloproteinase Production

A. Materials and Methods

1) IL-1 Stimulated AP-1 Transcriptional Activity is Inhibited by Paclitaxel

Chondrocytes were transfected with constructs containing an AP-1 driven CAT reporter gene, and stimulated with IL-1, IL-1 (50 ng/ml) was added and incubated for 24 hours in the absence and presence of paclitaxel at various concentrations. Paclitaxel treatment decreased CAT activity in a concentration dependent manner (mean ±SD). The data noted with an asterisk (*) have significance compared with IL-1-induced CAT activity according to a t-test, P<0.05. The results shown are representative of three independent experiments.

2) Effect of Paclitaxel on IL-1 Induced AP-1 DNA Binding Activity, AP-1 DNA

Binding activity was assayed with a radiolabeled human AP-1 sequence probe and gel mobility shift assay. Extracts from chondrocytes untreated or treated with various amounts of paclitaxel (10⁻⁷ to 10⁻⁵ M) followed by IL-1β (20 ng/ml) were incubated with excess probe on ice for 30 minutes, followed by non-denaturing gel electrophoresis. The “com” lane contains excess unlabeled AP-1 oligonucleotide. The results shown are representative of three independent experiments.

3) Effect of Paclitaxel on IL-1 Induced MMP-1 and MMP-3 mRNA Expression

Cells were treated with paclitaxel at various concentrations (10⁻⁷ to 10⁻⁵ M) for 24 hours, then treated with IL-1, (20 ng/ml) for additional 18 hours in the presence of paclitaxel. Total RNA was isolated, and the MMP-1 mRNA levels were determined by Northern blot analysis. The blots were subsequently stripped and reprobed with ³²P-radiolabeled rat GAPDH cDNA, which was used as a housekeeping gene. The results shown are representative of four independent experiments. Quantitation of collagenase-1 and stromelysin-expression mRNA levels were conducted. The MMP-1 -and MMP-3 expression levels were normalized with GAPDH.

4) Effect of other Anti-Microtubules on Collagenase Expression

Primary chondrocyte cultures were freshly isolated from calf cartilage. The cells were plated at 2.5×10⁶ per ml in 100×20 mm culture dishes and incubated in Ham's F12 medium containing 5% FBS overnight at 37° C. The cells were starved in serum-free medium overnight and then treated with anti-microtubule agents at various concentrations for 6 hours. IL-1 (20 ng/ml) was then added to each plate and the plates incubated for an additional 18 hours. Total RNA was isolated by the acidified guanidine isothiocyanate method and subjected to electrophoresis on a denatured gel. Denatured RNA samples (15 μg) were analyzed by gel electrophoresis in a 1% denatured gel, transferred to a nylon membrane and hydridized with the ³²P-labeled collagenase cDNA probe. 32P-labeled glyceraldehyde phosphate dehydrase (GAPDH) cDNA as an internal standard to ensure roughly equal loading. The exposed films were scanned and quantitatively analyzed with IMAGEQUANT.

B. Results

1) Promoters on the Family of Matrix Metalloproteinases

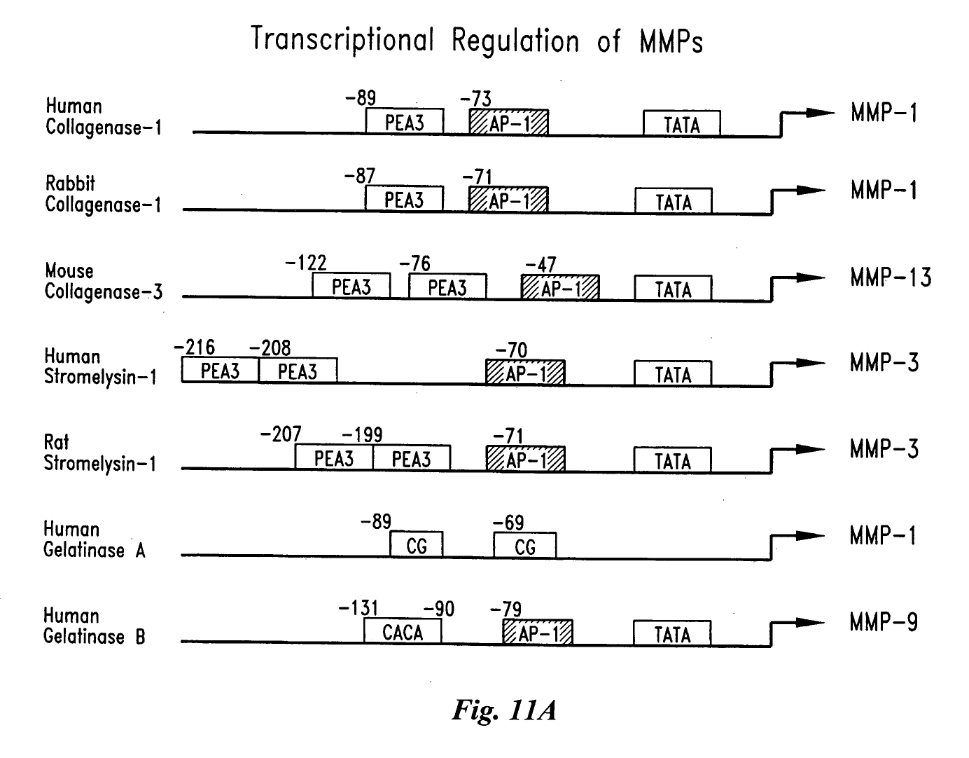

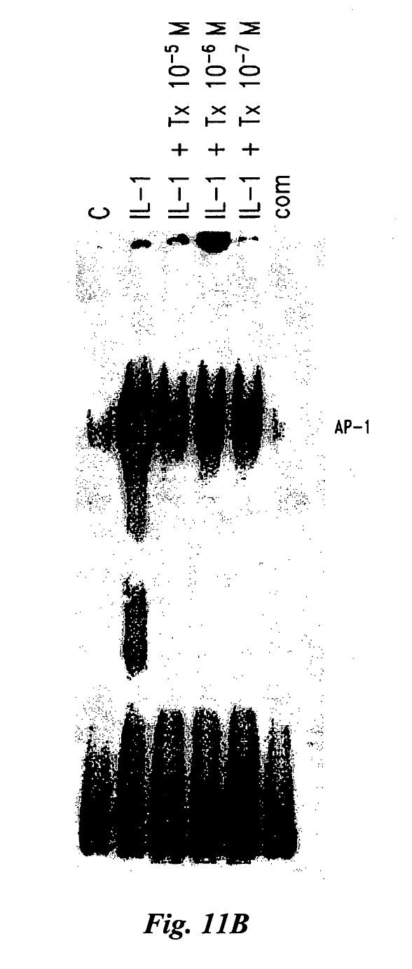

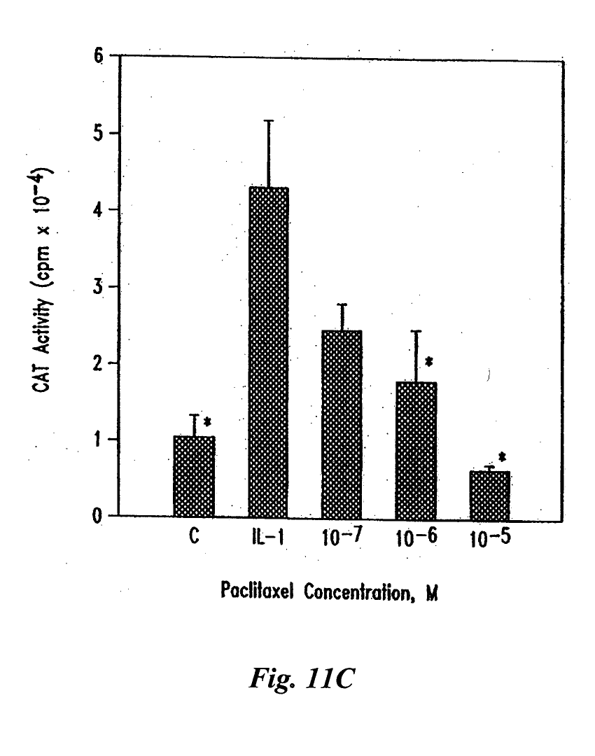

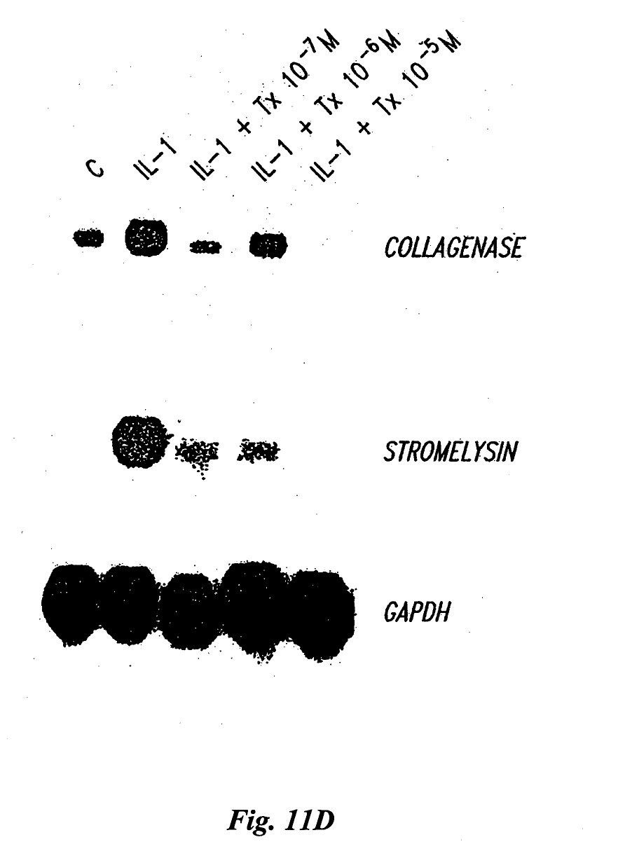

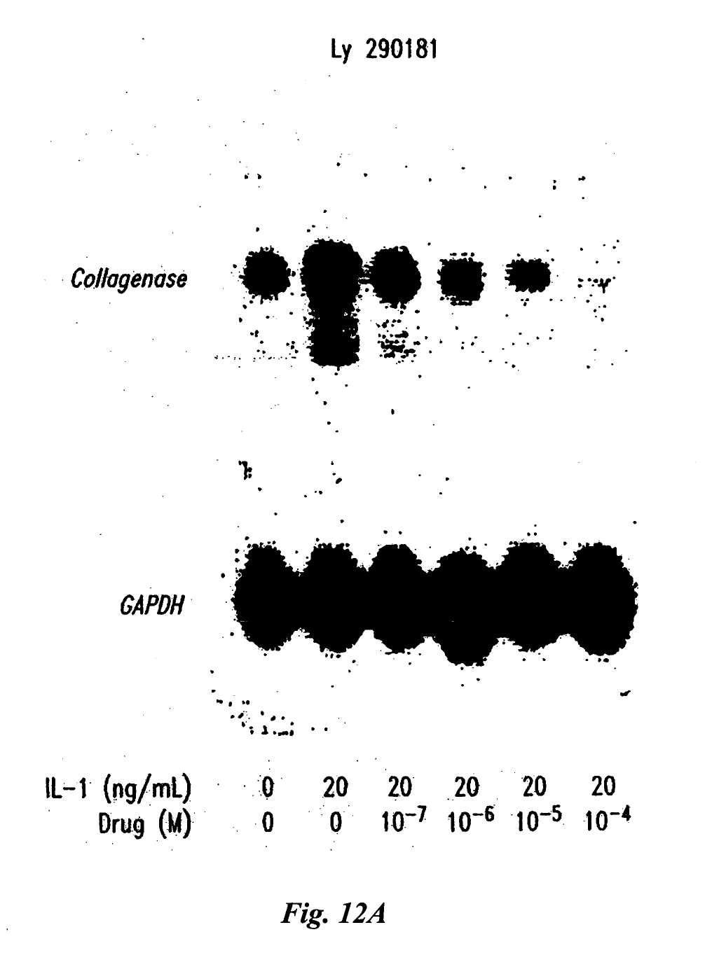

FIG. 11A shows that all matrix metalloproteinases contained the transcriptional elements AP-1 and PEA-3 with the exception of gelatinase B. It has been well established that expression of matrix metalloproteinases such as collagenases and stromelysins are dependent on the activation of the transcription factors AP-1. Thus inhibitors of AP-1 may inhibit the expression of matrix metalloproteinases.

2) Effect of Paclitaxel on AP-1 Transcriptional Activity

As demonstrated in FIG. 11B, IL-1 stimulated AP-1 transcriptional activity 5-fold. Pretreatment of transiently transfected chondrocytes with paclitaxel reduced IL-1 induced AP-1 reporter gene CAT activity. Thus, IL-1 induced AP-1 activity was reduced in chondrocytes by paclitaxel in a concentration dependent manner (10⁻⁷ to 10⁻⁵ M). These data demonstrated that paclitaxel was a potent inhibitor of AP-1 activity in chondrocytes.

3) Effect of Paclitaxel on AP-1 DNA Binding Activity

To confirm that paclitaxel inhibition of AP-1 activity was not due to nonspecific effects, the effect of paclitaxel on IL-1 induced AP-1 binding to oligonucleotides using chondrocyte nuclear lysates was examined. As shown in FIG. 11C, IL-1 induced binding activity decreased in lysates from chondrocyte which had been pretreated with paclitaxel at concentration 10⁻⁷ to 10⁻⁵ M for 24 hours. Paclitaxel inhibition of AP-1 transcriptional activity closely correlated with the decrease in AP-1 binding to DNA.

4) Effect of Paclitaxel on Collagenase and Stromelysin Expression

Since paclitaxel was a potent inhibitor of AP-1 activity, the effect of paclitaxel or IL-1 induced collagenase and stromelysin expression, two important matrix metalloproteinases involved in inflammatory diseases was examined. Briefly, as shown in FIG. 11 D, IL-1 induction increases collagenase and stromelysin mRNA levels in chondrocytes. Pretreatment of chondrocytes with paclitaxel for 24 hours significantly reduced the levels of collagenase and stromelysin mRNA. At 10⁻⁵ M paclitaxel, there was complete inhibition. The results show that paclitaxel completely inhibited the expression of two matrix metalloproteinases at concentrations similar to which it inhibits AP-1 activity.

5) Effect of other Anti-Microtubules on Collagenase Expression

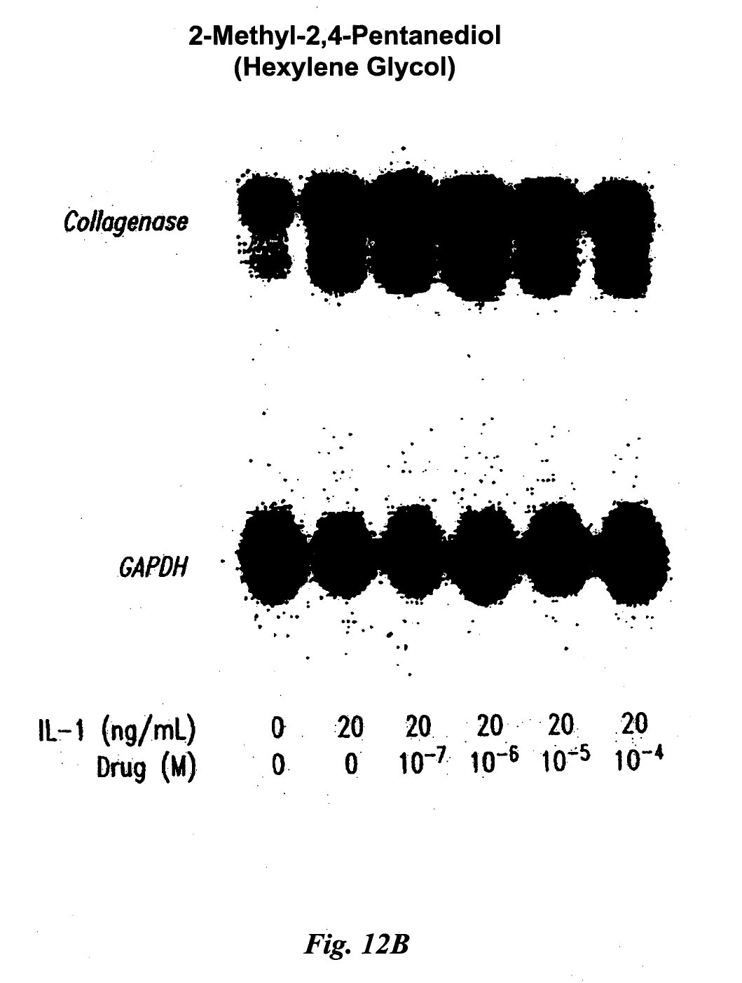

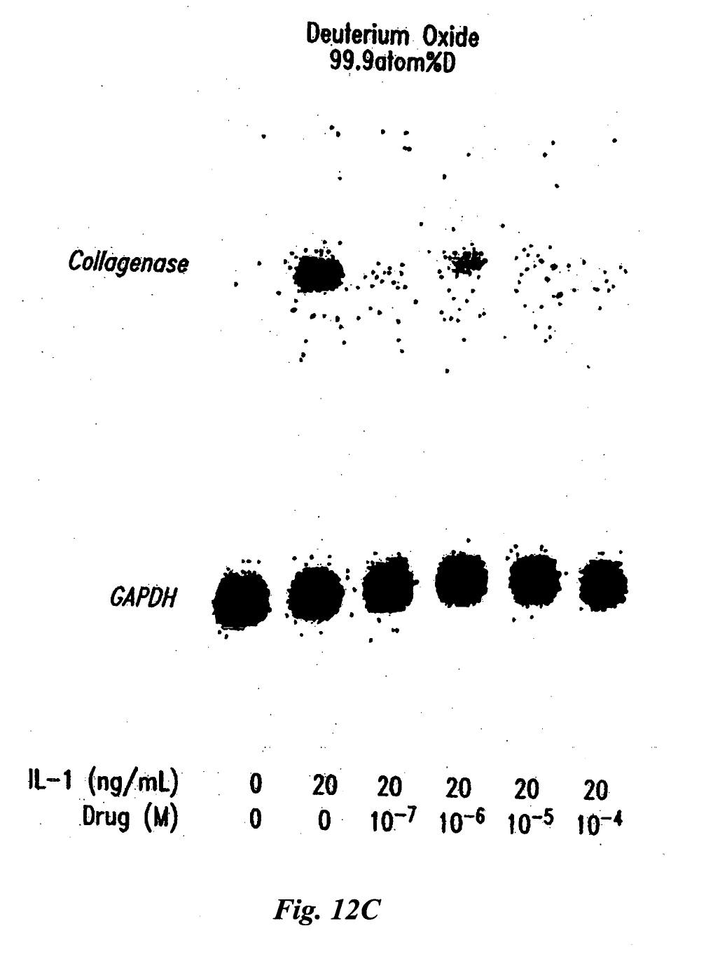

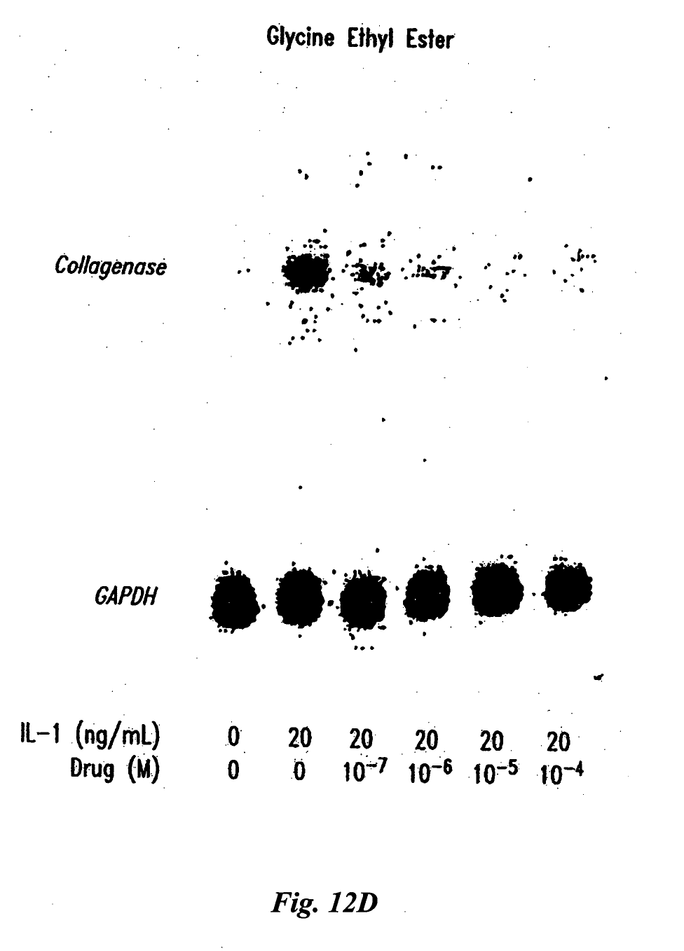

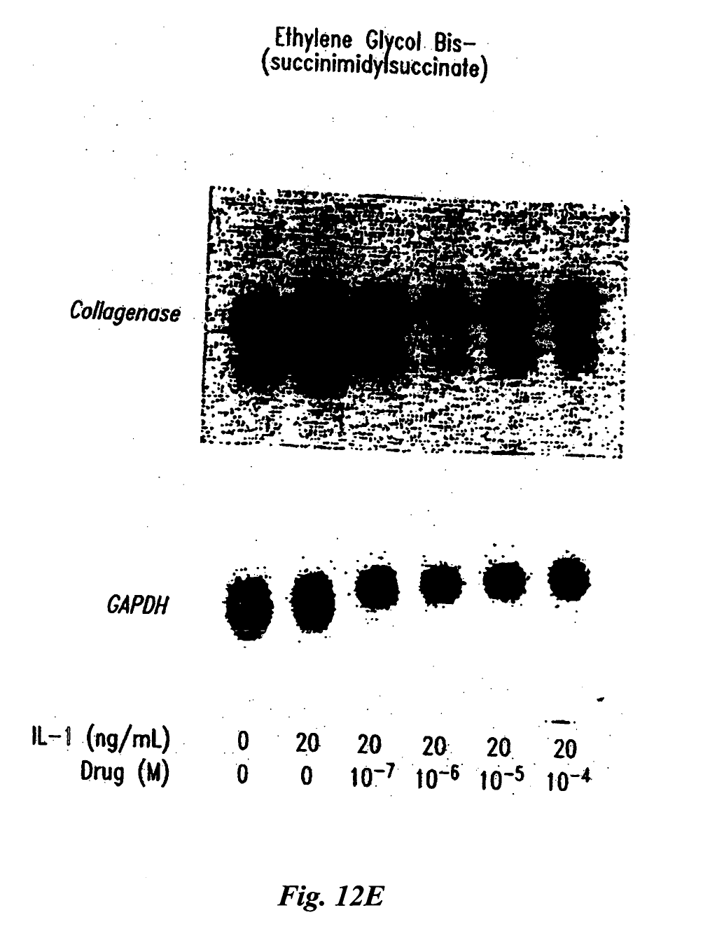

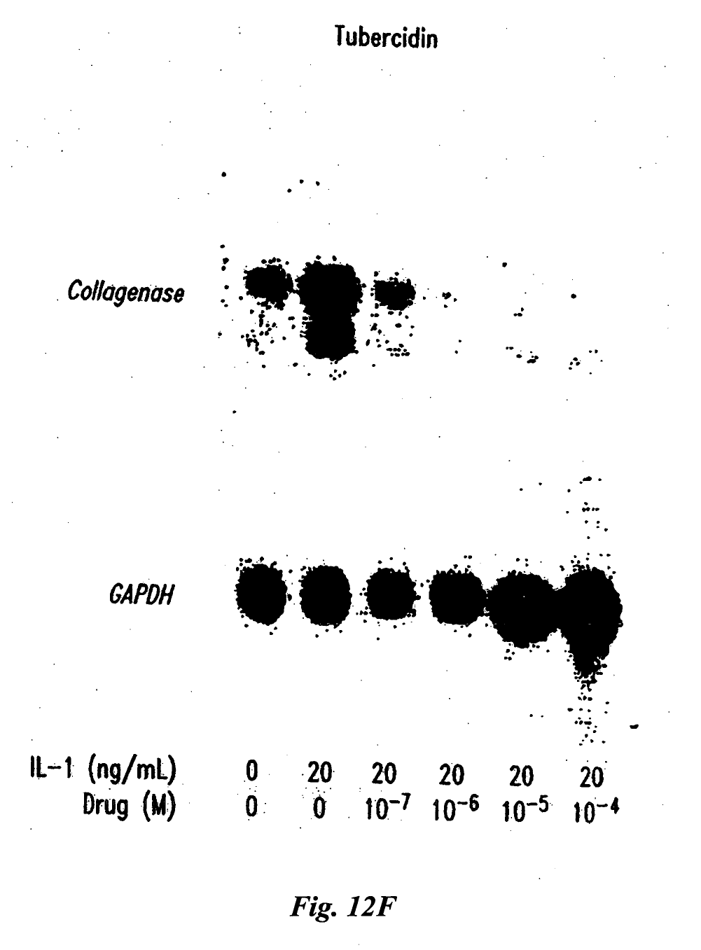

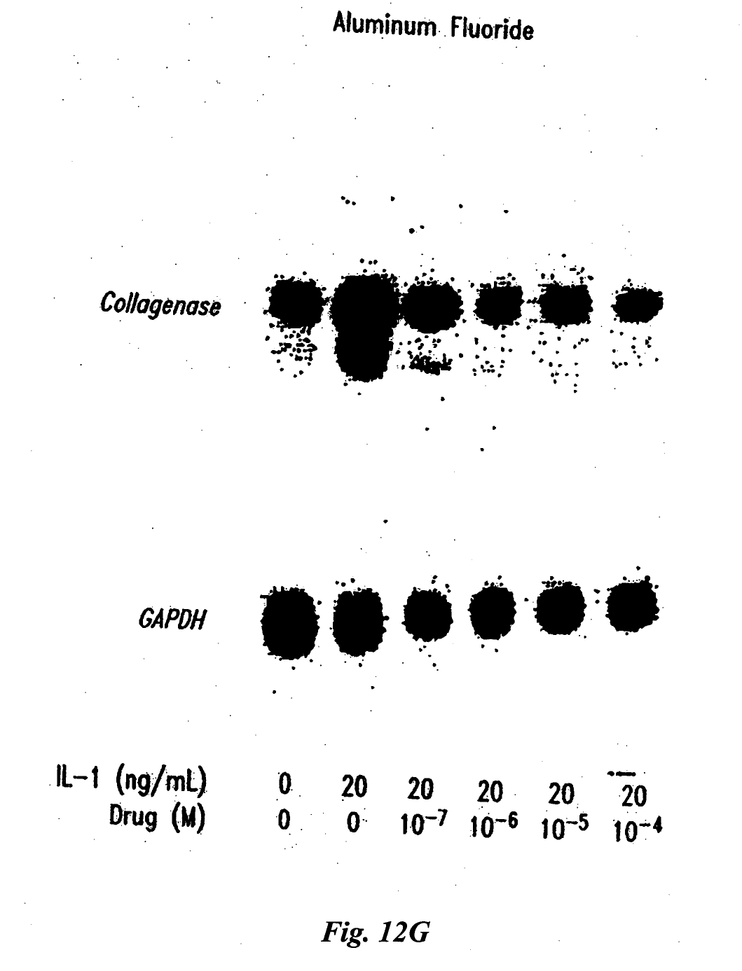

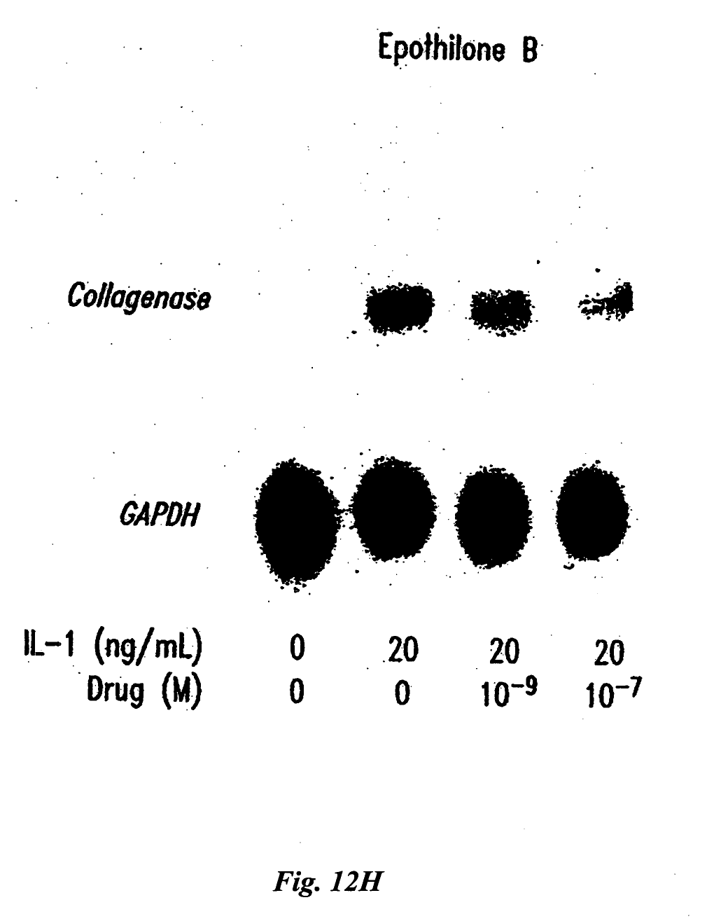

FIGS. 12A-H demonstrate that anti-microtubule agents inhibited collagenase expression. Expression of collagenase was stimulated by the addition of IL-1 which is a proinflammatory cytokine. Pre-incubation of chondrocytes with various anti-microtubule agents, specifically LY290181, hexylene glycol, deuterium oxide, glycine ethyl ester, ethylene glycol bis-(succinimidylsuccinate), tubercidin, AlF₃, and epothilone, all prevented IL-1-induced collagenase expression at concentrations as low as 1×10⁻⁷ M.

C. Discussion

Paclitaxel was capable of inhibiting collagenase and stromelysin expression in vitro at concentrations of 10⁻⁶ M. Since this inhibition may be explained by the inhibition of AP-1 activity, a required step in the induction of all matrix metalloproteinases with the exception of gelatinase B, it is expected that paclitaxel may inhibit other matrix metalloproteinases which are AP-1 dependent. The levels of these matrix metalloproteinases are elevated in all inflammatory diseases and play a principle role in matrix degradation, cellular migration and proliferation, and angiogenesis. Thus, paclitaxel inhibition of expression of matrix metalloproteinases such as collagenase and stromelysin can have a beneficial effect in inflammatory diseases.

In addition to paclitaxel's inhibitory effect on collagenase expression, LY290181, hexylene glycol, deuterium oxide, glycine ethyl ester, AIF₃, tubercidin epothilone, and ethylene glycol bis-(succinimidylsuccinate), all prevented IL-1-induced collagenase expression at concentrations as low as 1×10⁻⁷ M. Thus, anti-microtubule agents are capable of inhibiting the AP-1 pathway at varying concentrations.

### Example 27

### Inhibition of Angiogenisis by Paclitaxel

A. Chick Chorioallantoic Membrane (“CAM”) Assays

Fertilized, domestic chick embryos were incubated for 3 days prior to shell-less culturing. In this procedure, the egg contents were emptied by removing the shell located around the air space. The interior shell membrane was then severed and the opposite end of the shell was perforated to allow the contents of the egg to gently slide out from the blunted end. The egg contents were emptied into round-bottom sterilized glass bowls and covered with petri dish covers. These were then placed into an incubator at 90% relative humidity and 3% CO₂ and incubated for 3 days.

Paclitaxel (Sigma, St. Louis, Mich.) was mixed at concentrations of 0.25, 0.5, 1, 5, 10, 30 μg per 10 ul aliquot of 0.5% aqueous methylcellulose. Since paclitaxel is insoluble in water, glass beads were used to produce fine particles. Ten microliter aliquots of this solution were dried on parafilm for 1 hour forming disks 2 mm in diameter. The dried disks containing paclitaxel were then carefully placed at the growing edge of each CAM at day 6 of incubation. Controls were obtained by placing paclitaxel-free methylcellulose disks on the CAMs over the same time course. After a 2 day exposure (day 8 of incubation) the vasculature was examined with the aid of a stereomicroscope. Liposyn II, a white opaque solution, was injected into the CAM to increase the visibility of the vascular details. The vasculature of unstained, living embryos were imaged using a Zeiss stereomicroscope which was interfaced with a video camera (Dage-MTI Inc., Michigan City, Ind.). These video signals were then displayed at 160× magnification and captured using an image analysis system (Vidas, Kontron; Etching, Germany). Image negatives were then made on a graphics recorder (Model 3000; Matrix Instruments, Orangeburg, N.Y.).

The membranes of the 8 day-old shell-less embryo were flooded with 2% glutaraldehyde in 0.1M sodium cacodylate buffer; additional fixative was injected under the CAM. After 10 minutes in situ, the CAM was removed and placed into fresh fixative for 2 hours at room temperature. The tissue was then washed overnight in cacodylate buffer containing 6% sucrose. The areas of interest were postfixed in 1% osmium tetroxide for 1.5 hours at 4° C. The tissues were then dehydrated in a graded series of ethanols, solvent exchanged with propylene oxide, and embedded in Spurr resin. Thin sections were cut with a diamond knife, placed on copper grids, stained, and examined in a Joel 1200EX electron microscope. Similarly, 0.5 mm sections were cut and stained with toluene blue for light microscopy.

At day 11 of development, chick embryos were used for the corrosion casting technique. Mercox resin (Ted Pella, Inc., Redding, Calif.) was injected into the CAM vasculature using a 30-gauge hypodermic needle. The casting material consisted of 2.5 grams of Mercox CL-2B polymer and 0.05 grams of catalyst (55% benzoyl peroxide) having a 5 minute polymerization time. After injection, the plastic was allowed to sit in situ for an hour at room temperature and then overnight in an oven at 65° C. The CAM was then placed in 50% aqueous solution of sodium hydroxide to digest all organic components. The plastic casts were washed extensively in distilled water, air-dried, coated with gold/palladium, and viewed with the Philips 501 B scanning electron microscope.

Results of the assay were as follows. At day 6 of incubation, the embryo was centrally positioned to a radially expanding network of blood vessels; the CAM developed adjacent to the embryo. These growing vessels lie close to the surface and are readily visible making this system an idealized model for the study of angiogenesis. Living, unstained capillary networks of the CAM may be imaged noninvasively with a stereomicroscope.

Transverse sections through the CAM show an outer ectoderm consisting of a double cell layer, a broader mesodermal layer containing capillaries which lie subjacent to the ectoderm, adventitial cells, and an inner, single endodermal cell layer. At the electron microscopic level, the typical structural details of the CAM capillaries are demonstrated. Typically, these vessels lie in close association with the inner cell layer of ectoderm.

After 48 hours exposure to paclitaxel at concentrations of 0.25, 0.5, 1, 5, 10, or 30 μg, each CAM was examined under living conditions with a stereomicroscope equipped with a video/computer interface in order to evaluate the effects on angiogenesis. This imaging setup was used at a magnification of 160× which permitted the direct visualization of blood cells within the capillaries; thereby blood flow in areas of interest may be easily assessed and recorded. For this study, the inhibition of angiogenesis was defined as an area of the CAM (measuring 2-6 mm in diameter) lacking a capillary network and vascular blood flow. Throughout the experiments, avascular zones were assessed on a 4 point avascular gradient (Table 1). This scale represents the degree of overall inhibition with maximal inhibition represented as a 3 on the avascular gradient scale. Paclitaxel was very consistent and induced a maximal avascular zone (6 mm in diameter or a 3 on the avascular gradient scale) within 48 hours depending on its concentration.

TABLE 1
Avascular Gradient

| | |
|---|---|
| 0 | normal vascularity |
| 1 | lacking some microvascular movement |
| 2* | small avascular zone approximately 2 mm in diameter |
| 3* | avascularity extending beyond the disk (6 mm in diameter) |

*indicates a positive antiangiogenesis response

The dose-dependent, experimental data of the effects of paclitaxel at different concentrations are shown in Table 2.

TABLE 2

| Agent | Delivery Vehicle | Concentration | Inhibition/n |
|---|---|---|---|
| paclitaxel | methylcellulose (10 ul) | 0.25 ug | 2/11 |
| | methylcellulose (10 ul) | 0.5 ug | 6/11 |
| | methylcellulose (10 ul) | 1 ug | 6/15 |
| | methylcellulose (10 ul) | 5 ug | 20/27 |
| | methylcellulose (10 ul) | 10 ug | 16/21 |
| | methylcellulose (10 ul) | 30 ug | 31/31 |

Typical paclitaxel-treated CAMs are also shown with the transparent methylcellulose disk centrally positioned over the avascular zone measuring 6 mm in diameter. At a slightly higher magnification, the periphery of such avascular zones is clearly evident; the surrounding functional vessels were often redirected away from the source of paclitaxel. Such angular redirecting of blood flow was never observed under normal conditions. Another feature of the effects of paclitaxel was the formation of blood islands within the avascular zone representing the aggregation of blood cells.

In summary, this study demonstrated that 48 hours after paclitaxel application to the CAM, angiogenesis was inhibited. The blood vessel inhibition formed an avascular zone which was represented by three transitional phases of paclitaxel's effect. The central, most affected area of the avascular zone contained disrupted capillaries with extravasated red blood cells; this indicated that intercellular junctions between endothelial cells were absent. The cells of the endoderm and ectoderm maintained their intercellular junctions and therefore these germ layers remained intact; however, they were slightly thickened. As the normal vascular area was approached, the blood vessels retained their junctional complexes and therefore also remained intact. At the periphery of the paclitaxel-treated zone, further blood vessel growth was inhibited which was evident by the typical redirecting or “elbowing” effect of the blood vessels.

### Example 28

### Screening Assay for Assessing the Effect of Paclitaxel on Smooth Muscle Cell Migration

Primary human smooth muscle cells were starved of serum in smooth muscle cell basal media containing insulin and human basic fibroblast growth factor (bFGF) for 16 hours prior to the assay. For the migration assay, cells were trypsinized to remove cells from flasks, washed with migration media and diluted to a concentration of 2-2.5×10⁵ cells/ml in migration media. Migration media consists of phenol red free Dulbecco's Modified Eagle Medium (DMEM) containing 0.35% human serum albumin. A 100 μL volume of smooth muscle cells (approximately 20,000-25,000 cells) was added to the top of a Boyden chamber assembly (Chemicon QCM CHEMOTAXIS 96-well migration plate). To the bottom wells, the chemotactic agent, recombinant human platelet derived growth factor (rhPDGF-BB) was added at a concentration of 10 ng/ml in a total volume of 150 μL. Paclitaxel was prepared in DMSO at a concentration of 10⁻² M and serially diluted 10-fold to give a range of stock concentrations (10⁻⁸ M to 10⁻² M). Paclitaxel was added to cells by directly adding paclitaxel DMSO stock solutions, prepared earlier, at a 1/1000 dilution, to the cells in the top chamber. Plates were incubated for 4 hours to allow cell migration.

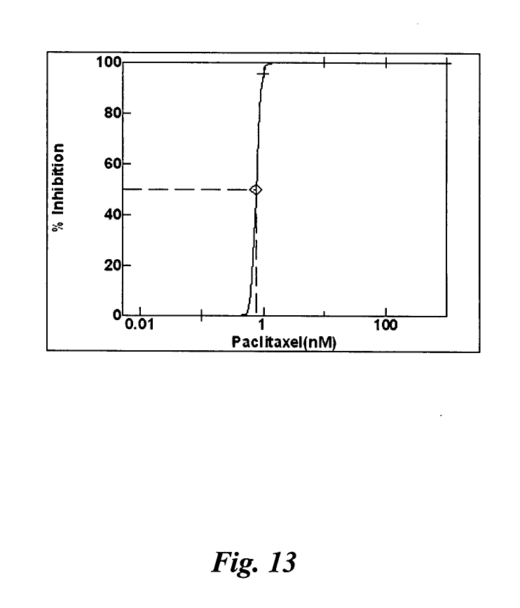

At the end of the 4 hour period, cells in the top chamber were discarded and the smooth muscle cells attached to the underside of the filter were detached for 30 minutes at 37° C. in Cell Detachment Solution (Chemicon). Dislodged cells were lysed in lysis buffer containing the DNA binding CYQUANT GR dye and incubated at room temperature for 15 minutes. Fluorescence was read in a fluorescence microplate reader at ˜480 nm excitation wavelength and ˜520 nm emission maxima. Relative fluorescence units from triplicate wells were averaged after subtracting background fluorescence (control chamber without chemoattractant) and average number of cells migrating was obtained from a standard curve of smooth muscle cells serially diluted from 25,000 cells/well down to 98 cells/well. Inhibitory concentration of 50% (IC₅₀) was determined by comparing the average number of cells migrating in the presence of paclitaxel to the positive control (smooth muscle cell chemotaxis in response to rhPDGF-BB). See FIG. 13 ( IC₅₀=0.76 nM). References: Biotechniques (2000) 29: 81; J. Immunol Methods (2001) 254: 85

### Example 29

### Screening Assay for Assessing the Effect of Various Compounds on IL-1β Production by Macrophages

The human macrophage cell line, THP-1 was plated in a 12 well plate such that each well contains 1×10⁶ cells in 2 ml of media containing 10% FCS. Opsonized zymosan was prepared by resuspending 20 mg of zymosan A in 2 ml of ddH₂O and homogenizing until a uniform suspension was obtained. Homogenized zymosan was pelleted at 250 g and resuspended in 4 ml of human serum for a final concentration of 5 mg/ml and incubated in a 37° C. water bath for 20 minutes to enable opsonization. Geldanamycin was prepared in DMSO at a concentration of 10⁻² M and serially diluted 10-fold to give a range of stock concentrations (10⁻⁸ M to 10⁻² M).

THP-1 cells were stimulated to produce IL-1, by the addition of 1 mg/ml opsonized zymosan. Geldanamycin was added to THP-1 cells by directly adding DMSO stock solutions, prepared earlier, at a 1/1000 dilution, to each well. Each drug concentration was tested in triplicate wells. Plates were incubated at 37° C. for 24 hours.

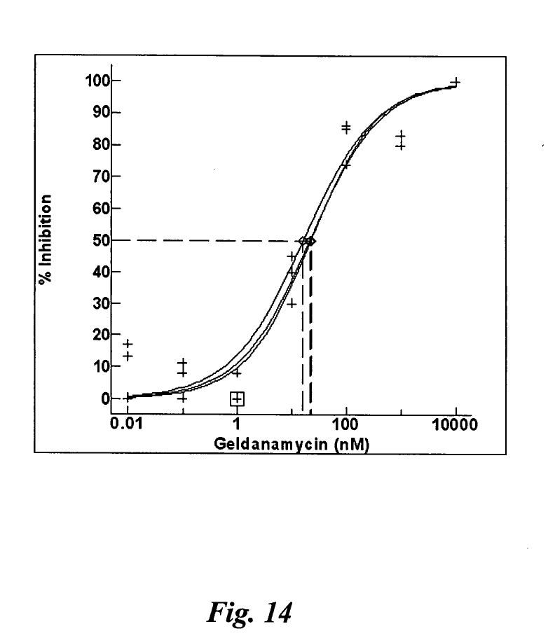

After a 24 hour stimulation, supernatants were collected to quantify IL-1β production. IL-1β concentrations in the supernatants were determined by ELISA using recombinant human IL-1β to obtain a standard curve. A 96-well MaxiSorb plate was coated with 100 μL of anti-human IL-1β Capture Antibody diluted in Coating Buffer (0.1 M Sodium carbonate pH 9.5) overnight at 4° C. The dilution of Capture Antibody used was lot-specific and was determined empirically. Capture antibody was then aspirated and the plate washed 3 times with Wash Buffer (PBS, 0.05% TWEEN-20). Plates were blocked for 1 hour at room temperature with 200 μL/well of Assay Diluent (PBS, 10% FCS pH 7.0). After blocking, plates were washed 3 times with Wash Buffer. Standards and sample dilutions were prepared as follows: (a) sample supernatants were diluted ¼ and ⅛; (b) recombinant human IL-1β was prepared at 1000 pg/ml and serially diluted to yield as standard curve of 15.6 pg/ml to 1000 pg/ml. Sample supernatants and standards were assayed in triplicate and were incubated at room temperature for 2 hours after addition to the plate coated with Capture Antibody. The plates were washed 5 times and incubated with 100 μL of Working Detector (biotinylated anti-human IL-1β detection antibody+avidin-HRP) for 1 hour at room temperature. Following this incubation, the plates were washed 7 times and 100 μL of Substrate Solution (Tetramethylbenzidine, H₂O₂) was added to plates and incubated for 30 minutes at room temperature. Stop Solution (2 N H₂SO₄) was then added to the wells and a yellow color reaction was read at 450 nm with A correction at 570 nm. Mean absorbance was determined from triplicate data readings and the mean background was subtracted. IL-1β concentration values were obtained from the standard curve. Inhibitory concentration of 50% (IC₅₀) was determined by comparing average IL-1β concentration to the positive control (THP-1 cells stimulated with opsonized zymosan). An average of n=4 replicate experiments was used to determine IC₅₀ values for geldanamycin (IC₅₀=20 nM). See FIG. 14. The IC₅₀ values for the following additional compounds were determined using this assay: IC₅₀ (nM): mycophenolic acid 2888 nM); anisomycin, 127; rapamycin, 0.48; halofuginone, 919; IDN-6556, 642; epirubicin hydrochloride, 774; topotemay, 509; fascaplycin, 425; daunorubicin, 517; celastrol, 23; oxalipatin, 107; chromomycin A3, 148.

References: J. Immunol. (2000) 165: 411-418; J. Immunol. (2000) 164: 4804-4811; J. Immunol Meth. (2000) 235 (1-2): 33-40.

### Example 30

### Screening Assay for Assessing the Effect of Various Compounds on IL-8 Production by Macrophages

The human macrophage cell line, THP-1 was plated in a 12 well plate such that each well contains 1×10⁶ cells in 2 ml of media containing 10% FCS. Opsonized zymosan was prepared by resuspending 20 mg of zymosan A in 2 ml of ddH₂O and homogenizing until a uniform suspension was obtained. Homogenized zymosan was pelleted at 250 g, resuspended in 4 ml of human serum for a final concentration of 5 mg/ml, and incubated in a 37° C. water bath for 20 minutes to enable opsonization. Geldanamycin was prepared in DMSO at a concentration of 10⁻² M and serially diluted 10-fold to give a range of stock concentrations (10⁻⁸ M to 10⁻² M).

THP-1 cells were stimulated to produce IL-8 by the addition of 1 mg/ml opsonized zymosan. Geldanamycin was added to THP-1 cells by directly adding DMSO stock solutions, prepared earlier, at a 1/1000 dilution, to each well. Each drug concentration was tested in triplicate wells. Plates were incubated at 37° C. for 24 hours.

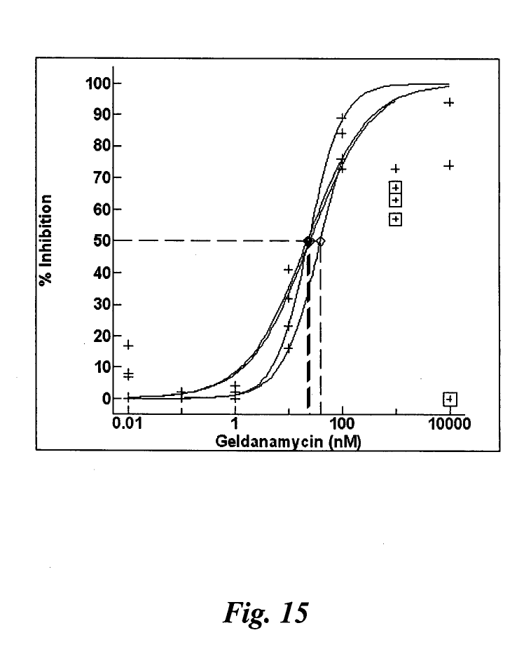

After a 24 hour stimulation, supernatants were collected to quantify IL-8 production. IL-8 concentrations in the supernatants were determined by ELISA using recombinant human IL-8 to obtain a standard curve. A 96-well MAXISORB plate was coated with 100 μL of anti-human IL-8 Capture Antibody diluted in Coating Buffer (0.1 M sodium carbonate pH 9.5) overnight at 4° C. The dilution of Capture Antibody used was lot-specific and was determined empirically. Capture antibody was then aspirated and the plate washed 3 times with Wash Buffer (PBS, 0.05% TWEEN-20). Plates were blocked for 1 hour at room temperature with 200 μL/well of Assay Diluent (PBS, 10% FCS pH 7.0). After blocking, plates were washed 3 times with Wash Buffer. Standards and sample dilutions were prepared as follows: (a) sample supernatants were diluted 1/10o and 1/1000; (b) recombinant human IL-8 was prepared at 200 pg/ml and serially diluted to yield as standard curve of 3.1 pg/ml to 200 pg/ml. Sample supernatants and standards were assayed in triplicate and were incubated at room temperature for 2 hours after addition to the plate coated with Capture Antibody. The plates were washed 5 times and incubated with 100 μL of Working Detector (biotinylated anti-human IL-8 detection antibody 30 avidin-HRP) for 1 hour at room temperature. Following this incubation, the plates were washed 7 times and 100 μL of Substrate Solution (Tetramethylbenzidine, H₂O₂) was added to plates and incubated for 30 minutes at room temperature. Stop Solution (2 N H₂SO₄) was then added to the wells and a yellow color reaction was read at 450 nm with A correction at 570 nm. Mean absorbance was determined from triplicate data readings and the mean background was subtracted. IL-8 concentration values were obtained from the standard curve. Inhibitory concentration of 50% (IC₅₀) was determined by comparing average IL-8 concentration to the positive control (THP-1 cells stimulated with opsonized zymosan). An average of n=4 replicate experiments was used to determine IC₅₀ values for geldanamycin (IC₅₀=27 nM). See FIG. 15. The IC₅₀ values for the following additional compounds were determined using this assay: IC₅₀ (nM): 17-AAG, 56; mycophenolic acid, 549; resveratrol, 507; rapamycin, 4; 41; SP600125, 344; halofuginone, 641; D-mannose-6-phosphate, 220; epirubicin hydrochloride, 654; topotemay, 257; mithramycin, 33; daunorubicin, 421; celastrol, 490; chromomycin A3, 36.

References: J. Immunol. (2000) 165: 411-418; J. Immunol. (2000) 164: 4804-4811; J. Immunol Meth. (2000) 235 (1-2): 33-40.

### Example 31

### Screening Assay for Assessing the Effect of Various Compounds on MCP-1 Production by Macrophages

The human macrophage cell line, THP-1 was plated in a 12 well plate such that each well contains 1×10⁶ cells in 2 ml of media containing 10% FCS. Opsonized zymosan was prepared by resuspending 20 mg of zymosan A in 2 ml of ddH₂O and homogenizing until a uniform suspension was obtained. Homogenized zymosan was pelleted at 250 g and resuspended in 4 ml of human serum for a final concentration of 5 mg/ml and incubated in a 37° C. water bath for 20 minutes to enable opsonization. Geldanamycin was prepared in DMSO at a concentration of 10⁻² M and serially diluted 10-fold to give a range of stock concentrations (10⁻⁸ M to 10⁻² M).

THP-1 cells were stimulated to produce MCP-1 by the addition of 1 mg/ml opsonized zymosan. Eldanamycin was added to THP-1 cells by directly adding DMSO stock solutions, prepared earlier, at a 1/1000 dilution, to each well. Each drug concentration was tested in triplicate wells. Plates were incubated at 37° C. for 24 hours.

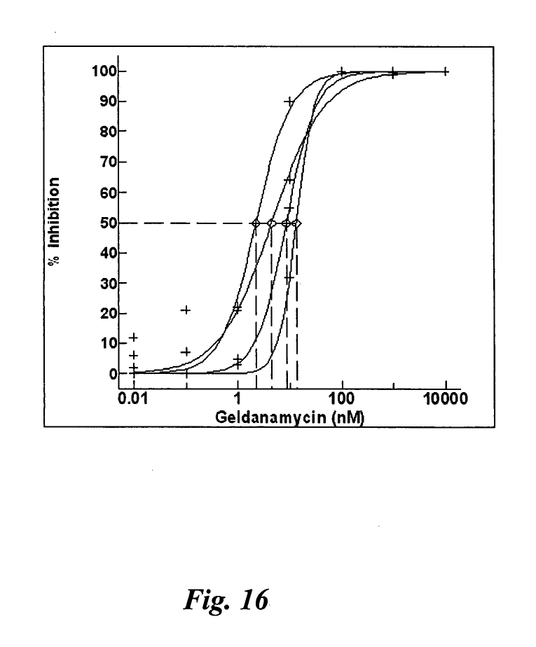

After a 24 hour stimulation, supernatants were collected to quantify MCP-1 production. MCP-1 concentrations in the supernatants were determined by ELISA using recombinant human MCP-1 to obtain a standard curve. A 96-well MaxiSorb plate was coated with 100 μL of anti-human MCP-1 Capture Antibody diluted in Coating Buffer (0.1 M sodium carbonate pH 9.5) overnight at 4° C. The dilution of Capture Antibody used was lot-specific and was determined empirically. Capture antibody was then aspirated and the plate washed 3 times with Wash Buffer (PBS, 0.05% TWEEN-20). Plates were blocked for 1 hour at room temperature with 200 μL/well of Assay Diluent (PBS, 10% FCS pH 7.0). After blocking, plates were washed 3 times with Wash Buffer. Standards and sample dilutions were prepared as follows: (a) sample supernatants were diluted 1/100 and 1/1000; (b) recombinant human MCP-1 was prepared at 500 pg/ml and serially diluted to yield as standard curve of 7.8 pg/ml to 500 pg/ml. Sample supernatants and standards were assayed in triplicate and were incubated at room temperature for 2 hours after addition to the plate coated with Capture Antibody. The plates were washed 5 times and incubated with 100 μL of Working Detector (biotinylated anti-human MCP-1 detection antibody+avidin-HRP) for 1 hour at room temperature. Following this incubation, the plates were washed 7 times and 100 μL of Substrate Solution (tetramethylbenzidine, H₂0₂) was added to plates and incubated for 30 minutes at room temperature. Stop Solution (2 N H₂SO₄) was then added to the wells and a yellow color reaction was read at 450 nm with A correction at 570 nm. Mean absorbance was determined from triplicate data readings and the mean background was subtracted. MCP-1 concentration values were obtained from the standard curve. Inhibitory concentration of 50% (IC₅₀) was determined by comparing average MCP-1 concentration to the positive control (THP-1 cells stimulated with opsonized zymosan). An average of n=4 replicate experiments was used to determine IC₅₀ values for geldanamycin (IC₅₀=7 nM). See FIG. 16. The IC₅₀ values for the following additional compounds were determined using this assay: IC₅₀ (nM): 17-AAG, 135; anisomycin, 71; mycophenolic acid, 764; mofetil, 217; mitoxantrone, 62; chlorpromazine, 0.011; 1-α-25 dihydroxy vitamin D₃, 1; Bay 58-2667, 216; 15-deoxy prostaglandin J2, 724; rapamycin, 0.05; CNI-1493, 0.02; BXT-51072, 683; halofuginone, 9; CYC 202, 306; topotemay, 514; fascaplycin, 215; podophyllotoxin, 28; gemcitabine, 50; puromycin, 161; mithramycin, 18; daunorubicin, 570; celastrol, 421; chromomycin A3, 37; vinorelbine, 69; tubercidin, 56; vinblastine, 19; vincristine, 16.

References: J. Immunol. (2000) 165: 411-418; J. Immunol. (2000) 164: 4804-4811; J. Immunol Meth. (2000) 235 (1-2): 33-40.

### Example 32

### Screening Assay for Assessing the Effect of Paclitaxel on Cell Proliferation

Smooth muscle cells at 70-90% confluency were trypsinized, replated at 600 cells/well in media in 96-well plates and allowed to attachment overnight. Paclitaxel was prepared in DMSO at a concentration of 10⁻² M and diluted 10-fold to give a range of stock concentrations (10⁻⁸ M to 10⁻² M). Drug dilutions were diluted 1/1000 in media and added to cells to give a total volume of 200 μL/well. Each drug concentration was tested in triplicate wells. Plates containing cells and paclitaxel were incubated at 37° C. for 72 hours.

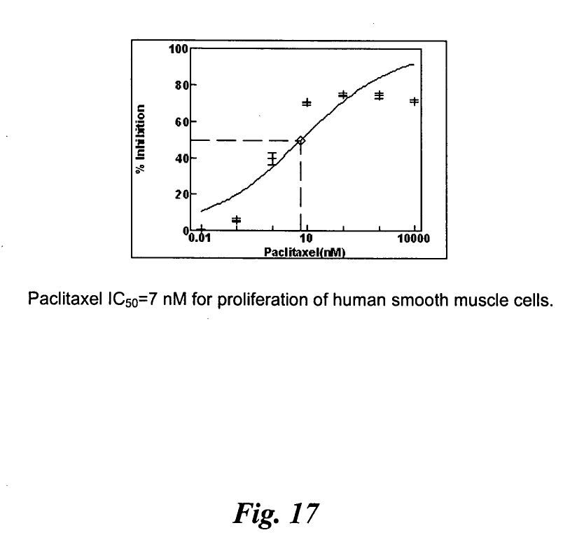

To terminate the assay, the media was removed by gentle aspiration. A 1/400 dilution of CYQUANT 400× GR dye indicator (Molecular Probes; Eugene, Oreg.) was added to 1× Cell Lysis buffer, and 200 μL of the mixture was added to the wells of the plate. Plates were incubated at room temperature, protected from light for 3-5 minutes. Fluorescence was read in a fluorescence microplate reader at -480 nm excitation wavelength and ˜520 nm emission maxima. Inhibitory concentration of 50% (IC₅₀) was determined by taking the average of triplicate wells and comparing average relative fluorescence units to the DMSO control. An average of n=3 replicate experiments was used to determine IC₅₀ values. See FIG. 17 (IC₅₀=7 nM). The IC₅₀ values for the following additional compounds were determined using this assay: IC₅₀ (nM): mycophenolic acid, 579; mofetil, 463; doxorubicin, 64; mitoxantrone, 1; geldanamycin, 5; anisomycin, 276; 17-AAG, 47; cytarabine, 85; halofuginone, 81; mitomycin C, 53; etoposide, 320; cladribine, 137; lovastatin, 978; epirubicin hydrochloride, 19; topotemay, 51; fascaplysin, 510; podophyllotoxin, 21; cytochalasin A, 221; gemcitabine, 9; puromycin, 384; mithramycin, 19; daunorubicin, 50; celastrol, 493; chromomycin A3, 12; vinorelbine, 15; idarubicin, 38; nogalamycin, 49; itraconazole, 795; 17-DMAG, 17; epothilone D, 5; tubercidin, 30; vinblastine, 3; vincristine, 9.

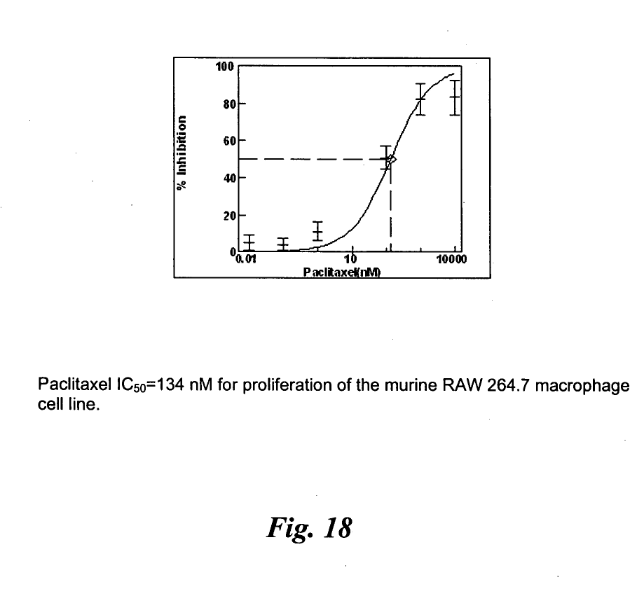

This assay also may be used assess the effect of compounds on proliferation of fibroblasts and murine macrophage cell line RAW 264.7. The results of the assay for assessing the effect of paclitaxel on proliferation of murine RAW 264.7 macrophage cell line were shown in FIG. 18 (IC₅₀=134 nM).

Reference: In vitro toxicol. (1990) 3: 219; Biotech. Histochem. (1993) 68: 29; Anal. Biochem. (1993) 213: 426.

### Example 33

### Perivascular Administration of Paclitaxel to Assess Inhibition of Fibrosis

WISTAR rats weighing 250-300 g are anesthetized by the intramuscular injection of Innovar (0.33 ml/kg). Once sedated, they are then placed under Halothane anesthesia. After general anesthesia is established, fur over the neck region is shaved, the skin clamped and swabbed with betadine. A vertical incision is made over the left carotid artery and the external carotid artery exposed. Two ligatures are placed around the external carotid artery and a transverse arteriotomy is made. A number 2 French Fogarty balloon catheter is then introduced into the carotid artery and passed into the left common carotid artery and the balloon is inflated with saline. The catheter is passed up and down the carotid artery three times. The catheter is then removed and the ligature is tied off on the left external carotid artery.

Paclitaxel (33%) in ethelyne vinyl acetate (EVA) is then injected in a circumferential fashion around the common carotid artery in ten rats. EVA alone is injected around the common carotid artery in ten additional rats. (The paclitaxel may also be coated onto an EVA film which is then placed in a circumferential fashion around the common carotid artery.) Five rats from each group are sacrificed at 14 days and the final five at 28 days. The rats are observed for weight loss or other signs of systemic illness. After 14 or 28 days the animals are anesthetized and the left carotid artery is exposed in the manner of the initial experiment. The carotid artery is isolated, fixed at 10% buffered formaldehyde and examined for histology.

A statistically significant reduction in the degree of initimal hyperplasia, as measured by standard morphometric analysis, indicates a drug induced reduction in fibrotic response.

### Example 34

### MIC Determination by Microtitre Broth Dilution Method

A. MIC assay of various gram negative and positive bacteria MIC assays were conducted essentially as described by Amsterdam, D. 1996, “Susceptibility testing of antimicrobials in liquid media”, p.52-111, in Loman, V., ed. Antibiotics in laboratory medicine, 4th ed. Williams and Wilkins, Baltimore, Md. Briefly, a variety of compounds were tested for antibacterial activity against isolates of P. aeruginosa, K. pneumoniae, E. coli, S. epidermidus and S. aureus in the MIC (minimum inhibitory concentration assay under aerobic conditions using 96 well polystyrene microtitre plates (Falcon 1177), and Mueller Hinton broth at 37° C. incubated for 24 h. (MHB was used for most testing except C721 (S. pyogenes), which used Todd Hewitt broth, and Haemophilus influenzae, which used Haemophilus test medium (HTM)) Tests were conducted in triplicate. The results are provided below in Table 1.

TABLE 1
MINIMUM INHIBITORY CONCENTRATIONS OF THERAPEUTIC AGENTS
AGAINST VARIOUS GRAM NEGATIVE AND POSITIVE BACTERIA

| | Bacterial Strain | | | | | |
|---|---|---|---|---|---|---|
| | P. aeruginosa | K. pneumoniae | E. coli | S. aureus | | |
| | PAE/K799 | ATCC13883 | UB1005 | ATCC25923 | S. epidermidis | S. pyogenes |
| | H187 | C238 | C498 | C622 | C621 | C721 |
| | Wt | wt | wt | wt | wt | wt |
| Drug | Gram− | Gram− | Gram− | Gram+ | Gram+ | Gram+ |
| doxorubicin | 10⁻⁵ | 10⁻⁶ | 10⁻⁴ | 10⁻⁵ | 10⁻⁶ | 10⁻⁷ |
| mitoxantrone | 10⁻⁵ | 10⁻⁶ | 10⁻⁵ | 10⁻⁵ | 10⁻⁵ | 10⁻⁶ |
| 5-fluorouracil | 10⁻⁵ | 10⁻⁶ | 10⁻⁶ | 10⁻⁷ | 10⁻⁷ | 10⁻⁴ |
| methotrexate | N | 10⁻⁶ | N | 10⁻⁵ | N | 10⁻⁶ |
| etoposide | N | 10⁻⁵ | N | 10⁻⁵ | 10⁻⁶ | 10⁻⁵ |
| camptothecin | N | N | N | N | 10⁻⁴ | N |
| hydroxyurea | 10⁻⁴ | N | N | N | N | 10⁻⁴ |
| cisplatin | 10⁻⁴ | N | N | N | N | N |
| tubercidin | N | N | N | N | N | N |
| 2- | N | N | N | N | N | N |
| mercaptopurine | | | | | | |
| 6- | N | N | N | N | N | N |
| mercaptopurine | | | | | | |
| Cytarabine | N | N | N | N | N | N |

Activities are in Molar concentrations
Wt = wild type
N = No activity

B. MIC of Antibiotic-Resistant Bacteria

Various concentrations of the following compounds, mitoxantrone, cisplatin, tubercidin, methotrexate, 5-fluorouracil, etoposide, 2-mercaptopurine, doxorubicin, 6-mercaptopurine, camptothecin, hydroxyurea and cytarabine were tested for antibacterial activity against clinical isolates of a methicillin resistant S. aureus and a vancomycin resistant pediocoocus clinical isolate in an MIC assay as described above. Compounds which showed inhibition of growth (MIC value of <1.0×10−3) included: mitoxantrone (both strains), methotrexate (vancomycin resistant pediococcus), 5-fluorouracil (both strains), etoposide (both strains), and 2-mercaptopurine (vancomycin resistant pediococcus).

### Example 35

### Preparation of Release Buffer

The release buffer is prepared by adding 8.22 g sodium chloride, 0.32 g sodium phosphate monobasic (monohydrate) and 2.60 g sodium phosphate dibasic (anhydrous) to a beaker. 1 L HPLC grade water is added and the solution is stirred until all the salts are dissolved. If required, the pH of the solution is adjusted to pH 7.4±0.2 using either 0.1 N NaOH or 0.1 N phosphoric acid.

### Example 36

### Release Study to Determine Release Profile of a Therapeutic Agent from a Polymeric Composition

The release profile of a therapeutic agent from a polymeric composition can be determined according to the following procedure.

Release and Extraction

A sample is placed in a 16×125 mm screw capped culture tube. 16 ml release buffer (Example 35) is added to the tube. The samples are placed on a rotating wheel (30 rpm) in a 37° C. oven. At the various time intervals (2 h, 5 h, 8 h, 24 h and then daily), the sample tubes are taken from the oven, placed in a rack and the caps are removed in a fume hood. As much of the release buffer as possible is removed from the tube and placed in a second culture tube. 16 ml of release media is then added to the sample containing tube using an Oxford pipettor bottle. The samples are capped with a new PTFE lined cap. All samples are returned to the rotating wheel device in the oven.

Using a p1000 pipettor (PIPETMAN) and a clean pipette tip, remove and discard 1 ml of release media from each sample. Add 1 ml of dichloromethane to each sample using an oxford pipettor bottle. Cap each sample tube with the respective PTFE lined screw cap. Hand shake each sample vigorously for 5 seconds. Place samples on the labquake rotator and rotate for 15 min. Centrifuge samples at 1500 rpm for 10 minutes. Transfer the sample tubes to a fume hood and uncap. Remove most of the supernatant (aqueous phase) using a Pasteur pipette and vacuum system. Remove the final portion of the supernatant with a glass syringe. Transfer sample tubes to the pierce drying system, set the heating block to 1.5 (45° c.) and turn on the system. Dry all samples on the pierce drying system under a stream of nitrogen gas (approximately 45 min.). Re-cap the sample tubes, place in a plastic bag, label bag with date and time of sample, and store at −20 c. (freezer) until analysis.

External Standard Preparation

Paclitaxel (GMP grade) from Hauser Chemical Research, Inc. is be used as reference standard for this assay. Paclitaxel (100 mg) is be accurately weighed, quantitatively transferred and made up to volume with ACN in a 100 ml volumetric flask (1 mg/ml). Transfer 5 ml of this standard solution, using a volumetric pipette, to a 100 ml volumetric flask and make up to volume with ACN (50 μg/ml). Serial dilutions (5 ml qs ad 10 ml with ACN) will be used to prepare 25, 12.5, 6.25, 3.13, 1.56, 0.781 and 0.391 μg/ml solutions respectively. On the day of HPLC analysis of samples, place an aliquot (˜100 μl) of each standard into separate autosampler vials using small volume inserts and transfer to the HPLC.

Control and System Suitability Sample Preparation

Paclitaxel and 7-epi-taxel from Hauser Chemical Research, Inc. is used as control standards for this assay. Accurately weigh and quantitatively transfer 25 mg 7-Epi-taxel to a 25 ml volumetric flask and make up to volume with ACN (1 mg/ml). Transfer 5 ml of this standard solution, using a volumetric pipette, to a 100 ml flask and make up to volume with ACN (50 μg/ml 7-Epi-taxel). A 50/50 mixture of paclitaxel standard (25 μg/ml) and 7-epi-taxol standard (25 μg/ml) is used as the control and the system suitability samples. Prepare by adding a 5 ml aliquot of each paclitaxel dissolved in ACN (50 μg/ml paclitaxel) and 7-Epi-taxel dissolved in ACN (50 μg/ml 7-Epi-taxel) into the same culture tube. Cap and shake. Refrigerate until ready to use. On the day of HPLC analysis of samples, place an aliquot (˜150 μl) into two separate autosampler vials with small volume inserts and transfer to the HPLC. One sample is used for the system suitability. The other sample is used as the control sample.

Sample Reconstitution

Remove samples to be analyzed from the freezer, place in a fume hood, and allow tubes to come to room temperature. Uncap and add 1 ml of water/acetonitrile (50/50) to each tube with an Oxford pipettor. Recap sample tubes and vortex for 60 s. Centrifuge sample tubes at 1500 rpm for 15 min. In a fume hood, transfer approximately 500 μl of each sample to a separate HPLC autosampler vial with a clean Pasteur pipette. Cap each autosampler vial and transfer to the HPLC. Dispose of the sample tube and Pasteur pipette.

HPLC Analysis

The following chromatographic conditions are used for paclitaxel analysis:

| | |
|---|---|
| Stationary | ODS (Hypersil ODS, Hewlett Packard, |
| Phase | 125 × 4 mm ID, 5 μm) |
| Guard Column | Hypersil ODS Guard column |
| Mobile Phase | Acetonitrile(ACN)/Water(H₂O) 45/55 |
| Flow Rate | 1.0 ml/min |
| Injection | 10 μL |
| Volume | |
| Detection | Ultraviolet at 232 nm |
| Run Time | 15 min |
| Column | 28.0° C. |
| Temperature | |

Inject the acetonitrile sample five times at the beginning to ensure equilibration. Inject the control sample five times after the acetonitrile sample, once following the standard curve samples, once following every ten samples throughout the set of samples, and once at the end of the sample set to verify system performance. Chromatograph the standard curve samples by injecting once at the start of each set of samples.

Data Analysis

Integrate paclitaxel peak areas for all standards, control samples and release samples using HP ChemStation Batch Mode and generate a Batch Report saved in xls format. Use Excel to evaluate data from the Batch Report. Calculate the control sample peak area standard deviation (Excel: descriptive statistics) and % coefficient of variation (100× standard deviation/mean). Calculate the amount of paclitaxel injected (μg) for each standard curve sample based on the concentration prepared and a 10 μL injection. Calculate the slope and intercept of the standard curve (peak area versus amount of paclitaxel injected) using Excel: regression analysis. Calculate the amount of paclitaxel in each of the release samples injected. Establish the amount of paclitaxel (μg) released per 16 ml sample using the formula. The amount of paclitaxel released over time is established using the amount of paclitaxel per sample and the time the sample is taken.

### Example 37

### Formulation of a Drug in a Vehicle Comprising a Triblock Copolymer

Paclitaxel was incorporated into a formulation comprising a triblock copolymer and a diluent (described below) by dissolving the paclitaxel in the diluent with stirring at ambient temperature for at least two hours, then adding the triblock copolymer, again with stirring for at least 2 hours. Longer periods of time were used to add triblock copolymer at higher concentrations. For example, the addition of 33% triblock copolymer was accomplished by stirring for at least 15 hours (overnight). The diluent was PEG 300 NF or PEG 400 derivatized by end addition of trimethylene carbonate 90%/glycolide 10% in a ratio of 400:100. The triblock copolymer was an ABA copolymer with blocks A containing polymerized trimethylene carbonate (90%) and glycolide (10%), having a total molecular weight of about 900 g/mol and the B block containing PEG 400. Paclitaxel was effectively incorporated into this formulation at a concentration of 0.015 to 0.45 mg/ml. The amount of triblock copolymer in the formulation was varied from 2.3 to 50%w/w using PEG 400 as the diluent. The product was sterilized by exposure to about 2.5 kGy of gamma radiation.

### Example 38

### Formulation of a Drug in a Co-Solvent Vehicle

Paclitaxel was incorporated into a formulation comprising water and PEG 300 NF. The paclitaxel was first dissolved in a 90:10 mixture of PEG 300 NF:water by stirring at ambient temperature for at least two hours. Once the drug was dissolved, the composition was combined with equal parts of a 50:50 mixture of PEG 300 NF:water. The final composition was paclitaxel dissolved in a mixture of 70:30 PEG 300 NF:water. Paclitaxel was incorporated at concentrations of 0.45 to 4.5 mg/ml. The composition was passed through a 0.22 μm filter to render it sterile.

### Example 39

### Determination the Maximum Tolerated Dose (MTD) of a Drug After Intra-Articular Injection

Male Hartley guinea pigs, at least 6 weeks old, were anaesthetized using 5% isoflurane in an enclosed chamber. The animals were weighed and then transferred to the surgical table where anesthesia was maintained by nose cone with 2% isoflurane. The knee area on both legs was shaved and knee width at the head of the femur was measured on both knees. The skin on the right knee was sterilized. A 25G needle was introduced into the synovial cavity using a medial approach and 0.1 mL of the test formulation was injected. Three or seven days after the injection, the animals were sacrificed by cardiac injection of 0.7 mL Euthanyl under deep anesthesia (5% isoflurane). Sample size was N=3 for each formulation.

Knee function was assessed before sacrifice by recording changes in walking behavior and signs of tenderness. The animal was weighed immediately after sacrifice. The width of both knees at the head of the femur was then measured with calipers. The knee joint was dissected open by transecting the quadriceps tendon, cutting through the lateral and medial articular capsule and flipping the patella over the tibia. Knee inflammation was assessed by recording signs of swelling, vascularization, fluid accumulation and change in color in subcutaneous tissue as well as inner joint structures. Photographs were taken to document findings. All data was recorded by observers blinded to the treatment groups.

The MTD of the drug in the test formulation was determined to be that for which knee inflammation was not observed.

The MTD of paclitaxel in the Triblock Gel formulation from Example A was found to be 0.075 mg/ml, based upon evaluation at 7 days. Evaluation of this formulation after three days showed that doses up to 0.15 mg/ml were tolerated. The 0.015 mg/ml dose showed signs of inflammation only after seven days. The MTD of paclitaxel in the Co-Solvent formulation was found to be 1.5 mg/ml, based upon a 3 day evaluation.

### Example 40

### Evaluation of Local Tissue Distribution of a Drug After Intra-Articular Injection

Animals were injected in the knee joint as described above in 4.2 with the paclitaxel MTD dose identified for each formulation. Three or seven days after injection the animals were euthanized with an intracardiac injection of Euthanyl. The knee joint was dissected open and the synovial membrane, the anterior cruciate ligament, the fat pad, the menisci and the cartilage were harvested. Each tissue was briefly rinsed in saline solution, blotted dry and stored individually in a scintillation vial at −20° C. until paclitaxel analysis.

Paclitaxel was extracted from a weighed pooled sample from three animals by homogenization using a Polytron PT2000 homogenizer. The instrument setting was 3 to 9 and the extraction time was 1 minute. The extraction solution was 1 mL of 50/50 acetonitrile (ACN)/water containing 0.2 μg/mL 10-deacetyl taxol (10-DAT) and 0.1% formic acid. The extract was centrifuged using a Beckman J6-HC centrifuge for 10 minutes at 3000 rpm. The supernatant was filtered through an Acrodisc CR (13 mm, 0.45 μ) syringe filter into an HPLC vial for LC/MS/MS analysis. Some fat pad samples that did not produce a clear supernatant were centrifuged again prior to filtration using an IEC Micromax centrifuge for 10 minutes at 10000 rpm.

The paclitaxel content in the extract was determined by an LC/MS/MS method using an internal calibration. The calibration curve ranged from 0.01 to 1 μg/mL for Paclitaxel with 0.2 μg/mL 10-DAT. The LC/MS/MS system consists of a Waters 2695 separation module and a Waters Micromass QuattoMicro triple-Quad mass spectrometer. The LC method and the MS/MS method are described below.

LC method description

| | |
|---|---|
| Analytical Column | HPLC column: ACE 3 C18, |
| | 75 mm × 2.1 mm, 3 μm (particle size) |
| Guard Column | Upchurch C282 ODS |
| | 10 mm (length) × 2 mm |
| | (i.d.), 10 μm (particle size) |
| Mobile Phase | 60/40 ACN/Water (with 0.2% formic |
| | acid) |
| Flow Rate | 0.3 mL/min |
| Run Time | 5 min |
| Injection Volume | 10 μL |
| Column Temperature | 30° C. |
| Sample Temperature | 25° C. |

MS/MS method description

| | |
|---|---|
| Scan Type | MRM |
| | Channel 1: m/z 812.70 → 285.90 |
| | Channel 2: m/z 853.80 → 285.90 |
| Cone Voltage | 20.00 V |
| Collision Energy | 30.00 eV |
| Dwell | 0.50 s |
| Delay | 0.10 s |
| Run Time | 5 min |

Using this method it was demonstrated that measurable levels of paclitaxel were recovered from cartilage, menisci, ligament, fat and synovium of the treated animals. Drug tissue levels were maintained over at least a seven day period and additional studies have demonstrated that tissue levels may be maintained for periods of 21 to greater than 28 days, depending on the dose of paclitaxel administered. Furthermore paclitaxel delivered by injection of the formulation from Example A, with 0.015 mg/ml paclitaxel gave tissue concentrations that were six to eleven times greater than paclitaxel delivered at 15 mg/ml in PAXCEED, in ligament, fat, synovium and meniscal tissues. Thus it is an efficient delivery system for the drug.

### Example 41

### Evaluation of Local Tissue Distribution of a Drug after Intra Articular Injection

Animals are treated in the manner described in Example 39. Rabbits are evaluated by intra-articular injection of 0.5 ml of formulation. Paclitaxel is extracted from individual tissue sample from three animals by homogenization using a Freezer/Mill, SPEX CertiPrep 6850. The ground sample is extracted with 12 mL solution containing acetic acid (3.4 mM) and LiCl (4 to 8 μM) in 50/50 ACN/water. Extraction is performed on a Tube Rotator, Labquake Shaker for 30 minutes at room temperature. The extract is filtered through an Acrodisc CR (13 mm, 0.45 μ) syringe filter into an HPLC vial for LC/MS/MS analysis.

The paclitaxel content in the extract is determined by an LC/MS/MS method using an external calibration. The calibration curve ranges from 0.01 to 1 μg/mL for paclitaxel. The LC/MS/MS system consists of a Waters 2695 separation module and a Waters Micromass QUATTOMICRO triple-Quad mass spectrometer. The LC method and the MS/MS method are described below.

LC method description

| | |
|---|---|
| Analytical Column | HPLC column: ACE 3 C18, |
| | 75 mm × 2.1 mm, 3 μm (particle size) |
| Guard Column | Upchurch C282 ODS |
| | 10 mm (length) × 2 mm |
| | (i.d.), 10 μm (particle size) |
| Mobile Phase | 60/40 ACN/Water (with acetic acid, 3.4 mM |
| | and LiCl, 4 to 8 μM) |
| Flow Rate | 0.3 mL/min |
| Run Time | 5 min |
| Injection Volume | 10 μL |
| Column Temperature | 30° C. |
| Sample Temperature | 25° C. |

MS/MS method description

| | |
|---|---|
| Scan Type | MRM Channel: m/z 860 → 292 |
| Cone Voltage | 20.00 V |
| Collision Energy | 30.00 eV |
| Dwell | 0.50 s |
| Delay | 0.10 s |
| Run Time | 5 min |

Using this method it was demonstrated that paclitaxel was present in cartilage, menisci, ligament, fat and synovium of the treated animals at concentrations up to 3.25 μg/g tissue. Drug tissue levels were maintained over at a fourteen day period and that tissue levels may be maintained for periods of 21 to greater than 28 days, depending on the dose of paclitaxel administered.

### Example 42

### Spinal Surgical Adhesions Model to Assess Fibrosis Inhibiting Agents in Rabbits

Extensive scar formation and adhesions often occur after lumbar spine surgery involving the vertebrae. The dense and thick fibrous tissue adherent to the spine and adjacent muscles must be removed by surgery. Unfortunately, fibrous adhesions usually reform after the secondary surgery. Adhesions are formed by proliferation and migration of fibroblasts from the surrounding tissue at the site of surgery. These cells are responsible for the healing response after tissue injury. Once they have migrated to the wound they lay down proteins such as collagen to repair the injured tissue. Overproliferation and secretion by these cells induce local obstruction, compression and contraction of the surrounding tissues with accompanying side effects.

The rabbit laminectomy spinal adhesion model described herein is used to investigate spinal adhesion prevention by local slow release of antifibrotic drugs.

Five to six animals are included in each experimental group to allow for meaningful statistical analysis. Formulations with various concentrations of antifibrotic drugs are tested against control animals to assess inhibition of adhesion formation.

Rabbits are anesthetized with an IM injection of ketamine/zylazine. An endotracheal tube is inserted for maintenance of anesthesia with halothane. The animal is placed prone on the operating table on top of a heating pad and the skin over the lower half of the back is shaved and prepared for sterile surgery. A longitudinal midline skin incision is made from L-1 to L-5 and down the lumbosacral fascia. The fascia is incised to expose the tips of the spinous processes. The paraspinous muscles are dissected and retracted from the spinous process and lamina of L-4. A laminectomy is performed at L-4 by removal of the spinal process with careful bilateral excision of the laminae, thus creating a small 5×10 mm laminectomy defect. Hemostasis is obtained with Gelfoam. The test formulations are applied to the injury site and the wound is closed in layers with Vicryl sutures. The animals are placed in an incubator until recovery from anesthesia and then returned to their cage.

Two weeks after surgery, the animals are anesthetized using procedures similar to those described above. The animals are euthanized with Euthanyl. After a skin incision, the laminectomy site is analyzed by dissection and the amount of adhesion is scored using scoring systems published in the scientific literature for this type of injury.

### Example 43

### Tendon Surgical Adhesions Model to Assess Fibrosis Inhibiting Agents in Rabbits

This model is used to investigate whether adhesion of the tendons can be prevented by local slow release of drugs known to inhibit fibrosis. Polymeric formulations are loaded with drugs and implanted around injured tendons in rabbits. In animals without fibrosis-inhibiting formulations, adhesions develop within 3 weeks of flexor tendon injury if immobilization of the tendon is maintained during that period. An advantage of rabbits is that their tendon anatomy and cellular behaviour during tendon healing are similar to those in man except for the rate of healing that is much faster in rabbits.

Rabbits are anesthetized and the skin over the right hindlimb is shaved and prepared for sterile surgery. Sterile surgery is performed aided by an operating microscope. A longitudinal midline skin incision is made on the volvar aspect of the proximal phalange in digits 2 and 4. The synovial sheath of the tendons is carefully exposed and incised transversally to access the flexor digitorum profundus distal to the flexor digitorum superficialis bifurcation. Tendon injury is performed by gently lifting the flexor digitorum profundus with curved forceps and incising transversally through half of its substance. The formulation containing the test drug is applied around the tendons in the sheath of one of the two digits randomly selected. The other digit is left untreated and is used as a control. The sheath is then repaired with 6-0 nylon suture. An immobilizing 6-0 nylon suture is inserted through the transverse metacarpal ligament into the tendon/sheath complex to immobilize the tendon and the sheath as a single unit to encourage adhesion formation. The wound is closed with 4-0 interrupted sutures. A bandage is applied around the hindpaw to further augment immobilization of the digits and ensure comfort and ambulation of the animals. The animals are recovered and returned to their cage.

Three weeks after surgery, the animals are anesthetized. After a skin incision, the tissue plane around the synovial sheath is dissected and the tendon-sheath complex harvested en block and transferred in 10% phosphate buffered formaldehyde for histopathology analysis. The animals are then euthanized. After paraffin embedding, serial 5-um thin cross-sections are cut every 2 mm through the sheath and tendon complex. Sections are stained with H&E and Movat's stains to evaluate adhesion growth. Each slide is digitized using a computer connected to a digital microscope camera (Nikon Micropublisher cooled camera). Morphometry analysis is then performed using image analysis software (ImagePro). Thickness and area of adhesion defined as the substance obliterating the synovial space are measured and compared between formulation-treated and control animals.

### Example 44

### Assessment Paclitaxel in the Inhibition of Cartilage Damage in the ACL Injured Hartley Guinea Pig Model o Osteoarthritis

The purpose of this study was to determine whether paclitaxel administered in a hyaluronic acid formulation can delay or prevent the development of osteoarthritis in guinea pig knees. Surgical procedures.

Male Hartley guinea pigs, at least 6 weeks old, were anaesthetised using 5% isoflurane in an enclosed chamber. The animals were weighed and then transferred to the surgical table where anaesthesia was maintained by nose cone with 2% isoflurane. The knee area on the both legs was shaved and knee width at the head of the femur was measured on both knees. The skin on the right knee was sterilized. A 20G needle was introduced in the knee joint using a medial approach and the anterior cruciate ligament was cut with the sharp end of the needle. This procedure was practiced in a preliminary experiment that showed that the anterior cruciate ligament could be sectioned reliably using this technique.

Two weeks after the initial procedure, the animals were anesthetized with isoflurane (5% induction-2% maintenance) and weighed. The knee area on both legs was shaved and knee width at the head of the femur was measured on both knees. The skin of the injured knee was sterilised. A 25G needle was introduced into the synovial cavity using a medial approach and 0.1 ml of the test formulation was injected. Injections were repeated weekly for a total of 5 injections. Sample size was N=12 for each formulation. Two doses of paclitaxel and control formulation were tested.

Ten weeks after injury, the animals were sacrificed by cardiac injection of 0.7 ml Euthanyl under deep anaesthesia (5% isoflurane) and weighed. A final knee measurement was taken. The skin over the knee area was removed without damaging subcutaneous tissues. The knee joints were then harvested en bloc and placed into a formaldehyde (37%)/acetic acid solution (5:1 ratio) for fixation. Samples were sent to an independent laboratory for the conduct of histological preparation of joints and assessment by a pathologist for signs of cartilage damage.

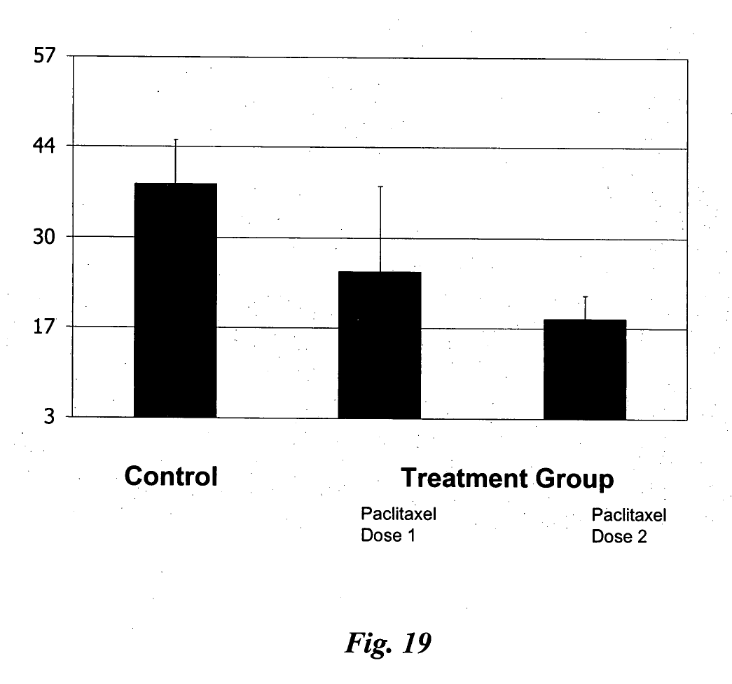

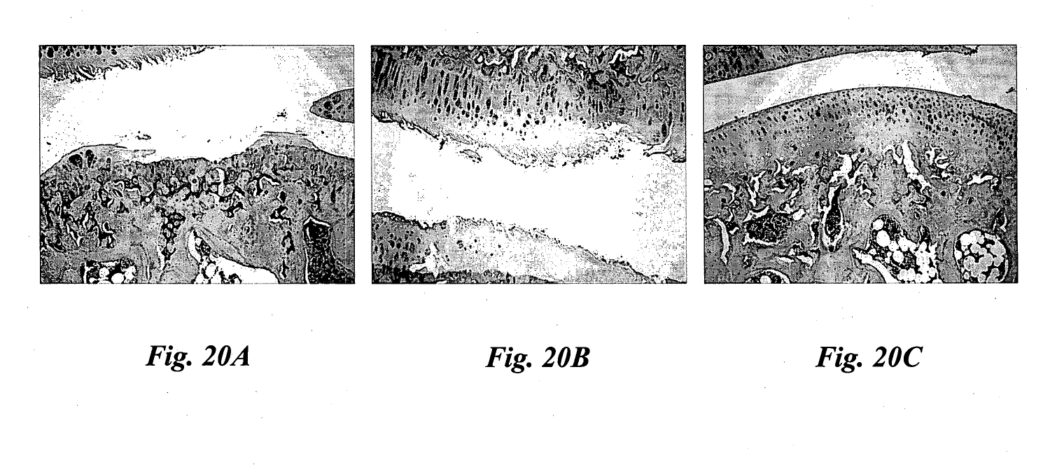

Briefly knee sections were made to examine cartilage and slides were stained with H&E stain. A pathologist scored slides in a blinded fashion from each animal using corresponding knee sections according to the following scale: no damage to cartilage, loss of proteoglycans, fraying of cartilage, loss of cartilage to the tidemark, and loss of cartilage to the bone. Bar graphs were constructed from each group and compared. Paclitaxel treatment at a low dose (dose 1) and medium dose (dose 2) showed a statistical reduction in cartilage damage relative to control. See FIGS. 19 and 20.

### Example 45

### Proteoglycan Loss Index in the Carrageenin-Induced and Antigen-Induced Rabbit Models of Arthritis Following Treatment with Paclitaxel

All microspheres were made using the oil in water solvent evaporation method described by Liggins and Burt (2001). The external phase was 100 ml of 1-5% PVA in water. The internal phase was 10 ml of a dichloromethane solution containing 5% w/v total solids (polymer and paclitaxel). The dispersion was stirred for 2 hours at room temperature to form microspheres. By varying the stirring speed between 900 and 2100 rpm and the PVA concentration, various size ranges were produced. The microspheres were separated from the external phase and rinsed with distilled water. Some microspheres were further divided into discrete size ranges by sieving the microspheres suspension through sieves having mesh sizes of 38, 53, 75 and 106 μm. Microsphere size distributions were determined using a Coulter LS130 particle size analyzer. Microspheres were suspended in water with a small amount of Tween 80 to prevent aggregation prior to particles size analysis. Chitosan microparticle size ranges were determined by optical microscopy using a microscope slide marked with 5 μm gradations. Optical microscopy was performed on both dry and wetted samples.

Thermal properties of the microspheres were determined using a Dupont Thermal Analysis DSC. Approximately 5 mg of microspheres were placed in unsealed aluminum pans and thermograms were obtained at a heating rate of 10° C./min. Evidence of crystallinity was obtained by X-ray powder diffraction measurements using a Rigaku X-ray diffractometer. Samples were scanned with a CuKa X-ray source through 5-35°2θ at a rate of 1°2θ/min with a step increment of 0.02°2θ.

The surface morphology of microspheres was determined using a Hitachi scanning electron microscope. Microspheres were coated with a 100 Å gold-palladium coat and visualized at a magnification of 1000 ×.

The paclitaxel content and in vitro release from microspheres were determined using the methods of Liggins & Burt (2001). For total content analysis, approximately 5 mg (accurately weighed) of microspheres were dissolved in 1 ml of dichloromethane followed by vigorous mixing with 15 ml of 60:40 acetonitrile:water. The solvent mixture was allowed to separate into two approximately equal volumes with a precipitated mass of polymer between the two. The amount of paclitaxel in each of the two fractions was then determined by HPLC using a Waters HPLC system. The mobile phase was 58:37:5 acetonitrile:water:methanol flowing at a rate of 1 ml/min. A 20 μl injection volume, a Novapak C18 column and UV detection at 232 nm were used.

Antigen induced arthritis was reproduced in rabbits using a previously described method (Kim et al., J. Rheumatol 1995:22:1714-21.). Briefly, female New Zealand white rabbits weighing 2.5-2.8 kg were used in biocompatibility and efficacy studies. Animals were housed in suspended caging with free access to food and water. Animals were acclimated for seven days prior to all experiments. Arthritis was induced in some animals for use as positive controls in biocompatibility testing and for use in efficacy studies. All knee joint injections were carried out under anaethesia induced by intramuscular injection of ketamine HCl (40 mg/kg) and xylazine (5 mg/kg). At the end of the in-life portion of the study, animals were sacrificed using intravenous T-61. The knee joints were dissected immediately after sacrifice and fixed in 10% formalin prior to histological analysis.

Antigen induced arthritis was established by three injections of bovine serum albumin (BSA) in Freund's complete adjuvant (FCA). The first injection consisted of 5 mg BSA emulsified in 1 ml FCA and diluted in 1 ml PBS. Three weeks later, each rabbit received a subcutaneous booster injection of 2.5 mg of BSA emulsified in 1 ml FCA diluted with 1 ml PBS. After four weeks, each rabbit received a second booster of 0.5 mg BSA in 0.3 mL pyrogen-free PBS injected into the knee joint. Five days after the final booster, the rabbits were treated by intra-articular injection with test articles.

Carrageenan induced arthritis was established in rabbits and the rabbits were treated in the same manner as for the antigen induced arthritis model. All rabbits in the carrageenan groups were injected with 0.3 ml of 1% carrageenan in pyrogen free PBS on days 1, 3, 8, 16 and 21. Half the animals were also injected with 35 mg of 20% paclitaxel-loaded microspheres on day 6. All animals were sacrificed on day 29 and the joints were dissected for histological analysis.

Synovial inflammation was assessed after sacrificing the rabbits. The joints were fixed in formalin and decalcified in 10% formic acid with repeated changes. The decalcified joints were embedded in paraffin and sections containing synovium, cartilage and bone were prepared. Sections were stained for cellularity with hematoxylin and eosin (H&E) and for proteoglycan content with safranin 0. Synovial inflammation and cartilage degradation were evaluated by blinded histological evaluation of parapatellar synovium and femoral condylar articular cartilage, respectively. Villus hyperplasia, fibroblast proliferation, fibrosis, angiogenesis, mononuclear cell and polymorphonuclear cell infiltrations were graded as indicators of synovial inflammation. For cartilage degradation, surface erosion, proteoglycan content and chondrocyte necrosis were graded. Grading of cellular infiltration and swelling was scored with an integer from 0 to 4 based on increasing erythema, swelling and cellular infiltration (0, normal; 4, maximum). For slight effects, a score of 0.5 was assigned; this was the only non-integer score used. Proteoglycan loss was also scored from 0 (normal) to 4 (almost total loss of stained proteoglycans).

The efficacy of paclitaxel-loaded polyester microspheres given by intra-articular injection in treating antigen induced arthritis was assessed using control and 20% loaded 10-35 and 35-105 μm PLA microspheres. Groups of five rabbits were treated with 40 mg of microspheres or PBS alone in the right joint. The left joint received PBS alone. The animals were sacrificed fourteen days after treatment and examined histologically for synovial inflammation and cartilage degradation as described above.

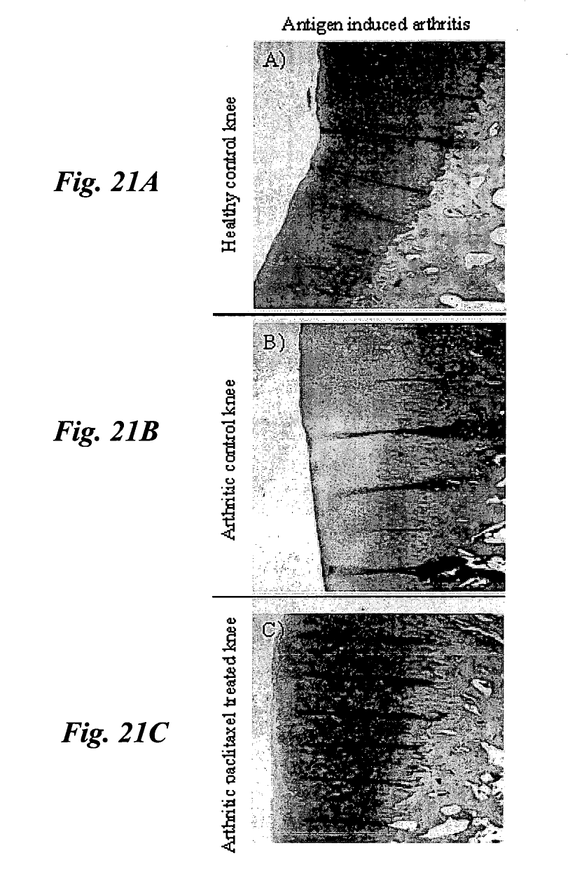

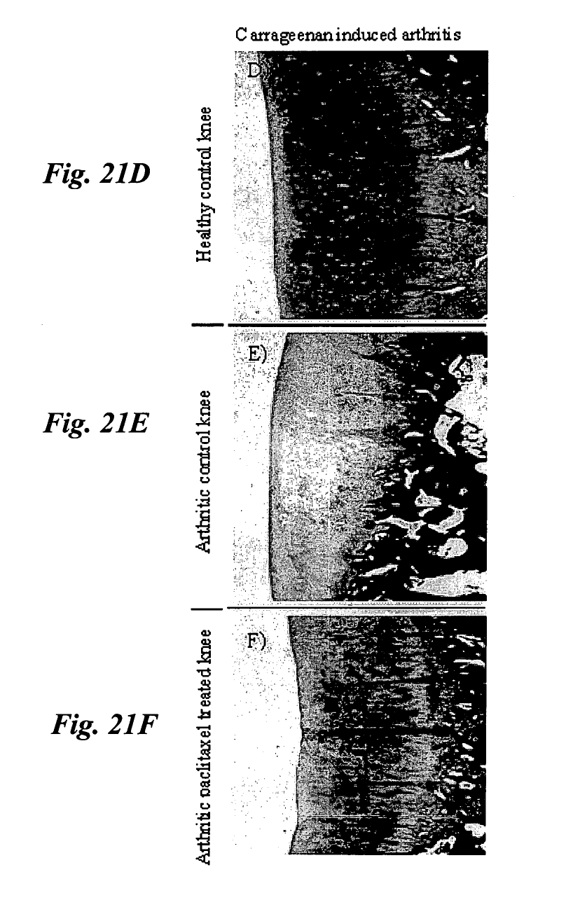

PLA microspheres containing 20% paclitaxel were selected for the efficacy study. Table 1 shows the results of the injection of 40 mg of control and paclitaxel-loaded PLA microspheres in rabbits with antigen induced arthritis. Untreated arthritic rabbits had a joint swelling score of 3 and 4.9×10⁷ cells in the joint fluid. Paclitaxel-loaded microspheres in the 10⁻³⁵ um size range did not reduce antigen induced arthritis. In fact, the amount of cellular infiltration was elevated in this group relative to untreated arthritic rabbits (Table 1). However, the injection of 35-105 μm paclitaxel-loaded microspheres significantly reduced both the joint swelling and the number of cells in the joint fluid (about a 50% decrease) relative to control (Table 1). Cartilage degradation expressed as proteoglycan loss and chondrocyte necrosis was also assessed in the control groups and the paclitaxel-loaded 35-105 pm microspheres group. There was no effect on either proteoglycan loss or chondrocyte necrosis by the injection of control PLA microspheres in diseased animals. However, animals treated with paclitaxel-loaded microspheres had significantly less proteoglycan loss than the untreated animals (Table 1 and FIGS. 21A-21C). FIG. 21A illustrates a knee having a normal histological appearance, with a continuous top layer of cartilage and no loss of stain color indicating normal proteoglycan content (score 0). FIG. 21 B shows a control microspheres arthritic knee with proteoglycan loss down to the bottom third layer of the section, which is termed heavy loss (score 3). In FIG. 21C, a paclitaxel microspheres treated arthritic knee shows only slight loss of proteoglycan at the surface layer of cartilage, with an intact surface (score 1).

The effect of paclitaxel-loaded microspheres in preventing proteoglycan loss in carrageenan induced arthritis was not as prominent as in antigen induced arthritis (FIGS. 21D-F). FIG. 21E shows severe loss of proteoglycan throughout all layers of cartilage, but the surface layer remained intact (score 4). Treatment of carrageenan induced knees with paclitaxel microspheres resulted in less reduction of stain color (FIG. 21 F, score 2), but the protective effect was not as pronounced as observed in the antigen induced model (FIG. 21C).

Antigen induced arthritis was used to determine efficacy in these studies. Although this animal model takes some time to develop, it mirrors many aspects of human rheumatoid arthritis such as the production of inflammatory cytokines (such as TNF-α), the loss of proteoglycans and the infiltration of white blood cells into the joint with chronic inflammation. Results from this model are compared to those from carrageenan-induced arthritis which is quick to establish in the rabbits and offers a method of inducing intense and reproducible levels of acute (rather than chronic) forms of arthritis. Because carrageenan-induced arthritis is characterized by severe proteoglycan loss, this model was also used in this study to measure the effect of intraarticular paclitaxel on proteoglycan loss. Efficacy studies that included measurements of joint swelling, cell infiltration, proteoglycan loss and chondrocyte necrosis demonstrated that the single injection of 40 mg of 20% paclitaxel-loaded, 35-105 μm microspheres significantly reduced all aspects of the chronic arthritic condition in rabbits (Table 1 and FIGS. 21A-C). The effect of paclitaxel-loaded microspheres in preventing proteoglycan loss in the carrageenan induced arthritis model was not as pronounced as for the antigen induced arthritis model.

TABLE 1
EFFICACY OF 40 MG OF CONTROL AND 20%
PACLITAXEL-LOADED PLA MICROSPHERES IN
THE SIZE RANGES OF 10-35 AND 35-105 MM,
ASSESSED IN TERMS OF MEAN SCORES (N = 5)
FOR SWELLING, CELLULAR INFILTRATION, LOSS OF
PROTEOGLYCAN AND CHONDROCYTE NECROSIS

| | Swelling | Number of | | Chondrocyte |
|---|---|---|---|---|
| | score | cell in joint | Proteoglycan | necrosis |
| Treatment | (0-4) | fluid | loss (0-4) | (0-3) |
| healthy, | 0 | 7.0 × 10⁵ | Not tested | Not tested |
| untreated | | | | |
| control | | | | |
| 35-105 μm, | 3 | 4.9 × 10⁷ | 2 ± 0.6 | 1 ± 0.3 |
| control | | | | |
| 10-35 μm, 20% | 3 | 8.4 × 10⁷ | Not tested | Not tested |
| paclitaxel | | | | |
| 35-105 μm, 20% | 1 | 2.4 × 10⁷ | 1 ± 0.3 | 0 ± 0.1 |
| paclitaxel | | | | |

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A method for implanting a medical device comprising: (a) infiltrating a tissue of a host where the medical device is to be, or has been, implanted with i) an anti-fibrotic agent, ii) an anti-infective agent, iii) a polymer; iv) a composition comprising an anti-fibrotic agent and a polymer, v) a composition comprising an anti-infective agent and a polymer, or vi) a composition comprising an anti-fibrotic agent, an anti-infective agent and a polymer, and (b) implanting the medical device into the host.

2. The method for implanting a medical device according to claim 1 comprising: (a) infiltrating a tissue of a host where the medical device is to be, or has been, implanted with an anti-fibrotic agent, and (b) implanting the medical device into the host.

3. The method for implanting a medical device according to claim 1 comprising: (a) infiltrating a tissue of a host where the medical device is to be, or has been, implanted with an anti-infective agent, and (b) implanting the medical device into the host.

4. The method for implanting a medical device according to claim 1 comprising: (a) infiltrating a tissue of a host where the medical device is to be, or has been, implanted with a polymer; and (b) implanting the medical device into the host.

5. The method for implanting a medical device according to claim 1 comprising: (a) infiltrating a tissue of a host where the medical device is to be, or has been, implanted with a composition comprising an anti-fibrotic agent and a polymer, and (b) implanting the medical device into the host.

6. The method for implanting a medical device according to claim 1 comprising: (a) infiltrating a tissue of a host where the medical device is to be, or has been, implanted with a composition comprising an anti-infective agent and a polymer, and (b) implanting the medical device into the host.

7. The method for implanting a medical device according to claim 1 comprising: (a) infiltrating a tissue of a host where the medical device is to be, or has been, implanted with a composition comprising an anti-fibrotic agent, an anti-infective agent and a polymer, and (b) implanting the medical device into the host.

8. The method according to claim 1 wherein the device is an intravascular device.

9. The method according to claim 1 wherein the device is a gastrointestinal stent.

10. The method according to claim 1 wherein the device is a tracheal and bronchial stent.

11. The method according to claim 1 wherein the device is a genital urinary stent.

12. The method according to claim 1 wherein the device is an ear and nose stent.

13. The method according to claim 1 wherein the device is an ear ventilation device.

14. The method according to claim 1 wherein the device is an intraocular implant.

15. The method according to claim 1 wherein the device is a vascular graft.

16. The method according to claim 1 wherein the device comprises a film or a mesh.

17. The method according to claim 1 wherein the device is a glaucoma drainage device.

18. The method according to claim 1 wherein the device is a prosthetic heart valve or a component thereof.

19. The method according to claim 1 wherein the device is a penile implant.

20. The method according to claim 1 wherein the device is an endotracheal or tracheostomy tube.

21. The method according to claim 1 wherein the device is a peritoneal dialysis catheter.

22. The method according to claim 1 wherein the device is a central nervous system shunt or a pressure monitoring device.

23. The method according to claim 1 wherein the device is an inferior vena cava filter.

24. The method according to claim 1 wherein the device is a gastrointestinal device.

25. The method according to claim 1 wherein the device is a central venous catheter.

26. The method according to claim 1 wherein the device is a ventricular assist device.

27. The method according to claim 1 wherein the device is a spinal implant.

28. The method according to claim 1 wherein the device is an implantable electrical device.

29. The method according to claim 1 wherein the device is an implantable sensor.

30. The method according to claim 1 wherein the device is an implantable pump.

31. The method according to claim 1 wherein the device is a soft tissue implant.

32. The method of claim 1 wherein the anti-fibrotic agent inhibits cell regeneration.

33. The method of claim 1 wherein the anti-fibrotic agent inhibits angiogenesis.

34. The method of claim 1 wherein the anti-fibrotic agent inhibits fibroblast migration.

35. The method of claim 1 wherein the anti-fibrotic agent inhibits fibroblast proliferation.

36. The method of claim 1 wherein the anti-fibrotic agent inhibits deposition of extracellular matrix.

37. The method of claim 1 wherein the anti-fibrotic agent inhibits tissue remodeling.

38.-39. (canceled)

40. The method of claim 1 wherein the anti-fibrotic agent is a chemokine receptor antagonist.

41. The method of claim 1 wherein the anti-fibrotic agent is a cell cycle inhibitor.

42. The method of claim 1 wherein the anti-fibrotic agent is a taxane.

43. The method of claim 1 wherein the anti-fibrotic agent is an anti-microtubule agent.

44. The method of claim 1 wherein the anti-fibrotic agent is paclitaxel.

45. The method of claim 1 wherein the anti-fibrotic agent is not paclitaxel.

46. The method of claim 1 wherein the anti-fibrotic agent is an analogue or derivative of paclitaxel.

47. The method of claim 1 wherein the anti-fibrotic agent is a vinca alkaloid.

48. The method of claim 1 wherein the anti-fibrotic agent is camptothecin or an analogue or derivative thereof.

49. The method of claim 1 wherein the anti-fibrotic agent is a podophyllotoxin.

50. The method of claim 1 wherein the anti-fibrotic agent is a podophyllotoxin, wherein the podophyllotoxin is etoposide or an analogue or derivative thereof.

51. The method of claim 1 wherein the anti-fibrotic agent is an anthracycline.

52. The method of claim 1 wherein the anti-fibrotic agent is an anthracycline, wherein the anthracycline is doxorubicin or an analogue or derivative thereof.

53. The method of claim 1 wherein the anti-fibrotic agent is an anthracycline, wherein the anthracycline is mitoxantrone or an analogue or derivative thereof.

54. The method of claim 1 wherein the anti-fibrotic agent is a platinum compound.

55. The method of claim 1 wherein the anti-fibrotic agent is a nitrosourea.

56. The method of claim 1 wherein the anti-fibrotic agent is a nitroimidazole.

57. The method of claim 1 wherein the anti-fibrotic agent is a folic acid antagonist.

58. The method of claim 1 wherein the anti-fibrotic agent is a cytidine analogue.

59. The method of claim 1 wherein the anti-fibrotic agent is a pyrimidine analogue.

60. The method of claim 1 wherein the anti-fibrotic agent is a fluoropyrimidine analogue.

61. The method of claim 1 wherein the anti-fibrotic agent is a purine analogue.

62. The method of claim 1 wherein the anti-fibrotic agent is a nitrogen mustard or an analogue or derivative thereof.

63. The method of claim 1 wherein the anti-fibrotic agent is a hydroxyurea.

64. The method of claim 1 wherein the anti-fibrotic agent is a mytomicin or an analogue or derivative thereof.

65-68. (canceled)

69. The method of claim 1 wherein the anti-fibrotic agent is a DNA alkylating agent.

70. The method of claim 1 wherein the anti-fibrotic agent is an anti-microtubule agent.

71. The method of claim 1 wherein the anti-fibrotic agent is a topoisomerase inhibitor.

72. The method of claim 1 wherein the anti-fibrotic agent is a DNA cleaving agent.

73. The method of claim 1 wherein the anti-fibrotic agent is an antimetabolite.

74. The method of claim 1 wherein the anti-fibrotic agent inhibits adenosine deaminase.

75. The method of claim 1 wherein the anti-fibrotic agent inhibits purine ring synthesis.

76. The method of claim 1 wherein the anti-fibrotic agent is a nucleotide interconversion inhibitor.

77. The method of claim 1 wherein the anti-fibrotic agent inhibits dihydrofolate reduction.

78. The method of claim 1 wherein the anti-fibrotic agent blocks thymidine monophosphate.

79. The method of claim 1 wherein the anti-fibrotic agent causes DNA damage.

80. The method of claim 1 wherein the anti-fibrotic agent is a DNA intercalation agent.

81. The method of claim 1 wherein the anti-fibrotic agent is a RNA synthesis inhibitor.

82. The method of claim 1 wherein the anti-fibrotic agent is a pyrimidine synthesis inhibitor.

83. The method of claim 1 wherein the anti-fibrotic agent inhibits ribonucleotide synthesis or finction.

84. The method of claim 1 wherein the anti-fibrotic agent inhibits thymidine monophosphate synthesis or function.

85. The method of claim 1 wherein the anti-fibrotic agent inhibits DNA synthesis.

86. The method of claim 1 wherein the anti-fibrotic agent causes DNA adduct formation.

87. The method of claim 1 wherein the anti-fibrotic agent inhibits protein synthesis.

88. The method of claim 1 wherein the anti-fibrotic agent inhibits microtubule function.

89. The method-of claim 1 wherein the anti-fibrotic agent is a cyclin dependent protein kinase inhibitor.

90.-189. (canceled)

190. The method of claim 1 wherein the anti-infective agent is-an anthracycline.

191. The method of claim 1 wherein the anti-infective agent is-doxorubicin.

192. The method of claim 1 wherein the anti-infective agent is ismitoxantrone.

193. The method of claim 1 wherein the anti-infective agent is a fluoropyrimidine.

194. The method of claim 1 wherein the anti-infective agent is 5-fluorouracil (5-FU).

195. The method of claim 1 wherein the anti-infective agent is a folic acid antagonist.

196. The method of claim 1 wherein the anti-infective agent is methotrexate.

197. The method of claim 1 wherein the anti-infective agent is a podophylotoxin.

198. The method of claim 1 wherein the anti-infective agent is etoposide.

199. The method of claim 1 wherein the anti-infective agent is camptothecin.

200. The method of claim 1 wherein the anti-infective agent is a hydroxyurea.

201. The method of claim 1 wherein the anti-infective agent is a platinum complex.

202. The method of claim 1 wherein the anti-infective agent is cisplatin.

203. The method of claim 1 wherein the composition comprises an anti-thrombotic agent.

204.-8822. (canceled)
